(19) United States
(12) Patent Application Publication
(10) Pub. No.: US 2024/0052042 A1
(43) Pub. Date: Feb. 15, 2024

(54) REVERSAL BINDING AGENTS FOR ANTI-NATRIURETIC PEPTIDE RECEPTOR I (NPRI) ANTIBODIES AND USES THEREOF

(71) Applicant: NOVARTIS AG, Basel (CH)

(72) Inventors: Yuichiro ADACHI, Arlington, MA (US); Jennifer R. ALLPORT-ANDERSON, Ipswich, MA (US); Shaun R. COUGHLIN, Cambridge, MA (US); John Louis DIENER, Cambridge, MA (US); Lars GADTKE, Munchen (DE); Felix HARTLEPP, Munchen (DE); Tiancen HU, Woodbridge, CT (US); Alexander Wolfgang KOCH, Arlington, MA (US); Kathrin LADETZKI-BAEHS, Planegg (DE); Michael John ROMANOWSKI, Cambridge, MA (US); Cesare RUSSO, Basel (CH); Xenia Karola WEZLER, Munchen (DE); Xiaoling XIE, Cambridge, MA (US)

(21) Appl. No.: 18/267,403

(22) PCT Filed: Dec. 13, 2021
(86) PCT No.: PCT/IB2021/061664

§ 371 Date: Jun. 14, 2023

Related U.S. Application Data
(60) Provisional application No. 63/125,031, filed on Dec. 14, 2020.

(51) Int. Cl. C07K 16/28; A61K 45/06; A61P 9/12

(52) U.S. Cl. CPC C07K 16/2863; A61K 45/06; A61P 9/12

(57) ABSTRACT

The present disclosure relates to use of reversal agents which specifically bind to anti-Natriuretic Peptide Receptor 1 (NPR1) antibodies or antigen binding fragments thereof, and reverse one or more effects of the anti-NPR1 antibody or antigen binding fragments thereof (e.g., hypotensive effects).

### BACKGROUND

Heart failure is a major public health problem concerning more than 20 million patients around the world (Orso et al., 2014, Expert Opin Pharmacother. 15(13):1849-1861) and is associated with high morbidity (Ibebuogu et al., 2011, Circulation. Heart failure 4(2):114-120). Natriuretic Peptide Receptor 1 (NPR1; also known as NPRA) is a receptor guanylate cyclase, which is activated by Atrial Natriuretic Peptide (ANP) resulting in lowering of blood pressure and blood volume (Chen & Burnett, 2006, European Heart Journal Supplements 8(Suppl E):E18-E25; Ibebuogu et al. 2011, supra; Mani et al. 2015, Bioscience Reports 35(5):e00260). ANP binding induces dimerization and twisting of the receptor that induces activation of the guanylate cyclase domain and conversion of GTP into cGMP (Misono et al., 2011, The FEBS journal 278(11):1818-1829). ANP is cleared by NPR3, a natriuretic peptide receptor that lacks the guanylate cyclase domain, and degraded by Neutral Endopeptidase (NEP) (Chen & Burnett 2006, supra; Schmitt et al., 2003, Clin Sci (Lond). 105(2):141-160). Certain antibodies against NPR1 have been described, for example, in WO2010/065293 and WO2020/086406.

It has been shown that an increase in ANP may be beneficial for patients with heart failure with reduced ejection fraction (outbound pumping of blood by heart). See McMurray et al., N. Engl. J. Med.; Vol. 371, No. 11, pp 993-1004 (2014); and Nougud et al., Eur J Heart Fail. 2019 May; 21(5):598-605. However, there is a need for further longer acting agents that have an alternative mode of action to supplement or replace existing therapies. Such therapies may have potential liabilities such as potential hypotension liabilities. See, e.g., Potter et al., Endocrine Reviews; Vol. 27, No. 1, pp 47-72 (2006); and Kamiya et al., Hear and Vessels; Vol. 35, No. 1; pp 59-68 (2019). Hypotension (e.g., severe hypotension) may result in an inadequate flow of blood to the body organs and may cause stroke, heart attack, kidney failure, shock, and death.

Specific reversal agents and methods of use of the same can be used as part of efforts to address the potential liabilities (e.g., the potential hypotension liability) for therapies incorporating long lasting agonists of NPR1 including, for example, in circumstances when reversal of the hypotensive effects of an anti-NPR1 agonist is needed for patient well-being.

### SUMMARY OF THE DISCLOSURE

Herein is demonstrated that it is possible to reverse the effects of activation or agonism of NPR1 (e.g., activation of NPR1 caused by the use of agonistic anti-NPR1 antibodies or antigen binding fragments thereof). This reversal may be effected through the use of a reversal agent (e.g., a binding agent that binds to the anti-NPR1 antibody or antigen binding fragment thereof. Such reversal may be used to treat, ameliorate, or prevent any disease or disorder caused by the agonism of NPR1 (e.g., through use of an anti-NPR1 antibody or antigen binding fragment). Such reversal may be used to treat, for example, the effects of the anti-NPR1 antibody or antigen binding fragment on cGMP levels in a subject who has been administered the anti-NPR1 antibody or antigen binding fragment. Such reversal may be used to treat, ameliorate, or prevent hypotension in a subject in need thereof (e.g., subject who has been administered the anti-NPR1 antibody or antigen binding fragment).

Thus the disclosure provides binding agents which specifically bind target anti-NPR1 antibodies (e.g., human monoclonal antibodies) or antigen-binding fragments thereof that (i) bind to natriuretic peptide receptor 1 (NPR1); and (ii) are capable of activating NPR1 in the absence of ANP. Such anti-NPR1 antibodies are agonistic anti-NPR1 antibodies. In some embodiments of the invention, the disclosure also provides binding agents which specifically bind target anti-NPR1 antibodies or antigen binding fragments thereof that (i) bind to natriuretic peptide receptor 1 (NPR1); and (ii) activate NPR1 in the absence of ANP. In some embodiments of the invention, the disclosure also provides binding agents which specifically bind target antibodies or antigen binding fragments thereof that (i) bind to natriuretic peptide receptor 1 (NPR1); and (ii) activate NPR1 in both the presence and absence of ANP.

Thus, in one aspect of the invention, herein is provided binding agents which specifically bind a target antibody or antigen binding fragment that (i) binds to natriuretic peptide receptor 1 (NPR1); and (ii) is capable of activating NPR1 in the absence of atrial natriuretic peptide (ANP). In some embodiments of the invention, the isolated antibody or antigen binding fragment does not bind to and/or does not activate natriuretic peptide receptor 2 (NPR2) and/or natriuretic peptide receptor 3 (NPR3). In some embodiments of the invention, the target antibody or antigen binding fragment binds to (a) human NPR1; and (b) mouse NPR1 and/or rat NPR1.

In some embodiments of the invention, the target (anti-NPR1) antibody or antigen binding fragment binds to (a) human NPR1; and (b) cyno NPR1. In some embodiments of the invention, the target (anti-NPR1) antibody or antigen binding fragment is ANP non-competitive. In some embodiments of the invention, the target (anti-NPR1) antibody or antigen binding fragment is ANP competitive. In some embodiments of the invention, the target (anti-NPR1) antibody or antigen binding fragment is capable of stabilizing the ANP-NPR1 complex.

In some embodiments of the invention, the target (anti-NPR1) antibody or antigen binding fragment binds to an epitope within amino acids 99-133 of SEQ ID NO: 1, e.g., within a region of human NPR1 encompassed by amino acids 99-133 of SEQ ID NO: 1. In some embodiments of the invention, the target (anti-NPR1) antibody or antigen binding fragment binds to an epitope comprising at least two amino acid residues within amino acids 99-133 of SEQ ID NO: 1. In some embodiments of the invention, the target (anti-NPR1) antibody or antigen binding fragment binds to an epitope comprising at least 3, 4, 5, 6, 7, or 8 amino acid residues within amino acids 99-133 of SEQ ID NO: 1.

In some embodiments of the invention, the target (anti-NPR1) antibody or antigen binding fragment binds to an epitope within amino acids 99-111 of SEQ ID NO: 1. In some embodiments of the invention, the target (anti-NPR1) antibody or antigen binding fragment binds to an epitope within amino acids 99-103 of SEQ ID NO: 1. In some embodiments of the invention, the target (anti-NPR1) antibody or antigen binding fragment binds to an epitope within amino acids 105-111 of SEQ ID NO: 1. In some embodiments of the invention, the target (anti-NPR1) antibody or antigen binding fragment binds to an epitope comprising at least 2, 3, or 4 amino acid residues within amino acids 105-111 of SEQ ID NO: 1. In some embodiments of the invention, the target (anti-NPR1) antibody or antigen binding fragment binds to a conformational epitope of human NPR1, and wherein the conformational epitope comprises at least one amino acid residue within each of (i) amino acids 99-103 of SEQ ID NO: 1, (ii) 105-111 of SEQ ID NO: 1, (iii) 131-134 of SEQ ID NO: 1, and additionally binds to amino acid 375 and/or 378 of SEQ ID NO: 1. In some embodiments of the invention, the epitope is a conformational epitope, and the conformational epitope additionally comprises at least one amino acid residue selected from the group consisting of amino acids 33, 34, 76, 82, and 104 of SEQ ID NO: 1. In some embodiments of the invention, the conformational epitope additionally comprises at least one amino acid residue selected from the group consisting of amino acids 33, 34, 76, 82, 104, 374, and 375 of SEQ ID NO: 1.

In some embodiments of the invention, the target (anti-NPR1) antibody or antigen binding fragment binds to at least amino acids 82, 102, 103, 105, 106, 109, 132, and 375 of SEQ ID NO: 1. In some embodiments of the invention, the target (anti-NPR1) antibody or antigen binding fragment binds to at least amino acids 34, 82, 102, 103, 105, 106, 107, 109, 132, 133, 375, and 378 of SEQ ID NO: 1. In some embodiments of the invention, the target (anti-NPR1) antibody or antigen binding fragment binds to at least amino acids 79, 82, 99, 102, 103, 105, 106, 109, 131, 132, and 375 of SEQ ID NO: 1.

In some embodiments of the invention, the target (anti-NPR1) antibody or antigen binding fragment binds to an epitope within amino acids 188-219 of SEQ ID NO: 1. In some embodiments of the invention, the target (anti-NPR1) antibody or antigen binding fragment binds to an epitope comprising at least 2, 3, 4, 5, 6, or 7 amino acids within amino acids 188-219 of SEQ ID NO: 1. In some embodiments of the invention, the target (anti-NPR1) antibody or antigen binding fragment binds to a conformational epitope within NPR1, and the conformational epitope comprises at least one amino acid residue within each of (i) amino acids 188-198 of SEQ ID NO: 1, (ii) 201-208 of SEQ ID NO: 1, (iii) 215-238 of SEQ ID NO: 1, and (iv) 294-297 of SEQ ID NO: 1. In some embodiments of the invention, the target (anti-NPR1) antibody or antigen binding fragment binds to at least amino acids 188, 192, 194, 197, 201, 208, and 219 of SEQ ID NO: 1. In some embodiments of the invention, the target (anti-NPR1) antibody or antigen binding fragment binds to at least amino acids 188, 192, 194, 197, 201, 208, 219, and 295 of SEQ ID NO: 1.

In some embodiments of the invention, the target (anti-NPR1) antibody or antigen binding fragment comprises three heavy chain complementarity determining regions (HCDR1, HCDR2, and HCDR3) and three light chain complementarity determining regions (LCDR1, LCDR2, and LCDR3) selected from: (a) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 135 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 134 (LCDR3); (b) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 126 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 126 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 127 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 126 (LCDR3); (c) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 145 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 145 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 146 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 145 (LCDR3); (d) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 172 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 172 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 173 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 172 (LCDR3); (e) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 178 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 178 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 179 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 178 (LCDR3); (f) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 184 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 184 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 185 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 184 (LCDR3); (g) (I) SEQ ID NO: 4 (HCDR1), SEQ ID NO: 100 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1). SEQ ID NO: 100 (HCDR2). SEQ ID NO: 6 (HCDR3). SEQ ID NO: 17 (LCDR1), SEQ ID NO: 18 (LCDR2), and SEQ ID NO: 19 (LCDR3); (III) SEQ ID NO: 8 (HCDR1), SEQ ID NO: 101 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 10 (HCDR1), SEQ ID NO: 102 (HCDR2), SEQ ID NO: 12 (HCDR3), SEQ ID NO: 23 (LCDR1), SEQ ID NO: 21 (LCDR2), and SEQ ID NO: 19 (LCDR3); (h) (I) SEQ ID NO: 112 (HCDR1), SEQ ID NO: 100 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 100 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 113 (HCDR1), SEQ ID NO: 101 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 114 (HCDR1), SEQ ID NO: 102 (HCDR2), SEQ ID NO: 12 (HCDR3), SEQ ID NO: 23 (LCDR1), SEQ ID NO: 21 (LCDR2), and SEQ ID NO: 19 (LCDR3); (i) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 43 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 43 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 46 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 43 (LCDR3); (j) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 126 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 126 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 127 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 126 (LCDR3); (k) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 134 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 134 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 135 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 134 (LCDR3); (1) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 145 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 145 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 146 (LCDR3); or (IV) SEQ ID NO: 34 (HCDR1), SEQ ID NO: 35 (HCDR2). SEQ ID NO: 36 (HCDR3). SEQ ID NO: 47 (LCDR1). SEQ ID NO: 45 (LCDR2), and SEQ ID NO: 145 (LCDR3); (m) (I) SEQ ID NO: 4 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 8 (HCDR1), SEQ ID NO: 152 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 10 (HCDR1), SEQ ID NO: 153 (HCDR2), SEQ ID NO: 12 (HCDR3), SEQ ID NO: 23 (LCDR1), SEQ ID NO: 21 (LCDR2), and SEQ ID NO: 19 (LCDR3); (n) (I) SEQ ID NO: 112 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 113 (HCDR1), SEQ ID NO: 152 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 114 (HCDR1), SEQ ID NO: 153 (HCDR2), SEQ ID NO: 12 (HCDR3), SEQ ID NO: 23 (LCDR1), SEQ ID NO: 21 (LCDR2), and SEQ ID NO: 19 (LCDR3); (o) (I) SEQ ID NO: 165 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 166 (HCDR1), SEQ ID NO: 152 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 167 (HCDR1), SEQ ID NO: 153 (HCDR2), SEQ ID NO: 12 (HCDR3), SEQ ID NO: 23 (LCDR1), SEQ ID NO: 21 (LCDR2), and SEQ ID NO: 19 (LCDR3); (p) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 172 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 172 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 173 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 172 (LCDR3); (q) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 178 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 178 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 179 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 178 (LCDR3); (r) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 184 (LCDR3); (II) SEQ ID NO: 31 (HCDR1), SEQ ID NO: 29 (HCDR2), SEQ ID NO: 30 (HCDR3), SEQ ID NO: 41 (LCDR1). SEQ ID NO: 42 (LCDR2), and SEQ ID NO: 184 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 185 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 184 (LCDR3); (s) (I) SEQ ID NO: 28 (HCDR1), SEQ ID NO: 190 (HCDR2), SEQ ID NO: 30 (HCDR3), SEQ ID NO: 41 (LCDR1), SEQ ID NO: 42 (LCDR2), and SEQ ID NO: 134 (LCDR3); (II) SEQ ID NO: 31 (HCDR1), SEQ ID NO: 190 (HCDR2), SEQ ID NO: 30 (HCDR3), SEQ ID NO: 41 (LCDR1), SEQ ID NO: 42 (LCDR2), and SEQ ID NO: 134 (LCDR3); (III) SEQ ID NO: 32 (HCDR1), SEQ ID NO: 191 (HCDR2), SEQ ID NO: 30 (HCDR3), SEQ ID NO: 44 (LCDR1), SEQ ID NO: 45 (LCDR2), and SEQ ID NO: 135 (LCDR3); or (IV) SEQ ID NO: 34 (HCDR1), SEQ ID NO: 192 (HCDR2), SEQ ID NO: 36 (HCDR3), SEQ ID NO: 47 (LCDR1), SEQ ID NO: 45 (LCDR2), and SEQ ID NO: 134 (LCDR3); (t) (I) SEQ ID NO: 28 (HCDR1), SEQ ID NO: 190 (HCDR2), SEQ ID NO: 30 (HCDR3), SEQ ID NO: 41 (LCDR1), SEQ ID NO: 42 (LCDR2), and SEQ ID NO: 172 (LCDR3); (II) SEQ ID NO: 31 (HCDR1), SEQ ID NO: 190 (HCDR2), SEQ ID NO: 30 (HCDR3), SEQ ID NO: 41 (LCDR1), SEQ ID NO: 42 (LCDR2), and SEQ ID NO: 172 (LCDR3); (III) SEQ ID NO: 32 (HCDR1), SEQ ID NO: 191 (HCDR2), SEQ ID NO: 30 (HCDR3), SEQ ID NO: 44 (LCDR1), SEQ ID NO: 45 (LCDR2), and SEQ ID NO: 173 (LCDR3); or (IV) SEQ ID NO: 34 (HCDR1), SEQ ID NO: 192 (HCDR2), SEQ ID NO: 36 (HCDR3), SEQ ID NO: 47 (LCDR1), SEQ ID NO: 45 (LCDR2), and SEQ ID NO: 172 (LCDR3); (u) (I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 19 (LCDR3); (v) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 43 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 43 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 46 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 43 (LCDR3); (w) (I) SEQ ID NO: 367 (HCDR1), SEQ ID NO: 368 (HCDR2), SEQ ID NO: 228 (HCDR3), SEQ ID NO: 237 (LCDR1), SEQ ID NO: 238 (LCDR2), and SEQ ID NO: 239 (LCDR3); (II) SEQ ID NO: 369 (HCDR1), SEQ ID NO: 368 (HCDR2), SEQ ID NO: 228 (HCDR3), SEQ ID NO: 237 (LCDR1), SEQ ID NO: 238 (LCDR2), and SEQ ID NO: 239 (LCDR3); (III) SEQ ID NO: 370 (HCDR1), SEQ ID NO: 371 (HCDR2), SEQ ID NO: 228 (HCDR3), SEQ ID NO: 240 (LCDR1), SEQ ID NO: 241 (LCDR2), and SEQ ID NO: 242 (LCDR3); or (IV) SEQ ID NO: 372 (HCDR1), SEQ ID NO: 373 (HCDR2), SEQ ID NO: 232 (HCDR3), SEQ ID NO: 243 (LCDR1), SEQ ID NO: 241 (LCDR2), and SEQ ID NO: 239 (LCDR3); (x) (I) SEQ ID NO: 378 (HCDR1), SEQ ID NO: 379 (HCDR2), SEQ ID NO: 228 (HCDR3), SEQ ID NO: 237 (LCDR1), SEQ ID NO: 238 (LCDR2), and SEQ ID NO: 239 (LCDR3); (II) SEQ ID NO: 380 (HCDR1), SEQ ID NO: 379 (HCDR2), SEQ ID NO: 228 (HCDR3), SEQ ID NO: 237 (LCDR1), SEQ ID NO: 238 (LCDR2), and SEQ ID NO: 239 (LCDR3); (III) SEQ ID NO: 381 (HCDR1), SEQ ID NO: 382 (HCDR2), SEQ ID NO: 228 (HCDR3), SEQ ID NO: 240 (LCDR1), SEQ ID NO: 241 (LCDR2), and SEQ ID NO: 242 (LCDR3); or (IV) SEQ ID NO: 383 (HCDR1), SEQ ID NO: 384 (HCDR2), SEQ ID NO: 232 (HCDR3), SEQ ID NO: 243 (LCDR1), SEQ ID NO: 241 (LCDR2), and SEQ ID NO: 239 (LCDR3); (y) (I) SEQ ID NO: 226 (HCDR1), SEQ ID NO: 227 (HCDR2), SEQ ID NO: 228 (HCDR3), SEQ ID NO: 237 (LCDR1), SEQ ID NO: 238 (LCDR2), and SEQ ID NO: 239 (LCDR3); (II) SEQ ID NO: 229 (HCDR1), SEQ ID NO: 227 (HCDR2), SEQ ID NO: 228 (HCDR3), SEQ ID NO: 237 (LCDR1), SEQ ID NO: 238 (LCDR2), and SEQ ID NO: 239 (LCDR3); (III) SEQ ID NO: 32 (HCDR1), SEQ ID NO: 230 (HCDR2), SEQ ID NO: 228 (HCDR3), SEQ ID NO: 240 (LCDR1), SEQ ID NO: 241 (LCDR2), and SEQ ID NO: 242 (LCDR3); or (IV) SEQ ID NO: 34 (HCDR1), SEQ ID NO: 231 (HCDR2), SEQ ID NO: 232 (HCDR3), SEQ ID NO: 243 (LCDR1), SEQ ID NO: 241 (LCDR2), and SEQ ID NO: 239 (LCDR3); (z) (I) SEQ ID NO: 270 (HCDR1), SEQ ID NO: 271 (HCDR2), SEQ ID NO: 272 (HCDR3), SEQ ID NO: 282 (LCDR1), SEQ ID NO: 261 (LCDR2), and SEQ ID NO: 283 (LCDR3); (II) SEQ ID NO: 273 (HCDR1), SEQ ID NO: 271 (HCDR2), SEQ ID NO: 272 (HCDR3), SEQ ID NO: 282 (LCDR1), SEQ ID NO: 261 (LCDR2), and SEQ ID NO: 283 (LCDR3); (III) SEQ ID NO: 32 (HCDR1), SEQ ID NO: 274 (HCDR2), SEQ ID NO: 272 (HCDR3), SEQ ID NO: 284 (LCDR1), SEQ ID NO: 241 (LCDR2), and SEQ ID NO: 285 (LCDR3); or (IV) SEQ ID NO: 275 (HCDR1), SEQ ID NO: 276 (HCDR2), SEQ ID NO: 277 (HCDR3), SEQ ID NO: 286 (LCDR1), SEQ ID NO: 241 (LCDR2), and SEQ ID NO: 283 (LCDR3); or (aa) (I) SEQ ID NO: 291 (HCDR1), SEQ ID NO: 292 (HCDR2), SEQ ID NO: 293 (HCDR3), SEQ ID NO: 237 (LCDR1), SEQ ID NO: 238 (LCDR2), and SEQ ID NO: 304 (LCDR3); (II) SEQ ID NO: 294 (HCDR1), SEQ ID NO: 292 (HCDR2), SEQ ID NO: 293 (HCDR3), SEQ ID NO: 237 (LCDR1), SEQ ID NO: 238 (LCDR2), and SEQ ID NO: 304 (LCDR3); (III) SEQ ID NO: 295 (HCDR1), SEQ ID NO: 296 (HCDR2), SEQ ID NO: 293 (HCDR3), SEQ ID NO: 240 (LCDR1), SEQ ID NO: 241 (LCDR2), and SEQ ID NO: 305 (LCDR3); or (IV) SEQ ID NO: 297 (HCDR1), SEQ ID NO: 298 (HCDR2), SEQ ID NO: 299 (HCDR3), SEQ ID NO: 243 (LCDR1), SEQ ID NO: 241 (LCDR2), and SEQ ID NO: 304 (LCDR3).

In some embodiments of the invention, the target (anti-NPR1) antibody or antigen binding fragment comprises three heavy chain complementarity determining regions (HCDR1, HCDR2, and HCDR3) and three light chain complementarity determining regions (LCDR1, LCDR2, and LCDR3) selected from: (a) (I) SEQ ID NO: 52 (HCDR1), SEQ ID NO: 53 (HCDR2), SEQ ID NO: 54 (HCDR3), SEQ ID NO: 65 (LCDR1), SEQ ID NO: 66 (LCDR2), and SEQ ID NO: 67 (LCDR3); (II) SEQ ID NO: 55 (HCDR1), SEQ ID NO: 53 (HCDR2), SEQ ID NO: 54 (HCDR3), SEQ ID NO: 65 (LCDR1), SEQ ID NO: 66 (LCDR2), and SEQ ID NO: 67 (LCDR3); (III) SEQ ID NO: 56 (HCDR1), SEQ ID NO: 57 (HCDR2), SEQ ID NO: 54 (HCDR3), SEQ ID NO: 68 (LCDR1), SEQ ID NO: 69 (LCDR2), and SEQ ID NO: 70 (LCDR3); or (IV) SEQ ID NO: 58 (HCDR1), SEQ ID NO: 59 (HCDR2), SEQ ID NO: 60 (HCDR3). SEQ ID NO: 71 (LCDR1). SEQ ID NO: 69 (LCDR2), and SEQ ID NO: 67 (LCDR3); (b) (I) SEQ ID NO: 76 (HCDR1), SEQ ID NO: 77 (HCDR2), SEQ ID NO: 78 (HCDR3), SEQ ID NO: 89 (LCDR1), SEQ ID NO: 90 (LCDR2), and SEQ ID NO: 91 (LCDR3); (II) SEQ ID NO: 79 (HCDR1), SEQ ID NO: 77 (HCDR2), SEQ ID NO: 78 (HCDR3), SEQ ID NO: 89 (LCDR1), SEQ ID NO: 90 (LCDR2), and SEQ ID NO: 91 (LCDR3); (III) SEQ ID NO: 80 (HCDR1), SEQ ID NO: 81 (HCDR2), SEQ ID NO: 78 (HCDR3), SEQ ID NO: 92 (LCDR1), SEQ ID NO: 93 (LCDR2), and SEQ ID NO: 94 (LCDR3); or (IV) SEQ ID NO: 82 (HCDR1), SEQ ID NO: 83 (HCDR2), SEQ ID NO: 84 (HCDR3), SEQ ID NO: 95 (LCDR1), SEQ ID NO: 93 (LCDR2), and SEQ ID NO: 91 (LCDR3); (c) (I) SEQ ID NO: 310 (HCDR1), SEQ ID NO: 311 (HCDR2), SEQ ID NO: 348 (HCDR3), SEQ ID NO: 320 (LCDR1), SEQ ID NO: 354 (LCDR2), and SEQ ID NO: 361 (LCDR3); (II) SEQ ID NO: 229 (HCDR1), SEQ ID NO: 311 (HCDR2), SEQ ID NO: 348 (HCDR3), SEQ ID NO: 320 (LCDR1), SEQ ID NO: 354 (LCDR2), and SEQ ID NO: 361 (LCDR3); (III) SEQ ID NO: 80 (HCDR1), SEQ ID NO: 313 (HCDR2), SEQ ID NO: 348 (HCDR3), SEQ ID NO: 323 (LCDR1), SEQ ID NO: 324 (LCDR2), and SEQ ID NO: 362 (LCDR3); or (IV) SEQ ID NO: 82 (HCDR1), SEQ ID NO: 314 (HCDR2), SEQ ID NO: 349 (HCDR3), SEQ ID NO: 326 (LCDR1), SEQ ID NO: 324 (LCDR2), and SEQ ID NO: 361 (LCDR3); (d) (I) SEQ ID NO: 270 (HCDR1), SEQ ID NO: 389 (HCDR2), SEQ ID NO: 331 (HCDR3), SEQ ID NO: 337 (LCDR1), SEQ ID NO: 338 (LCDR2), and SEQ ID NO: 339 (LCDR3); (II) SEQ ID NO: 273 (HCDR1), SEQ ID NO: 389 (HCDR2), SEQ ID NO: 331 (HCDR3), SEQ ID NO: 337 (LCDR1), SEQ ID NO: 338 (LCDR2), and SEQ ID NO: 339 (LCDR3); (III) SEQ ID NO: 32 (HCDR1), SEQ ID NO: 390 (HCDR2), SEQ ID NO: 331 (HCDR3), SEQ ID NO: 340 (LCDR1), SEQ ID NO: 341 (LCDR2), and SEQ ID NO: 342 (LCDR3); or (IV) SEQ ID NO: 275 (HCDR1), SEQ ID NO: 391 (HCDR2), SEQ ID NO: 332 (HCDR3), SEQ ID NO: 343 (LCDR1), SEQ ID NO: 341 (LCDR2), and SEQ ID NO: 339 (LCDR3); (e) (I) SEQ ID NO: 407 (HCDR1), SEQ ID NO: 408 (HCDR2), SEQ ID NO: 331 (HCDR3), SEQ ID NO: 337 (LCDR1), SEQ ID NO: 338 (LCDR2), and SEQ ID NO: 339 (LCDR3); (II) SEQ ID NO: 409 (HCDR1), SEQ ID NO: 408 (HCDR2), SEQ ID NO: 331 (HCDR3), SEQ ID NO: 337 (LCDR1), SEQ ID NO: 338 (LCDR2), and SEQ ID NO: 339 (LCDR3); (III) SEQ ID NO: 410 (HCDR1), SEQ ID NO: 411 (HCDR2), SEQ ID NO: 331 (HCDR3), SEQ ID NO: 340 (LCDR1), SEQ ID NO: 341 (LCDR2), and SEQ ID NO: 342 (LCDR3); or (IV) SEQ ID NO: 412 (HCDR1), SEQ ID NO: 413 (HCDR2), SEQ ID NO: 332 (HCDR3), SEQ ID NO: 343 (LCDR1), SEQ ID NO: 341 (LCDR2), and SEQ ID NO: 339 (LCDR3); (f) (I) SEQ ID NO: 310 (HCDR1), SEQ ID NO: 311 (HCDR2), SEQ ID NO: 312 (HCDR3), SEQ ID NO: 320 (LCDR1), SEQ ID NO: 321 (LCDR2), and SEQ ID NO: 322 (LCDR3); (II) SEQ ID NO: 229 (HCDR1), SEQ ID NO: 311 (HCDR2), SEQ ID NO: 312 (HCDR3), SEQ ID NO: 320 (LCDR1), SEQ ID NO: 321 (LCDR2), and SEQ ID NO: 322 (LCDR3); (III) SEQ ID NO: 80 (HCDR1), SEQ ID NO: 313 (HCDR2), SEQ ID NO: 312 (HCDR3), SEQ ID NO: 323 (LCDR1), SEQ ID NO: 324 (LCDR2), and SEQ ID NO: 325 (LCDR3); or (IV) SEQ ID NO: 82 (HCDR1), SEQ ID NO: 314 (HCDR2), SEQ ID NO: 315 (HCDR3), SEQ ID NO: 326 (LCDR1), SEQ ID NO: 324 (LCDR2), and SEQ ID NO: 322 (LCDR3); (g) (I) SEQ ID NO: 270 (HCDR1), SEQ ID NO: 271 (HCDR2), SEQ ID NO: 331 (HCDR3), SEQ ID NO: 337 (LCDR1), SEQ ID NO: 338 (LCDR2), and SEQ ID NO: 339 (LCDR3); (II) SEQ ID NO: 273 (HCDR1), SEQ ID NO: 271 (HCDR2), SEQ ID NO: 331 (HCDR3), SEQ ID NO: 337 (LCDR1), SEQ ID NO: 338 (LCDR2), and SEQ ID NO: 339 (LCDR3); (III) SEQ ID NO: 32 (HCDR1), SEQ ID NO: 274 (HCDR2), SEQ ID NO: 331 (HCDR3), SEQ ID NO: 340 (LCDR1), SEQ ID NO: 341 (LCDR2), and SEQ ID NO: 342 (LCDR3); or (IV) SEQ ID NO: 275 (HCDR1), SEQ ID NO: 276 (HCDR2), SEQ ID NO: 332 (HCDR3), SEQ ID NO: 343 (LCDR1), SEQ ID NO: 341 (LCDR2), and SEQ ID NO: 339 (LCDR3); or (h) (I) SEQ ID NO: 310 (HCDR1), SEQ ID NO: 311 (HCDR2), SEQ ID NO: 348 (HCDR3), SEQ ID NO: 320 (LCDR1), SEQ ID NO: 354 (LCDR2), and SEQ ID NO: 355 (LCDR3); (II) SEQ ID NO: 229 (HCDR1), SEQ ID NO: 311 (HCDR2), SEQ ID NO: 348 (HCDR3), SEQ ID NO: 320 (LCDR1), SEQ ID NO: 354 (LCDR2), and SEQ ID NO: 355 (LCDR3); (III) SEQ ID NO: 80 (HCDR1), SEQ ID NO: 313 (HCDR2), SEQ ID NO: 348 (HCDR3), SEQ ID NO: 323 (LCDR1), SEQ ID NO: 324 (LCDR2), and SEQ ID NO: 356 (LCDR3); or (IV) SEQ ID NO: 82 (HCDR1), SEQ ID NO: 314 (HCDR2), SEQ ID NO: 349 (HCDR3), SEQ ID NO: 326 (LCDR1), SEQ ID NO: 324 (LCDR2), and SEQ ID NO: 355 (LCDR3).

In some embodiments of the invention, the target (anti-NPR1) antibody or antigen binding fragment comprises: (a) a heavy chain variable region comprising an amino acid sequence of SEQ ID NO: 201, and a light chain variable region comprising an amino acid sequence of SEQ ID NO: 136; (b) a heavy chain variable region comprising an amino acid sequence of SEQ ID NO: 122, and a light chain variable region comprising an amino acid sequence of SEQ ID NO: 136; (c) a heavy chain variable region comprising an amino acid sequence of SEQ ID NO: 201, and a light chain variable region comprising an amino acid sequence of SEQ ID NO: 128; (d) a heavy chain variable region comprising an amino acid sequence of SEQ ID NO: 122, and a light chain variable region comprising an amino acid sequence of SEQ ID NO: 128; (e) a heavy chain variable region comprising an amino acid sequence of SEQ ID NO: 201, and a light chain variable region comprising an amino acid sequence of SEQ ID NO: 147; (f) a heavy chain variable region comprising an amino acid sequence of SEQ ID NO: 122, and a light chain variable region comprising an amino acid sequence of SEQ ID NO: 147; (g) a heavy chain variable region comprising an amino acid sequence of SEQ ID NO: 201, and a light chain variable region comprising an amino acid sequence of SEQ ID NO: 174; (h) a heavy chain variable region comprising an amino acid sequence of SEQ ID NO: 122, and a light chain variable region comprising an amino acid sequence of SEQ ID NO: 174; (i) a heavy chain variable region comprising an amino acid sequence of SEQ ID NO: 201, and a light chain variable region comprising an amino acid sequence of SEQ ID NO: 180; (j) a heavy chain variable region comprising an amino acid sequence of SEQ ID NO: 122, and a light chain variable region comprising an amino acid sequence of SEQ ID NO: 180; (k) a heavy chain variable region comprising an amino acid sequence of SEQ ID NO: 201, and a light chain variable region comprising an amino acid sequence of SEQ ID NO: 186; (1) a heavy chain variable region comprising an amino acid sequence of SEQ ID NO: 122, and a light chain variable region comprising an amino acid sequence of SEQ ID NO: 186; (m) a heavy chain variable region comprising an amino acid sequence of SEQ ID NO: 103, and a light chain variable region comprising an amino acid sequence of SEQ ID NO: 24; (n) a heavy chain variable region comprising an amino acid sequence of SEQ ID NO: 115, and a light chain variable region comprising an amino acid sequence of SEQ ID NO: 24; (o) a heavy chain variable region comprising an amino acid sequence of SEQ ID NO: 122, and a light chain variable region comprising an amino acid sequence of SEQ ID NO: 48; (p) a heavy chain variable region comprising an amino acid sequence of SEQ ID NO: 37, and a light chain variable region comprising an amino acid sequence of SEQ ID NO: 128; (q) a heavy chain variable region comprising an amino acid sequence of SEQ ID NO: 37, and a light chain variable region comprising an amino acid sequence of SEQ ID NO: 136; (r) a heavy chain variable region comprising an amino acid sequence of SEQ ID NO: 37, and a light chain variable region comprising an amino acid sequence of SEQ ID NO: 147; (s) a heavy chain variable region comprising an amino acid sequence of SEQ ID NO: 154, and a light chain variable region comprising an amino acid sequence of SEQ ID NO: 24; (t) a heavy chain variable region comprising an amino acid sequence of SEQ ID NO: 161, and a light chain variable region comprising an amino acid sequence of SEQ ID NO: 24; (u) a heavy chain variable region comprising an amino acid sequence of SEQ ID NO: 168, and a light chain variable region comprising an amino acid sequence of SEQ ID NO: 24; (v) a heavy chain variable region comprising an amino acid sequence of SEQ ID NO: 37, and a light chain variable region comprising an amino acid sequence of SEQ ID NO: 174; (w) a heavy chain variable region comprising an amino acid sequence of SEQ ID NO: 37, and a light chain variable region comprising an amino acid sequence of SEQ ID NO: 180; (x) a heavy chain variable region comprising an amino acid sequence of SEQ ID NO: 37, and a light chain variable region comprising an amino acid sequence of SEQ ID NO: 186; (y) a heavy chain variable region comprising an amino acid sequence of SEQ ID NO: 193, and a light chain variable region comprising an amino acid sequence of SEQ ID NO: 136; (z) a heavy chain variable region comprising an amino acid sequence of SEQ ID NO: 193, and a light chain variable region comprising an amino acid sequence of SEQ ID NO: 174; (aa) a heavy chain variable region comprising an amino acid sequence of SEQ ID NO: 13, and a light chain variable region comprising an amino acid sequence of SEQ ID NO: 24; (bb) a heavy chain variable region comprising an amino acid sequence of SEQ ID NO: 37, and a light chain variable region comprising an amino acid sequence of SEQ ID NO: 48; (cc) a heavy chain variable region comprising an amino acid sequence of SEQ ID NO: 374, and a light chain variable region comprising an amino acid sequence of SEQ ID NO: 244; (dd) a heavy chain variable region comprising an amino acid sequence of SEQ ID NO: 385, and a light chain variable region comprising an amino acid sequence of SEQ ID NO: 244; (ee) a heavy chain variable region comprising an amino acid sequence of SEQ ID NO: 233, and a light chain variable region comprising an amino acid sequence of SEQ ID NO: 244; (ff) a heavy chain variable region comprising an amino acid sequence of SEQ ID NO: 278, and a light chain variable region comprising an amino acid sequence of SEQ ID NO: 287; or (gg) a heavy chain variable region comprising an amino acid sequence of SEQ ID NO: 300, and a light chain variable region comprising an amino acid sequence of SEQ ID NO: 306.

In some embodiments of the invention, the target (anti-NPR1) antibody or antigen binding fragment comprises: (a) a heavy chain variable region comprising an amino acid sequence of SEQ ID NO: 61, and a light chain variable region comprising an amino acid sequence of SEQ ID NO: 72; (b) a heavy chain variable region comprising an amino acid sequence of SEQ ID NO: 85, and a light chain variable region comprising an amino acid sequence of SEQ ID NO: 96; (c) a heavy chain variable region comprising an amino acid sequence of SEQ ID NO: 350, and a light chain variable region comprising an amino acid sequence of SEQ ID NO: 363; (d) a heavy chain variable region comprising an amino acid sequence of SEQ ID NO: 392, and a light chain variable region comprising an amino acid sequence of SEQ ID NO: 344; (e) a heavy chain variable region comprising an amino acid sequence of SEQ ID NO: 414, and a light chain variable region comprising an amino acid sequence of SEQ ID NO: 344; (f) a heavy chain variable region comprising an amino acid sequence of SEQ ID NO: 316, and a light chain variable region comprising an amino acid sequence of SEQ ID NO: 327; (g) a heavy chain variable region comprising an amino acid sequence of SEQ ID NO: 333, and a light chain variable region comprising an amino acid sequence of SEQ ID NO: 344; or (h) a heavy chain variable region comprising an amino acid sequence of SEQ ID NO: 350, and a light chain variable region comprising an amino acid sequence of SEQ ID NO: 357.

In some embodiments of the invention, the target (anti-NPR1) antibody or antigen binding fragment comprises: (a) a heavy chain comprising an amino acid sequence of SEQ ID NO: 203, and a light chain comprising an amino acid sequence of SEQ ID NO: 138; (b) a heavy chain comprising an amino acid sequence of SEQ ID NO: 208, and a light chain comprising an amino acid sequence of SEQ ID NO: 138; (c) a heavy chain comprising an amino acid sequence of SEQ ID NO: 203, and a light chain comprising an amino acid sequence of SEQ ID NO: 130; (d) a heavy chain comprising an amino acid sequence of SEQ ID NO: 208, and a light chain comprising an amino acid sequence of SEQ ID NO: 130; (e) a heavy chain comprising an amino acid sequence of SEQ ID NO: 203, and a light chain comprising an amino acid sequence of SEQ ID NO: 149; (f) a heavy chain comprising an amino acid sequence of SEQ ID NO: 208, and a light chain comprising an amino acid sequence of SEQ ID NO: 149; (g) a heavy chain comprising an amino acid sequence of SEQ ID NO: 203, and a light chain comprising an amino acid sequence of SEQ ID NO: 176; (h) a heavy chain comprising an amino acid sequence of SEQ ID NO: 208, and a light chain comprising an amino acid sequence of SEQ ID NO: 176; (i) a heavy chain comprising an amino acid sequence of SEQ ID NO: 203, and a light chain comprising an amino acid sequence of SEQ ID NO: 182; (j) a heavy chain comprising an amino acid sequence of SEQ ID NO: 208, and a light chain comprising an amino acid sequence of SEQ ID NO: 182; (k) a heavy chain comprising an amino acid sequence of SEQ ID NO: 203, and a light chain comprising an amino acid sequence of SEQ ID NO: 188; (1) a heavy chain comprising an amino acid sequence of SEQ ID NO: 208, and a light chain comprising an amino acid sequence of SEQ ID NO: 188; (m) a heavy chain comprising an amino acid sequence of SEQ ID NO: 105, and a light chain comprising an amino acid sequence of SEQ ID NO: 26; (n) a heavy chain comprising an amino acid sequence of SEQ ID NO: 108, and a light chain comprising an amino acid sequence of SEQ ID NO: 26; (o) a heavy chain comprising an amino acid sequence of SEQ ID NO: 117, and a light chain comprising an amino acid sequence of SEQ ID NO: 26; (p) a heavy chain comprising an amino acid sequence of SEQ ID NO: 124, and a light chain comprising an amino acid sequence of SEQ ID NO: 50; (q) a heavy chain comprising an amino acid sequence of SEQ ID NO: 39, and a light chain comprising an amino acid sequence of SEQ ID NO: 130; (r) a heavy chain comprising an amino acid sequence of SEQ ID NO: 39, and a light chain comprising an amino acid sequence of SEQ ID NO: 138; (s) a heavy chain comprising an amino acid sequence of SEQ ID NO: 141, and a light chain comprising an amino acid sequence of SEQ ID NO: 138; (t) a heavy chain comprising an amino acid sequence of SEQ ID NO: 39, and a light chain comprising an amino acid sequence of SEQ ID NO: 149; (u) a heavy chain comprising an amino acid sequence of SEQ ID NO: 156, and a light chain comprising an amino acid sequence of SEQ ID NO: 26; (v) a heavy chain comprising an amino acid sequence of SEQ ID NO: 159, and a light chain comprising an amino acid sequence of SEQ ID NO: 26; (w) a heavy chain comprising an amino acid sequence of SEQ ID NO: 163, and a light chain comprising an amino acid sequence of SEQ ID NO: 26; (x) a heavy chain comprising an amino acid sequence of SEQ ID NO: 170, and a light chain comprising an amino acid sequence of SEQ ID NO: 26; (y) a heavy chain comprising an amino acid sequence of SEQ ID NO: 39, and a light chain comprising an amino acid sequence of SEQ ID NO: 176; (z) a heavy chain comprising an amino acid sequence of SEQ ID NO: 39, and a light chain comprising an amino acid sequence of SEQ ID NO: 182; (aa) a heavy chain comprising an amino acid sequence of SEQ ID NO: 39, and a light chain comprising an amino acid sequence of SEQ ID NO: 188; (bb) a heavy chain comprising an amino acid sequence of SEQ ID NO: 195, and a light chain comprising an amino acid sequence of SEQ ID NO: 138; (cc) a heavy chain comprising an amino acid sequence of SEQ ID NO: 195, and a light chain comprising an amino acid sequence of SEQ ID NO: 176; (dd) a heavy chain comprising an amino acid sequence of SEQ ID NO: 15, and a light chain comprising an amino acid sequence of SEQ ID NO: 26; (ee) a heavy chain comprising an amino acid sequence of SEQ ID NO: 39, and a light chain comprising an amino acid sequence of SEQ ID NO: 50; (ff) a heavy chain comprising an amino acid sequence of SEQ ID NO: 376, and a light chain comprising an amino acid sequence of SEQ ID NO: 246; (gg) a heavy chain comprising an amino acid sequence of SEQ ID NO: 387, and a light chain comprising an amino acid sequence of SEQ ID NO: 246; (hh) a heavy chain comprising an amino acid sequence of SEQ ID NO: 235, and a light chain comprising an amino acid sequence of SEQ ID NO: 246; (ii) a heavy chain comprising an amino acid sequence of SEQ ID NO: 280, and a light chain comprising an amino acid sequence of SEQ ID NO: 289; or (jj) a heavy chain comprising an amino acid sequence of SEQ ID NO: 302, and a light chain comprising an amino acid sequence of SEQ ID NO: 308.

In some embodiments of the invention, the target (anti-NPR1) antibody or antigen binding fragment comprises: (a) a heavy chain comprising an amino acid sequence of SEQ ID NO: 63, and a light chain comprising an amino acid sequence of SEQ ID NO: 74; (b) a heavy chain comprising an amino acid sequence of SEQ ID NO: 87, and a light chain comprising an amino acid sequence of SEQ ID NO: 98; (c) a heavy chain comprising an amino acid sequence of SEQ ID NO: 352, and a light chain comprising an amino acid sequence of SEQ ID NO: 365; (d) a heavy chain comprising an amino acid sequence of SEQ ID NO: 394, and a light chain comprising an amino acid sequence of SEQ ID NO: 346; (e) a heavy chain comprising an amino acid sequence of SEQ ID NO: 416, and a light chain comprising an amino acid sequence of SEQ ID NO: 346; (f) a heavy chain comprising an amino acid sequence of SEQ ID NO: 318, and a light chain comprising an amino acid sequence of SEQ ID NO: 329; (g) a heavy chain comprising an amino acid sequence of SEQ ID NO: 335, and a light chain comprising an amino acid sequence of SEQ ID NO: 346; or (h) a heavy chain comprising an amino acid sequence of SEQ ID NO: 352, and a light chain comprising an amino acid sequence of SEQ ID NO: 359.

In some embodiments of the invention, the target (anti-NPR1) antibody or antigen binding fragment is an antigen binding fragment selected from the group consisting of a Fab, Fab′, F(ab′)₂, Fv, single domain antibody (dAb), and a single chain variable fragment (scFv). In some embodiments of the invention, the target (anti-NPR1) antibody or antigen binding fragment is an antigen binding fragment selected from the group consisting of a Fab, Fab′, Fv, single domain antibody (dAb), and a single chain variable fragment (scFv).

In some embodiments of the invention, the target (anti-NPR1) antibody or antigen binding fragment is monoclonal. In some embodiments of the invention, the target (anti-NPR1) antibody or antigen binding fragment is fully human. In some embodiments of the invention, the target (anti-NPR1) antibody or antigen binding fragment is an IgG antibody. In some embodiments of the invention, the target (anti-NPR1) antibody or antigen binding fragment is an IgG1 antibody. In some embodiments of the invention, the target (anti-NPR1) antibody or antigen binding fragment is an IgG1 antibody having a kappa light chain. In some embodiments of the invention, the target (anti-NPR1) antibody or antigen binding fragment is a fully human antibody of the IgG1 isotype and has a kappa light chain.

In some embodiments of the invention, the target (anti-NPR1) antibody or antigen binding fragment additionally has mutations in the Fc region according to the EU index of Kabat, wherein the mutations comprise at least D265A and P329A.

In some embodiments of the invention, the target (anti-NPR1) antibody or antigen binding fragment additionally has mutations in the Fc region according to the EU index of Kabat, wherein the mutations comprise at least L234A and L235A.

In some embodiments of the invention, the target (anti-NPR1) antibody or antigen binding fragment is therapeutic.

In some embodiments of the invention, the target (anti-NPR1) antibody or antigen binding fragment binds to the same epitope on human NPR1 as any of the antibodies or antigen binding fragments or groups defined herein (e.g., XX16). In some embodiments of the invention, the target (anti-NPR1) antibody or antigen binding fragment competes for binding to human NPR1 with any of the antibodies or antigen binding fragments or groups defined herein (e.g., XX16).

In some embodiments of the invention, the binding agent is an antibody or an antigen-binding fragment thereof. In some embodiments of the invention, the binding agent is a monoclonal human antibody. In some embodiments of the invention, the binding agent is an antigen binding fragment selected from the group consisting of a Fab, Fab′, F(ab′)₂, Fv, single domain antibody (dAb), and a single chain variable fragment (scFv).

In some embodiments of the invention, the binding agent is for use in the treatment of a disorder or disease associated with administration of a natriuretic peptide receptor agonist in a subject in need of such treatment. In some embodiments of the invention, the binding agent is for use in the treatment of a disorder or disease associated with administration of the target antibody or antigen binding fragment thereof in a subject in need of such treatment. In some embodiments of the invention, the binding agent reverses and/or inhibits the agonism of the NPR1 pathway caused by the target antibody or antigen binding fragment thereof in a subject in need of such treatment. In some embodiments of the invention, the binding agent reverses and/or inhibits the generation of cGMP caused by the target antibody or antigen binding fragment thereof in a subject in need of such treatment. In some embodiments of the invention, the disease or disorder is hypotension.

In one aspect of the invention, provided herein is a pharmaceutical composition for use as a medicament reversing the hypotensive effect of an anti-NPR1 target antibody or antigen-binding fragment thereof in a subject being treated with the anti-NPR1 target antibody or antigen-binding fragment thereof, wherein the pharmaceutical composition comprises an effective amount of any one of the binding agents described herein.

In one aspect of the invention, provided herein is a method for reversing the hypotensive effect of an anti-NPR1 target antibody or antigen-binding fragment thereof in a subject being treated with the anti-NPR1 target antibody or antigen-binding fragment thereof, comprising administering an effective amount of any one of the binding agents described herein to a subject in need thereof.

In one aspect of the invention, provided herein are binding agents for use in reversing the hypotensive effect of an anti-NPR1 target antibody or antigen-binding fragment thereof in a subject being treated with the anti-NPR1 target antibody or antigen-binding fragment thereof, comprising administering an effective amount of any one of the binding agents described herein to a subject in need thereof.

In one aspect of the invention, provided herein are binding agents for use in reversing the effect of an anti-NPR1 target antibody or antigen-binding fragment thereof on cGMP levels in a subject being treated with the anti-NPR1 target antibody or antigen-binding fragment thereof, comprising administering an effective amount of any one of the binding agents described herein to a subject in need thereof.

In some embodiments of the invention, the anti-NPR1 target antibody or antigen-binding fragment thereof competes with, or binds to the same epitope as, a reference anti-NPR1 target antibody or antigen-binding fragment thereof comprising a heavy chain comprising the amino acid sequence of SEQ ID NO: 208 and a light chain comprising the amino acid sequence of SEQ ID NO: 138.

In some embodiments of the invention, the method further comprises additionally administering one of the following to the subject: (i) fluid infusion; and/or (ii) one or more vasopressors. In some embodiments of the invention, the fluid infusion is a crystalloid IV fluid infusion. In some embodiments of the invention, the fluid infusion is a saline infusion. In some embodiments of the invention, the fluid infusion is an infusion of one or more of the following: lactated Ringer's solution, hypertonic saline, and isotonic saline. In some embodiments of the invention, the one or more vasopressors is selected from phenylephrine, norepinephrine, epinephrine, and vasopressin. In some embodiments of the invention, the vasopressor is arg-vasopressin. In some embodiments of the invention, the vasopressor is antiotensin II.

In some embodiments of the invention, the method additionally comprises halting treatment with an additional therapeutic agent. In some embodiments of the invention, the method additionally comprises halting treatment with an anti-hypertensive agent, a beta blocker, and/or a diuretic.

In some embodiments of the invention, the method additionally comprises halting treatment with an additional therapeutic agent, and the additional therapeutic agent is selected from an ACE (angiotensin-converting-enzyme) inhibitor, an angiotensin receptor blocker (ARB), a neprilysin inhibitor, a beta blocker, a diuretic, a calcium channel blocker, a cardiac glycoside, a sodium-glucose co-transporter 2 inhibitor (SGLT2i), and/or an angiotensin receptor-neprilysin inhibitor (ARNi). In some embodiments of the invention, the method additionally comprises halting treatment with an additional therapeutic agent, and the additional therapeutic agent is selected from enalapril, benazepril, captopril, fosinopril, lisinopril, moexipril, perindopril, quinapril, ramipril, trandolapril, valsartan, azilsartan, candesartan, eprosartan, irbesartan, losartan, olmesartan, telmisartan, sacubitril, bisoprolol, carvedilol, propanolol, metoprolol, metoprolol tartrate, metoprolol succinate, thiazide diuretics, loop diuretics, potassium-sparing diuretics, amlodipine, clevidipine, diltiazem, felodipine, isradipine, nicardipine, nifedipine, nisoldipine, verapamil, a digitalis glycoside, canagliflozin, dapagliflozin, empagliflozin, ertugliflozin, chlorothiazide, chlorthalidone, hydrochlorothiazide, indapamide, metolazone, bumetanide, ethacrynic acid, furosemide, torsemide, amiloride, eplerenone, spironolactonem, triamterene, digoxin, and combinations thereof.

In some embodiments of the invention, the subject has or is at risk of developing a cardiovascular disorder. In some embodiments of the invention, the cardiovascular disorder is selected from: hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, and myocardial infarction (MI). In some embodiments of the invention, the subject has or is at risk of developing heart failure, hypertrophic cardiomyopathy (HCM), hypertension, preeclampsia, asthma, glaucoma, a kidney disorder, and/or cytokine release syndrome. In some embodiments of the invention, the heart failure is selected from a heart failure with reduced ejection fraction (HFrEF), heart failure with preserved ejection fraction (HFpEF), heart failure after acute myocardial infarct, or acute decompensated heart failure. In some embodiments of the invention, the hypertension is selected from resistant hypertension, hypertensive heart disease, pulmonary hypertension, pulmonary arterial hypertension, and isolated systolic hypertension.

In one aspect of the invention, provided herein is an anti-idiotype antibody that specifically binds to an anti-NPR1 antibody or antigen binding fragment thereof wherein the anti-NPR1 antibody or antigen binding fragment thereof is XX16 or comprises the heavy chain variable region CDRs and light chain variable region CDRs of XX16. In one aspect of the invention, provided herein is an anti-idiotype antibody that specifically binds to an anti-NPR1 antibody or antigen binding fragment thereof, wherein the target antibody or antigen binding fragment is ANP non-competitive. In one aspect of the invention, provided herein is an anti-idiotype antibody that specifically binds to an anti-NPR1 antibody or antigen binding fragment thereof, wherein the target antibody or antigen binding fragment is ANP competitive. In one embodiment of the invention, the anti-NPR1 antibody or antigen binding fragment thereof binds to an epitope within amino acids 99-133 of SEQ ID NO: 1. In one embodiment of the invention, the anti-NPR1 antibody or antigen binding fragment thereof binds to a conformational epitope of human NPR1, and wherein the conformational epitope comprises at least one amino acid residue within each of (i) amino acids 99-103 of SEQ ID NO: 1, (ii) 105-111 of SEQ ID NO: 1, (iii) 131-134 of SEQ ID NO: 1, and additionally binds to amino acid 375 and/or 378 of SEQ ID NO: 1. In one embodiment of the invention, the anti-NPR1 antibody or antigen binding fragment thereof binds to an epitope within 188-219 of SEQ ID NO: 1. In one embodiment of the invention, the anti-NPR1 antibody or antigen binding fragment thereof binds to a conformational epitope of human NPR1, and wherein the conformational epitope comprises at least one amino acid residue within each of (i) amino acids 188-198 of SEQ ID NO: 1, (ii) 201-208 of SEQ ID NO: 1, (iii) 215-238 of SEQ ID NO: 1, and (iv) 294-297 of SEQ ID NO: 1, optionally wherein the antibody or antigen binding fragment thereof binds to at least amino acids 188, 192, 194, 197, 201, 208, and 219 of SEQ ID NO: 1. In one aspect of the invention, provided herein is an anti-idiotype antibody that specifically binds to an anti-NPR1 antibody, wherein the anti-NPR1 antibody or antigen binding fragment thereof binds to the same epitope of NPR1 as XX16, or competes for binding to NPR1 with XX16. In one embodiment of the invention, the anti-NPR1 antibody or antigen binding fragment thereof from binding to NPR1. In one embodiment of the invention, the anti-idiotype antibody of any one of claims 40-48, which reverses and/or inhibits the agonism of the NPR1 pathway caused by the anti-NPR1 antibody or antigen binding fragment thereof in a subject in need of such treatment. In one embodiment of the invention, the binding agent reverses and/or inhibits the generation of cGMP caused by the anti-NPR1 antibody or antigen binding fragment thereof in a subject in need of such treatment. In one embodiment of the invention, the anti-idiotype antibody reverses or inhibits the hypotensive effects of the anti-NPR1 antibody.

In one aspect of the invention, provided herein is a method of managing hypotension risk in subject treated or administered an anti-NPR1 antibody or antigen binding fragment thereof, comprising the step of administering to the subject in need thereof, an anti-idiotype antibody of the anti-NPR1 antibody or antigen binding fragment thereof, wherein the anti-idiotype specifically binds to the anti-NPR1 antibody or antigen binding fragment thereof and blocks the anti-NPR1 antibody or antigen binding fragment thereof from binding to NPR1, and wherein the anti-NPR1 antibody or antigen binding fragment thereof is XX16 or comprises the heavy chain variable region CDRs and light chain variable region CDRs of XX16. In one aspect of the invention, provided herein is a method of managing hypotension risk in subject treated or administered an anti-NPR1 antibody or antigen binding fragment thereof, comprising the step of administering to the subject in need thereof, an anti-idiotype antibody of the anti-NPR1 antibody or antigen binding fragment thereof, wherein the anti-idiotype specifically binds to the anti-NPR1 antibody or antigen binding fragment thereof and blocks the anti-NPR1 antibody or antigen binding fragment thereof from binding to NPR1, and wherein the anti-NPR1 antibody binds to the same epitope of NPR1 as XX16, or competes for binding to NPR1 with XX16. In one embodiment of the invention, the anti-NPR1 antibody or antigen binding fragment thereof binds to a conformational epitope of human NPR1, and wherein the conformational epitope comprises at least one amino acid residue within each of (i) amino acids 99-103 of SEQ ID NO: 1, (ii) 105-111 of SEQ ID NO: 1, (iii) 131-134 of SEQ ID NO: 1, and additionally binds to amino acid 375 and/or 378 of SEQ ID NO: 1. In one embodiment of the invention, the anti-NPR1 antibody or antigen binding fragment thereof binds to a conformational epitope of human NPR1, and wherein the conformational epitope comprises at least one amino acid residue within each of (i) amino acids 188-198 of SEQ ID NO: 1, (ii) 201-208 of SEQ ID NO: 1, (iii) 215-238 of SEQ ID NO: 1, and (iv) 294-297 of SEQ ID NO: 1, optionally wherein the antibody or antigen binding fragment thereof binds to at least amino acids 188, 192, 194, 197, 201, 208, and 219 of SEQ ID NO: 1. In one embodiment of the invention, the anti-idiotype antibody or antigen binding fragment thereof reverses the hypotensive effects of the anti-NPR1 antibody.

In one aspect of the invention, provided herein is a method of managing low blood pressure in subject treated or administered an anti-NPR1 antibody or antigen binding fragment thereof, comprising the step of administering to the subject in need thereof, an anti-idiotype antibody of the anti-NPR1 antibody or antigen binding fragment thereof, wherein the anti-idiotype specifically binds to the anti-NPR1 antibody or antigen binding fragment thereof and blocks the anti-NPR1 antibody or antigen binding fragment thereof from binding to NPR1, and wherein the anti-NPR1 antibody or antigen binding fragment thereof is XX16 or comprises the heavy chain variable region CDRs and light chain variable region CDRs of XX16. In one aspect of the invention, provided herein is a method of managing low blood pressure in subject treated or administered an anti-NPR1 antibody or antigen binding fragment thereof, comprising the step of administering to the subject in need thereof, an anti-idiotype antibody of the anti-NPR1 antibody or antigen binding fragment thereof, wherein the anti-idiotype specifically binds to the anti-NPR1 antibody or antigen binding fragment thereof and blocks the anti-NPR1 antibody or antigen binding fragment thereof from binding to NPR1, and wherein the anti-NPR1 antibody binds to the same epitope of NPR1 as XX16, or competes for binding to NPR1 with XX16. In one embodiment of the invention, the anti-NPR1 antibody or antigen binding fragment thereof binds to a conformational epitope of human NPR1, and wherein the conformational epitope comprises at least one amino acid residue within each of (i) amino acids 99-103 of SEQ ID NO: 1, (ii) 105-111 of SEQ ID NO: 1, (iii) 131-134 of SEQ ID NO: 1, and additionally binds to amino acid 375 and/or 378 of SEQ ID NO: 1.

In one embodiment of the invention, the anti-NPR1 antibody or antigen binding fragment thereof binds to a conformational epitope of human NPR1, and wherein the conformational epitope comprises at least one amino acid residue within each of (i) amino acids 188-198 of SEQ ID NO: 1, (ii) 201-208 of SEQ ID NO: 1, (iii) 215-238 of SEQ ID NO: 1, and (iv) 294-297 of SEQ ID NO: 1, optionally wherein the antibody or antigen binding fragment thereof binds to at least amino acids 188, 192, 194, 197, 201, 208, and 219 of SEQ ID NO: 1. In one embodiment of the invention, the anti-idiotype antibody or antigen binding fragment thereof reverses the low blood pressure of the subject.

In one aspect of the invention, provided herein is a method of reversing and/or inhibiting the agonism of the NPR1 pathway caused by an anti-NPR1 antibody or antigen binding fragment thereof in a subject in need of such treatment, comprising the step of administering to the subject in need thereof, an anti-idiotype antibody of the anti-NPR1 antibody or antigen binding fragment thereof, wherein the anti-idiotype specifically binds to the anti-NPR1 antibody or antigen binding fragment thereof and blocks the anti-NPR1 antibody or antigen binding fragment thereof from binding to NPR1, and wherein the anti-NPR1 antibody or antigen binding fragment thereof is XX16 or comprises the heavy chain variable region CDRs and light chain variable region CDRs of XX16. In one aspect of the invention, provided herein is a method of reversing and/or inhibiting the agonism of the NPR1 pathway caused by an anti-NPR1 antibody or antigen binding fragment thereof in a subject in need of such treatment, comprising the step of administering to the subject in need thereof, an anti-idiotype antibody of the anti-NPR1 antibody or antigen binding fragment thereof, wherein the anti-idiotype specifically binds to the anti-NPR1 antibody or antigen binding fragment thereof and blocks the anti-NPR1 antibody or antigen binding fragment thereof from binding to NPR1, and wherein the anti-NPR1 antibody binds to the same epitope of NPR1 as XX16, or competes for binding to NPR1 with XX16. In one embodiment of the invention, the anti-NPR1 antibody or antigen binding fragment thereof binds to a conformational epitope of human NPR1, and wherein the conformational epitope comprises at least one amino acid residue within each of (i) amino acids 99-103 of SEQ ID NO: 1, (ii) 105-111 of SEQ ID NO: 1, (iii) 131-134 of SEQ ID NO: 1, and additionally binds to amino acid 375 and/or 378 of SEQ ID NO: 1. In one embodiment of the invention, the anti-NPR1 antibody or antigen binding fragment thereof binds to a conformational epitope of human NPR1, and wherein the conformational epitope comprises at least one amino acid residue within each of (i) amino acids 188-198 of SEQ ID NO: 1, (ii) 201-208 of SEQ ID NO: 1, (iii) 215-238 of SEQ ID NO: 1, and (iv) 294-297 of SEQ ID NO: 1, optionally wherein the antibody or antigen binding fragment thereof binds to at least amino acids 188, 192, 194, 197, 201, 208, and 219 of SEQ ID NO: 1. In one embodiment of the invention, the anti-idiotype antibody or antigen binding fragment thereof reverses the hypotensive effects of the anti-NPR1 antibody.

In one aspect of the invention, provided herein is a In one aspect of the invention, provided herein is a method of reversing and/or inhibiting the generation of cGMP caused by an anti-NPR1 antibody or antigen binding fragment thereof in a subject in need of such treatment, comprising the step of administering to the subject in need thereof, an anti-idiotype antibody of the anti-NPR1 antibody or antigen binding fragment thereof, wherein the anti-idiotype specifically binds to the anti-NPR1 antibody or antigen binding fragment thereof and blocks the anti-NPR1 antibody or antigen binding fragment thereof from binding to NPR1, and wherein the anti-NPR1 antibody or antigen binding fragment thereof is XX16 or comprises the heavy chain variable region CDRs and light chain variable region CDRs of XX16. In one aspect of the invention, provided herein is a method of reversing and/or inhibiting the generation of cGMP caused by an anti-NPR1 antibody or antigen binding fragment thereof in a subject in need of such treatment, comprising the step of administering to the subject in need thereof, an anti-idiotype antibody of the anti-NPR1 antibody or antigen binding fragment thereof, wherein the anti-idiotype specifically binds to the anti-NPR1 antibody or antigen binding fragment thereof and blocks the anti-NPR1 antibody or antigen binding fragment thereof from binding to NPR1, and wherein the anti-NPR1 antibody binds to the same epitope of NPR1 as XX16, or competes for binding to NPR1 with XX16. In one embodiment of the invention, the anti-NPR1 antibody or antigen binding fragment thereof binds to a conformational epitope of human NPR1, and wherein the conformational epitope comprises at least one amino acid residue within each of (i) amino acids 99-103 of SEQ ID NO: 1, (ii) 105-111 of SEQ ID NO: 1, (iii) 131-134 of SEQ ID NO: 1, and additionally binds to amino acid 375 and/or 378 of SEQ ID NO: 1. In one embodiment of the invention, the anti-NPR1 antibody or antigen binding fragment thereof binds to a conformational epitope of human NPR1, and wherein the conformational epitope comprises at least one amino acid residue within each of (i) amino acids 188-198 of SEQ ID NO: 1, (ii) 201-208 of SEQ ID NO: 1, (iii) 215-238 of SEQ ID NO: 1, and (iv) 294-297 of SEQ ID NO: 1, optionally wherein the antibody or antigen binding fragment thereof binds to at least amino acids 188, 192, 194, 197, 201, 208, and 219 of SEQ ID NO: 1. In one embodiment of the invention, the anti-idiotype antibody or antigen binding fragment thereof reverses the hypotensive effects of the anti-NPR1 antibody.

In one aspect of the invention, provided herein is an isolated nucleic acid or nucleic acids encoding the amino acid sequence of any of the antibodies or antigen binding fragments or groups defined herein. In one aspect of the invention, provided herein is a vector comprising the isolated nucleic acid(s). In one aspect of the invention, provided herein is a host cell comprising the isolated nucleic acid(s) or the vector.

In one aspect of the invention, provided herein is a method of producing any of the antibodies or antigen binding fragments described herein, comprising culturing the host cell described herein under conditions suitable to produce the antibody or antigen binding fragment. In some embodiments of the invention, the method additionally comprises purification of the antibody or antigen binding fragment.

In one aspect of the invention, provided herein is pharmaceutical composition comprising a purified antibody or antigen binding fragment produced by the method described herein and a pharmaceutically acceptable carrier.

In one aspect of the invention, provided herein is pharmaceutical composition comprising any of the antibodies or antigen binding fragments described herein and a pharmaceutically acceptable carrier.

In some embodiments of the invention, the composition further comprises an additional therapeutic agent.

### BRIEF DESCRIPTION OF THE FIGURES

FIG. 1 is a set of graphs displaying the results of antibody candidates WW01, WW02, WW03, WW04, and WW06 binding to the following antigens (ELISA analysis): human NPR1, constitutively active human NPR1 mutant (W74R), rat NPR1, and human NPR3 (counter target) both in the absence of and presence of a 250 fold molar excess of ANP.

FIG. 2 is a set of graphs displaying the results of flow cytometry analysis of antibody candidates WW01, WW02, WW03, WW04, and WW06 for binding to human NPR1 expressing CHO-K1 cells in the absence and presence of a saturating concentration of ANP and on parental CHO-K1 cells.

FIG. 3 is a graphical representation of a Fluorescence Resonance Energy Transfer (FRET)-based assay in which the NPR1-specific antibodies competed with ANP for binding to NPR1. In this FRET based assay, Eu-labeled Streptavidin (for measurement of IgGs) or Eu-labeled anti-hFc antibody (for measurement of FabCys) was used as an energy donor, while Cy5-labeled ANP was used as an acceptor.

FIG. 4 is a set of graphs displaying the results of ANP competition analyses of candidates WW01, WW02, WW03, WW04, and WW06 using the FRET-based assay depicted in FIG. 3.

FIG. 5 is a set of graphs depicting the results of functional activity analyses of candidates WW01, WW02, WW03, WW04, and WW06 in a cellular cGMP production assay using human NPR1 expressing CHO-K1 cells. Results represent the cellular production of cGMP [nM] in the absence or presence of 0.075 nM ANP.

FIG. 6 is a set of graphs depicting the results of functional activity analyses of candidate WW06 in IgG or FabCys format in a cellular cGMP production assay using human NPR1 expressing CHO-K1 cells. Results represent the cellular production of cGMP [nM] in the absence or presence of 0.075 nM ANP.

FIG. 7 demonstrates the results of SET screening (hNPR1 affinity) of 82 HCDR2 or LCDR3 unique improved HuCAL® derivatives based on the parental antibody.

FIG. 8 demonstrates the results of SET screening (hNPR1-ANP complex affinity) of 82 HCDR2 or LCDR3 unique improved HuCAL® derivatives based on the parental antibody.

FIG. 9 is a set of graphs displaying the results of antibody candidates XX01-XX08, XX10, and XX12 binding to the following antigens (ELISA analysis): human NPR1, constitutively active human NPR1 mutant (W74R), rat NPR1, and human NPR3 (counter target) in the absence of or in the presences of a 250 fold molar excess of ANP.

FIG. 10 is a set of graphs displaying the results of flow cytometry analysis of antibody candidates XX01-XX08, XX10, and XX12 for binding to human NPR1 expressing CHO-K1 cells in the absence or presence of a saturating concentration of ANP and on parental CHO-K1 cells.

FIG. 11 is a set of graphs displaying the results of ANP competition analyses of candidates XX01-XX07, XX10, and XX12 using the FRET-based assay depicted in FIG. 3.

FIG. 12 is a set of graphs depicting the results of functional activity analyses of candidates XX01-XX08, XX10, and XX12 in a cellular cGMP production assay using human NPR1 expressing CHO-K1 cells. Results represent the cellular production of cGMP [nM] in the absence or presence of 0.075 nM ANP.

FIG. 13 is a set of graphs depicting the results of functional activity analyses of candidates XX06 and XX16 alongside natural ligand ANP in a cellular cGMP production assay using human NPR1 expressing CHO-K1 cells. Results represent the cellular production of cGMP [nM] in the absence or presence of 0.075 nM ANP.

FIG. 14 is a graph depicting the results of functional activity analyses of candidate XX16 alongside natural ligand ANP in a cellular cGMP production assay using human NPR1 expressing CHO-K1 cells. Results represent the cellular production of cGMP [nM] as a function of either ANP or XX16.

FIG. 15 is a graphical representation of the crystal structure of Fab06 (the WW06 antibody in Fab format) in complex with hNPR1 extracellular domain.

FIG. 16 is a graphical representation of the conformation of the hNPR1 extracellular domain as it would be in complex with Fab06. The Fab06 were removed from this representation to more clearly reveal the conformation of hNPR1 induced by Fab binding. W74R is shown in space filling.

FIG. 17 is a graphical representation of the crystal structure of hNPR1 extracellular domain in complex with ANP (left) and the crystal structure of Fab16 (the XX16 antibody in Fab format) in complex with hNPR1 extracellular domain (right) with W74 shown in space filling.

FIG. 18 is a set of graphs depicting the results of functional activity analyses of candidate WW06 in a cellular cGMP production assay using when tested on CHO-K1 cells expressing hNPR1 W74R/C232T (constitutively active mutant; panel A) compared to WT hNPR1 (panel B).

FIG. 19 is a set of graphs displaying the results of antibody candidates YY01-YY07 binding to the following antigens (ELISA analysis): human NPR1, constitutively active human NPR1 mutant (W74R), rat NPR1, and human NPR3 (counter target) in the absence of or presence of a 250 fold molar excess of ANP.

FIG. 20 is a set of graphs displaying the results of flow cytometry analysis of antibody candidates YY01-YY07 for binding to human NPR1 expressing CHO-K1 cells in the absence or presence of a saturating concentration of ANP and on parental CHO K1 cells.

FIG. 21 is a set of graphs displaying the results of ANP competition analyses of candidates YY01-YY07 using the FRET-based assay depicted in FIG. 3.

FIG. 22 is a set of graphs depicting the results of functional activity analyses of candidates YY01-YY07 in a cellular cGMP production assay using human NPR1 expressing CHO-K1 cells. Results represent the cellular production of cGMP [nM] in the absence or presence of 0.075 nM ANP.

FIG. 23 is a set of graphs depicting the results of functional activity analyses of candidates YY05 and YY07 in IgG or FabCys format in a cellular cGMP production assay using human NPR1 expressing CHO-K1 cells. Results represent the cellular production of cGMP [nM] in the absence or presence of 0.075 nM ANP.

FIG. 24 demonstrates the results of SET screening (hNPR1 affinity) of 112 HCDR1/2 or LCDR3 unique improved Ylanthia® derivatives based on the parental antibody.

FIG. 25 demonstrates the results of SET screening (hNPR1-ANP complex affinity) of 112 HCDR1/2 or LCDR3 unique improved Ylanthia® derivatives based on the parental antibody.

FIG. 26 is a set of graphs representing the plasma cGMP (nM) concentration over time in hNPR1 Tg mice which were intravenously administered 1 mg/kg, 3 mg/kg, or 10 mg/kg of the WW06 Fab.

FIG. 27 is a graph representing the concentration of antibody over time in hNPR1 Tg mice which were intravenously administered 1 mg/kg, 3 mg/kg, or 10 mg/kg of the WW06 Fab.

FIG. 28 is a set of graphs demonstrating the results of subcutaneous administration of vehicle, 0.3 mg/kg XX16, or 3 mg/kg XX16 once every two weeks in wild type or ANP knockout (ANP KO) mice on heart weight/body weight ratio (left) and plasma NT-proBNP levels (pg/mL; right). Measurements were taken two weeks after the second administration.

FIG. 29 is a set of graphs demonstrating the results of a single intravenous administration of vehicle, 0.3 mg/kg XX16, or 1 mg/kg XX16 on blood pressure (mean arterial pressure; left) and urinary flow rate (right) in hypertensive rats (spontaneous hypertensive rat stroke prone, SHRsp). Measurements were taken three hours after the intravenous administration.

FIG. 30 is a set of graphs demonstrating the results of a single subcutaneous administration of 0.1, 0.3, 1, 3, 10, or 30 mg/kg XX16 in telemetry implanted normal rats on mean arterial pressure (MAP; top) and plasma cGMP (bottom) over time.

FIG. 31 is a series of graphs showing the results from a cGMP HTRF assay. XX16 at a constant concentration of 10 nM was incubated with increasing concentrations of anti-XX16 Fabs and dose response curves for the generation of cGMP were generated as described in Example 23. IC₅₀ values were calculated by non-linear fitting of the dose response curves using a variable slope (four parameter) fit equation.

FIG. 32 is a set of graphs showing the effects in SD rats of the anti-XX16 antibody AA07 in (A) Fab and (B) IgG format on XX16-induced cGMP levels.

FIG. 33 is a graph demonstrating the inhibition of XX16 activity by the anti-XX16 antibody AA07 IgG with an IC₅₀ of 5.96 nM using the cGMP HTRF assay. XX16 at a constant concentration of 10 nM was incubated with increasing concentrations of AA07 IgG and a dose response curve was generated as described in Example 23. The IC₅₀ value was calculated by non-linear fitting of the dose response curve using a variable slope (four parameter) fit equation.

### GENERAL DEFINITIONS

In order that the present disclosure may be more readily understood, certain terms are first defined. Additional definitions are set forth throughout the detailed description as required.

Throughout the description and claims of this specification, the words “comprise” and “contain” and variations of the words, for example “comprising” and “comprises”, mean “including but not limited to”, and do not exclude other components, integers, or steps. Moreover the singular encompasses the plural unless the context otherwise requires: in particular, where the indefinite article is used, the specification is to be understood as contemplating plurality as well as singularity, unless the context requires otherwise.

As used herein, “NPR1” and “NPR1 protein” refers to Natriuretic Peptide Receptor 1. This protein is also known as Atrial natriuretic peptide receptor type A (ANP-A, ANPR-A or NPR-A) and Guanylate cyclase A (GC-A). In some embodiments, the NPR1 referred to is human NPR1. In some embodiments the human NPR1 is has UniProt accession number P 16066 or GenBank Accession number EAW53284.1 (SEQ ID NO: 1). In some embodiments, the NPR1 referred to is mouse (Mus musculus) NPR1. In some embodiments the mouse NPR1 has NCBI Reference Sequence number NP_032753.5 (SEQ ID NO: 2). In some embodiments, the NPR1 referred to is rat (Rattus norvegicus) NPR. In some embodiments the rat NPR1 has NCBI Reference Sequence number NP_036745.1 (SEQ ID NO: 3). Exemplary NPR1 proteins are shown in Table 1. Where a constitutively active or W74R mutant is discussed herein, this mutant refers to Trp at amino acid 74 of the mature human NPR1 protein, which may also be referred to as the Trp at amino acid 106 of the hNPR1 protein shown in SEQ ID NO: 1.

TABLE 1
NPR1 Protein Sequences

| SEQ | | |
|---|---|---|
| ID NO: | Animal | Sequence (amino acid) |
| 1 | Human | MPGPRRPAGSRLRLLLLLLLPPLLLLLRGSHAGNLTVAVVLPLANTSYPWS |
| | | WARVGPAVELALAQVKARPDLLPGWTVRTVLGSSENALGVCSDTAAPLA |
| | | AVDLKWEHNPAVFLGPGCVYAAAPVGRFTAHWRVPLLTAGAPALGFGV |
| | | KDEYALTTRAGPSYAKLGDFVAALHRRLGWERQALMLYAYRPGDEEHC |
| | | FFLVEGLFMRVRDRLNITVDHLEFAEDDLSHYTRLLRTMPRKGRVIYICSS |
| | | PDAFRTLMLLALEAGLCGEDYVFFHLDIFGQSLQGGQGPAPRRPWERGDG |
| | | QDVSARQAFQAAKIITYKDPDNPEYLEFLKQLKHLAYEQFNFTMEDGLVN |
| | | TIPASFHDGLLLYIQAVTETLAHGGTVTDGENITQRMWNRSFQGVTGYLK |
| | | IDSSGDRETDFSLWDMDPENGAFRVVLNYNGTSQELVAVSGRKLNWPLG |
| | | YPPPDIPKCGFDNEDPACNQDHLSTLEVLALVGSLSLLGILIVSFFIYRKMQ |
| | | LEKELASELWRVRWEDVEPSSLERHLRSAGSRLTLSGRGSNYGSLLTTEG |
| | | QFQVFAKTAYYKGNLVAVKRVNRKRIELTRKVLFELKHMRDVQNEHLTR |
| | | FVGACTDPPNICILTEYCPRGSLQDILENESITLDWMFRYSLTNDIVKGMLF |
| | | LHNGAICSHGNLKSSNCVVDGRFVLKITDYGLESFRDLDPEQGHTVYAKK |
| | | LWTAPELLRMASPPVRGSQAGDVYSFGIILQEIALRSGVFHVEGLDLSPKEI |
| | | IERVTRGEQPPFRPSLALQSHLEELGLLMQRCWAEDPQERPPFQQIRLTLR |
| | | KFNRENSSNILDNLLSRMEQYANNLEELVEERTQAYLEEKRKAEALLYQI |
| | | LPHSVAEQLKRGETVQAEAFDSVTIYFSDIVGFTALSAESTPMQVVTLLND |
| | | LYTCFDAVIDNFDVYKVETIGDAYMVVSGLPVRNGRLHACEVARMALAL |
| | | LDAVRSFRIRHRPQEQLRLRIGIHTGPVCAGVVGLKMPRYCLFGDTVNTA |
| | | SRMESNGEALKIHLSSETKAVLEEFGGFELELRGDVEMKGKGKVRTYWL |
| | | LGERGSSTRG |
| 2 | Mouse | MPGSRRVRPRLRALLLLPPLLLLRSGHASDLTVAVVLPLTNTSYPWSWAR |
| | | VGPAVELALGRVKARPDLLPGWTVRMVLGSSENAAGVCSDTAAPLAAV |
| | | DLKWEHSPAVFLGPGCVYSAAPVGRFTAHWRVPLLTAGAPALGIGVKDE |
| | | YALTTRTGPSHVKLGDFVTALHRRLGWEHQALVLYADRLGDDRPCFFIV |
| | | EGLYMRVRERLNITVNHQEFVEGDPDHYTKLLRTVQRKGRVIYICSSPDA |
| | | FRNLMLLALDAGLTGEDYVFFHLDVFGQSLQGAQGPVPRKPWERDDGQ |
| | | DRRARQAFQAAKIITYKEPDNPEYLEFLKQLKLLADKKFNFTMEDGLKNII |
| | | PASFHDGLLLYVQAVTETLAQGGTVTDGENITQRMWNRSFQGVTGYLKI |
| | | DRNGDRDTDFSLWDMDPETGAFRVVLNFNGTSQELMAVSEHRLYWPLG |
| | | YPPPDIPKCGFDNEDPACNQDHFSTLEVLALVGSLSLVSFLIVSFFIYRKMQ |
| | | LEKELVSELWRVRWEDLQPSSLERHLRSAGSRLTLSGRGSNYGSLLTTEG |
| | | QFQVFAKTAYYKGNLVAVKRVNRKRIELTRKVLFELKHMRDVQNEHLTR |
| | | FVGACTDPPNICILTEYCPRGSLQDILENESITLDWMFRYSLTNDIVKGMLF |
| | | LHNGAIGSHGNLKSSNCVVDGRFVLKITDYGLESFRDPEPEQGHTLFAKK |
| | | LWTAPELLRMASPPARGSQAGDVYSFGIILQEIALRSGVFYVEGLDLSPKEI |
| | | IERVTRGEQPPFRPSMDLQSHLEELGQLMQRCWAEDPQERPPFQQIRLALR |
| | | KFNKENSSNILDNLLSRMEQYANNLEELVEERTQAYLEEKRKAEALLYQI |
| | | LPHSVAEQLKRGETVQAEAFDSVTIYFSDIVGFTALSAESTPMQVVTLLND |
| | | LYTCFDAVIDNFDVYKVETIGDAYMVVSGLPVRNGQLHAREVARMALAL |
| | | LDAVRSFRIRHRPQEQLRLRIGIHTGPVCAGVVGLKMPRYCLFGDTVNTA |
| | | SRMESNGEALRIHLSSETKAVLEEFDGFELELRGDVEMKGKGKVRTYWL |
| | | LGERGCSTRG |
| 3 Rat | | MPGSRRVRPRLRALLLLPPLLLLRGGHASDLTVAVVLPLTNTSYPWSWAR |
| | | VGPAVELALARVKARPDLLPGWTVRMVLGSSENAAGVCSDTAAPLAAV |
| | | DLKWEHSPAVFLGPGCVYSAAPVGRFTAHWRVPLLTAGAPALGIGVKDE |
| | | YALTTRTGPSHVKLGDFVTALHRRLGWEHQALVLYADRLGDDRPCFFIV |
| | | EGLYMRVRERLNITVNHQEFVEGDPDHYPKLLRAVRRKGRVIYICSSPDA |
| | | FRNLMLLALNAGLTGEDYVFFHLDVFGQSLKSAQGLVPQKPWERGDGQD |
| | | RSARQAFQAAKIITYKEPDNPEYLEFLKQLKLLADKKFNFTVEDGLKNIIP |
| | | ASFHDGLLLYVQAVTETLAQGGTVTDGENITQRMWNRSFQGVTGYLKID |
| | | RNGDRDTDFSLWDMDPETGAFRVVLNYNGTSQELMAVSEHKLYWPLGY |
| | | PPPDVPKCGFDNEDPACNQDHFSTLEVLALVGSLSLISFLIVSFFIYRKMQL |
| | | EKELVSELWRVRWEDLQPSSLERHLRSAGSRLTLSGRGSNYGSLLTTEGQ |
| | | FQVFAKTAYYKGNLVAVKRVNRKRIELTRKVLFELKHMRDVQNEHLTRF |
| | | VGACTDPPNICILTEYCPRGSLQDILENESITLDWMFRYSLTNDIVKGMLFL |
| | | HNGAICSHGNLKSSNCVVDGRFVLKITDYGLESFRDPEPEQGHTLFAKKL |
| | | WTAPELLRMASPPARGSQAGDVYSFGIILQEIALRSGVFYVEGLDLSPKEII |
| | | ERVTRGEQPPFRPSMDLQSHLEELGQLMQRCWAEDPQERPPFQQIRLALR |
| | | KFNKENSSNILDNLLSRMEQYANNLEELVEERTQAYLEEKRKAEALLYQI |
| | | LPHSVAEQLKRGETVQAEAFDSVTIYFSDIVGFTALSAESTPMQVVTLLND |
| | | LYTCFDAVIDNFDVYKVETIGDAYMVVSGLPVRNGQLHAREVARMALAL |
| | | LDAVRSFRIRHRPQEQLRLRIGIHTGPVCAGVVGLKMPRYCLFGDTVNTA |
| | | SRMESNGEALKIHLSSETKAVLEEFDGFELELRGDVEMKGKGKVRTYWL |
| | | LGERGCSTRG |

In various embodiments, the anti-NPR1 antibodies and antigen binding fragments disclosed herein are capable of binding to NPR1 and activating NPR1 in the absence of ANP. By virtue of this activity, the disclosed anti-NPR1 antibodies and antigen binding fragments may be useful in treating undesirable conditions, diseases and disorders including cardiovascular disorders (e.g., hypertension, peripheral vascular disease, heart failure (including but not limited to heart failure with reduced ejection fraction (HFrEF), heart failure with preserved ejection fraction (HFpEF), heart failure after acute myocardial infarct, or acute decompensated heart failure), coronary artery disease (CAD), ischemic heart disease (IHD), mitral stenosis and regurgitation, angina, hypertrophic cardiomyopathy (e.g., ventricular hypertrophy), diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI)), hypertension (e.g., resistant hypertension, hypertensive heart disease, pulmonary hypertension, pulmonary arterial hypertension, isolated systolic hypertension, resistant hypertension, or pulmonary arterial hypertension), preeclampsia, asthma, glaucoma, cytokine release syndrome, and/or a kidney disorder (e.g., diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)).

In various embodiments, the binding agents that bind to target antibodies and antigen binding fragments disclosed herein (e.g., any such binding agent described herein; e.g., AA01-AA09) are capable of binding to anti-NPR1 antibodies and antigen binding fragments thereof (e.g., any of the anti-NPR1 antibodies or antigen binding fragments thereof described herein; e.g., XX16). By virtue of this activity, the disclosed binding agents that bind to anti-NPR1 antibodies and antigen binding fragments may be useful in treating undesirable conditions, diseases and disorders associated with long acting NPR1 agonists (e.g., hypotension and any of the effects that may arise from hypotension including, but not limited to, an inadequate flow of blood to one or more body organs, stroke, heart attack, kidney failure, shock, and/or death). Additionally by virtue of this activity, the disclosed binding agents that bind to anti-NPR1 antibodies and antigen binding fragments may be useful in reversing any or all of the effects of the anti-NPR1 antibodies and antigen binding fragments such as the effects such an antibody may have on cGMP levels. In some embodiments, such a binding agent is an antibody or an antigen-binding fragment thereof, as detailed herein.

The term “antibody” as used herein refers to a whole antibody or antigen binding fragment thereof. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, and chimeric antibodies. The antibodies can be of any isotype/class (e.g., IgG, IgE, IgM, IgD, IgA and IgY) or subclass (e.g., IgG₁, IgG₂, IgG₃, IgG₄, IgA₁, and IgA₂).

The term “antigen binding fragment” refers to a fragment of an intact antibody that retains the ability to specifically bind to a given antigen (e.g., NPR1; e.g., an anti-NPR1 antibody or antigen binding fragment thereof, e.g., XX16) and/or provide a function of the intact antibody. Such fragments include Fab fragments, Fab′ fragments, monovalent fragments consisting of the VL, VH, CL and CH1 domains; F(ab′)2 fragments, bivalent fragments comprising two Fab fragments linked by a disulfide bridge at the hinge region; an Fd fragment consisting of the VH and CH1 domains; an Fv fragment consisting of the VL and VH domains, a single chain Fv fragment (scFv) consisting of the VL and VH domains connected by a linker sequence; and a single domain antibody (dAb) fragment (Ward et al., 1989 Nature 341:544-546), which consists of a VH domain or a VL domain. Such fragments include one armed antibodies.

The terms “binding agent,” “reversal agent,” “binding and/or reversal agent,” and “antidote” are used interchangeably, and, in the context of an antibody which specifically binds to NPR1 (“anti-NPR1 antibody”) or a binding agent that binds to such an anti-NPR1 antibody (or antigen binding fragment thereof), refer to a protein, polypeptide, or a complex thereof, such as an anti-idiotype antibody or a fragment thereof such as a Fab fragment. In specific aspects provided herein, the binding agent is capable of reversing (e.g., partially reversing by at least 10%, at least 20%, at least 30%, at least 40%, at least 50%, at least 60%, at least 70%) one or more hypotensive effects of an anti-NPR1 antibody (e.g., any of the antibodies described herein; e.g., antibody XX16). In specific aspects provided herein, the binding agent is capable of reversing (e.g., partially reversing by at least 10%, at least 20%, at least 30%, at least 40%, at least 50%, at least 60%, at least 70%) the effect of an anti-NPR1 antibody (e.g., any of the antibodies described herein; e.g., antibody XX16) on blood pressure. In specific aspects provided herein, the binding agent is capable of reversing (e.g., partially reversing by at least 10%, at least 20%, at least 30%, at least 40%, at least 50%, at least 60%, at least 70%) the effects of an anti-NPR1 antibody (e.g., any of the antibodies described herein; e.g., antibody XX16) on cGMP. In further specific aspects provided herein, the binding agent is capable of blocking binding of an anti-NPR1 antibody to its antigen, e.g., NPR1. As a non-limiting set of examples, the binding agent is capable of binding to one or more of the anti-NPR1 antibodies described herein. As a non-limiting set of examples, the binding agent is capable of binding to an anti-NPR1 antibody or antigen binding fragment thereof that competes with one or more of the anti-NPR1 antibodies or antigen binding fragments described herein. As a non-limiting set of examples, the binding agent is capable to of binding to an anti-NPR1 antibody or antigen binding fragment thereof that binds the same epitope as one or more of the anti-NPR1 antibodies or antigen binding fragments described herein. In a specific aspect, as used herein, the terms “anti-XX16,” “anti-XX16 antibody,” “anti-XX16 Fab,” “anti-XX16 IgG,” “XX16 binding agent,” “XX16 reversal agent,” “XX16 binding and/or reversal agent,” “XX16 antidote,” and the like, are used interchangeably and refer to a binding agent or reversal agent, such as an anti-idiotype antibody or a fragment thereof, which specifically binds to anti-NPR1 antibody XX16. Non-limiting examples of XX16 binding and/or reversal agents are described herein, for example, Table 26. In a specific aspect, the anti-NPR1 antibody or antigen binding fragment thereof may be any known antibody, e.g., may be an antibody described in WO2010/065293 or WO2020/086406; and the contents of each of these applications are incorporated herein in their entireties for this purpose.

The term “hypotension” as used herein is hypotension as defined by the medical community. A blood pressure reading lower than 90 millimeters of mercury (mm Hg) for the top number (systolic) or 60 mm Hg for the bottom number (diastolic) is generally considered low blood pressure (hypotension). In some people, low blood pressure may result in dizziness or lightheadedness, fainting, blurred or fading vision, nausea, fatigue, and/or a lack of concentration. In some cases, hypotension may present with shock, confusion, low body temperature, clammy and/or pale skin, rapid breathing, shallow breathing, weak pulse, and/or a rapid pulse. Hypotension can lead to inadequate perfusion of one or more organs (e.g., one kidney or both kidneys), and may lead to damage of the organs (e.g., permanent damage of one or more organs). In the most severe cases, hypotension can lead to death. The ANP/NPR1 system regulates blood pressure; and it is known that natriuretic peptides elicit their physiological responses through the synthesis of cyclic GMP (cGMP). See, e.g., Potter et al., Endocrine Reviews; Vol. 27, No. 1, pp 47-72 (2006); and Kamiya et al., Hear and Vessels; Vol. 35, No. 1; pp 59-68 (2019). With an increase in ANP (e.g., through agonism of NPR1, such as through use of an agonistic anti-NPR1 antibody or antigen binding fragment thereof), a subject may experience hypotension.

The terms “anti-idiotype antibody,” “anti-Id antibody,” and “anti-idiotypic antibody” are used interchangeably, and refer to an antibody or antigen binding fragments thereof (e.g., Fab fragment; e.g., dAb; e.g., one armed antibody) that specifically binds to the antigen-binding region(s) of another antibody (e.g., any known anti-NPR1 antibody or antigen binding fragment thereof; e.g., any anti-NPR1 antibody or antigen binding fragment thereof; e.g., XX16). Anti-idiotype antibodies are typically raised against the antigen-binding region(s) or complementarity determining regions (CDRs) (idiotype) of a target antibody. Anti-Idiotype antibodies can be produced by various methods described previously, see, e.g., Pan et al., 1995, FASEB J. 9:43-49. Any of the reversal and/or binding agents described herein may be also be described as an anti-idiotype antibody.

The term “single chain antibody”, “single chain Fv” or “scFv” is refers to a molecule comprising an antibody heavy chain variable domain (or region; VH) and an antibody light chain variable domain (or region; VL) connected by a linker. Such scFv molecules can have the general structures: NH2-VL-linker-VH-COOH or NH2-VH-linker-VL-COOH. Any suitable linker may be used. A non-limiting set of linkers that can be used in such single chain antibodies are described by Holliger et al. (1993), Proc. Natl. Acad. Sci. USA 90:6444-6448, Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol; the contents of each of which are herein incorporated by reference for this purpose. Such single chain antibodies are also intended to be encompassed within the term “antigen binding fragment” of an antibody. These antibody fragments are obtained using techniques known to those of skill in the art, and the fragments are screened for utility in the same manner as are intact antibodies. Without limitation, an antigen binding fragment can be produced by any suitable method known in the art. For instance, the various antigen binding fragments described herein can be produced by enzymatic or chemical modification of intact antibodies, synthesized de novo using recombinant DNA methodologies (e.g., single chain Fv), or identified using phage display libraries (see, e.g., Pini and Bracci, Curr Protein Pept Sci 2000; 1(2):155-69, the contents of which are herein incorporated by reference for this purpose). Antigen binding fragments are screened for utility (e.g., binding affinity, activity) in the same manner as are intact antibodies.

Antigen binding fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136, the contents of which are herein incorporated by reference for this purpose). Antigen binding portions of antibodies can be grafted into scaffolds based on polypeptides such as Fibronectin type III (Fn3) (see e.g., U.S. Pat. No. 6,703,199, which describes fibronectin polypeptide monobodies, the contents of which are herein incorporated by reference for this purpose).

Antigen binding fragments can be incorporated into single chain molecules comprising a pair of tandem Fv segments (VH-CH1-VH-CH1) which, together with complementary light chain polypeptides, form a pair of antigen binding regions (see Zapata et al., 1995 Protein Eng. 8(10):1057-1062; and U.S. Pat. No. 5,641,870; the contents of each of which are herein incorporated by reference for this purpose).

The term “isolated” means throughout this specification, that the immunoglobulin, antibody or polynucleotide, as the case may be, exists in a physical milieu distinct from that in which it may occur in nature. For example, a naturally-occurring polynucleotide or polypeptide present in a living organism is not isolated, but the same polynucleotide or polypeptide separated from some or all of the coexisting materials in the living organism, is isolated.

The term “isolated antibody,” as used herein, refers to an antibody that has been identified and separated from one or more (e.g., the majority) of the components (by weight) of its source environment, e.g., from the components of a hybridoma cell culture or a different cell culture that was used for its production (e.g., producer cells including but not limited to the exemplary host cells described herein that recombinantly express the antibody). The separation is performed such that it sufficiently removes components that may otherwise interfere with the suitability of the antibody for the desired applications (e.g., for therapeutic use of an anti-NPR1 antibody; e.g., for therapeutic use of any binding agent described herein including those that bind to any of the anti-NPR1 antibodies or antigen binding fragments thereof described herein). Methods for preparing isolated antibodies are known in the art and include Protein A chromatography, anion exchange chromatography, cation exchange chromatography, virus retentive filtration, and ultrafiltration.

Throughout this specification, complementarity determining regions (“CDR”) are defined according to the Kabat definition unless specified that the CDR are defined according to another definition. The precise amino acid sequence boundaries of a given CDR can be determined using any of a number of well-known schemes, including those described by Kabat et al. (1991), “Sequences of Proteins of Immunological Interest,” 5^{th} Ed. Public Health Service, National Institutes of Health, Bethesda, MD (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme); the contents of each of which are herein incorporated by reference for this purpose. For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align.

By convention, the CDR regions in the heavy chain are typically referred to as HCDR1, HCDR2 and HCDR3 and in the light chain as LCDR1, LCDR2 and LCDR3. They are numbered sequentially in the direction from the amino terminus to the carboxy terminus.

The term “antibody framework” as used herein refers to the part of the variable domain, either VL or VH, which serves as a scaffold for the antigen binding loops (CDRs) of this variable domain. In essence, it is the variable domain without the CDRs.

The terms “constant region” or “constant domain” refer to a carboxy terminal portion of the light and heavy chain which is not directly involved in binding of the antibody to antigen but exhibits various effector functions, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

The term “epitope” or “antigenic determinant” refers to a site on an antigen to which an immunoglobulin or antibody specifically binds (e.g., a specific site on the target molecule). An epitope typically includes at least 3, 4, 5, 6, 7, 8, 9, 10, 11, 12, 13, 14, or 15 consecutive or non-consecutive amino acids in a unique spatial conformation. See, e.g., Epitope Mapping Protocols in Methods in Molecular Biology, Vol. 66, G. E. Morris, Ed. (1996), the contents of which are herein incorporated by reference for this purpose. In addition, as used herein, an epitope can comprise one or more monosaccharide units of a polysaccharide to which an antibody specifically binds. In specific aspects, an epitope can be a conformational epitope. See, e.g., Thompson et al., 2009, J. of Biol. Chem. 51: 35621-35631, the contents of which are herein incorporated by reference for this purpose.

The terms “monoclonal antibody” or “monoclonal antibody composition” as used herein refers to an antibody obtained from a population of substantially homogeneous antibodies produced by a particular cell or cell line, wherein the individual antibodies comprising the population are essentially identical in sequence except for possible naturally-occurring mutations that may be present in minor amounts. A monoclonal antibody preparation displays a single binding specificity and affinity for a particular epitope. In contrast, conventional (polyclonal) antibody preparations typically include a multitude of antibodies directed against or specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be produced by a variety of techniques, including conventional methodology, e.g., the standard somatic cell hybridization technique of Kohler and Milstein (Nature 1975; 256(5517):495-7), the contents of which are herein incorporated by reference for this purpose. A monoclonal antibody may also be obtained from other suitable methods, including phage display techniques such as those described in Clackson et al. (Nature 1991; 352(6336):624-8) or Marks et al. (J Mol Biol 1991; 222(3):581-97), the contents of each of which are herein incorporated by reference for this purpose. The term “monoclonal antibody” is also not limited to antibody sequences from particular species of origin or from one single species of origin. Thus, the meaning of the term “monoclonal antibody” encompasses chimeric monoclonal antibodies such as humanized monoclonal antibodies.

The term “chimeric antibody,” as used herein, refers to antibodies in which (a) the constant region is altered, replaced, or exchanged such that the antigen binding site (variable region) is linked to a constant region of a different or altered class, effector function, and/or species; or (b) the variable region, or a portion thereof, is altered, replaced, or exchanged with a variable region, or a portion thereof, having a different or altered antigen specificity. To create a chimeric antibody, the variable region sequences from a non-human donor antibody (e.g., a mouse, rabbit, or rat donor antibody) can be linked to human constant regions using methods known in the art (see, e.g., U.S. Pat. No. 4,816,567 (Cabilly et al.), the contents of which are herein incorporated by reference for this purpose). For instance, a mouse anti-NPR1 antibody can be modified by replacing its constant region with the constant region from a human immunoglobulin. Due to the replacement with a human constant region, the chimeric antibody can retain its specificity in recognizing human NPR1 while having reduced immunogenicity in human as compared to the original mouse antibody.

As used herein, the term “humanized antibody” refers to forms of antibodies that contain at least some human sequence and at least some non-human sequence. Typically, the antibody contains human sequences and a minor portion of non-human sequences which confer binding specificity to the target antigen. Such antibodies are chimeric antibodies which contain minimal sequence derived from a non-human immunoglobulin and retain the reactivity of a non-human antibody while being less immunogenic in humans. Typically, humanized antibodies are generated by replacing hypervariable region sequences from a human acceptor antibody with hypervariable region sequences from a non-human donor antibody (e.g., a mouse, rabbit, or rat donor antibody) that binds to an antigen of interest (e.g., NPR1). In some cases, framework region sequences of the acceptor antibody may also be replaced with the corresponding sequences of the donor antibody (e.g., via affinity maturation). In addition to the sequences derived from the donor and acceptor antibodies, the humanized antibody can be further modified by the substitution of residues, either in the framework region and/or within the replaced non-human residues to refine and optimize antibody specificity, affinity, and/or activity, as discussed herein. Methods to generate humanized antibodies are known. See, e.g., Riechmann et al. (Nature 1988; 332(6162):323-7); Jones et al. (Nature 1986; 321(6069):522-5); U.S. Pat. No. 5,225,539 (Winter); and U.S. Pat. Nos. 5,530,101; 5,585,089; 5,693,762, and 6,180,370 (Queen et al.), the contents of each of which are herein incorporated by reference for this purpose.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik, et al., (2000) J Mol Biol; 296:57-86, the contents of which are herein incorporated by reference for this purpose). Human antibodies may include amino acid residues not encoded by human sequences (e.g., mutations introduced by random or site-specific mutagenesis in vitro or by somatic mutation in vivo).

The antibodies or antigen binding fragments of the disclosure may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The terms “peptide,” “polypeptide,” and “protein” are used interchangeably herein to refer to a polymer of amino acid residues. The terms encompass amino acid polymers in which one or more amino acid residues is an artificial chemical mimetic of a corresponding naturally-occurring amino acid, as well as naturally-occurring amino acid polymers and non-naturally-occurring amino acid polymers. Unless otherwise indicated, a particular polypeptide sequence also implicitly encompasses conservatively modified variants thereof.

The term “conservatively modified variant” applies to both amino acid and nucleic acid sequences. For nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence. For polypeptide sequences, conservatively modified variants include individual substitutions, deletions, or additions to a polypeptide sequence which result in the substitution of an amino acid with a chemically similar amino acid. Conservative substitution tables providing functionally similar amino acids are well known in the art. The following eight groups contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

The term “identity” or “homology” refers to a relationship between the sequences of two or more polypeptides, as determined by comparing the sequences. “Identity” also means the degree of sequence relatedness between polypeptides, as determined by the number of matches between strings of two or more amino acid residues. The percent “identity” between the two sequences is a function of the number of identical positions shared by the sequences (i.e., percent identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Additionally, or alternatively, the protein sequences of the present disclosure can further be used as a “query sequence” to perform a search against public databases to, for example, identify related sequences. For example, such searches can be performed using the BLAST program of Altschul et al. (J Mol Biol 1990; 215(3):403-10), the contents of which are herein incorporated by reference for this purpose.

Two sequences are “substantially identical” if two sequences have a specified percentage of amino acid residues or nucleotides that are the same (e.g., 60% identity, 65% identity, 70% identity, 75% identity, 80% identity, 85% identity, 90% identity, 95% identity, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or exists over a region that is 100 to 500 or 1000 or more nucleotides (or 20, 50, 200 or more amino acids) in length.

Binding “affinity” refers to the strength of interaction between antibody and antigen at single antigenic sites. Within each antigenic site, the variable region of the antibody “arm” interacts through weak non-covalent forces with antigen at numerous sites. In general, the more interactions, the stronger the affinity. Generally, such determinations can be made using a cell-based assay.

The term “Kassoc” or “Ka”, as used herein, is intended to refer to the association rate of a particular binding molecule-antigen interaction, whereas the term “Kdis” or “Kd,” as used herein, is intended to refer to the dissociation rate of a particular binding molecule-antigen interaction. The term “K_{D}”, as used herein, is intended to refer to the equilibrium dissociation constant, which is obtained from the ratio of Kd to Ka (i.e., Kd/Ka) and is expressed as a molar concentration (M). K_{D} values for antibodies can be determined using methods well established in the art. A method for determining the K_{D} of an antibody is by using surface plasmon resonance, such as a Biacore® system, or solution equilibrium titration (SET) (see Friguet et al., (1985) J. Immunol. Methods, 77(2):305-319, and Hanel et al., (2005) Anal. Biochem., 339(1):182-184), the contents of each of which are herein incorporated by reference for this purpose.

As used herein, the term “specific,” “specifically binds,” and “binds specifically” refers to a binding reaction between an antibody or antigen binding fragment (e.g., an anti-NPR1 antibody) and a target antigen (e.g., NPR1); or between a binding agent and its target antibody or antigen binding fragment thereof (e.g., an anti-NPR1 antibody or antigen binding fragment thereof, e.g., XX16) in a heterogeneous population of proteins and other biologics. Antibodies can be tested for specificity of binding by comparing binding to an appropriate antigen to binding to an irrelevant antigen or antigen mixture under a given set of conditions. If the antibody binds to the appropriate antigen with at least 2, 5, 7, and preferably 10 or more times more affinity than to the irrelevant antigen or antigen mixture, then it is considered to be specific. A “specific antibody” or a “target-specific antibody” is one that only binds the target antigen (e.g., NPR1; or e.g., an anti-NPR1 antibody or antigen binding fragment), but does not bind (or exhibits minimal binding) to other antigens. In certain embodiments, an antibody or antigen binding fragment that specifically binds the target antigen (e.g., NPR1; or e.g., an anti-NPR1 antibody or antigen binding fragment) has a K_{D} of less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In certain embodiments, the K_{D} is about 1 pM to about 600 pM. In certain embodiments, the K_{D} is between 600 pM to 1 μM, 1 μM to 100 nM, or 100 mM to 10 nM (inclusive).

In some embodiments, the anti-NPR1 antibodies or antigen binding fragments thereof described herein act as non-competitive agonists. A “non-competitive agonist” refers to a molecule which binds to an enzyme or receptor at a site distant from the binding sites of its natural ligands. The non-competitive or allosteric agonism is generally independent of the association or concentration of the natural ligands for the enzyme or receptor. Such non-competitive agonists can, for example, provide for a level of activation that can be substantially independent of natural ligands. In a specific embodiment, the anti-NPR1 antibodies or antigen binding fragments described herein (e.g., the target antibodies or antigen binding fragments thereof) are ANP non-competitive, meaning that the antibody or antigen binding fragment acts as an agonist which binds at site away from ANP binding site of NPR1 and effect agonistic activity regardless of whether or not NPR1 is bound to ANP.

In some embodiments, the anti-NPR1 antibodies or antigen binding fragments (e.g., the target antibodies or antigen binding fragments thereof) thereof act as competitive agonists. A “competitive agonist” refers to an agonist which interferes or competes with a natural ligand for its binding site on an enzyme or receptor. In a specific embodiment, the anti-NPR1 antibodies or antigen binding fragments described herein (e.g., the target antibodies or antigen binding fragments described herein) are ANP competitive, meaning that the antibody or antigen binding fragment acts as an agonist which competes with ANP at the ANP binding site of NPR1.

In some embodiments, the activation of NPR1 by an anti-NPR1 antibody or antigen binding fragment may be determined by any suitable assay. In some embodiments, the effect of a binding agent on activation of NPR1 by an anti-NPR1 antibody or antigen binding fragment may be determined by any suitable assay. An exemplary assay for determination of NPR1 activation is the production of cGMP by mammalian cells (e.g., CHO cells or a human cell line) expressing hNPR1.

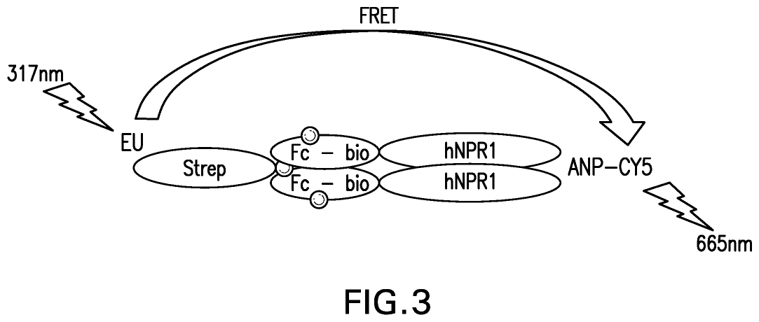

In some embodiments, the stabilization of the ANP-NPR1 complex may be determined by any suitable assay. An exemplary assay for determination of the stability of the ANP-NPR1 complex is the FRET assay described herein (see, e.g., FIG. 3).

The term “about” in relation to a numerical value×means, for example, x±10%.

Anti-NPR1 Antibodies and Antigen Binding Fragments Thereof

Below are described certain specific anti-NPR1 antibody sequences. As used herein, the term “anti-NPR1 antibody” or “antibody that binds to NPR1” refers to any form of an antibody or antigen binding fragment that specifically binds to NPR1, e.g., those binding with a K_{D} of less than 1×10⁻⁸ M, as determined by, e.g., surface plasmon resonance (SPR) spectroscopy (using Biacore™) or solution equilibrium titration (SET). The term encompasses monoclonal antibodies (including intact monoclonal antibodies), polyclonal antibodies, and biologically functional antigen binding fragments so long as they specifically bind to NPR1.

Amino acid and nucleic acid sequences of exemplary anti-NPR1 antibodies are set forth in Table 2. In some embodiments, the antibody has the heavy and light chain CDRs, VH and VL sequence, and/or the heavy and light chain sequence of any of the antibodies described in Table 2. In some embodiments, the anti-NPR1 antibody is a four-chain antibody (also referred to as an intact antibody), comprising two heavy chains and two light chains. In some embodiments, the anti-NPR1 antibody is an antigen binding fragment of an intact antibody, e.g., a functional fragment of an intact antibody selected from any of those set forth in Table 2 that retains the ability to bind NPR1 and/or provide a function of the intact antibody (e.g., activating NPR1 in the absence of ANP). In some embodiments, the anti-NPR1 antibody is an antibody having the CDRs of any heavy chain variable region and light chain variable region pair shown in Table 2. In some embodiments, the anti-NPR1 antibody is an antibody having the CDRs of any heavy and light chain pair shown in Table 2.

TABLE 2
Exemplary anti-NPR1 antibody sequences

| | | |
|---|---|---|
| WW01_LALA | | |
| SEQ ID NO: 4 | HCDR1 (Combined) | GFTFNTHYIH |
| SEQ ID NO: 5 | HCDR2 (Combined) | SISGSGSNTYYADSVKG |
| SEQ ID NO: 6 | HCDR3 (Combined) | ERGYVYYHMFDP |
| SEQ ID NO: 7 | HCDR1 (Kabat) | THYIH |
| SEQ ID NO: 5 | HCDR2 (Kabat) | SISGSGSNTYYADSVKG |
| SEQ ID NO: 6 | HCDR3 (Kabat) | ERGYVYYHMFDP |
| SEQ ID NO: 8 | HCDR1 (Chothia) | GFTFNTH |
| SEQ ID NO: 9 | HCDR2 (Chothia) | SGSGSN |
| SEQ ID NO: 6 | HCDR3 (Chothia) | ERGYVYYHMFDP |
| SEQ ID NO: 10 | HCDR1 (IMGT) | GFTFNTHY |
| SEQ ID NO: 11 | HCDR2 (IMGT) | ISGSGSNT |
| SEQ ID NO: 12 | HCDR3 (IMGT) | ARERGYVYYHMFDP |
| SEQ ID NO: 13 | VH | QVQLLESGGGLVQPGGSLRLSCAASGFTFNTHYIHWVRQAPGKGLEWVSSI |
| | | SGSGSNTYYADSVKGRFTISRDNSKNTLYLQMNSLRAEDTAVYYCARERG |
| | | YVYYHMFDPWGQGTLVTVSS |
| SEQ ID NO: 14 | DNA VH | caggtgcaattgctggaaagcggcggtggcctggtgcagccgggggcagcctgcgtctgagctgcgcggcgtccg |
| | | gattcacctttaacactcattacatccattgggtgcgccaggcccccggcaaaggtctcgagtgggtttcctctatctctgg |
| | | ttctggttctaacacctactatgcggatagcgtgaaaggccgctttaccatcagccgcgataattcgaaaaacaccctgta |
| | | tctgcaaatgaacagcctgcgtgcggaagatacggccgtgtattattgcgcgcgtgaacgtggttacgtttactaccatat |
| | | gttcgatccgtggggccaaggcaccctggtgactgttagctca |
| SEQ ID NO: 15 | Heavy Chain | QVQLLESGGGLVQPGGSLRLSCAASGFTFNTHYIHWVRQAPGKGLEWVSSI |
| | | SGSGSNTYYADSVKGRFTISRDNSKNTLYLQMNSLRAEDTAVYYCARERG |
| | | YVYYHMFDPWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKRVEPKSCDKTHTCPPCPAPEAAGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 16 | DNA Heavy Chain | caggtgcaattgctggaaagcggcggtggcctggtgcagccgggtggcagcctgcgtctgagctgcgcggcgtccg |
| | | gattcacctttaacactcattacatccattgggtgcgccaggcccccggcaaaggtctcgagtgggtttcctctatctctgg |
| | | ttctggttctaacacctactatgcggatagcgtgaaaggccgctttaccatcagccgcgataattcgaaaaacaccctgta |
| | | tctgcaaatgaacagcctgcgtgcggaagatacggccgtgtattattgcgcgcgtgaacgtggttacgtttactaccatat |
| | | gttcgatccgtggggccaaggcaccctggtgactgttagctcagcctccaccaagggtccatcggtcttccccctggca |
| | | ccctcctccaagagcacctctgggggcacagcggccctgggctgcctggtcaaggactacttccccgaaccggtgac |
| | | ggtgtcgtggaactcaggcgccctgaccagcggcgtgcacaccttcccggctgtcctacagtcctcaggactctactcc |
| | | ctcagcagcgtggtgaccgtgccctccagcagcttgggcacccagacctacatctgcaacgtgaatcacaagcccag |
| | | caacaccaaggtggacaagagagttgagcccaaatcttgtgacaaaactcacacatgcccaccgtgcccagcacctg |
| | | aagcagcggggggaccgtcagtcttcctcttccccccaaaacccaaggacaccctcatgatctcccggacccctgagg |
| | | tcacatgcgtggtggtggacgtgagccacgaagaccctgaggtcaagttcaactggtacgtggacggcgtggaggtg |
| | | cataatgccaagacaaagccgcgggaggagcagtacaacagcacgtaccgggtggtcagcgtcctcaccgtcctgc |
| | | accaggactggctgaatggcaaggagtacaagtgcaaggtctccaacaaagccctcccagcccccatcgagaaaac |
| | | catctccaaagccaaagggcagccccgagaaccacaggtgtacaccctgcccccatcccgggaggagatgaccaag |
| | | aaccaggtcagcctgacctgcctggtcaaaggcttctatcccagcgacatcgccgtggagtgggagagcaatgggca |
| | | gccggagaacaactacaagaccacgcctcccgtgctggactccgacggctccttcttcctctacagcaagctcaccgt |
| | | ggacaagagcaggtggcagcaggggaacgtcttctcatgctccgtgatgcatgaggctctgcacaaccactacacgc |
| | | agaagagcctctccctgtctccgggtaaa |
| SEQ ID NO: 17 | LCDR1 (Combined) | RASQSITRNYLA |
| SEQ ID NO: 18 | LCDR2 (Combined) | GASSRAT |
| SEQ ID NO: 19 | LCDR3 (Combined) | QQHSMYPRT |
| SEQ ID NO: 17 | LCDR1 (Kabat) | RASQSITRNYLA |
| SEQ ID NO: 18 | LCDR2 (Kabat) | GASSRAT |
| SEQ ID NO: 19 | LCDR3 (Kabat) | QQHSMYPRT |
| SEQ ID NO: 20 | LCDR1 (Chothia) | SQSITRNY |
| SEQ ID NO: 21 | LCDR2 (Chothia) | GAS |
| SEQ ID NO: 22 | LCDR3 (Chothia) | HSMYPR |
| SEQ ID NO: 23 | LCDR1 (IMGT) | QSITRNY |
| SEQ ID NO: 21 | LCDR2 (IMGT) | GAS |
| SEQ ID NO: 19 | LCDR3 (IMGT) | QQHSMYPRT |
| SEQ ID NO: 24 | VL | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIK |
| SEQ ID NO: 25 | DNA VL | gatatcgtgctgacccagagcccggcgaccctgagcctgagcccgggtgaacgtgccaccctgagctgcagagcga |
| | | gccagtctatcactcgtaactacctggcttggtaccagcagaaaccgggccaggccccgcgtctattaatctacggtgct |
| | | tcttctcgtgcgaccggcattccggcgcgttttagcggcagcggatccggcaccgatttcaccctgaccattagcagcct |
| | | ggaaccggaagactttgcggtgtattattgccagcagcattctatgtacccgcgtacctttggccagggcacgaaagttg |
| | | aaattaaa |
| SEQ ID NO: 26 | Light Chain | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| SEQ ID NO: 27 | DNA Light Chain | gatatcgtgctgacccagagcccggcgaccctgagcctgagcccgggtgaacgtgccaccctgagctgcagagcga |
| | | gccagtctatcactcgtaactacctggcttggtaccagcagaaaccgggccaggccccgcgtctattaatctacggtgct |
| | | tcttctcgtgcgaccggcattccggcgcgttttagcggcagcggatccggcaccgatttcaccctgaccattagcagcct |
| | | ggaaccggaagactttgcggtgtattattgccagcagcattctatgtacccgcgtacctttggccagggcacgaaagttg |
| | | aaattaaacgtacggtggccgctcccagcgtgttcatcttcccccccagcgacgagcagctgaagagcggcaccgcc |
| | | agcgtggtgtgcctgctgaacaacttctacccccgggaggccaaggtgcagtggaaggtggacaacgccctgcaga |
| | | gcggcaacagccaggaaagcgtcaccgagcaggacagcaaggactccacctacagcctgagcagcaccctgaccc |
| | | tgagcaaggccgactacgagaagcacaaggtgtacgcctgcgaggtgacccaccagggcctgtccagccccgtgac |
| | | caagagcttcaaccggggcgagtgt |
| WW03_LALA | | |
| SEQ ID NO: 28 | HCDR1 (Combined) | GFTFSSYWMN |
| SEQ ID NO: 29 | HCDR2 (Combined) | AISSDGSYTYYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 29 | HCDR2 (Kabat) | AISSDGSYTYYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 33 | HCDR2 (Chothia) | SSDGSY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 35 | HCDR2 (IMGT) | ISSDGSYT |
| SEQ ID NO: 36 | HCDR3 (IMGT) | ARDRYSMIYSYGAGAFDY |
| SEQ ID NO: 37 | VH | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSS |
| SEQ ID NO: 38 | DNA VH | caggtgcaattgctggaaagcggcggtggcctggtgcagccgggtggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctca |
| SEQ ID NO: 39 | Heavy Chain | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 40 | DNA Heavy Chain | caggtgcaattgctggaaagcggcggtggcctggtgcagccgggtggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctcagcctccaccaagggtccatcggt |
| | | cttccccctggcaccctcctccaagagcacctctgggggcacagcggccctgggctgcctggtcaaggactacttccc |
| | | cgaaccggtgacggtgtcgtggaactcaggcgccctgaccagcggcgtgcacaccttcccggctgtcctacagtcctc |
| | | aggactctactccctcagcagcgtggtgaccgtgccctccagcagcttgggcacccagacctacatctgcaacgtgaat |
| | | cacaagcccagcaacaccaaggtggacaagagagttgagcccaaatcttgtgacaaaactcacacatgcccaccgtg |
| | | cccagcacctgaagcagcggggggaccgtcagtcttcctcttccccccaaaacccaaggacaccctcatgatctcccg |
| | | gacccctgaggtcacatgcgtggtggtggacgtgagccacgaagaccctgaggtcaagttcaactggtacgtggacg |
| | | gcgtggaggtgcataatgccaagacaaagccgcgggaggagcagtacaacagcacgtaccgggtggtcagcgtcct |
| | | caccgtcctgcaccaggactggctgaatggcaaggagtacaagtgcaaggtctccaacaaagccctcccagccccca |
| | | tcgagaaaaccatctccaaagccaaagggcagccccgagaaccacaggtgtacaccctgcccccatcccgggagga |
| | | gatgaccaagaaccaggtcagcctgacctgcctggtcaaaggcttctatcccagcgacatcgccgtggagtgggaga |
| | | gcaatgggcagccggagaacaactacaagaccacgcctcccgtgctggactccgacggctccttcttcctctacagca |
| | | agctcaccgtggacaagagcaggtggcagcaggggaacgtcttctcatgctccgtgatgcatgaggctctgcacaacc |
| | | actacacgcagaagagcctctccctgtctccgggtaaa |
| SEQ ID NO: 41 | LCDR1 (Combined) | RASQGISSYLA |
| SEQ ID NO: 42 | LCDR2 (Combined) | TASTLQS |
| SEQ ID NO: 43 | LCDR3 (Combined) | MQSYEKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 43 | LCDR3 (Kabat) | MQSYEKPRT |
| SEQ ID NO: 44 | LCDR1 (Chothia) | SQGISSY |
| SEQ ID NO: 45 | LCDR2 (Chothia) | TAS |
| SEQ ID NO: 46 | LCDR3 (Chothia) | SYEKPR |
| SEQ ID NO: 47 | LCDR1 (IMGT) | QGISSY |
| SEQ ID NO: 45 | LCDR2 (IMGT) | TAS |
| SEQ ID NO: 43 | LCDR3 (IMGT) | MQSYEKPRT |
| SEQ ID NO: 48 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCMQSYEKPRTFGQGTKV |
| | | EIK |
| SEQ ID NO: 49 | DNA VL | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgcatgcagtcttacgaaaaaccgcgtacctttggccagggcacgaaagttgaaa |
| | | ttaaa |
| SEQ ID NO: 50 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCMQSYEKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 51 | DNA Light Chain | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgcatgcagtcttacgaaaaaccgcgtacctttggccagggcacgaaagttgaaa |
| | | ttaaacgtacggtggccgctcccagcgtgttcatcttcccccccagcgacgagcagctgaagagcggcaccgccagc |
| | | gtggtgtgcctgctgaacaacttctacccccgggaggccaaggtgcagtggaaggtggacaacgccctgcagagcg |
| | | gcaacagccaggaaagcgtcaccgagcaggacagcaaggactccacctacagcctgagcagcaccctgaccctga |
| | | gcaaggccgactacgagaagcacaaggtgtacgcctgcgaggtgacccaccagggcctgtccagccccgtgacca |
| | | agagcttcaaccggggcgagtgt |
| WW05_LALA | | |
| SEQ ID NO: 52 | HCDR1 (Combined) | GYSFSNYWIG |
| SEQ ID NO: 53 | HCDR2 (Combined) | IIYPDVSYTRYSPSFQG |
| SEQ ID NO: 54 | HCDR3 (Combined) | YWSEAYTFDY |
| SEQ ID NO: 55 | HCDR1 (Kabat) | NYWIG |
| SEQ ID NO: 53 | HCDR2 (Kabat) | IIYPDVSYTRYSPSFQG |
| SEQ ID NO: 54 | HCDR3 (Kabat) | YWSEAYTFDY |
| SEQ ID NO: 56 | HCDR1 (Chothia) | GYSFSNY |
| SEQ ID NO: 57 | HCDR2 (Chothia) | YPDVSY |
| SEQ ID NO: 54 | HCDR3 (Chothia) | YWSEAYTFDY |
| SEQ ID NO: 58 | HCDR1 (IMGT) | GYSFSNYW |
| SEQ ID NO: 59 | HCDR2 (IMGT) | IYPDVSYT |
| SEQ ID NO: 60 | HCDR3 (IMGT) | ARYWSEAYTFDY |
| SEQ ID NO: 61 | VH | QVQLVQSGAEVKKPGESLKISCKGSGYSFSNYWIGWVRQMPGKGLEWMG |
| | | IIYPDVSYTRYSPSFQGQVTISADKSISTAYLQWSSLKASDTAMYYCARYWS |
| | | EAYTFDYWGQGTLVTVSS |
| SEQ ID NO: 62 | DNA VH | caggtgcaattggtgcagagcggtgcggaagtgaaaaaaccgggcgaaagcctgaaaattagctgcaaaggctccg |
| | | gatatagcttctctaactactggatcggttgggtgcgccagatgccgggcaaaggtctcgagtggatgggcatcatctac |
| | | ccggacgttagctacacccgttatagcccgagctttcagggccaggtgaccattagcgcggataaaagcatcagcacc |
| | | gcgtatctgcaatggagcagcctgaaagcgagcgataccgcgatgtattattgcgcgcgttactggtctgaagcttacac |
| | | tttcgattactggggccaaggcaccctggtgactgttagctca |
| SEQ ID NO: 63 | Heavy Chain | QVQLVQSGAEVKKPGESLKISCKGSGYSFSNYWIGWVRQMPGKGLEWMG |
| | | IIYPDVSYTRYSPSFQGQVTISADKSISTAYLQWSSLKASDTAMYYCARYWS |
| | | EAYTFDYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICNV |
| | | NHKPSNTKVDKRVEPKSCDKTHTCPPCPAPEAAGGPSVFLFPPKPKDTLMIS |
| | | RTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFF |
| | | LYSKLTVDKSRWQQGNVFSCSVMHEALHNHYTQKSLSLSPGK |
| SEQ ID NO: 64 | DNA Heavy Chain | caggtgcaattggtgcagagcggtgcggaagtgaaaaaaccgggcgaaagcctgaaaattagctgcaaaggctccg |
| | | gatatagcttctctaactactggatcggttgggtgcgccagatgccgggcaaaggtctcgagtggatgggcatcatctac |
| | | ccggacgttagctacacccgttatagcccgagctttcagggccaggtgaccattagcgcggataaaagcatcagcacc |
| | | gcgtatctgcaatggagcagcctgaaagcgagcgataccgcgatgtattattgcgcgcgttactggtctgaagcttacac |
| | | tttcgattactggggccaaggcaccctggtgactgttagctcagcctccaccaagggtccatcggtcttccccctggcac |
| | | cctcctccaagagcacctctgggggcacagcggccctgggctgcctggtcaaggactacttccccgaaccggtgacg |
| | | gtgtcgtggaactcaggcgccctgaccagcggcgtgcacaccttcccggctgtcctacagtcctcaggactctactccc |
| | | tcagcagcgtggtgaccgtgccctccagcagcttgggcacccagacctacatctgcaacgtgaatcacaagcccagc |
| | | aacaccaaggtggacaagagagttgagcccaaatcttgtgacaaaactcacacatgcccaccgtgcccagcacctga |
| | | agcagcggggggaccgtcagtcttcctcttccccccaaaacccaaggacaccctcatgatctcccggacccctgaggt |
| | | cacatgcgtggtggtggacgtgagccacgaagaccctgaggtcaagttcaactggtacgtggacggcgtggaggtgc |
| | | ataatgccaagacaaagccgcgggaggagcagtacaacagcacgtaccgggtggtcagcgtcctcaccgtcctgca |
| | | ccaggactggctgaatggcaaggagtacaagtgcaaggtctccaacaaagccctcccagcccccatcgagaaaacc |
| | | atctccaaagccaaagggcagccccgagaaccacaggtgtacaccctgcccccatcccgggaggagatgaccaaga |
| | | accaggtcagcctgacctgcctggtcaaaggcttctatcccagcgacatcgccgtggagtgggagagcaatgggcag |
| | | ccggagaacaactacaagaccacgcctcccgtgctggactccgacggctccttcttcctctacagcaagctcaccgtg |
| | | gacaagagcaggtggcagcaggggaacgtcttctcatgctccgtgatgcatgaggctctgcacaaccactacacgca |
| | | gaagagcctctccctgtctccgggtaaa |
| SEQ ID NO: 65 | LCDR1 (Combined) | SGDNIRKKYVF |
| SEQ ID NO: 66 | LCDR2 (Combined) | GDNDRPS |
| SEQ ID NO: 67 | LCDR3 (Combined) | GTYTLLFTSKV |
| SEQ ID NO: 65 | LCDR1 (Kabat) | SGDNIRKKYVF |
| SEQ ID NO: 66 | LCDR2 (Kabat) | GDNDRPS |
| SEQ ID NO: 67 | LCDR3 (Kabat) | GTYTLLFTSKV |
| SEQ ID NO: 68 | LCDR1 (Chothia) | DNIRKKY |
| SEQ ID NO: 69 | LCDR2 (Chothia) | GDN |
| SEQ ID NO: 70 | LCDR3 (Chothia) | YTLLFTSK |
| SEQ ID NO: 71 | LCDR1 (IMGT) | NIRKKY |
| SEQ ID NO: 69 | LCDR2 (IMGT) | GDN |
| SEQ ID NO: 67 | LCDR3 (IMGT) | GTYTLLFTSKV |
| SEQ ID NO: 72 | VL | DIELTQPPSVSVSPGQTASITCSGDNIRKKYVFWYQQKPGQAPVLVIYGDND |
| | | RPSGIPERFSGSNSGNTATLTISGTQAEDEADYYCGTYTLLFTSKVFGGGTK |
| | | LTVL |
| SEQ ID NO: 73 | DNA VL | gatatcgaactgacccagccgccgagcgtgagcgtgagtccgggccagaccgcgagcattacctgtagcggcgata |
| | | acatccgtaaaaaatacgttttctggtaccagcagaaaccgggccaggcgccggtgctggtgatctacggtgacaacg |
| | | accgtccgagcggcatcccggaacgttttagcggatccaacagcggcaacaccgcgaccctgaccattagcggcacc |
| | | caggcggaagacgaagcggattattactgcggtacttacactctgctgttcacttctaaagtgtttggcggggcacgaa |
| | | gttaaccgtccta |
| SEQ ID NO: 74 | Light Chain | DIELTQPPSVSVSPGQTASITCSGDNIRKKYVFWYQQKPGQAPVLVIYGDND |
| | | RPSGIPERFSGSNSGNTATLTISGTQAEDEADYYCGTYTLLFTSKVFGGGTK |
| | | LTVLGQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTVEK |
| | | TVAPTECS |
| SEQ ID NO: 75 | DNA Light Chain | gatatcgaactgacccagccgccgagcgtgagcgtgagtccggccagaccgcgagcattacctgtagcggcgata |
| | | acatccgtaaaaaatacgttttctggtaccagcagaaaccgggccaggcgccggtgctggtgatctacggtgacaacg |
| | | accgtccgagcggcatcccggaacgttttagcggatccaacagcggcaacaccgcgaccctgaccattagcggcacc |
| | | caggcggaagacgaagcggattattactgcggtacttacactctgctgttcacttctaaagtgtttggcggcggcacgaa |
| | | gttaaccgtcctaggtcagcccaaggctgccccctcggtcactctgttcccgccctcctctgaggagcttcaagccaaca |
| | | aggccacactggtgtgtctcataagtgacttctacccgggagccgtgacagtggcctggaaggcagatagcagcccc |
| | | gtcaaggcgggagtggagaccaccacaccctccaaacaaagcaacaacaagtacgcggccagcagctatctgagcc |
| | | tgacgcctgagcagtggaagtcccacagaagctacagctgccaggtcacgcatgaagggagcaccgtggagaagac |
| | | agtggcccctacagaatgttca |
| WW06_LALA | | |
| SEQ ID NO: 76 | HCDR1 (Combined) | GYSFTSYWIA |
| SEQ ID NO: 77 | HCDR2 (Combined) | RIDPDNSYTRYSPSFQG |
| SEQ ID NO: 78 | HCDR3 (Combined) | WLSPGYALGEQPAGMDH |
| SEQ ID NO: 79 | HCDR1 (Kabat) | SYWIA |
| SEQ ID NO: 77 | HCDR2 (Kabat) | RIDPDNSYTRYSPSFQG |
| SEQ ID NO: 78 | HCDR3 (Kabat) | WLSPGYALGEQPAGMDH |
| SEQ ID NO: 80 | HCDR1 (Chothia) | GYSFTSY |
| SEQ ID NO: 81 | HCDR2 (Chothia) | DPDNSY |
| SEQ ID NO: 78 | HCDR3 (Chothia) | WLSPGYALGEQPAGMDH |
| SEQ ID NO: 82 | HCDR1 (IMGT) | GYSFTSYW |
| SEQ ID NO: 83 | HCDR2 (IMGT) | IDPDNSYT |
| SEQ ID NO: 84 | HCDR3 (IMGT) | ARWLSPGYALGEQPAGMDH |
| SEQ ID NO: 85 | VH | QVQLVQSGAEVKKPGESLKISCKGSGYSFTSYWIAWVRQMPGKGLEWMG |
| | | RIDPDNSYTRYSPSFQGQVTISADKSISTAYLQWSSLKASDTAMYYCARWL |
| | | SPGYALGEQPAGMDHWGQGTLVTVSS |
| SEQ ID NO: 86 | DNA VH | caggtgcaattggtgcagagcggtgcggaagtgaaaaaaccgggcgaaagcctgaaaattagctgcaaaggctccg |
| | | gatatagcttcacttcttactggatcgcttgggtgcgccagatgccgggcaaaggtctcgagtggatgggccgtatcgac |
| | | ccggacaacagctacacccgttatagcccgagctttcagggccaggtgaccattagcgcggataaaagcatcagcac |
| | | cgcgtatctgcaatggagcagcctgaaagcgagcgataccgcgatgtattattgcgcgcgttggctgtctccgggttac |
| | | gctctgggtgaacagccggctggtatggatcattggggccaaggcaccctggtgactgttagctca |
| SEQ ID NO: 87 | Heavy Chain | QVQLVQSGAEVKKPGESLKISCKGSGYSFTSYWIAWVRQMPGKGLEWMG |
| | | RIDPDNSYTRYSPSFQGQVTISADKSISTAYLQWSSLKASDTAMYYCARWL |
| | | SPGYALGEQPAGMDHWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 88 | DNA Heavy Chain | caggtgcaattggtgcagagcggtgcggaagtgaaaaaaccgggcgaaagcctgaaaattagctgcaaaggctccg |
| | | gatatagcttcacttcttactggatcgcttgggtgcgccagatgccgggcaaaggtctcgagtggatgggccgtatcgac |
| | | ccggacaacagctacacccgttatagcccgagctttcagggccaggtgaccattagcgcggataaaagcatcagcac |
| | | cgcgtatctgcaatggagcagcctgaaagcgagcgataccgcgatgtattattgcgcgcgttggctgtctccgggttac |
| | | gctctgggtgaacagccggctggtatggatcattggggccaaggcaccctggtgactgttagctcagcctccaccaag |
| | | ggtccatcggtcttccccctggcaccctcctccaagagcacctctgggggcacagcggccctgggctgcctggtcaag |
| | | gactacttccccgaaccggtgacggtgtcgtggaactcaggcgccctgaccagcggcgtgcacaccttcccggctgtc |
| | | ctacagtcctcaggactctactccctcagcagcgtggtgaccgtgccctccagcagcttgggcacccagacctacatct |
| | | gcaacgtgaatcacaagcccagcaacaccaaggtggacaagagagttgagcccaaatcttgtgacaaaactcacaca |
| | | tgcccaccgtgcccagcacctgaagcagcggggggaccgtcagtcttcctcttccccccaaaacccaaggacaccct |
| | | catgatctcccggacccctgaggtcacatgcgtggtggtggacgtgagccacgaagaccctgaggtcaagttcaactg |
| | | gtacgtggacggcgtggaggtgcataatgccaagacaaagccgcgggaggagcagtacaacagcacgtaccgggt |
| | | ggtcagcgtcctcaccgtcctgcaccaggactggctgaatggcaaggagtacaagtgcaaggtctccaacaaagccct |
| | | cccagcccccatcgagaaaaccatctccaaagccaaagggcagccccgagaaccacaggtgtacaccctgccccca |
| | | tcccgggaggagatgaccaagaaccaggtcagcctgacctgcctggtcaaaggcttctatcccagcgacatcgccgt |
| | | ggagtgggagagcaatgggcagccggagaacaactacaagaccacgcctcccgtgctggactccgacggctccttc |
| | | ttcctctacagcaagctcaccgtggacaagagcaggtggcagcaggggaacgtcttctcatgctccgtgatgcatgag |
| | | gctctgcacaaccactacacgcagaagagcctctccctgtctccgggtaaa |
| SEQ ID NO: 89 | LCDR1 (Combined) | TGSSSNIGAGYAVH |
| SEQ ID NO: 90 | LCDR2 (Combined) | SNNKRPS |
| SEQ ID NO: 91 | LCDR3 (Combined) | QSYDLQKSSRV |
| SEQ ID NO: 89 | LCDR1 (Kabat) | TGSSSNIGAGYAVH |
| SEQ ID NO: 90 | LCDR2 (Kabat) | SNNKRPS |
| SEQ ID NO: 91 | LCDR3 (Kabat) | QSYDLQKSSRV |
| SEQ ID NO: 92 | LCDR1 (Chothia) | SSSNIGAGYA |
| SEQ ID NO: 93 | LCDR2 (Chothia) | SNN |
| SEQ ID NO: 94 | LCDR3 (Chothia) | YDLQKSSR |
| SEQ ID NO: 95 | LCDR1 (IMGT) | SSNIGAGYA |
| SEQ ID NO: 93 | LCDR2 (IMGT) | SNN |
| SEQ ID NO: 91 | LCDR3 (IMGT) | QSYDLQKSSRV |
| SEQ ID NO: 96 | VL | DIVLTQPPSVSGAPGQRVTISCTGSSSNIGAGYAVHWYQQLPGTAPKLLIYS |
| | | NNKRPSGVPDRFSGSKSGTSASLAITGLQAEDEADYYCQSYDLQKSSRVFG |
| | | GGTKLTVL |
| SEQ ID NO: 97 | DNA VL | gatatcgtgctgacccagccgccgagcgtgagcggtgcaccgggccagcgcgtgaccattagctgtaccggcagca |
| | | gcagcaacattggtgctggttacgctgtgcattggtaccagcagctgccgggcacggcgccgaaactgctgatctactc |
| | | taacaacaaacgcccgagcggcgtgccggatcgctttagcggatccaaaagcggcaccagcgccagcctggcgatt |
| | | accggcctgcaagcagaagacgaagcggattattactgccagtcttacgacctgcagaaatcttctcgtgtgtttggcgg |
| | | cggcacgaagttaaccgtccta |
| SEQ ID NO: 98 | Light Chain | DIVLTQPPSVSGAPGQRVTISCTGSSSNIGAGYAVHWYQQLPGTAPKLLIYS |
| | | NNKRPSGVPDRFSGSKSGTSASLAITGLQAEDEADYYCQSYDLQKSSRVFG |
| | | GGTKLTVLGQPKAAPSVTLFPPSSEELQANKATLVCLISDFYPGAVTVAWK |
| | | ADSSPVKAGVETTTPSKQSNNKYAASSYLSLTPEQWKSHRSYSCQVTHEGS |
| | | TVEKTVAPTECS |
| SEQ ID NO: 99 | DNA Light Chain | gatatcgtgctgacccagccgccgagcgtgagcggtgcaccgggccagcgcgtgaccattagctgtaccggcagca |
| | | gcagcaacattggtgctggttacgctgtgcattggtaccagcagctgccgggcacggcgccgaaactgctgatctactc |
| | | taacaacaaacgcccgagcggcgtgccggatcgctttagcggatccaaaagcggcaccagcgccagcctggcgatt |
| | | accggcctgcaagcagaagacgaagcggattattactgccagtcttacgacctgcagaaatcttctcgtgtgtttggcgg |
| | | cggcacgaagttaaccgtcctaggtcagcccaaggctgccccctcggtcactctgttcccgccctcctctgaggagctt |
| | | caagccaacaaggccacactggtgtgtctcataagtgacttctacccgggagccgtgacagtggcctggaaggcagat |
| | | agcagccccgtcaaggcgggagtggagaccaccacaccctccaaacaaagcaacaacaagtacgcggccagcag |
| | | ctatctgagcctgacgcctgagcagtggaagtcccacagaagctacagctgccaggtcacgcatgaagggagcaccg |
| | | tggagaagacagtggcccctacagaatgttca |
| XX01 LALA | | |
| SEQ ID NO: 4 | HCDR1 (Combined) | GFTFNTHYIH |
| SEQ ID NO: 100 | HCDR2 (Combined) | SISSSGQSTYYADSVKG |
| SEQ ID NO: 6 | HCDR3 (Combined) | ERGYVYYHMFDP |
| SEQ ID NO: 7 | HCDR1 (Kabat) | THYIH |
| SEQ ID NO: 100 | HCDR2 (Kabat) | SISSSGQSTYYADSVKG |
| SEQ ID NO: 6 | HCDR3 (Kabat) | ERGYVYYHMFDP |
| SEQ ID NO: 8 | HCDR1 (Chothia) | GFTFNTH |
| SEQ ID NO: 101 | HCDR2 (Chothia) | SSSGQS |
| SEQ ID NO: 6 | HCDR3 (Chothia) | ERGYVYYHMFDP |
| SEQ ID NO: 10 | HCDR1 (IMGT) | GFTFNTHY |
| SEQ ID NO: 102 | HCDR2 (IMGT) | ISSSGQST |
| SEQ ID NO: 12 | HCDR3 (IMGT) | ARERGYVYYHMFDP |
| SEQ ID NO: 103 | VH | EVQLLESGGGLVQPGGSLRLSCAASGFTFNTHYIHWVRQAPGKGLEWVSSI |
| | | SSSGQSTYYADSVKGRFTISRDNSKNTLYLQMNSLRAEDTAVYYCARERG |
| | | YVYYHMFDPWGQGTLVTVSS |
| SEQ ID NO: 104 | DNA VH | gaggtgcaattgctggaaagcggcggtggcctggtgcagccgggtggcagcctgcgtctgagctgcgcggcgtccg |
| | | gattcacctttaacactcattacatccattgggtgcgccaggcccccggcaaaggtctcgagtgggtttcctctatctcttct |
| | | tctggccagtctacttactatgcggatagcgtgaaaggccgctttaccatcagccgcgataattcgaaaaacaccctgtat |
| | | ctgcaaatgaacagcctgcgtgcggaagatacggccgtgtattattgcgcgcgtgaacgtggttacgtttactaccatat |
| | | gttcgatccgtggggccaaggcaccctggtgactgttagctca |
| SEQ ID NO: 105 | Heavy Chain | EVQLLESGGGLVQPGGSLRLSCAASGFTFNTHYIHWVRQAPGKGLEWVSSI |
| | | SSSGQSTYYADSVKGRFTISRDNSKNTLYLQMNSLRAEDTAVYYCARERG |
| | | YVYYHMFDPWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKRVEPKSCDKTHTCPPCPAPEAAGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 106 | DNA Heavy Chain | gaggtgcaattgctggaaagcggcggtggcctggtgcagccgggtggcagcctgcgtctgagctgcgcggcgtccg |
| | | gattcacctttaacactcattacatccattgggtgcgccaggcccccggcaaaggtctcgagtgggtttcctctatctcttct |
| | | tctggccagtctacttactatgcggatagcgtgaaaggccgctttaccatcagccgcgataattcgaaaaacaccctgtat |
| | | ctgcaaatgaacagcctgcgtgcggaagatacggccgtgtattattgcgcgcgtgaacgtggttacgtttactaccatat |
| | | gttcgatccgtggggccaaggcaccctggtgactgttagctcagcctccaccaagggtccatcggtcttccccctggca |
| | | ccctcctccaagagcacctctgggggcacagcggccctgggctgcctggtcaaggactacttccccgaaccggtgac |
| | | ggtgtcgtggaactcaggcgccctgaccagcggcgtgcacaccttcccggctgtcctacagtcctcaggactctactcc |
| | | ctcagcagcgtggtgaccgtgccctccagcagcttgggcacccagacctacatctgcaacgtgaatcacaagcccag |
| | | caacaccaaggtggacaagagagttgagcccaaatcttgtgacaaaactcacacatgcccaccgtgcccagcacctg |
| | | aagcagcggggggaccgtcagtcttcctcttccccccaaaacccaaggacaccctcatgatctcccggacccctgagg |
| | | tcacatgcgtggtggtggacgtgagccacgaagaccctgaggtcaagttcaactggtacgtggacggcgtggaggtg |
| | | cataatgccaagacaaagccgcgggaggagcagtacaacagcacgtaccgggtggtcagcgtcctcaccgtcctgc |
| | | accaggactggctgaatggcaaggagtacaagtgcaaggtctccaacaaagccctcccagcccccatcgagaaaac |
| | | catctccaaagccaaagggcagccccgagaaccacaggtgtacaccctgcccccatcccgggaggagatgaccaag |
| | | aaccaggtcagcctgacctgcctggtcaaaggcttctatcccagcgacatcgccgtggagtgggagagcaatgggca |
| | | gccggagaacaactacaagaccacgcctcccgtgctggactccgacggctccttcttcctctacagcaagctcaccgt |
| | | ggacaagagcaggtggcagcaggggaacgtcttctcatgctccgtgatgcatgaggctctgcacaaccactacacgc |
| | | agaagagcctctccctgtctccgggtaaa |
| SEQ ID NO: 17 | LCDR1 (Combined) | RASQSITRNYLA |
| SEQ ID NO: 18 | LCDR2 (Combined) | GASSRAT |
| SEQ ID NO: 19 | LCDR3 (Combined) | QQHSMYPRT |
| SEQ ID NO: 17 | LCDR1 (Kabat) | RASQSITRNYLA |
| SEQ ID NO: 18 | LCDR2 (Kabat) | GASSRAT |
| SEQ ID NO: 19 | LCDR3 (Kabat) | QQHSMYPRT |
| SEQ ID NO: 20 | LCDR1 (Chothia) | SQSITRNY |
| SEQ ID NO: 21 | LCDR2 (Chothia) | GAS |
| SEQ ID NO: 22 | LCDR3 (Chothia) | HSMYPR |
| SEQ ID NO: 23 | LCDR1 (IMGT) | QSITRNY |
| SEQ ID NO: 21 | LCDR2 (IMGT) | GAS |
| SEQ ID NO: 19 | LCDR3 (IMGT) | QQHSMYPRT |
| SEQ ID NO: 24 | VL | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIK |
| SEQ ID NO: 25 | DNA VL | gatatcgtgctgacccagagcccggcgaccctgagcctgagcccgggtgaacgtgccaccctgagctgcagagcga |
| | | gccagtctatcactcgtaactacctggcttggtaccagcagaaaccgggccaggccccgcgtctattaatctacggtgct |
| | | tcttctcgtgcgaccggcattccggcgcgttttagcggcagcggatccggcaccgatttcaccctgaccattagcagcct |
| | | ggaaccggaagactttgcggtgtattattgccagcagcattctatgtacccgcgtacctttggccagggcacgaaagttg |
| | | aaattaaa |
| SEQ ID NO: 26 | Light Chain | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| SEQ ID NO: 27 | DNA Light Chain | gatatcgtgctgacccagagcccggcgaccctgagcctgagcccgggtgaacgtgccaccctgagctgcagagcga |
| | | gccagtctatcactcgtaactacctggcttggtaccagcagaaaccgggccaggccccgcgtctattaatctacggtgct |
| | | tcttctcgtgcgaccggcattccggcgcgttttagcggcagcggatccggcaccgatttcaccctgaccattagcagcct |
| | | ggaaccggaagactttgcggtgtattattgccagcagcattctatgtacccgcgtacctttggccagggcacgaaagttg |
| | | aaattaaacgtacggtggccgctcccagcgtgttcatcttcccccccagcgacgagcagctgaagagcggcaccgcc |
| | | agcgtggtgtgcctgctgaacaacttctacccccgggaggccaaggtgcagtggaaggtggacaacgccctgcaga |
| | | gcggcaacagccaggaaagcgtcaccgagcaggacagcaaggactccacctacagcctgagcagcaccctgaccc |
| | | tgagcaaggccgactacgagaagcacaaggtgtacgcctgcgaggtgacccaccagggcctgtccagccccgtgac |
| | | caagagcttcaaccggggcgagtgt |
| XX01_DAPA | | |
| SEQ ID NO: 4 | HCDR1 (Combined) | GFTFNTHYIH |
| SEQ ID NO: 100 | HCDR2 (Combined) | SISSSGQSTYYADSVKG |
| SEQ ID NO: 6 | HCDR3 (Combined) | ERGYVYYHMFDP |
| SEQ ID NO: 7 | HCDR1 (Kabat) | THYIH |
| SEQ ID NO: 100 | HCDR2 (Kabat) | SISSSGQSTYYADSVKG |
| SEQ ID NO: 6 | HCDR3 (Kabat) | ERGYVYYHMFDP |
| SEQ ID NO: 8 | HCDR1 (Chothia) | GFTFNTH |
| SEQ ID NO: 101 | HCDR2 (Chothia) | SSSGQS |
| SEQ ID NO: 6 | HCDR3 (Chothia) | ERGYVYYHMFDP |
| SEQ ID NO: 10 | HCDR1 (IMGT) | GFTFNTHY |
| SEQ ID NO: 102 | HCDR2 (IMGT) | ISSSGQST |
| SEQ ID NO: 12 | HCDR3 (IMGT) | ARERGYVYYHMFDP |
| SEQ ID NO: 103 | VH | EVQLLESGGGLVQPGGSLRLSCAASGFTFNTHYIHWVRQAPGKGLEWVSSI |
| | | SSSGQSTYYADSVKGRFTISRDNSKNTLYLQMNSLRAEDTAVYYCARERG |
| | | YVYYHMFDPWGQGTLVTVSS |
| SEQ ID NO: 107 | DNA VH | gaagtgcagctgcttgagtccgggggtggactggtgcagcccggaggatccctgcgcctgagctgcgctgcatccgg |
| | | cttcaccttcaacacgcactacatccattgggtcagacaggccccaggaaaaggcctggaatgggtgtcctccatctcct |
| | | cgtcggggcagtcaacctactacgcggactccgtcaagggccggtttaccattagccgggacaacagcaagaatacc |
| | | ctgtacctccaaatgaactcgctgagggccgaagataccgccgtgtattactgtgcccgcgagagaggctacgtgtact |
| | | accacatgttcgacccgtggggacagggtactctcgtgactgtgtcttct |
| SEQ ID NO: 108 | Heavy Chain | EVQLLESGGGLVQPGGSLRLSCAASGFTFNTHYIHWVRQAPGKGLEWVSSI |
| | | SSSGQSTYYADSVKGRFTISRDNSKNTLYLQMNSLRAEDTAVYYCARERG |
| | | YVYYHMFDPWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKRVEPKSCDKTHTCPPCPAPELLGGPSVFLFPPKPKDT |
| | | LMISRTPEVTCVVVAVSHEDPEVKFNWYVDGVEVHNAKTKPREEQYNSTY |
| | | RVVSVLTVLHQDWLNGKEYKCKVSNKALAAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 109 | DNA Heavy Chain | gaagtgcagctgcttgagtccgggggtggactggtgcagcccggaggatccctgcgcctgagctgcgctgcatccgg |
| | | cttcaccttcaacacgcactacatccattgggtcagacaggccccaggaaaaggcctggaatgggtgtcctccatctcct |
| | | cgtcggggcagtcaacctactacgcggactccgtcaagggccggtttaccattagccgggacaacagcaagaatacc |
| | | ctgtacctccaaatgaactcgctgagggccgaagataccgccgtgtattactgtgcccgcgagagaggctacgtgtact |
| | | accacatgttcgacccgtggggacagggtactctcgtgactgtgtcttctgcgagcactaagggcccgtcagtgttccc |
| | | gctggctccatcgtcgaagtccacctccggaggaaccgcagcactcggttgcctggtcaaggactacttccctgagcc |
| | | agtgaccgtgtcgtggaacagcggagccctgacttccggcgtgcacacttttcccgcggtgctgcagtcctccggtctg |
| | | tactccctttcgtccgtggtcaccgtgccgtcgtctagcctgggcacccagacctacatctgcaacgtgaaccacaagcc |
| | | gtccaacaccaaagtggataagcgggtggagccgaagtcctgcgataagacacacacgtgcccgccatgtccagcg |
| | | cctgaattgcttggcggaccttccgtgttcctgttcccgcctaagcccaaggacaccttgatgattagccggactcccga |
| | | agtcacctgtgtggtggtggcagtgtcccacgaggaccccgaggtcaagtttaattggtacgtggacggcgtcgaagt |
| | | gcacaacgccaagactaagccccgggaggaacagtacaacagcacctaccgggtcgtgtccgtgctgaccgtgctg |
| | | caccaggactggctgaatgggaaagagtacaagtgcaaagtgtccaacaaggccttggccgctcctatcgaaaaaact |
| | | atcagcaaggctaagggacagccgagggaaccccaagtctacaccctgcccccttcacgcgaagagatgaccaaga |
| | | atcaagtgtcgctgacctgcctcgtcaagggattctacccctccgacattgcggtggagtgggagtccaacggccagc |
| | | ccgagaacaactacaagactactccgcccgtgctggactccgacggcagcttcttcctgtattccaagctgaccgtgga |
| | | caagtcccggtggcagcaaggaaacgtgttctcctgctcggtcatgcacgaagccctgcacaaccactatacgcagaa |
| | | gtccctgtccttgagcccggggaaa |
| SEQ ID NO: 17 | LCDR1 (Combined) | RASQSITRNYLA |
| SEQ ID NO: 18 | LCDR2 (Combined) | GASSRAT |
| SEQ ID NO: 19 | LCDR3 (Combined) | QQHSMYPRT |
| SEQ ID NO: 17 | LCDR1 (Kabat) | RASQSITRNYLA |
| SEQ ID NO: 18 | LCDR2 (Kabat) | GASSRAT |
| SEQ ID NO: 19 | LCDR3 (Kabat) | QQHSMYPRT |
| SEQ ID NO: 20 | LCDR1 (Chothia) | SQSITRNY |
| SEQ ID NO: 21 | LCDR2 (Chothia) | GAS |
| SEQ ID NO: 22 | LCDR3 (Chothia) | HSMYPR |
| SEQ ID NO: 23 | LCDR1 (IMGT) | QSITRNY |
| SEQ ID NO: 21 | LCDR2 (IMGT) | GAS |
| SEQ ID NO: 19 | LCDR3 (IMGT) | QQHSMYPRT |
| SEQ ID NO: 24 | VL | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIK |
| SEQ ID NO: 110 | DNA VL | gacatcgtgctgactcagtcccctgcgactctgagcctgtcaccgggagaacgggccaccctctcttgccgcgcctcc |
| | | caatccattactcggaactacctggcctggtatcagcagaagccaggacaggcccctaggcttctgatctacggggcc |
| | | agctcaagagcaactggcatcccggctcgcttctccggttcgggaagcggcaccgacttcaccctgacaatttcgtccc |
| | | tcgaacccgaggatttcgccgtgtactactgccaacagcactccatgtacccccggacctttgggcagggaaccaaagt |
| | | cgagatcaag |
| SEQ ID NO: 26 | Light Chain | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| SEQ ID NO: 111 | DNA Light Chain | gacatcgtgctgactcagtcccctgcgactctgagcctgtcaccgggagaacgggccaccctctcttgccgcgcctcc |
| | | caatccattactcggaactacctggcctggtatcagcagaagccaggacaggcccctaggcttctgatctacggggcc |
| | | agctcaagagcaactggcatcccggctcgcttctccggttcgggaagcggcaccgacttcaccctgacaatttcgtccc |
| | | tcgaacccgaggatttcgccgtgtactactgccaacagcactccatgtacccccggacctttgggcagggaaccaaagt |
| | | cgagatcaagcgtacggtggccgctcccagcgtgttcatcttcccccccagcgacgagcagctgaagagcggcaccg |
| | | ccagcgtggtgtgcctgctgaacaacttctacccccgggaggccaaggtgcagtggaaggtggacaacgccctgcag |
| | | agcggcaacagccaggagagcgtcaccgagcaggacagcaaggactccacctacagcctgagcagcaccctgacc |
| | | ctgagcaaggccgactacgagaagcataaggtgtacgcctgcgaggtgacccaccagggcctgtccagccccgtga |
| | | ccaagagcttcaacaggggcgagtgc |
| XX01_N30S_DAPA | | |
| SEQ ID NO: 112 | HCDR1 (Combined) | GFTFSTHYIH |
| SEQ ID NO: 100 | HCDR2 (Combined) | SISSSGQSTYYADSVKG |
| SEQ ID NO: 6 | HCDR3 (Combined) | ERGYVYYHMFDP |
| SEQ ID NO: 7 | HCDR1 (Kabat) | THYIH |
| SEQ ID NO: 100 | HCDR2 (Kabat) | SISSSGQSTYYADSVKG |
| SEQ ID NO: 6 | HCDR3 (Kabat) | ERGYVYYHMFDP |
| SEQ ID NO: 113 | HCDR1 (Chothia) | GFTFSTH |
| SEQ ID NO: 101 | HCDR2 (Chothia) | SSSGQS |
| SEQ ID NO: 6 | HCDR3 (Chothia) | ERGYVYYHMFDP |
| SEQ ID NO: 114 | HCDR1 (IMGT) | GFTFSTHY |
| SEQ ID NO: 102 | HCDR2 (IMGT) | ISSSGQST |
| SEQ ID NO: 12 | HCDR3 (IMGT) | ARERGYVYYHMFDP |
| SEQ ID NO: 115 | VH | EVQLLESGGGLVQPGGSLRLSCAASGFTFSTHYIHWVRQAPGKGLEWVSSI |
| | | SSSGQSTYYADSVKGRFTISRDNSKNTLYLQMNSLRAEDTAVYYCARERG |
| | | YVYYHMFDPWGQGTLVTVSS |
| SEQ ID NO: 116 | DNA VH | gaagtgcagctgcttgagtccgggggtggactggtgcagcccggaggatccctgcgcctgagctgcgctgcatccgg |
| | | cttcaccttcagcacgcactacatccattgggtcagacaggccccaggaaaaggcctggaatgggtgtcctccatctcc |
| | | tcgtcggggcagtcaacctactacgcggactccgtcaagggccggtttaccattagccgggacaacagcaagaatacc |
| | | ctgtacctccaaatgaactcgctgagggccgaagataccgccgtgtattactgtgcccgcgagagaggctacgtgtact |
| | | accacatgttcgacccgtggggacagggtactctcgtgactgtgtcttct |
| SEQ ID NO: 117 | Heavy Chain | EVQLLESGGGLVQPGGSLRLSCAASGFTFSTHYIHWVRQAPGKGLEWVSSI |
| | | SSSGQSTYYADSVKGRFTISRDNSKNTLYLQMNSLRAEDTAVYYCARERG |
| | | YVYYHMFDPWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKRVEPKSCDKTHTCPPCPAPELLGGPSVFLFPPKPKDT |
| | | LMISRTPEVTCVVVAVSHEDPEVKFNWYVDGVEVHNAKTKPREEQYNSTY |
| | | RVVSVLTVLHQDWLNGKEYKCKVSNKALAAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 118 | DNA Heavy Chain | gaagtgcagctgcttgagtccgggggtggactggtgcagcccggaggatccctgcgcctgagctgcgctgcatccgg |
| | | cttcaccttcagcacgcactacatccattgggtcagacaggccccaggaaaaggcctggaatgggtgtcctccatctcc |
| | | tcgtcggggcagtcaacctactacgcggactccgtcaagggccggtttaccattagccgggacaacagcaagaatacc |
| | | ctgtacctccaaatgaactcgctgagggccgaagataccgccgtgtattactgtgcccgcgagagaggctacgtgtact |
| | | accacatgttcgacccgtggggacagggtactctcgtgactgtgtcttctgcgagcactaagggcccgtcagtgttccc |
| | | gctggctccatcgtcgaagtccacctccggaggaaccgcagcactcggttgcctggtcaaggactacttccctgagcc |
| | | agtgaccgtgtcgtggaacagcggagccctgacttccggcgtgcacacttttcccgcggtgctgcagtcctccggtctg |
| | | tactccctttcgtccgtggtcaccgtgccgtgtctagcctgggcacccagacctacatctgcaacgtgaaccacaagcc |
| | | gtccaacaccaaagtggataagcgggtggagccgaagtcctgcgataagacacacacgtgcccgccatgtccagcg |
| | | cctgaattgcttggcggaccttccgtgttcctgttcccgcctaagcccaaggacaccttgatgattagccggactcccga |
| | | agtcacctgtgtggtggtggcagtgtcccacgaggaccccgaggtcaagtttaattggtacgtggacggcgtcgaagt |
| | | gcacaacgccaagactaagccccgggaggaacagtacaacagcacctaccgggtcgtgtccgtgctgaccgtgctg |
| | | caccaggactggctgaatgggaaagagtacaagtgcaaagtgtccaacaaggccttggccgctcctatcgaaaaaact |
| | | atcagcaaggctaagggacagccgagggaaccccaagtctacaccctgcccccttcacgcgaagagatgaccaaga |
| | | atcaagtgtcgctgacctgcctcgtcaagggattctacccctccgacattgcggtggagtgggagtccaacggccagc |
| | | ccgagaacaactacaagactactccgcccgtgctggactccgacggcagcttcttcctgtattccaagctgaccgtgga |
| | | caagtcccggtggcagcaaggaaacgtgttctcctgctcggtcatgcacgaagccctgcacaaccactatacgcagaa |
| | | gtccctgtccttgagcccggggaaa |
| SEQ ID NO: 17 | LCDR1 (Combined) | RASQSITRNYLA |
| SEQ ID NO: 18 | LCDR2 (Combined) | GASSRAT |
| SEQ ID NO: 19 | LCDR3 (Combined) | QQHSMYPRT |
| SEQ ID NO: 17 | LCDR1 (Kabat) | RASQSITRNYLA |
| SEQ ID NO: 18 | LCDR2 (Kabat) | GASSRAT |
| SEQ ID NO: 19 | LCDR3 (Kabat) | QQHSMYPRT |
| SEQ ID NO: 20 | LCDR1 (Chothia) | SQSITRNY |
| SEQ ID NO: 21 | LCDR2 (Chothia) | GAS |
| SEQ ID NO: 22 | LCDR3 (Chothia) | HSMYPR |
| SEQ ID NO: 23 | LCDR1 (IMGT) | QSITRNY |
| SEQ ID NO: 21 | LCDR2 (IMGT) | GAS |
| SEQ ID NO: 19 | LCDR3 (IMGT) | QQHSMYPRT |
| SEQ ID NO: 24 | VL | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIK |
| SEQ ID NO: 110 | DNA VL | gacatcgtgctgactcagtcccctgcgactctgagcctgtcaccgggagaacgggccaccctctcttgccgcgcctcc |
| | | caatccattactcggaactacctggcctggtatcagcagaagccaggacaggcccctaggcttctgatctacggggcc |
| | | agctcaagagcaactggcatcccggctcgcttctccggttcgggaagcggcaccgacttcaccctgacaatttcgtccc |
| | | tcgaacccgaggatttcgccgtgtactactgccaacagcactccatgtacccccggacctttgggcagggaaccaaagt |
| | | cgagatcaag |
| SEQ ID NO: 26 | Light Chain | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| SEQ ID NO: 111 | DNA Light Chain | gacatcgtgctgactcagtcccctgcgactctgagcctgtcaccgggagaacgggccaccctctcttgccgcgcctcc |
| | | caatccattactcggaactacctggcctggtatcagcagaagccaggacaggcccctaggcttctgatctacggggcc |
| | | agctcaagagcaactggcatcccggctcgcttctccggttcgggaagcggcaccgacttcaccctgacaatttcgtccc |
| | | tcgaacccgaggatttcgccgtgtactactgccaacagcactccatgtacccccggacctttgggcagggaaccaaagt |
| | | cgagatcaagcgtacggtggccgctcccagcgtgttcatcttcccccccagcgacgagcagctgaagagcggcaccg |
| | | ccagcgtggtgtgcctgctgaacaacttctacccccgggaggccaaggtgcagtggaaggtggacaacgccctgcag |
| | | agcggcaacagccaggagagcgtcaccgagcaggacagcaaggactccacctacagcctgagcagcaccctgacc |
| | | ctgagcaaggccgactacgagaagcataaggtgtacgcctgcgaggtgacccaccagggcctgtccagccccgtga |
| | | ccaagagcttcaacaggggcgagtgc |
| XX03_LALA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 122 | VH | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 123 | DNA VH | gaggtgcaattgctggaaagcggcggtggcctggtgcagccgggggcagcctgcgtctgagctgcgcggcgtccg |
| | | gattcaccttttcttcttactggatgaactgggtgcgccaggccccgggcaaaggtctcgagtgggtttccgttatcgaat |
| | | ctaaaggcaactacatcttctatgcggatagcgtgaaaggccgctttaccatcagccgcgataattcgaaaaacaccctg |
| | | tatctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctca |
| SEQ ID NO: 124 | Heavy Chain | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 125 | DNA Heavy Chain | gaggtgcaattgctggaaagcggcggtggcctggtgcagccgggtggcagcctgcgtctgagctgcgcggcgtccg |
| | | gattcaccttttcttcttactggatgaactgggtgcgccaggccccgggcaaaggtctcgagtgggtttccgttatcgaat |
| | | ctaaaggcaactacatcttctatgcggatagcgtgaaaggccgctttaccatcagccgcgataattcgaaaaacaccctg |
| | | tatctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctcagcctccaccaagggtccatcggt |
| | | cttccccctggcaccctcctccaagagcacctctgggggcacagcggccctgggctgcctggtcaaggactacttccc |
| | | cgaaccggtgacggtgtcgtggaactcaggcgccctgaccagcggcgtgcacaccttcccggctgtcctacagtcctc |
| | | aggactctactccctcagcagcgtggtgaccgtgccctccagcagcttgggcacccagacctacatctgcaacgtgaat |
| | | cacaagcccagcaacaccaaggtggacaagagagttgagcccaaatcttgtgacaaaactcacacatgcccaccgtg |
| | | cccagcacctgaagcagcggggggaccgtcagtcttcctcttccccccaaaacccaaggacaccctcatgatctcccg |
| | | gacccctgaggtcacatgcgtggtggtggacgtgagccacgaagaccctgaggtcaagttcaactggtacgtggacg |
| | | gcgtggaggtgcataatgccaagacaaagccgcgggaggagcagtacaacagcacgtaccgggtggtcagcgtcct |
| | | caccgtcctgcaccaggactggctgaatggcaaggagtacaagtgcaaggtctccaacaaagccctcccagccccca |
| | | tcgagaaaaccatctccaaagccaaagggcagccccgagaaccacaggtgtacaccctgcccccatcccgggagga |
| | | gatgaccaagaaccaggtcagcctgacctgcctggtcaaaggcttctatcccagcgacatcgccgtggagtgggaga |
| | | gcaatgggcagccggagaacaactacaagaccacgcctcccgtgctggactccgacggctccttcttcctctacagca |
| | | agctcaccgtggacaagagcaggtggcagcaggggaacgtcttctcatgctccgtgatgcatgaggctctgcacaacc |
| | | actacacgcagaagagcctctccctgtctccgggtaaa |
| SEQ ID NO: 41 | LCDR1 (Combined) | RASQGISSYLA |
| SEQ ID NO: 42 | LCDR2 (Combined) | TASTLQS |
| SEQ ID NO: 43 | LCDR3 (Combined) | MQSYEKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 43 | LCDR3 (Kabat) | MQSYEKPRT |
| SEQ ID NO: 44 | LCDR1 (Chothia) | SQGISSY |
| SEQ ID NO: 45 | LCDR2 (Chothia) | TAS |
| SEQ ID NO: 46 | LCDR3 (Chothia) | SYEKPR |
| SEQ ID NO: 47 | LCDR1 (IMGT) | QGISSY |
| SEQ ID NO: 45 | LCDR2 (IMGT) | TAS |
| SEQ ID NO: 43 | LCDR3 (IMGT) | MQSYEKPRT |
| SEQ ID NO: 48 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCMQSYEKPRTFGQGTKV |
| | | EIK |
| SEQ ID NO: 49 | DNA VL | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgcatgcagtcttacgaaaaaccgcgtacctttggccagggcacgaaagttgaaa |
| | | ttaaa |
| SEQ ID NO: 50 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCMQSYEKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 51 | DNA Light Chain | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgcatgcagtcttacgaaaaaccgcgtacctttggccagggcacgaaagttgaaa |
| | | ttaaacgtacggtggccgctcccagcgtgttcatcttcccccccagcgacgagcagctgaagagcggcaccgccagc |
| | | gtggtgtgcctgctgaacaacttctacccccgggaggccaaggtgcagtggaaggtggacaacgccctgcagagcg |
| | | gcaacagccaggaaagcgtcaccgagcaggacagcaaggactccacctacagcctgagcagcaccctgaccctga |
| | | gcaaggccgactacgagaagcacaaggtgtacgcctgcgaggtgacccaccagggcctgtccagccccgtgacca |
| | | agagcttcaaccggggcgagtgt |
| XX04_LALA | | |
| SEQ ID NO: 28 | HCDR1 (Combined) | GFTFSSYWMN |
| SEQ ID NO: 29 | HCDR2 (Combined) | AISSDGSYTYYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 29 | HCDR2 (Kabat) | AISSDGSYTYYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 33 | HCDR2 (Chothia) | SSDGSY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 35 | HCDR2 (IMGT) | ISSDGSYT |
| SEQ ID NO: 36 | HCDR3 (IMGT) | ARDRYSMIYSYGAGAFDY |
| SEQ ID NO: 37 | VH | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSS |
| SEQ ID NO: 38 | DNA VH | caggtgcaattgctggaaagcggcggtggcctggtgcagccgggtggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctca |
| SEQ ID NO: 39 | Heavy Chain | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 40 | DNA Heavy Chain | caggtgcaattgctggaaagcggcggtggcctggtgcagccgggtggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctcagcctccaccaagggtccatcggt |
| | | cttccccctggcaccctcctccaagagcacctctgggggcacagcggccctgggctgcctggtcaaggactacttccc |
| | | cgaaccggtgacggtgtcgtggaactcaggcgccctgaccagcggcgtgcacaccttcccggctgtcctacagtcctc |
| | | aggactctactccctcagcagcgtggtgaccgtgccctccagcagcttgggcacccagacctacatctgcaacgtgaat |
| | | cacaagcccagcaacaccaaggtggacaagagagttgagcccaaatcttgtgacaaaactcacacatgcccaccgtg |
| | | cccagcacctgaagcagcggggggaccgtcagtcttcctcttccccccaaaacccaaggacaccctcatgatctcccg |
| | | gacccctgaggtcacatgcgtggtggtggacgtgagccacgaagaccctgaggtcaagttcaactggtacgtggacg |
| | | gcgtggaggtgcataatgccaagacaaagccgcgggaggagcagtacaacagcacgtaccgggtggtcagcgtcct |
| | | caccgtcctgcaccaggactggctgaatggcaaggagtacaagtgcaaggtctccaacaaagccctcccagccccca |
| | | tcgagaaaaccatctccaaagccaaagggcagccccgagaaccacaggtgtacaccctgcccccatcccgggagga |
| | | gatgaccaagaaccaggtcagcctgacctgcctggtcaaaggcttctatcccagcgacatcgccgtggagtgggaga |
| | | gcaatgggcagccggagaacaactacaagaccacgcctcccgtgctggactccgacggctccttcttcctctacagca |
| | | agctcaccgtggacaagagcaggtggcagcaggggaacgtcttctcatgctccgtgatgcatgaggctctgcacaacc |
| | | actacacgcagaagagcctctccctgtctccgggtaaa |
| SEQ ID NO: 41 | LCDR1 (Combined) | RASQGISSYLA |
| SEQ ID NO: 42 | LCDR2 (Combined) | TASTLQS |
| SEQ ID NO: 126 | LCDR3 (Combined) | QQEWVKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 126 | LCDR3 (Kabat) | QQEWVKPRT |
| SEQ ID NO: 44 | LCDR1 (Chothia) | SQGISSY |
| SEQ ID NO: 45 | LCDR2 (Chothia) | TAS |
| SEQ ID NO: 127 | LCDR3 (Chothia) | EWVKPR |
| SEQ ID NO: 47 | LCDR1 (IMGT) | QGISSY |
| SEQ ID NO: 45 | LCDR2 (IMGT) | TAS |
| SEQ ID NO: 126 | LCDR3 (IMGT) | QQEWVKPRT |
| SEQ ID NO: 128 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWVKPRTFGQGTKV |
| | | EIK |
| SEQ ID NO: 129 | DNA VL | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcaggaatgggttaaaccgcgtacctttggccagggcacgaaagttgaa |
| | | attaaa |
| SEQ ID NO: 130 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWVKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 131 | DNA Light Chain | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcaggaatgggttaaaccgcgtacctttggccagggcacgaaagttgaa |
| | | attaaacgtacggtggccgctcccagcgtgttcatcttcccccccagcgacgagcagctgaagagcggcaccgccag |
| | | cgtggtgtgcctgctgaacaacttctacccccgggaggccaaggtgcagtggaaggtggacaacgccctgcagagc |
| | | ggcaacagccaggaaagcgtcaccgagcaggacagcaaggactccacctacagcctgagcagcaccctgaccctg |
| | | agcaaggccgactacgagaagcacaaggtgtacgcctgcgaggtgacccaccagggcctgtccagccccgtgacc |
| | | aagagcttcaaccggggcgagtgt |
| XX06_LALA | | |
| SEQ ID NO: 28 | HCDR1 (Combined) | GFTFSSYWMN |
| SEQ ID NO: 29 | HCDR2 (Combined) | AISSDGSYTYYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 29 | HCDR2 (Kabat) | AISSDGSYTYYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 33 | HCDR2 (Chothia) | SSDGSY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 35 | HCDR2 (IMGT) | ISSDGSYT |
| SEQ ID NO: 36 | HCDR3 (IMGT) | ARDRYSMIYSYGAGAFDY |
| SEQ ID NO: 37 | VH | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSS |
| SEQ ID NO: 132 | DNA VH | caggtgcaattgctggaaagcggcggtggcctggtgcagccgggtggcagcctgcgtctgagctgcgcggcgtccg |
| | | gattcaccttttcttcttactggatgaactgggtgcgccaggccccaggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctca |
| SEQ ID NO: 39 | Heavy Chain | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 133 | DNA Heavy Chain | caggtgcaattgctggaaagcggcggtggcctggtgcagccgggtggcagcctgcgtctgagctgcgcggcgtccg |
| | | gattcaccttttcttcttactggatgaactgggtgcgccaggccccaggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctcagcctccaccaagggtccatcggt |
| | | cttccccctggcaccctcctccaagagcacctctgggggcacagcggccctgggctgcctggtcaaggactacttccc |
| | | cgaaccggtgacggtgtcgtggaactcaggcgccctgaccagcggcgtgcacaccttcccggctgtcctacagtcctc |
| | | aggactctactccctcagcagcgtggtgaccgtgccctccagcagcttgggcacccagacctacatctgcaacgtgaat |
| | | cacaagcccagcaacaccaaggtggacaagagagttgagcccaaatcttgtgacaaaactcacacatgcccaccgtg |
| | | cccagcacctgaagcagcggggggaccgtcagtcttcctcttccccccaaaacccaaggacaccctcatgatctcccg |
| | | gacccctgaggtcacatgcgtggtggtggacgtgagccacgaagaccctgaggtcaagttcaactggtacgtggacg |
| | | gcgtggaggtgcataatgccaagacaaagccgcgggaggagcagtacaacagcacgtaccgggtggtcagcgtcct |
| | | caccgtcctgcaccaggactggctgaatggcaaggagtacaagtgcaaggtctccaacaaagccctcccagccccca |
| | | tcgagaaaaccatctccaaagccaaagggcagccccgagaaccacaggtgtacaccctgcccccatcccgggagga |
| | | gatgaccaagaaccaggtcagcctgacctgcctggtcaaaggcttctatcccagcgacatcgccgtggagtgggaga |
| | | gaatgggcagccggagaacaactacaagaccacgcctcccgtgctggactccgacggctccttcttcctctacagca |
| | | agctcaccgtggacaagagcaggtggcagcaggggaacgtcttctcatgctccgtgatgcatgaggctctgcacaacc |
| | | actacacgcagaagagcctctccctgtctccgggtaaa |
| SEQ ID NO: 41 | LCDR1 (Combined) | RASQGISSYLA |
| SEQ ID NO: 42 | LCDR2 (Combined) | TASTLQS |
| SEQ ID NO: 134 | LCDR3 (Combined) | QQTWRKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 134 | LCDR3 (Kabat) | QQTWRKPRT |
| SEQ ID NO: 44 | LCDR1 (Chothia) | SQGISSY |
| SEQ ID NO: 45 | LCDR2 (Chothia) | TAS |
| SEQ ID NO: 135 | LCDR3 (Chothia) | TWRKPR |
| SEQ ID NO: 47 | LCDR1 (IMGT) | QGISSY |
| SEQ ID NO: 45 | LCDR2 (IMGT) | TAS |
| SEQ ID NO: 134 | LCDR3 (IMGT) | QQTWRKPRT |
| SEQ ID NO: 136 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIK |
| SEQ ID NO: 137 | DNA VL | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcagacttggcgtaaaccgcgtacctttggccagggcacgaaagttgaa |
| | | attaaa |
| SEQ ID NO: 138 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 139 | DNA Light Chain | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcagacttggcgtaaaccgcgtacctttggccagggcacgaaagttgaa |
| | | attaaacgtacggtggccgctcccagcgtgttcatcttcccccccagcgacgagcagctgaagagcggcaccgccag |
| | | cgtggtgtgcctgctgaacaacttctacccccgggaggccaaggtgcagtggaaggtggacaacgccctgcagagc |
| | | ggcaacagccaggaaagcgtcaccgagcaggacagcaaggactccacctacagcctgagcagcaccctgaccctg |
| | | agcaaggccgactacgagaagcacaaggtgtacgcctgcgaggtgacccaccagggcctgtccagccccgtgacc |
| | | aagagcttcaaccggggcgagtgt |
| XX06_DAPA | | |
| SEQ ID NO: 28 | HCDR1 (Combined) | GFTFSSYWMN |
| SEQ ID NO: 29 | HCDR2 (Combined) | AISSDGSYTYYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 29 | HCDR2 (Kabat) | AISSDGSYTYYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 33 | HCDR2 (Chothia) | SSDGSY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 35 | HCDR2 (IMGT) | ISSDGSYT |
| SEQ ID NO: 36 | HCDR3 (IMGT) | ARDRYSMIYSYGAGAFDY |
| SEQ ID NO: 37 | VH | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSS |
| SEQ ID NO: 140 | DNA VH | caagtgcagctgcttgagagcggtggcggactggtgcagccagggggatccttgcgcctgtcatgcgctgcgtcggg |
| | | gttcaccttctcgtcctactggatgaactgggtcagacagggctccggggaagaatgggtgtccgccatttcct |
| | | ccgacggctcctacacttactacgccgatagcgtcaagggccggttcaccatctcccgggacaattcgaagaacaccc |
| | | tgtacctccaaatgaactcactgcgcgccgaggacactgcggtgtattactgtgcccgggataggtacagcatgatcta |
| | | ctcctacggtgccggagcctttgactactggggacagggaacccttgtgaccgtgtctagc |
| SEQ ID NO: 141 | Heavy Chain | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPELLGGPSVFLFPP |
| | | KPKDTLMISRTPEVTCVVVAVSHEDPEVKFNWYVDGVEVHNAKTKPREEQ |
| | | YNSTYRVVSVLTVLHQDWLNGKEYKCKVSNKALAAPIEKTISKAKGQPRE |
| | | PQVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSPG |
| | | K |
| SEQ ID NO: 142 | DNA Heavy Chain | caagtgcagctgcttgagagcggtggcggactggtgcagccagggggatccttgcgcctgtcatgcgctgcgtcggg |
| | | gttcaccttctcgtcctactggatgaactgggtcagacaggctccggggaagggactcgaatgggtgtccgccatttcct |
| | | ccgacggctcctacacttactacgccgatagcgtcaagggccggttcaccatctcccgggacaattcgaagaacaccc |
| | | tgtacctccaaatgaactcactgcgcgccgaggacactgcggtgtattactgtgcccgggataggtacagcatgatcta |
| | | ctcctacggtgccggagcctttgactactggggacagggaacccttgtgaccgtgtctagcgcgtccactaagggccc |
| | | gtcagtgttcccgctggctccatcgtcgaagtccacctccggaggaaccgcagcactcggttgcctggtcaaggactac |
| | | ttccctgagccagtgaccgtgtcgtggaacagcggagccctgacttccggcgtgcacacttttcccgcggtgctgcagt |
| | | cctccggtctgtactccctttcgtccgtggtcaccgtgccgtcgtctagcctgggcacccagacctacatctgcaacgtg |
| | | aaccacaagccgtccaacaccaaagtggataagcgggtggagccgaagtcctgcgataagacacacacgtgcccgc |
| | | catgtccagcgcctgaattgcttggcggaccttccgtgttcctgttcccgcctaagcccaaggacaccttgatgattagcc |
| | | ggactcccgaagtcacctgtgtggtggtggcagtgtcccacgaggaccccgaggtcaagtttaattggtacgtggacg |
| | | gcgtcgaagtgcacaacgccaagactaagccccgggaggaacagtacaacagcacctaccgggtcgtgtccgtgct |
| | | gaccgtgctgcaccaggactggctgaatgggaaagagtacaagtgcaaagtgtccaacaaggccttggccgctcctat |
| | | cgaaaaaactatcagcaaggctaagggacagccgagggaaccccaagtctacaccctgcccccttcacgcgaagag |
| | | atgaccaagaatcaagtgtcgctgacctgcctcgtcaagggattctacccctccgacattgcggtggagtgggagtcca |
| | | acggccagcccgagaacaactacaagactactccgcccgtgctggactccgacggcagcttcttcctgtattccaagct |
| | | gaccgtggacaagtcccggtggcagcaaggaaacgtgttctcctgctcggtcatgcacgaagccctgcacaaccacta |
| | | tacgcagaagtccctgtccttgagcccggggaaa |
| SEQ ID NO: 41 | LCDR1 (Combined) | RASQGISSYLA |
| SEQ ID NO: 42 | LCDR2 (Combined) | TASTLQS |
| SEQ ID NO: 134 | LCDR3 (Combined) | QQTWRKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 134 | LCDR3 (Kabat) | QQTWRKPRT |
| SEQ ID NO: 44 | LCDR1 (Chothia) | SQGISSY |
| SEQ ID NO: 45 | LCDR2 (Chothia) | TAS |
| SEQ ID NO: 135 | LCDR3 (Chothia) | TWRKPR |
| SEQ ID NO: 47 | LCDR1 (IMGT) | QGISSY |
| SEQ ID NO: 45 | LCDR2 (IMGT) | TAS |
| SEQ ID NO: 134 | LCDR3 (IMGT) | QQTWRKPRT |
| SEQ ID NO: 136 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIK |
| SEQ ID NO: 143 | DNA VL | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaacctggcggaagcccaggacatttggccagggcactaaggtcga |
| | | gattaag |
| SEQ ID NO: 138 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 144 | DNA Light Chain | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaacctggcggaagcccaggacatttggccagggcactaaggtcga |
| | | gattaagcgtacggtggccgctcccagcgtgttcatcttcccccccagcgacgagcagctgaagagcggcaccgcca |
| | | gcgtggtgtgcctgctgaacaacttctacccccgggaggccaaggtgcagtggaaggtggacaacgccctgcagag |
| | | cggcaacagccaggagagcgtcaccgagcaggacagcaaggactccacctacagcctgagcagcaccctgaccct |
| | | gagcaaggccgactacgagaagcataaggtgtacgcctgcgaggtgacccaccagggcctgtccagccccgtgacc |
| | | aagagcttcaacaggggcgagtgc |
| XX07_LALA | | |
| SEQ ID NO: 28 | HCDR1 (Combined) | GFTFSSYWMN |
| SEQ ID NO: 29 | HCDR2 (Combined) | AISSDGSYTYYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 29 | HCDR2 (Kabat) | AISSDGSYTYYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 33 | HCDR2 (Chothia) | SSDGSY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 35 | HCDR2 (IMGT) | ISSDGSYT |
| SEQ ID NO: 36 | HCDR3 (IMGT) | ARDRYSMIYSYGAGAFDY |
| SEQ ID NO: 37 | VH | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSS |
| SEQ ID NO: 38 | DNA VH | caggtgcaattgctggaaagcggcggtggcctggtgcagccgggtggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctca |
| SEQ ID NO: 39 | Heavy Chain | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 40 | DNA Heavy Chain | caggtgcaattgctggaaagcggcggtggcctggtgcagccgggtggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctcagcctccaccaagggtccatcggt |
| | | cttccccctggcaccctcctccaagagcacctctgggggcacagcggccctgggctgcctggtcaaggactacttccc |
| | | cgaaccggtgacggtgtcgtggaactcaggcgccctgaccagcggcgtgcacaccttcccggctgtcctacagtcctc |
| | | aggactctactccctcagcagcgtggtgaccgtgccctccagcagcttgggcacccagacctacatctgcaacgtgaat |
| | | cacaagcccagcaacaccaaggtggacaagagagttgagcccaaatcttgtgacaaaactcacacatgcccaccgtg |
| | | cccagcacctgaagcagcggggggaccgtcagtcttcctcttccccccaaaacccaaggacaccctcatgatctcccg |
| | | gacccctgaggtcacatgcgtggtggtggacgtgagccacgaagaccctgaggtcaagttcaactggtacgtggacg |
| | | gcgtggaggtgcataatgccaagacaaagccgcgggaggagcagtacaacagcacgtaccgggtggtcagcgtcct |
| | | caccgtcctgcaccaggactggctgaatggcaaggagtacaagtgcaaggtctccaacaaagccctcccagccccca |
| | | tcgagaaaaccatctccaaagccaaagggcagccccgagaaccacaggtgtacaccctgcccccatcccgggagga |
| | | gatgaccaagaaccaggtcagcctgacctgcctggtcaaaggcttctatcccagcgacatcgccgtggagtgggaga |
| | | gcaatgggcagccggagaacaactacaagaccacgcctcccgtgctggactccgacggctccttcttcctctacagca |
| | | agctcaccgtggacaagagcaggtggcagcaggggaacgtcttctcatgctccgtgatgcatgaggctctgcacaacc |
| | | actacacgcagaagagcctctccctgtctccgggtaaa |
| SEQ ID NO: 41 | LCDR1 (Combined) | RASQGISSYLA |
| SEQ ID NO: 42 | LCDR2 (Combined) | TASTLQS |
| SEQ ID NO: 145 | LCDR3 (Combined) | QQIWTVPRT |
| SEQ ID NO: 41 | LCDR1 (Kabat) | RASQGISSYLA |
| SEQ ID NO: 42 | LCDR2 (Kabat) | TASTLQS |
| SEQ ID NO: 145 | LCDR3 (Kabat) | QQIWTVPRT |
| SEQ ID NO: 44 | LCDR1 (Chothia) | SQGISSY |
| SEQ ID NO: 45 | LCDR2 (Chothia) | TAS |
| SEQ ID NO: 146 | LCDR3 (Chothia) | IWTVPR |
| SEQ ID NO: 47 | LCDR1 (IMGT) | QGISSY |
| SEQ ID NO: 45 | LCDR2 (IMGT) | TAS |
| SEQ ID NO: 145 | LCDR3 (IMGT) | QQIWTVPRT |
| SEQ ID NO: 147 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWTVPRTFGQGTKV |
| | | EIK |
| SEQ ID NO: 148 | DNA VL | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcagatctggactgttccgcgtacctttggccagggcacgaaagttgaaa |
| | | ttaaa |
| SEQ ID NO: 149 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWTVPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 150 | DNA Light Chain | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcagatctggactgttccgcgtacctttggccagggcacgaaagttgaaa |
| | | ttaaacgtacggtggccgctcccagcgtgttcatcttcccccccagcgacgagcagctgaagagcggcaccgccagc |
| | | gtggtgtgcctgctgaacaacttctacccccgggaggccaaggtgcagtggaaggtggacaacgccctgcagagcg |
| | | gcaacagccaggaaagcgtcaccgagcaggacagcaaggactccacctacagcctgagcagcaccctgaccctga |
| | | gcaaggccgactacgagaagcacaaggtgtacgcctgcgaggtgacccaccagggcctgtccagccccgtgacca |
| | | agagcttcaaccggggcgagtgt |
| XX08_LALA | | |
| SEQ ID NO: 4 | HCDR1 (Combined) | GFTFNTHYIH |
| SEQ ID NO: 151 | HCDR2 (Combined) | SIGGQGGMTLYADSVKG |
| SEQ ID NO: 6 | HCDR3 (Combined) | ERGYVYYHMFDP |
| SEQ ID NO: 7 | HCDR1 (Kabat) | THYIH |
| SEQ ID NO: 151 | HCDR2 (Kabat) | SIGGQGGMTLYADSVKG |
| SEQ ID NO: 6 | HCDR3 (Kabat) | ERGYVYYHMFDP |
| SEQ ID NO: 8 | HCDR1 (Chothia) | GFTFNTH |
| SEQ ID NO: 152 | HCDR2 (Chothia) | GGQGGM |
| SEQ ID NO: 6 | HCDR3 (Chothia) | ERGYVYYHMFDP |
| SEQ ID NO: 10 | HCDR1 (IMGT) | GFTFNTHY |
| SEQ ID NO: 153 | HCDR2 (IMGT) | IGGQGGMT |
| SEQ ID NO: 12 | HCDR3 (IMGT) | ARERGYVYYHMFDP |
| SEQ ID NO: 154 | VH | EVQLLESGGGLVQPGGSLRLSCAASGFTFNTHYIHWVRQAPGKGLEWVSSI |
| | | GGQGGMTLYADSVKGRFTISRDNSKNTLYLQMNSLRAEDTAVYYCARER |
| | | GYVYYHMFDPWGQGTLVTVSS |
| SEQ ID NO: 155 | DNA VH | gaggtgcaattgctggaaagcggcggtggcctggtgcagccgggggcagcctgcgtctgagctgcgcggcgtccg |
| | | gattcacctttaacactcattacatccattgggtgcgccaggcccccggcaaaggtctcgagtgggtttcctctatcggtg |
| | | gtcagggcggtatgactctgtatgcggatagcgtgaaaggccgctttaccatcagccgcgataattcgaaaaacaccct |
| | | gtatctgcaaatgaacagcctgcgtgcggaagatacggccgtgtattattgcgcgcgtgaacgtggttacgtttactacc |
| | | atatgttcgatccgtggggccaaggcaccctggtgactgttagctca |
| SEQ ID NO: 156 | Heavy Chain | EVQLLESGGGLVQPGGSLRLSCAASGFTFNTHYIHWVRQAPGKGLEWVSSI |
| | | GGQGGMTLYADSVKGRFTISRDNSKNTLYLQMNSLRAEDTAVYYCARER |
| | | GYVYYHMFDPWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTY |
| | | ICNVNHKPSNTKVDKRVEPKSCDKTHTCPPCPAPEAAGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 157 | DNA Heavy Chain | gaggtgcaattgctggaaagcggcggtggcctggtgcagccgggtggcagcctgcgtctgagctgcgcggcgtccg |
| | | gattcacctttaacactcattacatccattgggtgcgccaggcccccggcaaaggtctcgagtgggtttcctctatcggtg |
| | | gtcagggcggtatgactctgtatgcggatagcgtgaaaggccgctttaccatcagccgcgataattcgaaaaacaccct |
| | | gtatctgcaaatgaacagcctgcgtgcggaagatacggccgtgtattattgcgcgcgtgaacgtggttacgtttactacc |
| | | atatgttcgatccgtggggccaaggcaccctggtgactgttagctcagcctccaccaagggtccatcggtcttccccctg |
| | | gcaccctcctccaagagcacctctgggggcacagcggccctgggctgcctggtcaaggactacttccccgaaccggt |
| | | gacggtgtcgtggaactcaggcgccctgaccagcggcgtgcacaccttcccggctgtcctacagtcctcaggactcta |
| | | ctccctcagcagcgtggtgaccgtgccctccagcagcttgggcacccagacctacatctgcaacgtgaatcacaagcc |
| | | cagcaacaccaaggtggacaagagagttgagcccaaatcttgtgacaaaactcacacatgcccaccgtgcccagcac |
| | | ctgaagcagcggggggaccgtcagtcttcctcttccccccaaaacccaaggacaccctcatgatctcccggacccctg |
| | | aggtcacatgcgtggtggtggacgtgagccacgaagaccctgaggtcaagttcaactggtacgtggacggcgtggag |
| | | gtgcataatgccaagacaaagccgcgggaggagcagtacaacagcacgtaccgggtggtcagcgtcctcaccgtcct |
| | | gcaccaggactggctgaatggcaaggagtacaagtgcaaggtctccaacaaagccctcccagcccccatcgagaaa |
| | | accatctccaaagccaaagggcagccccgagaaccacaggtgtacaccctgcccccatcccgggaggagatgacca |
| | | agaaccaggtcagcctgacctgcctggtcaaaggcttctatcccagcgacatcgccgtggagtgggagagcaatggg |
| | | cagccggagaacaactacaagaccacgcctcccgtgctggactccgacggctccttcttcctctacagcaagctcacc |
| | | gtggacaagagcaggtggcagcaggggaacgtcttctcatgctccgtgatgcatgaggctctgcacaaccactacacg |
| | | cagaagagcctctccctgtctccgggtaaa |
| SEQ ID NO: 17 | LCDR1 (Combined) | RASQSITRNYLA |
| SEQ ID NO: 18 | LCDR2 (Combined) | GASSRAT |
| SEQ ID NO: 19 | LCDR3 (Combined) | QQHSMYPRT |
| SEQ ID NO: 17 | LCDR1 (Kabat) | RASQSITRNYLA |
| SEQ ID NO: 18 | LCDR2 (Kabat) | GASSRAT |
| SEQ ID NO: 19 | LCDR3 (Kabat) | QQHSMYPRT |
| SEQ ID NO: 20 | LCDR1 (Chothia) | SQSITRNY |
| SEQ ID NO: 21 | LCDR2 (Chothia) | GAS |
| SEQ ID NO: 22 | LCDR3 (Chothia) | HSMYPR |
| SEQ ID NO: 23 | LCDR1 (IMGT) | QSITRNY |
| SEQ ID NO: 21 | LCDR2 (IMGT) | GAS |
| SEQ ID NO: 19 | LCDR3 (IMGT) | QQHSMYPRT |
| SEQ ID NO: 24 | VL | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIK |
| SEQ ID NO: 25 | DNA VL | gatatcgtgctgacccagagcccggcgaccctgagcctgagcccgggtgaacgtgccaccctgagctgcagagcga |
| | | gccagtctatcactcgtaactacctggcttggtaccagcagaaaccgggccaggccccgcgtctattaatctacggtgct |
| | | tcttctcgtgcgaccggcattccggcgcgttttagcggcagcggatccggcaccgatttcaccctgaccattagcagcct |
| | | ggaaccggaagactttgcggtgtattattgccagcagcattctatgtacccgcgtacctttggccagggcacgaaagttg |
| | | aaattaaa |
| SEQ ID NO: 26 | Light Chain | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| SEQ ID NO: 27 | DNA Light Chain | gatatcgtgctgacccagagcccggcgaccctgagcctgagcccgggtgaacgtgccaccctgagctgcagagcga |
| | | gccagtctatcactcgtaactacctggcttggtaccagcagaaaccgggccaggccccgcgtctattaatctacggtgct |
| | | tcttctcgtgcgaccggcattccggcgcgttttagcggcagcggatccggcaccgatttcaccctgaccattagcagcct |
| | | ggaaccggaagactttgcggtgtattattgccagcagcattctatgtacccgcgtacctttggccagggcacgaaagttg |
| | | aaattaaacgtacggtggccgctcccagcgtgttcatcttcccccccagcgacgagcagctgaagagcggcaccgcc |
| | | agcgtggtgtgcctgctgaacaacttctacccccgggaggccaaggtgcagtggaaggtggacaacgccctgcaga |
| | | gcggcaacagccaggaaagcgtcaccgagcaggacagcaaggactccacctacagcctgagcagcaccctgaccc |
| | | tgagcaaggccgactacgagaagcacaaggtgtacgcctgcgaggtgacccaccagggcctgtccagccccgtgac |
| | | caagagcttcaaccggggcgagtgt |
| XX08_DAPA | | |
| SEQ ID NO: 4 | HCDR1 (Combined) | GFTFNTHYIH |
| SEQ ID NO: 151 | HCDR2 (Combined) | SIGGQGGMTLYADSVKG |
| SEQ ID NO: 6 | HCDR3 (Combined) | ERGYVYYHMFDP |
| SEQ ID NO: 7 | HCDR1 (Kabat) | THYIH |
| SEQ ID NO: 151 | HCDR2 (Kabat) | SIGGQGGMTLYADSVKG |
| SEQ ID NO: 6 | HCDR3 (Kabat) | ERGYVYYHMFDP |
| SEQ ID NO: 8 | HCDR1 (Chothia) | GFTFNTH |
| SEQ ID NO: 152 | HCDR2 (Chothia) | GGQGGM |
| SEQ ID NO: 6 | HCDR3 (Chothia) | ERGYVYYHMFDP |
| SEQ ID NO: 10 | HCDR1 (IMGT) | GFTFNTHY |
| SEQ ID NO: 153 | HCDR2 (IMGT) | IGGQGGMT |
| SEQ ID NO: 12 | HCDR3 (IMGT) | ARERGYVYYHMFDP |
| SEQ ID NO: 154 | VH | EVQLLESGGGLVQPGGSLRLSCAASGFTFNTHYIHWVRQAPGKGLEWVSSI |
| | | GGQGGMTLYADSVKGRFTISRDNSKNTLYLQMNSLRAEDTAVYYCARER |
| | | GYVYYHMFDPWGQGTLVTVSS |
| SEQ ID NO: 158 | DNA VH | gaagtgcagctcctggagtcgggtggcggactggtgcagcctggcggatcactgcggctgtcatgtgccgcgagcg |
| | | ggtttactttcaacacccactacatccactgggtccgccaagctcccggaaagggactcgaatgggtgtcctccattggt |
| | | ggacagggcggcatgaccctttacgcggatagcgtgaaggggaggttcaccatctcccgcgacaacagcaagaaca |
| | | ccctgtacctccaaatgaactcgcttcgggccgaggacactgccgtgtactattgcgcaagagagcggggctacgtgt |
| | | actaccacatgttcgacccatggggacagggaacgctggtcaccgtgtcctcc |
| SEQ ID NO: 159 | Heavy Chain | EVQLLESGGGLVQPGGSLRLSCAASGFTFNTHYIHWVRQAPGKGLEWVSSI |
| | | GGQGGMTLYADSVKGRFTISRDNSKNTLYLQMNSLRAEDTAVYYCARER |
| | | GYVYYHMFDPWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTY |
| | | ICNVNHKPSNTKVDKRVEPKSCDKTHTCPPCPAPELLGGPSVFLFPPKPKDT |
| | | LMISRTPEVTCVVVAVSHEDPEVKFNWYVDGVEVHNAKTKPREEQYNSTY |
| | | RVVSVLTVLHQDWLNGKEYKCKVSNKALAAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 160 | DNA Heavy Chain | gaagtgcagctcctggagtcgggtggcggactggtgcagcctggcggatcactgcggctgtcatgtgccgcgagcg |
| | | ggtttactttcaacacccactacatccactgggtccgccaagctcccggaaagggactcgaatgggtgtcctccattggt |
| | | ggacagggcggcatgaccctttacgcggatagcgtgaaggggaggttcaccatctcccgcgacaacagcaagaaca |
| | | ccctgtacctccaaatgaactcgcttcgggccgaggacactgccgtgtactattgcgcaagagagcggggctacgtgt |
| | | actaccacatgttcgacccatggggacagggaacgctggtcaccgtgtcctccgcctccactaagggcccgtcagtgtt |
| | | cccgctggctccatcgtcgaagtccacctccggaggaaccgcagcactcggttgcctggtcaaggactacttccctga |
| | | gccagtgaccgtgtcgtggaacagcggagccctgacttccggcgtgcacacttttcccgcggtgctgcagtcctccggt |
| | | ctgtactccctttcgtccgtggtcaccgtgccgtcgtctagcctgggcacccagacctacatctgcaacgtgaaccacaa |
| | | gccgtccaacaccaaagtggataagcgggtggagccgaagtcctgcgataagacacacacgtgcccgccatgtcca |
| | | gcgcctgaattgcttggcggaccttccgtgttcctgttcccgcctaagcccaaggacaccttgatgattagccggactcc |
| | | cgaagtcacctgtgtggtggtggcagtgtcccacgaggaccccgaggtcaagtttaattggtacgtggacggcgtcga |
| | | agtgcacaacgccaagactaagccccgggaggaacagtacaacagcacctaccgggtcgtgtccgtgctgaccgtg |
| | | ctgcaccaggactggctgaatgggaaagagtacaagtgcaaagtgtccaacaaggccttggccgctcctatcgaaaaa |
| | | actatcagcaaggctaagggacagccgagggaaccccaagtctacaccctgcccccttcacgcgaagagatgacca |
| | | agaatcaagtgtcgctgacctgcctcgtcaagggattctacccctccgacattgcggtggagtgggagtccaacggcc |
| | | agcccgagaacaactacaagactactccgcccgtgctggactccgacggcagcttcttcctgtattccaagctgaccgt |
| | | ggacaagtcccggtggcagcaaggaaacgtgttctcctgctcggtcatgcacgaagccctgcacaaccactatacgca |
| | | gaagtccctgtccttgagcccggggaaa |
| SEQ ID NO: 17 | LCDR1 (Combined) | RASQSITRNYLA |
| SEQ ID NO: 18 | LCDR2 (Combined) | GASSRAT |
| SEQ ID NO: 19 | LCDR3 (Combined) | QQHSMYPRT |
| SEQ ID NO: 17 | LCDR1 (Kabat) | RASQSITRNYLA |
| SEQ ID NO: 18 | LCDR2 (Kabat) | GASSRAT |
| SEQ ID NO: 19 | LCDR3 (Kabat) | QQHSMYPRT |
| SEQ ID NO: 20 | LCDR1 (Chothia) | SQSITRNY |
| SEQ ID NO: 21 | LCDR2 (Chothia) | GAS |
| SEQ ID NO: 22 | LCDR3 (Chothia) | HSMYPR |
| SEQ ID NO: 23 | LCDR1 (IMGT) | QSITRNY |
| SEQ ID NO: 21 | LCDR2 (IMGT) | GAS |
| SEQ ID NO: 19 | LCDR3 (IMGT) | QQHSMYPRT |
| SEQ ID NO: 24 | VL | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIK |
| SEQ ID NO: 110 | DNA VL | gacatcgtgctgactcagtcccctgcgactctgagcctgtcaccgggagaacgggccaccctctcttgccgcgcctcc |
| | | caatccattactcggaactacctggcctggtatcagcagaagccaggacaggcccctaggcttctgatctacggggcc |
| | | agctcaagagcaactggcatcccggctcgcttctccggttcgggaagcggcaccgacttcaccctgacaatttcgtccc |
| | | tcgaacccgaggatttcgccgtgtactactgccaacagcactccatgtacccccggacctttgggcagggaaccaaagt |
| | | cgagatcaag |
| SEQ ID NO: 26 | Light Chain | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| SEQ ID NO: 111 | DNA Light Chain | gacatcgtgctgactcagtcccctgcgactctgagcctgtcaccgggagaacgggccaccctctcttgccgcgcctcc |
| | | caatccattactcggaactacctggcctggtatcagcagaagccaggacaggcccctaggcttctgatctacggggcc |
| | | agctcaagagcaactggcatcccggctcgcttctccggttcgggaagcggcaccgacttcaccctgacaatttcgtccc |
| | | tcgaacccgaggatttcgccgtgtactactgccaacagcactccatgtacccccggacctttgggcagggaaccaaagt |
| | | cgagatcaagcgtacggtggccgctcccagcgtgttcatcttcccccccagcgacgagcagctgaagagcggcaccg |
| | | ccagcgtggtgtgcctgctgaacaacttctacccccgggaggccaaggtgcagtggaaggtggacaacgccctgcag |
| | | agcggcaacagccaggagagcgtcaccgagcaggacagcaaggactccacctacagcctgagcagcaccctgacc |
| | | ctgagcaaggccgactacgagaagcataaggtgtacgcctgcgaggtgacccaccagggcctgtccagccccgtga |
| | | ccaagagcttcaacaggggcgagtgc |
| XX08_N30S_DAPA | | |
| SEQ ID NO: 112 | HCDR1 (Combined) | GFTFSTHYIH |
| SEQ ID NO: 151 | HCDR2 (Combined) | SIGGQGGMTLYADSVKG |
| SEQ ID NO: 6 | HCDR3 (Combined) | ERGYVYYHMFDP |
| SEQ ID NO: 7 | HCDR1 (Kabat) | THYIH |
| SEQ ID NO: 151 | HCDR2 (Kabat) | SIGGQGGMTLYADSVKG |
| SEQ ID NO: 6 | HCDR3 (Kabat) | ERGYVYYHMFDP |
| SEQ ID NO: 113 | HCDR1 (Chothia) | GFTFSTH |
| SEQ ID NO: 152 | HCDR2 (Chothia) | GGQGGM |
| SEQ ID NO: 6 | HCDR3 (Chothia) | ERGYVYYHMFDP |
| SEQ ID NO: 114 | HCDR1 (IMGT) | GFTFSTHY |
| SEQ ID NO: 153 | HCDR2 (IMGT) | IGGQGGMT |
| SEQ ID NO: 12 | HCDR3 (IMGT) | ARERGYVYYHMFDP |
| SEQ ID NO: 161 | VH | EVQLLESGGGLVQPGGSLRLSCAASGFTFSTHYIHWVRQAPGKGLEWVSSI |
| | | GGQGGMTLYADSVKGRFTISRDNSKNTLYLQMNSLRAEDTAVYYCARER |
| | | GYVYYHMFDPWGQGTLVTVSS |
| SEQ ID NO: 162 | DNA VH | gaagtgcagctcctggagtcgggtggcggactggtgcagcctggcggatcactgcggctgtcatgtgccgcgagcg |
| | | ggtttactttctccacccactacatccactgggtccgccaagctcccggaaagggactcgaatgggtgtcctccattggt |
| | | ggacagggcggcatgaccctttacgcggatagcgtgaaggggaggttcaccatctcccgcgacaacagcaagaaca |
| | | ccctgtacctccaaatgaactcgcttcgggccgaggacactgccgtgtactattgcgcaagagagcggggctacgtgt |
| | | actaccacatgttcgacccatggggacagggaacgctggtcaccgtgtcctcc |
| SEQ ID NO: 163 | Heavy Chain | EVQLLESGGGLVQPGGSLRLSCAASGFTFSTHYIHWVRQAPGKGLEWVSSI |
| | | GGQGGMTLYADSVKGRFTISRDNSKNTLYLQMNSLRAEDTAVYYCARER |
| | | GYVYYHMFDPWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTY |
| | | ICNVNHKPSNTKVDKRVEPKSCDKTHTCPPCPAPELLGGPSVFLFPPKPKDT |
| | | LMISRTPEVTCVVVAVSHEDPEVKFNWYVDGVEVHNAKTKPREEQYNSTY |
| | | RVVSVLTVLHQDWLNGKEYKCKVSNKALAAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 164 | DNA Heavy Chain | gaagtgcagctcctggagtcgggtggcggactggtgcagcctggcggatcactgcggctgtcatgtgccgcgagcg |
| | | ggtttactttctccacccactacatccactgggtccgccaagctcccggaaagggactcgaatgggtgtcctccattggt |
| | | ggacagggcggcatgaccctttacgcggatagcgtgaaggggaggttcaccatctcccgcgacaacagcaagaaca |
| | | ccctgtacctccaaatgaactcgcttcgggccgaggacactgccgtgtactattgcgcaagagagcggggctacgtgt |
| | | actaccacatgttcgacccatggggacagggaacgctggtcaccgtgtcctccgcctccactaagggcccgtcagtgtt |
| | | cccgctggctccatcgtcgaagtccacctccggaggaaccgcagcactcggttgcctggtcaaggactacttccctga |
| | | gccagtgaccgtgtcgtggaacagcggagccctgacttccggcgtgcacacttttcccgcggtgctgcagtcctccggt |
| | | ctgtactccctttcgtccgtggtcaccgtgccgtcgtctagcctgggcacccagacctacatctgcaacgtgaaccacaa |
| | | gccgtccaacaccaaagtggataagcgggtggagccgaagtcctgcgataagacacacacgtgcccgccatgtcca |
| | | gcgcctgaattgcttggcggaccttccgtgttcctgttcccgcctaagcccaaggacaccttgatgattagccggactcc |
| | | cgaagtcacctgtgtggtggtggcagtgtcccacgaggaccccgaggtcaagtttaattggtacgtggacggcgtcga |
| | | agtgcacaacgccaagactaagccccgggaggaacagtacaacagcacctaccgggtcgtgtccgtgctgaccgtg |
| | | ctgcaccaggactggctgaatgggaaagagtacaagtgcaaagtgtccaacaaggccttggccgctcctatcgaaaaa |
| | | actatcagcaaggctaagggacagccgagggaaccccaagtctacaccctgcccccttcacgcgaagagatgacca |
| | | agaatcaagtgtcgctgacctgcctcgtcaagggattctacccctccgacattgcggtggagtgggagtccaacggcc |
| | | agcccgagaacaactacaagactactccgcccgtgctggactccgacggcagcttcttcctgtattccaagctgaccgt |
| | | ggacaagtcccggtggcagcaaggaaacgtgttctcctgctcggtcatgcacgaagccctgcacaaccactatacgca |
| | | gaagtccctgtccttgagcccggggaaa |
| SEQ ID NO: 17 | LCDR1 (Combined) | RASQSITRNYLA |
| SEQ ID NO: 18 | LCDR2 (Combined) | GASSRAT |
| SEQ ID NO: 19 | LCDR3 (Combined) | QQHSMYPRT |
| SEQ ID NO: 17 | LCDR1 (Kabat) | RASQSITRNYLA |
| SEQ ID NO: 18 | LCDR2 (Kabat) | GASSRAT |
| SEQ ID NO: 19 | LCDR3 (Kabat) | QQHSMYPRT |
| SEQ ID NO: 20 | LCDR1 (Chothia) | SQSITRNY |
| SEQ ID NO: 21 | LCDR2 (Chothia) | GAS |
| SEQ ID NO: 22 | LCDR3 (Chothia) | HSMYPR |
| SEQ ID NO: 23 | LCDR1 (IMGT) | QSITRNY |
| SEQ ID NO: 21 | LCDR2 (IMGT) | GAS |
| SEQ ID NO: 19 | LCDR3 (IMGT) | QQHSMYPRT |
| SEQ ID NO: 24 | VL | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIK |
| SEQ ID NO: 110 | DNA VL | gacatcgtgctgactcagtcccctgcgactctgagcctgtcaccgggagaacgggccaccctctcttgccgcgcctcc |
| | | caatccattactcggaactacctggcctggtatcagcagaagccaggacaggcccctaggcttctgatctacggggcc |
| | | agctcaagagcaactggcatcccggctcgcttctccggttcgggaagcggcaccgacttcaccctgacaatttcgtccc |
| | | tcgaacccgaggatttcgccgtgtactactgccaacagcactccatgtacccccggacctttgggcagggaaccaaagt |
| | | cgagatcaag |
| SEQ ID NO: 26 | Light Chain | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| SEQ ID NO: 111 | DNA Light Chain | gacatcgtgctgactcagtcccctgcgactctgagcctgtcaccgggagaacgggccaccctctcttgccgcgcctcc |
| | | caatccattactcggaactacctggcctggtatcagcagaagccaggacaggcccctaggcttctgatctacggggcc |
| | | agctcaagagcaactggcatcccggctcgcttctccggttcgggaagcggcaccgacttcaccctgacaatttcgtccc |
| | | tcgaacccgaggatttcgccgtgtactactgccaacagcactccatgtacccccggacctttgggcagggaaccaaagt |
| | | cgagatcaagcgtacggtggccgctcccagcgtgttcatcttcccccccagcgacgagcagctgaagagcggcaccg |
| | | ccagcgtggtgtgcctgctgaacaacttctacccccgggaggccaaggtgcagtggaaggtggacaacgccctgcag |
| | | agcggcaacagccaggagagcgtcaccgagcaggacagcaaggactccacctacagcctgagcagcaccctgacc |
| | | ctgagcaaggccgactacgagaagcataaggtgtacgcctgcgaggtgacccaccagggcctgtccagccccgtga |
| | | ccaagagcttcaacaggggcgagtgc |
| XX08_N30Q DAPA | | |
| SEQ ID NO: 165 | HCDR1 (Combined) | GFTFQTHYIH |
| SEQ ID NO: 151 | HCDR2 (Combined) | SIGGQGGMTLYADSVKG |
| SEQ ID NO: 6 | HCDR3 (Combined) | ERGYVYYHMFDP |
| SEQ ID NO: 7 | HCDR1 (Kabat) | THYIH |
| SEQ ID NO: 151 | HCDR2 (Kabat) | SIGGQGGMTLYADSVKG |
| SEQ ID NO: 6 | HCDR3 (Kabat) | ERGYVYYHMFDP |
| SEQ ID NO: 166 | HCDR1 (Chothia) | GFTFQTH |
| SEQ ID NO: 152 | HCDR2 (Chothia) | GGQGGM |
| SEQ ID NO: 6 | HCDR3 (Chothia) | ERGYVYYHMFDP |
| SEQ ID NO: 167 | HCDR1 (IMGT) | GFTFQTHY |
| SEQ ID NO: 153 | HCDR2 (IMGT) | IGGQGGMT |
| SEQ ID NO: 12 | HCDR3 (IMGT) | ARERGYVYYHMFDP |
| SEQ ID NO: 168 | VH | EVQLLESGGGLVQPGGSLRLSCAASGFTFQTHYIHWVRQAPGKGLEWVSSI |
| | | GGQGGMTLYADSVKGRFTISRDNSKNTLYLQMNSLRAEDTAVYYCARER |
| | | GYVYYHMFDPWGQGTLVTVSS |
| SEQ ID NO: 169 | DNA VH | gaagtgcagctcctggagtcgggtggcggactggtgcagcctggcggatcactgcggctgtcatgtgccgcgagcg |
| | | ggtttactttccagacccactacatccactgggtccgccaagctcccggaaagggactcgaatgggtgtcctccattggt |
| | | ggacagggcggcatgaccctttacgcggatagcgtgaaggggaggttcaccatctcccgcgacaacagcaagaaca |
| | | ccctgtacctccaaatgaactcgcttcgggccgaggacactgccgtgtactattgcgcaagagagcggggctacgtgt |
| | | actaccacatgttcgacccatggggacagggaacgctggtcaccgtgtcctcc |
| SEQ ID NO: 170 | Heavy Chain | EVQLLESGGGLVQPGGSLRLSCAASGFTFQTHYIHWVRQAPGKGLEWVSSI |
| | | GGQGGMTLYADSVKGRFTISRDNSKNTLYLQMNSLRAEDTAVYYCARER |
| | | GYVYYHMFDPWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTY |
| | | ICNVNHKPSNTKVDKRVEPKSCDKTHTCPPCPAPELLGGPSVFLFPPKPKDT |
| | | LMISRTPEVTCVVVAVSHEDPEVKFNWYVDGVEVHNAKTKPREEQYNSTY |
| | | RVVSVLTVLHQDWLNGKEYKCKVSNKALAAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 171 | DNA Heavy Chain | gaagtgcagctcctggagtcgggtggcggactggtgcagcctggcggatcactgcggctgtcatgtgccgcgagcg |
| | | ggtttactttccagacccactacatccactgggtccgccaagctcccggaaagggactcgaatgggtgtcctccattggt |
| | | ggacagggcggcatgaccctttacgcggatagcgtgaaggggaggttcaccatctcccgcgacaacagcaagaaca |
| | | ccctgtacctccaaatgaactcgcttcgggccgaggacactgccgtgtactattgcgcaagagagcggggctacgtgt |
| | | actaccacatgttcgacccatggggacagggaacgctggtcaccgtgtcctccgcctccactaagggcccgtcagtgtt |
| | | cccgctggctccatcgtcgaagtccacctccggaggaaccgcagcactcggttgcctggtcaaggactacttccctga |
| | | gccagtgaccgtgtcgtggaacagcggagccctgacttccggcgtgcacacttttcccgcggtgctgcagtcctccggt |
| | | ctgtactccctttcgtccgtggtcaccgtgccgtcgtctagcctgggcacccagacctacatctgcaacgtgaaccacaa |
| | | gccgtccaacaccaaagtggataagcgggtggagccgaagtcctgcgataagacacacacgtgcccgccatgtcca |
| | | gcgcctgaattgcttggcggaccttccgtgttcctgttcccgcctaagcccaaggacaccttgatgattagccggactcc |
| | | cgaagtcacctgtgtggtggtggcagtgtcccacgaggaccccgaggtcaagtttaattggtacgtggacggcgtcga |
| | | agtgcacaacgccaagactaagccccgggaggaacagtacaacagcacctaccgggtcgtgtccgtgctgaccgtg |
| | | ctgcaccaggactggctgaatgggaaagagtacaagtgcaaagtgtccaacaaggccttggccgctcctatcgaaaaa |
| | | actatcagcaaggctaagggacagccgagggaaccccaagtctacaccctgcccccttcacgcgaagagatgacca |
| | | agaatcaagtgtcgctgacctgcctcgtcaagggattctacccctccgacattgcggtggagtgggagtccaacggcc |
| | | agcccgagaacaactacaagactactccgcccgtgctggactccgacggcagcttcttcctgtattccaagctgaccgt |
| | | ggacaagtcccggtggcagcaaggaaacgtgttctcctgctcggtcatgcacgaagccctgcacaaccactatacgca |
| | | gaagtccctgtccttgagcccggggaaa |
| SEQ ID NO: 17 | LCDR1 (Combined) | RASQSITRNYLA |
| SEQ ID NO: 18 | LCDR2 (Combined) | GASSRAT |
| SEQ ID NO: 19 | LCDR3 (Combined) | QQHSMYPRT |
| SEQ ID NO: 17 | LCDR1 (Kabat) | RASQSITRNYLA |
| SEQ ID NO: 18 | LCDR2 (Kabat) | GASSRAT |
| SEQ ID NO: 19 | LCDR3 (Kabat) | QQHSMYPRT |
| SEQ ID NO: 20 | LCDR1 (Chothia) | SQSITRNY |
| SEQ ID NO: 21 | LCDR2 (Chothia) | GAS |
| SEQ ID NO: 22 | LCDR3 (Chothia) | HSMYPR |
| SEQ ID NO: 23 | LCDR1 (IMGT) | QSITRNY |
| SEQ ID NO: 21 | LCDR2 (IMGT) | GAS |
| SEQ ID NO: 19 | LCDR3 (IMGT) | QQHSMYPRT |
| SEQ ID NO: 24 | VL | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIK |
| SEQ ID NO: 110 | DNA VL | gacatcgtgctgactcagtcccctgcgactctgagcctgtcaccgggagaacgggccaccctctcttgccgcgcctcc |
| | | caatccattactcggaactacctggcctggtatcagcagaagccaggacaggcccctaggcttctgatctacggggcc |
| | | agctcaagagcaactggcatcccggctcgcttctccggttcgggaagcggcaccgacttcaccctgacaatttcgtccc |
| | | tcgaacccgaggatttcgccgtgtactactgccaacagcactccatgtacccccggacctttgggcagggaaccaaagt |
| | | cgagatcaag |
| SEQ ID NO: 26 | Light Chain | DIVLTQSPATLSLSPGERATLSCRASQSITRNYLAWYQQKPGQAPRLLIYGA |
| | | SSRATGIPARFSGSGSGTDFTLTISSLEPEDFAVYYCQQHSMYPRTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| SEQ ID NO: 111 | DNA Light Chain | gacatcgtgctgactcagtcccctgcgactctgagcctgtcaccgggagaacgggccaccctctcttgccgcgcctcc |
| | | caatccattactcggaactacctggcctggtatcagcagaagccaggacaggcccctaggcttctgatctacggggcc |
| | | agctcaagagcaactggcatcccggctcgcttctccggttcgggaagcggcaccgacttcaccctgacaatttcgtccc |
| | | tcgaacccgaggatttcgccgtgtactactgccaacagcactccatgtacccccggacctttgggcagggaaccaaagt |
| | | cgagatcaagcgtacggtggccgctcccagcgtgttcatcttcccccccagcgacgagcagctgaagagcggcaccg |
| | | ccagcgtggtgtgcctgctgaacaacttctacccccgggaggccaaggtgcagtggaaggtggacaacgccctgcag |
| | | agcggcaacagccaggagagcgtcaccgagcaggacagcaaggactccacctacagcctgagcagcaccctgacc |
| | | ctgagcaaggccgactacgagaagcataaggtgtacgcctgcgaggtgacccaccagggcctgtccagccccgtga |
| | | ccaagagcttcaacaggggcgagtgc |
| XX09_LALA | | |
| SEQ ID NO: 28 | HCDR1 (Combined) | GFTFSSYWMN |
| SEQ ID NO: 29 | HCDR2 (Combined) | AISSDGSYTYYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 29 | HCDR2 (Kabat) | AISSDGSYTYYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 33 | HCDR2 (Chothia) | SSDGSY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 35 | HCDR2 (IMGT) | ISSDGSYT |
| SEQ ID NO: 36 | HCDR3 (IMGT) | ARDRYSMIYSYGAGAFDY |
| SEQ ID NO: 37 | VH | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSS |
| SEQ ID NO: 38 | DNA VH | caggtgcaattgctggaaagcggcggtggcctggtgcagccgggtggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctca |
| SEQ ID NO: 39 | Heavy Chain | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 40 | DNA Heavy Chain | caggtgcaattgctggaaagcggcggtggcctggtgcagccgggtggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctcagcctccaccaagggtccatcggt |
| | | cttccccctggcaccctcctccaagagcacctctgggggcacagcggccctgggctgcctggtcaaggactacttccc |
| | | cgaaccggtgacggtgtcgtggaactcaggcgccctgaccagcggcgtgcacaccttcccggctgtcctacagtcctc |
| | | aggactctactccctcagcagcgtggtgaccgtgccctccagcagcttgggcacccagacctacatctgcaacgtgaat |
| | | cacaagcccagcaacaccaaggtggacaagagagttgagcccaaatcttgtgacaaaactcacacatgcccaccgtg |
| | | cccagcacctgaagcagcggggggaccgtcagtcttcctcttccccccaaaacccaaggacaccctcatgatctcccg |
| | | gacccctgaggtcacatgcgtggtggtggacgtgagccacgaagaccctgaggtcaagttcaactggtacgtggacg |
| | | gcgtggaggtgcataatgccaagacaaagccgcgggaggagcagtacaacagcacgtaccgggtggtcagcgtcct |
| | | caccgtcctgcaccaggactggctgaatggcaaggagtacaagtgcaaggtctccaacaaagccctcccagccccca |
| | | tcgagaaaaccatctccaaagccaaagggcagccccgagaaccacaggtgtacaccctgcccccatcccgggagga |
| | | gatgaccaagaaccaggtcagcctgacctgcctggtcaaaggcttctatcccagcgacatcgccgtggagtgggaga |
| | | gcaatgggcagccggagaacaactacaagaccacgcctcccgtgctggactccgacggctccttcttcctctacagca |
| | | agctcaccgtggacaagagcaggtggcagcaggggaacgtcttctcatgctccgtgatgcatgaggctctgcacaacc |
| | | actacacgcagaagagcctctccctgtctccgggtaaa |
| SEQ ID NO: 41 | LCDR1 (Combined) | RASQGISSYLA |
| SEQ ID NO: 42 | LCDR2 (Combined) | TASTLQS |
| SEQ ID NO: 172 | LCDR3 (Combined) | QQEWAKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 172 | LCDR3 (Kabat) | QQEWAKPRT |
| SEQ ID NO: 44 | LCDR1 (Chothia) | SQGISSY |
| SEQ ID NO: 45 | LCDR2 (Chothia) | TAS |
| SEQ ID NO: 173 | LCDR3 (Chothia) | EWAKPR |
| SEQ ID NO: 47 | LCDR1 (IMGT) | QGISSY |
| SEQ ID NO: 45 | LCDR2 (IMGT) | TAS |
| SEQ ID NO: 172 | LCDR3 (IMGT) | QQEWAKPRT |
| SEQ ID NO: 174 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWAKPRTFGQGTKV |
| | | EIK |
| SEQ ID NO: 175 | DNA VL | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcaggaatgggctaaaccgcgtacctttggccagggcacgaaagttgaa |
| | | attaaa |
| SEQ ID NO: 176 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWAKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 177 | DNA Light Chain | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcaggaatgggctaaaccgcgtacctttggccagggcacgaaagttgaa |
| | | attaaacgtacggtggccgctcccagcgtgttcatcttcccccccagcgacgagcagctgaagagcggcaccgccag |
| | | cgtggtgtgcctgctgaacaacttctacccccgggaggccaaggtgcagtggaaggtggacaacgccctgcagagc |
| | | ggcaacagccaggaaagcgtcaccgagcaggacagcaaggactccacctacagcctgagcagcaccctgaccctg |
| | | agcaaggccgactacgagaagcacaaggtgtacgcctgcgaggtgacccaccagggcctgtccagccccgtgacc |
| | | aagagcttcaaccggggcgagtgt |
| XX11_LALA | | |
| SEQ ID NO: 28 | HCDR1 (Combined) | GFTFSSYWMN |
| SEQ ID NO: 29 | HCDR2 (Combined) | AISSDGSYTYYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 29 | HCDR2 (Kabat) | AISSDGSYTYYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 33 | HCDR2 (Chothia) | SSDGSY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 35 | HCDR2 (IMGT) | ISSDGSYT |
| SEQ ID NO: 36 | HCDR3 (IMGT) | ARDRYSMIYSYGAGAFDY |
| SEQ ID NO: 37 | VH | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSS |
| SEQ ID NO: 38 | DNA VH | caggtgcaattgctggaaagcggcggtggcctggtgcagccgggggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctca |
| SEQ ID NO: 39 | Heavy Chain | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 40 | DNA Heavy Chain | caggtgcaattgctggaaagcggcggtggcctggtgcagccgggggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctcagcctccaccaagggtccatcggt |
| | | cttccccctggcaccctcctccaagagcacctctgggggcacagcggccctgggctgcctggtcaaggactacttccc |
| | | cgaaccggtgacggtgtcgtggaactcaggcgccctgaccagcggcgtgcacaccttcccggctgtcctacagtcctc |
| | | aggactctactccctcagcagcgtggtgaccgtgccctccagcagcttgggcacccagacctacatctgcaacgtgaat |
| | | cacaagcccagcaacaccaaggtggacaagagagttgagcccaaatcttgtgacaaaactcacacatgcccaccgtg |
| | | cccagcacctgaagcagcggggggaccgtcagtcttcctcttccccccaaaacccaaggacaccctcatgatctcccg |
| | | gacccctgaggtcacatgcgtggtggtggacgtgagccacgaagaccctgaggtcaagttcaactggtacgtggacg |
| | | gcgtggaggtgcataatgccaagacaaagccgcgggaggagcagtacaacagcacgtaccgggtggtcagcgtcct |
| | | caccgtcctgcaccaggactggctgaatggcaaggagtacaagtgcaaggtctccaacaaagccctcccagccccca |
| | | tcgagaaaaccatctccaaagccaaagggcagccccgagaaccacaggtgtacaccctgcccccatcccgggagga |
| | | gatgaccaagaaccaggtcagcctgacctgcctggtcaaaggcttctatcccagcgacatcgccgtggagtgggaga |
| | | gcaatgggcagccggagaacaactacaagaccacgcctcccgtgctggactccgacggctccttcttcctctacagca |
| | | agctcaccgtggacaagagcaggtggcagcaggggaacgtcttctcatgctccgtgatgcatgaggctctgcacaacc |
| | | actacacgcagaagagcctctccctgtctccgggtaaa |
| SEQ ID NO: 41 | LCDR1 (Combined) | RASQGISSYLA |
| SEQ ID NO: 42 | LCDR2 (Combined) | TASTLQS |
| SEQ ID NO: 178 | LCDR3 (Combined) | QQSWTRPRT |
| SEQ ID NO: 41 | LCDR1 (Kabat) | RASQGISSYLA |
| SEQ ID NO: 42 | LCDR2 (Kabat) | TASTLQS |
| SEQ ID NO: 178 | LCDR3 (Kabat) | QQSWTRPRT |
| SEQ ID NO: 44 | LCDR1 (Chothia) | SQGISSY |
| SEQ ID NO: 45 | LCDR2 (Chothia) | TAS |
| SEQ ID NO: 179 | LCDR3 (Chothia) | SWTRPR |
| SEQ ID NO: 47 | LCDR1 (IMGT) | QGISSY |
| SEQ ID NO: 45 | LCDR2 (IMGT) | TAS |
| SEQ ID NO: 178 | LCDR3 (IMGT) | QQSWTRPRT |
| SEQ ID NO: 180 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQSWTRPRTFGQGTKV |
| | | EIK |
| SEQ ID NO: 181 | DNA VL | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcagtcttggactcgtccgcgtacctttggccagggcacgaaagttgaaa |
| | | ttaaa |
| SEQ ID NO: 182 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQSWTRPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 183 | DNA Light Chain | Gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcagtcttggactcgtccgcgtacctttggccagggcacgaaagttgaaa |
| | | ttaaacgtacggtggccgctcccagcgtgttcatcttcccccccagcgacgagcagctgaagagcggcaccgccagc |
| | | gtggtgtgcctgctgaacaacttctacccccgggaggccaaggtgcagtggaaggtggacaacgccctgcagagcg |
| | | gcaacagccaggaaagcgtcaccgagcaggacagcaaggactccacctacagcctgagcagcaccctgaccctga |
| | | gcaaggccgactacgagaagcacaaggtgtacgcctgcgaggtgacccaccagggcctgtccagccccgtgacca |
| | | agagcttcaaccggggcgagtgt |
| XX12_LALA | | |
| SEQ ID NO: 28 | HCDR1 (Combined) | GFTFSSYWMN |
| SEQ ID NO: 29 | HCDR2 (Combined) | AISSDGSYTYYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 29 | HCDR2 (Kabat) | AISSDGSYTYYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 33 | HCDR2 (Chothia) | SSDGSY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 35 | HCDR2 (IMGT) | ISSDGSYT |
| SEQ ID NO: 36 | HCDR3 (IMGT) | ARDRYSMIYSYGAGAFDY |
| SEQ ID NO: 37 | VH | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSS |
| SEQ ID NO: 38 | DNA VH | caggtgcaattgctggaaagcggcggtggcctggtgcagccgggggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctca |
| SEQ ID NO: 39 | Heavy Chain | QVQLLESGGGLVQPGGSLRLSCAASGFTFSSYWMNWVRQAPGKGLEWVS |
| | | AISSDGSYTYYADSVKGRFTISRDNSKNTLYLQMNSLRAEDTAVYYCARD |
| | | RYSMIYSYGAGAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 40 | DNA Heavy Chain | caggtgcaattgctggaaagcggcggtggcctggtgcagccgggggcagcctgcgtctgagctgcgcggcgtccg |
| | | gattcaccttttcttcttactggatgaactgggtgcgccaggccccgggcaaaggtctcgagtgggtttccgctatctcttc |
| | | tgacggttcttacacctactatgcggatagcgtgaaaggccgctttaccatcagccgcgataattcgaaaaacaccctgt |
| | | atctgcaaatgaacagcctgcgtgcggaagatacggccgtgtattattgcgcgcgtgaccgttactctatgatctactctt |
| | | acggtgctggtgctttcgattactggggccaaggcaccctggtgactgttagctcagcctccaccaagggtccatcggt |
| | | cttccccctggcaccctcctccaagagcacctctgggggcacagcggccctgggctgcctggtcaaggactacttccc |
| | | cgaaccggtgacggtgtcgtggaactcaggcgccctgaccagcggcgtgcacaccttcccggctgtcctacagtcctc |
| | | aggactctactccctcagcagcgtggtgaccgtgccctccagcagcttgggcacccagacctacatctgcaacgtgaat |
| | | cacaagcccagcaacaccaaggtggacaagagagttgagcccaaatcttgtgacaaaactcacacatgcccaccgtg |
| | | cccagcacctgaagcagcggggggaccgtcagtcttcctcttccccccaaaacccaaggacaccctcatgatctcccg |
| | | gacccctgaggtcacatgcgtggtggtggacgtgagccacgaagaccctgaggtcaagttcaactggtacgtggacg |
| | | gcgtggaggtgcataatgccaagacaaagccgcgggaggagcagtacaacagcacgtaccgggtggtcagcgtcct |
| | | caccgtcctgcaccaggactggctgaatggcaaggagtacaagtgcaaggtctccaacaaagccctcccagccccca |
| | | tcgagaaaaccatctccaaagccaaagggcagccccgagaaccacaggtgtacaccctgcccccatcccgggagga |
| | | gatgaccaagaaccaggtcagcctgacctgcctggtcaaaggcttctatcccagcgacatcgccgtggagtgggaga |
| | | gcaatgggcagccggagaacaactacaagaccacgcctcccgtgctggactccgacggctccttcttcctctacagca |
| | | agctcaccgtggacaagagcaggtggcagcaggggaacgtcttctcatgctccgtgatgcatgaggctctgcacaacc |
| | | actacacgcagaagagcctctccctgtctccgggtaaa |
| SEQ ID NO: 41 | LCDR1 (Combined) | RASQGISSYLA |
| SEQ ID NO: 42 | LCDR2 (Combined) | TASTLQS |
| SEQ ID NO: 184 | LCDR3 (Combined) | QQIWMAPRT |
| SEQ ID NO: 41 | LCDR1 (Kabat) | RASQGISSYLA |
| SEQ ID NO: 42 | LCDR2 (Kabat) | TASTLQS |
| SEQ ID NO: 184 | LCDR3 (Kabat) | QQIWMAPRT |
| SEQ ID NO: 44 | LCDR1 (Chothia) | SQGISSY |
| SEQ ID NO: 45 | LCDR2 (Chothia) | TAS |
| SEQ ID NO: 185 | LCDR3 (Chothia) | IWMAPR |
| SEQ ID NO: 47 | LCDR1 (IMGT) | QGISSY |
| SEQ ID NO: 45 | LCDR2 (IMGT) | TAS |
| SEQ ID NO: 184 | LCDR3 (IMGT) | QQIWMAPRT |
| SEQ ID NO: 186 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWMAPRTFGQGTKV |
| | | EIK |
| SEQ ID NO: 187 | DNA VL | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcagatctggatggctccgcgtacctttggccagggcacgaaagttgaa |
| | | attaaa |
| SEQ ID NO: 188 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWMAPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 189 | DNA Light Chain | gatatccagatgacccagagcccgagcagcctgagcgccagcgtgggcgatcgcgtgaccattacctgcagagcca |
| | | gccagggtatttcttcttacctggcttggtaccagcagaaaccgggcaaagcgccgaaactattaatctacactgcttcta |
| | | ctctgcaaagcggcgtgccgagccgctttagcggcagcggatccggcaccgatttcaccctgaccattagctctctgca |
| | | accggaagactttgcgacctattattgccagcagatctggatggctccgcgtacctttggccagggcacgaaagttgaa |
| | | attaaacgtacggtggccgctcccagcgtgttcatcttcccccccagcgacgagcagctgaagagcggcaccgccag |
| | | cgtggtgtgcctgctgaacaacttctacccccgggaggccaaggtgcagtggaaggtggacaacgccctgcagagc |
| | | ggcaacagccaggaaagcgtcaccgagcaggacagcaaggactccacctacagcctgagcagcaccctgaccctg |
| | | agcaaggccgactacgagaagcacaaggtgtacgcctgcgaggtgacccaccagggcctgtccagccccgtgacc |
| | | aagagcttcaaccggggcgagtgt |
| XX13_LALA | | |
| SEQ ID NO: 28 | HCDR1 (Combined) | GFTFSSYWMN |
| SEQ ID NO: 190 | HCDR2 (Combined) | AISSKGSYTYYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 190 | HCDR2 (Kabat) | AISSKGSYTYYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 191 | HCDR2 (Chothia) | SSKGSY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 192 | HCDR2 (IMGT) | ISSKGSYT |
| SEQ ID NO: 36 | HCDR3 (IMGT) | ARDRYSMIYSYGAGAFDY |
| SEQ ID NO: 193 | VH | QVQLLESGGGLVQPGGSLRLSCAASGFTFSSYWMNWVRQAPGKGLEWVS |
| | | AISSKGSYTYYADSVKGRFTISRDNSKNTLYLQMNSLRAEDTAVYYCARD |
| | | RYSMIYSYGAGAFDYWGQGTLVTVSS |
| SEQ ID NO: 194 | DNA VH | caagttcagctccttgagtctggggggggcctggtgcaacctgggggctctctgcggctttcatgtgcggcctcagggt |
| | | tcactttcagctcatactggatgaattgggtacgccaagctccaggcaaaggactcgaatgggtaagcgctatatccagc |
| | | aaagggagctatacctattacgcggattccgttaagggcaggttcactatatcccgcgacaactccaaaaatactttgtat |
| | | ctgcaaatgaattccctccgagccgaagataccgcagtatattactgtgcgagggacaggtactccatgatttacagcta |
| | | cggtgccggtgctttcgattattggggacaggggacacttgtgaccgtcagttct |
| SEQ ID NO: 195 | Heavy Chain | QVQLLESGGGLVQPGGSLRLSCAASGFTFSSYWMNWVRQAPGKGLEWVS |
| | | AISSKGSYTYYADSVKGRFTISRDNSKNTLYLQMNSLRAEDTAVYYCARD |
| | | RYSMIYSYGAGAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 196 | DNA Heavy Chain | caagttcagctccttgagtctggggggggcctggtgcaacctgggggctctctgcggctttcatgtgcggcctcagggt |
| | | tcactttcagctcatactggatgaattgggtacgccaagctccaggcaaaggactcgaatgggtaagcgctatatccagc |
| | | aaagggagctatacctattacgcggattccgttaagggcaggttcactatatcccgcgacaactccaaaaatactttgtat |
| | | ctgcaaatgaattccctccgagccgaagataccgcagtatattactgtgcgagggacaggtactccatgatttacagcta |
| | | cggtgccggtgctttcgattattggggacaggggacacttgtgaccgtcagttctgcaagtaccaaagggccgtctgtttt |
| | | cccattggctccctcatccaagagcacgagtggaggcaccgccgcgctgggatgccttgtgaaagactatttcccgga |
| | | gcccgtgaccgttagctggaacagcggcgctcttaccagtggcgttcacacattcccagctgttttgcagtcatccgggc |
| | | tctactctctctcatccgtggtcaccgtgccgtctagttctttgggcacccagacctacatctgtaacgtaaatcacaaacct |
| | | agtaatactaaggtggacaagcgagttgaaccgaagagctgtgataagacacatacttgtccaccatgtccggcaccc |
| | | gaggcagcgggggggcccagtgtttttctcttcccacccaagcccaaagacacattgatgatctcacgaaccccagag |
| | | gtaacttgtgtcgtggtagatgtaagccatgaggaccccgaagttaagttcaattggtatgttgacggtgtagaggtgcac |
| | | aatgccaaaactaaaccccgggaggagcaatacaactcaacttacagagtcgtatccgtgctgaccgttttgcaccagg |
| | | attggttgaatggtaaggaatacaaatgtaaagtgagcaataaagctctcccagcgcccatcgagaagaccattagcaa |
| | | agccaagggtcaacccagggaaccccaggtatatacgctgccaccctcaagggaagagatgacaaagaatcaagtgt |
| | | cactgacgtgtcttgtcaagggtttctatcctagcgacattgcggtggaatgggagtcaaatgggcaacccgagaacaa |
| | | ctacaagactactcctcccgtcctggacagcgacggctccttcttcctgtatagtaaactgaccgtcgataaaagtaggtg |
| | | gcagcaggggaatgtctttagttgctctgtcatgcatgaggcgctccataaccactacacccaaaaatctttgagcttgag |
| | | ccctgggaaa |
| SEQ ID NO: 41 | LCDR1 (Combined) | RASQGISSYLA |
| SEQ ID NO: 42 | LCDR2 (Combined) | TASTLQS |
| SEQ ID NO: 134 | LCDR3 (Combined) | QQTWRKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 134 | LCDR3 (Kabat) | QQTWRKPRT |
| SEQ ID NO: 44 | LCDR1 (Chothia) | SQGISSY |
| SEQ ID NO: 45 | LCDR2 (Chothia) | TAS |
| SEQ ID NO: 135 | LCDR3 (Chothia) | TWRKPR |
| SEQ ID NO: 47 | LCDR1 (IMGT) | QGISSY |
| SEQ ID NO: 45 | LCDR2 (IMGT) | TAS |
| SEQ ID NO: 134 | LCDR3 (IMGT) | QQTWRKPRT |
| SEQ ID NO: 136 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIK |
| SEQ ID NO: 197 | DNA VL | gacattcaaatgacacaaagtccgtccagtcttagtgcttctgtgggcgatagggtcaccatcacttgtcgggcgtctcag |
| | | gggatcagctcttacttggcatggtatcaacaaaagccaggaaaagcacctaaattgcttatttatacagcgtccaccctc |
| | | cagtcaggagtgcctagtaggttctcaggctctgggtccggtactgacttcacgctgactatatcaagcttgcaacccga |
| | | agattttgcaacatactactgccaacagacatggaggaagccaagaactttcggtcagggaacgaaagttgagataaa |
| | | g |
| SEQ ID NO: 138 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 198 | DNA Light Chain | gacattcaaatgacacaaagtccgtccagtcttagtgcttctgtgggcgatagggtcaccatcacttgtcgggcgtctcag |
| | | gggatcagctcttacttggcatggtatcaacaaaagccaggaaaagcacctaaattgcttatttatacagcgtccaccctc |
| | | cagtcaggagtgcctagtaggttctcaggctctgggtccggtactgacttcacgctgactatatcaagcttgcaacccga |
| | | agattttgcaacatactactgccaacagacatggaggaagccaagaactttcggtcagggaacgaaagttgagataaa |
| | | gcgcactgtcgcagcaccttccgtgttcattttcccgccttccgacgagcagcttaaatcagggaccgcgagtgttgtttg |
| | | cttgcttaataacttttacccacgggaagccaaagttcagtggaaggtggacaatgcactccaaagcgggaatagtcag |
| | | gagtcagttactgagcaagatagtaaagactctacttactctttgagttcaaccttgaccctctcaaaagcggactacgag |
| | | aagcataaagtgtacgcctgcgaggtgacgcatcaaggtttgtcttccccggttacgaagtcctttaataggggggaatg |
| | | t |
| XX14_LALA | | |
| SEQ ID NO: 28 | HCDR1 (Combined) | GFTFSSYWMN |
| SEQ ID NO: 190 | HCDR2 (Combined) | AISSKGSYTYYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 190 | HCDR2 (Kabat) | AISSKGSYTYYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 191 | HCDR2 (Chothia) | SSKGSY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 192 | HCDR2 (IMGT) | ISSKGSYT |
| SEQ ID NO: 36 | HCDR3 (IMGT) | ARDRYSMIYSYGAGAFDY |
| SEQ ID NO: 193 | VH | QVQLLESGGGLVQPGGSLRLSCAASGFTFSSYWMNWVRQAPGKGLEWVS |
| | | AISSKGSYTYYADSVKGRFTISRDNSKNTLYLQMNSLRAEDTAVYYCARD |
| | | RYSMIYSYGAGAFDYWGQGTLVTVSS |
| SEQ ID NO: 194 | DNA VH | caagttcagctccttgagtctggggggggcctggtgcaacctgggggctctctgcggctttcatgtgcggcctcagggt |
| | | tcactttcagctcatactggatgaattgggtacgccaagctccaggcaaaggactcgaatgggtaagcgctatatccagc |
| | | aaagggagctatacctattacgcggattccgttaagggcaggttcactatatcccgcgacaactccaaaaatactttgtat |
| | | ctgcaaatgaattccctccgagccgaagataccgcagtatattactgtgcgagggacaggtactccatgatttacagcta |
| | | cggtgccggtgctttcgattattggggacaggggacacttgtgaccgtcagttct |
| SEQ ID NO: 195 | Heavy Chain | QVQLLESGGGLVQPGGSLRLSCAASGFTFSSYWMNWVRQAPGKGLEWVS |
| | | AISSKGSYTYYADSVKGRFTISRDNSKNTLYLQMNSLRAEDTAVYYCARD |
| | | RYSMIYSYGAGAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 196 | DNA Heavy Chain | caagttcagctccttgagtctggggggggcctggtgcaacctgggggctctctgcggctttcatgtgcggcctcagggt |
| | | tcactttcagctcatactggatgaattgggtacgccaagctccaggcaaaggactcgaatgggtaagcgctatatccagc |
| | | aaagggagctatacctattacgcggattccgttaagggcaggttcactatatcccgcgacaactccaaaaatactttgtat |
| | | ctgcaaatgaattccctccgagccgaagataccgcagtatattactgtgcgagggacaggtactccatgatttacagcta |
| | | cggtgccggtgctttcgattattggggacaggggacacttgtgaccgtcagttctgcaagtaccaaagggccgtctgtttt |
| | | cccattggctccctcatccaagagcacgagtggaggcaccgccgcgctgggatgccttgtgaaagactatttcccgga |
| | | gcccgtgaccgttagctggaacagcggcgctcttaccagtggcgttcacacattcccagctgttttgcagtcatccgggc |
| | | tctactctctctcatccgtggtcaccgtgccgtctagttctttgggcacccagacctacatctgtaacgtaaatcacaaacct |
| | | agtaatactaaggtggacaagcgagttgaaccgaagagctgtgataagacacatacttgtccaccatgtccggcaccc |
| | | gaggcagcgggggggcccagtgtttttctcttcccacccaagcccaaagacacattgatgatctcacgaaccccagag |
| | | gtaacttgtgtcgtggtagatgtaagccatgaggaccccgaagttaagttcaattggtatgttgacggtgtagaggtgcac |
| | | aatgccaaaactaaaccccgggaggagcaatacaactcaacttacagagtcgtatccgtgctgaccgttttgcaccagg |
| | | attggttgaatggtaaggaatacaaatgtaaagtgagcaataaagctctcccagcgcccatcgagaagaccattagcaa |
| | | agccaagggtcaacccagggaaccccaggtatatacgctgccaccctcaagggaagagatgacaaagaatcaagtgt |
| | | cactgacgtgtcttgtcaagggtttctatcctagcgacattgcggtggaatgggagtcaaatgggcaacccgagaacaa |
| | | ctacaagactactcctcccgtcctggacagcgacggctccttcttcctgtatagtaaactgaccgtcgataaaagtaggtg |
| | | gcagcaggggaatgtctttagttgctctgtcatgcatgaggcgctccataaccactacacccaaaaatctttgagcttgag |
| | | ccctgggaaa |
| SEQ ID NO: 41 | LCDR1 (Combined) | RASQGISSYLA |
| SEQ ID NO: 42 | LCDR2 (Combined) | TASTLQS |
| SEQ ID NO: 172 | LCDR3 (Combined) | QQEWAKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 172 | LCDR3 (Kabat) | QQEWAKPRT |
| SEQ ID NO: 44 | LCDR1 (Chothia) | SQGISSY |
| SEQ ID NO: 45 | LCDR2 (Chothia) | TAS |
| SEQ ID NO: 173 | LCDR3 (Chothia) | EWAKPR |
| SEQ ID NO: 47 | LCDR1 (IMGT) | QGISSY |
| SEQ ID NO: 45 | LCDR2 (IMGT) | TAS |
| SEQ ID NO: 172 | LCDR3 (IMGT) | QQEWAKPRT |
| SEQ ID NO: 174 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWAKPRTFGQGTKV |
| | | EIK |
| SEQ ID NO: 199 | DNA VL | gatatacagatgacgcaaagtccctctagtctttctgcaagtgtcggggacagagttaccattacctgcagagcgtcaca |
| | | aggcatctctagttatctcgcgtggtaccaacagaagccaggtaaagcacctaaactgttgatttacacggcatcaacatt |
| | | gcagtcaggtgtcccctcccgatttagtggcagtggtagcggtacagattttactcttaccatttcatctcttcagccagaa |
| | | gattttgctacgtactactgtcaacaagaatgggctaaaccacgaacctttggacagggtacgaaggtcgaaataaaa |
| SEQ ID NO: 176 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWAKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 200 | DNA Light Chain | gatatacagatgacgcaaagtccctctagtctttctgcaagtgtcggggacagagttaccattacctgcagagcgtcaca |
| | | aggcatctctagttatctcgcgtggtaccaacagaagccaggtaaagcacctaaactgttgatttacacggcatcaacatt |
| | | gcagtcaggtgtcccctcccgatttagtggcagtggtagcggtacagattttactcttaccatttcatctcttcagccagaa |
| | | gattttgctacgtactactgtcaacaagaatgggctaaaccacgaacctttggacagggtacgaaggtcgaaataaaac |
| | | ggaccgttgccgccccctccgtcttcatcttccccccgtctgacgagcagctcaaatccggcacagcttctgtagtctgct |
| | | tgctgaataacttctacccaagagaagccaaagttcagtggaaggtcgataatgcattgcaatctggtaatagtcaggaa |
| | | tctgtgactgagcaggatagcaaagactcaacttacagcctctcttcaaccttgacgttgtccaaagcggattatgagaaa |
| | | cacaaggtgtacgcttgcgaggtgacgcatcaagggcttagttccccggtaaccaaatctttcaaccgaggtgaatgc |
| XX15_LALA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 201 | VH | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 202 | DNA VH | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagc |
| SEQ ID NO: 203 | Heavy Chain | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 204 | DNA Heavy Chain | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagcgcctcaacgaaaggaccgtccgtgttt |
| | | cctcttgctcctagctccaaatccacctcaggtggaacggccgccctggggtgcctggtaaaggactatttcccagagc |
| | | cagttactgtgtcttggaattctggtgcattgacaagtggcgtacacacttttcccgcggtcctccaatctagtggtctgtac |
| | | tcactgtcctccgttgtgactgtcccaagtagctcacttggcacacagacttacatctgtaatgttaatcataagccgtcaaa |
| | | cacgaaggtggataagagggtagaacctaagtcatgtgacaaaacgcatacttgccccccctgccctgcgccggaag |
| | | ccgctggcggaccctccgtattcttgttccctccaaagccaaaggacactctgatgattagccggacaccggaggtcac |
| | | ttgtgttgtagttgacgtcagccatgaggatcctgaggtgaaatttaattggtacgtggacggggttgaagtccacaatgc |
| | | taaaactaaacctagggaagagcaatataatagtacatacagggttgtcagtgtgctgaccgttctccatcaggactggc |
| | | tgaacggcaaggaatacaagtgcaaggtcagcaacaaggccttgccggcccccatcgagaagacgatctccaaagc |
| | | caaggggcaaccccgagaaccgcaggtatacacgctcccccctagtagagaagagatgacaaagaatcaagtttcctt |
| | | gacgtgccttgtgaaaggcttctaccctagtgacatcgcagtcgaatgggagagcaacgggcagccggagaataacta |
| | | taaaacaaccccccccgtgcttgactcagacgggtcattttttctgtatagcaaattgactgttgataaatcacggtggcaa |
| | | caaggaaacgtgtttagttgcagcgtaatgcacgaagctctccacaatcactatactcaaaagtcactgtcactctcccct |
| | | ggcaag |
| SEQ ID NO: 41 | LCDR1 (Combined) | RASQGISSYLA |
| SEQ ID NO: 42 | LCDR2 (Combined) | TASTLQS |
| SEQ ID NO: 126 | LCDR3 (Combined) | QQEWVKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 126 | LCDR3 (Kabat) | QQEWVKPRT |
| SEQ ID NO: 44 | LCDR1 (Chothia) | SQGISSY |
| SEQ ID NO: 45 | LCDR2 (Chothia) | TAS |
| SEQ ID NO: 127 | LCDR3 (Chothia) | EWVKPR |
| SEQ ID NO: 47 | LCDR1 (IMGT) | QGISSY |
| SEQ ID NO: 45 | LCDR2 (IMGT) | TAS |
| SEQ ID NO: 126 | LCDR3 (IMGT) | QQEWVKPRT |
| SEQ ID NO: 128 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWVKPRTFGQGTKV |
| | | EIK |
| SEQ ID NO: 205 | DNA VL | gacatacaaatgacgcaatctccgagtagcttgtcagcgtccgtaggcgaccgagtaacgattacgtgtagagcgagc |
| | | cagggaatttcatcttatttggcttggtatcagcaaaagccgggaaaagcacccaaactcctcatttatactgccagcacg |
| | | ttgcaaagcggcgttccgagtcggttctctggatcagggtccgggacggacttcaccttgacgatttcatctttgcaacct |
| | | gaagattttgcaacatactactgtcaacaggagtgggtgaagccaaggaccttcggacaaggcacgaaggtcgaaatc |
| | | aag |
| SEQ ID NO: 130 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWVKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 206 | DNA Light Chain | gacatacaaatgacgcaatctccgagtagcttgtcagcgtccgtaggcgaccgagtaacgattacgtgtagagcgagc |
| | | cagggaatttcatcttatttggcttggtatcagcaaaagccgggaaaagcacccaaactcctcatttatactgccagcacg |
| | | ttgcaaagcggcgttccgagtcggttctctggatcagggtccgggacggacttcaccttgacgatttcatctttgcaacct |
| | | gaagattttgcaacatactactgtcaacaggagtgggtgaagccaaggaccttcggacaaggcacgaaggtcgaaatc |
| | | aagcgaaccgtggcagctccgtccgtgtttatttttccgccttccgacgaacaacttaaaagtggaacagcctctgtcgtc |
| | | tgtctccttaacaacttctaccccagggaagctaaagtacagtggaaggtagataacgctctgcaaagtggtaattctcag |
| | | gagagcgtcacggaacaggactccaaagactccacctattctctgagctctacactgacgctcagcaaggcagactac |
| | | gaaaagcacaaagtatatgcgtgtgaggtgacgcatcaaggccttagcagtccagttacaaaaagttttaacaggggag |
| | | aatgc |
| XX15_DAPA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 122 | VH | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 207 | DNA VH | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctcc |
| SEQ ID NO: 208 | Heavy Chain | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPSVFLFPPKP |
| | | KDTLMISRTPEVTCVVVAVSHEDPEVKFNWYVDGVEVHNAKTKPREEQY |
| | | NSTYRVVSVLTVLHQDWLNGKEYKCKVSNKALAAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 209 | DNA Heavy Chain | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctccgcctcaaccaagggccc |
| | | gtcagtgttcccgctggctccatcgtcgaagtccacctccggaggaaccgcagcactcggttgcctggtcaaggactac |
| | | ttccctgagccagtgaccgtgtcgtggaacagcggagccctgacttccggcgtgcacacttttcccgcggtgctgcagt |
| | | cctccggtctgtactccctttcgtccgtggtcaccgtgccgtcgtctagcctgggcacccagacctacatctgcaacgtg |
| | | aaccacaagccgtccaacaccaaagtggataagcgggtggagccgaagtcctgcgataagacacacacgtgcccgc |
| | | catgtccagcgcctgaattgcttggcggaccttccgtgttcctgttcccgcctaagcccaaggacaccttgatgattagcc |
| | | ggactcccgaagtcacctgtgtggtggtggcagtgtcccacgaggaccccgaggtcaagtttaattggtacgtggacg |
| | | gcgtcgaagtgcacaacgccaagactaagccccgggaggaacagtacaacagcacctaccgggtcgtgtccgtgct |
| | | gaccgtgctgcaccaggactggctgaatgggaaagagtacaagtgcaaagtgtccaacaaggccttggccgctcctat |
| | | cgaaaaaactatcagcaaggctaagggacagccgagggaaccccaagtctacaccctgcccccttcacgcgaagag |
| | | atgaccaagaatcaagtgtcgctgacctgcctcgtcaagggattctacccctccgacattgcggtggagtgggagtcca |
| | | acggccagcccgagaacaactacaagactactccgcccgtgctggactccgacggcagcttcttcctgtattccaagct |
| | | gaccgtggacaagtcccggtggcagcaaggaaacgtgttctcctgctcggtcatgcacgaagccctgcacaaccacta |
| | | tacgcagaagtccctgtccttgagcccggggaaa |
| SEQ ID NO: 41 | LCDR1 (Combined) | RASQGISSYLA |
| SEQ ID NO: 42 | LCDR2 (Combined) | TASTLQS |
| SEQ ID NO: 126 | LCDR3 (Combined) | QQEWVKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 126 | LCDR3 (Kabat) | QQEWVKPRT |
| SEQ ID NO: 44 | LCDR1 (Chothia) | SQGISSY |
| SEQ ID NO: 45 | LCDR2 (Chothia) | TAS |
| SEQ ID NO: 127 | LCDR3 (Chothia) | EWVKPR |
| SEQ ID NO: 47 | LCDR1 (IMGT) | QGISSY |
| SEQ ID NO: 45 | LCDR2 (IMGT) | TAS |
| SEQ ID NO: 126 | LCDR3 (IMGT) | QQEWVKPRT |
| SEQ ID NO: 128 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWVKPRTFGQGTKV |
| | | EIK |
| SEQ ID NO: 210 | DNA VL | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaagaatgggtgaagcccaggacatttggccagggcactaaggtcga |
| | | gattaag |
| SEQ ID NO: 130 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWVKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 211 | DNA Light Chain | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaagaatgggtgaagcccaggacatttggccagggcactaaggtcga |
| | | gattaagcgtacggtggccgctcccagcgtgttcatcttcccccccagcgacgagcagctgaagagcggcaccgcca |
| | | gcgtggtgtgcctgctgaacaacttctacccccgggaggccaaggtgcagtggaaggtggacaacgccctgcagag |
| | | cggcaacagccaggagagcgtcaccgagcaggacagcaaggactccacctacagcctgagcagcaccctgaccct |
| | | gagcaaggccgactacgagaagcataaggtgtacgcctgcgaggtgacccaccagggcctgtccagccccgtgacc |
| | | aagagcttcaacaggggcgagtgc |
| XX16_LALA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 201 | VH | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 202 | DNA VH | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagc |
| SEQ ID NO: 203 | Heavy Chain | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 204 | DNA Heavy Chain | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagcgcctcaacgaaaggaccgtccgtgttt |
| | | cctcttgctcctagctccaaatccacctcaggtggaacggccgccctggggtgcctggtaaaggactatttcccagagc |
| | | cagttactgtgtcttggaattctggtgcattgacaagtggcgtacacacttttcccgcggtcctccaatctagtggtctgtac |
| | | tcactgtcctccgttgtgactgtcccaagtagctcacttggcacacagacttacatctgtaatgttaatcataagccgtcaaa |
| | | cacgaaggtggataagagggtagaacctaagtcatgtgacaaaacgcatacttgccccccctgccctgcgccggaag |
| | | ccgctggcggaccctccgtattcttgttccctccaaagccaaaggacactctgatgattagccggacaccggaggtcac |
| | | ttgtgttgtagttgacgtcagccatgaggatcctgaggtgaaatttaattggtacgtggacggggttgaagtccacaatgc |
| | | taaaactaaacctagggaagagcaatataatagtacatacagggttgtcagtgtgctgaccgttctccatcaggactggc |
| | | tgaacggcaaggaatacaagtgcaaggtcagcaacaaggccttgccggcccccatcgagaagacgatctccaaagc |
| | | caaggggcaaccccgagaaccgcaggtatacacgctcccccctagtagagaagagatgacaaagaatcaagtttcctt |
| | | gacgtgccttgtgaaaggcttctaccctagtgacatcgcagtcgaatgggagagcaacgggcagccggagaataacta |
| | | taaaacaaccccccccgtgcttgactcagacgggtcattttttctgtatagcaaattgactgttgataaatcacggtggcaa |
| | | caaggaaacgtgtttagttgcagcgtaatgcacgaagctctccacaatcactatactcaaaagtcactgtcactctcccct |
| | | ggcaag |
| SEQ ID NO: 41 | LCDR1 (Combined) | RASQGISSYLA |
| SEQ ID NO: 42 | LCDR2 (Combined) | TASTLQS |
| SEQ ID NO: 134 | LCDR3 (Combined) | QQTWRKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 134 | LCDR3 (Kabat) | QQTWRKPRT |
| SEQ ID NO: 44 | LCDR1 (Chothia) | SQGISSY |
| SEQ ID NO: 45 | LCDR2 (Chothia) | TAS |
| SEQ ID NO: 135 | LCDR3 (Chothia) | TWRKPR |
| SEQ ID NO: 47 | LCDR1 (IMGT) | QGISSY |
| SEQ ID NO: 45 | LCDR2 (IMGT) | TAS |
| SEQ ID NO: 134 | LCDR3 (IMGT) | QQTWRKPRT |
| SEQ ID NO: 136 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIK |
| SEQ ID NO: 197 | DNA VL | gacattcaaatgacacaaagtccgtccagtcttagtgcttctgtgggcgatagggtcaccatcacttgtcgggcgtctcag |
| | | gggatcagctcttacttggcatggtatcaacaaaagccaggaaaagcacctaaattgcttatttatacagcgtccaccctc |
| | | cagtcaggagtgcctagtaggttctcaggctctgggtccggtactgacttcacgctgactatatcaagcttgcaacccga |
| | | agattttgcaacatactactgccaacagacatggaggaagccaagaactttcggtcagggaacgaaagttgagataaa |
| | | g |
| SEQ ID NO: 138 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 198 | DNA Light Chain | gacattcaaatgacacaaagtccgtccagtcttagtgcttctgtgggcgatagggtcaccatcacttgtcgggcgtctcag |
| | | gggatcagctcttacttggcatggtatcaacaaaagccaggaaaagcacctaaattgcttatttatacagcgtccaccctc |
| | | cagtcaggagtgcctagtaggttctcaggctctgggtccggtactgacttcacgctgactatatcaagcttgcaacccga |
| | | agattttgcaacatactactgccaacagacatggaggaagccaagaactttcggtcagggaacgaaagttgagataaa |
| | | gcgcactgtcgcagcaccttccgtgttcattttcccgccttccgacgagcagcttaaatcagggaccgcgagtgttgtttg |
| | | cttgcttaataacttttacccacgggaagccaaagttcagtggaaggtggacaatgcactccaaagcgggaatagtcag |
| | | gagtcagttactgagcaagatagtaaagactctacttactctttgagttcaaccttgaccctctcaaaagcggactacgag |
| | | aagcataaagtgtacgcctgcgaggtgacgcatcaaggtttgtcttccccggttacgaagtcctttaataggggggaatg |
| | | t |
| XX16_DAPA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 122 | VH | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 207 | DNA VH | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctcc |
| SEQ ID NO: 208 | Heavy Chain | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPSVFLFPPKP |
| | | KDTLMISRTPEVTCVVVAVSHEDPEVKFNWYVDGVEVHNAKTKPREEQY |
| | | NSTYRVVSVLTVLHQDWLNGKEYKCKVSNKALAAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 209 | DNA Heavy Chain | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctccgcctcaaccaagggccc |
| | | gtcagtgttcccgctggctccatcgtcgaagtccacctccggaggaaccgcagcactcggttgcctggtcaaggactac |
| | | ttccctgagccagtgaccgtgtcgtggaacagcggagccctgacttccggcgtgcacacttttcccgcggtgctgcagt |
| | | cctccggtctgtactccctttcgtccgtggtcaccgtgccgtcgtctagcctgggcacccagacctacatctgcaacgtg |
| | | aaccacaagccgtccaacaccaaagtggataagcgggtggagccgaagtcctgcgataagacacacacgtgcccgc |
| | | catgtccagcgcctgaattgcttggcggaccttccgtgttcctgttcccgcctaagcccaaggacaccttgatgattagcc |
| | | ggactcccgaagtcacctgtgtggtggtggcagtgtcccacgaggaccccgaggtcaagtttaattggtacgtggacg |
| | | gcgtcgaagtgcacaacgccaagactaagccccgggaggaacagtacaacagcacctaccgggtcgtgtccgtgct |
| | | gaccgtgctgcaccaggactggctgaatgggaaagagtacaagtgcaaagtgtccaacaaggccttggccgctcctat |
| | | cgaaaaaactatcagcaaggctaagggacagccgagggaaccccaagtctacaccctgcccccttcacgcgaagag |
| | | atgaccaagaatcaagtgtcgctgacctgcctcgtcaagggattctacccctccgacattgcggtggagtgggagtcca |
| | | acggccagcccgagaacaactacaagactactccgcccgtgctggactccgacggcagcttcttcctgtattccaagct |
| | | gaccgtggacaagtcccggtggcagcaaggaaacgtgttctcctgctcggtcatgcacgaagccctgcacaaccacta |
| | | tacgcagaagtccctgtccttgagcccggggaaa |
| SEQ ID NO: 41 | LCDR1 (Combined) | RASQGISSYLA |
| SEQ ID NO: 42 | LCDR2 (Combined) | TASTLQS |
| SEQ ID NO: 134 | LCDR3 (Combined) | QQTWRKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 134 | LCDR3 (Kabat) | QQTWRKPRT |
| SEQ ID NO: 44 | LCDR1 (Chothia) | SQGISSY |
| SEQ ID NO: 45 | LCDR2 (Chothia) | TAS |
| SEQ ID NO: 135 | LCDR3 (Chothia) | TWRKPR |
| SEQ ID NO: 47 | LCDR1 (IMGT) | QGISSY |
| SEQ ID NO: 45 | LCDR2 (IMGT) | TAS |
| SEQ ID NO: 134 | LCDR3 (IMGT) | QQTWRKPRT |
| SEQ ID NO: 136 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIK |
| SEQ ID NO: 143 | DNA VL | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaacctggcggaagcccaggacatttggccagggcactaaggtcga |
| | | gattaag |
| SEQ ID NO: 138 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 144 | DNA Light Chain | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaacctggcggaagcccaggacatttggccagggcactaaggtcga |
| | | gattaagcgtacggtggccgctcccagcgtgttcatcttcccccccagcgacgagcagctgaagagcggcaccgcca |
| | | gcgtggtgtgcctgctgaacaacttctacccccgggaggccaaggtgcagtggaaggtggacaacgccctgcagag |
| | | cggcaacagccaggagagcgtcaccgagcaggacagcaaggactccacctacagcctgagcagcaccctgaccct |
| | | gagcaaggccgactacgagaagcataaggtgtacgcctgcgaggtgacccaccagggcctgtccagccccgtgacc |
| | | aagagcttcaacaggggcgagtgc |
| XX17_LALA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 201 | VH | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 202 | DNA VH | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagc |
| SEQ ID NO: 203 | Heavy Chain | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 204 | DNA Heavy Chain | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagcgcctcaacgaaaggaccgtccgtgttt |
| | | cctcttgctcctagctccaaatccacctcaggtggaacggccgccctggggtgcctggtaaaggactatttcccagagc |
| | | cagttactgtgtcttggaattctggtgcattgacaagtggcgtacacacttttcccgcggtcctccaatctagtggtctgtac |
| | | tcactgtcctccgttgtgactgtcccaagtagctcacttggcacacagacttacatctgtaatgttaatcataagccgtcaaa |
| | | cacgaaggtggataagagggtagaacctaagtcatgtgacaaaacgcatacttgccccccctgccctgcgccggaag |
| | | ccgctggcggaccctccgtattcttgttccctccaaagccaaaggacactctgatgattagccggacaccggaggtcac |
| | | ttgtgttgtagttgacgtcagccatgaggatcctgaggtgaaatttaattggtacgtggacggggttgaagtccacaatgc |
| | | taaaactaaacctagggaagagcaatataatagtacatacagggttgtcagtgtgctgaccgttctccatcaggactggc |
| | | tgaacggcaaggaatacaagtgcaaggtcagcaacaaggccttgccggcccccatcgagaagacgatctccaaagc |
| | | caaggggcaaccccgagaaccgcaggtatacacgctcccccctagtagagaagagatgacaaagaatcaagtttcctt |
| | | gacgtgccttgtgaaaggcttctaccctagtgacatcgcagtcgaatgggagagcaacgggcagccggagaataacta |
| | | taaaacaaccccccccgtgcttgactcagacgggtcattttttctgtatagcaaattgactgttgataaatcacggtggcaa |
| | | caaggaaacgtgtttagttgcagcgtaatgcacgaagctctccacaatcactatactcaaaagtcactgtcactctcccct |
| | | ggcaag |
| SEQ ID NO: 41 | LCDR1 (Combined) | RASQGISSYLA |
| SEQ ID NO: 42 | LCDR2 (Combined) | TASTLQS |
| SEQ ID NO: 145 | LCDR3 (Combined) | QQIWTVPRT |
| SEQ ID NO: 41 | LCDR1 (Kabat) | RASQGISSYLA |
| SEQ ID NO: 42 | LCDR2 (Kabat) | TASTLQS |
| SEQ ID NO: 145 | LCDR3 (Kabat) | QQIWTVPRT |
| SEQ ID NO: 44 | LCDR1 (Chothia) | SQGISSY |
| SEQ ID NO: 45 | LCDR2 (Chothia) | TAS |
| SEQ ID NO: 146 | LCDR3 (Chothia) | IWTVPR |
| SEQ ID NO: 47 | LCDR1 (IMGT) | QGISSY |
| SEQ ID NO: 45 | LCDR2 (IMGT) | TAS |
| SEQ ID NO: 145 | LCDR3 (IMGT) | QQIWTVPRT |
| SEQ ID NO: 147 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWTVPRTFGQGTKV |
| | | EIK |
| SEQ ID NO: 212 | DNA VL | gacatacagatgactcagagtccttcctccctcagtgcttcagtgggtgatcgcgtgacgatcacgtgcagagcctcaca |
| | | agggatctccagttacctggcctggtatcaacaaaaaccaggcaaggcgcctaagctgttgatatatacggcatctacat |
| | | tgcagtctggggtaccaagtcgattcagtggttctggctcaggcactgactttacccttacaatatcaagtcttcagccgg |
| | | aggatttcgcaacttactattgccagcagatttggacggtgccgcgcactttcggtcagggaacaaaggtggaaataaa |
| | | a |
| SEQ ID NO: 149 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWTVPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 213 | DNA Light Chain | gacatacagatgactcagagtccttcctccctcagtgcttcagtgggtgatcgcgtgacgatcacgtgcagagcctcaca |
| | | agggatctccagttacctggcctggtatcaacaaaaaccaggcaaggcgcctaagctgttgatatatacggcatctacat |
| | | tgcagtctggggtaccaagtcgattcagtggttctggctcaggcactgactttacccttacaatatcaagtcttcagccgg |
| | | aggatttcgcaacttactattgccagcagatttggacggtgccgcgcactttcggtcagggaacaaaggtggaaataaa |
| | | aagaacggtcgcagcaccgagtgttttcatcttccctccctccgacgagcagcttaaaagcggtacagccagcgtagtg |
| | | tgtttgttgaataatttttatccacgcgaagcaaaagttcagtggaaggtagacaacgcattgcaaagcggaaattcccaa |
| | | gaaagtgttacggagcaagacagtaaggactctacatattccttgtcatcaacactcacccttagtaaagcagattacga |
| | | gaaacacaaggtctatgcatgtgaggtaacgcatcagggcctctccagtcccgtcaccaagtccttcaacaggggtga |
| | | gtgc |
| XX17_DAPA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 122 | VH | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 207 | DNA VH | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctcc |
| SEQ ID NO: 208 | Heavy Chain | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPSVFLFPPKP |
| | | KDTLMISRTPEVTCVVVAVSHEDPEVKFNWYVDGVEVHNAKTKPREEQY |
| | | NSTYRVVSVLTVLHQDWLNGKEYKCKVSNKALAAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 209 | DNA Heavy Chain | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctccgcctcaaccaagggccc |
| | | gtcagtgttcccgctggctccatcgtcgaagtccacctccggaggaaccgcagcactcggttgcctggtcaaggactac |
| | | ttccctgagccagtgaccgtgtcgtggaacagcggagccctgacttccggcgtgcacacttttcccgcggtgctgcagt |
| | | cctccggtctgtactccctttcgtccgtggtcaccgtgccgtcgtctagcctgggcacccagacctacatctgcaacgtg |
| | | aaccacaagccgtccaacaccaaagtggataagcgggtggagccgaagtcctgcgataagacacacacgtgcccgc |
| | | catgtccagcgcctgaattgcttggcggaccttccgtgttcctgttcccgcctaagcccaaggacaccttgatgattagcc |
| | | ggactcccgaagtcacctgtgtggtggtggcagtgtcccacgaggaccccgaggtcaagtttaattggtacgtggacg |
| | | gcgtcgaagtgcacaacgccaagactaagccccgggaggaacagtacaacagcacctaccgggtcgtgtccgtgct |
| | | gaccgtgctgcaccaggactggctgaatgggaaagagtacaagtgcaaagtgtccaacaaggccttggccgctcctat |
| | | cgaaaaaactatcagcaaggctaagggacagccgagggaaccccaagtctacaccctgcccccttcacgcgaagag |
| | | atgaccaagaatcaagtgtcgctgacctgcctcgtcaagggattctacccctccgacattgcggtggagtgggagtcca |
| | | acggccagcccgagaacaactacaagactactccgcccgtgctggactccgacggcagcttcttcctgtattccaagct |
| | | gaccgtggacaagtcccggtggcagcaaggaaacgtgttctcctgctcggtcatgcacgaagccctgcacaaccacta |
| | | tacgcagaagtccctgtccttgagcccggggaaa |
| SEQ ID NO: 41 | LCDR1 (Combined) | RASQGISSYLA |
| SEQ ID NO: 42 | LCDR2 (Combined) | TASTLQS |
| SEQ ID NO: 145 | LCDR3 (Combined) | QQIWTVPRT |
| SEQ ID NO: 41 | LCDR1 (Kabat) | RASQGISSYLA |
| SEQ ID NO: 42 | LCDR2 (Kabat) | TASTLQS |
| SEQ ID NO: 145 | LCDR3 (Kabat) | QQIWTVPRT |
| SEQ ID NO: 44 | LCDR1 (Chothia) | SQGISSY |
| SEQ ID NO: 45 | LCDR2 (Chothia) | TAS |
| SEQ ID NO: 146 | LCDR3 (Chothia) | IWTVPR |
| SEQ ID NO: 47 | LCDR1 (IMGT) | QGISSY |
| SEQ ID NO: 45 | LCDR2 (IMGT) | TAS |
| SEQ ID NO: 145 | LCDR3 (IMGT) | QQIWTVPRT |
| SEQ ID NO: 147 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWTVPRTFGQGTKV |
| | | EIK |
| SEQ ID NO: 214 | DNA VL | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaatctggaccgtgcccaggacatttggccagggcactaaggtcgag |
| | | attaag |
| SEQ ID NO: 149 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWTVPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 215 | DNA Light Chain | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaatctggaccgtgcccaggacatttggccagggcactaaggtcgag |
| | | attaagcgtacggtggccgctcccagcgtgttcatcttcccccccagcgacgagcagctgaagagcggcaccgccag |
| | | cgtggtgtgcctgctgaacaacttctacccccgggaggccaaggtgcagtggaaggtggacaacgccctgcagagc |
| | | ggcaacagccaggagagcgtcaccgagcaggacagcaaggactccacctacagcctgagcagcaccctgaccctg |
| | | agcaaggccgactacgagaagcataaggtgtacgcctgcgaggtgacccaccagggcctgtccagccccgtgacca |
| | | agagcttcaacaggggcgagtgc |
| XX18_LALA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 201 | VH | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 202 | DNA VH | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagc |
| SEQ ID NO: 203 | Heavy Chain | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 204 | DNA Heavy Chain | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagcgcctcaacgaaaggaccgtccgtgttt |
| | | cctcttgctcctagctccaaatccacctcaggtggaacggccgccctggggtgcctggtaaaggactatttcccagagc |
| | | cagttactgtgtcttggaattctggtgcattgacaagtggcgtacacacttttcccgcggtcctccaatctagtggtctgtac |
| | | tcactgtcctccgttgtgactgtcccaagtagctcacttggcacacagacttacatctgtaatgttaatcataagccgtcaaa |
| | | cacgaaggtggataagagggtagaacctaagtcatgtgacaaaacgcatacttgccccccctgccctgcgccggaag |
| | | ccgctggcggaccctccgtattcttgttccctccaaagccaaaggacactctgatgattagccggacaccggaggtcac |
| | | ttgtgttgtagttgacgtcagccatgaggatcctgaggtgaaatttaattggtacgtggacggggttgaagtccacaatgc |
| | | taaaactaaacctagggaagagcaatataatagtacatacagggttgtcagtgtgctgaccgttctccatcaggactggc |
| | | tgaacggcaaggaatacaagtgcaaggtcagcaacaaggccttgccggcccccatcgagaagacgatctccaaagc |
| | | caaggggcaaccccgagaaccgcaggtatacacgctcccccctagtagagaagagatgacaaagaatcaagtttcctt |
| | | gacgtgccttgtgaaaggcttctaccctagtgacatcgcagtcgaatgggagagcaacgggcagccggagaataacta |
| | | taaaacaaccccccccgtgcttgactcagacgggtcattttttctgtatagcaaattgactgttgataaatcacggtggcaa |
| | | caaggaaacgtgtttagttgcagcgtaatgcacgaagctctccacaatcactatactcaaaagtcactgtcactctcccct |
| | | ggcaag |
| SEQ ID NO: 41 | LCDR1 (Combined) | RASQGISSYLA |
| SEQ ID NO: 42 | LCDR2 (Combined) | TASTLQS |
| SEQ ID NO: 172 | LCDR3 (Combined) | QQEWAKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 172 | LCDR3 (Kabat) | QQEWAKPRT |
| SEQ ID NO: 44 | LCDR1 (Chothia) | SQGISSY |
| SEQ ID NO: 45 | LCDR2 (Chothia) | TAS |
| SEQ ID NO: 173 | LCDR3 (Chothia) | EWAKPR |
| SEQ ID NO: 47 | LCDR1 (IMGT) | QGISSY |
| SEQ ID NO: 45 | LCDR2 (IMGT) | TAS |
| SEQ ID NO: 172 | LCDR3 (IMGT) | QQEWAKPRT |
| SEQ ID NO: 174 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWAKPRTFGQGTKV |
| | | EIK |
| SEQ ID NO: 199 | DNA VL | gatatacagatgacgcaaagtccctctagtctttctgcaagtgtcggggacagagttaccattacctgcagagcgtcaca |
| | | aggcatctctagttatctcgcgtggtaccaacagaagccaggtaaagcacctaaactgttgatttacacggcatcaacatt |
| | | gcagtcaggtgtcccctcccgatttagtggcagtggtagcggtacagattttactcttaccatttcatctcttcagccagaa |
| | | gattttgctacgtactactgtcaacaagaatgggctaaaccacgaacctttggacagggtacgaaggtcgaaataaaa |
| SEQ ID NO: 176 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWAKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 200 | DNA Light Chain | gatatacagatgacgcaaagtccctctagtctttctgcaagtgtcggggacagagttaccattacctgcagagcgtcaca |
| | | aggcatctctagttatctcgcgtggtaccaacagaagccaggtaaagcacctaaactgttgatttacacggcatcaacatt |
| | | gcagtcaggtgtcccctcccgatttagtggcagtggtagcggtacagattttactcttaccatttcatctcttcagccagaa |
| | | gattttgctacgtactactgtcaacaagaatgggctaaaccacgaacctttggacagggtacgaaggtcgaaataaaac |
| | | ggaccgttgccgccccctccgtcttcatcttccccccgtctgacgagcagctcaaatccggcacagcttctgtagtctgct |
| | | tgctgaataacttctacccaagagaagccaaagttcagtggaaggtcgataatgcattgcaatctggtaatagtcaggaa |
| | | tctgtgactgagcaggatagcaaagactcaacttacagcctctcttcaaccttgacgttgtccaaagcggattatgagaaa |
| | | cacaaggtgtacgcttgcgaggtgacgcatcaagggcttagttccccggtaaccaaatctttcaaccgaggtgaatgc |
| XX18_DAPA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 122 | VH | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 207 | DNA VH | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctcc |
| SEQ ID NO: 208 | Heavy Chain | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPSVFLFPPKP |
| | | KDTLMISRTPEVTCVVVAVSHEDPEVKFNWYVDGVEVHNAKTKPREEQY |
| | | NSTYRVVSVLTVLHQDWLNGKEYKCKVSNKALAAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 209 | DNA Heavy Chain | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctccgcctcaaccaagggccc |
| | | gtcagtgttcccgctggctccatcgtcgaagtccacctccggaggaaccgcagcactcggttgcctggtcaaggactac |
| | | ttccctgagccagtgaccgtgtcgtggaacagcggagccctgacttccggcgtgcacacttttcccgcggtgctgcagt |
| | | cctccggtctgtactccctttcgtccgtggtcaccgtgccgtcgtctagcctgggcacccagacctacatctgcaacgtg |
| | | aaccacaagccgtccaacaccaaagtggataagcgggtggagccgaagtcctgcgataagacacacacgtgcccgc |
| | | catgtccagcgcctgaattgcttggcggaccttccgtgttcctgttcccgcctaagcccaaggacaccttgatgattagcc |
| | | ggactcccgaagtcacctgtgtggtggtggcagtgtcccacgaggaccccgaggtcaagtttaattggtacgtggacg |
| | | gcgtcgaagtgcacaacgccaagactaagccccgggaggaacagtacaacagcacctaccgggtcgtgtccgtgct |
| | | gaccgtgctgcaccaggactggctgaatgggaaagagtacaagtgcaaagtgtccaacaaggccttggccgctcctat |
| | | cgaaaaaactatcagcaaggctaagggacagccgagggaaccccaagtctacaccctgcccccttcacgcgaagag |
| | | atgaccaagaatcaagtgtcgctgacctgcctcgtcaagggattctacccctccgacattgcggtggagtgggagtcca |
| | | acggccagcccgagaacaactacaagactactccgcccgtgctggactccgacggcagcttcttcctgtattccaagct |
| | | gaccgtggacaagtcccggtggcagcaaggaaacgtgttctcctgctcggtcatgcacgaagccctgcacaaccacta |
| | | tacgcagaagtccctgtccttgagcccggggaaa |
| SEQ ID NO: 41 | LCDR1 (Combined) | RASQGISSYLA |
| SEQ ID NO: 42 | LCDR2 (Combined) | TASTLQS |
| SEQ ID NO: 172 | LCDR3 (Combined) | QQEWAKPRT |
| SEQ ID NO: 41 | LCDR1 (Kabat) | RASQGISSYLA |
| SEQ ID NO: 42 | LCDR2 (Kabat) | TASTLQS |
| SEQ ID NO: 172 | LCDR3 (Kabat) | QQEWAKPRT |
| SEQ ID NO: 44 | LCDR1 (Chothia) | SQGISSY |
| SEQ ID NO: 45 | LCDR2 (Chothia) | TAS |
| SEQ ID NO: 173 | LCDR3 (Chothia) | EWAKPR |
| SEQ ID NO: 47 | LCDR1 (IMGT) | QGISSY |
| SEQ ID NO: 45 | LCDR2 (IMGT) | TAS |
| SEQ ID NO: 172 | LCDR3 (IMGT) | QQEWAKPRT |
| SEQ ID NO: 174 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWAKPRTFGQGTKV |
| | | EIK |
| SEQ ID NO: 216 | DNA VL | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaagaatgggccaagcccaggacatttggccagggcactaaggtcga |
| | | gattaag |
| SEQ ID NO: 176 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWAKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 217 | DNA Light Chain | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaagaatgggccaagcccaggacatttggccagggcactaaggtcga |
| | | gattaagcgtacggtggccgctcccagcgtgttcatcttcccccccagcgacgagcagctgaagagcggcaccgcca |
| | | gcgtggtgtgcctgctgaacaacttctacccccgggaggccaaggtgcagtggaaggtggacaacgccctgcagag |
| | | cggcaacagccaggagagcgtcaccgagcaggacagcaaggactccacctacagcctgagcagcaccctgaccct |
| | | gagcaaggccgactacgagaagcataaggtgtacgcctgcgaggtgacccaccagggcctgtccagccccgtgacc |
| | | aagagcttcaacaggggcgagtgc |
| XX19_LALA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 201 | VH | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 202 | DNA VH | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagc |
| SEQ ID NO: 203 | Heavy Chain | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 204 | DNA Heavy Chain | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagcgcctcaacgaaaggaccgtccgtgttt |
| | | cctcttgctcctagctccaaatccacctcaggtggaacggccgccctggggtgcctggtaaaggactatttcccagagc |
| | | cagttactgtgtcttggaattctggtgcattgacaagtggcgtacacacttttcccgcggtcctccaatctagtggtctgtac |
| | | tcactgtcctccgttgtgactgtcccaagtagctcacttggcacacagacttacatctgtaatgttaatcataagccgtcaaa |
| | | cacgaaggtggataagagggtagaacctaagtcatgtgacaaaacgcatacttgccccccctgccctgcgccggaag |
| | | ccgctggcggaccctccgtattcttgttccctccaaagccaaaggacactctgatgattagccggacaccggaggtcac |
| | | ttgtgttgtagttgacgtcagccatgaggatcctgaggtgaaatttaattggtacgtggacggggttgaagtccacaatgc |
| | | taaaactaaacctagggaagagcaatataatagtacatacagggttgtcagtgtgctgaccgttctccatcaggactggc |
| | | tgaacggcaaggaatacaagtgcaaggtcagcaacaaggccttgccggcccccatcgagaagacgatctccaaagc |
| | | caaggggcaaccccgagaaccgcaggtatacacgctcccccctagtagagaagagatgacaaagaatcaagtttcctt |
| | | gacgtgccttgtgaaaggcttctaccctagtgacatcgcagtcgaatgggagagcaacgggcagccggagaataacta |
| | | taaaacaaccccccccgtgcttgactcagacgggtcattttttctgtatagcaaattgactgttgataaatcacggtggcaa |
| | | caaggaaacgtgtttagttgcagcgtaatgcacgaagctctccacaatcactatactcaaaagtcactgtcactctcccct |
| | | ggcaag |
| SEQ ID NO: 41 | LCDR1 (Combined) | RASQGISSYLA |
| SEQ ID NO: 42 | LCDR2 (Combined) | TASTLQS |
| SEQ ID NO: 178 | LCDR3 (Combined) | QQSWTRPRT |
| SEQ ID NO: 41 | LCDR1 (Kabat) | RASQGISSYLA |
| SEQ ID NO: 42 | LCDR2 (Kabat) | TASTLQS |
| SEQ ID NO: 178 | LCDR3 (Kabat) | QQSWTRPRT |
| SEQ ID NO: 44 | LCDR1 (Chothia) | SQGISSY |
| SEQ ID NO: 45 | LCDR2 (Chothia) | TAS |
| SEQ ID NO: 179 | LCDR3 (Chothia) | SWTRPR |
| SEQ ID NO: 47 | LCDR1 (IMGT) | QGISSY |
| SEQ ID NO: 45 | LCDR2 (IMGT) | TAS |
| SEQ ID NO: 178 | LCDR3 (IMGT) | QQSWTRPRT |
| SEQ ID NO: 180 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQSWTRPRTFGQGTKV |
| | | EIK |
| SEQ ID NO: 218 | DNA VL | gatattcagatgacgcaatctccgtcttccttgtcagctagtgtaggagaccgcgtcacaattacctgtagagccagcca |
| | | ggggatttcctcataccttgcatggtaccagcaaaagccaggcaaagcccccaaactgctgatctacaccgcgtctacc |
| | | ttgcaatctggtgtgccgtcacgcttttccggctctggctcaggtactgatttcacattgacgatctcaagtctccagccgg |
| | | aagacttcgcaacttactactgccaacaatcctggacgaggccgaggactttcgggcagggaacaaaggttgaaatta |
| | | aa |
| SEQ ID NO: 182 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQSWTRPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 219 | DNA Light Chain | gatattcagatgacgcaatctccgtcttccttgtcagctagtgtaggagaccgcgtcacaattacctgtagagccagcca |
| | | ggggatttcctcataccttgcatggtaccagcaaaagccaggcaaagcccccaaactgctgatctacaccgcgtctacc |
| | | ttgcaatctggtgtgccgtcacgcttttccggctctggctcaggtactgatttcacattgacgatctcaagtctccagccgg |
| | | aagacttcgcaacttactactgccaacaatcctggacgaggccgaggactttcgggcagggaacaaaggttgaaatta |
| | | aaagaacagtcgcagcaccaagtgtttttatttttccaccctcagacgagcagctcaagtctggcaccgcgagcgtagta |
| | | tgtttgttgaataatttttaccctagggaagctaaggtacagtggaaagtggataatgctctccaaagtggcaactcccag |
| | | gaatcagtgactgagcaagattcaaaggacagcacgtattctctttcttctacgcttactctctctaaggccgactacgaaa |
| | | aacacaaagtttacgcttgcgaggttacccaccaggggctgtcctcaccagtaacgaaaagttttaaccggggcgagtg |
| | | t |
| XX19_DAPA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 122 | VH | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 207 | DNA VH | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctcc |
| SEQ ID NO: 208 | Heavy Chain | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPSVFLFPPKP |
| | | KDTLMISRTPEVTCVVVAVSHEDPEVKFNWYVDGVEVHNAKTKPREEQY |
| | | NSTYRVVSVLTVLHQDWLNGKEYKCKVSNKALAAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 209 | DNA Heavy Chain | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctccgcctcaaccaagggccc |
| | | gtcagtgttcccgctggctccatcgtcgaagtccacctccggaggaaccgcagcactcggttgcctggtcaaggactac |
| | | ttccctgagccagtgaccgtgtcgtggaacagcggagccctgacttccggcgtgcacacttttcccgcggtgctgcagt |
| | | cctccggtctgtactccctttcgtccgtggtcaccgtgccgtcgtctagcctgggcacccagacctacatctgcaacgtg |
| | | aaccacaagccgtccaacaccaaagtggataagcgggtggagccgaagtcctgcgataagacacacacgtgcccgc |
| | | catgtccagcgcctgaattgcttggcggaccttccgtgttcctgttcccgcctaagcccaaggacaccttgatgattagcc |
| | | ggactcccgaagtcacctgtgtggtggtggcagtgtcccacgaggaccccgaggtcaagtttaattggtacgtggacg |
| | | gcgtcgaagtgcacaacgccaagactaagccccgggaggaacagtacaacagcacctaccgggtcgtgtccgtgct |
| | | gaccgtgctgcaccaggactggctgaatgggaaagagtacaagtgcaaagtgtccaacaaggccttggccgctcctat |
| | | cgaaaaaactatcagcaaggctaagggacagccgagggaaccccaagtctacaccctgcccccttcacgcgaagag |
| | | atgaccaagaatcaagtgtcgctgacctgcctcgtcaagggattctacccctccgacattgcggtggagtgggagtcca |
| | | acggccagcccgagaacaactacaagactactccgcccgtgctggactccgacggcagcttcttcctgtattccaagct |
| | | gaccgtggacaagtcccggtggcagcaaggaaacgtgttctcctgctcggtcatgcacgaagccctgcacaaccacta |
| | | tacgcagaagtccctgtccttgagcccggggaaa |
| SEQ ID NO: 41 | LCDR1 (Combined) | RASQGISSYLA |
| SEQ ID NO: 42 | LCDR2 (Combined) | TASTLQS |
| SEQ ID NO: 178 | LCDR3 (Combined) | QQSWTRPRT |
| SEQ ID NO: 41 | LCDR1 (Kabat) | RASQGISSYLA |
| SEQ ID NO: 42 | LCDR2 (Kabat) | TASTLQS |
| SEQ ID NO: 178 | LCDR3 (Kabat) | QQSWTRPRT |
| SEQ ID NO: 44 | LCDR1 (Chothia) | SQGISSY |
| SEQ ID NO: 45 | LCDR2 (Chothia) | TAS |
| SEQ ID NO: 179 | LCDR3 (Chothia) | SWTRPR |
| SEQ ID NO: 47 | LCDR1 (IMGT) | QGISSY |
| SEQ ID NO: 45 | LCDR2 (IMGT) | TAS |
| SEQ ID NO: 178 | LCDR3 (IMGT) | QQSWTRPRT |
| SEQ ID NO: 180 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQSWTRPRTFGQGTKV |
| | | EIK |
| SEQ ID NO: 220 | DNA VL | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaagctggaccaggcccaggacatttggccagggcactaaggtcga |
| | | gattaag |
| SEQ ID NO: 182 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQSWTRPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 221 | DNA Light Chain | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaagctggaccaggcccaggacatttggccagggcactaaggtcga |
| | | gattaagcgtacggtggccgctcccagcgtgttcatcttcccccccagcgacgagcagctgaagagcggcaccgcca |
| | | gcgtggtgtgcctgctgaacaacttctacccccgggaggccaaggtgcagtggaaggtggacaacgccctgcagag |
| | | cggcaacagccaggagagcgtcaccgagcaggacagcaaggactccacctacagcctgagcagcaccctgaccct |
| | | gagcaaggccgactacgagaagcataaggtgtacgcctgcgaggtgacccaccagggcctgtccagccccgtgacc |
| | | aagagcttcaacaggggcgagtgc |
| XX20_LALA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 201 | VH | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 202 | DNA VH | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagc |
| SEQ ID NO: 203 | Heavy Chain | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 204 | DNA Heavy Chain | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagcgcctcaacgaaaggaccgtccgtgttt |
| | | cctcttgctcctagctccaaatccacctcaggtggaacggccgccctggggtgcctggtaaaggactatttcccagagc |
| | | cagttactgtgtcttggaattctggtgcattgacaagtggcgtacacacttttcccgcggtcctccaatctagtggtctgtac |
| | | tcactgtcctccgttgtgactgtcccaagtagctcacttggcacacagacttacatctgtaatgttaatcataagccgtcaaa |
| | | cacgaaggtggataagagggtagaacctaagtcatgtgacaaaacgcatacttgccccccctgccctgcgccggaag |
| | | ccgctggcggaccctccgtattcttgttccctccaaagccaaaggacactctgatgattagccggacaccggaggtcac |
| | | ttgtgttgtagttgacgtcagccatgaggatcctgaggtgaaatttaattggtacgtggacggggttgaagtccacaatgc |
| | | taaaactaaacctagggaagagcaatataatagtacatacagggttgtcagtgtgctgaccgttctccatcaggactggc |
| | | tgaacggcaaggaatacaagtgcaaggtcagcaacaaggccttgccggcccccatcgagaagacgatctccaaagc |
| | | caaggggcaaccccgagaaccgcaggtatacacgctcccccctagtagagaagagatgacaaagaatcaagtttcctt |
| | | gacgtgccttgtgaaaggcttctaccctagtgacatcgcagtcgaatgggagagcaacgggcagccggagaataacta |
| | | taaaacaaccccccccgtgcttgactcagacgggtcattttttctgtatagcaaattgactgttgataaatcacggtggcaa |
| | | caaggaaacgtgtttagttgcagcgtaatgcacgaagctctccacaatcactatactcaaaagtcactgtcactctcccct |
| | | ggcaag |
| SEQ ID NO: 41 | LCDR1 (Combined) | RASQGISSYLA |
| SEQ ID NO: 42 | LCDR2 (Combined) | TASTLQS |
| SEQ ID NO: 184 | LCDR3 (Combined) | QQIWMAPRT |
| SEQ ID NO: 41 | LCDR1 (Kabat) | RASQGISSYLA |
| SEQ ID NO: 42 | LCDR2 (Kabat) | TASTLQS |
| SEQ ID NO: 184 | LCDR3 (Kabat) | QQIWMAPRT |
| SEQ ID NO: 44 | LCDR1 (Chothia) | SQGISSY |
| SEQ ID NO: 45 | LCDR2 (Chothia) | TAS |
| SEQ ID NO: 185 | LCDR3 (Chothia) | IWMAPR |
| SEQ ID NO: 47 | LCDR1 (IMGT) | QGISSY |
| SEQ ID NO: 45 | LCDR2 (IMGT) | TAS |
| SEQ ID NO: 184 | LCDR3 (IMGT) | QQIWMAPRT |
| SEQ ID NO: 186 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWMAPRTFGQGTKV |
| | | EIK |
| SEQ ID NO: 222 | DNA VL | gacattcaaatgactcagtctccctcatctttgtcagcatcagttggggacagggtgacaatcacatgccgagcctcaca |
| | | ggggatttctagctatcttgcatggtaccaacagaagcccggcaaagcccccaagcttttgatatatacggcatccactct |
| | | tcagagcggagtacccagtaggtttagtggctccgggagtggtacggactttactctgacgatttcctcccttcaacctga |
| | | agactttgcaacgtattactgtcagcaaatatggatggctcccagaacgtttggtcaaggtactaaagttgaaataaag |
| SEQ ID NO: 188 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWMAPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 223 | DNA Light Chain | gacattcaaatgactcagtctccctcatctttgtcagcatcagttggggacagggtgacaatcacatgccgagcctcaca |
| | | ggggatttctagctatcttgcatggtaccaacagaagcccggcaaagcccccaagcttttgatatatacggcatccactct |
| | | tcagagcggagtacccagtaggtttagtggctccgggagtggtacggactttactctgacgatttcctcccttcaacctga |
| | | agactttgcaacgtattactgtcagcaaatatggatggctcccagaacgtttggtcaaggtactaaagttgaaataaagcg |
| | | aactgtagcagcacctagtgtatttatcttccccccttctgatgaacagttgaagtccgggacggcttccgtcgtatgtctc |
| | | ctgaacaacttttacccaagggaggcaaaggtgcaatggaaggtggataatgcactccagagtggcaatagccaaga |
| | | atcagtaaccgaacaggattccaaggattctacctacagcctttcctctacgcttacattgagcaaggcggactatgaaaa |
| | | gcataaggtgtatgcgtgcgaagtaacacaccagggtctcagcagtccagttacgaagtctttcaatcggggagaatgt |
| XX20_DAPA | | |
| SEQ ID NO: 28 | HCDR1 (Combined) | GFTFSSYWMN |
| SEQ ID NO: 119 | HCDR2 (Combined) | VIESKGNYIFYADSVKG |
| SEQ ID NO: 30 | HCDR3 (Combined) | DRYSMIYSYGAGAFDY |
| SEQ ID NO: 31 | HCDR1 (Kabat) | SYWMN |
| SEQ ID NO: 119 | HCDR2 (Kabat) | VIESKGNYIFYADSVKG |
| SEQ ID NO: 30 | HCDR3 (Kabat) | DRYSMIYSYGAGAFDY |
| SEQ ID NO: 32 | HCDR1 (Chothia) | GFTFSSY |
| SEQ ID NO: 120 | HCDR2 (Chothia) | ESKGNY |
| SEQ ID NO: 30 | HCDR3 (Chothia) | DRYSMIYSYGAGAFDY |
| SEQ ID NO: 34 | HCDR1 (IMGT) | GFTFSSYW |
| SEQ ID NO: 121 | HCDR2 (IMGT) | IESKGNYI |
| SEQ ID NO: 36 | HCDR3 (IMGT) | ARDRYSMIYSYGAGAFDY |
| SEQ ID NO: 122 | VH | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 207 | DNA VH | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctcc |
| SEQ ID NO: 208 | Heavy Chain | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPSVFLFPPKP |
| | | KDTLMISRTPEVTCVVVAVSHEDPEVKFNWYVDGVEVHNAKTKPREEQY |
| | | NSTYRVVSVLTVLHQDWLNGKEYKCKVSNKALAAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 209 | DNA Heavy Chain | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctccgcctcaaccaagggccc |
| | | gtcagtgttcccgctggctccatcgtcgaagtccacctccggaggaaccgcagcactcggttgcctggtcaaggactac |
| | | ttccctgagccagtgaccgtgtcgtggaacagcggagccctgacttccggcgtgcacacttttcccgcggtgctgcagt |
| | | cctccggtctgtactccctttcgtccgtggtcaccgtgccgtcgtctagcctgggcacccagacctacatctgcaacgtg |
| | | aaccacaagccgtccaacaccaaagtggataagcgggtggagccgaagtcctgcgataagacacacacgtgcccgc |
| | | catgtccagcgcctgaattgcttggcggaccttccgtgttcctgttcccgcctaagcccaaggacaccttgatgattagcc |
| | | ggactcccgaagtcacctgtgtggtggtggcagtgtcccacgaggaccccgaggtcaagtttaattggtacgtggacg |
| | | gcgtcgaagtgcacaacgccaagactaagccccgggaggaacagtacaacagcacctaccgggtcgtgtccgtgct |
| | | gaccgtgctgcaccaggactggctgaatgggaaagagtacaagtgcaaagtgtccaacaaggccttggccgctcctat |
| | | cgaaaaaactatcagcaaggctaagggacagccgagggaaccccaagtctacaccctgcccccttcacgcgaagag |
| | | atgaccaagaatcaagtgtcgctgacctgcctcgtcaagggattctacccctccgacattgcggtggagtgggagtcca |
| | | acggccagcccgagaacaactacaagactactccgcccgtgctggactccgacggcagcttcttcctgtattccaagct |
| | | gaccgtggacaagtcccggtggcagcaaggaaacgtgttctcctgctcggtcatgcacgaagccctgcacaaccacta |
| | | tacgcagaagtccctgtccttgagcccggggaaa |
| SEQ ID NO: 41 | LCDR1 (Combined) | RASQGISSYLA |
| SEQ ID NO: 42 | LCDR2 (Combined) | TASTLQS |
| SEQ ID NO: 184 | LCDR3 (Combined) | QQIWMAPRT |
| SEQ ID NO: 41 | LCDR1 (Kabat) | RASQGISSYLA |
| SEQ ID NO: 42 | LCDR2 (Kabat) | TASTLQS |
| SEQ ID NO: 184 | LCDR3 (Kabat) | QQIWMAPRT |
| SEQ ID NO: 44 | LCDR1 (Chothia) | SQGISSY |
| SEQ ID NO: 45 | LCDR2 (Chothia) | TAS |
| SEQ ID NO: 185 | LCDR3 (Chothia) | IWMAPR |
| SEQ ID NO: 47 | LCDR1 (IMGT) | QGISSY |
| SEQ ID NO: 45 | LCDR2 (IMGT) | TAS |
| SEQ ID NO: 184 | LCDR3 (IMGT) | QQIWMAPRT |
| SEQ ID NO: 186 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWMAPRTFGQGTKV |
| | | EIK |
| SEQ ID NO: 224 | DNA VL | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaatctggatggcccccaggacatttggccagggcactaaggtcgag |
| | | attaag |
| SEQ ID NO: 188 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQIWMAPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 225 | DNA Light Chain | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaatctggatggcccccaggacatttggccagggcactaaggtcgag |
| | | attaagcgtacggtggccgctcccagcgtgttcatcttcccccccagcgacgagcagctgaagagcggcaccgccag |
| | | cgtggtgtgcctgctgaacaacttctacccccgggaggccaaggtgcagtggaaggtggacaacgccctgcagagc |
| | | ggcaacagccaggagagcgtcaccgagcaggacagcaaggactccacctacagcctgagcagcaccctgaccctg |
| | | agcaaggccgactacgagaagcataaggtgtacgcctgcgaggtgacccaccagggcctgtccagccccgtgacca |
| | | agagcttcaacaggggcgagtgc |
| YY01_LALA | | |
| SEQ ID NO: 226 | HCDR1 (Combined) | GFTFSSYWIS |
| SEQ ID NO: 227 | HCDR2 (Combined) | NIKQSGSETYYVESVKG |
| SEQ ID NO: 228 | HCDR3 (Combined) | SLRRRSTEHAGFDV |
| SEQ ID NO: 229 | HCDR1 (Kabat) | SYWIS |
| SEQ ID NO: 227 | HCDR2 (Kabat) | NIKQSGSETYYVESVKG |
| SEQ ID NO: 228 | HCDR3 (Kabat) | SLRRRSTEHAGFDV |
| SEQ ID NO: 32 | HCDR1 (Chothia) | GFTFSSY |
| SEQ ID NO: 230 | HCDR2 (Chothia) | KQSGSE |
| SEQ ID NO: 228 | HCDR3 (Chothia) | SLRRRSTEHAGFDV |
| SEQ ID NO: 34 | HCDR1 (IMGT) | GFTFSSYW |
| SEQ ID NO: 231 | HCDR2 (IMGT) | IKQSGSET |
| SEQ ID NO: 232 | HCDR3 (IMGT) | ARSLRRRSTEHAGFDV |
| SEQ ID NO: 233 | VH | EVQLVESGGGLVQPGGSLRLSCAASGFTFSSYWISWVRQAPGKGLEWVAN |
| | | IKQSGSETYYVESVKGRFTISRDNAKNSLYLQMNSLRAEDTAVYYCARSLR |
| | | RRSTEHAGFDVWGQGTLVTVSS |
| SEQ ID NO: 234 | DNA VH | gaagtgcagctggtggaaagcggcggtggcctggtgcagccaggtggtagcctgcgcctgagctgcgccgccagc |
| | | ggctttacctttagcagctattggattagctgggttcgccaggccccaggcaaaggcctggaatgggggcgaacatca |
| | | aacagagcggcagcgagacctactatgtggagagcgtgaaaggccgctttaccattagccgcgataacgccaaaaac |
| | | agcctgtatctgcaaatgaacagcctgcgggccgaagataccgccgtgtattattgcgcgcgtagcctgcgtcgtcgta |
| | | gcactgagcacgcaggattcgacgtttggggccagggcaccctggttactgtctcgagc |
| SEQ ID NO: 235 | Heavy Chain | EVQLVESGGGLVQPGGSLRLSCAASGFTFSSYWISWVRQAPGKGLEWVAN |
| | | IKQSGSETYYVESVKGRFTISRDNAKNSLYLQMNSLRAEDTAVYYCARSLR |
| | | RRSTEHAGFDVWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTY |
| | | ICNVNHKPSNTKVDKRVEPKSCDKTHTCPPCPAPEAAGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 236 | DNA Heavy Chain | gaagtgcagctggtggaaagcggcggtggcctggtgcagccaggtggtagcctgcgcctgagctgcgccgccagc |
| | | ggctttacctttagcagctattggattagctgggttcgccaggccccaggcaaaggcctggaatgggtggcgaacatca |
| | | aacagagcggcagcgagacctactatgtggagagcgtgaaaggccgctttaccattagccgcgataacgccaaaaac |
| | | agcctgtatctgcaaatgaacagcctgcgggccgaagataccgccgtgtattattgcgcgcgtagcctgcgtcgtcgta |
| | | gcactgagcacgcaggattcgacgtttggggccagggcaccctggttactgtctcgagcgcgtcgaccaaaggcccc |
| | | agcgtgttccctctggcccccagcagcaagagcacctctggcggaacagccgccctgggctgcctggtcaaggacta |
| | | cttccccgagcccgtgaccgtgtcctggaactctggcgccctgaccagcggcgtgcacacctttccagccgtgctcca |
| | | gagcagcggcctgtacagcctgagcagcgtcgtgaccgtgcccagcagcagcctgggcacccagacctacatctgc |
| | | aacgtgaaccacaagcccagcaacacaaaggtggacaagcgggtggaacccaagagctgcgacaagacccacac |
| | | ctgtcccccctgccctgcccctgaagcgggggaggcccctccgtgttcctgttccccccaaagcctaaggacaccct |
| | | gatgatcagccggacccccgaagtgacctgcgtggtggtggacgtgtcccacgaggaccctgaagtgaagtttaattg |
| | | gtacgtggacggcgtggaagtgcacaacgccaagaccaagcccagagaggaacagtacaacagcacctaccgggt |
| | | ggtgtccgtgctgaccgtgctgcaccaggactggctgaacggcaaagagtacaagtgcaaggtgtccaacaaggccc |
| | | tgcctgcccccatcgagaaaaccatcagcaaggccaaaggccagccccgcgagccccaggtgtacacactgccccc |
| | | tagccgggaagagatgaccaagaaccaggtgtccctgacctgcctcgtgaagggcttctaccccagcgacattgccgt |
| | | ggaatgggagagcaacggccagcccgagaacaactacaagaccaccccccctgtgctggacagcgacggctcattc |
| | | ttcctgtacagcaagctgaccgtggacaagagccggtggcagcagggcaacgtgttcagctgctccgtgatgcacga |
| | | ggccctgcacaaccactacacccagaagtccctgagcctgagccccggcaag |
| SEQ ID NO: 237 | LCDR1 (Combined) | RASQGISNYLA |
| SEQ ID NO: 238 | LCDR2 (Combined) | AASTLQS |
| SEQ ID NO: 239 | LCDR3 (Combined) | QQADKFPYT |
| SEQ ID NO: 237 | LCDR1 (Kabat) | RASQGISNYLA |
| SEQ ID NO: 238 | LCDR2 (Kabat) | AASTLQS |
| SEQ ID NO: 239 | LCDR3 (Kabat) | QQADKFPYT |
| SEQ ID NO: 240 | LCDR1 (Chothia) | SQGISNY |
| SEQ ID NO: 241 | LCDR2 (Chothia) | AAS |
| SEQ ID NO: 242 | LCDR3 (Chothia) | ADKFPY |
| SEQ ID NO: 243 | LCDR1 (IMGT) | QGISNY |
| SEQ ID NO: 241 | LCDR2 (IMGT) | AAS |
| SEQ ID NO: 239 | LCDR3 (IMGT) | QQADKFPYT |
| SEQ ID NO: 244 | VL | DIQMTQSPSSLSASVGDRVTITCRASQGISNYLAWYQQKPGKVPKLLIYAAS |
| | | TLQSGVPSRFSGSGSGTDFTLTISSLQPEDVATYYCQQADKFPYTFGQGTKV |
| | | EIK |
| SEQ ID NO: 245 | DNA VL | gatattcagatgacccagagcccgagcagcctgagcgcaagcgtgggcgatcgcgtgaccattacctgccgcgcca |
| | | gccagggcattagcaactatctggcctggtatcagcagaaaccgggcaaagtgccgaaactgctgatctatgccgcca |
| | | gcaccctgcaaagcggcgtgccaagtcgctttagcggcagcggtagcggcaccgatttcaccctgaccattagcagc |
| | | ctgcaaccggaagacgtggcgacctattattgccagcaggctgacaaattcccgtacaccttcggccagggtaccaaa |
| | | gtggaaatcaag |
| SEQ ID NO: 246 | Light Chain | DIQMTQSPSSLSASVGDRVTITCRASQGISNYLAWYQQKPGKVPKLLIYAAS |
| | | TLQSGVPSRFSGSGSGTDFTLTISSLQPEDVATYYCQQADKFPYTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 247 | DNA Light Chain | gatattcagatgacccagagcccgagcagcctgagcgcaagcgtgggcgatcgcgtgaccattacctgccgcgcca |
| | | gccagggcattagcaactatctggcctggtatcagcagaaaccgggcaaagtgccgaaactgctgatctatgccgcca |
| | | gcaccctgcaaagcggcgtgccaagtcgctttagcggcagcggtagcggcaccgatttcaccctgaccattagcagc |
| | | ctgcaaccggaagacgtggcgacctattattgccagcaggctgacaaattcccgtacaccttcggccagggtaccaaa |
| | | gtggaaatcaagcggaccgtggccgctccctccgtgttcatcttcccacccagcgacgagcagctgaagtccggcaca |
| | | gccagcgtcgtgtgcctgctgaacaacttctacccccgcgaggccaaagtgcagtggaaggtggacaacgccctcca |
| | | gagcggcaacagccaggaaagcgtcaccgagcaggacagcaaggactccacctacagcctgagcagcaccctgac |
| | | cctgagcaaggccgactacgagaagcacaaggtgtacgcctgcgaagtgacccaccagggcctgtccagccccgtg |
| | | accaagagcttcaaccggggcgagtgt |
| YY02_LALA | | |
| SEQ ID NO: 248 | HCDR1 (Combined) | GFTFSSYSMN |
| SEQ ID NO: 249 | HCDR2 (Combined) | SISSSSSYIYYADSVKG |
| SEQ ID NO: 250 | HCDR3 (Combined) | SGYRGVYGFDY |
| SEQ ID NO: 251 | HCDR1 (Kabat) | SYSMN |
| SEQ ID NO: 249 | HCDR2 (Kabat) | SISSSSSYIYYADSVKG |
| SEQ ID NO: 250 | HCDR3 (Kabat) | SGYRGVYGFDY |
| SEQ ID NO: 32 | HCDR1 (Chothia) | GFTFSSY |
| SEQ ID NO: 252 | HCDR2 (Chothia) | SSSSSY |
| SEQ ID NO: 250 | HCDR3 (Chothia) | SGYRGVYGFDY |
| SEQ ID NO: 253 | HCDR1 (IMGT) | GFTFSSYS |
| SEQ ID NO: 254 | HCDR2 (IMGT) | ISSSSSYI |
| SEQ ID NO: 255 | HCDR3 (IMGT) | ARSGYRGVYGFDY |
| SEQ ID NO: 256 | VH | EVQLVESGGGLVKPGGSLRLSCAASGFTFSSYSMNWVRQAPGKGLEWVSS |
| | | ISSSSSYIYYADSVKGRFTISRDNAKNSLYLQMNSLRAEDTAVYYCARSGY |
| | | RGVYGFDYWGQGTLVTVSS |
| SEQ ID NO: 257 | DNA VH | gaagtgcagctggtggaaagcggcggtggcctggtgaaaccaggcggtagcctgcgcctgagctgcgccgccagc |
| | | ggctttacctttagcagctatagcatgaactgggttcgccaggccccaggcaaaggcctggaatgggttagcagcatca |
| | | gcagcagtagcagctatatctattacgccgatagcgtgaaaggccgctttaccattagccgcgataacgccaaaaacag |
| | | cctgtatctgcaaatgaacagcctgcgggccgaagataccgccgtgtattattgcgcgcgaagcggatatcgtggagtt |
| | | tacggatttgattattggggccagggcaccctggttactgtctcgagc |
| SEQ ID NO: 258 | Heavy Chain | EVQLVESGGGLVKPGGSLRLSCAASGFTFSSYSMNWVRQAPGKGLEWVSS |
| | | ISSSSSYIYYADSVKGRFTISRDNAKNSLYLQMNSLRAEDTAVYYCARSGY |
| | | RGVYGFDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQTYIC |
| | | NVNHKPSNTKVDKRVEPKSCDKTHTCPPCPAPEAAGGPSVFLFPPKPKDTL |
| | | MISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTLP |
| | | PSREEMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| SEQ ID NO: 259 | DNA Heavy Chain | gaagtgcagctggtggaaagcggcggtggcctggtgaaaccaggcggtagcctgcgcctgagctgcgccgccagc |
| | | ggctttacctttagcagctatagcatgaactgggttcgccaggccccaggcaaaggcctggaatgggttagcagcatca |
| | | gcagcagtagcagctatatctattacgccgatagcgtgaaaggccgctttaccattagccgcgataacgccaaaaacag |
| | | cctgtatctgcaaatgaacagcctgcgggccgaagataccgccgtgtattattgcgcgcgaagcggatatcgtggagtt |
| | | tacggatttgattattggggccagggcaccctggttactgtctcgagcgcgaccaaaggccccagcgtgttccctct |
| | | ggcccccagcagcaagagcacctctggcggaacagccgccctgggctgcctggtcaaggactacttccccgagccc |
| | | gtgaccgtgtcctggaactctggcgccctgaccagcggcgtgcacacctttccagccgtgctccagagcagcggcctg |
| | | tacagcctgagcagcgtcgtgaccgtgcccagcagcagcctgggcacccagacctacatctgcaacgtgaaccacaa |
| | | gcccagcaacacaaaggtggacaagcgggtggaacccaagagctgcgacaagacccacacctgtcccccctgccct |
| | | gcccctgaagcgggggaggcccctccgtgttcctgttccccccaaagcctaaggacaccctgatgatcagccggac |
| | | ccccgaagtgacctgcgtggtggtggacgtgtcccacgaggaccctgaagtgaagtttaattggtacgtggacggcgt |
| | | ggaagtgcacaacgccaagaccaagcccagagaggaacagtacaacagcacctaccgggtggtgtccgtgctgacc |
| | | gtgctgcaccaggactggctgaacggcaaagagtacaagtgcaaggtgtccaacaaggccctgcctgcccccatcga |
| | | gaaaaccatcagcaaggccaaaggccagccccgcgagccccaggtgtacacactgccccctagccgggaagagat |
| | | gaccaagaaccaggtgtccctgacctgcctcgtgaagggcttctaccccagcgacattgccgtggaatgggagagca |
| | | acggccagcccgagaacaactacaagaccaccccccctgtgctggacagcgacggctcattcttcctgtacagcaag |
| | | ctgaccgtggacaagagccggtggcagcagggcaacgtgttcagctgctccgtgatgcacgaggccctgcacaacc |
| | | actacacccagaagtccctgagcctgagccccggcaag |
| SEQ ID NO: 260 | LCDR1 (Combined) | RASQGISSWLA |
| SEQ ID NO: 261 | LCDR2 (Combined) | AASSLQS |
| SEQ ID NO: 262 | LCDR3 (Combined) | QQYYHSPLT |
| SEQ ID NO: 260 | LCDR1 (Kabat) | RASQGISSWLA |
| SEQ ID NO: 261 | LCDR2 (Kabat) | AASSLQS |
| SEQ ID NO: 262 | LCDR3 (Kabat) | QQYYHSPLT |
| SEQ ID NO: 263 | LCDR1 (Chothia) | SQGISSW |
| SEQ ID NO: 241 | LCDR2 (Chothia) | AAS |
| SEQ ID NO: 264 | LCDR3 (Chothia) | YYHSPL |
| SEQ ID NO: 265 | LCDR1 (IMGT) | QGISSW |
| SEQ ID NO: 241 | LCDR2 (IMGT) | AAS |
| SEQ ID NO: 262 | LCDR3 (IMGT) | QQYYHSPLT |
| SEQ ID NO: 266 | VL | DIQMTQSPSSVSASVGDRVTITCRASQGISSWLAWYQQKPGKAPKLLIYAA |
| | | SSLQSGVPSRFSGSGSGTDFTLTISSLQPEDFATYYCQQYYHSPLTFGQGTK |
| | | VEIK |
| SEQ ID NO: 267 | DNA VL | gatattcagatgacccagagcccgagcagcgttagcgccagcgtgggcgatcgcgtgaccattacctgccgcgccag |
| | | tcagggcattagcagctggctggcctggtatcagcagaaaccgggcaaagccccgaaactgctgatctatgccgcca |
| | | gcagcctgcaaagcggcgtgccaagtcgctttagcggcagcggtagcggcaccgatttcaccctgaccattagcagtc |
| | | tgcaaccggaagactttgccacctattattgccagcagtactaccattctccgctgaccttcggccagggtaccaaagtg |
| | | gaaatcaag |
| SEQ ID NO: 268 | Light Chain | DIQMTQSPSSVSASVGDRVTITCRASQGISSWLAWYQQKPGKAPKLLIYAA |
| | | SSLQSGVPSRFSGSGSGTDFTLTISSLQPEDFATYYCQQYYHSPLTFGQGTK |
| | | VEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| SEQ ID NO: 269 | DNA Light Chain | gatattcagatgacccagagcccgagcagcgttagcgccagcgtgggcgatcgcgtgaccattacctgccgcgccag |
| | | tcagggcattagcagctggctggcctggtatcagcagaaaccgggcaaagccccgaaactgctgatctatgccgcca |
| | | gcagcctgcaaagcggcgtgccaagtcgctttagcggcagcggtagcggcaccgatttcaccctgaccattagcagtc |
| | | tgcaaccggaagactttgccacctattattgccagcagtactaccattctccgctgaccttcggccagggtaccaaagtg |
| | | gaaatcaagcggaccgtggccgctccctccgtgttcatcttcccacccagcgacgagcagctgaagtccggcacagc |
| | | cagcgtcgtgtgcctgctgaacaacttctacccccgcgaggccaaagtgcagtggaaggtggacaacgccctccaga |
| | | gcggcaacagccaggaaagcgtcaccgagcaggacagcaaggactccacctacagcctgagcagcaccctgaccc |
| | | tgagcaaggccgactacgagaagcacaaggtgtacgcctgcgaagtgacccaccagggcctgtccagccccgtgac |
| | | caagagcttcaaccggggcgagtgt |
| YY03_LALA | | |
| SEQ ID NO: 270 | HCDR1 (Combined) | GFTFSSYAIS |
| SEQ ID NO: 271 | HCDR2 (Combined) | AISGSGGSTYYAESVKG |
| SEQ ID NO: 272 | HCDR3 (Combined) | ESGYVYYLKFDY |
| SEQ ID NO: 273 | HCDR1 (Kabat) | SYAIS |
| SEQ ID NO: 271 | HCDR2 (Kabat) | AISGSGGSTYYAESVKG |
| SEQ ID NO: 272 | HCDR3 (Kabat) | ESGYVYYLKFDY |
| SEQ ID NO: 32 | HCDR1 (Chothia) | GFTFSSY |
| SEQ ID NO: 274 | HCDR2 (Chothia) | SGSGGS |
| SEQ ID NO: 272 | HCDR3 (Chothia) | ESGYVYYLKFDY |
| SEQ ID NO: 275 | HCDR1 (IMGT) | GFTFSSYA |
| SEQ ID NO: 276 | HCDR2 (IMGT) | ISGSGGST |
| SEQ ID NO: 277 | HCDR3 (IMGT) | ARESGYVYYLKFDY |
| SEQ ID NO: 278 | VH | EVQLLESGGGLVQPGGSLRLSCAASGFTFSSYAISWVRQAPGKGLEWVSAI |
| | | SGSGGSTYYAESVKGRFTISRDNSKNTLYLQMNSLRAEDTAVYYCARESG |
| | | YVYYLKFDYWGQGTLVTVSS |
| SEQ ID NO: 279 | DNA VH | gaagtgcagctgctggaaagcggtggcggtctggtgcagccaggtggtagcctgcgcctgagctgtgccgcaagcg |
| | | gctttacctttagcagctatgccattagctgggtgcgccaagcaccaggcaaaggcctggaatgggtgagcgccattag |
| | | cggcagcggtggcagcacctattatgccgagagcgtgaaaggtcgctttaccattagtcgcgataacagcaaaaacac |
| | | cctgtatctgcaaatgaacagcctgcgggcagaagataccgcagtttattattgcgcgcgtgagagcggatacgtttact |
| | | atctgaaattcgattattggggccagggcaccctggttactgtctcgagc |
| SEQ ID NO: 280 | Heavy Chain | EVQLLESGGGLVQPGGSLRLSCAASGFTFSSYAISWVRQAPGKGLEWVSAI |
| | | SGSGGSTYYAESVKGRFTISRDNSKNTLYLQMNSLRAEDTAVYYCARESG |
| | | YVYYLKFDYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKRVEPKSCDKTHTCPPCPAPEAAGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 281 | DNA Heavy Chain | gaagtgcagctgctggaaagcggtggcggtctggtgcagccaggtggtagcctgcgcctgagctgtgccgcaagcg |
| | | gctttacctttagcagctatgccattagctgggtgcgccaagcaccaggcaaaggcctggaatgggtgagcgccattag |
| | | cggcagcggtggcagcacctattatgccgagagcgtgaaaggtcgctttaccattagtcgcgataacagcaaaaacac |
| | | cctgtatctgcaaatgaacagcctgcgggcagaagataccgcagtttattattgcgcgcgtgagagcggatacgtttact |
| | | atctgaaattcgattattggggccagggcaccctggttactgtctcgagcgcgtcgaccaaaggccccagcgtgttccct |
| | | ctggcccccagcagcaagagcacctctggcggaacagccgccctgggctgcctggtcaaggactacttccccgagc |
| | | ccgtgaccgtgtcctggaactctggcgccctgaccagcggcgtgcacacctttccagccgtgctccagagcagcggc |
| | | ctgtacagcctgagcagcgtcgtgaccgtgcccagcagcagcctgggcacccagacctacatctgcaacgtgaacca |
| | | caagcccagcaacacaaaggtggacaagcgggtggaacccaagagctgcgacaagacccacacctgtcccccctg |
| | | ccctgcccctgaagcgggggaggcccctccgtgttcctgttccccccaaagcctaaggacaccctgatgatcagccg |
| | | gacccccgaagtgacctgcgtggtggtggacgtgtcccacgaggaccctgaagtgaagtttaattggtacgtggacgg |
| | | cgtggaagtgcacaacgccaagaccaagcccagagaggaacagtacaacagcacctaccgggtggtgtccgtgctg |
| | | accgtgctgcaccaggactggctgaacggcaaagagtacaagtgcaaggtgtccaacaaggccctgcctgcccccat |
| | | cgagaaaaccatcagcaaggccaaaggccagccccgcgagccccaggtgtacacactgccccctagccgggaaga |
| | | gatgaccaagaaccaggtgtccctgacctgcctcgtgaagggcttctaccccagcgacattgccgtggaatgggagag |
| | | caacggccagcccgagaacaactacaagaccaccccccctgtgctggacagcgacggctcattcttcctgtacagca |
| | | agctgaccgtggacaagagccggtggcagcagggcaacgtgttcagctgctccgtgatgcacgaggccctgcacaa |
| | | ccactacacccagaagtccctgagcctgagccccggcaag |
| SEQ ID NO: 282 | LCDR1 (Combined) | RASQSISSYLN |
| SEQ ID NO: 261 | LCDR2 (Combined) | AASSLQS |
| SEQ ID NO: 283 | LCDR3 (Combined) | QQHVRVPIT |
| SEQ ID NO: 282 | LCDR1 (Kabat) | RASQSISSYLN |
| SEQ ID NO: 261 | LCDR2 (Kabat) | AASSLQS |
| SEQ ID NO: 283 | LCDR3 (Kabat) | QQHVRVPIT |
| SEQ ID NO: 284 | LCDR1 (Chothia) | SQSISSY |
| SEQ ID NO: 241 | LCDR2 (Chothia) | AAS |
| SEQ ID NO: 285 | LCDR3 (Chothia) | HVRVPI |
| SEQ ID NO: 286 | LCDR1 (IMGT) | QSISSY |
| SEQ ID NO: 241 | LCDR2 (IMGT) | AAS |
| SEQ ID NO: 283 | LCDR3 (IMGT) | QQHVRVPIT |
| SEQ ID NO: 287 | VL | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQHVRVPITFGQGTKVE |
| | | IK |
| SEQ ID NO: 288 | DNA VL | gatattcagatgacccagagcccgagcagcctgagcgccagcgtgggtgatcgcgtgaccattacctgtcgcgcaag |
| | | ccagagcattagcagctatctgaactggtatcagcagaaaccaggcaaagccccaaaactgctgatttatgccgcaag |
| | | cagcctgcaaagcggtgtgccgagccgctttagcggcagcggtagcggcaccgattttaccctgaccattagtagcct |
| | | gcaaccggaagactttgccacctattattgccagcagcatgttcgtgttccgatcaccttcggccagggtaccaaagtgg |
| | | aaatcaag |
| SEQ ID NO: 289 | Light Chain | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQHVRVPITFGQGTKVE |
| | | IKRTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPVTK |
| | | SFNRGEC |
| SEQ ID NO: 290 | DNA Light Chain | gatattcagatgacccagagcccgagcagcctgagcgccagcgtgggtgatcgcgtgaccattacctgtcgcgcaag |
| | | ccagagcattagcagctatctgaactggtatcagcagaaaccaggcaaagccccaaaactgctgatttatgccgcaag |
| | | cagcctgcaaagcggtgtgccgagccgctttagcggcagcggtagcggcaccgattttaccctgaccattagtagcct |
| | | gcaaccggaagactttgccacctattattgccagcagcatgttcgtgttccgatcaccttcggccagggtaccaaagtgg |
| | | aaatcaagcggaccgtggccgctccctccgtgttcatcttcccacccagcgacgagcagctgaagtccggcacagcc |
| | | agcgtcgtgtgcctgctgaacaacttctacccccgcgaggccaaagtgcagtggaaggtggacaacgccctccagag |
| | | cggcaacagccaggaaagcgtcaccgagcaggacagcaaggactccacctacagcctgagcagcaccctgaccct |
| | | gagcaaggccgactacgagaagcacaaggtgtacgcctgcgaagtgacccaccagggcctgtccagccccgtgac |
| | | caagagcttcaaccggggcgagtgt |
| YY04_LALA | | |
| SEQ ID NO: 291 | HCDR1 (Combined) | GFTFSNYWIS |
| SEQ ID NO: 292 | HCDR2 (Combined) | RIKSKTYGGTTDYAEPVKG |
| SEQ ID NO: 293 | HCDR3 (Combined) | EKYSIRARGHGDYGFDV |
| SEQ ID NO: 294 | HCDR1 (Kabat) | NYWIS |
| SEQ ID NO: 292 | HCDR2 (Kabat) | RIKSKTYGGTTDYAEPVKG |
| SEQ ID NO: 293 | HCDR3 (Kabat) | EKYSIRARGHGDYGFDV |
| SEQ ID NO: 295 | HCDR1 (Chothia) | GFTFSNY |
| SEQ ID NO: 296 | HCDR2 (Chothia) | KSKTYGGT |
| SEQ ID NO: 293 | HCDR3 (Chothia) | EKYSIRARGHGDYGFDV |
| SEQ ID NO: 297 | HCDR1 (IMGT) | GFTFSNYW |
| SEQ ID NO: 298 | HCDR2 (IMGT) | IKSKTYGGTT |
| SEQ ID NO: 299 | HCDR3 (IMGT) | AREKYSIRARGHGDYGFDV |
| SEQ ID NO: 300 | VH | EVQLVESGGGLVKPGGSLRLSCAASGFTFSNYWISWVRQAPGKGLEWVGR |
| | | IKSKTYGGTTDYAEPVKGRFTISRDDSKNTLYLQMNSLKTEDTAVYYCARE |
| | | KYSIRARGHGDYGFDVWGQGTLVTVSS |
| SEQ ID NO: 301 | DNA VH | gaagtgcagctggtggaaagcggcggtggcctggtgaaaccaggcggtagcctgcgcctgagctgcgccgccagc |
| | | ggctttacctttagcaactattggattagctgggttcgccaggccccaggcaaaggcctggaatgggttggccgcatca |
| | | aaagcaaaacctatggcggcaccaccgattatgccgagccagtgaaaggccgctttaccattagccgcgacgatagc |
| | | aaaaacaccctgtacctgcaaatgaacagcctgaaaaccgaagataccgccgtgtattattgcgcgcgtgagaaatatt |
| | | ccatccgtgcacgtggtcacggagactacggatttgatgtgtggggccagggcaccctggttactgtctcgagc |
| SEQ ID NO: 302 | Heavy Chain | EVQLVESGGGLVKPGGSLRLSCAASGFTFSNYWISWVRQAPGKGLEWVGR |
| | | IKSKTYGGTTDYAEPVKGRFTISRDDSKNTLYLQMNSLKTEDTAVYYCARE |
| | | KYSIRARGHGDYGFDVWGQGTLVTVSSASTKGPSVFPLAPSSKSTSGGTAA |
| | | LGCLVKDYFPEPVTVSWNSGALTSGVHTFPAVLQSSGLYSLSSVVTVPSSSL |
| | | GTQTYICNVNHKPSNTKVDKRVEPKSCDKTHTCPPCPAPEAAGGPSVFLFP |
| | | PKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| SEQ ID NO: 303 | DNA Heavy Chain | gaagtgcagctggtggaaagcggcggtggcctggtgaaaccaggcggtagcctgcgcctgagctgcgccgccagc |
| | | ggctttacctttagcaactattggattagctgggttcgccaggccccaggcaaaggcctggaatgggttggccgcatca |
| | | aaagcaaaacctatggcggcaccaccgattatgccgagccagtgaaaggccgctttaccattagccgcgacgatagc |
| | | aaaaacaccctgtacctgcaaatgaacagcctgaaaaccgaagataccgccgtgtattattgcgcgcgtgagaaatatt |
| | | ccatccgtgcacgtggtcacggagactacggatttgatgtgtggggccagggcaccctggttactgtctcgagcgcgtc |
| | | gaccaaaggccccagcgtgttccctctggcccccagcagcaagagcacctctggcggaacagccgccctgggctgc |
| | | ctggtcaaggactacttccccgagcccgtgaccgtgtcctggaactctggcgccctgaccagcggcgtgcacacctttc |
| | | cagccgtgctccagagcagcggcctgtacagcctgagcagcgtcgtgaccgtgcccagcagcagcctgggcaccca |
| | | gacctacatctgcaacgtgaaccacaagcccagcaacacaaaggtggacaagcgggtggaacccaagagctgcga |
| | | caagacccacacctgtcccccctgccctgcccctgaagcgggggaggcccctccgtgttcctgttccccccaaagcc |
| | | taaggacaccctgatgatcagccggacccccgaagtgacctgcgtggtggtggacgtgtcccacgaggaccctgaag |
| | | tgaagtttaattggtacgtggacggcgtggaagtgcacaacgccaagaccaagcccagagaggaacagtacaacagc |
| | | acctaccgggtggtgtccgtgctgaccgtgctgcaccaggactggctgaacggcaaagagtacaagtgcaaggtgtc |
| | | caacaaggccctgcctgcccccatcgagaaaaccatcagcaaggccaaaggccagccccgcgagccccaggtgta |
| | | cacactgccccctagccgggaagagatgaccaagaaccaggtgtccctgacctgcctcgtgaagggcttctacccca |
| | | gcgacattgccgtggaatgggagagcaacggccagcccgagaacaactacaagaccaccccccctgtgctggacag |
| | | cgacggctcattcttcctgtacagcaagctgaccgtggacaagagccggtggcagcagggcaacgtgttcagctgctc |
| | | cgtgatgcacgaggccctgcacaaccactacacccagaagtccctgagcctgagccccggcaag |
| SEQ ID NO: 237 | LCDR1 (Combined) | RASQGISNYLA |
| SEQ ID NO: 238 | LCDR2 (Combined) | AASTLQS |
| SEQ ID NO: 304 | LCDR3 (Combined) | QQGYHAPFT |
| SEQ ID NO: 237 | LCDR1 (Kabat) | RASQGISNYLA |
| SEQ ID NO: 238 | LCDR2 (Kabat) | AASTLQS |
| SEQ ID NO: 304 | LCDR3 (Kabat) | QQGYHAPFT |
| SEQ ID NO: 240 | LCDR1 (Chothia) | SQGISNY |
| SEQ ID NO: 241 | LCDR2 (Chothia) | AAS |
| SEQ ID NO: 305 | LCDR3 (Chothia) | GYHAPF |
| SEQ ID NO: 243 | LCDR1 (IMGT) | QGISNY |
| SEQ ID NO: 241 | LCDR2 (IMGT) | AAS |
| SEQ ID NO: 304 | LCDR3 (IMGT) | QQGYHAPFT |
| SEQ ID NO: 306 | VL | DIQMTQSPSSLSASVGDRVTITCRASQGISNYLAWYQQKPGKVPKLLIYAAS |
| | | TLQSGVPSRFSGSGSGTDFTLTISSLQPEDVATYYCQQGYHAPFTFGQGTKV |
| | | EIK |
| SEQ ID NO: 307 | DNA VL | gatattcagatgacccagagcccgagcagcctgagcgcaagcgtgggcgatcgcgtgaccattacctgccgcgcca |
| | | gccagggcattagcaactatctggcctggtatcagcagaaaccgggcaaagtgccgaaactgctgatctatgccgcca |
| | | gcaccctgcaaagcggcgtgccaagtcgctttagcggcagcggtagcggcaccgatttcaccctgaccattagcagc |
| | | ctgcaaccggaagacgtggcgacctattattgccagcagggttaccatgctccgttcaccttcggccagggtaccaaag |
| | | tggaaatcaag |
| SEQ ID NO: 308 | Light Chain | DIQMTQSPSSLSASVGDRVTITCRASQGISNYLAWYQQKPGKVPKLLIYAAS |
| | | TLQSGVPSRFSGSGSGTDFTLTISSLQPEDVATYYCQQGYHAPFTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 309 | DNA Light Chain | gatattcagatgacccagagcccgagcagcctgagcgcaagcgtgggcgatcgcgtgaccattacctgccgcgcca |
| | | gccagggcattagcaactatctggcctggtatcagcagaaaccgggcaaagtgccgaaactgctgatctatgccgcca |
| | | gcaccctgcaaagcggcgtgccaagtcgctttagcggcagcggtagcggcaccgatttcaccctgaccattagcagc |
| | | ctgcaaccggaagacgtggcgacctattattgccagcagggttaccatgctccgttcaccttcggccagggtaccaaag |
| | | tggaaatcaagcggaccgtggccgctccctccgtgttcatcttcccacccagcgacgagcagctgaagtccggcacag |
| | | ccagcgtcgtgtgcctgctgaacaacttctacccccgcgaggccaaagtgcagtggaaggtggacaacgccctccag |
| | | agcggcaacagccaggaaagcgtcaccgagcaggacagcaaggactccacctacagcctgagcagcaccctgacc |
| | | ctgagcaaggccgactacgagaagcacaaggtgtacgcctgcgaagtgacccaccagggcctgtccagccccgtga |
| | | ccaagagcttcaaccggggcgagtgt |
| YY05_LALA | | |
| SEQ ID NO: 310 | HCDR1 (Combined) | GYSFTSYWIS |
| SEQ ID NO: 311 | HCDR2 (Combined) | IIYPGTSYTRYSPSFQG |
| SEQ ID NO: 312 | HCDR3 (Combined) | GAVAGQLGFDH |
| SEQ ID NO: 229 | HCDR1 (Kabat) | SYWIS |
| SEQ ID NO: 311 | HCDR2 (Kabat) | IIYPGTSYTRYSPSFQG |
| SEQ ID NO: 312 | HCDR3 (Kabat) | GAVAGQLGFDH |
| SEQ ID NO: 80 | HCDR1 (Chothia) | GYSFTSY |
| SEQ ID NO: 313 | HCDR2 (Chothia) | YPGTSY |
| SEQ ID NO: 312 | HCDR3 (Chothia) | GAVAGQLGFDH |
| SEQ ID NO: 82 | HCDR1 (IMGT) | GYSFTSYW |
| SEQ ID NO: 314 | HCDR2 (IMGT) | IYPGTSYT |
| SEQ ID NO: 315 | HCDR3 (IMGT) | ARGAVAGQLGFDH |
| SEQ ID NO: 316 | VH | EVQLVQSGAEVKKPGESLKISCKGSGYSFTSYWISWVRQMPGKGLEWMGII |
| | | YPGTSYTRYSPSFQGQVTISADKSISTAYLQWSSLKASDTAMYYCARGAVA |
| | | GQLGFDHWGQGTLVTVSS |
| SEQ ID NO: 317 | DNA VH | gaagtgcagctggtgcagagcggtgccgaagtgaaaaaaccgggcgaaagcctgaaaatcagctgcaaaggcagc |
| | | ggctatagctttaccagctattggattagctgggttcgccagatgccggcaaagctggaatggatgggcattatcta |
| | | tccgggcaccagctatacccgctatagcccgagctttcagggccaggttacaattagcgccgacaaaagcatcagcac |
| | | cgcctatctgcaatggagcagcctgaaagccagcgataccgccatgtattattgcgcgcgtggtgcagttgcaggaca |
| | | actgggatttgatcactggggccagggcaccctggttactgtctcgagc |
| SEQ ID NO: 318 | Heavy Chain | EVQLVQSGAEVKKPGESLKISCKGSGYSFTSYWISWVRQMPGKGLEWMGII |
| | | YPGTSYTRYSPSFQGQVTISADKSISTAYLQWSSLKASDTAMYYCARGAVA |
| | | GQLGFDHWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICNV |
| | | NHKPSNTKVDKRVEPKSCDKTHTCPPCPAPEAAGGPSVFLFPPKPKDTLMIS |
| | | RTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFF |
| | | LYSKLTVDKSRWQQGNVFSCSVMHEALHNHYTQKSLSLSPGK |
| SEQ ID NO: 319 | DNA Heavy Chain | gaagtgcagctggtgcagagcggtgccgaagtgaaaaaaccgggcgaaagcctgaaaatcagctgcaaaggcagc |
| | | ggctatagctttaccagctattggattagctgggttcgccagatgccgggcaaaggcctggaatggatgggcattatcta |
| | | tccgggcaccagctatacccgctatagcccgagctttcagggccaggttacaattagcgccgacaaaagcatcagcac |
| | | cgcctatctgcaatggagcagcctgaaagccagcgataccgccatgtattattgcgcgcgtggtgcagttgcaggaca |
| | | actgggatttgatcactggggccagggcaccctggttactgtctcgagcgcgtcgaccaaaggccccagcgtgttccct |
| | | ctggcccccagcagcaagagcacctctggcggaacagccgccctgggctgcctggtcaaggactacttccccgagc |
| | | ccgtgaccgtgtcctggaactctggcgccctgaccagcggcgtgcacacctttccagccgtgctccagagcagcggc |
| | | ctgtacagcctgagcagcgtcgtgaccgtgcccagcagcagcctgggcacccagacctacatctgcaacgtgaacca |
| | | caagcccagcaacacaaaggtggacaagcgggtggaacccaagagctgcgacaagacccacacctgtcccccctg |
| | | ccctgcccctgaagcgggggaggcccctccgtgttcctgttccccccaaagcctaaggacaccctgatgatcagccg |
| | | gacccccgaagtgacctgcgtggtggtggacgtgtcccacgaggaccctgaagtgaagtttaattggtacgtggacgg |
| | | cgtggaagtgcacaacgccaagaccaagcccagagaggaacagtacaacagcacctaccgggtggtgtccgtgctg |
| | | accgtgctgcaccaggactggctgaacggcaaagagtacaagtgcaaggtgtccaacaaggccctgcctgcccccat |
| | | cgagaaaaccatcagcaaggccaaaggccagccccgcgagccccaggtgtacacactgccccctagccgggaaga |
| | | gatgaccaagaaccaggtgtccctgacctgcctcgtgaagggcttctaccccagcgacattgccgtggaatgggagag |
| | | caacggccagcccgagaacaactacaagaccaccccccctgtgctggacagcgacggctcattcttcctgtacagca |
| | | agctgaccgtggacaagagccggtggcagcagggcaacgtgttcagctgctccgtgatgcacgaggccctgcacaa |
| | | ccactacacccagaagtccctgagcctgagccccggcaag |
| SEQ ID NO: 320 | LCDR1 (Combined) | TGSSSNIGAGYDVH |
| SEQ ID NO: 321 | LCDR2 (Combined) | GNSNRPS |
| SEQ ID NO: 322 | LCDR3 (Combined) | QSYYTSSHGPV |
| SEQ ID NO: 320 | LCDR1 (Kabat) | TGSSSNIGAGYDVH |
| SEQ ID NO: 321 | LCDR2 (Kabat) | GNSNRPS |
| SEQ ID NO: 322 | LCDR3 (Kabat) | QSYYTSSHGPV |
| SEQ ID NO: 323 | LCDR1 (Chothia) | SSSNIGAGYD |
| SEQ ID NO: 324 | LCDR2 (Chothia) | GNS |
| SEQ ID NO: 325 | LCDR3 (Chothia) | YYTSSHGP |
| SEQ ID NO: 326 | LCDR1 (IMGT) | SSNIGAGYD |
| SEQ ID NO: 324 | LCDR2 (IMGT) | GNS |
| SEQ ID NO: 322 | LCDR3 (IMGT) | QSYYTSSHGPV |
| SEQ ID NO: 327 | VL | QSVLTQPPSVSGAPGQRVTISCTGSSSNIGAGYDVHWYQQLPGTAPKLLIYG |
| | | NSNRPSGVPDRFSGSKSGTSASLAITGLQAEDEADYYCQSYYTSSHGPVFG |
| | | GGTKLTVL |
| SEQ ID NO: 328 | DNA VL | cagagcgtgctgacccagccaccaagcgtgagcggtgcaccaggtcagcgcgtgaccattagctgcaccggcagca |
| | | gcagcaacattggcgcaggctatgatgtgcattggtatcagcagctgccaggcaccgcaccgaaactgctgatttatgg |
| | | caacagcaatcgcccaagcggtgtgccggatcgctttagcggcagcaaaagcggcaccagcgccagcctggcgatt |
| | | accggtctgcaagccgaagacgaagccgattattactgccagtcttactacacttcttctcatggtccggtgtttggcggc |
| | | ggtaccaagctgaccgtgctg |
| SEQ ID NO: 329 | Light Chain | QSVLTQPPSVSGAPGQRVTISCTGSSSNIGAGYDVHWYQQLPGTAPKLLIYG |
| | | NSNRPSGVPDRFSGSKSGTSASLAITGLQAEDEADYYCQSYYTSSHGPVFG |
| | | GGTKLTVLGQPKAAPSVTLFPPSSEELQANKATLVCLISDFYPGAVTVAWK |
| | | ADSSPVKAGVETTTPSKQSNNKYAASSYLSLTPEQWKSHRSYSCQVTHEGS |
| | | TVEKTVAPTECS |
| SEQ ID NO: 330 | DNA Light Chain | cagagcgtgctgacccagccaccaagcgtgagcggtgcaccaggtcagcgcgtgaccattagctgcaccggcagca |
| | | gcagcaacattggcgcaggctatgatgtgcattggtatcagcagctgccaggcaccgcaccgaaactgctgatttatgg |
| | | caacagcaatcgcccaagcggtgtgccggatcgctttagcggcagcaaaagcggcaccagcgccagcctggcgatt |
| | | accggtctgcaagccgaagacgaagccgattattactgccagtcttactacacttcttctcatggtccggtgtttggcggc |
| | | ggtaccaagctgaccgtgctgggccagcccaaagccgcccctagcgtgaccctgttccccccaagcagcgaggaac |
| | | tccaggccaacaaggccaccctcgtgtgcctgatcagcgacttctaccctggcgccgtgaccgtggcctggaaggcc |
| | | gatagcagccctgtgaaggccggcgtggaaaccaccacccccagcaagcagagcaacaacaaatacgccgccagc |
| | | agctacctgagcctgacccccgagcagtggaagtcccacagatcctacagctgccaggtcacacacgagggcagca |
| | | ccgtggaaaagaccgtggcccccaccgagtgcagc |
| YY06_LALA | | |
| SEQ ID NO: 270 | HCDR1 (Combined) | GFTFSSYAIS |
| SEQ ID NO: 271 | HCDR2 (Combined) | AISGSGGSTYYAESVKG |
| SEQ ID NO: 331 | HCDR3 (Combined) | PYLGDRRSYGFDH |
| SEQ ID NO: 273 | HCDR1 (Kabat) | SYAIS |
| SEQ ID NO: 271 | HCDR2 (Kabat) | AISGSGGSTYYAESVKG |
| SEQ ID NO: 331 | HCDR3 (Kabat) | PYLGDRRSYGFDH |
| SEQ ID NO: 32 | HCDR1 (Chothia) | GFTFSSY |
| SEQ ID NO: 274 | HCDR2 (Chothia) | SGSGGS |
| SEQ ID NO: 331 | HCDR3 (Chothia) | PYLGDRRSYGFDH |
| SEQ ID NO: 275 | HCDR1 (IMGT) | GFTFSSYA |
| SEQ ID NO: 276 | HCDR2 (IMGT) | ISGSGGST |
| SEQ ID NO: 332 | HCDR3 (IMGT) | ARPYLGDRRSYGFDH |
| SEQ ID NO: 333 | VH | EVQLLESGGGLVQPGGSLRLSCAASGFTFSSYAISWVRQAPGKGLEWVSAI |
| | | SGSGGSTYYAESVKGRFTISRDNSKNTLYLQMNSLRAEDTAVYYCARPYL |
| | | GDRRSYGFDHWGQGTLVTVSS |
| SEQ ID NO: 334 | DNA VH | gaagtgcagctgctggaaagcggtggcggtctggtgcagccaggtggtagcctgcgcctgagctgtgccgcaagcg |
| | | gctttacctttagcagctatgccattagctgggtgcgccaagcaccaggcaaaggcctggaatgggtgagcgccattag |
| | | cggcagcggtggcagcacctattatgccgagagcgtgaaaggtcgctttaccattagtcgcgataacagcaaaaacac |
| | | cctgtatctgcaaatgaacagcctgcgggcagaagataccgcagtttattattgcgcgcgaccttatctgggtgaccgtc |
| | | gtagctatggtttcgaccactggggccagggcaccctggttactgtctcgagc |
| SEQ ID NO: 335 | Heavy Chain | EVQLLESGGGLVQPGGSLRLSCAASGFTFSSYAISWVRQAPGKGLEWVSAI |
| | | SGSGGSTYYAESVKGRFTISRDNSKNTLYLQMNSLRAEDTAVYYCARPYL |
| | | GDRRSYGFDHWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKRVEPKSCDKTHTCPPCPAPEAAGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 336 | DNA Heavy Chain | gaagtgcagctgctggaaagcggtggcggtctggtgcagccaggtggtagcctgcgcctgagctgtgccgcaagcg |
| | | gctttacctttagcagctatgccattagctgggtgcgccaagcaccaggcaaaggcctggaatgggtgagcgccattag |
| | | cggcagcggtggcagcacctattatgccgagagcgtgaaaggtcgctttaccattagtcgcgataacagcaaaaacac |
| | | cctgtatctgcaaatgaacagcctgcgggcagaagataccgcagtttattattgcgcgcgaccttatctgggtgaccgtc |
| | | gtagctatggtttcgaccactggggccagggcaccctggttactgtctcgagcgcgtcgaccaaaggccccagcgtgtt |
| | | ccctctggcccccagcagcaagagcacctctggcggaacagccgccctgggctgcctggtcaaggactacttccccg |
| | | agcccgtgaccgtgtcctggaactctggcgccctgaccagcggcgtgcacacctttccagccgtgctccagagcagc |
| | | ggcctgtacagcctgagcagcgtcgtgaccgtgcccagcagcagcctgggcacccagacctacatctgcaacgtgaa |
| | | ccacaagcccagcaacacaaaggggacaagcgggtggaacccaagagctgcgacaagacccacacctgtccccc |
| | | ctgccctgcccctgaagcggcgggaggcccctccgtgttcctgttccccccaaagcctaaggacaccctgatgatcag |
| | | ccggacccccgaagtgacctgcgtggtggtggacgtgtcccacgaggaccctgaagtgaagtttaattggtacgtgga |
| | | cggcgtggaagtgcacaacgccaagaccaagcccagagaggaacagtacaacagcacctaccgggtggtgtccgt |
| | | gctgaccgtgctgcaccaggactggctgaacggcaaagagtacaagtgcaaggtgtccaacaaggccctgcctgccc |
| | | ccatcgagaaaaccatcagcaaggccaaaggccagccccgcgagccccaggtgtacacactgccccctagccggg |
| | | aagagatgaccaagaaccaggtgtccctgacctgcctcgtgaagggcttctaccccagcgacattgccgtggaatggg |
| | | agagcaacggccagcccgagaacaactacaagaccaccccccctgtgctggacagcgacggctcattcttcctgtac |
| | | agcaagctgaccgtggacaagagccggtggcagcagggcaacgtgttcagctgctccgtgatgcacgaggccctgc |
| | | acaaccactacacccagaagtccctgagcctgagccccggcaag |
| SEQ ID NO: 337 | LCDR1 (Combined) | TGTSSDVGSYNLVS |
| SEQ ID NO: 338 | LCDR2 (Combined) | EGSKRPS |
| SEQ ID NO: 339 | LCDR3 (Combined) | SSYGFHIVVVV |
| SEQ ID NO: 337 | LCDR1 (Kabat) | TGTSSDVGSYNLVS |
| SEQ ID NO: 338 | LCDR2 (Kabat) | EGSKRPS |
| SEQ ID NO: 339 | LCDR3 (Kabat) | SSYGFHIVVVV |
| SEQ ID NO: 340 | LCDR1 (Chothia) | TSSDVGSYNL |
| SEQ ID NO: 341 | LCDR2 (Chothia) | EGS |
| SEQ ID NO: 342 | LCDR3 (Chothia) | YGFHIVVV |
| SEQ ID NO: 343 | LCDR1 (IMGT) | SSDVGSYNL |
| SEQ ID NO: 341 | LCDR2 (IMGT) | EGS |
| SEQ ID NO: 339 | LCDR3 (IMGT) | SSYGFHIVVVV |
| SEQ ID NO: 344 | VL | QSALTQPASVSGSPGQSITISCTGTSSDVGSYNLVSWYQQHPGKAPKLMIYE |
| | | GSKRPSGVSNRFSGSKSGNTASLTISGLQAEDEADYYCSSYGFHIVVVVFGG |
| | | GTKLTVL |
| SEQ ID NO: 345 | DNA VL | cagagcgccctgacccagccagccagcgttagcggtagcccaggccagagcattaccattagctgcaccggcacca |
| | | gcagcgacgtgggcagctataacctggttagctggtatcagcagcatccgggcaaagccccgaaactgatgatctatg |
| | | aaggcagcaaacgcccgagcggcgttagcaaccgctttagtggcagcaaaagcggcaacaccgccagcctgaccat |
| | | tagcggcctgcaagccgaagacgaagccgattattactgctcctcttacggtttccatatcgttgttgttgtgtttggcggc |
| | | ggtaccaagctgaccgtgctg |
| SEQ ID NO: 346 | Light Chain | QSALTQPASVSGSPGQSITISCTGTSSDVGSYNLVSWYQQHPGKAPKLMIYE |
| | | GSKRPSGVSNRFSGSKSGNTASLTISGLQAEDEADYYCSSYGFHIVVVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| SEQ ID NO: 347 | DNA Light Chain | cagagcgccctgacccagccagccagcgttagcggtagcccaggccagagcattaccattagctgcaccggcacca |
| | | gcagcgacgtgggcagctataacctggttagctggtatcagcagcatccgggcaaagccccgaaactgatgatctatg |
| | | aaggcagcaaacgcccgagcggcgttagcaaccgctttagtggcagcaaaagcggcaacaccgccagcctgaccat |
| | | tagcggcctgcaagccgaagacgaagccgattattactgctcctcttacggtttccatatcgttgttgttgtgtttggcggc |
| | | ggtaccaagctgaccgtgctgggccagcccaaagccgcccctagcgtgaccctgttccccccaagcagcgaggaac |
| | | tccaggccaacaaggccaccctcgtgtgcctgatcagcgacttctaccctggcgccgtgaccgtggcctggaaggcc |
| | | gatagcagccctgtgaaggccggcgtggaaaccaccacccccagcaagcagagcaacaacaaatacgccgccagc |
| | | agctacctgagcctgacccccgagcagtggaagtcccacagatcctacagctgccaggtcacacacgagggcagca |
| | | ccgtggaaaagaccgtggcccccaccgagtgcagc |
| YY07_LALA | | |
| SEQ ID NO: 310 | HCDR1 (Combined) | GYSFTSYWIS |
| SEQ ID NO: 311 | HCDR2 (Combined) | IIYPGTSYTRYSPSFQG |
| SEQ ID NO: 348 | HCDR3 (Combined) | GSLPGLLGFDH |
| SEQ ID NO: 229 | HCDR1 (Kabat) | SYWIS |
| SEQ ID NO: 311 | HCDR2 (Kabat) | IIYPGTSYTRYSPSFQG |
| SEQ ID NO: 348 | HCDR3 (Kabat) | GSLPGLLGFDH |
| SEQ ID NO: 80 | HCDR1 (Chothia) | GYSFTSY |
| SEQ ID NO: 313 | HCDR2 (Chothia) | YPGTSY |
| SEQ ID NO: 348 | HCDR3 (Chothia) | GSLPGLLGFDH |
| SEQ ID NO: 82 | HCDR1 (IMGT) | GYSFTSYW |
| SEQ ID NO: 314 | HCDR2 (IMGT) | IYPGTSYT |
| SEQ ID NO: 349 | HCDR3 (IMGT) | ARGSLPGLLGFDH |
| SEQ ID NO: 350 | VH | EVQLVQSGAEVKKPGESLKISCKGSGYSFTSYWISWVRQMPGKGLEWMGII |
| | | YPGTSYTRYSPSFQGQVTISADKSISTAYLQWSSLKASDTAMYYCARGSLP |
| | | GLLGFDHWGQGTLVTVSS |
| SEQ ID NO: 351 | DNA VH | gaagtgcagctggtgcagagcggtgccgaagtgaaaaaaccgggcgaaagcctgaaaatcagctgcaaaggcagc |
| | | ggctatagctttaccagctattggattagctgggttcgccagatgccgggcaaaggcctggaatggatgggcattatcta |
| | | tccgggcaccagctatacccgctatagcccgagctttcagggccaggttacaattagcgccgacaaaagcatcagcac |
| | | cgcctatctgcaatggagcagcctgaaagccagcgataccgccatgtattattgcgcgcgtggaagcctgcctggtctg |
| | | ctgggttttgatcactggggccagggcaccctggttactgtctcgagc |
| SEQ ID NO: 352 | Heavy Chain | EVQLVQSGAEVKKPGESLKISCKGSGYSFTSYWISWVRQMPGKGLEWMGII |
| | | YPGTSYTRYSPSFQGQVTISADKSISTAYLQWSSLKASDTAMYYCARGSLP |
| | | GLLGFDHWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICNV |
| | | NHKPSNTKVDKRVEPKSCDKTHTCPPCPAPEAAGGPSVFLFPPKPKDTLMIS |
| | | RTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFF |
| | | LYSKLTVDKSRWQQGNVFSCSVMHEALHNHYTQKSLSLSPGK |
| SEQ ID NO: 353 | DNA Heavy Chain | gaagtgcagctggtgcagagcggtgccgaagtgaaaaaaccgggcgaaagcctgaaaatcagctgcaaaggcagc |
| | | ggctatagctttaccagctattggattagctgggttcgccagatgccgggcaaaggcctggaatggatgggcattatcta |
| | | tccgggcaccagctatacccgctatagcccgagctttcagggccaggttacaattagcgccgacaaaagcatcagcac |
| | | cgcctatctgcaatggagcagcctgaaagccagcgataccgccatgtattattgcgcgcgtggaagcctgcctggtctg |
| | | ctgggttttgatcactggggccagggcaccctggttactgtctcgagcgcgtcgaccaaaggccccagcgtgttccctc |
| | | tggcccccagcagcaagagcacctctggcggaacagccgccctgggctgcctggtcaaggactacttccccgagcc |
| | | cgtgaccgtgtcctggaactctggcgccctgaccagcggcgtgcacacctttccagccgtgctccagagcagcggcct |
| | | gtacagcctgagcagcgtcgtgaccgtgcccagcagcagcctgggcacccagacctacatctgcaacgtgaaccaca |
| | | agcccagcaacacaaaggtggacaagcgggtggaacccaagagctgcgacaagacccacacctgtcccccctgcc |
| | | ctgcccctgaagcgggggaggcccctccgtgttcctgttccccccaaagcctaaggacaccctgatgatcagccgga |
| | | cccccgaagtgacctgcgtggtggtggacgtgtcccacgaggaccctgaagtgaagtttaattggtacgtggacggcg |
| | | tggaagtgcacaacgccaagaccaagcccagagaggaacagtacaacagcacctaccgggtggtgtccgtgctgac |
| | | cgtgctgcaccaggactggctgaacggcaaagagtacaagtgcaaggtgtccaacaaggccctgcctgcccccatcg |
| | | agaaaaccatcagcaaggccaaaggccagccccgcgagccccaggtgtacacactgccccctagccgggaagaga |
| | | tgaccaagaaccaggtgtccctgacctgcctcgtgaagggcttctaccccagcgacattgccgtggaatgggagagca |
| | | acggccagcccgagaacaactacaagaccaccccccctgtgctggacagcgacggctcattcttcctgtacagcaag |
| | | ctgaccgtggacaagagccggtggcagcagggcaacgtgttcagctgctccgtgatgcacgaggccctgcacaacc |
| | | actacacccagaagtccctgagcctgagccccggcaag |
| SEQ ID NO: 320 | LCDR1 (Combined) | TGSSSNIGAGYDVH |
| SEQ ID NO: 354 | LCDR2 (Combined) | GNSNRPN |
| SEQ ID NO: 355 | LCDR3 (Combined) | QSYDSPTSSSV |
| SEQ ID NO: 320 | LCDR1 (Kabat) | TGSSSNIGAGYDVH |
| SEQ ID NO: 354 | LCDR2 (Kabat) | GNSNRPN |
| SEQ ID NO: 355 | LCDR3 (Kabat) | QSYDSPTSSSV |
| SEQ ID NO: 323 | LCDR1 (Chothia) | SSSNIGAGYD |
| SEQ ID NO: 324 | LCDR2 (Chothia) | GNS |
| SEQ ID NO: 356 | LCDR3 (Chothia) | YDSPTSSS |
| SEQ ID NO: 326 | LCDR1 (IMGT) | SSNIGAGYD |
| SEQ ID NO: 324 | LCDR2 (IMGT) | GNS |
| SEQ ID NO: 355 | LCDR3 (IMGT) | QSYDSPTSSSV |
| SEQ ID NO: 357 | VL | QSVLTQPPSVSGAPGQRVTISCTGSSSNIGAGYDVHWYQQLPGTAPKLLIYG |
| | | NSNRPNGVPDRFSGSKSGTSASLAITGLQAEDEADYYCQSYDSPTSSSVFGG |
| | | GTKLTVL |
| SEQ ID NO: 358 | DNA VL | cagagcgtgctgacccagccaccaagcgtgagcggtgcaccaggtcagcgcgtgaccattagctgcaccggcagca |
| | | gcagcaacattggcgcaggctatgatgtgcattggtatcagcagctgccaggcaccgcaccgaaactgctgatttatgg |
| | | caacagcaatcgcccaaacggtgtgccggatcgctttagcggcagcaaaagcggcaccagcgccagcctggcgatt |
| | | accggtctgcaagccgaagacgaagccgattattactgccagtcttacgactctccgacttcttcttctgtgtttggcggc |
| | | ggtaccaagctgaccgtgctg |
| SEQ ID NO: 359 | Light Chain | QSVLTQPPSVSGAPGQRVTISCTGSSSNIGAGYDVHWYQQLPGTAPKLLIYG |
| | | NSNRPNGVPDRFSGSKSGTSASLAITGLQAEDEADYYCQSYDSPTSSSVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| SEQ ID NO: 360 | DNA Light Chain | cagagcgtgctgacccagccaccaagcgtgagcggtgcaccaggtcagcgcgtgaccattagctgcaccggcagca |
| | | gcagcaacattggcgcaggctatgatgtgcattggtatcagcagctgccaggcaccgcaccgaaactgctgatttatgg |
| | | caacagcaatcgcccaaacggtgtgccggatcgctttagcggcagcaaaagcggcaccagcgccagcctggcgatt |
| | | accggtctgcaagccgaagacgaagccgattattactgccagtcttacgactctccgacttcttcttctgtgtttggcggc |
| | | ggtaccaagctgaccgtgctgggccagcccaaagccgcccctagcgtgaccctgttccccccaagcagcgaggaac |
| | | tccaggccaacaaggccaccctcgtgtgcctgatcagcgacttctaccctggcgccgtgaccgtggcctggaaggcc |
| | | gatagcagccctgtgaaggccggcgtggaaaccaccacccccagcaagcagagcaacaacaaatacgccgccagc |
| | | agctacctgagcctgacccccgagcagtggaagtcccacagatcctacagctgccaggtcacacacgagggcagca |
| | | ccgtggaaaagaccgtggcccccaccgagtgcagc |
| ZZ05_LALA | | |
| SEQ ID NO: 310 | HCDR1 (Combined) | GYSFTSYWIS |
| SEQ ID NO: 311 | HCDR2 (Combined) | IIYPGTSYTRYSPSFQG |
| SEQ ID NO: 348 | HCDR3 (Combined) | GSLPGLLGFDH |
| SEQ ID NO: 229 | HCDR1 (Kabat) | SYWIS |
| SEQ ID NO: 311 | HCDR2 (Kabat) | IIYPGTSYTRYSPSFQG |
| SEQ ID NO: 348 | HCDR3 (Kabat) | GSLPGLLGFDH |
| SEQ ID NO: 80 | HCDR1 (Chothia) | GYSFTSY |
| SEQ ID NO: 313 | HCDR2 (Chothia) | YPGTSY |
| SEQ ID NO: 348 | HCDR3 (Chothia) | GSLPGLLGFDH |
| SEQ ID NO: 82 | HCDR1 (IMGT) | GYSFTSYW |
| SEQ ID NO: 314 | HCDR2 (IMGT) | IYPGTSYT |
| SEQ ID NO: 349 | HCDR3 (IMGT) | ARGSLPGLLGFDH |
| SEQ ID NO: 350 | VH | EVQLVQSGAEVKKPGESLKISCKGSGYSFTSYWISWVRQMPGKGLEWMGII |
| | | YPGTSYTRYSPSFQGQVTISADKSISTAYLQWSSLKASDTAMYYCARGSLP |
| | | GLLGFDHWGQGTLVTVSS |
| SEQ ID NO: 351 | DNA VH | gaagtgcagctggtgcagagcggtgccgaagtgaaaaaaccgggcgaaagcctgaaaatcagctgcaaaggcagc |
| | | ggctatagctttaccagctattggattagctgggttcgccagatgccgggcaaaggcctggaatggatgggcattatcta |
| | | tccgggcaccagctatacccgctatagcccgagctttcagggccaggttacaattagcgccgacaaaagcatcagcac |
| | | cgcctatctgcaatggagcagcctgaaagccagcgataccgccatgtattattgcgcgcgtggaagcctgcctggtctg |
| | | ctgggttttgatcactggggccagggcaccctggttactgtctcgagc |
| SEQ ID NO: 352 | Heavy Chain | EVQLVQSGAEVKKPGESLKISCKGSGYSFTSYWISWVRQMPGKGLEWMGII |
| | | YPGTSYTRYSPSFQGQVTISADKSISTAYLQWSSLKASDTAMYYCARGSLP |
| | | GLLGFDHWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICNV |
| | | NHKPSNTKVDKRVEPKSCDKTHTCPPCPAPEAAGGPSVFLFPPKPKDTLMIS |
| | | RTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFF |
| | | LYSKLTVDKSRWQQGNVFSCSVMHEALHNHYTQKSLSLSPGK |
| SEQ ID NO: 353 | DNA Heavy Chain | gaagtgcagctggtgcagagcggtgccgaagtgaaaaaaccgggcgaaagcctgaaaatcagctgcaaaggcagc |
| | | ggctatagctttaccagctattggattagctgggttcgccagatgccgggcaaaggcctggaatggatgggcattatcta |
| | | tccgggcaccagctatacccgctatagcccgagctttcagggccaggttacaattagcgccgacaaaagcatcagcac |
| | | cgcctatctgcaatggagcagcctgaaagccagcgataccgccatgtattattgcgcgcgtggaagcctgcctggtctg |
| | | ctgggttttgatcactggggccagggcaccctggttactgtctcgagcgcgtcgaccaaaggccccagcgtgttccctc |
| | | tggcccccagcagcaagagcacctctggcggaacagccgccctgggctgcctggtcaaggactacttccccgagcc |
| | | cgtgaccgtgtcctggaactctggcgccctgaccagcggcgtgcacacctttccagccgtgctccagagcagcggcct |
| | | gtacagcctgagcagcgtcgtgaccgtgcccagcagcagcctgggcacccagacctacatctgcaacgtgaaccaca |
| | | agcccagcaacacaaaggtggacaagcgggggaacccaagagctgcgacaagacccacacctgtcccccctgcc |
| | | ctgcccctgaagcgggggaggcccctccgtgttcctgttccccccaaagcctaaggacaccctgatgatcagccgga |
| | | cccccgaagtgacctgcgtggtggtggacgtgtcccacgaggaccctgaagtgaagtttaattggtacgtggacggcg |
| | | tggaagtgcacaacgccaagaccaagcccagagaggaacagtacaacagcacctaccgggtggtgtccgtgctgac |
| | | cgtgctgcaccaggactggctgaacggcaaagagtacaagtgcaaggtgtccaacaaggccctgcctgcccccatcg |
| | | agaaaaccatcagcaaggccaaaggccagccccgcgagccccaggtgtacacactgccccctagccgggaagaga |
| | | tgaccaagaaccaggtgtccctgacctgcctcgtgaagggcttctaccccagcgacattgccgtggaatgggagagca |
| | | acggccagcccgagaacaactacaagaccaccccccctgtgctggacagcgacggctcattcttcctgtacagcaag |
| | | ctgaccgtggacaagagccggtggcagcagggcaacgtgttcagctgctccgtgatgcacgaggccctgcacaacc |
| | | actacacccagaagtccctgagcctgagccccggcaag |
| SEQ ID NO: 320 | LCDR1 (Combined) | TGSSSNIGAGYDVH |
| SEQ ID NO: 354 | LCDR2 (Combined) | GNSNRPN |
| SEQ ID NO: 361 | LCDR3 (Combined) | QSYGAFPRFVV |
| SEQ ID NO: 320 | LCDR1 (Kabat) | TGSSSNIGAGYDVH |
| SEQ ID NO: 354 | LCDR2 (Kabat) | GNSNRPN |
| SEQ ID NO: 361 | LCDR3 (Kabat) | QSYGAFPRFVV |
| SEQ ID NO: 323 | LCDR1 (Chothia) | SSSNIGAGYD |
| SEQ ID NO: 324 | LCDR2 (Chothia) | GNS |
| SEQ ID NO: 362 | LCDR3 (Chothia) | YGAFPRFV |
| SEQ ID NO: 326 | LCDR1 (IMGT) | SSNIGAGYD |
| SEQ ID NO: 324 | LCDR2 (IMGT) | GNS |
| SEQ ID NO: 361 | LCDR3 (IMGT) | QSYGAFPRFVV |
| SEQ ID NO: 363 | VL | QSVLTQPPSVSGAPGQRVTISCTGSSSNIGAGYDVHWYQQLPGTAPKLLIYG |
| | | NSNRPNGVPDRFSGSKSGTSASLAITGLQAEDEADYYCQSYGAFPRFVVFG |
| | | GGTKLTVL |
| SEQ ID NO: 364 | DNA VL | cagagcgtgctgacccagccaccaagcgtgagcggtgcaccaggtcagcgcgtgaccattagctgcaccggcagca |
| | | gcagcaacattggcgcaggctatgatgtgcattggtatcagcagctgccaggcaccgcaccgaaactgctgatttatgg |
| | | caacagcaatcgcccaaacggtgtgccggatcgctttagcggcagcaaaagcggcaccagcgccagcctggcgatt |
| | | accggtctgcaagccgaagacgaagccgattattactgccaatcctatggtgccttccctcgtttcgttgtttttggcggcg |
| | | gtaccaagctgaccgtgctg |
| SEQ ID NO: 365 | Light Chain | QSVLTQPPSVSGAPGQRVTISCTGSSSNIGAGYDVHWYQQLPGTAPKLLIYG |
| | | NSNRPNGVPDRFSGSKSGTSASLAITGLQAEDEADYYCQSYGAFPRFVVFG |
| | | GGTKLTVLGQPKAAPSVTLFPPSSEELQANKATLVCLISDFYPGAVTVAWK |
| | | ADSSPVKAGVETTTPSKQSNNKYAASSYLSLTPEQWKSHRSYSCQVTHEGS |
| | | TVEKTVAPTECS |
| SEQ ID NO: 366 | DNA Light Chain | cagagcgtgctgacccagccaccaagcgtgagcggtgcaccaggtcagcgcgtgaccattagctgcaccggcagca |
| | | gcagcaacattggcgcaggctatgatgtgcattggtatcagcagctgccaggcaccgcaccgaaactgctgatttatgg |
| | | caacagcaatcgcccaaacggtgtgccggatcgctttagcggcagcaaaagcggcaccagcgccagcctggcgatt |
| | | accggtctgcaagccgaagacgaagccgattattactgccaatcctatggtgccttccctcgtttcgttgtttttggcggcg |
| | | gtaccaagctgaccgtgctgggccagcccaaagccgcccctagcgtgaccctgttccccccaagcagcgaggaactc |
| | | caggccaacaaggccaccctcgtgtgcctgatcagcgacttctaccctggcgccgtgaccgtggcctggaaggccga |
| | | tagcagccctgtgaaggccggcgtggaaaccaccacccccagcaagcagagcaacacaaatacgccgccagcag |
| | | ctacctgagcctgacccccgagcagtggaagtcccacagatcctacagctgccaggtcacacacgagggcagcacc |
| | | gtggaaaagaccgtggcccccaccgagtgcagc |
| ZZ12_LALA | | |
| SEQ ID NO: 367 | HCDR1 (Combined) | GFSFSKYYLN |
| SEQ ID NO: 368 | HCDR2 (Combined) | SIHQQAHEKKYVESVKG |
| SEQ ID NO: 228 | HCDR3 (Combined) | SLRRRSTEHAGFDV |
| SEQ ID NO: 369 | HCDR1 (Kabat) | KYYLN |
| SEQ ID NO: 368 | HCDR2 (Kabat) | SIHQQAHEKKYVESVKG |
| SEQ ID NO: 228 | HCDR3 (Kabat) | SLRRRSTEHAGFDV |
| SEQ ID NO: 370 | HCDR1 (Chothia) | GFSFSKY |
| SEQ ID NO: 371 | HCDR2 (Chothia) | HQQAHE |
| SEQ ID NO: 228 | HCDR3 (Chothia) | SLRRRSTEHAGFDV |
| SEQ ID NO: 372 | HCDR1 (IMGT) | GFSFSKYY |
| SEQ ID NO: 373 | HCDR2 (IMGT) | IHQQAHEK |
| SEQ ID NO: 232 | HCDR3 (IMGT) | ARSLRRRSTEHAGFDV |
| SEQ ID NO: 374 | VH | EVQLVESGGGLVQPGGSLRLSCAASGFSFSKYYLNWVRQAPGKGLEWVAS |
| | | IHQQAHEKKYVESVKGRFTISRDNAKNSLYLQMNSLRAEDTAVYYCARSL |
| | | RRRSTEHAGFDVWGQGTLVTVSS |
| SEQ ID NO: 375 | DNA VH | gaagtgcagctggtggaaagcggcggtggcctggtgcagccaggtggtagcctgcgcctgagctgcgccgccagc |
| | | ggctttagcttcagcaaatattacttgaactgggttcgccaggccccaggcaaaggcctggaatgggtggccagcattc |
| | | accagcaagcacacgagaaaaaatacgtggagtccgtgaaaggccgctttaccattagccgcgataacgccaaaaac |
| | | agcctgtatctgcaaatgaacagcctgcgggccgaagataccgccgtgtattattgcgcgcgtagcctgcgtcgtcgta |
| | | gcactgagcacgcaggattcgacgtttggggccagggcaccctggttactgtctcgagc |
| SEQ ID NO: 376 | Heavy Chain | EVQLVESGGGLVQPGGSLRLSCAASGFSFSKYYLNWVRQAPGKGLEWVAS |
| | | IHQQAHEKKYVESVKGRFTISRDNAKNSLYLQMNSLRAEDTAVYYCARSL |
| | | RRRSTEHAGFDVWGQGTLVTVSSASTKGPSVFPLAPSSKSTSGGTAALGCL |
| | | VKDYFPEPVTVSWNSGALTSGVHTFPAVLQSSGLYSLSSVVTVPSSSLGTQT |
| | | YICNVNHKPSNTKVDKRVEPKSCDKTHTCPPCPAPEAAGGPSVFLFPPKPK |
| | | DTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| SEQ ID NO: 377 | DNA Heavy Chain | gaagtgcagctggtggaaagcggcggtggcctggtgcagccaggtggtagcctgcgcctgagctgcgccgccagc |
| | | ggctttagcttcagcaaatattacttgaactgggttcgccaggccccaggcaaaggcctggaatgggtggccagcattc |
| | | accagcaagcacacgagaaaaaatacgtggagtccgtgaaaggccgctttaccattagccgcgataacgccaaaaac |
| | | agcctgtatctgcaaatgaacagcctgcgggccgaagataccgccgtgtattattgcgcgcgtagcctgcgtcgtcgta |
| | | gcactgagcacgcaggattcgacgtttggggccagggcaccctggttactgtctcgagcgcgtcgaccaaaggcccc |
| | | agcgtgttccctctggcccccagcagcaagagcacctctggcggaacagccgccctgggctgcctggtcaaggacta |
| | | cttccccgagcccgtgaccgtgtcctggaactctggcgccctgaccagcggcgtgcacacctttccagccgtgctcca |
| | | gagcagcggcctgtacagcctgagcagcgtcgtgaccgtgcccagcagcagcctgggcacccagacctacatctgc |
| | | aacgtgaaccacaagcccagcaacacaaaggtggacaagcgggggaacccaagagctgcgacaagacccacac |
| | | ctgtcccccctgccctgcccctgaagcgggggaggcccctccgtgttcctgttccccccaaagcctaaggacaccct |
| | | gatgatcagccggacccccgaagtgacctgcgtggtggtggacgtgtcccacgaggaccctgaagtgaagtttaattg |
| | | gtacgtggacggcgtggaagtgcacaacgccaagaccaagcccagagaggaacagtacaacagcacctaccgggt |
| | | ggtgtccgtgctgaccgtgctgcaccaggactggctgaacggcaaagagtacaagtgcaaggtgtccaacaaggccc |
| | | tgcctgcccccatcgagaaaaccatcagcaaggccaaaggccagccccgcgagccccaggtgtacacactgccccc |
| | | tagccgggaagagatgaccaagaaccaggtgtccctgacctgcctcgtgaagggcttctaccccagcgacattgccgt |
| | | ggaatgggagagcaacggccagcccgagaacaactacaagaccaccccccctgtgctggacagcgacggctcattc |
| | | ttcctgtacagcaagctgaccgtggacaagagccggtggcagcagggcaacgtgttcagctgctccgtgatgcacga |
| | | ggccctgcacaaccactacacccagaagtccctgagcctgagccccggcaag |
| SEQ ID NO: 237 | LCDR1 (Combined) | RASQGISNYLA |
| SEQ ID NO: 238 | LCDR2 (Combined) | AASTLQS |
| SEQ ID NO: 239 | LCDR3 (Combined) | QQADKFPYT |
| SEQ ID NO: 237 | LCDR1 (Kabat) | RASQGISNYLA |
| SEQ ID NO: 238 | LCDR2 (Kabat) | AASTLQS |
| SEQ ID NO: 239 | LCDR3 (Kabat) | QQADKFPYT |
| SEQ ID NO: 240 | LCDR1 (Chothia) | SQGISNY |
| SEQ ID NO: 241 | LCDR2 (Chothia) | AAS |
| SEQ ID NO: 242 | LCDR3 (Chothia) | ADKFPY |
| SEQ ID NO: 243 | LCDR1 (IMGT) | QGISNY |
| SEQ ID NO: 241 | LCDR2 (IMGT) | AAS |
| SEQ ID NO: 239 | LCDR3 (IMGT) | QQADKFPYT |
| SEQ ID NO: 244 | VL | DIQMTQSPSSLSASVGDRVTITCRASQGISNYLAWYQQKPGKVPKLLIYAAS |
| | | TLQSGVPSRFSGSGSGTDFTLTISSLQPEDVATYYCQQADKFPYTFGQGTKV |
| | | EIK |
| SEQ ID NO: 245 | DNA VL | gatattcagatgacccagagcccgagcagcctgagcgcaagcgtgggcgatcgcgtgaccattacctgccgcgcca |
| | | gccagggcattagcaactatctggcctggtatcagcagaaaccgggcaaagtgccgaaactgctgatctatgccgcca |
| | | gcaccctgcaaagcggcgtgccaagtcgctttagcggcagcggtagcggcaccgatttcaccctgaccattagcagc |
| | | ctgcaaccggaagacgtggcgacctattattgccagcaggctgacaaattcccgtacaccttcggccagggtaccaaa |
| | | gtggaaatcaag |
| SEQ ID NO: 246 | Light Chain | DIQMTQSPSSLSASVGDRVTITCRASQGISNYLAWYQQKPGKVPKLLIYAAS |
| | | TLQSGVPSRFSGSGSGTDFTLTISSLQPEDVATYYCQQADKFPYTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 247 | DNA Light Chain | gatattcagatgacccagagcccgagcagcctgagcgcaagcgtgggcgatcgcgtgaccattacctgccgcgcca |
| | | gccagggcattagcaactatctggcctggtatcagcagaaaccgggcaaagtgccgaaactgctgatctatgccgcca |
| | | gcaccctgcaaagcggcgtgccaagtcgctttagcggcagcggtagcggcaccgatttcaccctgaccattagcagc |
| | | ctgcaaccggaagacgtggcgacctattattgccagcaggctgacaaattcccgtacaccttcggccagggtaccaaa |
| | | gtggaaatcaagcggaccgtggccgctccctccgtgttcatcttcccacccagcgacgagcagctgaagtccggcaca |
| | | gccagcgtcgtgtgcctgctgaacaacttctacccccgcgaggccaaagtgcagtggaaggtggacaacgccctcca |
| | | gagcggcaacagccaggaaagcgtcaccgagcaggacagcaaggactccacctacagcctgagcagcaccctgac |
| | | cctgagcaaggccgactacgagaagcacaaggtgtacgcctgcgaagtgacccaccagggcctgtccagccccgtg |
| | | accaagagcttcaaccggggcgagtgt |
| ZZ13_LALA | | |
| SEQ ID NO: 378 | HCDR1 (Combined) | GFTFSRYYIN |
| SEQ ID NO: 379 | HCDR2 (Combined) | SIHQHGLETRYVESVKG |
| SEQ ID NO: 228 | HCDR3 (Combined) | SLRRRSTEHAGFDV |
| SEQ ID NO: 380 | HCDR1 (Kabat) | RYYIN |
| SEQ ID NO: 379 | HCDR2 (Kabat) | SIHQHGLETRYVESVKG |
| SEQ ID NO: 228 | HCDR3 (Kabat) | SLRRRSTEHAGFDV |
| SEQ ID NO: 381 | HCDR1 (Chothia) | GFTFSRY |
| SEQ ID NO: 382 | HCDR2 (Chothia) | HQHGLE |
| SEQ ID NO: 228 | HCDR3 (Chothia) | SLRRRSTEHAGFDV |
| SEQ ID NO: 383 | HCDR1 (IMGT) | GFTFSRYY |
| SEQ ID NO: 384 | HCDR2 (IMGT) | IHQHGLET |
| SEQ ID NO: 232 | HCDR3 (IMGT) | ARSLRRRSTEHAGFDV |
| SEQ ID NO: 385 | VH | EVQLVESGGGLVQPGGSLRLSCAASGFTFSRYYINWVRQAPGKGLEWVASI |
| | | HQHGLETRYVESVKGRFTISRDNAKNSLYLQMNSLRAEDTAVYYCARSLR |
| | | RRSTEHAGFDVWGQGTLVTVSS |
| SEQ ID NO: 386 | DNA VH | gaagtgcagctggtggaaagcggcggtggcctggtgcagccaggtggtagcctgcgcctgagctgcgccgccagc |
| | | gggtttactttttccagatattacattaattgggttcgccaggccccaggcaaaggcctggaatgggggcgagcatcca |
| | | ccagcacggcctggagaccagatatgtggaatctgtcaaagggcgctttaccattagccgcgataacgccaaaaacag |
| | | cctgtatctgcaaatgaacagcctgcgggccgaagataccgccgtgtattattgcgcgcgtagcctgcgtcgtcgtagc |
| | | actgagcacgcaggattcgacgtttggggccagggcaccctggttactgtctcgagc |
| SEQ ID NO: 387 | Heavy Chain | EVQLVESGGGLVQPGGSLRLSCAASGFTFSRYYINWVRQAPGKGLEWVASI |
| | | HQHGLETRYVESVKGRFTISRDNAKNSLYLQMNSLRAEDTAVYYCARSLR |
| | | RRSTEHAGFDVWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTY |
| | | ICNVNHKPSNTKVDKRVEPKSCDKTHTCPPCPAPEAAGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 388 | DNA Heavy Chain | gaagtgcagctggtggaaagcggcggtggcctggtgcagccaggtggtagcctgcgcctgagctgcgccgccagc |
| | | gggtttactttttccagatattacattaattgggttcgccaggccccaggcaaaggcctggaatgggtggcgagcatcca |
| | | ccagcacggcctggagaccagatatgtggaatctgtcaaagggcgctttaccattagccgcgataacgccaaaaacag |
| | | cctgtatctgcaaatgaacagcctgcgggccgaagataccgccgtgtattattgcgcgcgtagcctgcgtcgtcgtagc |
| | | actgagcacgcaggattcgacgtttggggccagggcaccctggttactgtctcgagcgcgtcgaccaaaggccccag |
| | | cgtgttccctctggcccccagcagcaagagcacctctggcggaacagccgccctgggctgcctggtcaaggactactt |
| | | ccccgagcccgtgaccgtgtcctggaactctggcgccctgaccagcggcgtgcacacctttccagccgtgctccaga |
| | | gcagcggcctgtacagcctgagcagcgtcgtgaccgtgcccagcagcagcctgggcacccagacctacatctgcaa |
| | | cgtgaaccacaagcccagcaacacaaaggtggacaagcgggtggaacccaagagctgcgacaagacccacacctg |
| | | tcccccctgccctgcccctgaagcgggggaggcccctccgtgttcctgttccccccaaagcctaaggacaccctgat |
| | | gatcagccggacccccgaagtgacctgcgtggtggtggacgtgtcccacgaggaccctgaagtgaagtttaattggta |
| | | cgtggacggcgtggaagtgcacaacgccaagaccaagcccagagaggaacagtacaacagcacctaccgggtggt |
| | | gtccgtgctgaccgtgctgcaccaggactggctgaacggcaaagagtacaagtgcaaggtgtccaacaaggccctgc |
| | | ctgcccccatcgagaaaaccatcagcaaggccaaaggccagccccgcgagccccaggtgtacacactgccccctag |
| | | ccgggaagagatgaccaagaaccaggtgtccctgacctgcctcgtgaagggcttctaccccagcgacattgccgtgg |
| | | aatgggagagcaacggccagcccgagaacaactacaagaccaccccccctgtgctggacagcgacggctcattcttc |
| | | ctgtacagcaagctgaccgtggacaagagccggtggcagcagggcaacgtgttcagctgctccgtgatgcacgagg |
| | | ccctgcacaaccactacacccagaagtccctgagcctgagccccggcaag |
| SEQ ID NO: 237 | LCDR1 (Combined) | RASQGISNYLA |
| SEQ ID NO: 238 | LCDR2 (Combined) | AASTLQS |
| SEQ ID NO: 239 | LCDR3 (Combined) | QQADKFPYT |
| SEQ ID NO: 237 | LCDR1 (Kabat) | RASQGISNYLA |
| SEQ ID NO: 238 | LCDR2 (Kabat) | AASTLQS |
| SEQ ID NO: 239 | LCDR3 (Kabat) | QQADKFPYT |
| SEQ ID NO: 240 | LCDR1 (Chothia) | SQGISNY |
| SEQ ID NO: 241 | LCDR2 (Chothia) | AAS |
| SEQ ID NO: 242 | LCDR3 (Chothia) | ADKFPY |
| SEQ ID NO: 243 | LCDR1 (IMGT) | QGISNY |
| SEQ ID NO: 241 | LCDR2 (IMGT) | AAS |
| SEQ ID NO: 239 | LCDR3 (IMGT) | QQADKFPYT |
| SEQ ID NO: 244 | VL | DIQMTQSPSSLSASVGDRVTITCRASQGISNYLAWYQQKPGKVPKLLIYAAS |
| | | TLQSGVPSRFSGSGSGTDFTLTISSLQPEDVATYYCQQADKFPYTFGQGTKV |
| | | EIK |
| SEQ ID NO: 245 | DNA VL | gatattcagatgacccagagcccgagcagcctgagcgcaagcgtgggcgatcgcgtgaccattacctgccgcgcca |
| | | gccagggcattagcaactatctggcctggtatcagcagaaaccgggcaaagtgccgaaactgctgatctatgccgcca |
| | | gcaccctgcaaagcggcgtgccaagtcgctttagcggcagcggtagcggcaccgatttcaccctgaccattagcagc |
| | | ctgcaaccggaagacgtggcgacctattattgccagcaggctgacaaattcccgtacaccttcggccagggtaccaaa |
| | | gtggaaatcaag |
| SEQ ID NO: 246 | Light Chain | DIQMTQSPSSLSASVGDRVTITCRASQGISNYLAWYQQKPGKVPKLLIYAAS |
| | | TLQSGVPSRFSGSGSGTDFTLTISSLQPEDVATYYCQQADKFPYTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 247 | DNA Light Chain | gatattcagatgacccagagcccgagcagcctgagcgcaagcgtgggcgatcgcgtgaccattacctgccgcgcca |
| | | gccagggcattagcaactatctggcctggtatcagcagaaaccgggcaaagtgccgaaactgctgatctatgccgcca |
| | | gcaccctgcaaagcggcgtgccaagtcgctttagcggcagcggtagcggcaccgatttcaccctgaccattagcagc |
| | | ctgcaaccggaagacgtggcgacctattattgccagcaggctgacaaattcccgtacaccttcggccagggtaccaaa |
| | | gtggaaatcaagcggaccgtggccgctccctccgtgttcatcttcccacccagcgacgagcagctgaagtccggcaca |
| | | gccagcgtcgtgtgcctgctgaacaacttctacccccgcgaggccaaagtgcagtggaaggtggacaacgccctcca |
| | | gagcggcaacagccaggaaagcgtcaccgagcaggacagcaaggactccacctacagcctgagcagcaccctgac |
| | | cctgagcaaggccgactacgagaagcacaaggtgtacgcctgcgaagtgacccaccagggcctgtccagccccgtg |
| | | accaagagcttcaaccggggcgagtgt |
| ZZ14_LALA | | |
| SEQ ID NO: 270 | HCDR1 (Combined) | GFTFSSYAIS |
| SEQ ID NO: 389 | HCDR2 (Combined) | SISSHGYYTRYAESVKG |
| SEQ ID NO: 331 | HCDR3 (Combined) | PYLGDRRSYGFDH |
| SEQ ID NO: 273 | HCDR1 (Kabat) | SYAIS |
| SEQ ID NO: 389 | HCDR2 (Kabat) | SISSHGYYTRYAESVKG |
| SEQ ID NO: 331 | HCDR3 (Kabat) | PYLGDRRSYGFDH |
| SEQ ID NO: 32 | HCDR1 (Chothia) | GFTFSSY |
| SEQ ID NO: 390 | HCDR2 (Chothia) | SSHGYY |
| SEQ ID NO: 331 | HCDR3 (Chothia) | PYLGDRRSYGFDH |
| SEQ ID NO: 275 | HCDR1 (IMGT) | GFTFSSYA |
| SEQ ID NO: 391 | HCDR2 (IMGT) | ISSHGYYT |
| SEQ ID NO: 332 | HCDR3 (IMGT) | ARPYLGDRRSYGFDH |
| SEQ ID NO: 392 | VH | EVQLLESGGGLVQPGGSLRLSCAASGFTFSSYAISWVRQAPGKGLEWVSSIS |
| | | SHGYYTRYAESVKGRFTISRDNSKNTLYLQMNSLRAEDTAVYYCARPYLG |
| | | DRRSYGFDHWGQGTLVTVSS |
| SEQ ID NO: 393 | DNA VH | gaagtgcagctgctggaaagcggtggcggtctggtgcagccaggtggtagcctgcgcctgagctgtgccgcaagcg |
| | | ggtttacattttccagctatgctatcagctgggtgcgccaagcaccaggcaaaggcctggaatgggtgagcagcattag |
| | | ctcacatggatattacacccggtatgccgagtccgtgaaaggtcgctttaccattagtcgcgataacagcaaaaacaccc |
| | | tgtatctgcaaatgaacagcctgcgggcagaagataccgcagtttattattgcgcgcgaccttatctgggtgaccgtcgt |
| | | agctatggtttcgaccactggggccagggcaccctggttactgtctcgagc |
| SEQ ID NO: 394 | Heavy Chain | EVQLLESGGGLVQPGGSLRLSCAASGFTFSSYAISWVRQAPGKGLEWVSSIS |
| | | SHGYYTRYAESVKGRFTISRDNSKNTLYLQMNSLRAEDTAVYYCARPYLG |
| | | DRRSYGFDHWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQTYIC |
| | | NVNHKPSNTKVDKRVEPKSCDKTHTCPPCPAPEAAGGPSVFLFPPKPKDTL |
| | | MISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTLP |
| | | PSREEMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| SEQ ID NO: 395 | DNA Heavy Chain | gaagtgcagctgctggaaagcggtggcggtctggtgcagccaggtggtagcctgcgcctgagctgtgccgcaagcg |
| | | ggtttacattttccagctatgctatcagctgggtgcgccaagcaccaggcaaaggcctggaatgggtgagcagcattag |
| | | ctcacatggatattacacccggtatgccgagtccgtgaaaggtcgctttaccattagtcgcgataacagcaaaaacaccc |
| | | tgtatctgcaaatgaacagcctgcgggcagaagataccgcagtttattattgcgcgcgaccttatctgggtgaccgtcgt |
| | | agctatggtttcgaccactggggccagggcaccctggttactgtctcgagcgcgtcgaccaaaggccccagcgtgttc |
| | | cctctggcccccagcagcaagagcacctctggcggaacagccgccctgggctgcctggtcaaggactacttccccga |
| | | gcccgtgaccgtgtcctggaactctggcgccctgaccagcggcgtgcacacctttccagccgtgctccagagcagcg |
| | | gcctgtacagcctgagcagcgtcgtgaccgtgcccagcagcagcctgggcacccagacctacatctgcaacgtgaac |
| | | cacaagcccagcaacacaaaggtggacaagcgggtggaacccaagagctgcgacaagacccacacctgtcccccc |
| | | tgccctgcccctgaagcgggggaggcccctccgtgttcctgttccccccaaagcctaaggacaccctgatgatcagc |
| | | cggacccccgaagtgacctgcgtggtggtggacgtgtcccacgaggaccctgaagtgaagtttaattggtacgtggac |
| | | ggcgtggaagtgcacaacgccaagaccaagcccagagaggaacagtacaacagcacctaccgggtggtgtccgtg |
| | | ctgaccgtgctgcaccaggactggctgaacggcaaagagtacaagtgcaaggtgtccaacaaggccctgcctgcccc |
| | | catcgagaaaaccatcagcaaggccaaaggccagccccgcgagccccaggtgtacacactgccccctagccggga |
| | | agagatgaccaagaaccaggtgtccctgacctgcctcgtgaagggcttctaccccagcgacattgccgtggaatggga |
| | | gagcaacggccagcccgagaacaactacaagaccaccccccctgtgctggacagcgacggctcattcttcctgtaca |
| | | gcaagctgaccgtggacaagagccggtggcagcagggcaacgtgttcagctgctccgtgatgcacgaggccctgca |
| | | caaccactacacccagaagtccctgagcctgagccccggcaag |
| SEQ ID NO: 337 | LCDR1 (Combined) | TGTSSDVGSYNLVS |
| SEQ ID NO: 338 | LCDR2 (Combined) | EGSKRPS |
| SEQ ID NO: 339 | LCDR3 (Combined) | SSYGFHIVVVV |
| SEQ ID NO: 337 | LCDR1 (Kabat) | TGTSSDVGSYNLVS |
| SEQ ID NO: 338 | LCDR2 (Kabat) | EGSKRPS |
| SEQ ID NO: 339 | LCDR3 (Kabat) | SSYGFHIVVVV |
| SEQ ID NO: 340 | LCDR1 (Chothia) | TSSDVGSYNL |
| SEQ ID NO: 341 | LCDR2 (Chothia) | EGS |
| SEQ ID NO: 342 | LCDR3 (Chothia) | YGFHIVVV |
| SEQ ID NO: 343 | LCDR1 (IMGT) | SSDVGSYNL |
| SEQ ID NO: 341 | LCDR2 (IMGT) | EGS |
| SEQ ID NO: 339 | LCDR3 (IMGT) | SSYGFHIVVVV |
| SEQ ID NO: 344 | VL | QSALTQPASVSGSPGQSITISCTGTSSDVGSYNLVSWYQQHPGKAPKLMIYE |
| | | GSKRPSGVSNRFSGSKSGNTASLTISGLQAEDEADYYCSSYGFHIVVVVFGG |
| | | GTKLTVL |
| SEQ ID NO: 345 | DNA VL | cagagcgccctgacccagccagccagcgttagcggtagcccaggccagagcattaccattagctgcaccggcacca |
| | | gcagcgacgtgggcagctataacctggttagctggtatcagcagcatccgggcaaagccccgaaactgatgatctatg |
| | | aaggcagcaaacgcccgagcggcgttagcaaccgctttagtggcagcaaaagcggcaacaccgccagcctgaccat |
| | | tagcggcctgcaagccgaagacgaagccgattattactgctcctcttacggtttccatatcgttgttgttgtgtttggcggc |
| | | ggtaccaagctgaccgtgctg |
| SEQ ID NO: 346 | Light Chain | QSALTQPASVSGSPGQSITISCTGTSSDVGSYNLVSWYQQHPGKAPKLMIYE |
| | | GSKRPSGVSNRFSGSKSGNTASLTISGLQAEDEADYYCSSYGFHIVVVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| SEQ ID NO: 347 | DNA Light Chain | cagagcgccctgacccagccagccagcgttagcggtagcccaggccagagcattaccattagctgcaccggcacca |
| | | gcagcgacgtgggcagctataacctggttagctggtatcagcagcatccgggcaaagccccgaaactgatgatctatg |
| | | aaggcagcaaacgcccgagcggcgttagcaaccgctttagtggcagcaaaagcggcaacaccgccagcctgaccat |
| | | tagcggcctgcaagccgaagacgaagccgattattactgctcctcttacggtttccatatcgttgttgttgtgtttggcggc |
| | | ggtaccaagctgaccgtgctgggccagcccaaagccgcccctagcgtgaccctgttccccccaagcagcgaggaac |
| | | tccaggccaacaaggccaccctcgtgtgcctgatcagcgacttctaccctggcgccgtgaccgtggcctggaaggcc |
| | | gatagcagccctgtgaaggccggcgtggaaaccaccacccccagcaagcagagcaacaacaaatacgccgccagc |
| | | agctacctgagcctgacccccgagcagtggaagtcccacagatcctacagctgccaggtcacacacgagggcagca |
| | | ccgtggaaaagaccgtggcccccaccgagtgcagc |
| ZZ15_LALA | | |
| SEQ ID NO: 396 | HCDR1 (Combined) | GFTFASYAIT |
| SEQ ID NO: 397 | HCDR2 (Combined) | TISGSGVYTYYAESVKG |
| SEQ ID NO: 331 | HCDR3 (Combined) | PYLGDRRSYGFDH |
| SEQ ID NO: 398 | HCDR1 (Kabat) | SYAIT |
| SEQ ID NO: 397 | HCDR2 (Kabat) | TISGSGVYTYYAESVKG |
| SEQ ID NO: 331 | HCDR3 (Kabat) | PYLGDRRSYGFDH |
| SEQ ID NO: 399 | HCDR1 (Chothia) | GFTFASY |
| SEQ ID NO: 400 | HCDR2 (Chothia) | SGSGVY |
| SEQ ID NO: 331 | HCDR3 (Chothia) | PYLGDRRSYGFDH |
| SEQ ID NO: 401 | HCDR1 (IMGT) | GFTFASYA |
| SEQ ID NO: 402 | HCDR2 (IMGT) | ISGSGVYT |
| SEQ ID NO: 332 | HCDR3 (IMGT) | ARPYLGDRRSYGFDH |
| SEQ ID NO: 403 | VH | EVQLLESGGGLVQPGGSLRLSCAASGFTFASYAITWVRQAPGKGLEWVSTI |
| | | SGSGVYTYYAESVKGRFTISRDNSKNTLYLQMNSLRAEDTAVYYCARPYL |
| | | GDRRSYGFDHWGQGTLVTVSS |
| SEQ ID NO: 404 | DNA VH | gaagtgcagctgctggaaagcggtggcggtctggtgcagccaggtggtagcctgcgcctgagctgtgccgcaagcg |
| | | ggttcacattcgcatcctatgcaattacttgggtgcgccaagcaccaggcaaaggcctggaatgggtgagcaccatttc |
| | | cgggtccggtgtgtacacctattacgccgagtccgtcaaaggccgctttaccattagtcgcgataacagcaaaaacacc |
| | | ctgtatctgcaaatgaacagcctgcgggcagaagataccgcagtttattattgcgcgcgaccttatctgggtgaccgtcg |
| | | tagctatggtttcgaccactggggccagggcaccctggttactgtctcgagc |
| SEQ ID NO: 405 | Heavy Chain | EVQLLESGGGLVQPGGSLRLSCAASGFTFASYAITWVRQAPGKGLEWVSTI |
| | | SGSGVYTYYAESVKGRFTISRDNSKNTLYLQMNSLRAEDTAVYYCARPYL |
| | | GDRRSYGFDHWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKRVEPKSCDKTHTCPPCPAPEAAGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 406 | DNA Heavy Chain | gaagtgcagctgctggaaagcggtggcggtctggtgcagccaggtggtagcctgcgcctgagctgtgccgcaagcg |
| | | ggttcacattcgcatcctatgcaattacttgggtgcgccaagcaccaggcaaaggcctggaatgggtgagcaccatttc |
| | | cgggtccggtgtgtacacctattacgccgagtccgtcaaaggccgctttaccattagtcgcgataacagcaaaaacacc |
| | | ctgtatctgcaaatgaacagcctgcgggcagaagataccgcagtttattattgcgcgcgaccttatctgggtgaccgtcg |
| | | tagctatggtttcgaccactggggccagggcaccctggttactgtctcgagcgcgtcgaccaaaggccccagcgtgttc |
| | | cctctggcccccagcagcaagagcacctctggcggaacagccgccctgggctgcctggtcaaggactacttccccga |
| | | gcccgtgaccgtgtcctggaactctggcgccctgaccagcggcgtgcacacctttccagccgtgctccagagcagcg |
| | | gcctgtacagcctgagcagcgtcgtgaccgtgcccagcagcagcctgggcacccagacctacatctgcaacgtgaac |
| | | cacaagcccagcaacacaaaggtggacaagcgggtggaacccaagagctgcgacaagacccacacctgtcccccc |
| | | tgccctgcccctgaagcgggggaggcccctccgtgttcctgttccccccaaagcctaaggacaccctgatgatcagc |
| | | cggacccccgaagtgacctgcgtggtggtggacgtgtcccacgaggaccctgaagtgaagtttaattggtacgtggac |
| | | ggcgtggaagtgcacaacgccaagaccaagcccagagaggaacagtacaacagcacctaccgggtggtgtccgtg |
| | | ctgaccgtgctgcaccaggactggctgaacggcaaagagtacaagtgcaaggtgtccaacaaggccctgcctgcccc |
| | | catcgagaaaaccatcagcaaggccaaaggccagccccgcgagccccaggtgtacacactgccccctagccggga |
| | | agagatgaccaagaaccaggtgtccctgacctgcctcgtgaagggcttctaccccagcgacattgccgtggaatggga |
| | | gagcaacggccagcccgagaacaactacaagaccaccccccctgtgctggacagcgacggctcattcttcctgtaca |
| | | gcaagctgaccgtggacaagagccggtggcagcagggcaacgtgttcagctgctccgtgatgcacgaggccctgca |
| | | caaccactacacccagaagtccctgagcctgagccccggcaag |
| SEQ ID NO: 337 | LCDR1 (Combined) | TGTSSDVGSYNLVS |
| SEQ ID NO: 338 | LCDR2 (Combined) | EGSKRPS |
| SEQ ID NO: 339 | LCDR3 (Combined) | SSYGFHIVVVV |
| SEQ ID NO: 337 | LCDR1 (Kabat) | TGTSSDVGSYNLVS |
| SEQ ID NO: 338 | LCDR2 (Kabat) | EGSKRPS |
| SEQ ID NO: 339 | LCDR3 (Kabat) | SSYGFHIVVVV |
| SEQ ID NO: 340 | LCDR1 (Chothia) | TSSDVGSYNL |
| SEQ ID NO: 341 | LCDR2 (Chothia) | EGS |
| SEQ ID NO: 342 | LCDR3 (Chothia) | YGFHIVVV |
| SEQ ID NO: 343 | LCDR1 (IMGT) | SSDVGSYNL |
| SEQ ID NO: 341 | LCDR2 (IMGT) | EGS |
| SEQ ID NO: 339 | LCDR3 (IMGT) | SSYGFHIVVVV |
| SEQ ID NO: 344 | VL | QSALTQPASVSGSPGQSITISCTGTSSDVGSYNLVSWYQQHPGKAPKLMIYE |
| | | GSKRPSGVSNRFSGSKSGNTASLTISGLQAEDEADYYCSSYGFHIVVVVFGG |
| | | GTKLTVL |
| SEQ ID NO: 345 | DNA VL | cagagcgccctgacccagccagccagcgttagcggtagcccaggccagagcattaccattagctgcaccggcacca |
| | | gcagcgacgtgggcagctataacctggttagctggtatcagcagcatccgggcaaagccccgaaactgatgatctatg |
| | | aaggcagcaaacgcccgagcggcgttagcaaccgctttagtggcagcaaaagcggcaacaccgccagcctgaccat |
| | | tagcggcctgcaagccgaagacgaagccgattattactgctcctcttacggtttccatatcgttgttgttgtgtttggcggc |
| | | ggtaccaagctgaccgtgctg |
| SEQ ID NO: 346 | Light Chain | QSALTQPASVSGSPGQSITISCTGTSSDVGSYNLVSWYQQHPGKAPKLMIYE |
| | | GSKRPSGVSNRFSGSKSGNTASLTISGLQAEDEADYYCSSYGFHIVVVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| SEQ ID NO: 347 | DNA Light Chain | cagagcgccctgacccagccagccagcgttagcggtagcccaggccagagcattaccattagctgcaccggcacca |
| | | gcagcgacgtgggcagctataacctggttagctggtatcagcagcatccgggcaaagccccgaaactgatgatctatg |
| | | aaggcagcaaacgcccgagcggcgttagcaaccgctttagtggcagcaaaagcggcaacaccgccagcctgaccat |
| | | tagcggcctgcaagccgaagacgaagccgattattactgctcctcttacggtttccatatcgttgttgttgtgtttggggc |
| | | ggtaccaagctgaccgtgctgggccagcccaaagccgcccctagcgtgaccctgttccccccaagcagcgaggaac |
| | | tccaggccaacaaggccaccctcgtgtgcctgatcagcgacttctaccctggcgccgtgaccgtggcctggaaggcc |
| | | gatagcagccctgtgaaggccggcgtggaaaccaccacccccagcaagcagagcaacaacaaatacgccgccagc |
| | | agctacctgagcctgacccccgagcagtggaagtcccacagatcctacagctgccaggtcacacacgagggcagca |
| | | ccgtggaaaagaccgtggcccccaccgagtgcagc |
| ZZ16_LALA | | |
| SEQ ID NO: 407 | HCDR1 (Combined) | GFTFGTYAMT |
| SEQ ID NO: 408 | HCDR2 (Combined) | SISASGYYANYAGSVKG |
| SEQ ID NO: 331 | HCDR3 (Combined) | PYLGDRRSYGFDH |
| SEQ ID NO: 409 | HCDR1 (Kabat) | TYAMT |
| SEQ ID NO: 408 | HCDR2 (Kabat) | SISASGYYANYAGSVKG |
| SEQ ID NO: 331 | HCDR3 (Kabat) | PYLGDRRSYGFDH |
| SEQ ID NO: 410 | HCDR1 (Chothia) | GFTFGTY |
| SEQ ID NO: 411 | HCDR2 (Chothia) | SASGYY |
| SEQ ID NO: 331 | HCDR3 (Chothia) | PYLGDRRSYGFDH |
| SEQ ID NO: 412 | HCDR1 (IMGT) | GFTFGTYA |
| SEQ ID NO: 413 | HCDR2 (IMGT) | ISASG |
| SEQ ID NO: 332 | HCDR3 (IMGT) | ARPYLGDRRSYGFDH |
| SEQ ID NO: 414 | VH | EVQLLESGGGLVQPGGSLRLSCAASGFTFGTYAMTWVRQAPGKGLEWVSS |
| | | ISASGYYANYAGSVKGRFTISRDNSKNTLYLQMNSLRAEDTAVYYCARPY |
| | | LGDRRSYGFDHWGQGTLVTVSS |
| SEQ ID NO: 415 | DNA VH | gaagtgcagctgctggaaagcggtggcggtctggtgcagccaggtggtagcctgcgcctgagctgtgccgcaagcg |
| | | ggtttacattcggcacctatgcaatgacttgggtgcgccaagcaccaggcaaaggcctggaatgggtgagtagcattag |
| | | cgcatccggatattacgctaactacgcaggcagcgtcaaaggccgctttaccattagtcgcgataacagcaaaaacacc |
| | | ctgtatctgcaaatgaacagcctgcgggcagaagataccgcagtttattattgcgcgcgaccttatctgggtgaccgtcg |
| | | tagctatggtttcgaccactggggccagggcaccctggttactgtctcgagc |
| SEQ ID NO: 416 | Heavy Chain | EVQLLESGGGLVQPGGSLRLSCAASGFTFGTYAMTWVRQAPGKGLEWVSS |
| | | ISASGYYANYAGSVKGRFTISRDNSKNTLYLQMNSLRAEDTAVYYCARPY |
| | | LGDRRSYGFDHWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTY |
| | | ICNVNHKPSNTKVDKRVEPKSCDKTHTCPPCPAPEAAGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 417 | DNA Heavy Chain | gaagtgcagctgctggaaagcggtggcggtctggtgcagccaggtggtagcctgcgcctgagctgtgccgcaagcg |
| | | ggtttacattcggcacctatgcaatgacttgggtgcgccaagcaccaggcaaaggcctggaatgggtgagtagcattag |
| | | cgcatccggatattacgctaactacgcaggcagcgtcaaaggccgctttaccattagtcgcgataacagcaaaaacacc |
| | | ctgtatctgcaaatgaacagcctgcgggcagaagataccgcagtttattattgcgcgcgaccttatctgggtgaccgtcg |
| | | tagctatggtttcgaccactggggccagggcaccctggttactgtctcgagcgcgtcgaccaaaggccccagcgtgttc |
| | | cctctggcccccagcagcaagagcacctctggcggaacagccgccctgggctgcctggtcaaggactacttccccga |
| | | gcccgtgaccgtgtcctggaactctggcgccctgaccagcggcgtgcacacctttccagccgtgctccagagcagcg |
| | | gcctgtacagcctgagcagcgtcgtgaccgtgcccagcagcagcctgggcacccagacctacatctgcaacgtgaac |
| | | cacaagcccagcaacacaaaggtggacaaggggtggaacccaagagctgcgacaagacccacacctgtcccccc |
| | | tgccctgcccctgaagcgggggaggcccctccgtgttcctgttccccccaaagcctaaggacaccctgatgatcagc |
| | | cggacccccgaagtgacctgcgtggtggtggacgtgtcccacgaggaccctgaagtgaagtttaattggtacgtggac |
| | | ggcgtggaagtgcacaacgccaagaccaagcccagagaggaacagtacaacagcacctaccgggtggtgtccgtg |
| | | ctgaccgtgctgcaccaggactggctgaacggcaaagagtacaagtgcaaggtgtccaacaaggccctgcctgcccc |
| | | catcgagaaaaccatcagcaaggccaaaggccagccccgcgagccccaggtgtacacactgccccctagccggga |
| | | agagatgaccaagaaccaggtgtccctgacctgcctcgtgaagggcttctaccccagcgacattgccgtggaatggga |
| | | gagcaacggccagcccgagaacaactacaagaccaccccccctgtgctggacagcgacggctcattcttcctgtaca |
| | | gcaagctgaccgtggacaagagccggtggcagcagggcaacgtgttcagctgctccgtgatgcacgaggccctgca |
| | | caaccactacacccagaagtccctgagcctgagccccggcaag |
| SEQ ID NO: 337 | LCDR1 (Combined) | TGTSSDVGSYNLVS |
| SEQ ID NO: 338 | LCDR2 (Combined) | EGSKRPS |
| SEQ ID NO: 339 | LCDR3 (Combined) | SSYGFHIVVVV |
| SEQ ID NO: 337 | LCDR1 (Kabat) | TGTSSDVGSYNLVS |
| SEQ ID NO: 338 | LCDR2 (Kabat) | EGSKRPS |
| SEQ ID NO: 339 | LCDR3 (Kabat) | SSYGFHIVVVV |
| SEQ ID NO: 340 | LCDR1 (Chothia) | TSSDVGSYNL |
| SEQ ID NO: 341 | LCDR2 (Chothia) | EGS |
| SEQ ID NO: 342 | LCDR3 (Chothia) | YGFHIVVV |
| SEQ ID NO: 343 | LCDR1 (IMGT) | SSDVGSYNL |
| SEQ ID NO: 341 | LCDR2 (IMGT) | EGS |
| SEQ ID NO: 339 | LCDR3 (IMGT) | SSYGFHIVVVV |
| SEQ ID NO: 344 | VL | QSALTQPASVSGSPGQSITISCTGTSSDVGSYNLVSWYQQHPGKAPKLMIYE |
| | | GSKRPSGVSNRFSGSKSGNTASLTISGLQAEDEADYYCSSYGFHIVVVVFGG |
| | | GTKLTVL |
| SEQ ID NO: 345 | DNA VL | cagagcgccctgacccagccagccagcgttagcggtagcccaggccagagcattaccattagctgcaccggcacca |
| | | gcagcgacgtgggcagctataacctggttagctggtatcagcagcatccgggcaaagccccgaaactgatgatctatg |
| | | aaggcagcaaacgcccgagcggcgttagcaaccgctttagtggcagcaaaagcggcaacaccgccagcctgaccat |
| | | tagcggcctgcaagccgaagacgaagccgattattactgctcctcttacggtttccatatcgttgttgttgtgtttggcggc |
| | | ggtaccaagctgaccgtgctg |
| SEQ ID NO: 346 | Light Chain | QSALTQPASVSGSPGQSITISCTGTSSDVGSYNLVSWYQQHPGKAPKLMIYE |
| | | GSKRPSGVSNRFSGSKSGNTASLTISGLQAEDEADYYCSSYGFHIVVVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| SEQ ID NO: 347 | DNA Light Chain | cagagcgccctgacccagccagccagcgttagcggtagcccaggccagagcattaccattagctgcaccggcacca |
| | | gcagcgacgtgggcagctataacctggttagctggtatcagcagcatccgggcaaagccccgaaactgatgatctatg |
| | | aaggcagcaaacgcccgagcggcgttagcaaccgctttagtggcagcaaaagcggcaacaccgccagcctgaccat |
| | | tagcggcctgcaagccgaagacgaagccgattattactgctcctcttacggtttccatatcgttgttgttgtgtttggcggc |
| | | ggtaccaagctgaccgtgctgggccagcccaaagccgcccctagcgtgaccctgttccccccaagcagcgaggaac |
| | | tccaggccaacaaggccaccctcgtgtgcctgatcagcgacttctaccctggcgccgtgaccgtggcctggaaggcc |
| | | gatagcagccctgtgaaggccggcgtggaaaccaccacccccagcaagcagagcaacaacaaatacgccgccagc |
| | | agctacctgagcctgacccccgagcagtggaagtcccacagatcctacagctgccaggtcacacacgagggcagca |
| | | ccgtggaaaagaccgtggcccccaccgagtgcagc |
| ZZ17_LALA | | |
| SEQ ID NO: 418 | HCDR1 (Combined) | GFTFSDYAIS |
| SEQ ID NO: 419 | HCDR2 (Combined) | SISGGGYHTQYAGSVKG |
| SEQ ID NO: 331 | HCDR3 (Combined) | PYLGDRRSYGFDH |
| SEQ ID NO: 420 | HCDR1 (Kabat) | DYAIS |
| SEQ ID NO: 419 | HCDR2 (Kabat) | SISGGGYHTQYAGSVKG |
| SEQ ID NO: 331 | HCDR3 (Kabat) | PYLGDRRSYGFDH |
| SEQ ID NO: 421 | HCDR1 (Chothia) | GFTFSDY |
| SEQ ID NO: 422 | HCDR2 (Chothia) | SGGGYH |
| SEQ ID NO: 331 | HCDR3 (Chothia) | PYLGDRRSYGFDH |
| SEQ ID NO: 423 | HCDR1 (IMGT) | GFTFSDYA |
| SEQ ID NO: 424 | HCDR2 (IMGT) | ISGGGYHT |
| SEQ ID NO: 332 | HCDR3 (IMGT) | ARPYLGDRRSYGFDH |
| SEQ ID NO: 425 | VH | EVQLLESGGGLVQPGGSLRLSCAASGFTFSDYAISWVRQAPGKGLEWVSSI |
| | | SGGGYHTQYAGSVKGRFTISRDNSKNTLYLQMNSLRAEDTAVYYCARPYL |
| | | GDRRSYGFDHWGQGTLVTVSS |
| SEQ ID NO: 426 | DNA VH | gaagtgcagctgctggaaagcggtggcggtctggtgcagccaggtggtagcctgcgcctgagctgtgccgcaagcg |
| | | gctttaccttttccgactatgcaatcagctgggtgcgccaagcaccaggcaaaggcctggaatgggtgagcagcatttc |
| | | cgggggggggtatcatacacaatatgcaggatccgtgaaaggccgctttaccattagtcgcgataacagcaaaaacac |
| | | cctgtatctgcaaatgaacagcctgcgggcagaagataccgcagtttattattgcgcgcgaccttatctgggtgaccgtc |
| | | gtagctatggtttcgaccactggggccagggcaccctggttactgtctcgagc |
| SEQ ID NO: 427 | Heavy Chain | EVQLLESGGGLVQPGGSLRLSCAASGFTFSDYAISWVRQAPGKGLEWVSSI |
| | | SGGGYHTQYAGSVKGRFTISRDNSKNTLYLQMNSLRAEDTAVYYCARPYL |
| | | GDRRSYGFDHWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYI |
| | | CNVNHKPSNTKVDKRVEPKSCDKTHTCPPCPAPEAAGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 428 | DNA Heavy Chain | gaagtgcagctgctggaaagcggtggcggtctggtgcagccaggtggtagcctgcgcctgagctgtgccgcaagcg |
| | | gctttaccttttccgactatgcaatcagctgggtgcgccaagcaccaggcaaaggcctggaatgggtgagcagcatttc |
| | | ggggggtatcatacacaatatgcaggatccgtgaaaggccgctttaccattagtcgcgataacagcaaaaacac |
| | | cctgtatctgcaaatgaacagcctgcgggcagaagataccgcagtttattattgcgcgcgaccttatctgggtgaccgtc |
| | | gtagctatggtttcgaccactggggccagggcaccctggttactgtctcgagcgcgtcgaccaaaggccccagcgtgtt |
| | | ccctctggcccccagcagcaagagcacctctggcggaacagccgccctgggctgcctggtcaaggactacttccccg |
| | | agcccgtgaccgtgtcctggaactctggcgccctgaccagcggcgtgcacacctttccagccgtgctccagagcagc |
| | | ggcctgtacagcctgagcagcgtcgtgaccgtgcccagcagcagcctgggcacccagacctacatctgcaacgtgaa |
| | | ccacaagcccagcaacacaaaggtggacaagcgggtggaacccaagagctgcgacaagacccacacctgtccccc |
| | | ctgccctgcccctgaagcgggggaggcccctccgtgttcctgttccccccaaagcctaaggacaccctgatgatcag |
| | | ccggacccccgaagtgacctgcgtggtggtggacgtgtcccacgaggaccctgaagtgaagtttaattggtacgtgga |
| | | cggcgtggaagtgcacaacgccaagaccaagcccagagaggaacagtacaacagcacctaccgggtggtgtccgt |
| | | gctgaccgtgctgcaccaggactggctgaacggcaaagagtacaagtgcaaggtgtccaacaaggccctgcctgccc |
| | | ccatcgagaaaaccatcagcaaggccaaaggccagccccgcgagccccaggtgtacacactgccccctagccggg |
| | | aagagatgaccaagaaccaggtgtccctgacctgcctcgtgaagggcttctaccccagcgacattgccgtggaatggg |
| | | agagcaacggccagcccgagaacaactacaagaccaccccccctgtgctggacagcgacggctcattcttcctgtac |
| | | agcaagctgaccgtggacaagagccggtggcagcagggcaacgtgttcagctgctccgtgatgcacgaggccctgc |
| | | acaaccactacacccagaagtccctgagcctgagccccggcaag |
| SEQ ID NO: 337 | LCDR1 (Combined) | TGTSSDVGSYNLVS |
| SEQ ID NO: 338 | LCDR2 (Combined) | EGSKRPS |
| SEQ ID NO: 339 | LCDR3 (Combined) | SSYGFHIVVVV |
| SEQ ID NO: 337 | LCDR1 (Kabat) | TGTSSDVGSYNLVS |
| SEQ ID NO: 338 | LCDR2 (Kabat) | EGSKRPS |
| SEQ ID NO: 339 | LCDR3 (Kabat) | SSYGFHIVVVV |
| SEQ ID NO: 340 | LCDR1 (Chothia) | TSSDVGSYNL |
| SEQ ID NO: 341 | LCDR2 (Chothia) | EGS |
| SEQ ID NO: 342 | LCDR3 (Chothia) | YGFHIVVV |
| SEQ ID NO: 343 | LCDR1 (IMGT) | SSDVGSYNL |
| SEQ ID NO: 341 | LCDR2 (IMGT) | EGS |
| SEQ ID NO: 339 | LCDR3 (IMGT) | SSYGFHIVVVV |
| SEQ ID NO: 344 | VL | QSALTQPASVSGSPGQSITISCTGTSSDVGSYNLVSWYQQHPGKAPKLMIYE |
| | | GSKRPSGVSNRFSGSKSGNTASLTISGLQAEDEADYYCSSYGFHIVVVVFGG |
| | | GTKLTVL |
| SEQ ID NO: 345 | DNA VL | cagagcgccctgacccagccagccagcgttagcggtagcccaggccagagcattaccattagctgcaccggcacca |
| | | gcagcgacgtgggcagctataacctggttagctggtatcagcagcatccgggcaaagccccgaaactgatgatctatg |
| | | aaggcagcaaacgcccgagcggcgttagcaaccgctttagtggcagcaaaagcggcaacaccgccagcctgaccat |
| | | tagcggcctgcaagccgaagacgaagccgattattactgctcctcttacggtttccatatcgttgttgttgtgtttggcggc |
| | | ggtaccaagctgaccgtgctg |
| SEQ ID NO: 346 | Light Chain | QSALTQPASVSGSPGQSITISCTGTSSDVGSYNLVSWYQQHPGKAPKLMIYE |
| | | GSKRPSGVSNRFSGSKSGNTASLTISGLQAEDEADYYCSSYGFHIVVVVFGG |
| | | GTKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGST |
| | | VEKTVAPTECS |
| SEQ ID NO: 347 | DNA Light Chain | cagagcgccctgacccagccagccagcgttagcggtagcccaggccagagcattaccattagctgcaccggcacca |
| | | gcagcgacgtgggcagctataacctggttagctggtatcagcagcatccgggcaaagccccgaaactgatgatctatg |
| | | aaggcagcaaacgcccgagcggcgttagcaaccgctttagtggcagcaaaagcggcaacaccgccagcctgaccat |
| | | tagcggcctgcaagccgaagacgaagccgattattactgctcctcttacggtttccatatcgttgttgttgtgtttggcggc |
| | | ggtaccaagctgaccgtgctgggccagcccaaagccgcccctagcgtgaccctgttccccccaagcagcgaggaac |
| | | tccaggccaacaaggccaccctcgtgtgcctgatcagcgacttctaccctggcgccgtgaccgtggcctggaaggcc |
| | | gatagcagccctgtgaaggccggcgtggaaaccaccacccccagcaagcagagcaacaacaaatacgccgccagc |
| | | agctacctgagcctgacccccgagcagtggaagtcccacagatcctacagctgccaggtcacacacgagggcagca |
| | | ccgtggaaaagaccgtggcccccaccgagtgcagc |

As a non-limiting set of examples, an antibody named herein may be described in any suitable manner with reference to the sequences described in Table 2. For example, each of the antibodies may be defined as having three heavy chain complementarity determining regions (HCDR1, HCDR2, and HCDR3) and three light chain complementarity determining regions (LCDR1, LCDR2, and LCDR3) for any of the numbering schemes described in Table 2. Each of the antibodies may be described as comprising or having one of the combinations of a heavy chain variable region and a light chain variable region described in Table 2. Each of the antibodies may be described as comprising or having one of the combinations of a heavy chain and a light chain described in Table 2. Each of the antibodies may be described as comprising or having a set of CDRs present in any one of the sets of a heavy chain and a light chain described in Table 2.

For example, XX16_LALA may be defined as having three heavy chain complementarity determining regions (HCDR1, HCDR2, and HCDR3) and three light chain complementarity determining regions (LCDR1, LCDR2, and LCDR3) selected from: (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3), (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 135 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 134 (LCDR3). In some embodiments, XX16_LALA may be defined as comprising or having amino acid sequences of SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3). In some embodiments, XX16_LALA may be defined as comprising or having amino acid sequences of SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3). In some embodiments, XX16_LALA may be defined as comprising or having amino acid sequences of SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 135 (LCDR3). In some embodiments, XX16_LALA may be defined as comprising or having amino acid sequences of SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 134 (LCDR3). In some embodiments, XX16_LALA may be defined as comprising or having a heavy chain variable region comprising or having an amino acid sequence of SEQ ID NO: 201, and a light chain variable region comprising or having an amino acid sequence of SEQ ID NO: 136. In some embodiments, XX16_LALA may be defined as comprising or having a heavy chain comprising or having an amino acid sequence of SEQ ID NO: 203, and a light chain comprising or having an amino acid sequence of SEQ ID NO: 138.

For example, XX16_DAPA may be defined as having three heavy chain complementarity determining regions (HCDR1, HCDR2, and HCDR3) and three light chain complementarity determining regions (LCDR1, LCDR2, and LCDR3) selected from: (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 135 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 134 (LCDR3). In some embodiments, XX16_DAPA may be defined as comprising or having amino acid sequences of SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3). In some embodiments, XX16_DAPA may be defined as comprising or having amino acid sequences of SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3). In some embodiments, XX16_DAPA may be defined as comprising or having amino acid sequences of SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 135 (LCDR3). In some embodiments, XX16_DAPA may be defined as comprising or having amino acid sequences of SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 134 (LCDR3). In some embodiments, XX16_DAPA may be defined as comprising or having a heavy chain variable region comprising or having an amino acid sequence of SEQ ID NO: 122, and a light chain variable region comprising or having an amino acid sequence of SEQ ID NO: 136. In some embodiments, XX16_DAPA may be defined as comprising or having a heavy chain comprising or having an amino acid sequence of SEQ ID NO: 208, and a light chain comprising or having an amino acid sequence of SEQ ID NO: 138.

In some embodiments, the anti-NPR1 antibody or antigen binding fragment may be defined as having or comprising a heavy chain variable region comprising or having an amino acid sequence of one of the sequences of Table 2 or a sequence having at least 95%, 96%, 97%, 98%, or 99% identity with that sequence, and a light chain variable region comprising or having an amino acid sequence of the light chain variable region of that anti-NPR1 antibody or antigen binding fragment or a sequence having at least 95%, 96%, 97%, 98%, or 99% identity with the light chain variable region sequence. In some other embodiments, the anti-NPR1 antibody or antigen binding fragment may be defined as having or comprising a heavy chain comprising or having an amino acid sequence of one of the heavy chain sequences in Table 2 or a sequence having at least 95%, 96%, 97%, 98%, or 99% identity with that heavy chain sequence, and a light chain comprising or having an amino acid sequence of the light chain of that anti-NPR1 antibody or antigen binding fragment or a sequence having at least 95%, 96%, 97%, 98%, or 99% identity with the light chain.

In some embodiments, the anti-NPR1 antibody or antigen binding fragment comprises a heavy chain variable region comprising or having an amino acid sequence of SEQ ID NO: 13 or a sequence having at least 95%, 96%, 97%, 98%, or 99% identity with SEQ ID NO: 13, and a light chain variable region comprising or having an amino acid sequence of SEQ ID NO: 24 or a sequence having at least 95%, 96%, 97%, 98%, or 99% identity with SEQ ID NO: 24. In some other embodiments, the anti-NPR1 antibody or antigen binding fragment comprises a heavy chain comprising or having an amino acid sequence of SEQ ID NO: 15 or a sequence having at least 95%, 96%, 97%, 98%, or 99% identity with SEQ ID NO: 15, and a light chain comprising or having an amino acid sequence of SEQ ID NO: 26 or a sequence having at least 95%, 96%, 97%, 98%, or 99% identity with SEQ ID NO: 26.

In some embodiments, the anti-NPR1 antibody or antigen binding fragment comprises a heavy chain variable region comprising or having an amino acid sequence of SEQ ID NO: 122 or a sequence having at least 95%, 96%, 97%, 98%, or 99% identity with SEQ ID NO: 122, and a light chain variable region comprising or having an amino acid sequence of SEQ ID NO: 136 or a sequence having at least 95%, 96%, 97%, 98%, or 99% identity with SEQ ID NO: 136. In some embodiments, the anti-NPR1 antibody or antigen binding fragment comprises a heavy chain comprising or having an amino acid sequence of SEQ ID NO: 208 or a sequence having at least 95%, 96%, 97%, 98%, or 99% identity with SEQ ID NO: 208, and a light chain comprising or having an amino acid sequence of SEQ ID NO: 138 or a sequence having at least 95%, 96%, 97%, 98%, or 99% identity with SEQ ID NO: 138.

In some embodiments, an anti-NPR1 (or target) antibody or antigen-binding fragment thereof as provided herein binds to (a) human NPR1; and (b) mouse NPR1 and/or rat NPR1. In some embodiments, an anti-NPR1 (or target) antibody or antigen-binding fragment thereof as provided herein binds to (a) human NPR1; and (b) cyno NPR1. In some embodiments, the anti-NPR1 (or target) antibody or antigen binding fragment thereof is therapeutic. A therapeutic antibody, as defined herein, is an antibody that is both efficacious and stable.

Binding Agents that Bind to the Anti-NPR1 Antibodies and Antigen Binding Fragments Thereof Described Herein

As a non-limiting set of examples, a binding agent or a reversal agent named herein may be described in any suitable manner with reference to an anti-NPR1 antibody or antigen binding fragment thereof described herein (e.g., with reference to the sequences described in Table 2). For example, each of the binding agents or anti-idiotype antibodies described herein may be defined as binding to an anti-NPR1 antibody or antigen binding fragment thereof described herein. Such an anti-NPR1 antibody may be defined, for example, as having three heavy chain complementarity determining regions (HCDR1, HCDR2, and HCDR3) and three light chain complementarity determining regions (LCDR1, LCDR2, and LCDR3) for any of the numbering schemes described in Table 2. Such an anti-NPR1 antibody may be defined, for example, as comprising or having one of the combinations of a heavy chain variable region and a light chain variable region described in Table 2. Such an anti-NPR1 antibody may be defined, for example, as comprising or having one of the combinations of a heavy chain and a light chain described in Table 2. Such an anti-NPR1 antibody may be defined, for example, as comprising or having a set of CDRs present in any one of the sets of a heavy chain and a light chain described in Table 2.

For example, the binding agent may specifically bind to XX16_LALA, which may be defined as having three heavy chain complementarity determining regions (HCDR1, HCDR2, and HCDR3) and three light chain complementarity determining regions (LCDR1, LCDR2, and LCDR3) selected from: (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 135 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 134 (LCDR3). In some embodiments, the binding agent may specifically bind to XX16_LALA, which may be defined as comprising or having amino acid sequences of SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3). In some embodiments, the binding agent may specifically bind to XX16_LALA, which may be defined as comprising or having amino acid sequences of SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3). In some embodiments, the binding agent may specifically bind to XX16_LALA, which may be defined as comprising or having amino acid sequences of SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 135 (LCDR3). In some embodiments, the binding agent may specifically bind to XX16_LALA, which may be defined as comprising or having amino acid sequences of SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 134 (LCDR3). In some embodiments, the binding agent may specifically bind to XX16_LALA, which may be defined as comprising or having a heavy chain variable region comprising or having an amino acid sequence of SEQ ID NO: 201, and a light chain variable region comprising or having an amino acid sequence of SEQ ID NO: 136. In some embodiments, the binding agent may specifically bind to XX16_LALA, which may be defined as comprising or having a heavy chain comprising or having an amino acid sequence of SEQ ID NO: 203, and a light chain comprising or having an amino acid sequence of SEQ ID NO: 138.

For example, the binding agent may specifically bind to XX16_DAPA, which may be defined as having three heavy chain complementarity determining regions (HCDR1, HCDR2, and HCDR3) and three light chain complementarity determining regions (LCDR1, LCDR2, and LCDR3) selected from: (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 135 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 134 (LCDR3). In some embodiments, the binding agent may specifically bind to XX16_DAPA, which may be defined as comprising or having amino acid sequences of SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3). In some embodiments, the binding agent may specifically bind to XX16_DAPA, which may be defined as comprising or having amino acid sequences of SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3). In some embodiments, the binding agent may specifically bind to XX16_DAPA, which may be defined as comprising or having amino acid sequences of SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 135 (LCDR3). In some embodiments, the binding agent may specifically bind to XX16_DAPA, which may be defined as comprising or having amino acid sequences of SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 134 (LCDR3). In some embodiments, the binding agent may specifically bind to XX16_DAPA, which may be defined as comprising or having a heavy chain variable region comprising or having an amino acid sequence of SEQ ID NO: 122, and a light chain variable region comprising or having an amino acid sequence of SEQ ID NO: 136. In some embodiments, the binding agent may specifically bind to XX16_DAPA, which may be defined as comprising or having a heavy chain comprising or having an amino acid sequence of SEQ ID NO: 208, and a light chain comprising or having an amino acid sequence of SEQ ID NO: 138.

In some embodiments, the binding agent may specifically bind to an anti-NPR1 antibody or antigen binding fragment which may be defined as having or comprising a heavy chain variable region comprising or having an amino acid sequence of one of the sequences of Table 2 or a sequence having at least 95%, 96%, 97%, 98%, or 99% identity with that sequence, and a light chain variable region comprising or having an amino acid sequence of the light chain variable region of that anti-NPR1 antibody or antigen binding fragment or a sequence having at least 95%, 96%, 97%, 98%, or 99% identity with the light chain variable region sequence. In some other embodiments, the binding agent may specifically bind to an anti-NPR1 antibody or antigen binding fragment, which may be defined as having or comprising a heavy chain comprising or having an amino acid sequence of one of the heavy chain sequences in Table 2 or a sequence having at least 95%, 96%, 97%, 98%, or 99% identity with that heavy chain sequence, and a light chain comprising or having an amino acid sequence of the light chain of that anti-NPR1 antibody or antigen binding fragment or a sequence having at least 95%, 96%, 97%, 98%, or 99% identity with the light chain.

In some embodiments, the binding agent may specifically bind to an anti-NPR1 antibody or antigen binding fragment which comprises a heavy chain variable region comprising or having an amino acid sequence of SEQ ID NO: 13 or a sequence having at least 95%, 96%, 97%, 98%, or 99% identity with SEQ ID NO: 13, and a light chain variable region comprising or having an amino acid sequence of SEQ ID NO: 24 or a sequence having at least 95%, 96%, 97%, 98%, or 99% identity with SEQ ID NO: 24. In some other embodiments, the binding agent may specifically bind to an anti-NPR1 antibody or antigen binding fragment which comprises a heavy chain comprising or having an amino acid sequence of SEQ ID NO: 15 or a sequence having at least 95%, 96%, 97%, 98%, or 99% identity with SEQ ID NO: 15, and a light chain comprising or having an amino acid sequence of SEQ ID NO: 26 or a sequence having at least 95%, 96%, 97%, 98%, or 99% identity with SEQ ID NO: 26.

In some embodiments, the binding agent may specifically bind to an anti-NPR1 antibody or antigen binding fragment which comprises a heavy chain variable region comprising or having an amino acid sequence of SEQ ID NO: 122 or a sequence having at least 95%, 96%, 97%, 98%, or 99% identity with SEQ ID NO: 122, and a light chain variable region comprising or having an amino acid sequence of SEQ ID NO: 136 or a sequence having at least 95%, 96%, 97%, 98%, or 99% identity with SEQ ID NO: 136. In some embodiments, the binding agent may specifically bind to an anti-NPR1 antibody or antigen binding fragment which comprises a heavy chain comprising or having an amino acid sequence of SEQ ID NO: 208 or a sequence having at least 95%, 96%, 97%, 98%, or 99% identity with SEQ ID NO: 208, and a light chain comprising or having an amino acid sequence of SEQ ID NO: 138 or a sequence having at least 95%, 96%, 97%, 98%, or 99% identity with SEQ ID NO: 138.

In some embodiments, the binding agent may specifically bind to an anti-NPR1 (or target) antibody or antigen-binding fragment thereof as provided herein which binds to (a) human NPR1; and (b) mouse NPR1 and/or rat NPR1. In some embodiments, the binding agent may specifically bind to an anti-NPR1 (or target) antibody or antigen-binding fragment thereof as provided herein which binds to (a) human NPR1; and (b) cyno NPR1. In some embodiments, the binding agent may specifically bind to an anti-NPR1 (or target) antibody or antigen binding fragment thereof which is therapeutic. A therapeutic antibody, as defined herein, is an antibody that is both efficacious and stable.

Antibodies that Bind to the Same Epitope as the Described Anti-NPR1 Antibodies; and Binding Agents Targeting the Same

In another embodiment, provided herein are anti-NPR1 (or target) antibodies or antigen-binding fragments thereof that bind to the same epitope as one or more of the anti-NPR1 antibodies described herein (e.g., WW06). Such antibodies:
(i) bind NPR1;
(ii) are agonists of NPR1;
(iii) are ANP competitive; and
bind the same epitope in NPR1 as antibody WW06.

In another embodiment, provided herein are anti-NPR1 (or target) antibodies or antigen-binding fragments thereof that bind to the same epitope as one or more of the anti-NPR1 antibodies described herein (e.g., XX16). Such antibodies:
(i) bind NPR1;
(ii) are agonists of NPR1;
(iii) are ANP non-competitive; and
bind the same epitope in NPR1 as XX16.

In another embodiment, provided herein are anti-NPR1 (or target) antibodies or antigen-binding fragments thereof that bind to the same epitope as one or more of the anti-NPR1 antibodies described herein (e.g., WW03). Such antibodies:
(i) bind NPR1;
(ii) are agonists of NPR1;
(iii) are ANP non-competitive; and
bind the same epitope in NPR1 as WW03.

In another embodiment, provided herein are binding agents that specifically bind anti-NPR1 (or target) antibodies or antigen-binding fragments thereof which bind to the same epitope as one or more of the anti-NPR1 antibodies described herein (e.g., WW06). Such antibodies:
(i) bind NPR1;
(ii) are agonists of NPR1;
(iii) are ANP competitive; and
bind the same epitope in NPR1 as antibody WW06.

In another embodiment, provided herein are binding agents that specifically bind anti-NPR1 (or target) antibodies or antigen-binding fragments thereof which bind to the same epitope as one or more of the anti-NPR1 antibodies described herein (e.g., XX16). Such antibodies:
(i) bind NPR1;
(ii) are agonists of NPR1;
(iii) are ANP non-competitive; and
bind the same epitope in NPR1 as XX16.

In another embodiment, provided herein are provided herein are binding agents that specifically bind anti-NPR1 (or target) antibodies or antigen-binding fragments thereof which bind to the same epitope as one or more of the anti-NPR1 antibodies described herein (e.g., WW03). Such antibodies:
(i) bind NPR1;
(ii) are agonists of NPR1;
(iii) are ANP non-competitive; and
bind the same epitope in NPR1 as WW03.

Following the crystallisation and structure determination, the binding regions of the preferred antibodies of the disclosure have been more clearly defined. Such binding is defined herein as being inclusive of both covalent and non-covalent bonds.

Also provided herein are binding agents that bind to an anti-NPR1 (or target) ANP competitive antibody that binds the same epitope as WW06. In some embodiments, provided herein are binding agents that bind to an anti-NPR1 (or target) antibody that binds to an epitope of human NPR1 protein (Accession no. P16066; SEQ ID NO: 1) comprising amino acids 188, 192, 194, 197, 201, 208, and 219. In some embodiments, provided herein are binding agents that bind to an anti-NPR1 (or target) antibody that binds to an epitope of human NPR1 protein (Accession no. P16066; SEQ ID NO: 1) comprising amino acids 188, 192, 194, 197, 201, 208, 219, and 295. In some embodiments, provided herein are binding agents that bind to an anti-NPR1 (or target) antibody that binds to an epitope within amino acid numbers 188-198 of SEQ ID NO: 1. In some embodiments, provided herein are binding agents that bind to an anti-NPR1 (or target) antibody that binds to at least 2, 3, or 4 amino acid residues within amino acid numbers 188-198 of SEQ ID NO: 1. In some embodiments, provided herein are binding agents that bind to an anti-NPR1 (or target) antibody that binds to an epitope within amino acid numbers 201-208 of SEQ ID NO: 1. In some embodiments, provided herein are binding agents that bind to an anti-NPR1 (or target) antibody that binds to at least 2 amino acids within amino acid numbers 201-208 of SEQ ID NO: 1. In some embodiments, provided herein are binding agents that bind to an anti-NPR1 (or target) antibody that binds to at least 2, 3, or 4 amino acid residues within amino acid numbers 188-198 of SEQ ID NO: 1, and binds to at least 2 amino acids within amino acid numbers 201-208 of SEQ ID NO: 1. In some embodiments, provided herein are binding agents that bind to an anti-NPR1 (or target) antibody that binds to an epitope comprising at least one amino acid residue within each of (i) amino acids 188-198 of SEQ ID NO: 1, (ii) amino acids 201-208 of SEQ ID NO: 1, (iii) amino acids 215-238 of SEQ ID NO: 1, and (iv) amino acids 294-297 of SEQ ID NO: 1.

In some embodiments, provided herein are binding agents that bind to an anti-NPR1 ANP non-competitive antibody that binds the same epitope as WW03. In some embodiments, provided herein are binding agents that bind to an anti-NPR1 antibody that binds to an epitope of human NPR1 protein (Accession no. P16066; SEQ ID NO: 1) comprising amino acids 82, 102, 103, 105, 106, 109, 132, and 375. In some embodiments, provided herein are binding agents that bind to an anti-NPR1 antibody that binds to an epitope of human NPR1 protein (Accession no. P16066; SEQ ID NO: 1) comprising amino acids 79, 82, 99, 102, 103, 105, 106, 109, 131, 132, and 375. In some embodiments, provided herein are binding agents that bind to an anti-NPR1 antibody that binds to an epitope within amino acid numbers 99-111 of SEQ ID NO: 1. In some embodiments, provided herein are binding agents that bind to an anti-NPR1 that binds to at least 2, 3, 4, 5, or 6 amino acids within amino acid numbers 99-111 of SEQ ID NO: 1. In some embodiments, provided herein are binding agents that bind to an anti-NPR1 antibody that binds to at least 2, 3, 4, 5, 6, 7, or 8 amino acid residues within amino acid numbers 99-133 of SEQ ID NO: 1. In some embodiments, provided herein are binding agents that bind to an anti-NPR1 antibody that binds to an epitope within amino acid numbers 131-134 of SEQ ID NO: 1. In some embodiments, provided herein are binding agents that bind to an anti-NPR1 antibody that binds to at least 2 amino acids within amino acid numbers 131-134 of SEQ ID NO: 1. In some embodiments, provided herein are binding agents that bind to an anti-NPR1 antibody that binds to at least 2, 3, 4, 5, or 6 amino acids within amino acid numbers 99-111 of SEQ ID NO: 1, and binds to at least 2 amino acids within amino acid numbers 131-134 of SEQ ID NO: 1. In some embodiments, provided herein are binding agents that bind to an anti-NPR1 antibody that binds to an epitope comprising at least one amino acid residue within each of (i) amino acids 99-111 of SEQ ID NO: 1, (ii) amino acids 131-134 of SEQ ID NO: 1, and (iii) amino acids 374-375 of SEQ ID NO: 1. Optionally, the anti-NPR1 antibody may additionally bind to amino acids 79 and/or 82 of SEQ ID NO: 1.

In some embodiments, provided herein are binding agents that bind to an anti-NPR1 ANP non-competitive antibody that binds the same epitope as XX16. In some embodiments, provided herein are binding agents that bind to an anti-NPR1 antibody that binds to an epitope of human NPR1 protein (Accession no. P16066; SEQ ID NO: 1) comprising amino acids 82, 102, 103, 105, 106, 109, 132, and 375. In some embodiments, provided herein are binding agents that bind to an anti-NPR1 antibody that binds to an epitope of human NPR1 protein (Accession no. P16066; SEQ ID NO: 1) comprising amino acids 34, 82, 102, 103, 105, 106, 107, 109, 132, 133, 375, and 378. In some embodiments, provided herein are binding agents that bind to an anti-NPR1 antibody that binds to an epitope within amino acid numbers 102-111 of SEQ ID NO: 1. In some embodiments, provided herein are binding agents that bind to an anti-NPR1 antibody that binds to at least 2, 3, 4, 5, or 6 amino acids within amino acid numbers 102-111 of SEQ ID NO: 1. In some embodiments, provided herein are binding agents that bind to an anti-NPR1 antibody that binds to an epitope within amino acid numbers 131-134 of SEQ ID NO: 1. In some embodiments, provided herein are binding agents that bind to an anti-NPR1 antibody that binds to at least 2 amino acids within amino acid numbers 131-134 of SEQ ID NO: 1. In some embodiments, provided herein are binding agents that bind to an anti-NPR1 antibody that binds to at least 2, 3, 4, 5, or 6 amino acids within amino acid numbers 102-111 of SEQ ID NO: 1, and binds to at least 2 amino acids within amino acid numbers 131-134 of SEQ ID NO: 1. In some embodiments, provided herein are binding agents that bind to an anti-NPR1 antibody that binds to an epitope comprising at least one amino acid residue within each of (i) amino acids 102-111 of SEQ ID NO: 1, (ii) amino acids 131-134 of SEQ ID NO: 1, and (iii) amino acids 374-378 of SEQ ID NO: 1. Optionally, the anti-NPR1 antibody may additionally bind to amino acids 34, 76, and/or 82 of SEQ ID NO: 1.

Additional anti-NPR or target antibodies can be identified based on their ability to cross-compete (e.g., to competitively inhibit the binding of, in a statistically significant manner) with other antibodies described herein in standard NPR1 binding assays (e.g., XX16, WW06, or WW03). The ability of a test antibody to inhibit the binding of anti-NPR1 antibodies to human NPR1 demonstrates that the test antibody can compete with that antibody for binding to human NPR1; such an antibody may, according to non-limiting theory, bind to the same or a related (e.g., a structurally similar or spatially proximal) epitope on human NPR1 as the antibody with which it competes. In a certain embodiment, the antibody that binds to the same epitope on human NPR1 as the antibodies described herein is a human antibody (e.g., a human monoclonal antibody or antigen binding fragment thereof). Such antibodies can be prepared and isolated as described herein.

Additional binding agents that bind to anti-NPR or target antibodies can be identified based on their ability to cross-compete (e.g., to competitively inhibit the binding of, in a statistically significant manner) with other antibodies described herein in standard binding assays. The ability of a test antibody to inhibit the binding of antibodies of the present disclosure to anti-NPR1 antibodies or antigen binding fragments thereof demonstrates that the test antibody can compete with binding agent for binding to an anti-NPR1 antibody; such a test antibody may, according to non-limiting theory, bind to the same or a related (e.g., a structurally similar or spatially proximal) epitope on the anti-NPR1 antibody as the binding agent with which it competes. In a certain embodiment, the test antibody that binds to the same epitope on the anti-NPR1 antibody as one of the binding agents described herein is a human antibody (e.g., a human monoclonal antibody or antigen binding fragment thereof). Such antibodies can be prepared and isolated as described herein.

Engineered and Modified Antibodies

An antibody of the disclosure can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is antibody binding region/paratope or CDR grafting. Because paratope sequences are responsible for most antibody-antigen interactions, it is possible to express recombinant antibodies that mimic the properties of specific naturally occurring antibodies by constructing expression vectors that include CDR/paratope sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad. See. U.S.A. 86:10029-10033; U.S. Pat. Nos. 5,225,539, and 5,530,101; 5,585,089; 5,693,762 and 6,180,370; the contents of each of which are herein incorporated by reference for this purpose).

Accordingly, another embodiment of the disclosure pertains to an isolated anti-NPR1 antibody, or an antigen-binding fragment thereof, comprising an antigen binding portion thereof, comprising a heavy chain variable region comprising the CDR sequences of an antibody or group of antibodies shown in Table 2. Thus, such antibodies contain the V_{H} and V_{L} CDR sequences of monoclonal antibodies, yet may contain different framework sequences from these antibodies.

Accordingly, another embodiment of the disclosure pertains to binding agents that specifically bind an isolated anti-NPR1 antibody, or an antigen-binding fragment thereof, comprising an antigen binding portion thereof, comprising a heavy chain variable region comprising the CDR sequences of an antibody or group of antibodies shown in Table 2.

Such framework sequences can be obtained from public DNA databases or published references that include germline antibody gene sequences. For example, germline DNA sequences for human heavy and light chain variable region genes can be found in the “VBase” human germline sequence database (available on the Internet at www [dot] mrc-cpe [dot] cam [dot] ac [dot] uk/vbase), as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. fol. Biol. 227:776-798; and Cox, J. P. L. et al., 1994 Eur. J Immunol. 24:827-836; the contents of each of which are herein incorporated by reference for this purpose.

An example of framework sequences for use in any of the antibodies or antigen binding fragments described herein are those that are structurally similar to the framework sequences used by selected antibodies described herein, e.g., consensus sequences and/or framework sequences used by the antibodies or antigen binding fragments. The V_{H} CDR1, 2 and 3 sequences, and the V_{L} CDR1, 2 and 3 sequences, can be grafted onto framework regions that have an identical sequence to that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370; the contents of each of which are herein incorporated by reference for this purpose).

Another type of variable region modification is to mutate amino acid residues within the V_{H} and/or V_{L} CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein. Conservative modifications (as discussed above) can also be introduced. The mutations may be amino acid substitutions, additions or deletions. Moreover, typically no more than one, two, three, four or five residues within a CDR region are altered.

Grafting Antigen Binding Domains into Alternative Frameworks or Scaffolds

A wide variety of antibody/immunoglobulin frameworks or scaffolds can be employed so long as the resulting polypeptide includes at least one binding region which specifically binds to an anti-NPR1 antibody or antigen binding fragment thereof, or to NPR1. Such frameworks or scaffolds include the 5 main idiotypes of human immunoglobulins, or fragments thereof (such as those described elsewhere herein), and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

In one aspect, the disclosure pertains to generating non-immunoglobulin based antibodies using non-immunoglobulin scaffolds onto which CDRs of the disclosure can be grafted. Known or future non-immunoglobulin frameworks and scaffolds may be employed, as long as they comprise a binding region specific for NPR1 (e.g., such as those described for an anti-NPR1 antibody described herein including, but not limited to, XX16, WW03, or WW06); or as long as they comprise a binding region specific for an anti-NPR1 antibody or antigen binding fragment thereof (e.g., such as those described for a binding agent that binds to such an anti-NPR1 antibody or binding fragment thereof (e.g., AA01-AA09). Such compounds are known herein as “polypeptides comprising a target-specific binding region”. Examples of non-immunoglobulin framework are further described in the sections below (camelid antibodies and non-antibody scaffold).

### Camelid Antibodies

Antibody proteins obtained from members of the camel and dromedary (Camelus bactrianus and Camelus dromaderius) family including new world members such as llama species (Lama paccos, Lama glama, and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals, see WO94/04678, the contents of which are herein incorporated by reference for this purpose.

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520; the contents of each of which are herein incorporated by reference for this purpose. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitated drug transport across the blood brain barrier, see US2004/0161738, the contents of which are herein incorporated by reference for this purpose. These features combined with the low antigenicity to humans indicate great therapeutic potential. Further, these molecules can be fully expressed in prokaryotic cells such as E. coli and may be expressed as functional fusion proteins with bacteriophage.

Accordingly, a feature of the present disclosure is a camelid antibody or nanobody having high affinity for NPR1; or having high affinity for an anti-NPR1 antibody. In one embodiment, the camelid antibody or nanobody is obtained by grafting the CDRs sequences of the heavy or light chain of the human antibodies of the disclosure into nanobody or single domain antibody framework sequences as described, for example, in WO94/04678 (the contents of which are herein incorporated by reference for this purpose).

### Framework or Fc Engineering

Engineered antibodies of the disclosure include those in which modifications have been made to framework residues within VH and/or VL, e.g., to improve one or more properties of the antibody. Typically such framework modifications are made to decrease the immunogenicity of the antibody. Antibodies of the disclosure may be modified in one or more ways, including each of the ways described herein.

For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies and additional modifications described herein are also intended to be encompassed herein.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in US2003/0153043, the contents of which are herein incorporated by reference for this purpose.

In addition or alternative to modifications made within the framework or CDR regions, antibodies described herein may be engineered to include modifications within the Fc region, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425, the contents of which are herein incorporated by reference for this purpose. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745, the contents of which are herein incorporated by reference for this purpose.

In another embodiment, the antibody is modified to increase its biological half-life. Various approaches are possible. For example, one or more of the following mutations can be introduced: T252L, T254S, T256F, as described in U.S. Pat. No. 6,277,375, the contents of which are herein incorporated by reference for this purpose. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022, the contents of each of which are herein incorporated by reference for this purpose.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, the contents of each of which are herein incorporated by reference for this purpose.

In order to minimize the ADCC activity of an antibody, specific mutations in the Fc region result in “Fc silent” antibodies that have minimal interaction with effector cells. In general, the “IgG Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody.

Silenced effector functions can be obtained by mutation in the Fc region of the antibodies. See, for example, LALA and N297A (Strohl, W., 2009, Curr. Opin. Biotechnol. vol. 20(6):685-691); and D265A (Baudino et al., 2008, J. Immunol. 181: 6664-69) see also Heusser et al., WO2012065950; the contents of each of which are herein incorporated by reference for this purpose. In particular, residues 234 and/or 235 may be mutated, optionally to alanine. Thus, in one embodiment, an antibody according to the disclosure has a mutation in the Fc region at one or both of amino acids 234 and 235. Such substitution of both amino acids 234 and 235 results in reduced ADCC activity. One example of such a mutation is the LALA mutant comprising L234A and L235A mutation in the IgG1 Fc amino acid sequence. Another example of a silent IgG1 antibody is the DAPA (D265A, P329A) mutation (U.S. Pat. No. 6,737,056, the contents of which are herein incorporated by reference for this purpose). Another silent IgG1 antibody comprises the N297A mutation, which results in aglycosylated/non-glycosylated antibodies. Fc silent antibodies result in no or low ADCC activity, meaning that an Fc silent antibody exhibits an ADCC activity that is below 50% specific cell lysis. No ADCC activity means that the Fc silent antibody exhibits an ADCC activity (specific cell lysis) that is below 1%.

In another embodiment, one or more amino acids selected from amino acid residues can be replaced with a different amino acid residue such that the antibody has altered Clq binding and/or reduced or abolished complement dependent cytotoxicity (CDC). This approach is described in further detail in U.S. Pat. No. 6,194,551, the contents of which are herein incorporated by reference for this purpose.

In another embodiment, one or more amino acid residues are altered to thereby alter the ability of the antibody to fix complement. This approach is described further in WO94/29351, the contents of which are herein incorporated by reference for this purpose.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in WO00/42072, the contents of which are herein incorporated by reference for this purpose. Moreover, the binding sites on human IgG1 for FcγRl, FcγRII, FcγRIII and FcRn have been mapped and variants with improved binding have been described (see Shields, R. L. et al., 2001 J. Biol. Chem. 276:6591-6604, the contents of which are herein incorporated by reference for this purpose).

In still another embodiment, the glycosylation of an antibody is modified. For example, an aglycoslated antibody can be made (i.e., the antibody lacks glycosylation). Glycosylation can be altered to, for example, increase the affinity of the antibody for “antigen”. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861, the contents of each of which are herein incorporated by reference for this purpose.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. For example, EP 1,176,195 (the contents of which are herein incorporated by reference for this purpose) describes a cell line with a functionally disrupted FUT8 gene, which encodes a fucosyl transferase, such that antibodies expressed in such a cell line exhibit hypofucosylation. WO03/035835 describes a variant CHO cell line, Lecl3 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). WO99/54342 describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180). The contents of each of the foregoing applications and references are herein incorporated by reference for this purpose

Another modification of the antibodies herein that is contemplated by the disclosure is pegylation. An antibody can be pegylated, for example, to increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See, for example, EP0154316 and EP0401384, the contents of each of which are herein incorporated by reference for this purpose.

Another modification of the antibodies that is contemplated herein is a conjugate or a protein fusion of at least the antigen-binding region of any of the antibodies described herein to serum protein, such as human serum albumin or a fragment thereof to increase half-life of the resulting molecule. Such an approach is described, for example, in EP0322094, the contents of which are herein incorporated by reference for this purpose.

Another possibility is a fusion of at least the antigen-binding region of the antibody of the disclosure to proteins capable of binding to serum proteins, such human serum albumin to increase half-life of the resulting molecule. Such approach is described, for example, in EP0486525, the contents of which are herein incorporated by reference for this purpose.

Nucleic Acid Molecules Encoding Antibodies Described Herein

Another aspect of the present document pertains to nucleic acid molecules that encode the antibodies described herein. The term “nucleic acid” is used herein interchangeably with the term “polynucleotide,” and refers to deoxyribonucleotides or ribonucleotides and polymers thereof in either single- or double-stranded form. The term encompasses nucleic acids containing known nucleotide analogs or modified backbone residues or linkages, which are synthetic, naturally-occurring, and non-naturally-occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). In some embodiments, the nucleic acid may be an mRNA.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (See: Batzer et al., Nucleic Acids Res 1991; 25(19):5081; Ohtsuka et al., J Biol Chem 1985; 260(5):2605-8; Rossolini et al., Mol Cell Probes 1994; 8(2):91-8; the contents of each of which are herein incorporated by reference for this purpose).

Provided herein are exemplary full length heavy and light chain nucleotide sequences of anti-NPR1 antibodies. In some embodiments, the nucleic acid molecules are one or more of those identified in Table 2, e.g., those encoding an anti-NPR1 antibody or antigen binding fragment thereof. In some other embodiments, the nucleic acid molecules described herein comprise nucleotide sequences that are substantially identical (e.g., at least 80%, 85%, 90%, 91%, 92%, 93%. 94%. 95%. 96%, 97%. 98%, or 99%) to the nucleotide sequences of those identified in Table 2. When expressed from appropriate expression vectors, polypeptides encoded by these polynucleotides are capable of binding to a NPR1 protein (e.g., human NPR1).

Also provided herein are polynucleotides which encode at least one CDR region, and usually all three CDR regions, from the heavy and/or light chain of an anti-NPR1 antibody or antigen binding fragment of the disclosure. Further provided herein are polynucleotides which encode all or substantially all of the variable region sequence of the heavy chain and/or the light chain of an exemplary anti-NPR1 antibody or antigen binding fragment of the disclosure. Because of the degeneracy of the genetic code, a variety of nucleic acid sequences will encode each of the immunoglobulin amino acid sequences.

In some embodiments, the nucleic acid molecules disclosed herein encode both a variable region and a constant region of an antibody. In some embodiments, the nucleic acid molecules disclosed herein comprise nucleotides encoding a full-length heavy chain sequence that is substantially identical (e.g., at least 80%, 85%, 90%, 91%, 92%, 93%, 94%, 95%, 96%, 97%, 98%, or 99%) to the heavy chain sequence of one of the antibodies described herein including those in Table 2. In some embodiments, the nucleic acid molecules disclosed herein comprise nucleotides encoding a full-length light chain sequence that is substantially identical (e.g., at least 80%, 85%, 90%, 91%, 92%, 93%, 94%, 95%, 96%, 97%, 98%, or 99%) to the light chain sequence of one of the antibodies described herein including those in Table 2.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. 1987 Current Protocols in Molecular Biology, Greene Publishing and Wiley Interscience, New York, the contents of which are herein incorporated by reference for this purpose. A nucleic acid of the disclosure can be, for example, DNA or RNA and may or may not contain intronic sequences. In an embodiment, the nucleic acid is a cDNA molecule. The nucleic acid may be present in a vector such as a phage display vector, or in a recombinant plasmid vector.

Nucleic acids of any of the antibodies described herein can be obtained using standard molecular biology techniques. For antibodies expressed by hybridomas (e.g., hybridomas prepared from transgenic mice carrying human immunoglobulin genes as described further herein), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from various phage clones that are members of the library.

The polynucleotide sequences can be produced by de novo solid-phase DNA synthesis or by PCR mutagenesis of an existing sequence (e.g., sequences as described herein in, for example, Table 2). Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68: 109, 1979; the diethylphosphoramidite method of Beaucage et al., Tetra. Lett., 22: 1859, 1981; and the solid support method of U.S. Pat. No. 4,458,066 (the contents of each of which are herein incorporated by reference for this purpose). Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif, 1990; Mattila et al., Nucleic Acids Res. 19:967, 1991; and Eckert et al., PCR Methods and Applications 1:17, 1991.

Once DNA fragments encoding VH and VL segments are obtained, these DNA fragments can be further manipulated by standard recombinant DNA techniques, for example to convert the variable region genes to full-length antibody chain genes, to Fab fragment genes or to an scFv gene. In these manipulations, a VL- or VH-encoding DNA fragment is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., el al., 1991 Sequences of Proteins of Immunological Interest, Fifth Edition, U.S. Department of Health and Human Services, NIH Publication No. 91-3242, the contents of which are herein incorporated by reference for this purpose) and DNA fragments encompassing these regions can be obtained by standard PCR amplification. The heavy chain constant region can be an IgG1, IgG2, IgG3, IgG4, IgA, IgE, IgM, or IgD constant region. In some embodiments, the heavy chain constant region is an IgG1 isotype. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as to a Fab light chain gene) by operatively linking the VL-encoding DNA to another DNA molecule encoding the light chain constant region, CL. The sequences of human light chain constant region genes are known (see, e.g., Kabat, E. A., et al., 1991 Sequences of Proteins of Immunological Interest, Fifth Edition, U.S. Department of Health and Human Services, NIH Publication No. 91-3242, the contents of which are herein incorporated by reference for this purpose) and DNA fragments encompassing these regions can be obtained by standard PCR amplification. The light chain constant region can be a kappa or a lambda constant region.

To create an scFv gene, the VH- and VL-encoding DNA fragments are operatively linked to another fragment encoding a flexible linker, such that the VH and VL sequences can be expressed as a contiguous single-chain protein, with the VL and VH regions joined by the flexible linker (see e.g., Bird et al., 1988 Science 242:423-426; Huston et al., 1988 Proc. Natl. Acad. Sci. USA 85:5879-5883; McCafferty et al., 1990 Nature 348:552-554; the contents of each of which are herein incorporated by reference for this purpose).

Vectors

Various expression vectors can be employed to express the polynucleotides encoding the antibody of the disclosure or antigen-binding fragment thereof. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet. 15:345, 1997, the contents of which are herein incorporated by reference for this purpose). For example, nonviral vectors useful for expression of the polynucleotides and polypeptides of the multispecific antibody of the disclosure or domains thereof in mammalian (e.g., human) cells include pThioHis A, B and C, pcDNA3.1/His, pEBVHis A, B and C, (Invitrogen, San Diego, Calif), MPS V vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68: 143, 1992, the contents of each of which are herein incorporated by reference for this purpose.

The choice of expression vector depends on the intended host cells in which the vector is to be expressed. Expression vectors for mammalian host cells can include expression control sequences, such as an origin of replication, a promoter, and an enhancer (see, e.g., Queen, et al., Immunol. Rev. 89:49-68, 1986, the contents of which are herein incorporated by reference for this purpose), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP poIIII promoter, the constitutive MPS V promoter, the tetracycline-inducible CMV promoter (such as the human immediate-early CMV promoter), the constitutive CMV promoter, and known promoter-enhancer combinations.

Cultures of transformed organisms can be expanded under non-inducing conditions without biasing the population for coding sequences whose expression products are better tolerated by the host cells. In addition to promoters, other regulatory elements may also be required or desired for efficient expression of the antibody of the disclosure or fragments thereof. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20: 125, 1994; and Bittner et al., Meth. Enzymol., 153:516, 1987; the contents of each of which are herein incorporated by reference for this purpose). For example, the SV40 enhancer or CMV enhancer may be used to increase expression in mammalian host cells.

Accordingly, the disclosure provides a cloning or expression vector comprising one or more of the nucleic acid sequences of any of the antibodies described herein; e.g., the antibodies shown in Table 2. Furthermore, the disclosure provides a cloning or expression vector comprising a nucleic acid encoding one or more of the nucleotide sequences shown in Table 2.

Host Cells

For expression of the light and heavy chains, the expression vector or expression vectors encoding the heavy and light chains may be transferred into a host cell by standard techniques.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra, the contents of which are herein incorporated by reference for this purpose). Other methods include, e.g., electroporation, calcium phosphate treatment, liposome-mediated transformation, injection and microinjection, ballistic methods, virosomes, immunoliposomes, polycatiomnucleic acid conjugates, naked DNA, artificial virions, fusion to the herpes virus structural protein VP22 (Elliot and O'Hare, Cell 88:223, 1997, the contents of which are herein incorporated by reference for this purpose), agent-enhanced uptake of DNA, and ex vivo transduction.

It is theoretically possible to express the antibodies of the disclosure in either prokaryotic or eukaryotic host cells. Expression of antibodies in eukaryotic cells, in particular mammalian host cells, is discussed because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R., 1985 Immunology Today 6:12-13, the contents of which are herein incorporated by reference for this purpose).

For long-term, high-yield production of recombinant proteins, stable expression will often be desired. For example, cell lines which stably express the antibodies or antigen-binding fragments thereof of the disclosure can be prepared using expression vectors of the disclosure which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type. The present disclosure thus provides a method of producing the antibodies or antigen-binding fragments of the disclosure, wherein said method comprises the step of culturing a host cell comprising a nucleic acid encoding the antibodies or antigen-binding fragments.

In some embodiments, mammalian host cells are used to express and produce any of the antibodies described herein; e.g., the binding agents that bind to the anti-NPR1 antibodies and antigen binding fragments described herein; or the anti-NPR1 antibodies or antigen binding fragments described herein. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed including the CHO cell lines, various COS cell lines, HeLa cells, myeloma cell lines, transformed B-cells, and hybridomas. Exemplary host cells include but are not limited to Chinese hamster ovary (CHO) cells, human embryonic kidney (HEK) cells (e.g., HEK293, HEK293T, HEK293F), monkey kidney (COS) cells (e.g., COS-1, COS-7), baby hamster kidney (BHK) cells (e.g., BHK-21), African green monkey kidney cells (e.g. BSC-1), HeLa cells, human hepatocellular carcinoma cells (e.g., Hep G2), myeloma cells (e.g., NS0, 653, SP2/0), lymphoma cells, oocyte cells, and cells from a transgenic animal (e.g., mammary epithelial cells), or any derivative, immortalized, or transformed cell thereof. In particular, for use with NS0 myeloma cells, another expression system is the GS gene expression system shown in WO87/04462, WO89/01036 and EP0338841, the contents of each of which are herein incorporated by reference for this purpose. When recombinant expression vectors encoding antibody nucleic acid are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods. Such purified antibodies of the disclosure may be used for any purpose including, but not limited to, the methods and uses described herein, and/or as part of a pharmaceutical composition as described herein.

In a further alternative, the host cell may be a yeast or a filamentous fungi engineered for mammalian-like glycosylation pattern, and capable for producing antibodies lacking fucose as glycosylation pattern (see, for example, EP1297172, the contents of which are herein incorporated by reference for this purpose).

Accordingly, the disclosure provides a host cell comprising one or more of the vectors, or nucleic acid sequences of the disclosure described above.

Generation of Monoclonal Antibodies of the Disclosure

Monoclonal antibodies (mAbs) can be produced by a variety of techniques, including conventional monoclonal antibody methodology e.g., the somatic cell hybridization technique of Kohler and Milstein, 1975 Nature 256: 495, the contents of which are herein incorporated by reference for this purpose. Many techniques for producing monoclonal antibody can be employed e.g., viral or oncogenic transformation of B lymphocytes.

Hybridomas may be prepared using, for example, the murine system. Immunization protocols and isolation of immunized splenocytes for fusion may be performed according to any appropriate procedure. Chimeric or humanized antibodies can be prepared based on the sequence of a murine monoclonal antibody prepared as described herein. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using any known methods (see e.g., U.S. Pat. No. 4,816,567, the contents of which are herein incorporated by reference for this purpose). To create a humanized antibody, the murine CDR regions can be inserted into a human framework using any known methods. See e.g., U.S. Pat. Nos. 5,225,539, and 5,530,101; 5,585,089; 5,693,762 and 6,180,370, the contents of each of which are herein incorporated by reference for this purpose.

Human monoclonal antibodies can also be generated using transgenic or transchromosomic mice carrying parts of the human immune system rather than the mouse system. These transgenic and transchromosomic mice include mice referred to herein as HuMAb Mice® and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., 1994 Nature 368(6474): 856-859, the contents of which are herein incorporated by reference for this purpose). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N. Y. Acad. Sci. 764:536-546; the contents of each of which are herein incorporated by reference for this purpose). The preparation and use of HuMAb mice*, and the genomic modifications carried by such mice, is further described in Taylor, L. et al., 1992 Nucleic Acids Research 20:6287-6295; Chen, J. et al., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of each of which are hereby incorporated by reference for this purpose. See further, U.S. Pat. Nos. 5,545,806; 5,569,825; 5,625,126; 5,633,425; 5,789,650; 5,877,397; 5,661,016; 5,814,318; 5,874,299; and 5,770,429; 5,545,807; WO92/103918, WO93/12227, WO94/25585, WO97/113852, WO98/24884 and WO99/45962; and WO01/14424; the contents of each of which are hereby incorporated by reference for this purpose.

In another embodiment, human antibodies can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in WO02/43478, the contents of which are hereby incorporated by reference for this purpose.

Still further, alternative transgenic animal systems expressing human immunoglobulin genes are available and can be used to raise any of the antibodies described herein. For example, an alternative transgenic system referred to as the Xenomouse (Abgenix, Inc.) can be used. Such mice are described in, e.g., U.S. Pat. Nos. 5,939,598; 6,075,181; 6,114,598; 6,150,584 and 6,162,963, the contents of each of which are hereby incorporated by reference for this purpose.

Moreover, alternative transchromosomic animal systems expressing human immunoglobulin genes are available in the art and can be used to raise any of the antibodies described herein. For example, mice carrying both a human heavy chain transchromosome and a human light chain tranchromosome, referred to as “TC mice” can be used; such mice are described in Tomizuka et al., 2000 Proc. Natl. Acad. Sci. USA 97:722-727, the contents of which are hereby incorporated by reference for this purpose. Furthermore, cows carrying human heavy and light chain transchromosomes have been described in the art (Kuroiwa et al., 2002 Nature Biotechnology 20:889-894, the contents of which are hereby incorporated by reference for this purpose) and can be used to raise any of the antibodies described herein.

Human monoclonal antibodies of the disclosure can also be prepared using phage display methods for screening libraries of human immunoglobulin genes. Such phage display methods for isolating human antibodies are established and/or described in the examples below. See for example: U.S. Pat. Nos. 5,223,409; 5,403,484; and 5,571,698; 5,427,908 and 5,580,717; 5,969,108 and 6,172,197; and 5,885,793; 6,521,404; 6,544,731; 6,555,313; 6,582,915 and 6,593,081, the contents of each of which are hereby incorporated by reference for this purpose.

Human antibodies or antigen binding fragments as described herein can also be prepared using SCID mice into which human immune cells have been reconstituted such that a human antibody response can be generated upon immunization. Such mice are described in, for example, U.S. Pat. Nos. 5,476,996 and 5,698,767, the contents of each of which are hereby incorporated by reference for this purpose.

Any of the antibodies described herein may be prepared by any of the methods described herein, or any other known method.

Generation of Hybridomas Producing Antibodies or Antigen Binding Fragments Described Herein

To generate hybridomas producing the antibodies or antigen binding fragments described herein, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Cells are plated at approximately 2×145 in flat bottom microtiter plates, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin, and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify antibodies or antigen binding fragments thereof, selected hybridomas can be grown in two-liter spinner-flasks for antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The antibodies or antigen binding fragments can be aliquoted and stored at −80° C.

Hybridomas producing the antibodies or antigen binding fragments as described herein may be produced, for example, using the methods described herein.

Generation of Transfectomas Producing Antibodies or Antigen Binding Fragments

Antibodies or antigen binding fragments described herein can also be produced in a host cell transfectoma using, for example, a combination of suitable recombinant DNA techniques and gene transfection methods (e.g., Morrison, S. (1985) Science 229:1202, the contents of which are incorporated herein by reference for this purpose).

For example, to express the antibodies or antigen-binding fragments thereof, nucleic acids (e.g., DNAs) encoding partial or full-length light and heavy chains can be obtained by standard molecular biology techniques (e.g., PCR amplification or cDNA cloning using a hybridoma that expresses the antibody of interest) and the nucleic acids (e.g., DNAs) can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or both genes may be inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{L} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the disclosure carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology; Methods in Enzymology 185, Academic Press, San Diego, C A 1990, the contents of which are incorporated herein by reference for this purpose). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1 (Takebe, Y. et al., 1988 Mol. Cell. Biol. 8:466-472, the contents of which are incorporated herein by reference for this purpose).

In addition to the antibody chain genes and regulatory sequences, the recombinant expression vectors described herein may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, the contents of each of which are incorporated herein by reference for this purpose). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr− host cells with methotrexate selection/amplification) and the neo gene (for G418 selection).

For expression of the light and heavy chains, the expression vector(s) encoding the heavy and light chains is or are transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the disclosure in either prokaryotic or eukaryotic host cells. Expression of antibodies in eukaryotic cells, in particular mammalian host cells, is discussed because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R., 1985 Immunology Today 6:12-13, the contents of which are incorporated herein by reference for this purpose).

Mammalian host cells for expressing the antibodies or antigen binding fragments described herein are described elsewhere herein. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods. Accordingly, the disclosure provides a process for the production of one or more of the anti-NPR1 antibodies or antigen-binding fragments thereof described herein, comprising culturing a host cell of the disclosure and isolating the antibody or antigen-binding fragment thereof.

Uses and Methods of Treatment

Methods of Treatment using Anti-NPR1 Antibodies and Antigen Binding Fragments

Provided herein are methods of treating a disease associated with NPR1 loss of function by using the anti-NPR1 antibodies or antigen binding fragments thereof disclosed herein (e.g., an antibody or group of antibodies as defined in Table 2; e.g., the target antigens or antigen binding fragments thereof). In some embodiments, the target or anti-NPR1 antibody or antigen binding fragment thereof may be selected from WW01_LALA, WW03_LALA, WW05_LALA, WW06_LALA, XX01_LALA, XX01_DAPA, XX01_N30S_DAPA, XX03_LALA, XX04_LALA, XX06_LALA, XX06_DAPA, XX07_LALA, XX08_LALA, XX08_DAPA, XX08_N30S_DAPA, XX08_N30Q_DAPA, XX09_LALA, XX11_LALA, XX12_LALA, XX13_LALA, XX14_LALA, XX15_LALA, XX15_DAPA, XX16_LALA, XX16_DAPA, XX17_LALA, XX17_DAPA, XX18_LALA, XX18_DAPA, XX19_LALA, XX19_DAPA, XX20_LALA, XX20_DAPA, YY01_LALA, YY02_LALA, YY03_LALA, YY04_LALA, YY05_LALA, YY06_LALA, YY07_LALA, ZZ05_LALA, ZZ12 LALA. ZZ13 LALA. ZZ14 LALA, ZZ15 LALA. ZZ16 LALA, and ZZ17 LALA.

In some embodiments, the target or anti-NPR1 antibody or antigen binding fragment thereof may be selected from WW01_LALA, WW03_LALA, XX01_LALA, XX01_DAPA, XX01_N30S_DAPA, XX03_LALA, XX04_LALA, XX06_LALA, XX06_DAPA, XX07_LALA, XX08_LALA, XX08_DAPA, XX08_N30S_DAPA, XX08_N30Q_DAPA, XX09_LALA, XX11_LALA, XX12_LALA, XX13_LALA, XX14_LALA, XX15_LALA, XX15_DAPA, XX16_LALA, XX16_DAPA, XX17_LALA, XX17_DAPA, XX18_LALA, XX18_DAPA, XX19_LALA, XX19_DAPA, XX20_LALA, XX20_DAPA, YY01_LALA, YY03_LALA, YY04_LALA, ZZ12_LALA, and ZZ13_LALA. In some embodiments, the target or anti-NPR1 antibody or antigen binding fragment thereof may be selected from WW05_LALA, WW06_LALA, YY05_LALA, YY06_LALA, YY07_LALA, ZZ05_LALA, ZZ14_LALA, and ZZ16_LALA. In some embodiments, the target or anti-NPR1 antibody or antigen binding fragment thereof may be XX16_DAPA. In some embodiments, the target or anti-NPR1 antibody or antigen binding fragment thereof may be XX16_LALA. In some embodiments, the target or anti-NPR1 antibody or antigen binding fragment thereof may be XX16, XX16_DAPA, or XX16_LALA.

In some embodiments, the disease associated with NPR1 loss of function is a cardiovascular disorder. In some embodiments, the cardiovascular disorder is selected from: hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, and myocardial infarction (MI). In some embodiments, the disease associated with NPR1 loss of function is heart failure, hypertrophic cardiomyopathy (HCM), hypertension, preeclampsia, asthma, glaucoma, or cytokine release syndrome. In some embodiments, the heart failure is selected from a heart failure with reduced ejection fraction (HFrEF), heart failure with preserved ejection fraction (HFpEF), heart failure after acute myocardial infarct, or acute decompensated heart failure. In some embodiments, the hypertrophic cardiomyopathy is ventricular hypertrophy. In some embodiments, the hypertension is selected from resistant hypertension, hypertensive heart disease, pulmonary hypertension, isolated systolic hypertension, and pulmonary arterial hypertension. In some embodiments, the hypertension is selected from resistant hypertension and hypertensive heart disease.

In some embodiments, the disease associated with NPR1 loss of function is a kidney disorder. In some embodiments, the kidney disorder is selected from: diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD).

NPR1-related disorders also include any other disorders which are directly or indirectly associated with aberrant NPR1 activity and/or expression. Provided herein are also methods of treating a NPR1 related disorder directly or indirectly associated with aberrant NPR1 activity and/or expression by using the target or anti-NPR1 antibodies or antigen binding fragments disclosed herein (e.g., from Table 2, such as XX16_DAPA or XX16_LALA).

In some embodiments, the present disclosure provides methods of treating an undesirable condition, disease, or disorder associated with natriuretic peptide receptor activity in a subject in need thereof, the method comprising administering to the subject a therapeutically effective amount of an target or anti-NPR1 antibody or antigen binding fragment disclosed herein. In some embodiments, the present disclosure provides a use of a target or anti-NPR1 antibody or antigen binding fragment disclosed herein for treatment of an undesirable condition, disease or disorder associated with natriuretic peptide receptor activity in a subject in need thereof. In some embodiments, the present disclosure provides a target or anti-NPR1 antibody or antigen binding fragment disclosed herein for use in a method for treating an undesirable condition, disease or disorder associated with natriuretic peptide receptor activity. In some embodiments, the present disclosure provides a target or anti-NPR1 antibody or antigen binding fragment disclosed herein for use in manufacturing a medicament for treating an undesirable condition, disease or disorder associated with natriuretic peptide receptor activity. Such conditions, diseases and disorders include, but are not limited to, cardiovascular disorders (e.g., hypertension, peripheral vascular disease, heart failure (including but not limited to heart failure with reduced ejection fraction (HFrEF), heart failure with preserved ejection fraction (HFpEF), heart failure after acute myocardial infarct, or acute decompensated heart failure), coronary artery disease (CAD), ischemic heart disease (IHD), mitral stenosis and regurgitation, angina, hypertrophic cardiomyopathy (e.g., ventricular hypertrophy), diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI)), hypertension (e.g., resistant hypertension, hypertensive heart disease, pulmonary hypertension, isolated systolic hypertension, or pulmonary arterial hypertension), preeclampsia, asthma, glaucoma, cytokine release syndrome, and/or a kidney disorder (e.g., diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)).

In some embodiments, such methods include administering to a subject in need of treatment a therapeutically effective amount of a target or anti-NPR1 antibody or antigen-binding fragment thereof that specifically binds to the same epitope as one of the target or anti-NPR1 antibodies described herein. For example, such methods include administering to a subject in need of treatment a therapeutically effective amount of a target or anti-NPR1 antibody or antigen-binding fragment thereof that specifically binds to the same epitope as XX16. In another embodiment, such methods include administering to a subject in need of treatment a therapeutically effective amount of a target or anti-NPR1 antibody or antigen-binding fragment thereof that specifically binds to the same epitope as WW03. In another embodiment, such methods include administering to a subject in need of treatment a therapeutically effective amount of a target or anti-NPR1 antibody or antigen-binding fragment thereof that specifically binds to the same epitope as WW06.

All the aforementioned embodiments for the methods of protection and treatment according to the present invention are equally applicable to
the use of any one of the target or anti-NPR1 antibodies or antigen binding fragments as described herein for the manufacture of a medicament for use according to the present invention,
the use of any one of the target or anti-NPR1 antibodies or antigen binding fragments described herein according to the present invention,
any one of the target or anti-NPR1 antibodies or antigen binding fragments described herein for use according to the present invention,
the pharmaceutical compositions comprising any one of the target or anti-NPR1 antibodies or antigen binding fragments described herein for the use according to the present invention,
the use of the pharmaceutical compositions comprising any one of the target or anti-NPR1 antibodies or antigen binding fragments described herein according to the present invention, and
the use of the pharmaceutical compositions comprising any one of the target or anti-NPR1 antibodies or antigen binding fragments described herein for the manufacture of a medicament for use according to the present invention.

Methods of Treatment Using Binding Agents Targeting Anti-NPR1 Antibodies and Antigen Binding Fragments

It is known that the ANP/NPR1 system regulates blood pressure; and it is known that natriuretic peptides elicit their physiological responses through the synthesis of cyclic GMP (cGMP). See, e.g., Potter et al., Endocrine Reviews; Vol. 27, No. 1, pp 47-72 (2006); and Kamiya et al., Hear and Vessels; Vol. 35, No. 1; pp 59-68 (2019). With an increase in ANP (e.g., through agonism of NPR1, such as through use of an agonistic anti-NPR1 antibody or antigen binding fragment thereof), a subject may experience hypotension.

Therefore, also provided herein are methods of treating a disease or disorder associated with NPR1 agonism (e.g., NPR1 agonism caused by an NPR1 agonist agent, such as an agonistic anti-NPR1 antibody or antigen binding fragment thereof) in a subject in need thereof. Such a disease or disorder (e.g., hypotension) may be treated by administering an binding agent that specifically binds to the NPR1 agonist agent (e.g., the anti-NPR1 antibody or antigen binding fragment thereof). Provided herein are methods of treating hypotension (e.g., hypotension caused by an NPR1 agonist agent, such as an agonistic anti-NPR1 antibody or antigen binding fragment thereof) in a subject in need thereof, e.g., in a subject who has been administered an anti-NPR1 antibody or antigen binding fragment thereof. Provided herein are methods of reversing the effects on cGMP of an NPR1 agonist agent (e.g., an agonistic anti-NPR1 antibody or antigen binding fragment thereof) in a subject in need thereof, e.g., in a subject who has been administered an anti-NPR1 antibody or antigen binding fragment thereof.

Provided herein are methods of treating a disease or disorder associated with NPR1 agonism (e.g., hypotension) by administering a binding agent that specifically binds to one or more of the target anti-NPR1 antibodies or antigen binding fragments thereof described herein (e.g., an antibody or group of antibodies as defined in Table 2). In some embodiments, the anti-NPR1 antibody or antigen binding fragment thereof may be selected from WW01_LALA, WW03_LALA, WW05_LALA, WW06_LALA, XX01_LALA, XX01_DAPA, XX01_N30S_DAPA, XX03_LALA, XX04_LALA, XX06_LALA, XX06_DAPA, XX07_LALA, XX08_LALA, XX08_DAPA, XX08_N30S_DAPA, XX08_N30Q_DAPA, XX09_LALA, XX11_LALA, XX12_LALA, XX13_LALA, XX14_LALA, XX15_LALA, XX15_DAPA, XX16_LALA, XX16_DAPA, XX17_LALA, XX17_DAPA, XX18_LALA, XX18_DAPA, XX19_LALA, XX19_DAPA, XX20_LALA, XX20_DAPA, YY01_LALA, YY02_LALA, YY03_LALA, YY04_LALA, YY05_LALA, YY06_LALA, YY07_LALA, ZZ05_LALA, ZZ12_LALA, ZZ13_LALA, ZZ14_LALA, ZZ15_LALA, ZZ16_LALA, and ZZ17_LALA.

In some embodiments, the target anti-NPR1 antibody or antigen binding fragment thereof may be selected from WW01_LALA, WW03_LALA, XX01_LALA, XX01_DAPA, XX01_N30S_DAPA, XX03_LALA, XX04_LALA, XX06_LALA, XX06_DAPA, XX07_LALA, XX08_LALA, XX08_DAPA, XX08_N30S_DAPA, XX08_N30Q_DAPA, XX09_LALA, XX11_LALA, XX12_LALA, XX13_LALA, XX14_LALA, XX15_LALA, XX15_DAPA, XX16_LALA, XX16_DAPA, XX17_LALA, XX17_DAPA, XX18_LALA, XX18_DAPA, XX19_LALA, XX19_DAPA, XX20_LALA, XX20_DAPA, YY01_LALA, YY03_LALA, YY04_LALA, ZZ12_LALA, and ZZ13_LALA. In some embodiments, target anti-NPR1 the antibody or antigen binding fragment thereof may be selected from WW05_LALA, WW06_LALA, YY05_LALA, YY06_LALA, YY07_LALA, ZZ05_LALA, ZZ14_LALA, and ZZ16_LALA. In some embodiments, the target anti-NPR1 antibody or antigen binding fragment thereof may be XX16_DAPA. In some embodiments, the target anti-NPR1 antibody or antigen binding fragment thereof may be XX16_LALA.

In some embodiments, the target anti-NPR1 antibody or antigen fragment thereof may have been administered in order to treat a cardiovascular disorder. In some embodiments, the cardiovascular disorder is selected from: hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, and myocardial infarction (MI). In some embodiments, the target anti-NPR1 antibody or antigen fragment thereof may have been administered in order to treat heart failure, hypertrophic cardiomyopathy (HCM), hypertension, preeclampsia, asthma, glaucoma, or cytokine release syndrome. In some embodiments, the heart failure is selected from a heart failure with reduced ejection fraction (HFrEF), heart failure with preserved ejection fraction (HFpEF), heart failure after acute myocardial infarct, or acute decompensated heart failure. In some embodiments, the hypertrophic cardiomyopathy is ventricular hypertrophy. In some embodiments, the hypertension is selected from resistant hypertension, hypertensive heart disease, pulmonary hypertension, isolated systolic hypertension, and pulmonary arterial hypertension. In some embodiments, the hypertension is selected from resistant hypertension and hypertensive heart disease.

In some embodiments, the target anti-NPR1 antibody or antigen fragment thereof may have been administered in order to treat a kidney disorder. In some embodiments, the kidney disorder is selected from: diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD).

In some embodiments, the target anti-NPR1 antibody or antigen fragment thereof may have been administered in order to treat any other NPR1-related disorders which are directly or indirectly associated with aberrant NPR1 activity and/or expression. Provided herein are also methods of treating a disease or disorder directly or indirectly associated with aberrant NPR1 activity and/or expression by administering an anti-NPR1 antibodies or antigen binding fragments described herein (e.g., from Table 2, such as XX16_DAPA or XX16_LALA).

In some embodiments, the present disclosure provides methods of treating an undesirable condition, disease, or disorder associated with agonism of natriuretic peptide receptor activity in a subject in need thereof, the method comprising administering to the subject a therapeutically effective amount of a binding agent that specifically binds to an anti-NPR1 (or target) antibody or antigen binding fragment described herein. In some embodiments, the present document provides a use of a binding agent that binds to an anti-NPR1 (target) antibody or antigen binding fragment disclosed herein for treatment of an undesirable condition, disease or disorder associated with heightened natriuretic peptide receptor activity in a subject in need thereof. In some embodiments, the present disclosure provides a binding agent that specifically binds to an anti-NPR1 (target) antibody or antigen binding fragment disclosed herein for use in a method for treating an undesirable condition, disease or disorder associated with an increase in natriuretic peptide receptor activity associated with administration of the anti-NPR1 (target) antibody or antigen binding fragment thereof. In some embodiments, the present disclosure provides a binding agent that specifically binds an anti-NPR1 antibody or antigen binding fragment disclosed herein for use in manufacturing a medicament for treating an undesirable condition, disease or disorder associated with agonism of natriuretic peptide receptor activity (e.g., associated with administration of the anti-NPR1 (target) antibody or antigen binding fragment thereof). Such a condition, disease, or disorder may be hypotension. The anti-NPR1 antibody may have been administered to treat one or more disorders described elsewhere herein.

In some embodiments, such methods include administering to a subject in need of treatment a therapeutically effective amount of a binding agent that specifically binds to an anti-NPR1 (target) antibody or antigen-binding fragment thereof that specifically binds to the same epitope as one of the anti-NPR1 antibodies described herein. For example, such methods include administering to a subject in need of treatment a therapeutically effective amount of a binding agent that specifically binds to an anti-NPR1 antibody or antigen-binding fragment thereof that specifically binds to the same epitope as XX16. In another embodiment, such methods include administering to a subject in need of treatment a therapeutically effective amount of a binding agent that specifically binds to an anti-NPR1 (target) antibody or antigen-binding fragment thereof that specifically binds to the same epitope as WW03. In another embodiment, such methods include administering to a subject in need of treatment a therapeutically effective amount of a binding agent that specifically binds an anti-NPR1 (target) antibody or antigen-binding fragment thereof that specifically binds to the same epitope as WW06.

All the aforementioned embodiments for the methods of protection and treatment according to the present invention are equally applicable to
the use of any one of the binding agents, reversal agents, or anti-idiotype antibodies that binds to an anti-NPR1 (target) antibodies or antigen binding fragments as described herein for the manufacture of a medicament for use according to the present invention,
the use of any one of the binding agents, reversal agents, or anti-idiotype antibodies that binds to an anti-NPR1 (target) antibodies or antigen binding fragments described herein according to the present invention,
any one of the binding agents, reversal agents, or anti-idiotype antibodies that binds to an anti-NPR1 (target) antibodies or antigen binding fragments described herein for use according to the present invention,
the pharmaceutical compositions comprising any one of the binding agents, reversal agents, or anti-idiotype antibodies that binds to an anti-NPR1 (target) antibodies or antigen binding fragments described herein for the use according to the present invention,
the use of the pharmaceutical compositions comprising any one of the binding agents, reversal agents, or anti-idiotype antibodies that binds to an anti-NPR1 (target) antibodies or antigen binding fragments described herein according to the present invention, and
the use of the pharmaceutical compositions comprising any one of the binding agents, reversal agents, or anti-idiotype antibodies that binds to an anti-NPR1 (target) antibodies or antigen binding fragments described herein for the manufacture of a medicament for use according to the present invention.

Combination Therapies

NPR1 agonist treatments as described herein might be combined with other treatment partners or therapeutic agents such as the current standard of care for a disease associated with NPR1 loss of function, e.g., the current standard of care for one or more of the diseases or disorders discussed herein associated with NPR1. For example, the NPR1 antibodies or an antigen-binding fragment thereof described herein can be combined with one or more of an ACE (angiotensin-converting-enzyme) inhibitor, an angiotensin receptor blocker (ARB), a neprilysin inhibitor, a beta blocker, a diuretic, a calcium channel blocker, a cardiac glycoside, a sodium-glucose co-transporter 2 inhibitor (SGLT2i), or combinations thereof. As a non-limiting set of examples, the NPR1 antibody or antigen binding from may be combined with an additional therapeutic agent selected from enalapril, benazepril, captopril, fosinopril, lisinopril, moexipril, perindopril, quinapril, ramipril, trandolapril, valsartan, azilsartan, candesartan, eprosartan, irbesartan, losartan, olmesartan, telmisartan, sacubitril, bisoprolol, carvedilol, propanolol, metoprolol, metoprolol tartrate, metoprolol succinate, thiazide diuretics, loop diuretics, potassium-sparing diuretics, amlodipine, clevidipine, diltiazem, felodipine, isradipine, nicardipine, nifedipine, nisoldipine, verapamil, a digitalis glycoside, canagliflozin, dapagliflozin, empagliflozin, ertugliflozin, and combinations thereof. Exemplary diuretics and digitalis glycosides include, but are not limited to, chlorothiazide, chlorthalidone, hydrochlorothiazide, indapamide, metolazone, bumetanide, ethacrynic acid, furosemide, torsemide, amiloride, eplerenone, spironolactonem, triamterene, digoxin, and combinations thereof. In some embodiments, the NPR1 antibodies or an antigen-binding fragment thereof described herein may be combined with an angiotensin receptor-neprilysin inhibitor (ARNi) such as a combination of sacubitril and valsartan (e.g., Entresto®). In some embodiments, the NPR1 antibodies or an antigen-binding fragment thereof described herein can be combined with one or more of a corticosteroid (e.g., an inhaled corticosteroid such as fluticasone, budesonide, mometasone, beclomethasone, ciclesonide, or fluticasone furoate; or an oral or intravenous corticosteroid such as prednisone or methylprednisolone), a leukotriene modifier (e.g., montelukast, zafirlukast, or zileuton), a bronchodilator (e.g., a long-acting beta agonist (e.g., salmeterol or formoterol), a short-acting beta agonist (e.g., albuterol or levalbuterol), theophylline or ipratropium), or combinations thereof (e.g., a combination of fluticasone and salmeterol, a combination of budesonide and formoterol, or a combination of formoterol and mometasone). In some embodiments, the NPR1 antibodies or an antigen-binding fragment thereof described herein can be combined with one or more of a beta-adrenoceptor antagonist (e.g., timolol, levobunolol, metipranolol, carteolol, or betaxolol), a carbonic anhydrase inhibitor (e.g., acetazolamide, dorzolamide, brinzolamide, or methazolamide), an alpha 2-adrenoceptor agonist (e.g., brimonidine or apraclonidine), a parasympathomimetic (e.g., cholinomimetics like pilocarpine), a prostaglandin analog (e.g., latanoprost, latanoprostene bunod, travoprost, bimatoprost, or tafluprost), a rho kinase inhibitor (e.g., netarsudil or ripasudil), or combinations thereof (e.g., a combination of rho kinase inhibitor and latanoprost).

Accordingly, the methods of treating a disease associated with NPR1 loss of function described herein can further include administering a second agent to the subject in need of treatment as described above.

If a reversal of the NPR1 agonist treatment is desired (e.g., because of an emergent disease or disorder such as hypotension), one or more of the reversal agents described herein (e.g., one or more of the binding agents described herein that binds to an anti-NPR1 antibody or antigen binding fragment thereof) may be administered to a subject in need of such treatment (e.g., a subject who is experiencing hypotension after the administration of the anti-NPR1 antibody or antigen binding fragment thereof). If such a reversal is desired, the method may further comprise additionally administering one of the following to the subject: (i) fluid infusion; and/or (ii) one or more vasopressors. In some embodiments, the fluid infusion may be used to raise blood pressure through an increase in blood volume. In some embodiments, the fluid infusion is a crystalloid IV fluid infusion. In some embodiments, the fluid infusion is a saline infusion. In some embodiments, the fluid infusion is an infusion of one or more of the following: lactated Ringer's solution, hypertonic saline, and isotonic saline. In some embodiments, any known vasopressor may be used, or more that one vasopressor may be used. In some embodiments, one or more vasopressors is selected from phenylephrine, norepinephrine, epinephrine, and vasopressin.

In some embodiments, the reversal of the NPR1 agonist treatment (e.g., in addition to administering the reversal agent and/or administering a fluid infusion) additionally comprises halting treatment with an additional therapeutic agent (e.g., an additional therapeutic agent that had been administered with the NPR1 agonist, e.g., the anti-NPR1 antibody or antigen binding fragment). Such an additional therapeutic agent (e.g., the additional therapeutic agent that had been originally administered to a patient in need thereof along with or in combination with an agonist anti-NPR1 antibody or antigen binding fragment thereof) may be an anti-hypertensive agent, a beta blocker, and/or a diuretic.

The term “combination” refers to either a fixed combination in one dosage unit form, or a combined administration where an antibody or antigen-binding fragment thereof described herein (e.g., an anti-NPR1 antibody or antigen binding fragment thereof; or a binding agent that binds to a target anti-NPR1 antibody or antigen binding fragment thereof) and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration and/or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., a compound of the present invention and a combination partner, are both administered to a patient simultaneously in the form of a single entity or dosage. The term “non-fixed combination” means that the therapeutic agents, e.g., a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g., synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

Pharmaceutical Compositions, Dosages, and Methods of Administration

Also provided herein are compositions, e.g., pharmaceutical compositions, for use in treatment of an NPR1-associated disease. Such compositions include one or more anti-NPR1 antibodies or an antigen-binding fragment thereof as described herein and may include a pharmaceutically acceptable carrier. Such compositions can further include another agent, e.g., a current standard of care for the disease to be treated.

Also provided herein are compositions, e.g., pharmaceutical compositions, for use in treatment of a disease or disorder associated with NPR1 agonism (e.g., hypotension). Such compositions include one or more binding agents which specifically bind to an anti-NPR1 antibody or an antigen-binding fragment thereof as described herein and may include a pharmaceutically acceptable carrier. Such compositions can further include another agent, e.g., a current standard of care for the disease to be treated.

Pharmaceutical compositions typically include a pharmaceutically acceptable carrier. As used herein the term “pharmaceutically acceptable cater” refers to a carrier or a diluent that does not cause significant irritation to a subject and does not abrogate the biological activity and properties of the administered antibody or antigen binding fragment and/or any additional therapeutic agent in the composition. Pharmaceutically acceptable carriers may enhance or stabilize the composition or can be used to facilitate preparation of the composition. Pharmaceutically acceptable carriers may include saline, solvents, dispersion media, coatings, antibacterial and antifungal agents, isotonic and absorption delaying agents, and the like, compatible with pharmaceutical administration. An adjuvant may also be included in any of these formulations. Pharmaceutical compositions are typically formulated to be compatible with its intended route of administration. Examples of routes of administration include parenteral (e.g., intravenous, intraarterial, intraperitoneal), oral, intracranial, intrathecal, or intranasal (e.g., inhalation), intradermal, subcutaneous, or transmucosal administration. In some embodiments, the pharmaceutical compositions are formulated to deliver antibodies or antigen-binding fragments thereof to cross the blood-brain barrier. The phrases “physiologically acceptable carrier” and “pharmaceutically acceptable carrier” may be used interchangeably.

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

A pharmaceutical composition of the present disclosure can be administered by a variety of methods known in the art. The route and/or mode of administration may vary depending upon the desired results. In some embodiments, the administration is intravitreal, intravenous, intramuscular, intraperitoneal, or subcutaneous. The pharmaceutically acceptable carrier should be suitable for intravitreal, intravenous, intramuscular, subcutaneous, parenteral, spinal, or epidermal administration (e.g., by injection or infusion). Depending on the route of administration, the active compound(s), i.e., the antibody or antigen binding fragment and optionally the additional therapeutic agent, may be coated in a material to protect the compound(s) from the action of acids and other natural conditions that may inactivate the compound(s).

Typically, a therapeutically effective dose or efficacious dose of the antibodies or antigen binding fragments is employed in the pharmaceutical compositions of the present disclosure. The antibodies or antigen binding fragments may be formulated into pharmaceutically acceptable dosage forms by conventional methods known to those of skill in the art.

Methods of formulating suitable pharmaceutical compositions are known in the art, see, e.g., Remington: The Science and Practice of Pharmacy. 21^{st} ed., 2005; and the books in the series Drugs and the Pharmaceutical Sciences: a Series of Textbooks and Monographs (Dekker, NY), the contents of each of which are incorporated by reference herein for this purpose. For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Dosage regimens for any of the antibodies and antigen binding fragments described herein with or without an additional therapeutic agent may be adjusted to provide the optimum desired response (e.g., a therapeutic response). For example, a single bolus of one or both agents may be administered at one time, several divided doses may be administered over a predetermined period of time, or the dose of one or both agents may be proportionally reduced or increased as indicated by the exigencies of the therapeutic situation. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The skilled artisan (such as a medical doctor) will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Dosage regimens for any of the antibodies and antigen binding fragments described herein alone or in combination with an additional therapeutic agent may be adjusted to provide the optimum desired response (e.g., a therapeutic response). For example, a single bolus of one or both agents may be administered at one time, several divided doses may be administered over a predetermined period of time, or the dose of one or both agents may be proportionally reduced or increased as indicated by the exigencies of the therapeutic situation. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Dosage values for compositions comprising any antibody or antigen binding fragment described herein, and/or any additional therapeutic agent(s), may be selected based on the unique characteristics of the active compound(s), and the particular therapeutic effect to be achieved. A physician or veterinarian can start doses of the antibodies of the disclosure employed in the pharmaceutical composition at levels lower than that required to achieve the desired therapeutic effect and gradually increase the dosage until the desired effect is achieved. In general, effective doses of the compositions of the present disclosure, for the treatment of obesity or another disorder described herein may vary depending upon many different factors, including means of administration, target site, physiological state of the patient, whether the patient is human or an animal, other medications administered, and whether treatment is prophylactic or therapeutic. The selected dosage level may also depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. Treatment dosages may be titrated to optimize safety and efficacy.

Kits

Also provided herein are kits including one or more of the compositions provided herein (e.g., a binding agent that specifically binds to an anti-NPR1 antibody or antigen binding fragment thereof described in Table 2; with or without the anti-NPR1 antibody or antigen binding fragment thereof) and instructions for use. Instructions for use can include instructions for diagnosis or treatment of a disease or disorder associated with NPR1 agonism (e.g., hypotension; e.g., low blood pressure); and may additionally provide instructions for the binding agent (which may also be called, e.g., a reversal agent as described elsewhere herein). Instructions for use can also include instructions for diagnosis or treatment of an NPR1-associated disease. Kits as provided herein may be used in accordance with any of the methods described herein. Those skilled in the art will be aware of other suitable uses for kits provided herein, and will be able to employ the kits for such uses. Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

The disclosure is further illustrated by the following examples and claims, which are illustrative and are not meant to be further limiting. One skilled in the art will recognize many methods and materials similar or equivalent to those described herein, which could be used in the practice of the described compositions and methods. Such equivalents are within the scope of the present disclosure and claims. The contents of all references, including issued patents and published patent applications, cited throughout this application are hereby incorporated by reference.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

This disclosure provides anti-NPR1 antibodies that specifically bind and activate NPR1, e.g., antibodies and antigen binding fragments that (i) bind to NPR1; and (ii) activate NPR1 in the absence of ANP. Antibodies that specifically bind and activate NPR1 could have different possible modes of action: (1) the antibody induces a conformational change within the NPR1 monomers to activate the receptor; (2) the antibody directly mimics the structure and function of the natural ligand ANP and activates the receptor by binding in the ANP binding pocket of NPR1; or (3) the antibody stabilizes the preformed functionally active complex of hNPR1 and ANP (NPR1-ANP-complex).

### Example 1: HuCAL® Phage Library Panning and Screening

For the selection of NPR1-specific antibodies covering the described different methods of action, 13 different panning strategies were applied (see Table 3). Ten strategies were performed exclusively on protein (strategies 1-6 and 10-13). In addition, three differential cell pannings were performed (strategies 7-9). In total, four panning strategies (strategies 3 and 11-13) aimed for the enrichment of ANP competing antibodies (elution with ANP, pre-adsorption of phage on NPR1-ANP-complexes, and anti-idiotype pannings on murine anti-ANP antibodies).

TABLE 3
Overview of HuCAL ® panning strategies

| Strategy | 1^{st} round | 2^{nd} round | 3^{rd} round | comments |
|---|---|---|---|---|
| 1 | hNPR1 | hNPR1 | hNPR1 | Solution panning |
| | solution | solution | solution | with human |
| | | | | antigen only |
| 2 | hNPR1 | hNPR1 | hNPR1 | Fc capture panning |
| | capture | capture | capture | with human |
| | | | | antigen only |
| 3 | hNPR1 | hNPR1 | hNPR1 | Solution panning |
| | solution | solution + | solution + | aiming for |
| | | ANP | ANP | enrichment of |
| | | elution | elution | ANP competitors |
| 4 | hNPR1 | hNPR1 | hNPR1 | Solution panning |
| | solution | solution + | solution + | aiming for |
| | | pH 5.8 | pH 5.8 | enrichment of |
| | | elution | elution | pH dependent |
| | | | | binders |
| 5 | hNPR1 | rNPR1 | hNPR1 | Solution/Fc |
| | solution | capture | solution | capture panning |
| | | | | aiming for rat |
| | | | | crossreactivity |
| 6 | rNPR1 | hNPR1 | rNPR1 | Fc capture panning |
| | capture | capture | capture | aiming for rat |
| | | | | crossreactivity |
| 7 | hNPR1 | CHO-K1 | rNPR1 | Fc capture/cell |
| | capture | NPR1 | capture | panning aiming |
| | | cells | | for rat |
| | | | | crossreactivity |
| 8 | hNPR1 | CHO-K1 | hNPR1 | Solution/cell |
| | solution | NPR1 | solution | panning with |
| | | cells | | human |
| | | | | antigen only |
| 9 | CHO-K1 | hNPR1 | CHO-K1 | Solution/ |
| | NPR1 cells | solution | NPR1 | cell panning |
| | | | cells | with human |
| | | | | antigen only |
| 10 | hNPR1- | rNPR1- | hNPR1- | Solution panning |
| | ANP- | ANP- | ANP- | aiming for |
| | complex | complex | complex | enrichment of |
| | solution | capture | solution | NPR-ANP |
| | | | | complex |
| | | | | stabilizers and rat |
| | | | | crossreactivity |
| 11 | pre- | pre- | pre- | Solution panning |
| | adsorption | adsorption | adsorption | aiming for |
| | on NPR1- | on NPR1- | on NPR1- | enrichment of |
| | ANP | ANP | ANP | ANP competitors |
| | complex, | complex, | complex, | |
| | save the | save the | save the | |
| | unbound | unbound | unbound | |
| | phage still | phage still | phage still | |
| | in solution, | in solution, | in solution, | |
| | bind to | bind to | bind to | |
| | hNPR1 in | hNPR1 in | hNPR1 in | |
| | solution | solution | solution | |
| | without | without | without | |
| | ANP and | ANP and | ANP and | |
| | capture | capture | capture | |
| | NPR1/ | NPR1/ | NPR1/ | |
| | phage | phage | phage | |
| | complexes | complexes | complexes | |
| 12 | mouse | hNPR1 | mouse | Fc capture/cell |
| | anti- | solution | anti- | panning with |
| | ANP-mAb | | ANP-mAb | anti-ANP mAb |
| | capture | | capture | |
| 13 | mouse | mouse | mouse | Fc capture panning |
| | anti-ANP | anti- | anti- | with anti-ANP |
| | mix of | ANP mix | ANP mix | mAb aiming for |
| | two | of two | of two | enrichment of |
| | mAbs | mAbs | mAbs | ANP competitors |
| | capture | capture + | capture + | |
| | | ANP | ANP | |
| | | elution | elution | |

For Fc capture panning, NPR1-hFc was immobilized on a 96-well plate via an appropriate capture antibody (a goat or mouse anti-human Fc antibody). The antigen was immobilized in an appropriate number of wells of a 96-well plate and wells were subsequently blocked prior to the addition of phage-antibodies. In parallel to well preparation, phage-antibodies were blocked. During blocking of phage, additional blocking reagents were added to the blocking buffer to avoid selection of antibodies against the hFc-tag or the capture antibody (goat or mouse y globulin). Following the blocking procedure, two pre-adsorption steps on human γ globulin and on the counter-target hNPR3-hFc were performed to avoid selection of antibodies against the Fc-tag or the counter-target. The pre-blocked and pre-adsorbed phage mix was added to each well with immobilized NPR1-hFc and the phage-antibodies were allowed to bind to the antigen. Intensive washing ensured removal of non-specifically bound phage, followed by elution of specifically bound phage. The second and third round of solid phase panning was performed according to the protocol of the first panning round. Amounts of antigen were decreased and washing conditions with increased stringency were applied.

For solution panning, NPR1 was biotinylated and the retained activity of biotinylated NPR1 for ANP binding was confirmed. During solution panning, the Fab displaying phage and the biotinylated NPR1-hFc were incubated in solution, which facilitated the accessibility of the antigen by the phage. An appropriate amount of Streptavidin beads was blocked and, in parallel, an appropriate amount of phage-antibodies was blocked. During blocking of phage, human γ globulin, the counter-target hNPR3-hFc and the Flag-TEV linker peptide were added to the blocking buffer to avoid selection of antibodies against the hFc-tag, the counter-target, or the linker peptide. For removal of Streptavidin-, Biotin-, or bead-binding phage, pre-adsorption steps of blocked phage particles were performed using blocked Streptavidin beads with and without coupled biotinylated irrelevant antigen. Subsequently, biotinylated NPR1-hFc/NPR1-hFc-ANP-complex was added to the pre-adsorbed and blocked phage particles and the phage-antibodies were allowed to bind to the antigen in solution. For enrichment of antibody phage binding to the ANP-binding site of NPR1 (ANP competitive antibodies) the pre-formed NPR1-ANP-complex was added to the phage blocking solution or the ANP peptide was used for elution of the bound phage. Thereby, the ANP peptide was used at least in 250-fold molar excess to the NPR1 antigen or the NPR1 expressing cells. The phage-antigen complexes were captured using blocked Streptavidin beads and phage particles bound to the Streptavidin beads were collected with a magnetic separator. Phage bound nonspecifically were washed off by several washing steps. Specifically bound phage were eluted from Streptavidin beads. The eluate was transferred to an E. coli culture for phage infection. The second and third round of bead-based solution panning was performed according to the protocol of the first panning round. Amounts of antigen were decreased and washing conditions with increased stringency were applied.

For whole cell panning, an appropriate amount of phage-antibodies was blocked. During blocking of phage, counter-target hNPR3-hFc was added to the blocking buffer to avoid selection of antibodies against the counter-target. In parallel, an appropriate amount of target cells expressing NPR1 and an appropriate amount of adsorption cells without expression of antigen (parental cells) per phage pool were blocked. The blocked target cells were spun down, resuspended in the pre-blocked phage particles and the phage-antibodies were allowed to bind to the NPR1 presented on the cell. The phage-cell complexes were washed several times. For enrichment of antibody phage binding to the ANP-binding site of NPR1 (ANP competitive antibodies) the pre-formed NPR1-ANP-complex was added to the phage blocking solution or the ANP peptide was used for elution of the bound phage. Thereby, the ANP peptide was used at least in 250-fold molar excess to the NPR1 antigen or the NPR1 expressing cells. Specifically bound phage were eluted from target cells. After centrifugation, the supernatant (eluate) was applied to adsorption cells for removal of phage binding to cell surface molecules other than the target antigen (post-adsorption). The final supernatant was transferred to an E. coli culture for phage infection. The second and third round of the whole cell panning were performed according to the protocol of the first panning round. Washing conditions with increased stringency were applied.

The outputs of the panning rounds were subsequently subcloned into bacterial expression vectors and bacterial lysates (BEL) were used for primary and secondary screening. The outputs were analyzed for binding to human and rat NPR1 during the primary screening (ELISA-based). Clones binding to human NPR3 were deselected. Secondary screening was performed on hNPR1 expressing CHO-K1 cells. Further screenings regarding ANP competition and binding solely in presence of ANP were performed. Approximately 1700 clones fulfilled the screening selection criteria and 760 clones were selected for sequencing. The sequencing of 760 clones resulted in 210 HCDR3 unique hits, whose binding properties are summarized in Table 4. Of these clones, 72 demonstrated significant ANP competition, while 7 clones bound only in presence of ANP.

TABLE 4
Binding properties of 210 HCDR3 unique hits (HuCAL ®)

| | | | Binding to | | |
|---|---|---|---|---|---|
| | | | | | hNPR1 |
| | | | | | expr. |
| Number of HCDR3 unique candidates | | | hNPR1 | rNPR1 | cells |
| 210 of which | 47 human/ | 28 human/rat cross | Yes | Yes | Yes |
| 7 clones | rat cross | reactive cell binders | | | |
| only bind | reactive | 19 human/rat cross | Yes | Yes | No |
| in presence | | reactive (not binding | | | |
| of ANP | | to cells) | | | |
| | 156 | 53 human specific | Yes | No | Yes |
| | human | cell binders | | | |
| | specific | 103 human specific | Yes | No | No |
| | | (not binding to cells) | | | |

### Example 2: Antibody Reformatting, Expression, and Purification

After confirmation of binding, the VH and VL domains of the 210 HCDR3 unique clones were subcloned into a vector with a human IgG constant region. 180 of the 210 clones were selected for expression and 166 of the 180 passed the production quality control. They were characterized in regard to binding to relevant cell lines and functional activity. 40 of the 166 candidates were then selected for exploratory scale production, and 31 of these candidates were characterized in detail as shown below with respect to binding to relevant antigens and cell lines, ANP competition, and functionality in a cell based cGMP production assay.

For production of the IgG candidates, eukaryotic HKB11 cells were transfected with mammalian expression vector DNA encoding both heavy and light chains of IgG. Cell culture supernatants were harvested at appropriate times and subjected to Protein A affinity chromatography. If needed, a second purification step was performed to remove aggregates. Buffer exchange was performed to 1× Dulbecco's PBS (pH 7.2) and samples were sterile filtered (0.2 μm pore size).

### Example 3: Antigen Binding, ANP Competition and Cellular cGMP Production—HuCAL® Candidates

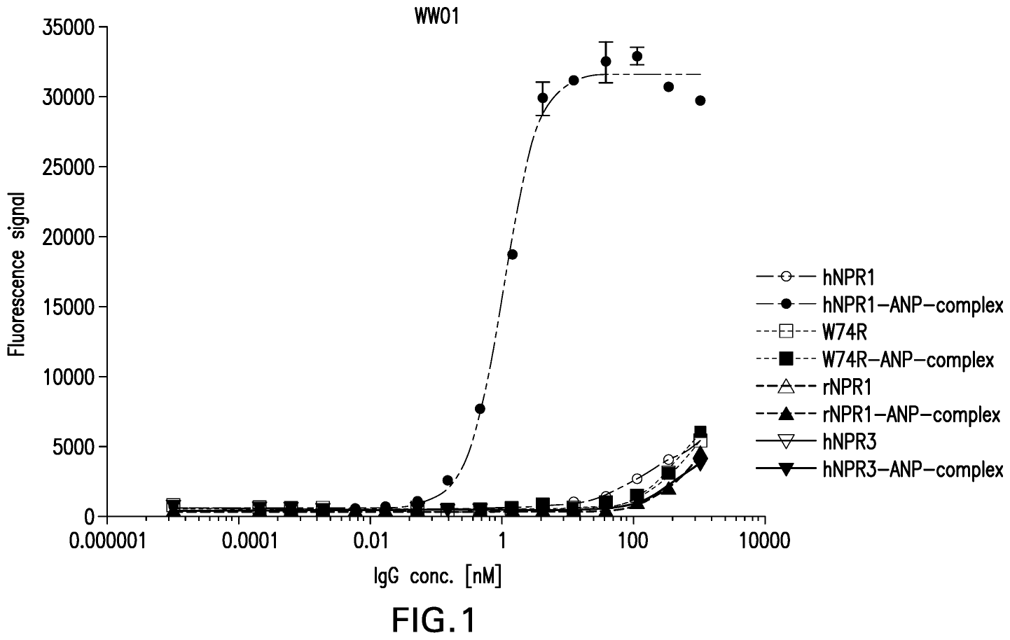

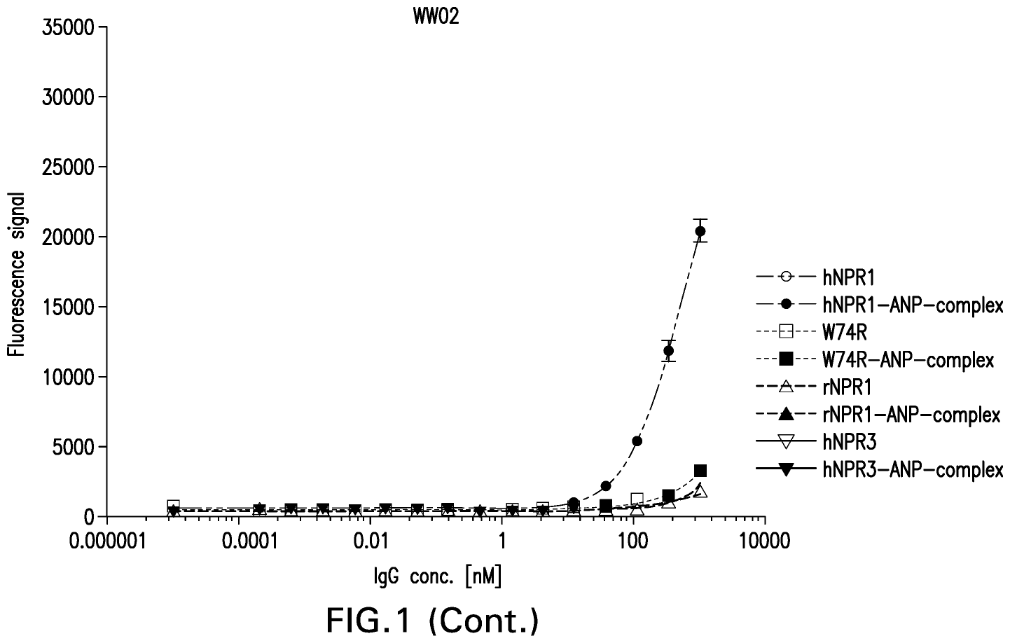

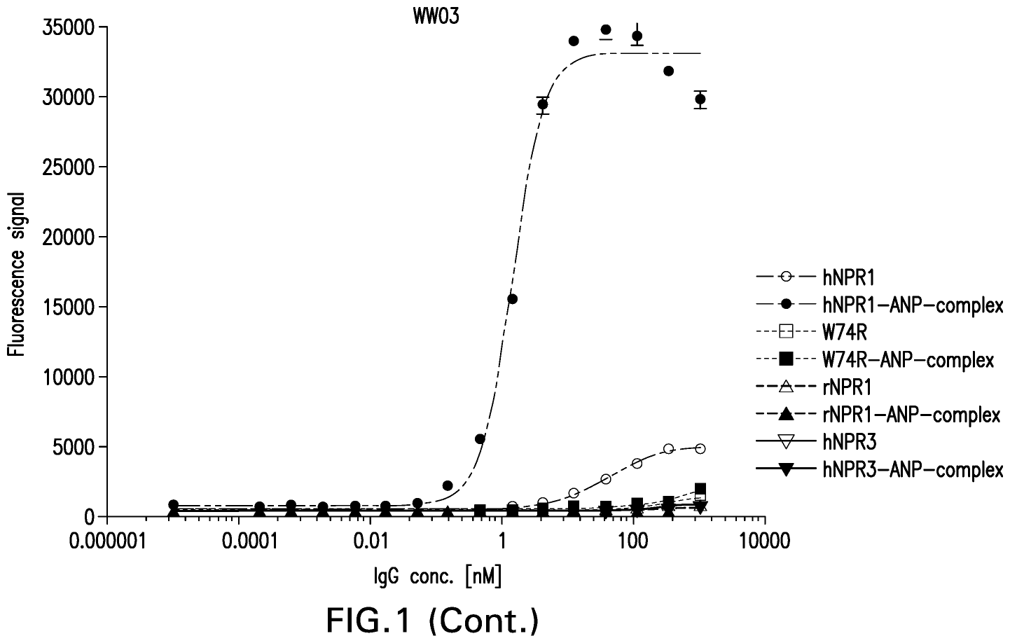

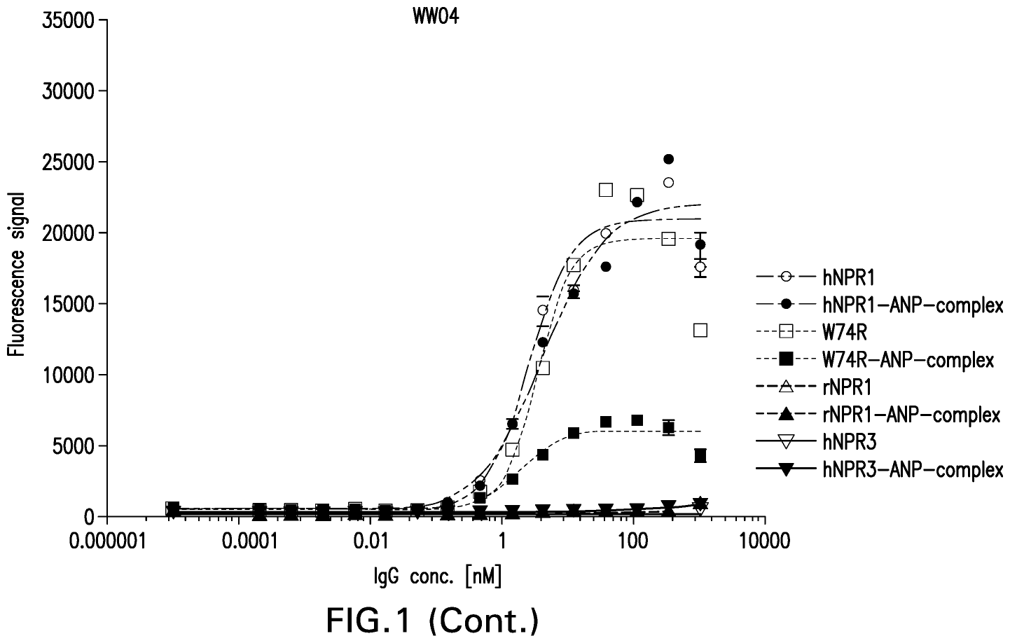

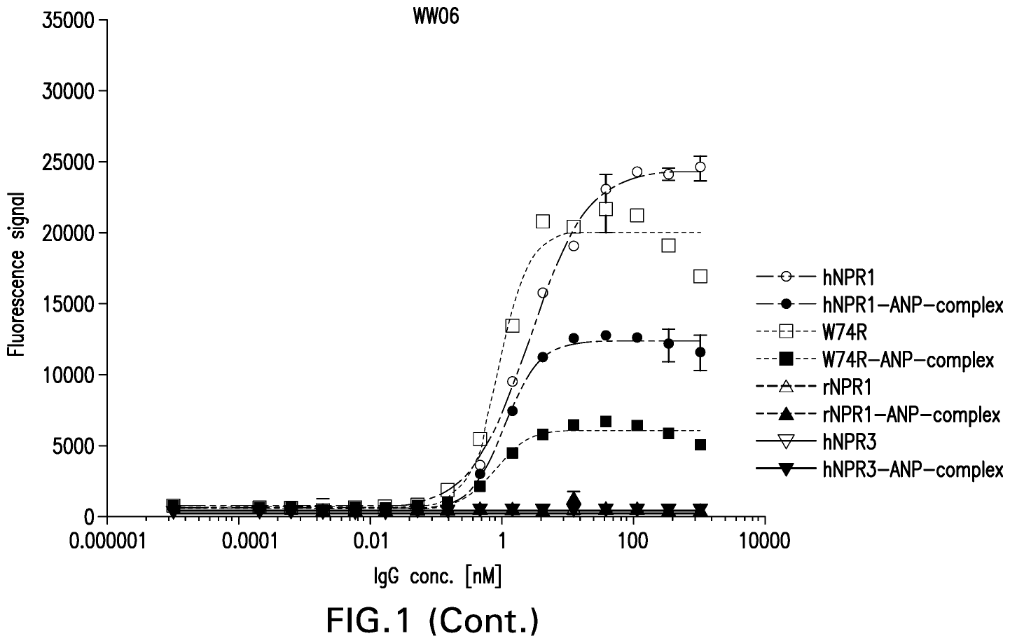

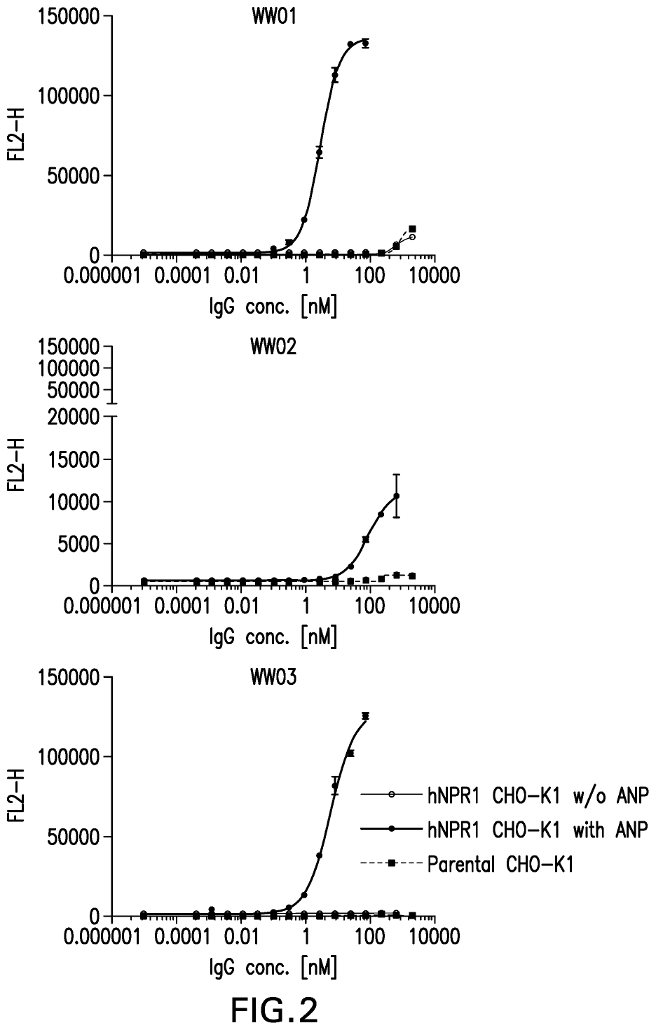

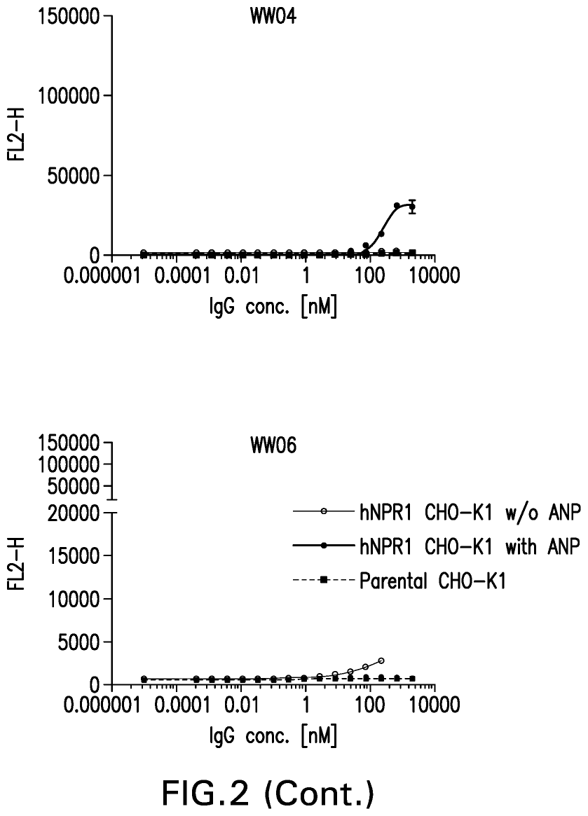

The 31 IgGs which passed the exploratory scale production quality control were tested via ELISA for binding to the following antigens: human NPR1, constitutively active human NPR1 mutant (W74R), rat NPR1, and human NPR3 (counter target). The clones were also tested by flow cytometry for binding to human NPR1 expressing CHO K1 cells in the absence and presence of ANP and on parental CHO K1 cells. The binding properties of the five functional candidates are shown in FIG. 1 and FIG. 2.

The same 31 IgGs were tested for ANP competition using a Fluorescence Resonance Energy Transfer (FRET)-based assay in which the NPR1-specific antibodies competed with ANP for binding to NPR1. In this FRET based assay (see FIG. 3), Eu-labeled Streptavidin (for measurement of IgGs) or Eu-labeled anti-hFc antibody (for measurement of FabCys) was used as an energy donor, while Cy5-labeled ANP was used as an acceptor (for all measurements). The resulting fluorescent signal was decreased by ANP-competitive antibodies. The assay was performed as follows: antibodies were mixed with NPR1 and incubated for 5 minutes at room temperature. After addition of the Eu-labeled donor and incubation for 30 minutes at room temperature, the Cy5-ANP solution was added. After a further 60-minute incubation, readout was performed using a TECAN Infinite M1000 Pro using an excitation wavelength of 317 nm and an emission wavelength of 665 nm. Percentage of ANP competition was calculated according to the following formulae:

Ratio*=[(A₆₆₅ nm/A₆₂₀ nm)*10⁴]

Ratio=(Ratio*−Ratio_{neg})

Competition %=[100−(Ratio/(Ratioₚₒₛ/100))]
Ratio_{neg}: mean Ratio* data values of control without NPR1
Ratioₚₒₛ: mean Ratio data values of control without agonist (reaction buffer)

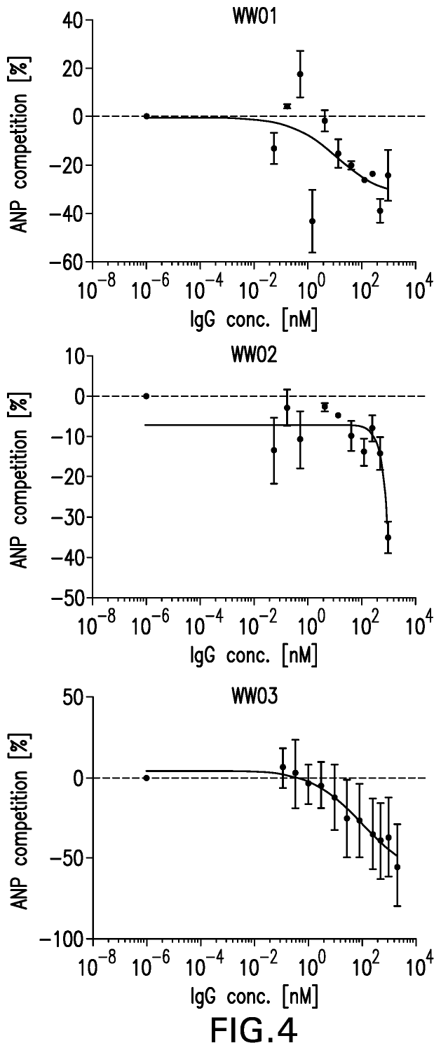

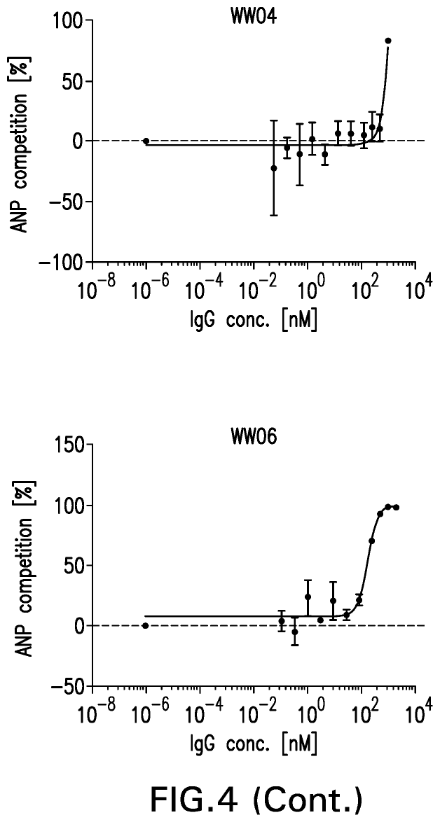

15 of the 31 IgGs were ANP competitive, but only two of these candidates showed functionality in the cGMP assay (WW04 and WW06). The other three functional candidates WW01, WW02 and WW03 demonstrated a “negative” ANP competition in this assay indicating the stabilization of the NPR1-ANP-complex. The FRET assay results for the five functional candidates are depicted in FIG. 4.

Additionally, as discussed above, the 31 IgGs were tested for their functional activity in a cellular cGMP production assay using human NPR1 expressing CHO-K1 cells. For the functional characterization of the selected antibodies the production of cyclic guanosine 3′,5′-cyclic monophosphate (cGMP) upon binding to and stimulation of NPR1 expressed on the cell surface of CHO-K1 cells was monitored. Cellular cGMP is a major second messenger that mediates cell activities and is synthesized by activated NPR1 triggered by ANP or NPR1-specific antibodies. Therefore, a commercial assay kit was used (Cisbio Bioassays CisBio HTRF Assay Kit CisBio (Cat. #62GM2PEB)). The assay was performed according to manufacturer's instructions with minor deviations. In brief, cells were adjusted to 1×10⁵ cells/mL, 20 μL/well were seeded in 96 well microtiter plates and were incubated overnight. After addition of 10 μL/well of the antibodies in different concentrations, the plate was incubated for 30 min at 37° C. to allow for cGMP production. In parallel, a standard curve using a calibrator (contained in the kit) was generated. The cells were lysed and a mix of cGMP-d2 and anti-cGMP-Cryptate was added and incubated for 1h at room temperature. The readout was performed using a Tecan M1000 Pro using an excitation wavelength of 317 nm and an emission wavelength of 665 nm. cGMP concentration (Delta F [%]) was calculated according to the following formulae:

Ratio=[(A₆₆₅ₙₘ/B₆₂₀ₙₘ)*10⁴]

Mean Ratio=(Σratios/2)

CV=[(Std deviation/Mean ratio)*100]

Delta F=[((Calibrator or sample Ratio−Ratio_{neg})/Ratio_{neg})*100]
Ratio_{neg}: negative control

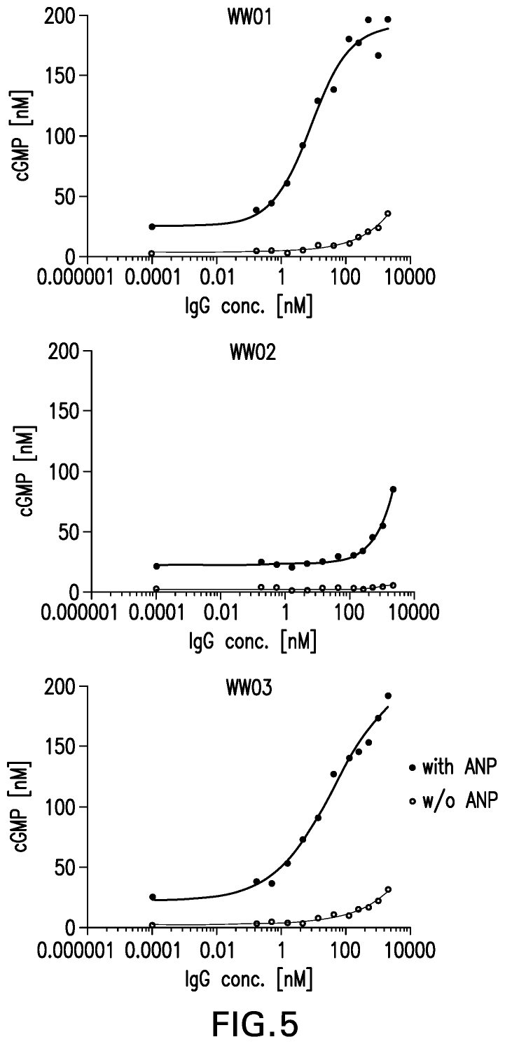

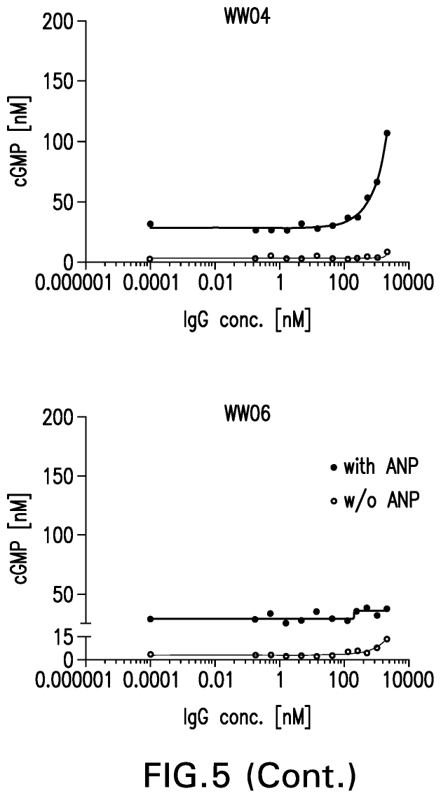

Five candidates with significant functional activity were identified using the cellular cGMP assay: WW01, WW02, WW03, WW04, and WW06. These five candidates were functionally active and could be assigned to different methods of action. WW01, WW02, and WW03 were able to stabilize the NPR1-ANP-complex, while WW06 was determined to be ANP competitive. These candidates were all derived from initial panning codes 10 and 11 (aiming for method of action 2 or 3). The results of the assay for the cellular production of cGMP in the absence or presence of 0.075 nM ANP induced by the five functional candidates (IgG format) are shown in FIG. 5.

### Example 4: Production and Characterization of HuCAL® Candidates in FabCys Format

The functional clones were also tested for functionality in FabCys format. Eukaryotic HKB11 cells were transfected with mammalian expression vector DNA encoding both heavy and light chains of disulfide-bridged FabCys. Cell culture supernatants were harvested at appropriate times and subjected to metal ion affinity chromatography using a liquid handling station. Buffer exchange was performed to 1× Dulbecco's PBS (pH 7.2) and samples were sterile filtered (0.2 μm pore size).

The five functional candidates WW01, WW02, WW03, WW04 and WW06 were additionally analyzed with regard to their monovalent affinities for human and rat NPR1 and the counter-target human NPR3 in absence and presence of ANP in monovalent FabCys format. The results of the affinity determination, epitope binning, and cGMP assay are summarized in Table 5.

TABLE 5
Summary of Affinity, Epitope, and cGMP data for
WW01, WW02, WW03, WW04, and WW06 in FabCys Format

| | Characterization in monovalent FabCys format | | | | | | |
|---|---|---|---|---|---|---|---|
| | | | | | Epitope | | |
| | | | | | Bin on | cGMP assay cGMP conc | |
| | Affinity FabCys K_{D} [nM] | | | | hNPR1 | [nM] at 2 μM FabCys | |
| | | hNPR1 + | | rNPR1 + | (-ANP- | Without | +0.075 |
| Antibody | hNPR1 | ANP | rNPR1 | ANP | complex) | ANP | nM ANP |
| WW01 | — | 1.5 | — | 2.0 | B | 30 | 121 |
| WW02 | weak | weak | weak | weak | n.a. | 4 | 20 |
| WW03 | 1000 | 0.1 | 2600 | 1.0 | B | 32 | 108 |
| WW04 | 0.8 | 0.4 | 66 | 73 | A | 4 | 19 |
| WW06 | 5.3 | 12 | weak | weak | A | 281 | 251 |

For candidates WW01 and WW03 no or very weak binding to human and rat NPR1 was observed in the absence of ANP, while the affinities in the presence of ANP were in the low nanomolar to subnanomolar range. Both shared the same epitope bin “B”. The affinity of candidates WW02 was too weak for adequate determination of K_{D} values and the epitope bin. WW04 and WW06 had affinities in the double-digit nanomolar to subnanomolar range, which were independent from the presence or absence of ANP. Both shared the same epitope bin “A”. WW06 was the only candidate which did not exhibit rat cross-reactivity.

While for WW02, WW03 and WW04 no binding to the counter-target hNPR3 in the absence or presence of ANP was observed, additional binding to the counter-target was detected for WW01 as well as for WW06 at higher concentrations.

### Example 5: Reformatting HuCAL® Candidates Into IgG Format

Subcloning from the FabCys vector into an IgG1_LALA vector for expression in mammalian cells was performed via amplification of the Fab-encoding insert using one biotinylated primer and one non-biotinylated primer. The amplified product was bound on streptavidin beads, digested using restriction enzymes, and washed, resulting in the release of the purified insert into the supernatant. The insert was cloned into the acceptor vector, the DNA was transformed and single clones were quality controlled via colony PCR and sequencing.

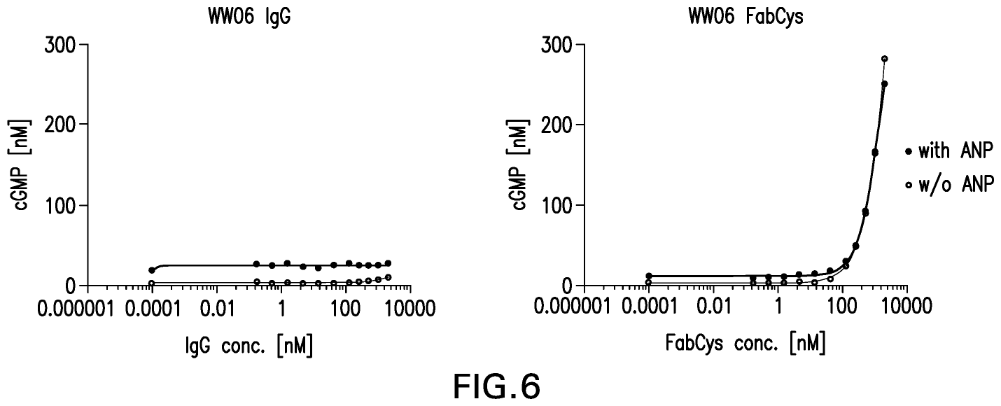

The five functional candidates WW01, WW02, WW03, WW04, and WW06 in IgG format were characterized as described above. Binding data (ELISA, flow cytometry, ANP competition) and functional data (cGMP assay) as well as affinities, and epitope bins are shown in Table 6. Interestingly, WW06 had a significantly increased functional activity in FabCys format compared to IgG format as shown in FIG. 6.

TABLE 6
Summary of Affinity, Epitope, and cGMP data for WW01, WW02, WW03, WW04, and WW06 in IgG Format

| | Characterization in bivalent IgG format | | | | | | | | | | | | |
|---|---|---|---|---|---|---|---|---|---|---|---|---|---|
| | | | | | | | | | Flow | | | | |
| | | | | | | | | | Cytometry | | ANP | | |
| | | | | | | | | | [S/BG] at | | compe- | cGMP assay | |
| | | | | | | | | | 2 μM IgG | | tition | cGMP | |
| | Elisa +/− 100 nM ANP IgG | | | | | | | | | hNPR1 | assay | conc [nM] | |
| | binding/EC₅₀ [nM] | | | | | | | | | CHO | ANP | at 2 μM IgG | |
| | | hNPR1 + | | W74R + | | rNPR1 + | | hNPR3 _+ | hNPR1 | K1 + | compe- | | +0.075 |
| Anti- | | 100 nM | | 100 nM | | 100 nM | | 100 nM | CHO | 100 nM | tition | Without | nM |
| body | hNPR1 | ANP | W74R | ANP | rNPR1 | ANP | hNPR3 | ANP | K1 | ANP | by IgG | ANP | ANP |
| WW01 | no | 1.0 | no | no | no | no | no | no | 19.9 | 222.2 | No | 36 | 197 |
| | specific | | specific | specific | specific | specific | specific | specific | | | | | |
| | binding | | binding | binding | binding | binding | binding | binding | | | | | |
| WW02 | no | 531.6 | no | no | no | no | no | no | 2.0 | 17.4 | No | 6 | 85 |
| | specific | | specific | specific | specific | specific | specific | specific | | | | | |
| | binding | | binding | binding | binding | binding | binding | binding | | | | | |
| WW03 | 42.7 | 1.4 | no | no | no | no | no | no | 2.2 | 214.3 | No | 31 | 192 |
| | | | specific | specific | specific | specific | specific | specific | | | | | |
| | | | binding | binding | binding | binding | binding | binding | | | | | |
| WW04 | 2.5 | 4.2 | 3.5 | 1.8 | no | no | no | no | 1.9 | 46.6 | Yes | 8 | 107 |
| | | | | | specific | specific | specific | specific | | | | | |
| | | | | | binding | binding | binding | binding | | | | | |
| WW06 | 2.5 | 1.1 | 0.9 | 0.8 | no | no | no | no | 4.6 | 1.1 | Yes | 13 | 38 |
| | | | | | specific | specific | specific | specific | | | | | |
| | | | | | binding | binding | binding | binding | | | | | |

### Example 6: Generation of HuCAL® Maturation Libraries

To increase affinity and biological activity of the selected antibody fragments (WW01, WW02, WW03, WW04, and WW06), LCDR3 and HCDR2 regions were exchanged in parallel by diversified cassettes/modules (Prassler et al. (2009): In vitro affinity maturation of HuCAL® antibodies: complementarity determining region exchange and RapMAT technology; Immunotherapy 1 (4), pp. 571-583, the contents of which are hereby incorporated by reference for this purpose), while the framework regions were kept constant. Parental Fab fragments were transferred from the corresponding expression vector into a library cloning vector for affinity maturation.

The generation of HuCAL® maturation libraries was performed for each maturation candidate individually. For LCDR3 optimization, an approximately 400 bp DNA fragment encoding for the LCDR3, framework 4 as well as the constant region of the light chain was removed from the sequence encoding the parental antibody by restriction digest. In order to reduce the background of the parental undiversified sequence the excised fragment was replaced by an approximately 520 bp dummy sequence via ligation, before a repertoire of DNA fragments encoding for diversified LCDR3 regions together with framework 4 and the constant domain (diversified LCDR3 cassette) was inserted via restriction digest and ligation.

In a second library set the HCDR2-encoding sequence was diversified, while the connecting framework regions were kept constant. In order to reduce the background of the parental undiversified sequence an approximately 150 bp DNA fragment containing the parental HCDR2 and the framework 3 sequences was replaced by an approximately 590 bp dummy sequence via restriction digest and ligation, before the diversified HCDR2 cassette (including framework 3) was inserted also via restriction digest and ligation.

The ten maturation libraries were successfully cloned and had library sizes between 9.2×10⁸ and 2.2×10⁹ cfu. Ligation mixtures were electroporated into E. coli cells yielding>10⁸ independent colonies. Amplification of the library was performed as described previously (Rauchenberger et al. (2003): Human combinatorial Fab library yielding specific and functional antibodies against the human fibroblast growth factor receptor 3; J Biol Chem 278 (40), pp. 38194-38205, the contents of which are hereby incorporated by reference for this purpose). For quality control, approx. 10-20 single clones per library were picked randomly and sequenced.

For the selection of affinity improved candidates, phage derived from maturation libraries were subjected to three rounds of maturation panning as described further below. Panning stringency was increased by prolonged washing steps. In addition, off-rate selection was performed (Hawkins et al. (1992): Selection of phage antibodies by binding affinity. Mimicking affinity maturation. In J. Mol. Biol. 226 (3), pp. 889-896, the contents of which are hereby incorporated by reference for this purpose).

### Example 7: Pannings and Screenings—HuCAL®

The maturation libraries were used for four different maturation panning strategies. Strategies #3 and #4 aimed for the enrichment of progenies with improved affinities compared to the parental clones. In addition, strategies #1 and #2 aimed for the enrichment of clones with improved affinities for NPR1 instead of NPR1-ANP-complex. The rationale behind that was the idea to generate candidates which are able to directly active NPR1 by a conformational change. During the panning process, all maturation libraries were kept separately. The panning strategies are summarized in Table 7 in detail. The outputs of the third panning rounds were subsequently sub-cloned into a bacterial expression vector and bacterial lysates (BEL) were used for SET screening.

TABLE 7
Overview of HuCAL ® Maturation Panning Strategies

| | Parental | | | | |
|---|---|---|---|---|---|
| | anti- | | | | |
| Strategy | bodies | 1^{st} round | 2^{nd} round | 3^{rd} round | comments |
| 1 | WW01 | CHO-K1 | hNPR1 | CHO-K1 | Cell/solution |
| | WW02 | NPR1 | solution | NPR1 | panning aiming |
| | WW03 | cells | | cells | for improved |
| | WW04 | | | | candidates |
| | WW06 | | | | |
| 2 | WW01 | CHO-K1 | hNPR1- | CHO-K1 | Cell/solution |
| | WW02 | NPR1 | ANP- | NPR1 | panning aiming |
| | WW03 | cells | complex | cells | for improved |
| | WW04 | | solution | | candidates |
| 3 | WW01 | hNPR1- | CHO-K1 | hNPR1- | Solution/cell |
| | WW02 | ANP- | NPR1- | ANP- | aiming for |
| | WW03 | complex | ANP- | complex | improved |
| | WW04 | solution | complex | solution | NPR1-ANP- |
| | | | Cells | | complex |
| 4 | WW04 | Pread- | Pread- | Pread- | Cell/solution |
| | WW06 | sorp- | sorption | sorption | panning |
| | | tion on | on | on | aiming for |
| | | NPR1- | NPR1- | NPR1- | improved ANP |
| | | ANP | ANP | ANP | competitors |
| | | complex | complex | complex | |
| | | CHO-K1 | hNPR1 | CHO-K1 | |
| | | NPR1 | solution | NPR1 | |
| | | cells | | cells | |

The outputs of the 3rd panning rounds were used for Solution Equilibrium Titration (SET) screening. 88 clones per subcode (2640 clones in total) were analyzed in SET screening for improved affinity for hNPR1 and/or hNPR1-ANP-complex compared to the parental clones.

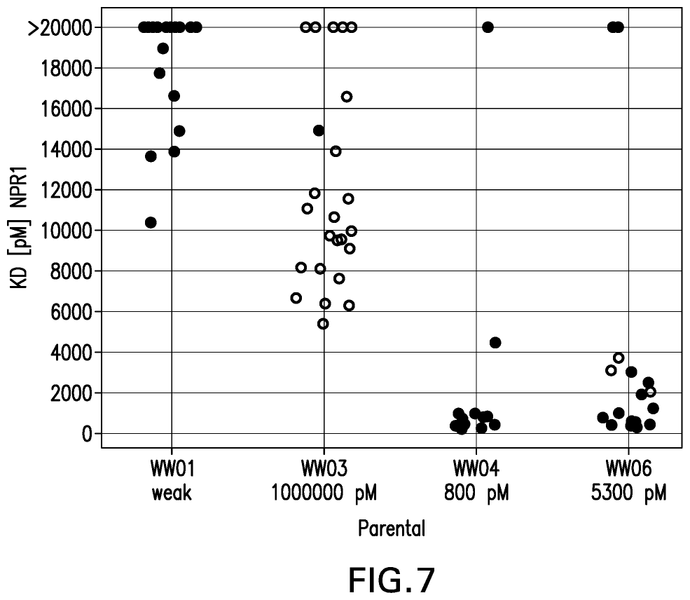

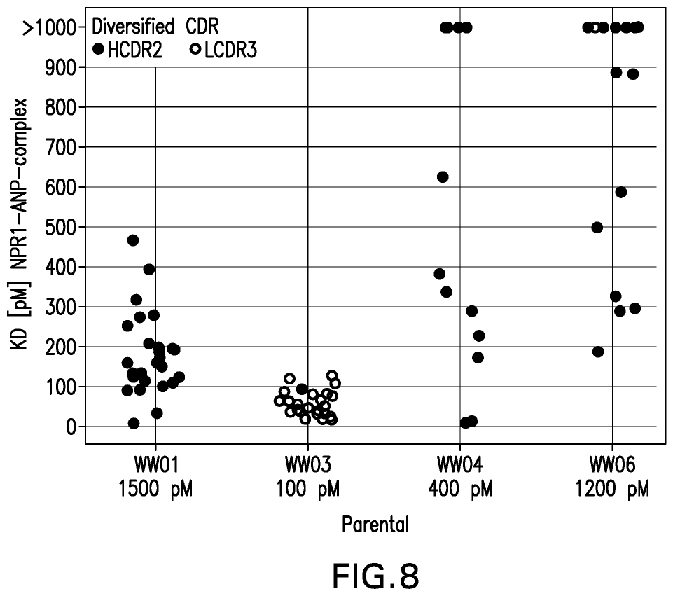

During SET screening, 82 HCDR2 or LCDR3 unique improved derivatives were identified. Compared to their parental clones, the affinities of WW01 and WW03 derivatives were improved up to 20-fold both for hNPR1 and hNPR1-ANP-complex. The affinities of the WW04 derivatives were not improved significantly, while the WW06 derivatives had up to 3-fold improved affinities compared to the parental clone. See FIG. 7 (which shows affinities for hNPR1) and FIG. 8 (which shows affinities for hNPR1-ANP complex). Affinities (K_{D} [pM]) are indicated at the x-axis below the parental clone name for both figures.

### Example 8: Characterization of Matured Candidates—HuCAL®

74 of the 82 improved candidates were successfully subcloned into FabCys format and 61 of the 74 clones passed the production quality control and were characterized in regard to binding to relevant antigens, binding to relevant cell lines, ANP competition, and functional activity in the cGMP production assay in comparison to their parental clones. All 16 derivatives of WW01 and all 27 derivatives of WW03 had up to 20-fold improved binding and functional activity. The majority of the derivatives also showed improved binding and functionality in the presence of ANP. Some derivatives showed binding to W74R (constitutively active hNPR1 mutant), which was not true for the parental FabCys. One of the four derivatives of WW04 had two-fold improved binding and functional activity, while the rest behaved like the parental FabCys. All 14 derivatives of WW06 had improved binding for NPR1 and remained competitive with ANP. The functional activities of 10 of the 14 progenies were improved up to three-fold compared to the parental FabCys. Some derivatives displayed rat cross-reactivity, which was not true for the parental FabCys.

After FabCys characterization, a further 40 of the 61 derivatives were selected for IgG conversion and further characterization. Ten potential candidates shown in Table 8 were then assayed with respect to binding to relevant antigens, binding to relevant cell lines, ANP competition, and functional activity in the cGMP production assay in comparison to their parental clones. They were further analyzed via 3P assay and their affinities for human and rat NPR1 in absence and presence of ANP were determined via SET K_{D} measurement. WW01 and WW03 derivative antibodies were analyzed in IgG format and the WW06 derivatives in FabCys format.

For protein panel profiling (Frese et al. (2013): An automated immunoassay for early specificity profiling of antibodies; mAbs 5 (2), pp. 279-287, the contents of which are herein incorporated by reference for this purpose), 32 different proteins and controls were coated on two 384-well MSD standard plates at a concentration of 1.0 μg/mL at 4° C. overnight. The coating solution was discarded and plates were blocked with 50 μL 3% (w/v) BSA in PBS for one hour at RT on a microtiter plate shaker (˜500 rpm) followed by three washing steps with 50 μL washing buffer (PBS with 0.05% (v/v) Tween 20). Antibody samples were diluted to 100 nM and 10 nM in assay buffer (PBS with 0.5% (w/v) BSA, 0.05% (v/v) Tween 20). As controls, a reference antibody (Fab or IgG, depending on the sample format) and assay buffer were used. Samples and controls were added at 30 μL/well and incubated for three hours at RT on a microtiter plate shaker. The plates were washed three times and 30 μL detection antibody (ECL-labeled anti-human Fab) were added per well and incubated for one hour on a microtiter plate shaker (˜500 rpm). After washing the MSD plate and adding 35 μL/well MSD Read Buffer T with surfactant, electrochemiluminescence signals were detected using a Sector Imager 6000 (Meso Scale Discovery; Gaithersburg, MD, USA). For evaluation, signals of the antibody sample on a certain protein were divided by the respective signals of the reference mAb resulting in a binding ratio (BR). The cumulative binding ratio (CBR) of all proteins except the controls (25 in total) was then calculated: CBR up to 150 represented an antibody or fragment thereof without detectable non-specific binding. Values above represented an antibody or fragment thereof with increased non-specific binding compared to a reference mAb.

TABLE 8
Overview of Matured HuCAL ® Candidates

| Matured | Parental | Matured |
|---|---|---|
| antibody | antibody | CDR |
| XX01 | WW01 | HCDR2 |
| XX02 | WW06 | HCDR2 |
| XX03 | WW03 | HCDR2 |
| XX04 | WW03 | LCDR3 |
| XX05 | WW06 | HCDR2 |
| XX06 | WW03 | LCDR3 |
| XX07 | WW03 | LCDR3 |
| XX08 | WW01 | HCDR2 |
| XX10 | WW06 | HCDR2 |
| XX12 | WW03 | LCDR3 |

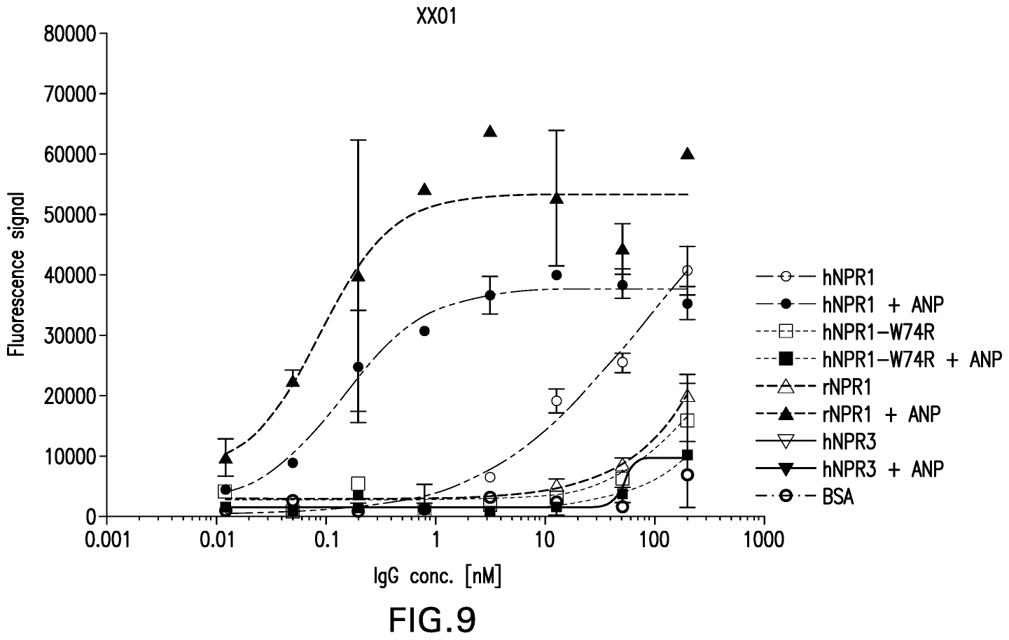

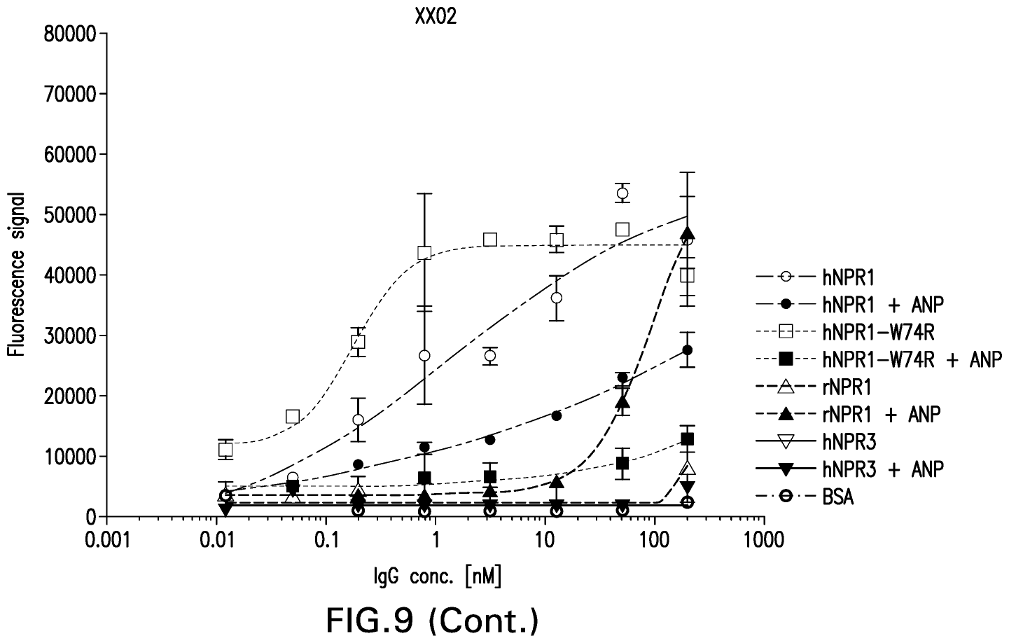

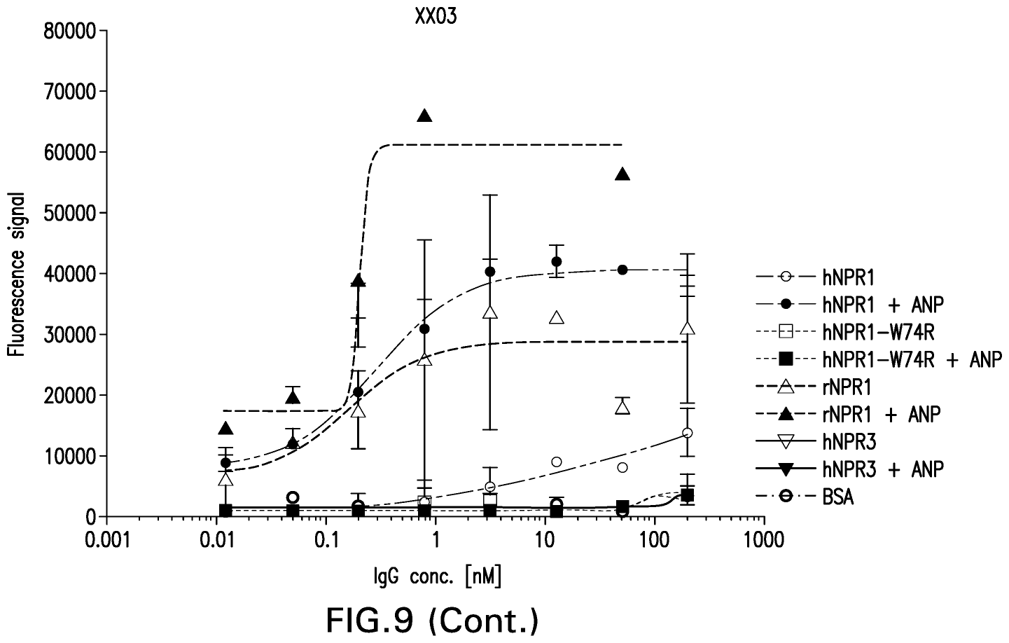

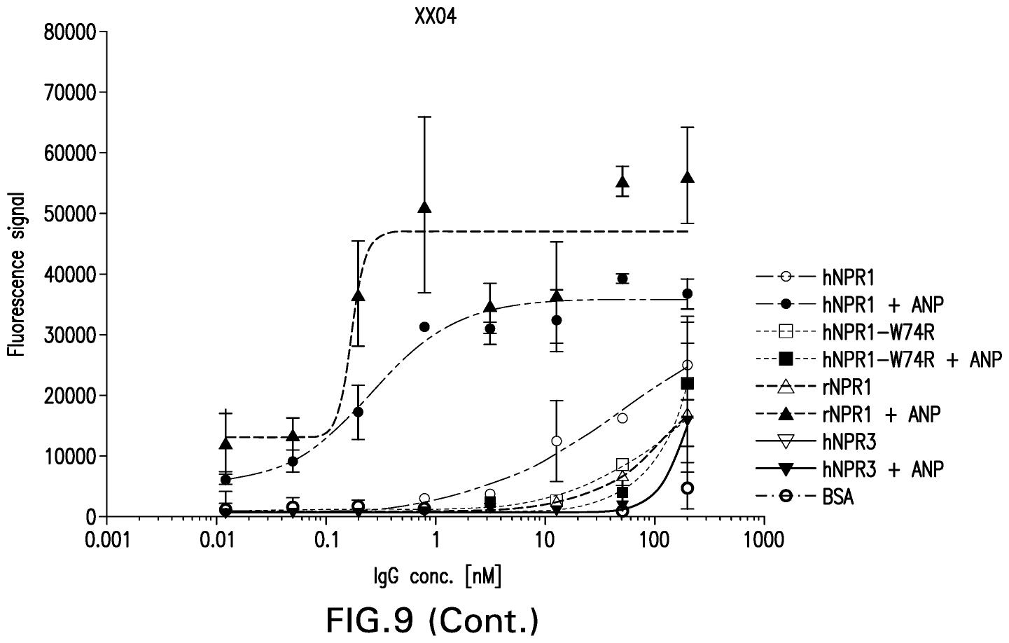

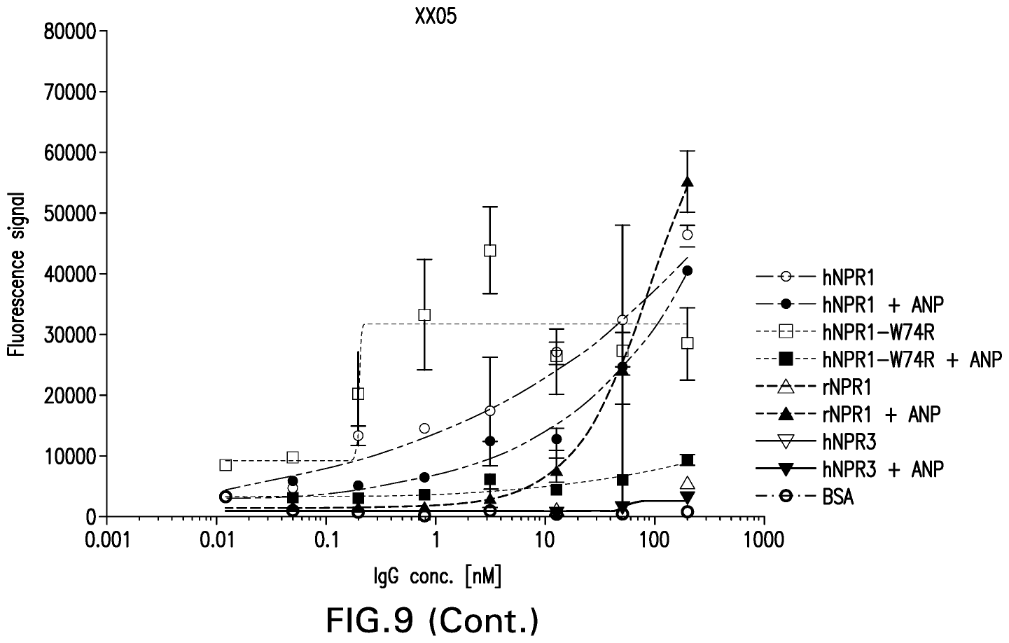

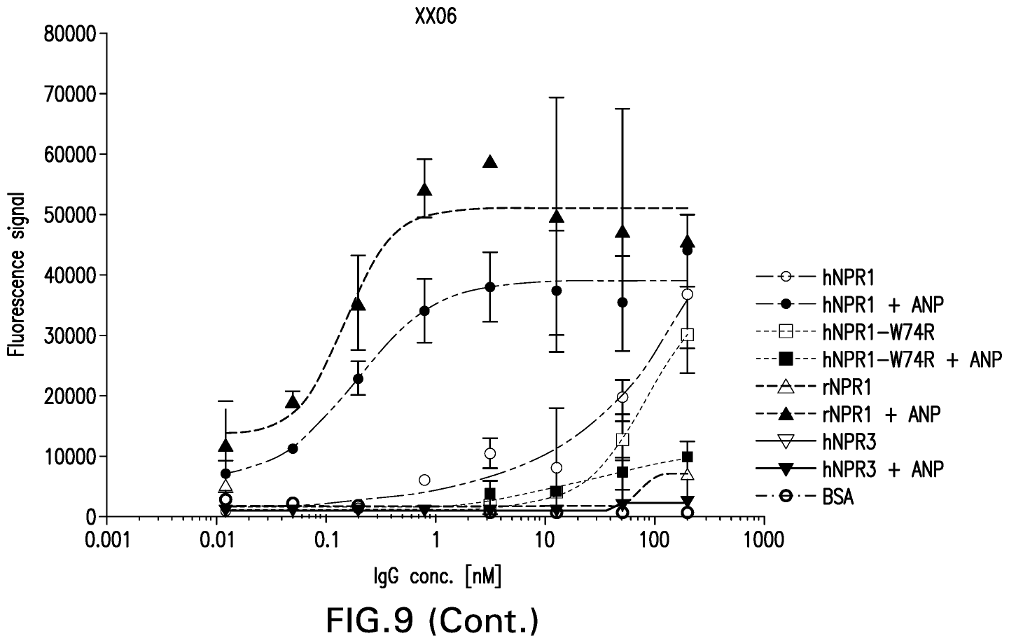

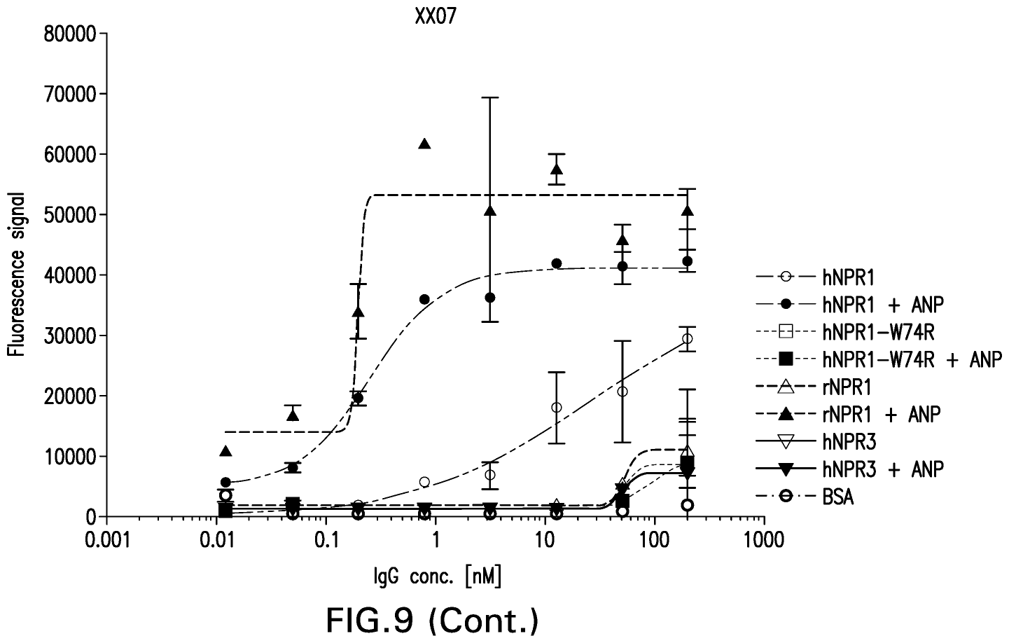

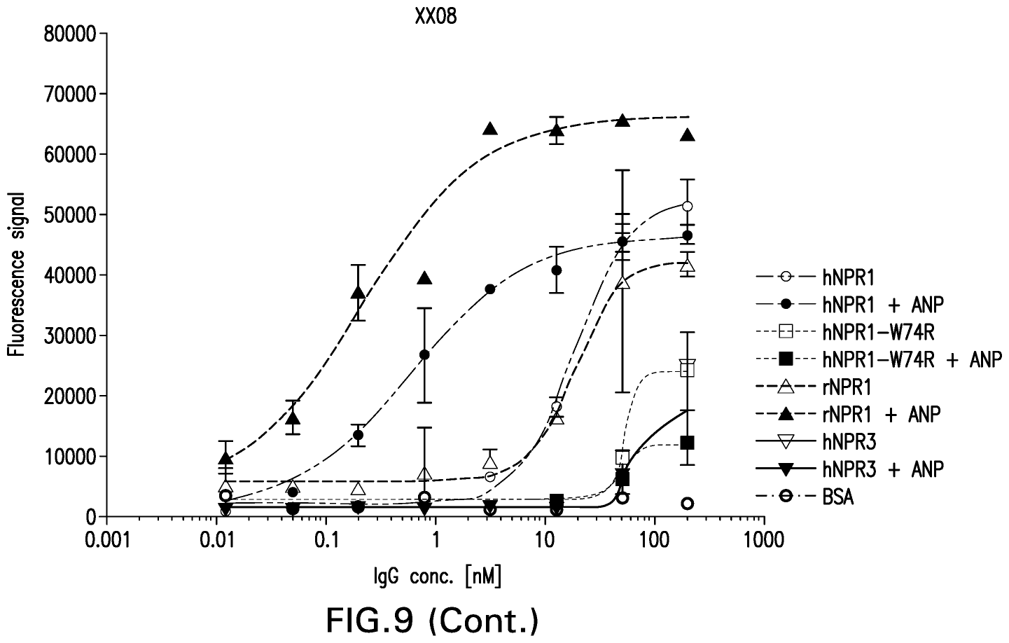

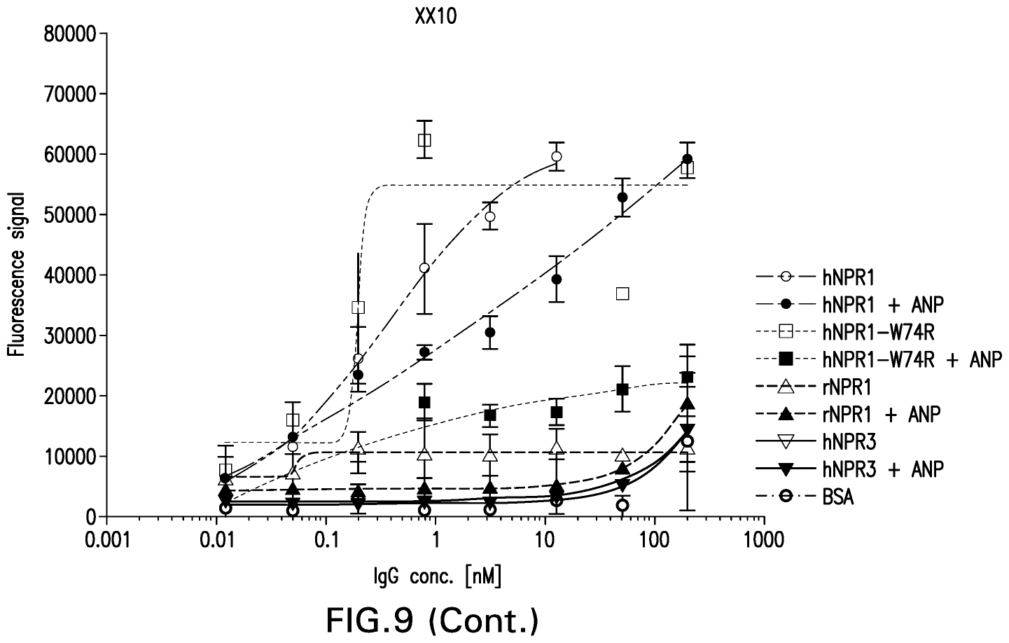

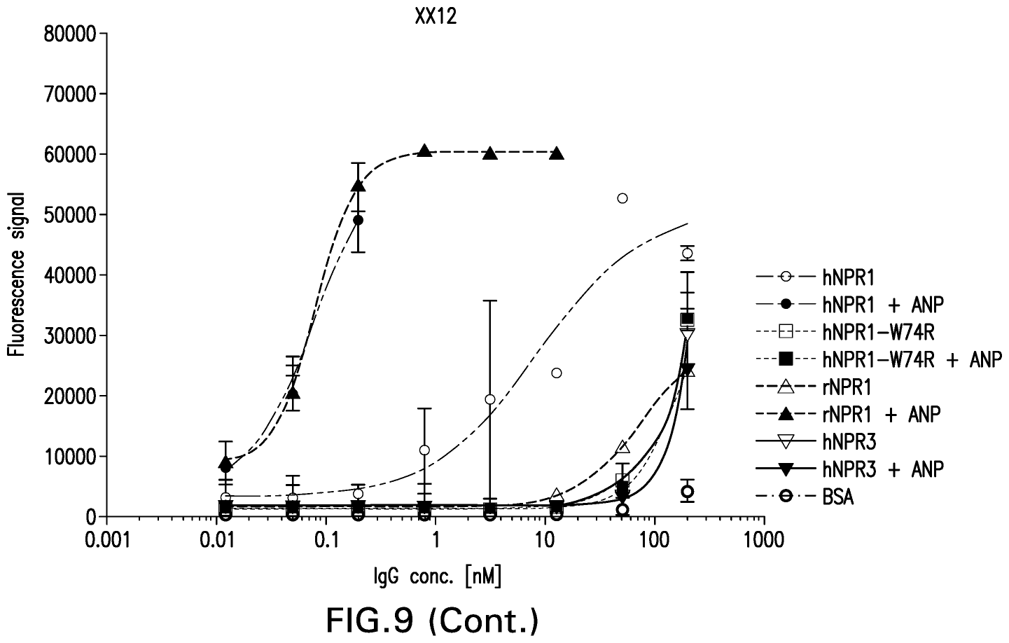

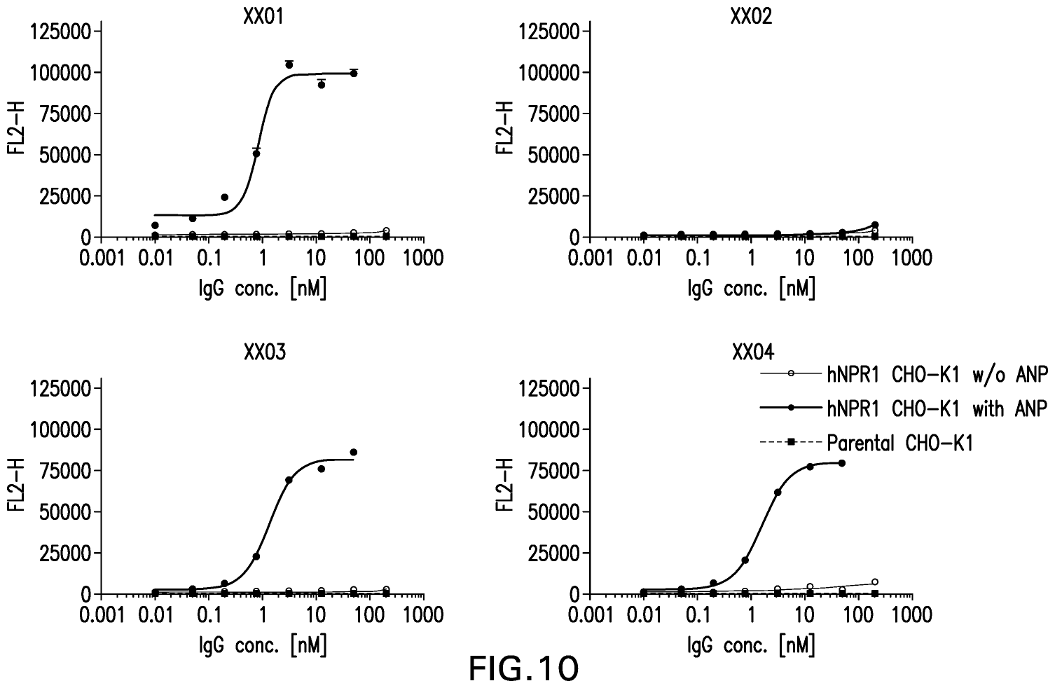

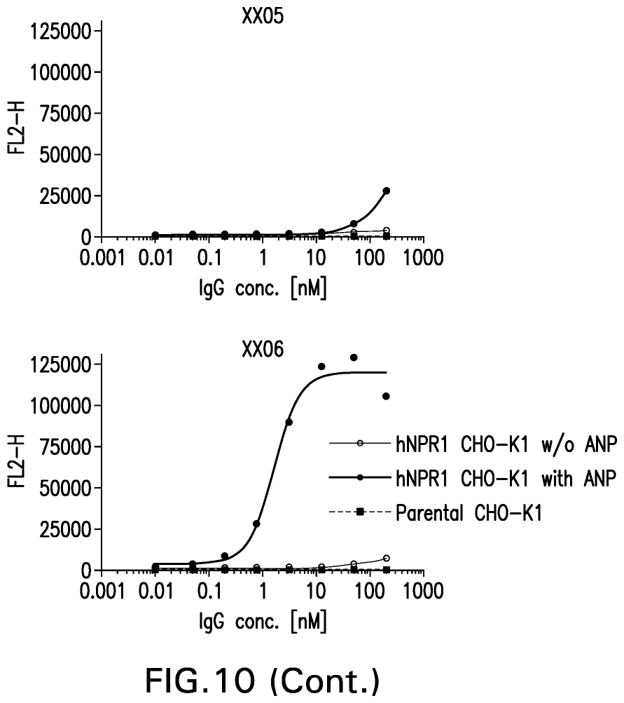

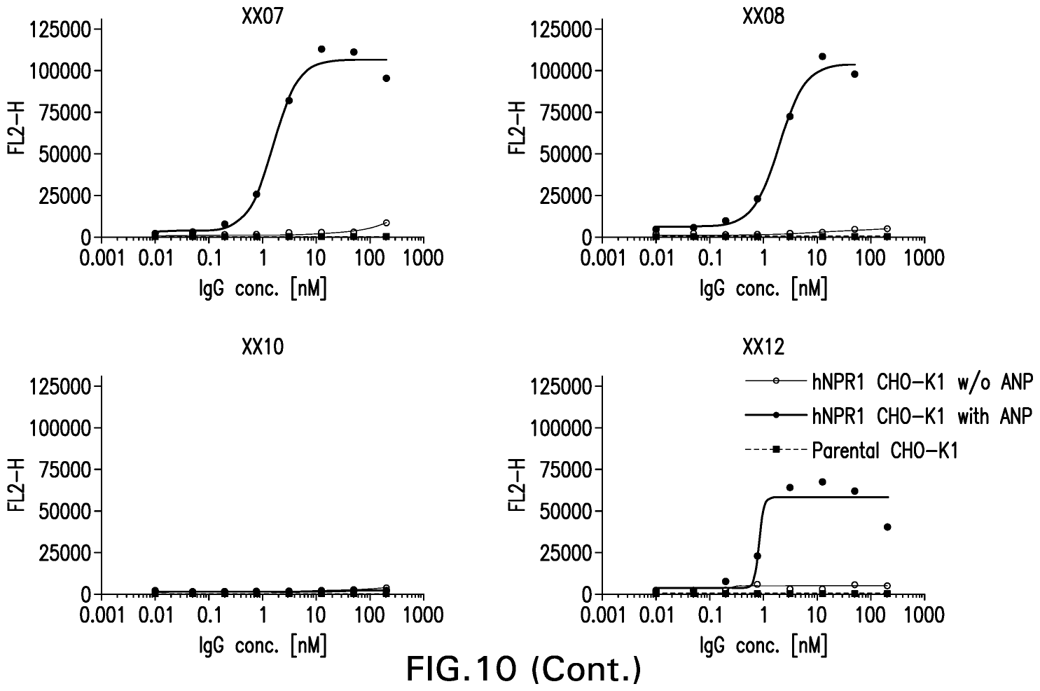

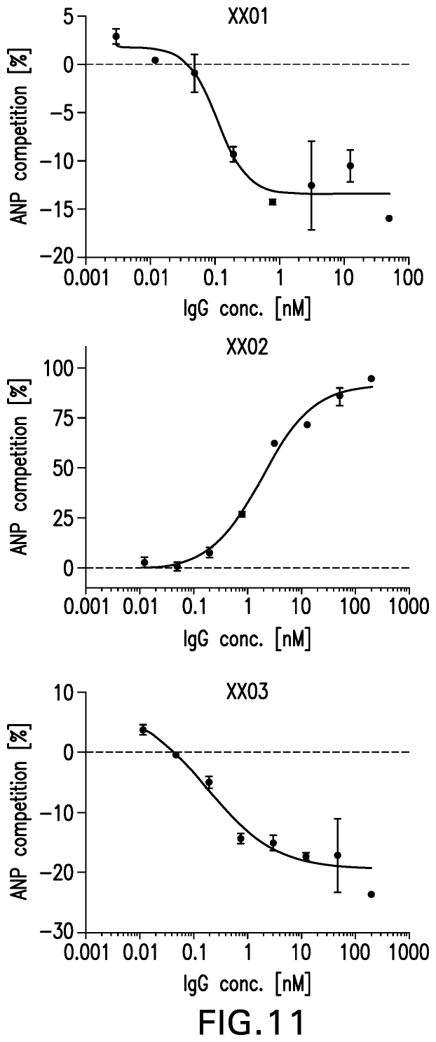

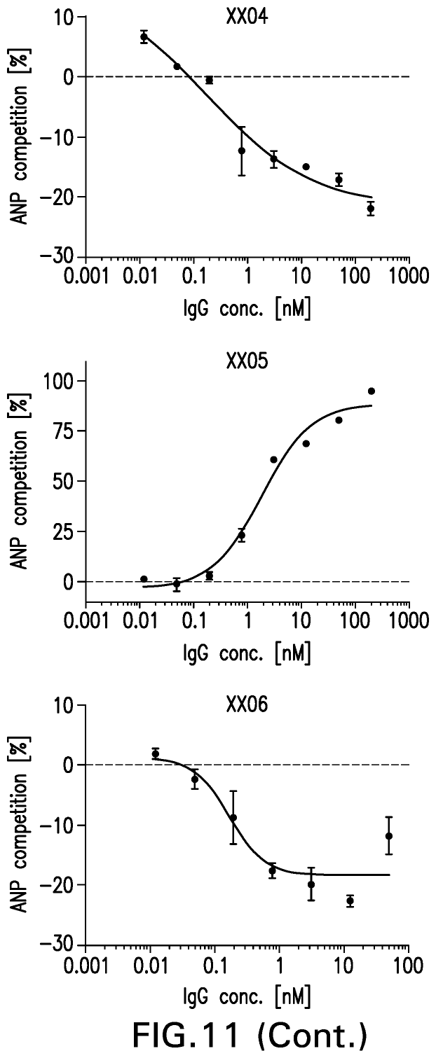

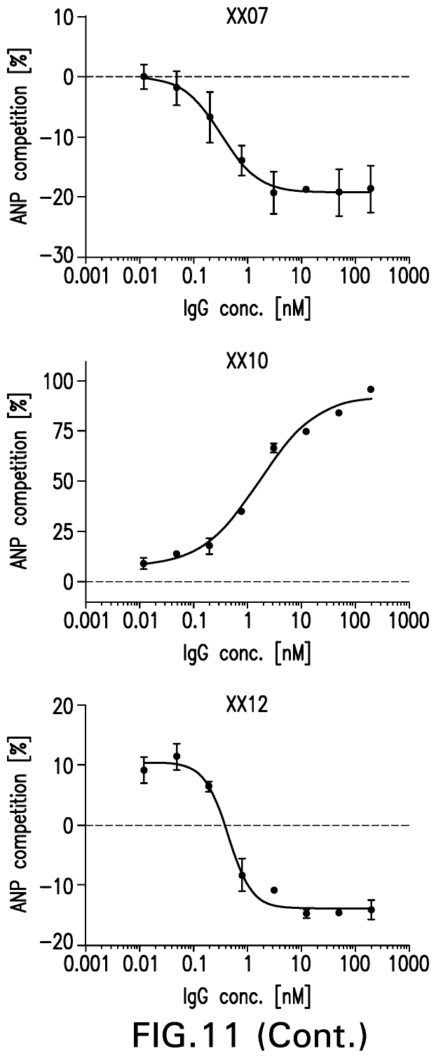

Furthermore, the clones were tested via ELISA for binding to the following antigens: human NPR1, constitutively active human NPR1 mutant (W74R), rat NPR1, human NPR3 (counter target), each in the absence and presence of ANP, and BSA. The clones were also analyzed by flow cytometry for binding to human NPR1 expressing CHO K1 cells in absence and presence of ANP (100 nM) and to parental CHO K1 cells. The binding properties of the 10 candidates are shown in FIGS. 9 and 10. The ANP competition results for nine of the candidates are shown in FIG. 11.

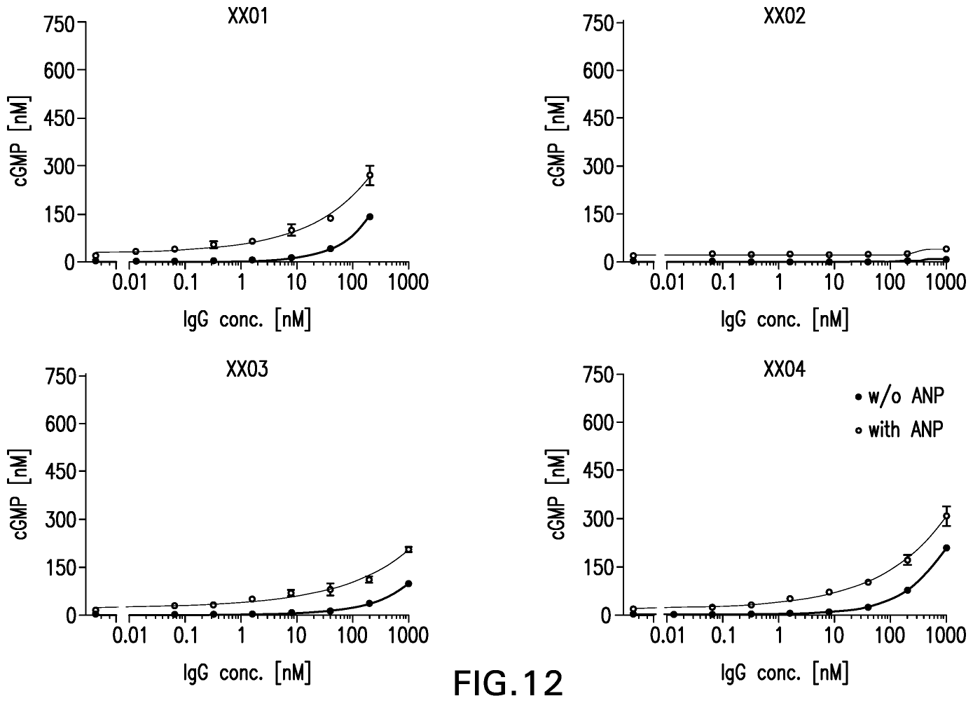

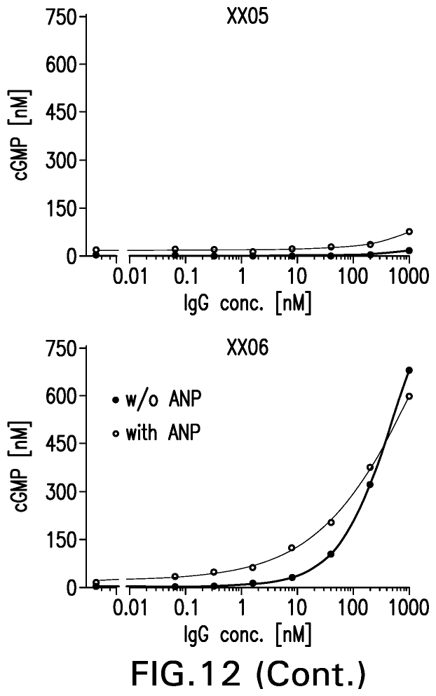

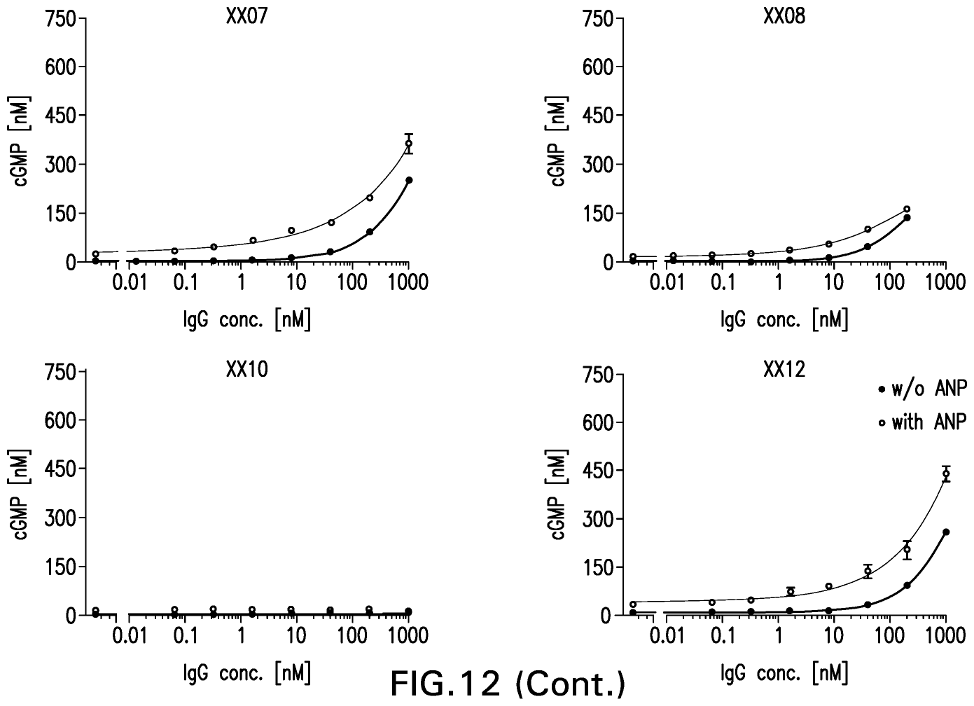

Negative values for XX01, XX03, XX04, XX06, XX07, and XX12 suggest enhancement of ANP binding by these antibodies. The functional activity of the 10 candidates was analyzed using the cellular cGMP production assay and results are shown in FIG. 12. As expected from the functional data of the parental clone WW06, its derivatives XX02, XX05, and XX10 showed weak to no functional activity in the IgG format, whereas they had very high functionality in monovalent FabCys format.

The affinities for XX01-XX08, XX10, and XX12 in monovalent FabCys format were determined via SET K_{D} measurement. The results are summarized in Table 9 in comparison to the affinities of the parental clones (determined in another experiment via Biacore®). The affinities for human and rat NPR1 of WW01 and WW03 derivatives were improved up to 2,300-fold, while the affinities for the NPR1-ANP-complexes were only slightly improved (maximal 5-fold). They had affinities between 10 and 46 nM for hNPR1 and between 100 and 300 pM for hNPR1-ANP-complex. All WW01 and WW03 progenies displayed rat cross-reactivity with rat/human K_{D} ratios <5. The affinities of the WW06 derivatives for human NPR1 and hNPR1-ANP-complex were improved maximally 8-fold and had K_{D} values between 1 and 5 nM, while no binding to rat NPR1 or rat NPR1-ANP-complex could be observed.

TABLE 9
Affinities of Parental and Matured HuCAL ® Candidates

| | Affinity (SET measurement) FabCys K_{D} [nM] | | | |
|---|---|---|---|---|
| Antibody | hNPR1 | hNPR1 + ANP | rNPR1 | rNPR1 + ANP |
| WW01 | weak | 1.5 | weak | 2.0 |
| XX01 | 43 | 0.3 | 1.4 | 0.5 |
| XX08 | 46 | 0.3 | 1.8 | 0.2 |
| WW03 | 1000 | 0.1 | 2600 | 1.0 |
| XX03 | 22 | 0.2 | 1.1 | 0.1 |
| XX04 | 32 | 0.1 | 3.3 | 0.3 |
| XX06 | 10 | 0.1 | 2.3 | 0.3 |
| XX07 | 21 | 0.2 | 2.9 | 0.5 |
| XX12 | 16 | 0.2 | 3.0 | 0.5 |
| WW06 | 5.3 | 12 | — | — |
| XX02 | 1.0 | 1.4 | — | — |
| XX05 | 3.2 | 5.1 | — | — |
| XX10 | 2.2 | 4.0 | — | — |

### Example 9: Cross-Cloning and PTM Removal

Parental clone WW03 had a ‘DG’ site in HCDR2. The majority of the WW03 derivatives (26 out of 27) were diversified in LCDR3. Only one candidate (XX03) was diversified in HCDR2 including the mutation of ‘DG’ into ‘DK’ at amino acid position 54 in the heavy chain variable region (see, e.g., position 54 of SEQ ID NO: 122). The light chains of the functional LCDR3 diversified clones were cross-cloned with the heavy chain of XX03 to engineer these clones without loss of functionality. Furthermore, the ‘DG’ to ‘DK’ mutation was inserted in the original heavy chains of several LCDR3 diversified derivatives. An overview of exemplary cross-cloned and D54K engineered candidates is shown in Table 10.

TABLE 10
Overview of VL-VH Cross-cloned and D54K
Engineered Clones (WW03 derivatives)

| Antibody | Light chain origin | Heavy chain origin |
|---|---|---|
| XX13 | XX06 | WW03 D54K |
| XX14 | XX09 | |
| XX15 | XX04 | XX03 |
| XX16 | XX06 | |
| XX17 | XX07 | |
| XX18 | XX09 | |
| XX19 | XX11 | |
| XX20 | XX12 | |

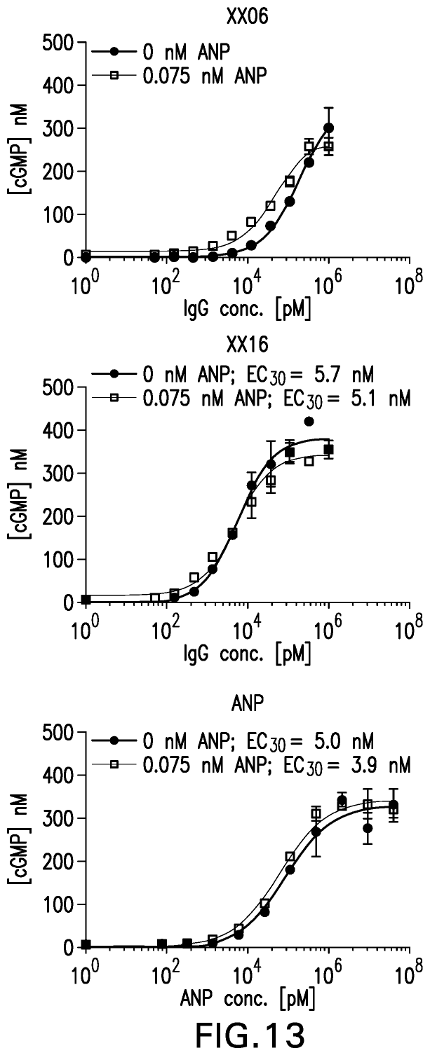

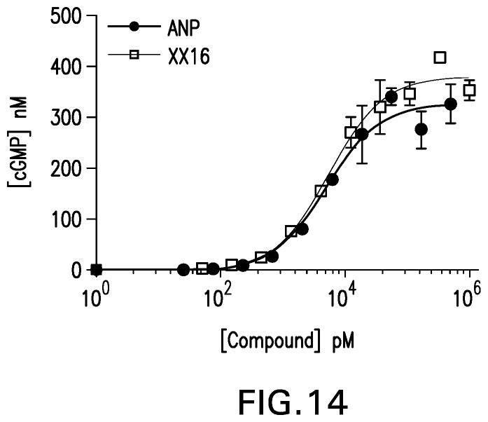

Exemplary functional data of a cross-clone (XX16) compared to the original clone (XX06) and ANP are shown in FIG. 13. All cross-clones had similar or even better functional activity compared to their original clones. XX16 even had a maximal cGMP concentration comparable to the natural ligand ANP in an in vitro study analyzing their activity in hNPR1 transformed CHO cells (see FIG. 14).

Cross-cloned and PTM removed clones were tested for their specificity via 3P assay. Both D54K engineered clones XX13 and XX14 showed non-specific binding to several antigens and were deselected, while no cross-clone showed non-specific binding. Results are shown in Table 11.

TABLE 11
In vitro functional data for XX15 and XX16

| | cGMP Generation for human, rat, cyno NPR1 and human NPR2 expressing CHO cells | | | | | | | |
|---|---|---|---|---|---|---|---|---|
| | | hNPR1 | | rNPR1 | cNPR1 | cNPR1 | | |
| | hNPR1 | Y max | rNPR1 | Y max | EC50 (nM) | Y max | hNPR2 | hNPR2 |
| | EC50 (nM) | (% of ANP) | EC50 (nM) | (% of ANP) | +/− | (% of ANP) | EC50 | Y max |
| Antibody | +/− STDEV | +/− STDEV | +/− STDEV | +/− STDEV | STDEV | +/− STDEV | (nM) | (% of ANP) |
| XX16 | 3.2 +/− 0.4 | 97 +/− 1 | 8 +/− 2 | 94 +/− 8 | 12 +/− 2 | 86 +/− 2 | >500 | <1 |
| XX15 | 9.4 +/− 0.4 | 98 +/− 7 | 23 +/− 6 | 100 +/− 10 | 30 +/− 2 | 95 +/− 3 | >500 | <1 |
| XX18 | 40 +/− 10 | 110 +/− 10 | 26 +/− 9 | 99 +/− 8 | 50 +/− 10 | 90 +/− 5 | >500 | <1 |

### Example 10: Crystal Structure of Anti-NPR1 Antibodies

Crystal structures for several molecules in complex with hNPR1 were created as described below.

For Fab03-WW03, the Fab construct of WW03 was complexed to the extracellular domain of hNPR1 (C264T) with a molar ratio of 2 Fab molecules for every 1 NPR1 molecule. The complex was incubated for 1 hour in the cold room rocking and then loaded onto a Superdex 200 16/60 column in the buffer 20 mM HEPES pH7.4, 100 mM NaCl. The complexed protein was separated from a small aggregate peak and the excess Fab and then concentrated to 19.8 mg/mL. The complex crystallized in space group P212121 and diffracted to a resolution of 2.89 Å. The model was built using molecular replacement with the hNPR1 structure and a Fab molecule, iteratively built in Coot and refined with Buster to an Rfree of 21.7%.

For Fab06-WW06, the Fab construct of WW06 was complexed to the extracellular domain of hNPR1 (C264T) with a molar ratio of 2 Fab molecules for every 1 NPR1 molecule. The complex was incubated for 1 hour in the cold room rocking and then loaded onto a Superdex 200 16/60 column in the buffer 20 mM HEPES pH7.4, 100 mM NaCl. The complexed protein was separated from a small aggregate peak and the excess Fab and then concentrated to approximately 20.0 mg/mL. The complex crystallized in space group P212121 and diffracted to a resolution of 2.17 Å. The model was built using molecular replacement with the hNPR1 structure and a Fab molecule, iteratively built in Coot and refined with Buster to an Rfree of 20.9%.

For Fab16-XX16, the Fab construct of XX16 was complexed to the extracellular domain of hNPR1 (C264T) with a molar ratio of 2 Fab molecules for every 1 NPR1 molecule. The complex was incubated for 1 hour in the cold room rocking and then loaded onto a Superdex 200 16/60 column in the buffer 20 mM HEPES pH7.4, 100 mM NaCl. The complexed protein was separated from a small aggregate peak and the excess Fab and then concentrated to approximately 20.0 mg/mL. The complex crystallized in space group P212121 and diffracted to a resolution of 3.02 Å. The model was built using molecular replacement with the hNPR1 structure and a Fab molecule, iteratively built in Coot and refined with Buster to an Rfree of 24.4%.

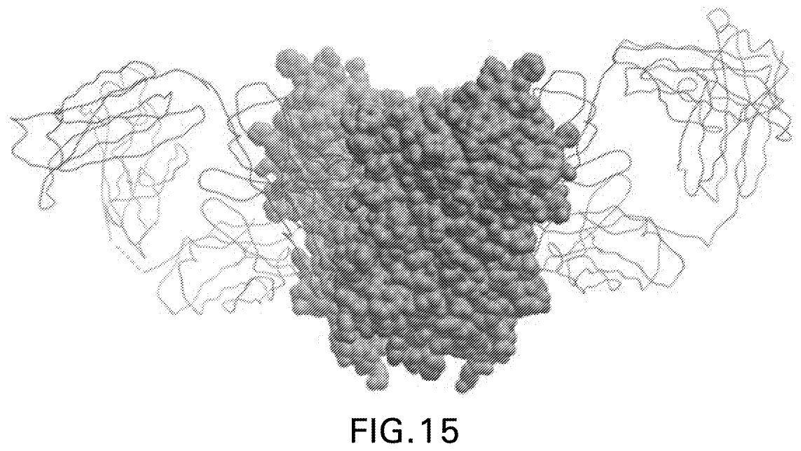

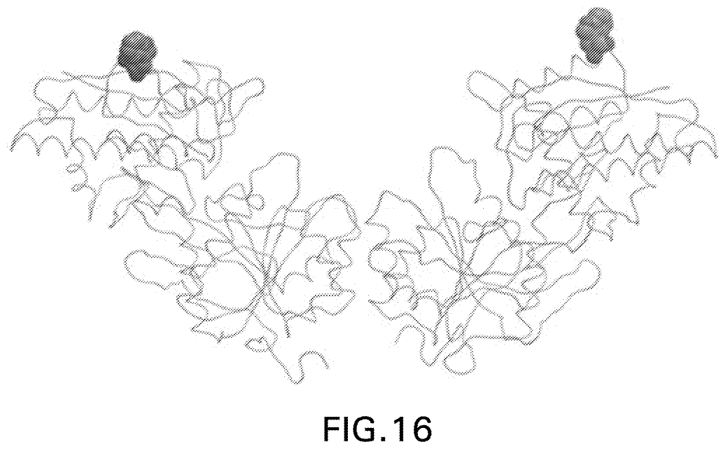

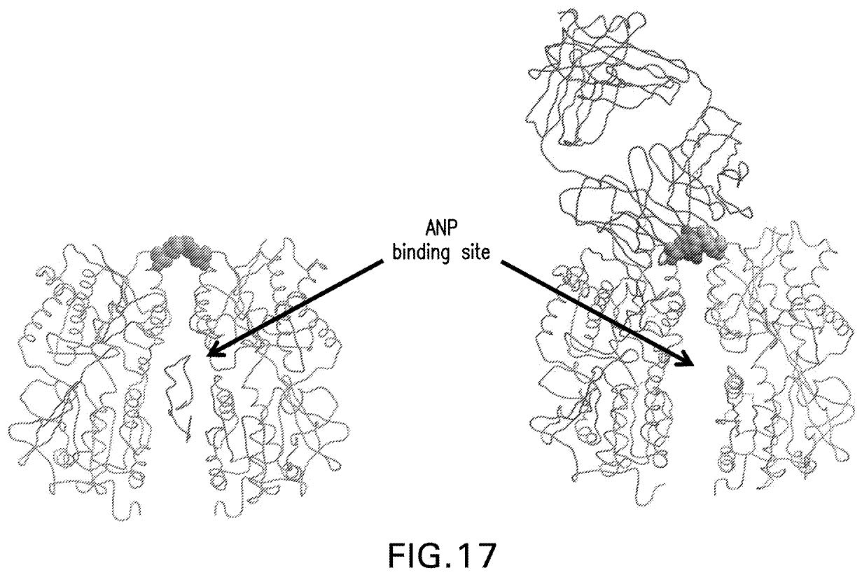

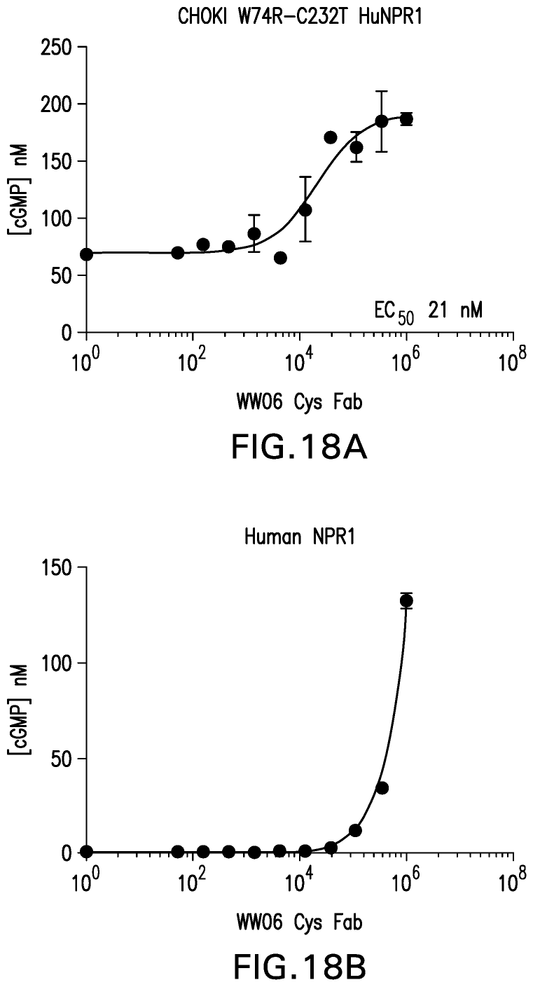

The crystal structure of Fab06 in complex with hNPR1 is shown in FIG. 15. FIG. 16 shows the conformation of the hNPR1 ECD as it would be in complex with Fab06; the Fab06 were removed from the image to more clearly reveal the conformation of hNPR1 induced by Fab binding. The structures shown in FIGS. 15 and 16 explain the discrepancy between Fab and IgG functional data shown in FIG. 6. The Fab C-termini are 180 degrees apart precluding a single IgG spanning a receptor dimer. The structure of hNPR1-ECD in complex with ANP (which was also determined as a part of this work) is shown for comparison in FIG. 17 (left), and the crystal structure of Fab16 (the XX16 antibody in Fab format) in complex with hNPR1 extracellular domain (right). The WW06 Fab was significantly more potent (cGMP production) when tested on CHO cells expressing hNPR1 W74R/C232T (constitutively active mutant) compared to WT hNPR1 (FIG. 18, panels A and B) suggesting that the mutation destabilizes the head-to-head conformation of NPR1 and facilitates antibody binding/receptor activation.

### Example 11: Ylanthia® Phage Library Panning and Screening

Six panning strategies were performed, which reflected the most successful strategies from the initial pannings (HuCAL®) or modifications of these strategies aiming for specific methods of action. The panning strategies are summarized in Table 12 in detail. Strategies #2 and #5 were identical to strategies performed in the initial HuCAL® campaign and were selected because all five initial functional candidates were derived from these panning strategies. Strategies #3 and #4 were variations from initial strategies with focus on hNPR1 alternating with hNPR1 expressing cells. In addition, a constitutively active mutant of NPR1 (W74R) was used as an antigen in strategies #1 and #6. Bacterial lysates (BEL) of the outputs of the 3rd panning rounds in phage display vector pYPDis were directly used for primary and secondary screenings.

TABLE 12
Overview of panning strategies - Ylanthia ®

| Strategy | 1^{st} round | 2^{nd} round | 3^{rd} round | Comments |
|---|---|---|---|---|
| 1 | W74R hNPR1 capture | W74R hNPR1 capture | W74R hNPR1 capture | Fc capture panning with active |
| | | | | W74R mutant only |
| 2 | hNPR1-ANP-complex | rNPR1-ANP-complex | hNPR1-ANP-complex | Solution/Fc capture panning |
| | solution | capture | solution | aiming for enrichment of NPR1- |
| | | | | ANP-complex |
| 3 | CHO-K1 NPR-ANP- | hNPR1-ANP-complex | CHO-KI NPR-ANP- | Cell/solution panning aiming for |
| | complex cell | solution | complex cell | enrichment of NPR1-ANP-complex |
| | | | | stabilizers |
| 4 | hNPR1 solution | CHO-KI NPRI cell + | hNPR1 solution + | Solution/cell aiming for |
| | | ANP elution | ANP elution | enrichment of ANP competitors |
| 5 | pre-adsorption on | pre-adsorption on | pre-adsorption on | Solution panning aiming for |
| | NPR1-ANP complex, | NPR1-ANP complex, | NPR1-ANP complex, | enrichment of ANP competitors |
| | save the unbound | save the unbound | save the unbound | |
| | phage still in solution, | phage still in solution, | phage still in solution, | |
| | bind to | bind to | bind to | |
| | hNPR1 in solution | hNPR1 in solution | hNPR1 in solution | |
| | without ANP and | without ANP and | without ANP and | |
| | capture NPR1/phage | capture NPR1/phage | capture NPR1/phage | |
| | complexes | complexes | complexes | |
| 6 | CHO-K1 NPR1 cell | W74R hNPR1 capture | CHO-K1 NPR1 cell | Cell/Fc capture panning with |
| | | | | human antigen and active W74R |
| | | | | mutant |

The outputs of the 3^{rd} panning rounds were analyzed for binding to relevant antigens and cell lines. 368 clones per subcode (in total 4416 clones) were screened in ELISA-based primary screening on human NPR1 in absence and presence of ANP, constitutively active hNPR1 mutant (W74R) and counter-target human hNPR3. The primary screening yielded 810 hits, which were analyzed with respect to binding of relevant cell lines (human NPR1 expressing CHO-K1 cells in absence and presence of ANP, parental CHO-K1 cells) and rat NPR1 in secondary screening. In total, 380 clones from primary and secondary screening were selected for sequencing with priority for exclusive binding to NPR1-ANP-complex, good cell binding, and rat cross-reactivity. The VL and VH sequencing resulted in 138 HCDR3 unique clones with different binding properties (Table 13). Of these clones six bound only in presence of ANP.

TABLE 13
Binding properties of HCDR3 unique hits - Ylanthia ®

| | | | Binding to: | | |
|---|---|---|---|---|---|
| | | | | | hNPR1 |
| Number of unique candidates | | | hNPR1 | rNPR1 | expressing cells |
| 140 (of which 6 | 53 human/rat | 2 human/rat cross-reactive cell binders | Yes | Yes | Yes |
| clones bound only | cross-reactive | 51 human/rat cross-reactive | Yes | Yes | No |
| in presence of | | (not binding to cells) | | | |
| ANP) | 79 human | 14 human specific cell binders | Yes | No | Yes |
| | specific | 65 human specific | Yes | No | No |
| | | (not binding to cells) | | | |

Since candidate WW06 derived from the initial HuCAL® pannings was significantly PGP-more active in FabCys format compared to IgG format, the functional screening was performed in FabCys format rather than IgG format.

The sub-cloning of 138 HCDR3 unique clones into the FabCys format was performed via YClone®. 111 out of 138 clones were successfully converted into FabCys format and 95 clones were selected for further analysis. 92 of the 95 FabCys passed the production quality control and were analyzed in detail. Afterwards, 30 of the 92 clones with the most promising properties were selected for IgG conversion via AmplyFly®, exploratory scale expression and S-DAS. 24 of the 30 IgGs passed the production quality control and were analyzed in detail.

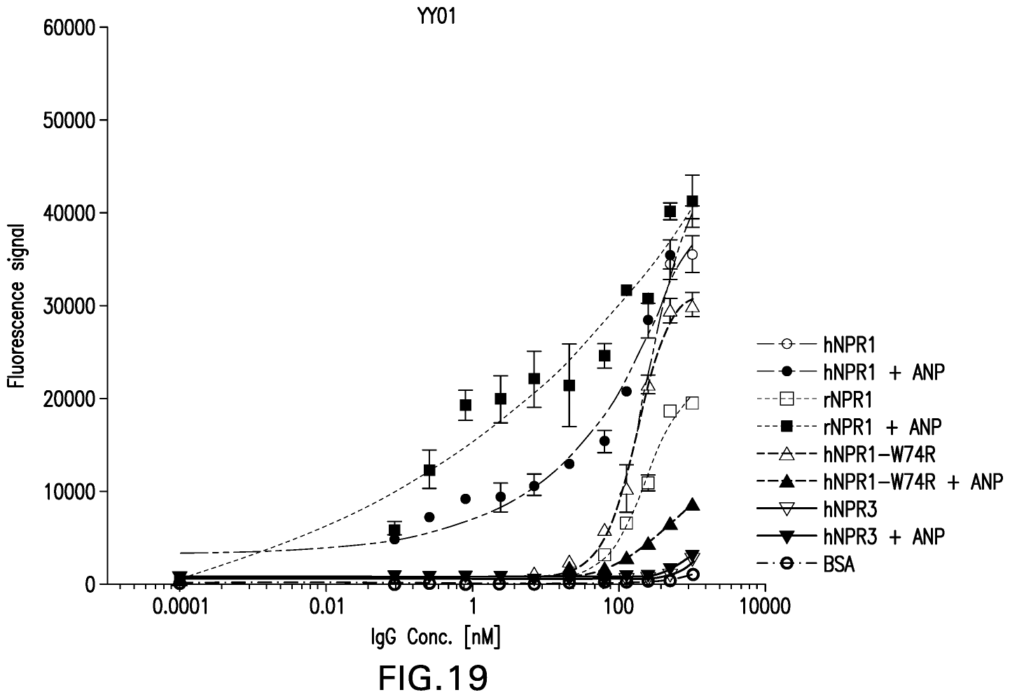

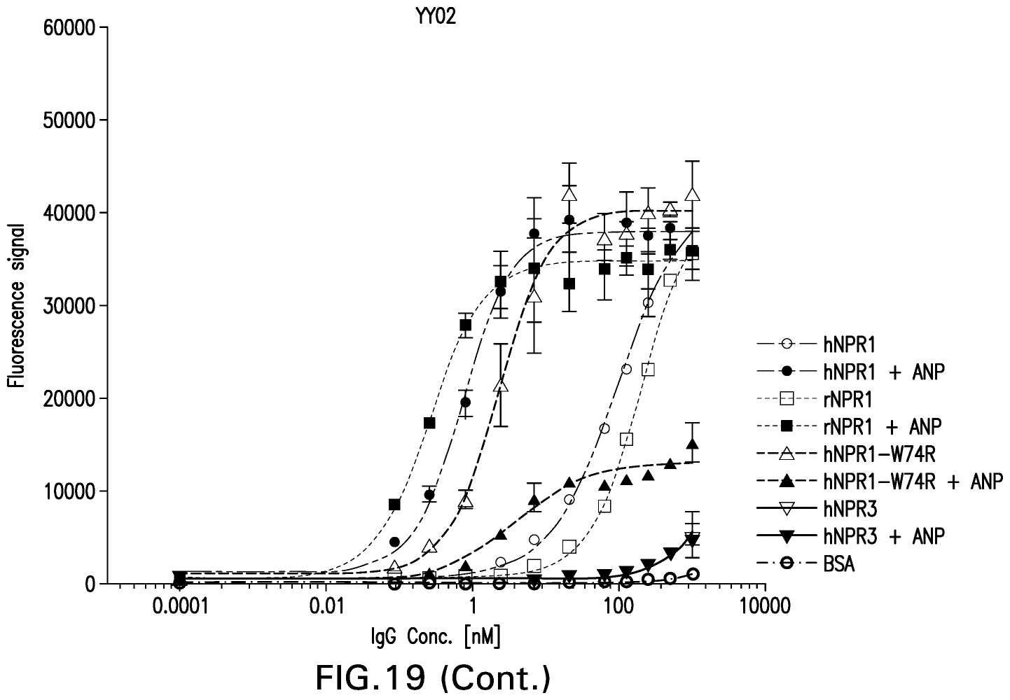

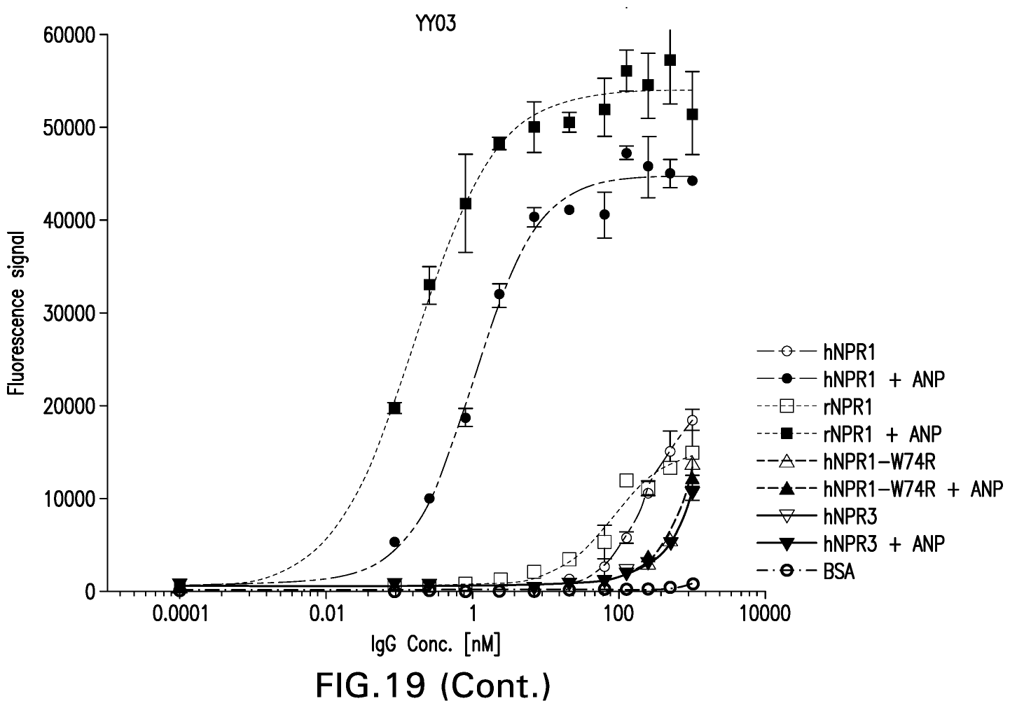

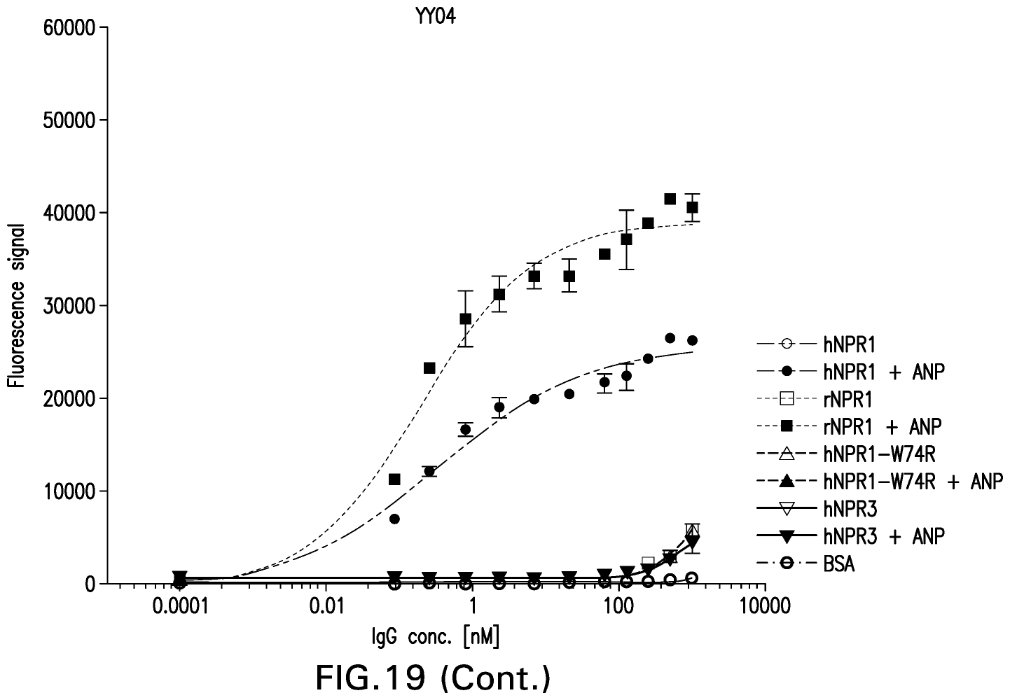

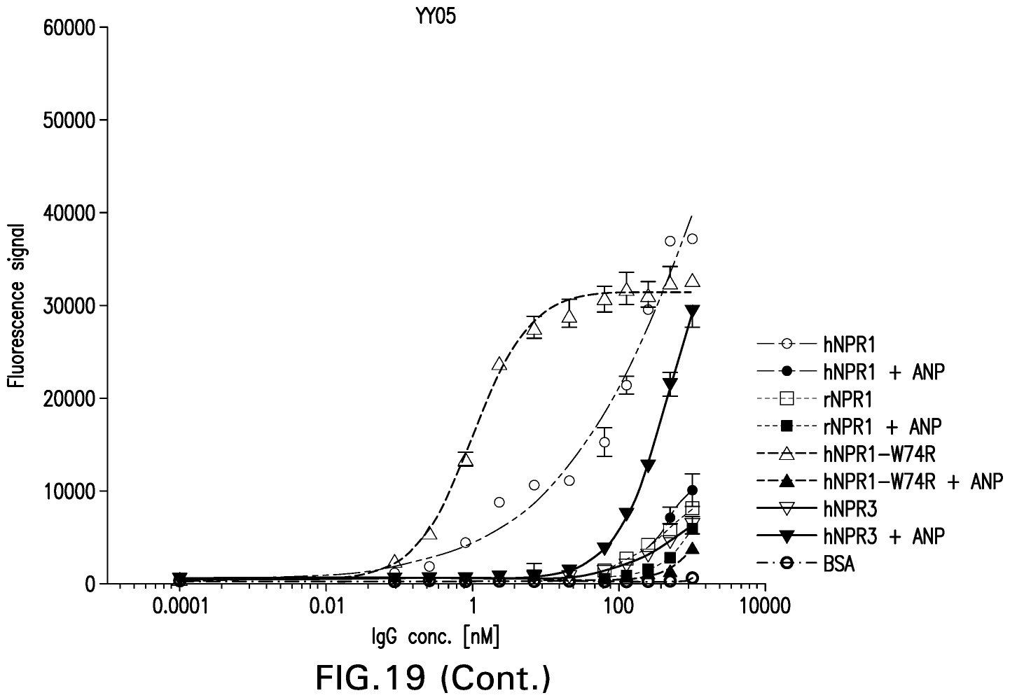

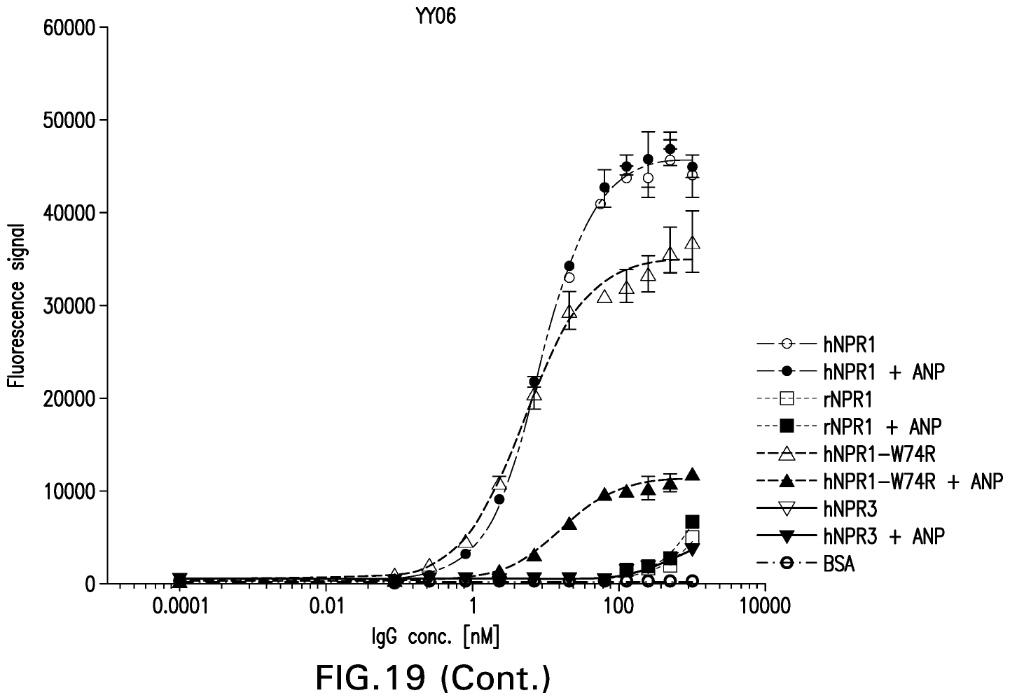

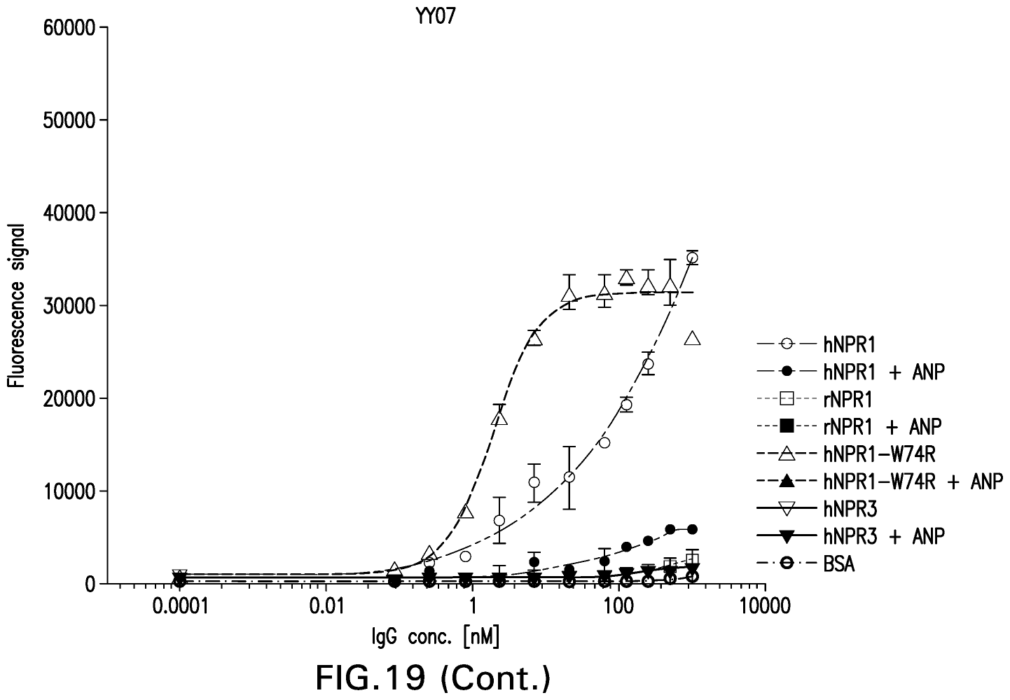

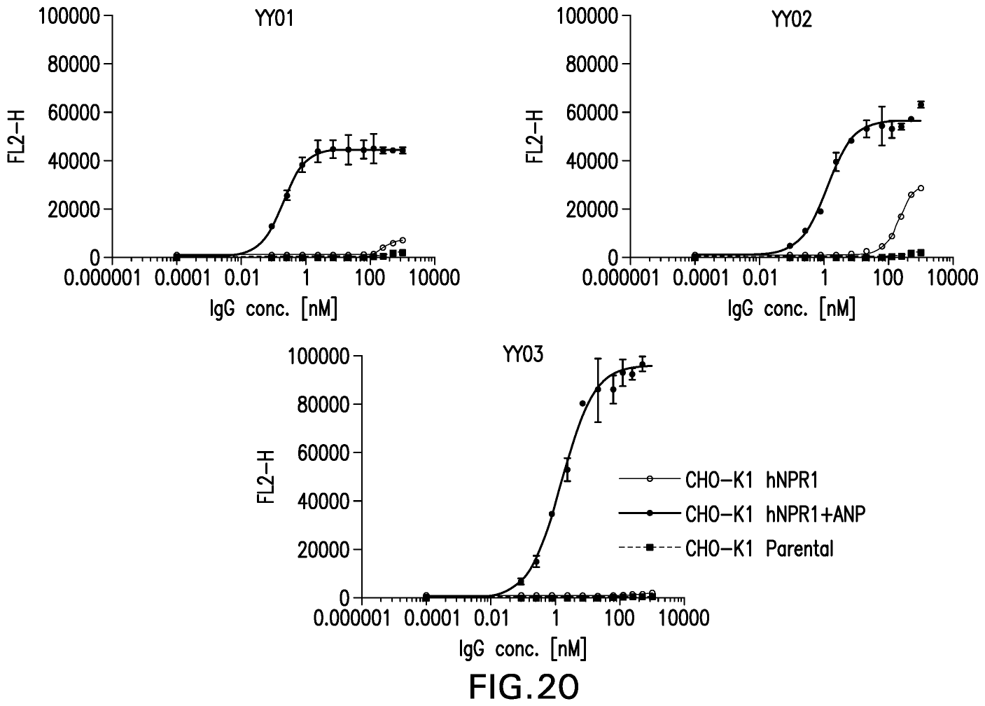

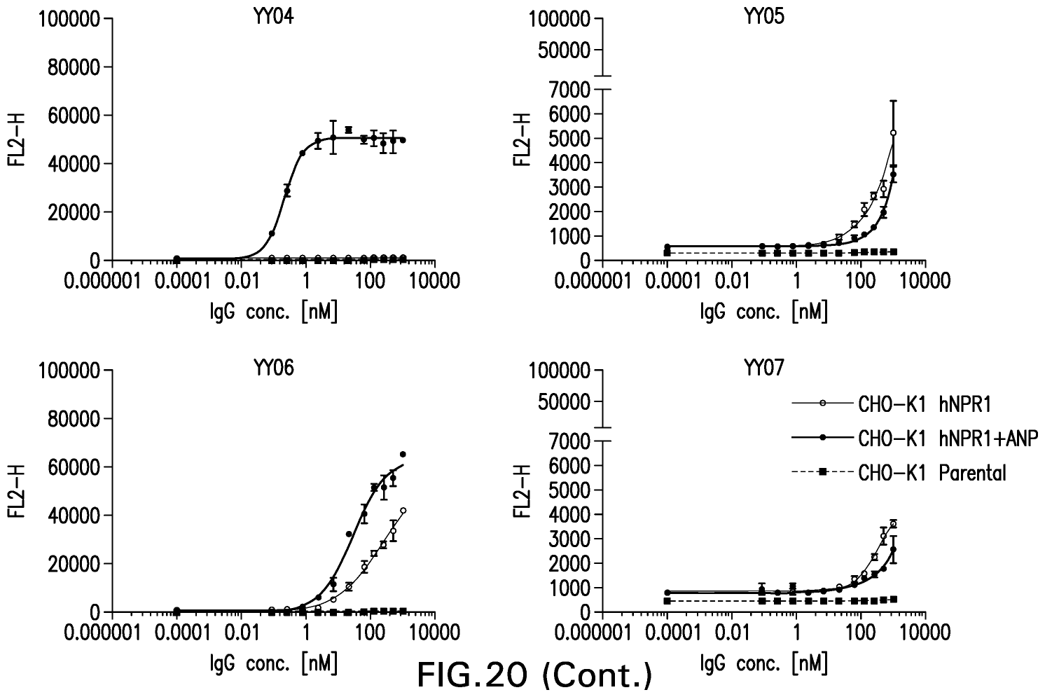

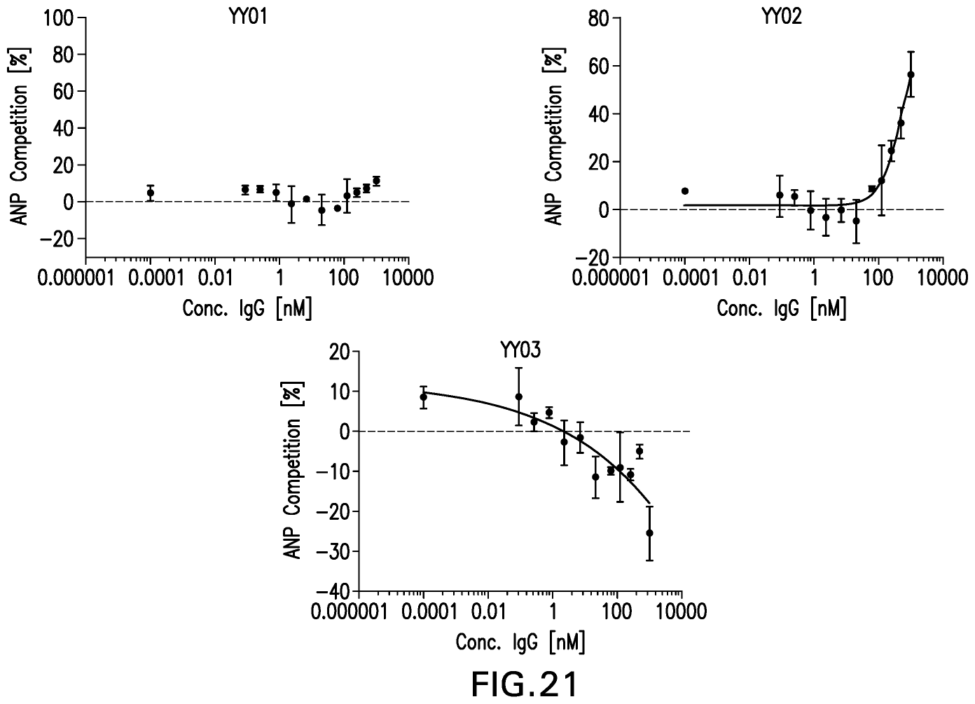

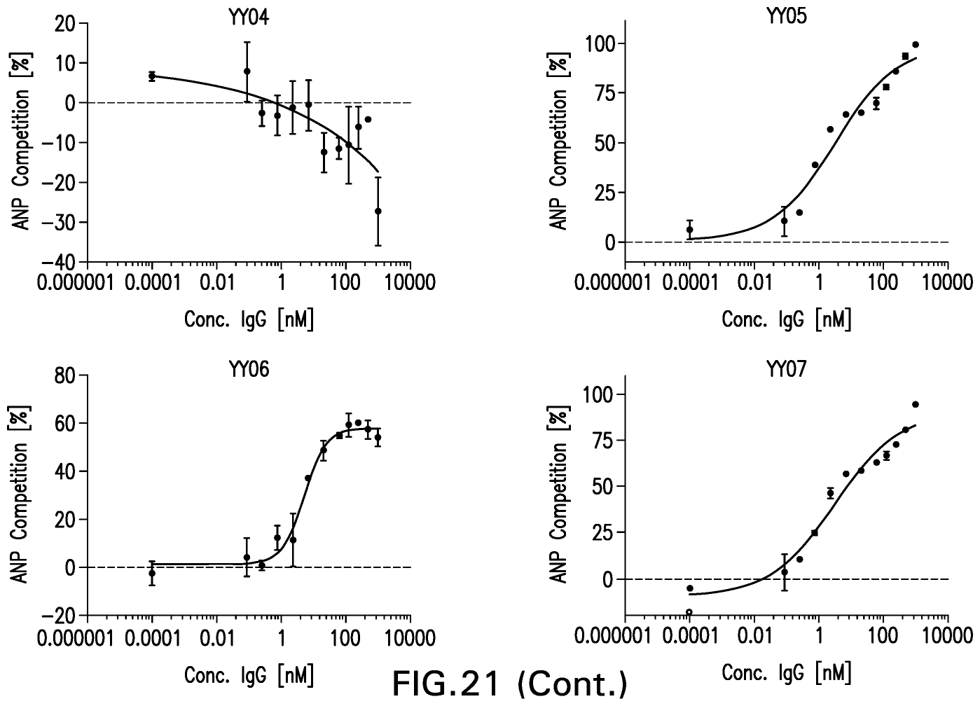

All 92 FabCys and 24 IgGs were tested for binding to relevant antigens via ELISA and relevant cell lines by flow cytometry. Furthermore, the clones were tested for ANP competition and functional activity in the cellular cGMP production assay. Eight functional candidates were identified and analyzed for specificity in the Protein Panel Profiling assay (3P assay) in IgG format. For comparison, one of the functional candidates from the initial campaign (WW03) was analyzed. YY02 and YY03 showed low non-specific binding; and YY01, YY04, YY05, YY06, YY07 and WW03 did not show non-specific binding in this assay. All 92 FabCys and 24 IgGs were tested via ELISA for binding on the following antigens: human NPR1, constitutively active human NPR1 mutant (W74R), rat NPR1, human NPR3 (counter target) in the absence or presence of ANP (100 nM) and irrelevant antigens. The clones were also tested by flow cytometry for binding on human NPR1 expressing CHO K1 cells in the absence and presence of ANP and on parental CHO K1 cells. The binding properties of the seven functional candidates in IgG format are shown in FIGS. 19 and 20. All 92 FabCys and 24 IgGs were tested for ANP competition. 22 out of 92 FabCys and 12 out of 24 IgGs showed a significant ANP competition >70% at a concentration of 1 μM FabCys/IgG. The results of the seven functional candidates in IgG format are shown in FIG. 21. YY02, YY05, YY06, and YY07 showed clear ANP competition. YY03 and YY04 showed a “negative” ANP competition in this assay indicating the stabilization of the NPR1-ANP-complex.

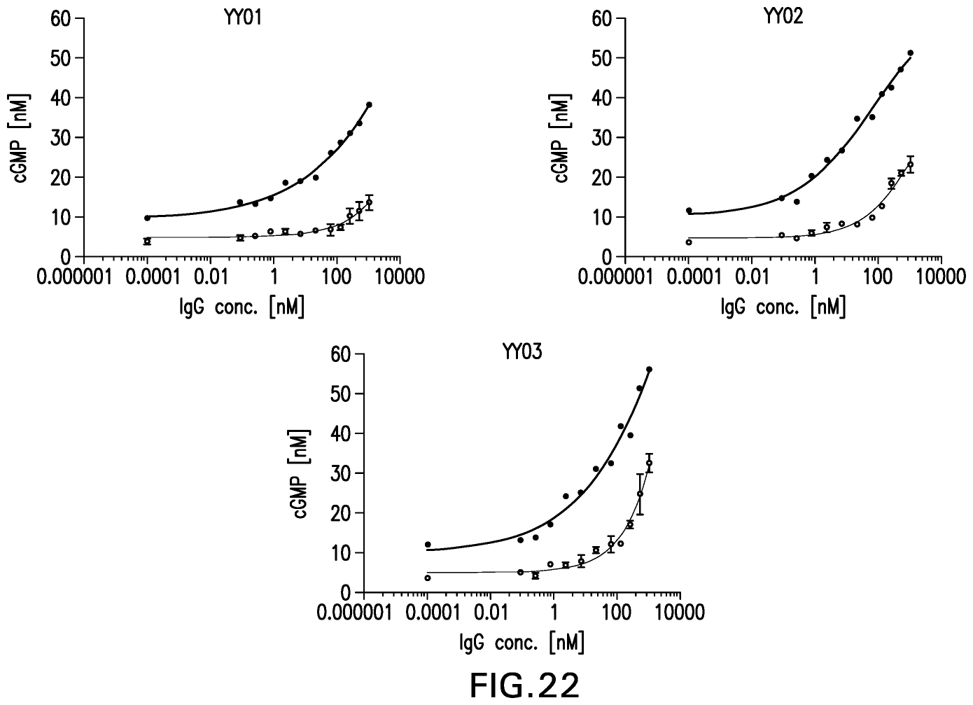

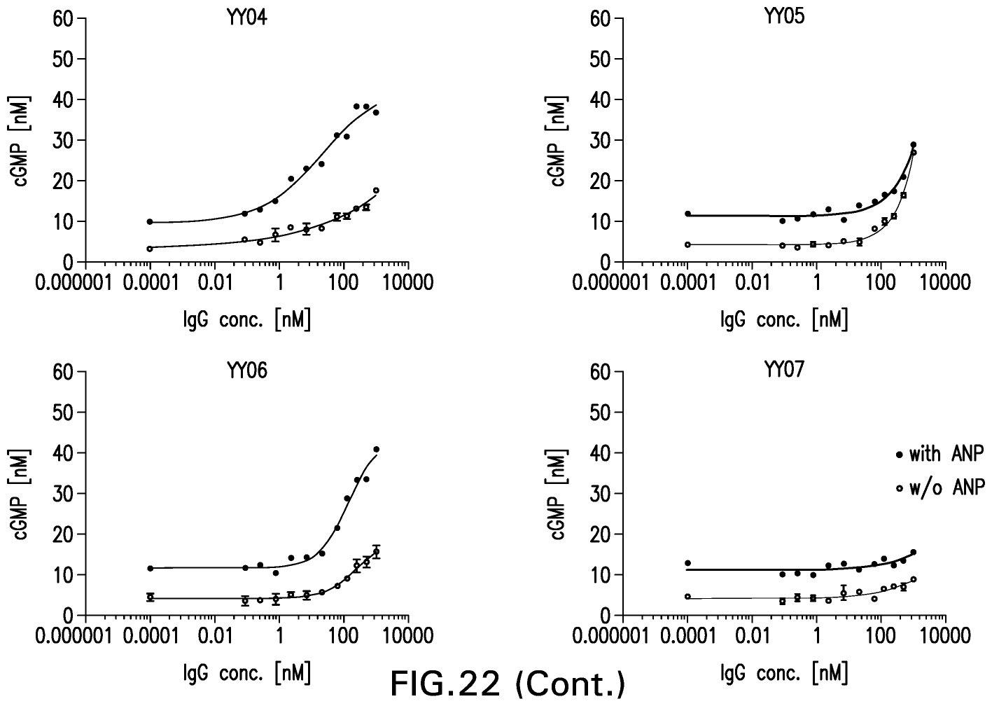

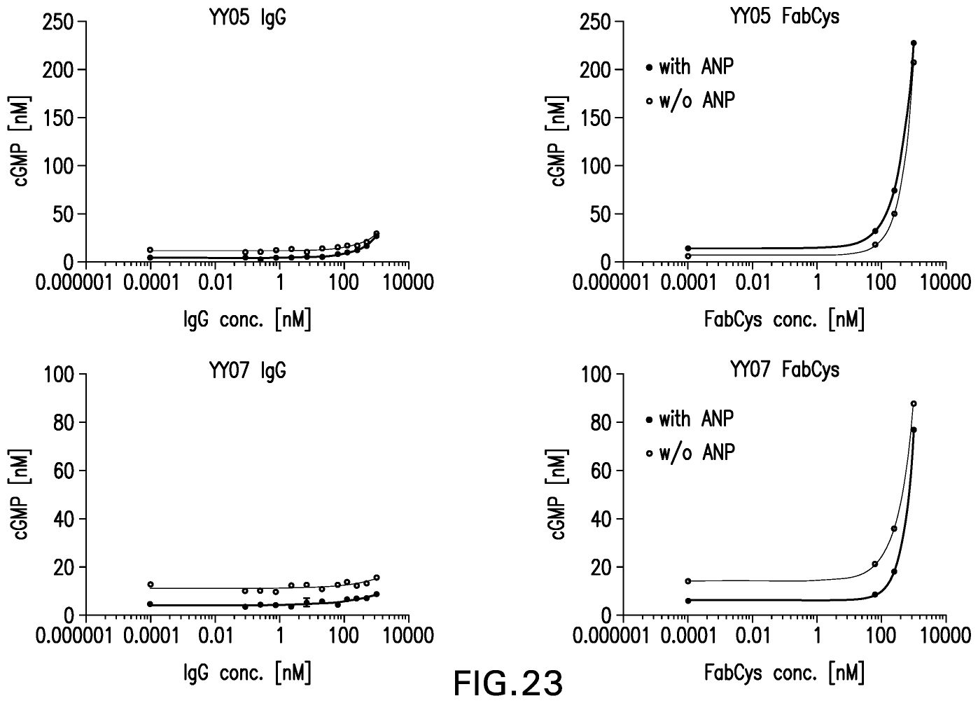

All 92 FabCys and 24 IgGs were tested for their functional activity in the cellular cGMP production assay using human NPR1 expressing CHO-K1 cells in presence and absence of ANP. 8 of the 92 FabCys and the same 8 out of 24 IgGs were functionally active and could be assigned to the different methods of action. Five out of eight clones showed much higher functional activity in presence of ANP, including YY01, YY02, YY03, and YY04. Three other clones behaved ANP independent in the functional assay, namely YY05, YY06, and YY07. All eight clones were derived from the panning strategies #2, #3 and #5, whereby #2 and #5 were exact repetitions of the initial HuCAL® panning strategies #10 and #11, which led to the identification of the five functional clones from the initial HuCAL® campaign. The results of the cGMP assay for seven functional candidates in IgG format are shown in FIG. 22. As seen before for candidate WW06 derived from the initial HuCAL® campaign, the functional activities of candidates YY05 and YY07 significantly increased in monovalent FabCys format compared to the bivalent IgG format (FIG. 23).

The seven functional candidates YY01-YY07 were analyzed with regard to their monovalent affinities for human and rat NPR1 in absence and presence of ANP in monovalent FabCys format. The results of the affinity determination and the epitope binning are summarized in Table 14.

TABLE 14
Characterization in monovalent FabCys format - Ylanthia ®

| | Characterization in monovalent FabCys format | | | | | | |
|---|---|---|---|---|---|---|---|
| | | | | | | Epitope | cGMP assay |
| | Affinity FabCys K_{D} [nM] | | | | | Binning | cGMP conc [nM] |
| | | | | | Bin on | FabCys | at 1 μM FabCys |
| | | hNPR1 + | | rNPR1 + | hNPR1(-ANP-com- | Without | +0.075 nM |
| Antibody | hNPR1 | ANP | rNPR1 | ANP | plex) | ANP | ANP |
| YY01 | — | 16 | — | 14 | B | 15 | 46 |
| YY02 | 45 | 1.1 | 280 | 4.4 | B | — | — |
| YY03 | — | 0.1 | 0.1 | 0.1 | B | — | — |
| YY04 | 4.9 | 1.1 | — | 1 | B | 18 | 67 |
| YY05 | 0.9 | 350 | 700 | 16000 | A | 207 | 227 |
| YY06 | 0.5 | 1 | 1600 | 1400 | C | 20 | 75 |
| YY07 | 1.8 | 590 | — | — | A | 77 | 88 |

The affinities were in the low nM to low μM range and strongly depended on the presence or absence of ANP. Four of the seven functional candidates showed significantly improved binding in presence of ANP (YY01, YY02, YY03, and YY04). YY05 and YY07 competed with ANP for binding to NPR1 and showed much higher affinities in absence of ANP. The affinities of YY06 were independent from ANP. Some candidates exhibited non-specific binding to the reference flow cell, while others had such high affinities that their K_{D} values approach the assay limit. YY01, YY02, YY03, and YY04 share one epitope bin, which is the same as for WW03 from the initial HuCAL® campaign. YY05 and YY07 share another epitope bin, which is the same as for WW06 from the initial HuCAL® campaign. YY06 binds to a single epitope bin.

### Example 12: Reformatting into IgG—Ylanthia®

Subcloning of the Ylanthia® candidates from the FabCys vector into the IgG1_LALA vector for expression in mammalian cells was performed via amplification of the Fab-encoding insert using two biotinylated primers. The amplified product was bound on streptavidin beads, digested using restriction enzymes, and washed, resulting in the release of the purified insert into the supernatant. The insert was cloned into the acceptor vector, the DNA was transformed and single clones were quality controlled via colony PCR and sequencing.

Five Ylanthia® candidates were selected for affinity maturation. YY01 and YY04 stabilize the NPR1-ANP-complex, YY06 behaves in an ANP-independent manner, and YY05 and YY07 are ANP-competitive. Binding data (ELISA, flow cytometry, ANP competition), functional data (cGMP assay), affinities, and epitope bins are shown in Table 15.

TABLE 15
Summary of Affinity, Epitope, and cGMP data for YY01, YY04, YY08, YY06, and YY07 in IgG Format

| | Characterization in bivalent IgG format | | | | | | | | | | | | |
|---|---|---|---|---|---|---|---|---|---|---|---|---|---|
| | | | | | | | | | Flow | | | | |
| | | | | | | | | | Cytometry | | ANP | | |
| | | | | | | | | | [S/BG] at | | compe- | cGMP assay | |
| | | | | | | | | | 1 μM IgG | | tition | cGMP | |
| | Elisa +/− 100 nM ANP IgG | | | | | | | | | hNPR1 | assay | conc [nM] | |
| | binding at 1 μM IgG | | | | | | | | | CHO | ANP | at 1 μM IgG | |
| | | hNPR1 + | | W74R + | | rNPR1 + | | hNPR3 _+ | hNPR1 | K1 + | compe- | | +0.075 |
| Anti- | | 100 nM | | 100 nM | | 100 nM | | 100 nM | CHO | 100 nM | tition | Without | nM |
| body | hNPR1 | ANP | W74R | ANP | rNPR1 | ANP | hNPR3 | ANP | K1 | ANP | by IgG | ANP | ANP |
| YY01 | 54 | 76 | 33 | 53 | 26 | 50 | 18 | 6 | 35 | 21 | yes | 5 | 14 |
| YY04 | 31 | 60 | 11 | 13 | 8 | 54 | 69 | 18 | 2 | 33 | no | 10 | 15 |
| YY05 | 81 | 32 | 69 | 23 | 11 | 16 | 7 | 4 | 15 | 5 | yes | 13 | 15 |
| YY06 | 91 | 102 | 83 | 86 | 3 | 4 | 6 | 6 | 26 | 18 | yes | 12 | 15 |
| YY07 | 68 | 24 | 53 | 20 | 3 | 3 | 6 | 5 | 8 | 44 | yes | 4 | 9 |

### Example 13: Generation of Ylanthia® Maturation Libraries

To increase affinity and biological activity and to reduce non-specificity of selected antibody candidates, LCDR3 and HCDR1/HCDR2 regions were optimized in parallel using diversified Ylanthia® maturation modules (YMM) previously generated with Slonomics® technology (van den Brulle et al. (2008): A novel solid phase technology for high-throughput gene synthesis; Biotechniques 45 (3), pp. 340-343, the contents of which are herein incorporated by reference for this purpose).

Cloning of the maturation libraries was performed in vectors encoding the parental Fab fragments. The generation of the maturation libraries was performed for five parental antibodies (YY01, YY04, YY05, YY06, and YY07). For the library generation, all maturation candidates were treated individually. The maturation libraries were successfully cloned and had library sizes between 6.2×10⁹ and 4.5×10⁹ cfu.

In order to monitor the cloning efficiency, the parental HCDR1/2 and LCDR3 were replaced by MBP-stuffers prior to insertion of the diversified YMM. Digested vector fragments were ligated with a 2-fold molar excess of the insert fragments carrying the diversified HCDR1/2 or LCDR3s. Ligation mixtures were electroporated in E. coli cells yielding in >10⁸ independent colonies. Amplification of the library was performed as described in the literature (Tiller et al. (2013): A fully synthetic human Fab antibody library based on fixed VH/VL framework pairings with favorable biophysical properties; mAbs 5 (3), pp. 445-470, the contents of which are herein incorporated by reference for this purpose). For quality control, approx. 10-20 single clones per library were randomly picked and sequenced.

### Example 14: Pannings and Screenings—Ylanthia®

The nine maturation libraries were used for four different maturation panning strategies, which aimed for the enrichment of progenies with improved affinities compared to the parental clones. Furthermore, rat material was included where appropriate and the pannings were performed with high stringencies concerning antigen concentration and washing conditions. During the panning process, the libraries of YY01 and YY04 (only LCDR3) as well as YY05 and YY07 were pooled, while the libraries of YY06 were kept separately. The panning strategies are summarized in Table 16 in detail. The bacterial lysates (BEL) of the outputs after the third panning rounds were directly used for an ELISA-based pre-screening and for SET screening.

TABLE 16
Overview of Ylanthia ® Maturation Panning Strategies

| | Parental | | | | |
|---|---|---|---|---|---|
| Strategy | antibodies | 1^{st} round | 2^{nd} round | 3^{rd} round | comments |
| 1 | YY01/ | hNPR1-ANP- | rNPR1-ANP- | hNPR1-ANP- | Solution panning aiming |
| | YY04 | complex solution | complex solution | complex solution | for enrichment of NPR1- |
| | | | | | ANP-complex stabilizers |
| 2 | YY06 | hNPR1-ANP- | hNPR1-ANP- | hNPR1-ANP- | Solution panning aiming |
| | | complex solution | complex solution | complex solution | for enrichment of NPR1- |
| | | | | | ANP-complex stabilizers |
| 3 | YY06 | Preadsorption on | Preadsorption on | Preadsorption on | Solution panning aiming |
| | YY05/ | NPR1-ANP-complex | NPR1-ANP-complex | NPR1-ANP-complex | for enrichment of ANP |
| | YY07 | hNPR1 solution | hNPR1 solution | hNPR1 solution | competitors |
| 4 | YY05/ | Preadsorption on | Preadsorption on | Preadsorption on | Solution panning aiming |
| | YY07 | NPR1-ANP-complex | NPR1-ANP-complex | NPR1-ANP-complex | for enrichment of ANP |
| | | hNPR1 solution | hNPR1 solution | INPRI solution | competitors |

The outputs of the third panning rounds were used for an ELISA-based pre-screening to ensure that only clones binding to NPR1 and/or NPR1-ANP-complex were selected for further Solution Equilibrium Titration (SET) screening. 880 clones in total were analyzed in SET screening for improved affinity for hNPR1 and/or hNPR1-ANP-complex compared to the parental clones.

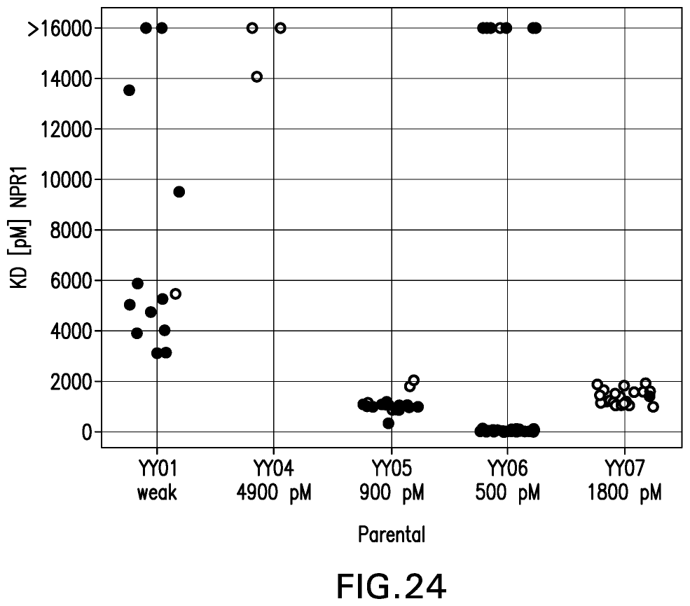

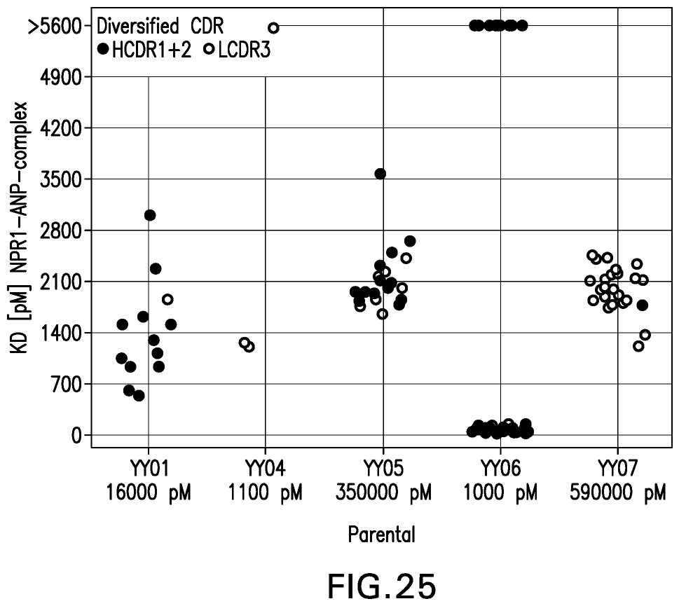

During SET screening, 263 HCDR1/2 or LCDR3 unique improved derivatives were identified, which resulted in 112 unique clones after sequencing and conversion to IgG1 LALA format. Compared to their parental clones, the affinities of the YY05 and YY07 derivatives were not improved for NPR1 but were improved up to 200-fold for NPR1-ANP-complex. The derivatives of YY01 had similar affinities to the parental clone YY04, whose derivatives had only slightly improved affinities. The affinities of the YY06 derivatives were improved 4- to 40-fold for NPR1 and 7- to 70-fold for NPR1-ANP-complex. See FIG. 24 (which shows affinities for hNPR1) and FIG. 25 (which shows affinities for hNPR1-ANP complex). Affinities (K_{D} [pM]) are indicated at the x-axis below the parental clone name for both figures.

### Example 15: Characterization of Matured Candidates—Ylanthia®

95 of 112 improved candidates were selected for advanced production. 77 of the 95 clones passed the production quality control and were characterized in regard to binding to relevant antigens, binding to relevant cell lines, and functional activity in the cGMP production assay in comparison to their parental clones. After detailed IgG characterization, 17 candidates (detailed in Table 17) were selected, produced in exploratory scale IgG production, and further analyzed via 3P assay. Furthermore, they were converted to FabCys format for their affinity determination on human and rat NPR1 in absence and presence of ANP via SET K_{D} measurement.

TABLE 17
Overview of Matured Ylanthia ® Candidates

| Matured | Parental | |
|---|---|---|
| antibody | antibody | Matured CDR |
| ZZ01 | YY07 | LCDR3 |
| ZZ02 | YY05 | LCDR3 |
| ZZ03 | YY07 | LCDR3 |
| ZZ04 | YY07 | LCDR3 |
| ZZ05 | YY07 | LCDR3 |
| ZZ06 | YY07 | LCDR3 |
| ZZ07 | YY07 | LCDR3 |
| ZZ08 | YY07 | LCDR3 |
| ZZ09 | YY05 | HCDR1 + 2 |
| ZZ10 | YY05 | HCDR1 + 2 |
| ZZ11 | YY04 | LCDR3 |
| ZZ12 | YY01 | HCDR1 + 2 |
| ZZ13 | YY01 | HCDR1 + 2 |
| ZZ14 | YY06 | HCDR1 + 2 |
| ZZ15 | YY06 | HCDR1 + 2 |
| ZZ16 | YY06 | HCDR1 + 2 |
| ZZ17 | YY06 | HCDR1 + 2 |

Affinities for 16 of the matured Ylanthia® candidates (in monovalent FabCys format) were determined via SET measurement and/or via Octet. The results are summarized in Tables 18 and 19 below in comparison to the affinities of the parental clones.

TABLE 18
Summary of Affinity data (SET) for YY01, YY06, ZZ12, ZZ13, ZZ15, ZZ16, and
ZZ17 in FabCys Format

| | Affinity (SET measurement) | | | | |
|---|---|---|---|---|---|
| | FabCys K_{D} [nM] | | | | |
| | | hNPR1 + | | rNPR1 + | |
| Antibody | hNPR1 | ANP | rNPR1 | ANP | comment |
| YY01 | — | 16.00 | — | 14.00 | K_{D} determination via Octet |
| ZZ12 | 6.70 | 0.32 | 2.10 | 1.40 | |
| ZZ13 | 9.90 | 1.30 | 2.30 | 1.40 | |
| YY04 | 4.90 | 1.10 | — | 1.00 | K_{D} determination via Octet |
| ZZ11 | 46.00 | 2.40 | 21.00 | 18.00 | |
| YY05 | 0.90 | 350.00 | 700.00 | 16000.00 | K_{D} determination via Octet; |
| | | | | | assay limit reached |
| ZZ02 | 0.37 | 0.58 | — | — | |
| ZZ09 | 0.49 | 0.57 | — | — | |
| ZZ10 | 0.52 | 1.40 | — | — | |
| YY06 | 0.50 | 1.00 | 1600.00 | 1400.00 | K_{D} determination via Octet |
| ZZ15 | 0.053 | 0.074 | — | — | |
| ZZ16 | 0.040 | 0.034 | 62.00 | 51.00 | |
| ZZ17 | 0.046 | 0.050 | 52.00 | 36.00 | |
| YY07 | 1.80 | 590.0 | — | — | |
| ZZ01 | 0.65 | 0.85 | — | — | |
| ZZ03 | 0.75 | 1.30 | — | — | |
| ZZ04 | 0.30 | 0.38 | — | — | |
| ZZ05 | 0.74 | 1.20 | — | — | |
| ZZ06 | 0.35 | 0.50 | — | — | |
| ZZ07 | 0.62 | 1.50 | — | — | |
| ZZ08 | 0.57 | 1.30 | — | — | |

TABLE 19
Summary of Affinity data (Octet) for YY01, YY06, ZZ12,
ZZ13, ZZ15, ZZ16, and ZZ17 in FabCys Format

| | Affinity (Octet measurement) | | | | |
|---|---|---|---|---|---|
| | FabCys K_{D} [nM] | | | | |
| | | hNPR1 + | | rNPR1 + | |
| Antibody | hNPR1 | ANP | rNPR1 | ANP | comment |
| YY01 | — | 26.00 | — | 8.80 | Heterogenous |
| ZZ12 | — | 0.04 | — | 0.10 | binding |
| ZZ13 | — | 0.14 | 66.00 | 0.10 | |
| YY06 | 0.62 | 0.80 | 100.00 | 38.00 | |
| ZZ15 | 0.26 | 0.19 | — | — | |
| ZZ16 | 0.14 | 0.11 | — | — | |
| ZZ17 | 0.16 | 0.09 | — | — | |

### Example 16: Epitope Determination for ANP Competitive and ANP Non-Competitive Agonist Antibodies

The crystal structure of the hNPR1 and XX 16 Fab complex was used to identify the XX16 Fab epitope on hNPR1. The interaction surface on hNPR1 by XX16 Fab was formed by several continuous and discontinuous (i.e., noncontiguous) sequences as detailed in Table 20. These residues form the three-dimensional conformational epitope recognized by XX16 Fab.

Results of the epitope mapping of XX16 Fab (ANP non-competitive) are shown in Table 20. hNPR1 residues are numbered based upon SEQ ID NO: 1 (P16066). Fab residues are numbered based upon their linear amino acid sequence. hNPR1 residues shown have at least one atom within 5 Å of an atom in the XX16 Fab, to account for potential water mediated interactions.

TABLE 20
Epitope Mapping of XX16 Fab

| Antibody XX16 Fab | | | Antigen (NPR1) | | |
|---|---|---|---|---|---|
| Amino acid | Number | Chain | Amino acid | Number | Chain |
| SER | 31 | H | TRP | 106 | A |
| TYR | 32 | H | TRP | 106 | A |
| | | | TRP | 106 | B |
| TRP | 33 | H | ASN | 109 | A |
| GLU | 52 | H | HIS | 108 | A |
| | | | ASN | 109 | A |
| SER | 53 | H | GLU | 107 | A |
| | | | ASN | 109 | A |
| LYS | 54 | H | GLU | 107 | A |
| ASN | 56 | H | GLY | 33 | A |
| | | | ASN | 34 | A |
| | | | THR | 36 | A |
| | | | TRP | 76 | A |
| TYR | 57 | H | ASN | 34 | A |
| | | | TRP | 76 | A |
| | | | ASN | 109 | A |
| | | | PRO | 110 | A |
| | | | ALA | 111 | A |
| | | | VAL | 374 | A |
| | | | THR | 375 | A |
| PHE | 59 | H | THR | 375 | A |
| ARG | 100 | H | TRP | 106 | A |
| | | | ASP | 103 | B |
| | | | GLU | 107 | B |
| TYR | 101 | H | VAL | 102 | A |
| | | | TRP | 106 | A |
| | | | VAL | 102 | B |
| | | | ASP | 103 | B |
| | | | TRP | 106 | B |
| | | | GLU | 107 | B |
| SER | 102 | H | LYS | 105 | A |
| | | | TRP | 106 | A |
| MET | 103 | H | VAL | 102 | A |
| | | | LYS | 105 | A |
| | | | TRP | 106 | A |
| | | | TRP | 132 | A |
| | | | LEU | 99 | B |
| | | | VAL | 102 | B |
| | | | ASP | 103 | B |
| ILE | 104 | H | LYS | 105 | A |
| | | | HIS | 131 | A |
| | | | TRP | 132 | A |
| | | | LEU | 99 | B |
| | | | ALA | 100 | B |
| | | | ASP | 103 | B |
| TYR | 105 | H | LYS | 105 | A |
| | | | TRP | 132 | A |
| SER | 106 | H | LYS | 105 | A |
| | | | HIS | 131 | A |
| | | | TRP | 132 | A |
| | | | ARG | 133 | A |
| TYR | 107 | H | LYS | 105 | A |
| | | | ASN | 109 | A |
| | | | PRO | 110 | A |
| | | | ALA | 111 | A |
| | | | VAL | 134 | A |
| | | | THR | 375 | A |
| ASP | 113 | H | GLU | 107 | B |
| ARG | 18 | L | GLN | 336 | B |
| SER | 30 | L | GLU | 86 | B |
| | | | ASN | 87 | B |
| | | | ALA | 88 | B |
| SER | 31 | L | SER | 84 | B |
| | | | GLU | 86 | B |
| TYR | 32 | L | HIS | 131 | A |
| TYR | 49 | L | ASP | 103 | B |
| | | | LEU | 104 | B |
| | | | GLU | 107 | B |
| | | | HIS | 108 | B |
| SER | 52 | L | LEU | 43 | B |
| | | | LEU | 82 | B |
| THR | 53 | L | VAL | 81 | B |
| | | | LEU | 82 | B |
| | | | GLY | 83 | B |
| | | | LEU | 104 | B |
| | | | HIS | 108 | B |
| LEU | 54 | L | ARG | 79 | B |
| | | | VAL | 81 | B |
| | | | HIS | 108 | B |
| GLN | 55 | L | GLU | 107 | B |
| | | | HIS | 108 | B |
| SER | 56 | L | GLU | 107 | B |
| | | | HIS | 108 | B |
| VAL | 58 | L | ARG | 79 | B |
| PRO | 59 | L | ARG | 79 | B |
| SER | 60 | L | ARG | 79 | B |
| SER | 65 | L | THR | 44 | B |
| GLY | 66 | L | THR | 44 | B |
| SER | 67 | L | THR | 44 | B |
| | | | GLU | 86 | B |
| | | | ASN | 87 | B |
| SER | 76 | L | GLN | 336 | B |
| TRP | 92 | L | HIS | 131 | A |
| | | | ARG | 133 | A |
| | | | ALA | 88 | B |
| ARG | 93 | L | ARG | 133 | A |
| | | | GLU | 378 | A |
| LYS | 94 | L | ASP | 376 | A |

Critical epitope residues for the binding of XX16 Fab and NPR1, which were determined using structural analysis and affinity maturation data, include (first tier) 99-103, 105-111, 131-133, 378; and (second tier): 33-34, 76, 82, 104, 374-375. Amino acids 99-103 of NPR1 (SEQ ID NO: 1) comprise both a E106 backbone that enabled the affinity maturation by D54K in HCDR2, and W106 from both NPR protomers that clamp Y101 of HCDR3. Certain critical epitope residues are shown in Table 21 below. Regions of NPR1 encompassing these critical residues include N34, W76, L82, V102-A111, H131-V134, and V374-E378.

TABLE 21
Critical Epitope Residues of XX16 Fab (ANP non-competitive)

| Critical epitope | | |
|---|---|---|
| residue (amino | Critical epitope | |
| acid) | residue (number) | Explanation |
| N | 34 | H-bond with N56 (backbone) of VH |
| L | 82 | H-bond (chain B) with T53 (side chain) of VL |
| V | 102 | Packing against M103 (side chain) of VH |
| D | 103 | Salt bridge (chain B) with R100 (side chain) of VH |
| K | 105 | H-bond (backbone) with M103 (backbone) and Y105 (backbone) of VH |
| W | 106 | Packing between Y32 (side chain) and Y101 (side chain) of VH |
| E | 107 | Salt bridge (chain B) with R100 (side chain) of VH |
| E | 107 | H-bond (backbone) with K54 (side chain) of VH |
| N | 109 | H-bond with E52 (side chain), S53 (side chain) and W33 (side chain) of VH |
| W | 132 | Packing against M103 (side chain) of VH |
| R | 133 | H-bond with S106 (side chain) of VH |
| T | 375 | Packing against Y57 (side chain) of VH |
| E | 378 | Salt bridge with R93 (side chain) of VL |

The crystal structure of the hNPR1 and WW03 Fab complex was used to identify the WW03 Fab epitope on hNPR1. The interaction surface on hNPR1 by WW03 Fab was formed by several continuous and discontinuous (i.e., noncontiguous) sequences as detailed in Table 22. These residues form the three-dimensional conformational epitope recognized by WW03 Fab.

Results of the epitope mapping of WW03 Fab (ANP non-competitive) are shown in Table 22. hNPR1 residues are numbered based upon SEQ ID NO: 1 (P16066). Fab residues are numbered based upon their linear amino acid sequence. hNPR1 residues shown have at least one atom within 5 Å of an atom in the WW03 Fab, to account for potential water mediated interactions.

TABLE 22
Epitope Mapping of WW03 Fab

| Antibody WW03 Fab | | | Antigen (NPR1) | | |
|---|---|---|---|---|---|
| Amino acid | Number | Chain | Amino acid | Number | Chain |
| SER | 31 | H | TRP | 106 | A |
| TYR | 32 | H | TRP | 106 | A |
| | | | TRP | 106 | B |
| TRP | 33 | H | ASN | 109 | A |
| SER | 52 | H | HIS | 108 | A |
| | | | ASN | 109 | A |
| SER | 53 | H | TRP | 106 | A |
| | | | GLU | 107 | A |
| | | | ASN | 109 | A |
| ASP | 54 | H | THR | 36 | A |
| | | | ARG | 79 | A |
| SER | 56 | H | ASN | 34 | A |
| TYR | 57 | H | ASN | 109 | A |
| | | | PRO | 110 | A |
| | | | ALA | 111 | A |
| | | | VAL | 374 | A |
| | | | THR | 375 | A |
| TYR | 59 | H | THR | 375 | A |
| ARG | 100 | H | ASP | 103 | B |
| | | | TRP | 106 | B |
| | | | GLU | 107 | B |
| TYR | 101 | H | VAL | 102 | A |
| | | | TRP | 106 | A |
| | | | VAL | 102 | B |
| | | | ASP | 103 | B |
| | | | TRP | 106 | B |
| | | | GLU | 107 | B |
| SER | 102 | H | LYS | 105 | A |
| | | | TRP | 106 | A |
| | | | ASN | 109 | A |
| MET | 103 | H | VAL | 102 | A |
| | | | LYS | 105 | A |
| | | | TRI | 106 | A |
| | | | TRP | 132 | A |
| | | | LEU | 99 | B |
| | | | VAL | 102 | B |
| | | | ASP | 103 | B |
| ILE | 104 | H | HIS | 131 | A |
| | | | TRP | 132 | A |
| | | | LEU | 99 | B |
| | | | ASP | 103 | B |
| TYR | 105 | H | LYS | 105 | A |
| | | | TRP | 132 | A |
| SER | 106 | H | LYS | 105 | A |
| | | | TRP | 132 | A |
| | | | ARG | 133 | A |
| TYR | 107 | H | LYS | 105 | A |
| | | | ASN | 109 | A |
| | | | PRO | 110 | A |
| | | | ALA | 111 | A |
| | | | VAL | 134 | A |
| | | | THR | 375 | A |
| ASP | 113 | H | GLU | 107 | B |
| ARG | 18 | L | GLN | 336 | B |
| SER | 30 | L | ASN | 87 | B |
| | | | ALA | 88 | B |
| SER | 31 | L | SER | 84 | B |
| TYR | 32 | L | HIS | 131 | A |
| TYR | 49 | L | ASP | 103 | B |
| | | | GLU | 107 | B |
| | | | HIS | 108 | B |
| SER | 52 | L | LEU | 43 | B |
| | | | LEU | 82 | B |
| THR | 53 | L | VAL | 81 | B |
| | | | LEU | 82 | B |
| | | | GLY | 83 | B |
| | | | LEU | 104 | B |
| | | | HIS | 108 | B |
| LEU | 54 | L | ARG | 79 | B |
| | | | VAL | 81 | B |
| | | | HIS | 108 | B |
| GLN | 55 | L | GLU | 107 | B |
| | | | HIS | 108 | B |
| SER | 56 | L | GLU | 107 | B |
| | | | HIS | 108 | B |
| VAL | 58 | L | ARG | 79 | B |
| PRO | 59 | L | ARG | 79 | B |
| SER | 60 | L | ARG | 79 | B |
| GLY | 66 | L | THR | 44 | B |
| SER | 67 | L | THR | 44 | B |
| | | | GLU | 86 | B |
| | | | ASN | 87 | B |
| TYR | 92 | L | ARG | 133 | A |
| GLU | 93 | L | ARG | 133 | A |
| LYS | 94 | L | ASP | 376 | A |

Critical epitope residues for the binding of WW03 Fab and NPR, which were determined using structural analysis and affinity maturation data, include the residues shown in Table 23. Regions of NPR1 encompassing these critical residues include R79, L82, L99-A111, H131-V134, and V374-T375.

TABLE 23
Critical Epitope Residues of WW03 Fab (ANP non-competitive)

| | Critical | |
|---|---|---|
| Critical epitope | epitope | |
| residue (amino | residue | |
| acid) | (number) | Explanation |
| R | 79 | Salt bridge with D54 (side chain) of VH; H-bond (chain B) with S60 (side chain) of VL |
| L | 82 | H-bond (backbone) with T53 (side chain) of VL |
| L | 99 | Packing against M103 (side chain) and 1104 (side chain) of VH |
| V | 102 | Packing against M103 (side chain) of VH |
| D | 103 | Salt bridge with R100 (side chain) of VH |
| K | 105 | H-bond with M103 (backbone) and Y105 (backbone) of VH |
| W | 106 | Packing between Y32 (side chain) and Y101 (side chain) of VH; stacking (chain B) |
| | | against Y101 (side chain) of VH |
| N | 109 | H-bond with S53 (side chain) of VH |
| H | 131 | H-bond with I104 (backbone) of VH |
| W | 132 | H-bond with M103 (side chain) of VH |
| T | 375 | H-bond with Y59 (side chain) of VH; pack against Y57 (side chain) of VH |

The crystal structure of the hNPR1 and WW06 Fab complex was used to identify the WW06 Fab epitope on hNPR1. The interaction surface on hNPR1 by WW06 Fab was formed by several continuous and discontinuous (i.e., noncontiguous) sequences as detailed in Table 24. These residues form the three-dimensional conformational epitope recognized by WW06 Fab.

Results of the epitope mapping of the WW6 Fab (ANP competitive) are shown in Table 24. hNPR1 residues are numbered based upon SEQ ID NO: 1 (P16066). WW06 Fab residues are numbered based upon their linear amino acid sequence. hNPR1 residues shown have at least one atom within 5 Å of an atom in the WW06 Fab, to account for potential water mediated interactions.

TABLE 24
Epitope Mapping of WW06 Fab (ANP competitive)

| Antibody WW06 Fab | | | Antigen (NPR1) | | |
|---|---|---|---|---|---|
| Amino acid | Number | Chain | Amino acid | Number | Chain |
| Y | 27 | H | R | 294 | B |
| | | H | G | 295 | B |
| S | 28 | H | R | 294 | B |
| | | H | G | 295 | B |
| | | H | D | 296 | B |
| S | 31 | H | R | 230 | A |
| | | H | D | 296 | B |
| Y | 32 | H | G | 295 | B |
| | | H | D | 296 | B |
| | | H | G | 297 | B |
| W | 33 | H | Y | 188 | A |
| | | H | E | 219 | A |
| R | 50 | H | F | 197 | A |
| | | H | E | 219 | A |
| D | 52 | H | Y | 188 | A |
| | | H | A | 221 | A |
| | | H | H | 227 | A |
| D | 54 | H | D | 224 | A |
| N | 55 | H | Y | 188 | A |
| | | H | P | 190 | A |
| | | H | A | 221 | A |
| | | H | D | 223 | A |
| Y | 57 | H | Y | 188 | A |
| | | H | R | 189 | A |
| | | H | P | 190 | A |
| | | H | G | 191 | A |
| | | H | D | 192 | A |
| | | H | E | 193 | A |
| | | H | E | 194 | A |
| | | H | F | 197 | A |
| T | 58 | H | E | 194 | A |
| R | 59 | H | E | 194 | A |
| | | H | F | 197 | A |
| | | H | E | 201 | A |
| Q | 65 | H | D | 94 | A |
| | | H | E | 194 | A |
| R | 98 | H | G | 295 | B |
| W | 99 | H | E | 219 | A |
| L | 100 | H | R | 230 | A |
| S | 101 | H | H | 217 | A |
| P | 102 | H | M | 238 | A |
| 0 | 103 | H | D | 216 | A |
| | | H | H | 217 | A |
| | | H | H | 234 | A |
| | | H | K | 238 | A |
| Y | 104 | H | L | 218 | A |
| | | H | E | 219 | A |
| | | H | F | 220 | A |
| | | H | H | 227 | A |
| | | H | R | 230 | A |
| | | H | L | 231 | A |
| | | H | H | 234 | A |
| | | H | K | 238 | A |
| A | 105 | H | K | 238 | A |
| L | 106 | H | R | 233 | 4 |
| | | H | T | 234 | A |
| | | H | R | 237 | A |
| | | H | K | 238 | A |
| G | 107 | H | R | 237 | A |
| Q | 109 | H | G | 297 | B |
| Q | | H | V | 300 | B |
| I | 29 | L | R | 208 | A |
| G | 30 | L | R | 208 | A |
| A | 31 | L | F | 204 | A |
| | | L | R | 208 | A |
| G | 32 | L | R | 208 | A |
| Y | 33 | L | Y | 186 | A |
| | | L | E | 201 | A |
| | | L | F | 204 | A |
| | | L | H | 217 | A |
| Y | 93 | L | F | 197 | A |
| | | L | E | 201 | A |
| L | 95 | L | L | 144 | A |
| | | L | E | 201 | 4 |
| | | L | F | 204 | A |
| | | L | M | 205 | A |
| Q | 96 | L | L | 144 | A |
| | | L | G | 145 | A |
| | | L | V | 148 | A |
| | | L | M | 205 | A |
| S | 98 | L | F | 197 | A |
| | | L | F | 198 | A |
| | | L | E | 201 | A |
| R | 100 | L | E | 219 | A |

Critical epitope residues for the binding of WW06 and NPR1, which were determined using structural analysis and affinity maturation data, include the residues shown in Table 25. Regions of NPR1 encompassing these critical residues include Y188-F198, E201-R208, V215-K238, and R294-G297.

TABLE 25
Critical Epitope Residues of WW06 (ANP competitive)

| Critical epitope | | |
|---|---|---|
| residue (amino | Critical epitope | |
| acid) | residue (number) | Explanation |
| Y | 188 | Packing against W33 (side chain) of VH |
| D | 192 | H-bond with Y57 (side chain) of VH |
| E | 194 | Salt bridge with R59 (side chain) of VH |
| F | 197 | Stacking against R59 (side chain) of VH |
| E | 201 | H-bond with S98 (side chain) of VL |
| R | 208 | H-bond with G30 (backbone) of VL |
| E | 219 | Salt bridge with R50 (side chain) of VH |
| G | 295 | H-bond (backbone, chain B) with S28 (backbone) of VH |

### Example 17: Mouse In Vivo Characterization of Effect of WW06 on Plasma cGMP

WW06 FabCys was used in an in vivo study in hNPR1 transgenic mice to determine the effect of this antibody on plasma cGMP levels in vivo.

For analysis of plasma cGMP samples, the LC-MS/MS detection method using ¹⁵N₂, ¹³C cGMP as an internal standard was adopted from Oeckl and Ferger, Journal of Neuroscience Methods 203 (2012) 338-343; and Zhang et al., J. Chromatogr B:Analyt Technol Biomed Life Sci 2009; 877:513-20; the contents of each of which are hereby incorporated by reference for this purpose).

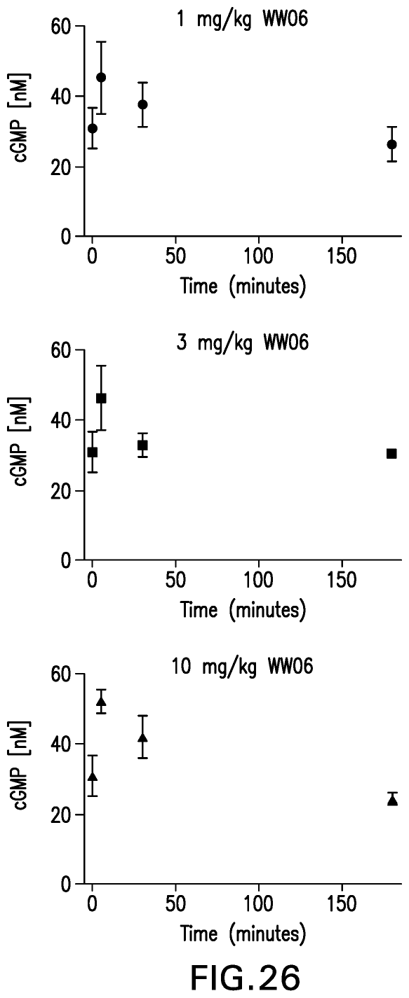

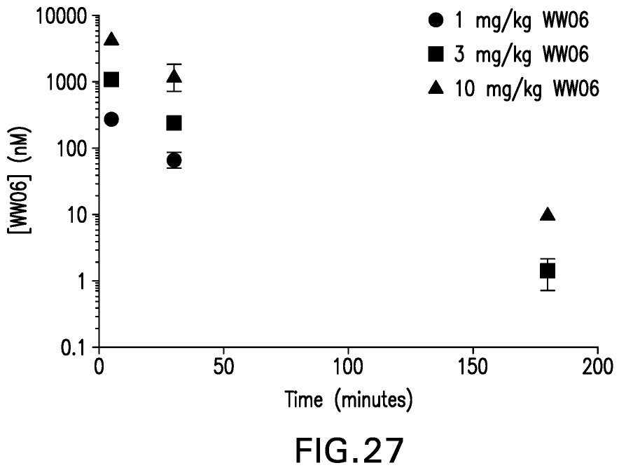

Plasma cGMP concentration in hNPR1 Tg mice which were intravenously administered the WW06 Fab increased at the 5 minute time point and the signal returned to baseline by 3 h. As expected and consistent with the data shown in FIGS. 26 and 27, the T_{1/2} of the FabCys antibody was <30 min. Each value shown is the average of three points collected from three individual animals. Dose response data are shown in FIG. 26, and PK data are shown in FIG. 27.

### Example 18: Effect of XX16 on Heart Weight and NT-proBNP in ANP KO and WT Mice

XX16 was used in an in vivo study to determine its effect on heart weight and NT-proBNP levels in ANP knockout (KO) and wild-type (WT) mice.

ANP knockout mice are hypertensive and have cardiac hypertrophy (increased HW/BW ratio). NT-proBNP is a biomarker of cardiac dysfunction. XX16 was administered at 0.3 or 3 mg/kg subcutaneously once every two weeks for four weeks in ANP knockout and wild type mice.

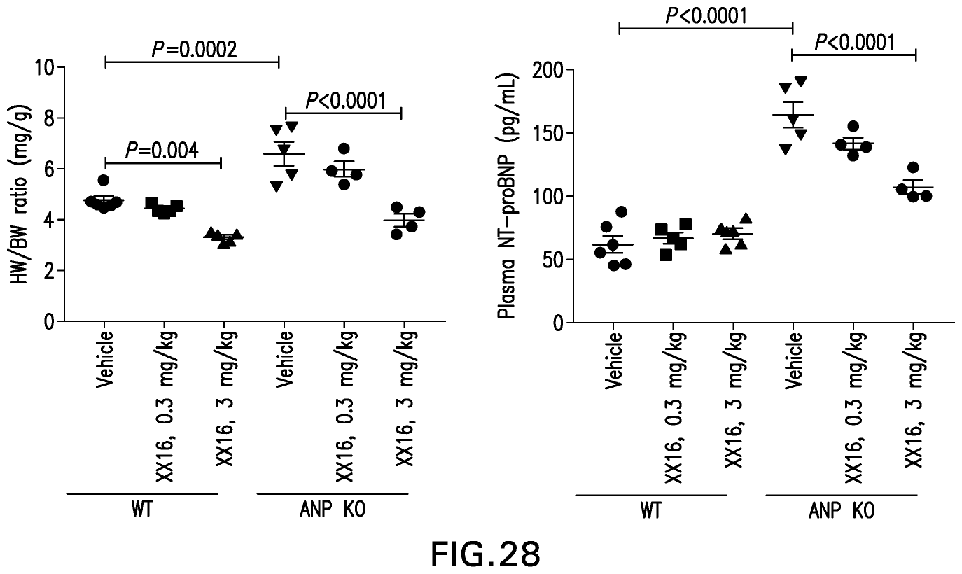

Results are shown in FIG. 28. At two weeks after the second treatment, XX16 dose-dependently reduced heart weight/body weight ratio and NT-proBNP in both wild type and ANP KO mice in comparison to vehicle treated animals.

### Example 19: Acute Effect of XX16 on Blood Pressure and Urinary Flow Rate in Hypertensive Rats

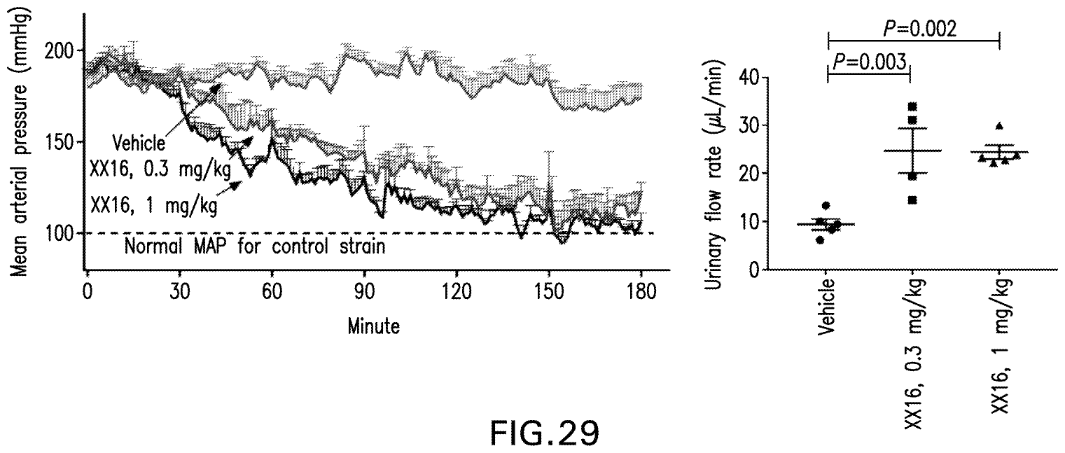

XX16 was used to determine its effect on blood pressure and urinary flow rate in hypertensive rats (spontaneous hypertensive rat stroke prone, SHRsp). Animals were administered 0.3 mg/kg XX16, 1 mg/kg XX16, or a vehicle control intravenously (one time). Blood pressure was measured using a femoral artery catheter. Measurements were taken three hours after the intravenous administration and results are shown in FIG. 29.

Intravenous XX16 treatment normalized mean arterial pressure and increased urinary flow rate acutely in hypertensive rats (SHRsp) in comparison to vehicle treated animals.

### Example 20: Chronic Effects of XX16 on Blood Pressure and Plasma cGMP in Wistar-Han Rats

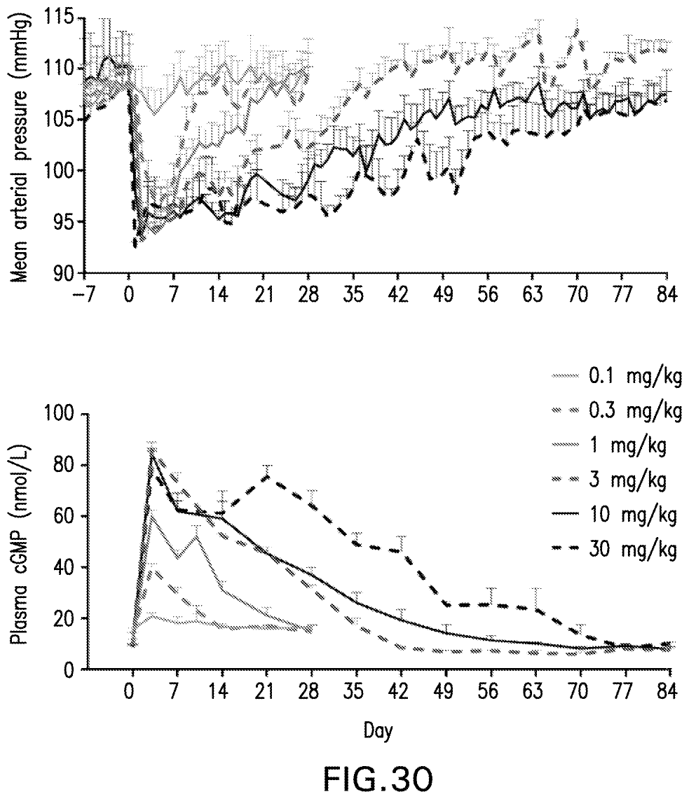

Chronic hemodynamic effects of XX16 in telemetry implanted normal rats were evaluated. XX16 (at doses of 0.1, 0.3, 1, 3, 10, and 30 mg/kg) was administered subcutaneously one time. Results are shown in FIG. 30. Subcutaneous administration of XX16 decreased mean arterial pressure (MAP) in all groups except for the 0.1 mg/kg group. There was a floor (around 95 mmHg) for the MAP reduction. A dose-dependent effect for the time to return baseline MAP was observed; this effect was greater than 70 days for the 30 mg/kg treatment group. Plasma cGMP concentration also had a ceiling around 90 nmol/L, after which the plasma cGMP gradually reduced toward baseline. This effect is very similar to the blood pressure response.

### Example 21: Human Fab Phage Library Panning

Phage Display panning. Antibodies against XX16 were generated by the selection of clones that bound to XX16 using as a source of antibody a phage display library built out of human germline framework combinations. Only CDR-H3 was diversified and primers were designed to incorporate all naturally occurring amino acids excluding cysteine, methionine, and asparagine using trinucleotide technology (ELLA Biotech). CDR-H3 lengths between 10 and 20 amino acids were allowed, in which the last two amino acids were kept constant with the sequence Asp-Tyr for length 10 to 16 and Asp-Val for length 18 and 20. The design of the final two CDR-H3 amino acids reflects human VDJ recombination. For the isolation of anti-XX16 antibodies a solid phase panning strategy was employed with direct coating of XX16 to immunotubes (Nunc) followed by three rounds of panning with increasing washing stringency. The third panning round was performed in order to select anti-XX16 specific antibodies which did not bind to an irrelevant hIgG1 sharing the same germline framework combination as XX16. This round was based on the output of the second round panning on XX16 and performed on 2 different antigens: XX16 and an irrelevant hIgG1. The output of this third analytical round underwent a Next Generation Sequencing (NGS) analysis.

Next Generation Sequencing (NGS) Analysis. The DNA of the third panning round was extracted and the HCDR3 region was amplified in a PCR reaction. The PCR reaction was also used to add Illumina adaptor sequences to the 3′ and the 5′ end of the PCR fragment. Additionally, Illumina indexes were added in one adapter region in order to multiplex the samples for the sequencing reaction. The raw data in FastQ format were used to extract amino acid sequences, align the sequences, and count the occurrence of individual sequences. By comparing occurrences of individual clones deriving from different panning strategies, clones with desired binding profile (enriched on XX16 and depleted on irrelevant hIgG1) could be identified. Amino acid CDR-H3 sequences of interesting clones were extracted from the raw data and genes of heavy and light chains were synthesized (GeneArt).

IgG Expression. In order to express the candidates in human IgG1 format Expi293F™ cells and appropriate vectors for human IgG1 expression synthesized by GeneArt were used. The cell culture supernatant was harvested 5 days post transfection. After sterile filtration, the solution was subjected to Protein A affinity chromatography using a Tecan system. Samples were eluted in 50 mM NaH₂PO₄, 100 mM NaCl, pH 3.0. pH was neutralized with 0.5M Na₂HPO₄ pH 9.0. Samples in final buffer 137 mM Na-phosphate 81 mM NaCl pH 7.0 were sterile filtered (0.2 μm pore size). Protein concentrations were determined by HPLC and purity of IgGs was analyzed by SEC.

Fab Expression. In order to express the candidates in human Fab format HEK293-T cells and appropriate vectors for human Fab expression synthesized by GeneArt were used. The cell culture supernatant was harvested 7 days post transfection. After sterile filtration, the solution was subjected to CH1 affinity chromatography using a Tecan system. Samples were eluted in 0.1M Glycine pH2.7 and buffer was then exchanged using PD10 columns. Samples in final buffer dPBS pH7.3 were sterile filtered (0.2 μm pore size). Protein concentrations were determined by HPLC and purity of Fabs was analyzed by SEC.

### Example 22: Binding Analysis of Anti-XX16 Fab Binding to XX16

All binding experiments were performed on an Octet® instrument (FortéBio) in 1× Octet® Kinetics buffer (1×KB) (FortéBio) at 25° C. XX16 was immobilized using anti-hIgG Fc Capture (AHC) biosensor tips (FortéBio) and binding interactions were recorded for anti-XX16 Fabs as analytes in solution as described by the manufacturer.

XX16 prepared at 50 nM in 1×KB was dispensed in 8 vertical wells of a Greiner BioOne PP 96 well micro plate at a volume of 200 μl per well. Six analyte concentration from 0 nM to 100 nM (prepared as 3-fold serial dilutions) were generated for each anti-XX16 Fab (analyte) in 1×KB and dispensed in vertical wells of the same 96 well micro plate at a volume of 200 μl per well. Another column of vertical wells were filled with 1×KB buffer. AHC biosensors were hydrated in 200 μl of 1×KB for 10 min. For kinetic studies, AHC tips were dipped for 60 sec in 1×KB to establish a baseline and then transferred to XX16-containing wells for a 180-sec loading step. After a 60-sec baseline dip in 1×KB, the XX16-coated sensors tips were dipped into the wells containing an anti-XX16 Fab at varying concentrations. Binding interactions were monitored over a 600-sec association period and followed by a 600-sec dissociation period in new wells containing fresh 1×KB. All binding data were referenced using a parallel buffer blank subtraction yielding sensorgrams for association and dissociation phases.

Data processing and analysis including kₒₙ, k_{off}, and K_{D} determination were performed using the Octet® Systems Software (FortéBio). Sensorgrams were fit globally by applying a simple 1:1 Langmuir binding model and the dissociation constants (K_{D}) were calculated from kₒₙ and k_{off} values.

Table 26 shows the dissociation constants as well as on- and off-rates for 9 anti-XX16 Fabs determined by Octet measurements, showing that high affinity anti-XX16 antibodies can be generated by the method described in Example 21 with K_{D} values ranging from 3 to 30 nM.

TABLE 26
Summary of Octet Binding Data for Anti-XX16 Antibodies

| | Average K_{D} | K_{D} error | K_{off} | Kₒₙ |
|---|---|---|---|---|
| Anti-XX16 | [nM] | [nM] | (10⁻⁴/s) | (10⁴/Ms) |
| AA01 | 3.34 | 0.03 | 1.19 | 3.55 |
| AA02 | 8.55 | 0.04 | 3.69 | 4.31 |
| AA03 | 6.29 | 0.03 | 3.70 | 5.89 |
| AA04 | 9.22 | 0.06 | 3.19 | 3.46 |
| AA05 | 3.08 | 0.05 | 0.88 | 2.84 |
| AA06 | 2.96 | 0.03 | 3.34 | 11.6 |
| AA07 | 2.66 | 0.05 | 0.53 | 2.00 |
| AA08 | 33.9 | 0.2 | 13.00 | 3.26 |
| AA09 | 15.7 | 0.2 | 4.15 | 2.65 |

### Example 23: Reversal of XX16-Dependent Activation of hNPR1 In Vitro

The effects of anti-XX16 Fabs on the activity of XX16 were tested in a cGMP homogeneous time-resolved fluorescence (HTRF) assay using CHO-KI cells stably expressing human NPR1.

cGMP HTRF Assay:

CHO-KI cells stably expressing human NPR1 were maintained in Kaighn's modification of Ham's F-12 medium (F-12K) (ATCC), 10% (v/v) FBS (Hyclone, Logan, UT USA), and 400 ng/μl of geneticin (Invitrogen, Carlsbad, CA USA) at 37° C. and 5% CO₂. For measuring cGMP production, the cells were harvested using assay complete detachment medium (DiscoverX) and plated in assay complete cell plating reagent (DiscoverX) at a density of 2000 cells per well into a 384 well white walled microplate (Corning Life Sciences, Corning, NY USA).

Following an 18 hour incubation at 37° C. and 5% CO₂, the plating medium was removed and replaced with 10 μl per well of PBS containing 0.1% (w/v) BSA (Sigma) and 1 mM IBMX (Calbiochem). The anti-XX16 Fabs were tested plus and minus 10 nM final concentration of XX16. For the wells containing XX16, 20 nM of XX16 was added into the aforementioned PBS buffer. The anti-XX16 Fabs were then serially diluted in PBS containing 0.1% (w/v) BSA before adding 10 μl per well to the assay plate and incubating for 3 hours at 37° C. and 5% CO₂.

At the end of the incubation, cGMP production was detected using an HTRF assay (CisBio, Bedford, MA, USA) following the manufacturer's instructions. In brief, 10 μl of cryptate working solution was added to each well to lyse the cells followed by 10 ul of d2 working solution. Negative control wells received 10 μl of PBS containing 0.1% (w/v) BSA instead of the d2 working solution. In addition, a 24 point cGMP standard curve was added to each plate with a top concentration of 1 μM serially diluted (1:1.5 dilutions) down to 133 μM with the last point as a blank. Plates were then sealed and incubated at room temperature for 1 hour before reading on a PerkinElmer Envision instrument using an HTRF readout [Ex 320 nm and dual emission Em 615/665 nm].

cGMP determination from HTRF. Formulas used for determination of cGMP generated in the assay are shown below. The Ratio A/B, where A=the fluorescence emission signal from the LANCE Eu/Cy5 channel (665 nm) and B=the fluorescence emission signal LANCE Eu/Cy5 channel (615 nm), was used for the raw data analysis. The Ratio_{neg} is a negative control data point calculated from a well to which only buffer (no agonist or other test article) has been added. A new value, Delta F was then calculated for each Ratio A/B measurement. The concentration of cGMP in a given well was interpolated from the cGMP standard curve that was independently run as a part of every experiment. The standard curve fits a series of known concentrations of cGMP on the X-axis to their corresponding experimentally determined Delta F values on the Y-axis.

Formulas used for data analysis:

$$1. Ratio = \frac{{A}_{665 nm}}{{B}_{620 nm}} \times {10}^{4}$$

$$2. Mean Ratio = \frac{\sum ratios}{2}$$

$$3. CV \frac{Std deviation}{Mean Ratio} \times 100$$

$$4. Delta F \frac{Calibrator or sample Ratio - {Ratio}_{neg}}{{Ratio}_{neg}}$$

$$( {Ratio}_{neg} = negative control )$$

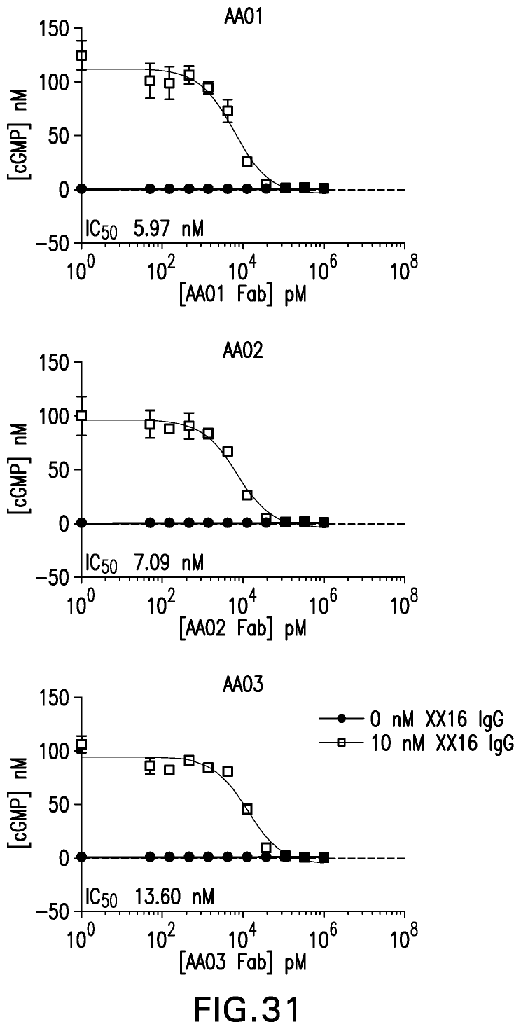

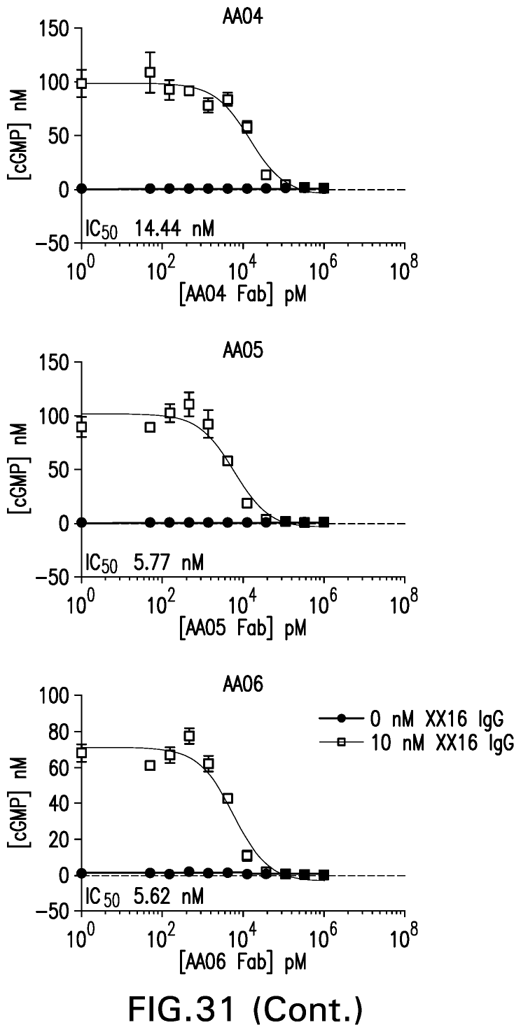

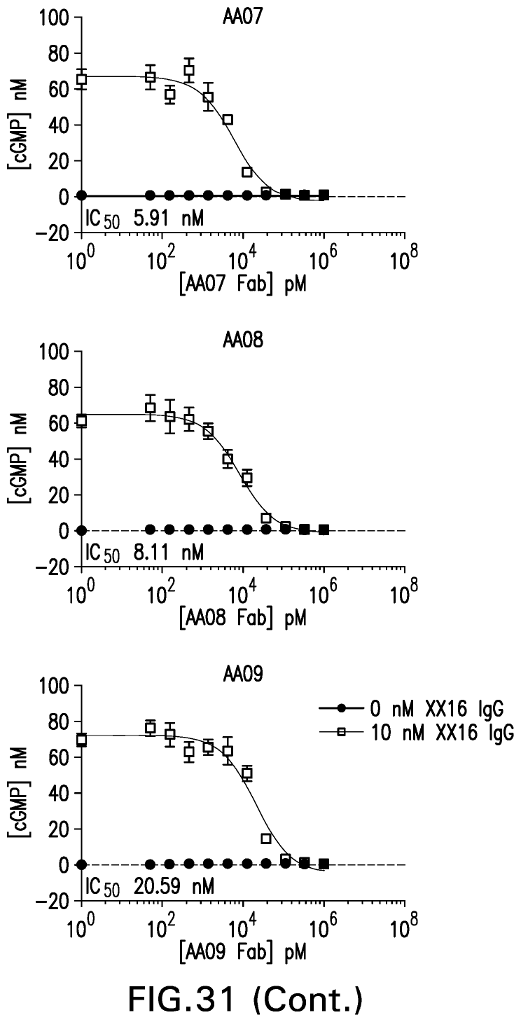

Anti-XX16 Fabs are potent inhibitors of XX16 in the cGMP HTRF assay. To determine if anti-XX16 Fabs can block the activation of NPR1 signaling by XX16, cGMP HTRF assays were performed with mixtures of XX16 and nine different anti-XX16 Fabs. The concentration of XX16 was kept constant at 10 nM, which led to a robust generation of 60-100 nM cGMP in the HTRF assay comparable to the NPR1 ligand ANP at this concentration. When anti-XX16 Fabs were added at increasing concentrations to XX16, the effect of XX16 on cGMP production was dose-dependently reduced. IC₅₀ values of 6 to 21 nM were calculated from the dose response curves, demonstrating that anti-XX16 Fabs described here are potent inhibitors of XX16. Dose response curves and corresponding IC₅₀ values are shown in FIG. 31.

### Example 24: Reversal of XX16-Dependent Activation of hNPR1 In Vivo

To test if an anti-XX16 antibody can reverse the activity of XX16 in vivo, the effect of the anti-idiotype antibody AA07 on XX16-dependent plasma cGMP generation was evaluated in rats.

In Vivo Studies with Sprague Dawley (SD) Rats

Jugular vein-cannulated male SD rats were purchased from Charles River (Charles River Laboratories, Inc.). Animals were acclimated for a minimum of 3 days before being used in an experiment. They were housed on a 12-hour light/dark cycle and provided food and water ad libitum.

Conscious SD rats with pre-cannulated jugular veins were intravenously injected with XX16 formulated at 1 mg/ml in PBS at a dose of 1 mg/kg or with vehicle (PBS). After 24 h XX16 treated animals and vehicle treated animals were intravenously injected with AA07 Fab or AA07 IgG formulated at 3 mg/ml in PBS at a dose of 3 mg/kg. Blood samples were collected via tail snip at the times indicated and mixed with EDTA to prevent coagulation. Plasma was collected and stored in a −80° C. freezer for determination of plasma cGMP levels.

Plasma cGMP Measurements.

An LC-MS/MS detection method for cGMP adopted from Oeckl and Ferger (2012) and Zhang et al. (2009) was used to determine cGMP plasma levels. All plasma samples were equilibrated to ambient room temperature before use. Plasma samples (30 μL) and 120 μL of 40 nmol/L ¹⁵N₂, ¹³C cGMP as internal standard (Toronto Research Chemicals, Toronto, Canada) in methanol were added to a 96-well plate and mixed. After the samples were mixed on a plate shaker at 500 rpm for 5 min, the mixture was centrifuged at 4000 rpm for 10 min at room temperature, and then 100 μL of the supernatants were transferred into a clean 96-well assay plate. Detection and analysis were performed using an LC-MS/MS system (LC20AD/XR (Shimadzu, Kyoto, Japan), QTrap 5500 (Sciex, Framingham, MA) and Shimadzu AC autosampler)). An aliquot (20 μL) of the extract was injected onto a Waters Acquityl UPLC HSS T3, 1.8 m, 3×30 mm reversed-phase column heated to 40° C. The gradient for elution was comprised of 0.1% (v/v) formic acid in water (solvent A) and 0.1% (v/v) formic acid in acetonitrile (solvent B) in the two reservoirs. The initial conditions were 99% solvent A and 1% solvent B at 0.4 mL/minute flow rate for 0.5 min, and a linear gradient was performed with solvent B increasing from 1% to 70% within 2.5 min. Further linear ramping up to 90% of solvent B was performed within 0.5 min. This condition was maintained for 1 min to remove late-eluting substances from the column, followed by returning the system to its initial conditions with a 1.5 min equilibration period. The total run time including column wash and equilibration was 5.5 min. The operating mass parameters were as follows: nebulizer gas, 40 psi; heater gas, 50 psi; curtain gas, 20 psi; temperature, 500° C.; ion spray voltage, 5500 V. The retention time for both cGMP and ¹⁵N₂, ¹³C cGMP was 1.9 min. Quantification of cGMP and ¹⁵N₂, ¹³C cGMP was optimized in negative ionization mode: m/z at 346.28→152.0, declustering potential at 70 V, collision energy at 28 eV for cGMP; m/z at 349.3→155.1, declustering potential at 70 V, collision energy at 28 eV for ¹⁵N, ¹³C cGMP (IS).

Anti-XX16 Antibody AA07 Reverses the Effects of XX16 on cGMP in Rats.

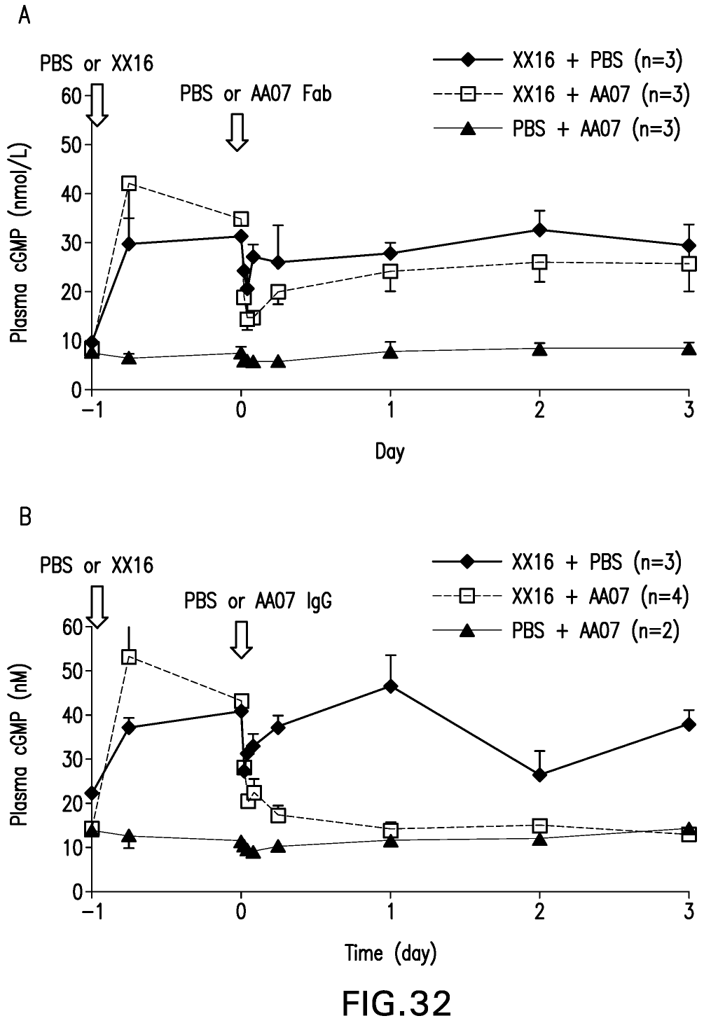

Treating SD rats with XX16 at 1 mg/kg i.v. led to a robust and sustained increase in plasma cGMP levels. FIG. 32, part (A) demonstrates that cGMP plasma levels in SD rats increased 3-4 fold after a single 1 mg/mg i.v. dose of XX16 and were temporarily reduced to close to baseline by anti-XX16 AA07 Fab dosed at 3 mg/ml i.v. FIG. 32, part (B) demonstrates that cGMP plasma levels in SD rats increased about 3 fold after a single 1 mg/mg i.v. dose of XX16 and were reduced to baseline levels through the end of the study (day 3) by anti-XX16 AA07 IgG dosed at 3 mg/ml i.v.

Administration of the anti-XX16 antibody AA07 Fab temporarily decreased cGMP levels close to baseline, but within approximately 8 hours cGMP levels were close the levels observed in animals only treated with XX16. Administration of AA07 IgG also reduced cGMP levels close to baseline, but cGMP levels did not rebound as observed in the AA07 Fab-treated group. Consequently, both anti-XX16 antibodies in Fab and IgG format can reverse the effects of XX16 on cGMP in rats, with the IgG format having a more sustained effect.

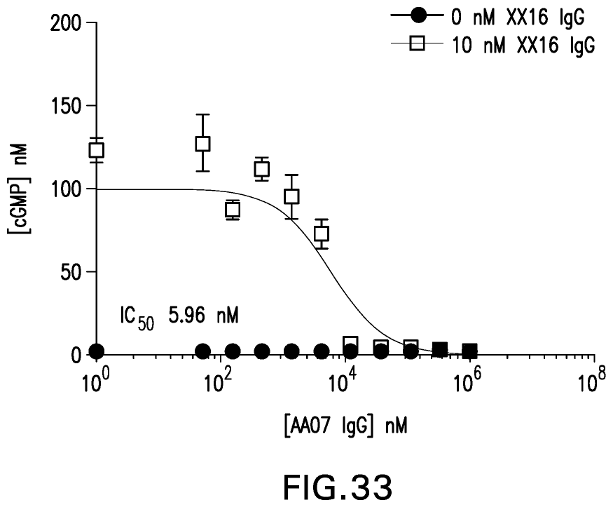

In the cGMP in vitro assay AA07 IgG had similar potency to the AA07 Fab in reversing the effect of XX16 on cGMP generation (FIG. 33).

1. A binding agent which specifically binds an anti-NPR1 target antibody or antigen binding fragment; wherein the target antibody or antigen binding fragment comprises three heavy chain complementarity determining regions (HCDR1, HCDR2, and HCDR3) and three light chain complementarity determining regions (LCDR1, LCDR2, and LCDR3) selected from:
(a) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 134 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 135 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 134 (LCDR3);
(b) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 126 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 126 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 127 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 126 (LCDR3);
(c) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 145 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 145 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 146 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 145 (LCDR3);
(d) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 172 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 172 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 173 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 172 (LCDR3);
(e) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 178 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 178 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 179 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 178 (LCDR3);
(f) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 184 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 184 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 185 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 184 (LCDR3);
(g) (I) SEQ ID NO: 4 (HCDR1), SEQ ID NO: 100 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 100 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 8 (HCDR1), SEQ ID NO: 101 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 10 (HCDR1), SEQ ID NO: 102 (HCDR2), SEQ ID NO: 12 (HCDR3), SEQ ID NO: 23 (LCDR1), SEQ ID NO: 21 (LCDR2), and SEQ ID NO: 19 (LCDR3);
(h) (I) SEQ ID NO: 112 (HCDR1), SEQ ID NO: 100 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 100 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 113 (HCDR1), SEQ ID NO: 101 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 114 (HCDR1), SEQ ID NO: 102 (HCDR2), SEQ ID NO: 12 (HCDR3), SEQ ID NO: 23 (LCDR1), SEQ ID NO: 21 (LCDR2), and SEQ ID NO: 19 (LCDR3);
(i) (I) SEQ ID NO: 28 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 43 (LCDR3); (II) SEQ ID NO: 31 (HCDR1), SEQ ID NO: 119 (HCDR2), SEQ ID NO: 30 (HCDR3), SEQ ID NO: 41 (LCDR1), SEQ ID NO: 42 (LCDR2), and SEQ ID NO: 43 (LCDR3); (III) SEQ ID NO: 32 (HCDR1), SEQ ID NO: 120 (HCDR2), SEQ ID NO: 30 (HCDR3), SEQ ID NO: 44 (LCDR1), SEQ ID NO: 45 (LCDR2), and SEQ ID NO: 46 (LCDR3); or (IV) SEQ ID NO: 34 (HCDR1), SEQ ID NO: 121 (HCDR2), SEQ ID NO: 36 (HCDR3), SEQ ID NO: 47 (LCDR1), SEQ ID NO: 45 (LCDR2), and SEQ ID NO: 43 (LCDR3);
(j) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 126 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 126 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 127 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 126 (LCDR3);
(k) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 134 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 134 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 135 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 134 (LCDR3);
(l) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 145 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 145 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 146 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 145 (LCDR3);
(m) (I) SEQ ID NO: 4 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 8 (HCDR1), SEQ ID NO: 152 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 10 (HCDR1), SEQ ID NO: 153 (HCDR2), SEQ ID NO: 12 (HCDR3), SEQ ID NO: 23 (LCDR1), SEQ ID NO: 21 (LCDR2), and SEQ ID NO: 19 (LCDR3);
(n) (I) SEQ ID NO: 112 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 113 (HCDR1), SEQ ID NO: 152 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 114 (HCDR1), SEQ ID NO: 153 (HCDR2), SEQ ID NO: 12 (HCDR3), SEQ ID NO: 23 (LCDR1), SEQ ID NO: 21 (LCDR2), and SEQ ID NO: 19 (LCDR3);
(o) (I) SEQ ID NO: 165 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 151 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 166 (HCDR1), SEQ ID NO: 152 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 167 (HCDR1), SEQ ID NO: 153 (HCDR2), SEQ ID NO: 12 (HCDR3), SEQ ID NO: 23 (LCDR1), SEQ ID NO: 21 (LCDR2), and SEQ ID NO: 19 (LCDR3);
(p) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 172 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 172 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 173 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 172 (LCDR3);
(q) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 178 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 178 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 179 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 178 (LCDR3);
(r) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 184 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 184 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 185 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 184 (LCDR3);
(s) (I) SEQ ID NO: 28 (HCDR1), SEQ ID NO: 190 (HCDR2), SEQ ID NO: 30 (HCDR3), SEQ ID NO: 41 (LCDR1), SEQ ID NO: 42 (LCDR2), and SEQ ID NO: 134 (LCDR3); (II) SEQ ID NO: 31 (HCDR1), SEQ ID NO: 190 (HCDR2), SEQ ID NO: 30 (HCDR3), SEQ ID NO: 41 (LCDR1), SEQ ID NO: 42 (LCDR2), and SEQ ID NO: 134 (LCDR3); (III) SEQ ID NO: 32 (HCDR1), SEQ ID NO: 191 (HCDR2), SEQ ID NO: 30 (HCDR3), SEQ ID NO: 44 (LCDR1), SEQ ID NO: 45 (LCDR2), and SEQ ID NO: 135 (LCDR3); or (IV) SEQ ID NO: 34 (HCDR1), SEQ ID NO: 192 (HCDR2), SEQ ID NO: 36 (HCDR3), SEQ ID NO: 47 (LCDR1), SEQ ID NO: 45 (LCDR2), and SEQ ID NO: 134 (LCDR3);
(t) (I) SEQ ID NO: 28 (HCDR1), SEQ ID NO: 190 (HCDR2), SEQ ID NO: 30 (HCDR3), SEQ ID NO: 41 (LCDR1), SEQ ID NO: 42 (LCDR2), and SEQ ID NO: 172 (LCDR3); (II) SEQ ID NO: 31 (HCDR1), SEQ ID NO: 190 (HCDR2), SEQ ID NO: 30 (HCDR3), SEQ ID NO: 41 (LCDR1), SEQ ID NO: 42 (LCDR2), and SEQ ID NO: 172 (LCDR3); (III) SEQ ID NO: 32 (HCDR1), SEQ ID NO: 191 (HCDR2), SEQ ID NO: 30 (HCDR3), SEQ ID NO: 44 (LCDR1), SEQ ID NO: 45 (LCDR2), and SEQ ID NO: 173 (LCDR3); or (IV) SEQ ID NO: 34 (HCDR1), SEQ ID NO: 192 (HCDR2), SEQ ID NO: 36 (HCDR3), SEQ ID NO: 47 (LCDR1), SEQ ID NO: 45 (LCDR2), and SEQ ID NO: 172 (LCDR3);
(u) (I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 19 (LCDR3);
(v) (I) SEQ ID NO: 28 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 43 (LCDR3); (II) SEQ ID NO: 31 (HCDR1), SEQ ID NO: 29 (HCDR2), SEQ ID NO: 30 (HCDR3), SEQ ID NO: 41 (LCDR1), SEQ ID NO: 42 (LCDR2), and SEQ ID NO: 43 (LCDR3); (III) SEQ ID NO: 32 (HCDR1), SEQ ID NO: 33 (HCDR2), SEQ ID NO: 30 (HCDR3), SEQ ID NO: 44 (LCDR1), SEQ ID NO: 45 (LCDR2), and SEQ ID NO: 46 (LCDR3); or (IV) SEQ ID NO: 34 (HCDR1), SEQ ID NO: 35 (HCDR2), SEQ ID NO: 36 (HCDR3), SEQ ID NO: 47 (LCDR1), SEQ ID NO: 45 (LCDR2), and SEQ ID NO: 43 (LCDR3);
(w) (I) SEQ ID NO: 367 (HCDR1), SEQ ID NO: 368 (HCDR2), SEQ ID NO: 228 (HCDR3), SEQ ID NO: 237 (LCDR1), SEQ ID NO: 238 (LCDR2), and SEQ ID NO: 239 (LCDR3); (II) SEQ ID NO: 369 (HCDR1), SEQ ID NO: 368 (HCDR2), SEQ ID NO: 228 (HCDR3), SEQ ID NO: 237 (LCDR1), SEQ ID NO: 238 (LCDR2), and SEQ ID NO: 239 (LCDR3); (III) SEQ ID NO: 370 (HCDR1), SEQ ID NO: 371 (HCDR2), SEQ ID NO: 228 (HCDR3), SEQ ID NO: 240 (LCDR1), SEQ ID NO: 241 (LCDR2), and SEQ ID NO: 242 (LCDR3); or (IV) SEQ ID NO: 372 (HCDR1), SEQ ID NO: 373 (HCDR2), SEQ ID NO: 232 (HCDR3), SEQ ID NO: 243 (LCDR1), SEQ ID NO: 241 (LCDR2), and SEQ ID NO: 239 (LCDR3);
(x) (I) SEQ ID NO: 378 (HCDR1), SEQ ID NO: 379 (HCDR2), SEQ ID NO: 228 (HCDR3), SEQ ID NO: 237 (LCDR1), SEQ ID NO: 238 (LCDR2), and SEQ ID NO: 239 (LCDR3); (II) SEQ ID NO: 380 (HCDR1), SEQ ID NO: 379 (HCDR2), SEQ ID NO: 228 (HCDR3), SEQ ID NO: 237 (LCDR1), SEQ ID NO: 238 (LCDR2), and SEQ ID NO: 239 (LCDR3); (III) SEQ ID NO: 381 (HCDR1), SEQ ID NO: 382 (HCDR2), SEQ ID NO: 228 (HCDR3), SEQ ID NO: 240 (LCDR1), SEQ ID NO: 241 (LCDR2), and SEQ ID NO: 242 (LCDR3); or (IV) SEQ ID NO: 383 (HCDR1), SEQ ID NO: 384 (HCDR2), SEQ ID NO: 232 (HCDR3), SEQ ID NO: 243 (LCDR1), SEQ ID NO: 241 (LCDR2), and SEQ ID NO: 239 (LCDR3);
(y) (I) SEQ ID NO: 226 (HCDR1), SEQ ID NO: 227 (HCDR2), SEQ ID NO: 228 (HCDR3), SEQ ID NO: 237 (LCDR1), SEQ ID NO: 238 (LCDR2), and SEQ ID NO: 239 (LCDR3); (II) SEQ ID NO: 229 (HCDR1), SEQ ID NO: 227 (HCDR2), SEQ ID NO: 228 (HCDR3), SEQ ID NO: 237 (LCDR1), SEQ ID NO: 238 (LCDR2), and SEQ ID NO: 239 (LCDR3); (III) SEQ ID NO: 32 (HCDR1), SEQ ID NO: 230 (HCDR2), SEQ ID NO: 228 (HCDR3), SEQ ID NO: 240 (LCDR1), SEQ ID NO: 241 (LCDR2), and SEQ ID NO: 242 (LCDR3); or (IV) SEQ ID NO: 34 (HCDR1), SEQ ID NO: 231 (HCDR2), SEQ ID NO: 232 (HCDR3), SEQ ID NO: 243 (LCDR1), SEQ ID NO: 241 (LCDR2), and SEQ ID NO: 239 (LCDR3);
(z) (I) SEQ ID NO: 270 (HCDR1), SEQ ID NO: 271 (HCDR2), SEQ ID NO: 272 (HCDR3), SEQ ID NO: 282 (LCDR1), SEQ ID NO: 261 (LCDR2), and SEQ ID NO: 283 (LCDR3); (II) SEQ ID NO: 273 (HCDR1), SEQ ID NO: 271 (HCDR2), SEQ ID NO: 272 (HCDR3), SEQ ID NO: 282 (LCDR1), SEQ ID NO: 261 (LCDR2), and SEQ ID NO: 283 (LCDR3); (III) SEQ ID NO: 32 (HCDR1), SEQ ID NO: 274 (HCDR2), SEQ ID NO: 272 (HCDR3), SEQ ID NO: 284 (LCDR1), SEQ ID NO: 241 (LCDR2), and SEQ ID NO: 285 (LCDR3); or (IV) SEQ ID NO: 275 (HCDR1), SEQ ID NO: 276 (HCDR2), SEQ ID NO: 277 (HCDR3), SEQ ID NO: 286 (LCDR1), SEQ ID NO: 241 (LCDR2), and SEQ ID NO: 283 (LCDR3);
(aa) (I) SEQ ID NO: 291 (HCDR1), SEQ ID NO: 292 (HCDR2), SEQ ID NO: 293 (HCDR3), SEQ ID NO: 237 (LCDR1), SEQ ID NO: 238 (LCDR2), and SEQ ID NO: 304 (LCDR3); (II) SEQ ID NO: 294 (HCDR1), SEQ ID NO: 292 (HCDR2), SEQ ID NO: 293 (HCDR3), SEQ ID NO: 237 (LCDR1), SEQ ID NO: 238 (LCDR2), and SEQ ID NO: 304 (LCDR3); (III) SEQ ID NO: 295 (HCDR1), SEQ ID NO: 296 (HCDR2), SEQ ID NO: 293 (HCDR3), SEQ ID NO: 240 (LCDR1), SEQ ID NO: 241 (LCDR2), and SEQ ID NO: 305 (LCDR3); or (IV) SEQ ID NO: 297 (HCDR1), SEQ ID NO: 298 (HCDR2), SEQ ID NO: 299 (HCDR3), SEQ ID NO: 243 (LCDR1), SEQ ID NO: 241 (LCDR2), and SEQ ID NO: 304 (LCDR3);
(bb) (I) SEQ ID NO: 52 (HCDR1), SEQ ID NO: 53 (HCDR2), SEQ ID NO: 54 (HCDR3), SEQ ID NO: 65 (LCDR1), SEQ ID NO: 66 (LCDR2), and SEQ ID NO: 67 (LCDR3); (II) SEQ ID NO: 55 (HCDR1), SEQ ID NO: 53 (HCDR2), SEQ ID NO: 54 (HCDR3), SEQ ID NO: 65 (LCDR1), SEQ ID NO: 66 (LCDR2), and SEQ ID NO: 67 (LCDR3); (III) SEQ ID NO: 56 (HCDR1), SEQ ID NO: 57 (HCDR2), SEQ ID NO: 54 (HCDR3), SEQ ID NO: 68 (LCDR1), SEQ ID NO: 69 (LCDR2), and SEQ ID NO: 70 (LCDR3); or (IV) SEQ ID NO: 58 (HCDR1), SEQ ID NO: 59 (HCDR2), SEQ ID NO: 60 (HCDR3), SEQ ID NO: 71 (LCDR1), SEQ ID NO: 69 (LCDR2), and SEQ ID NO: 67 (LCDR3);
(cc) (I) SEQ ID NO: 76 (HCDR1), SEQ ID NO: 77 (HCDR2), SEQ ID NO: 78 (HCDR3), SEQ ID NO: 89 (LCDR1), SEQ ID NO: 90 (LCDR2), and SEQ ID NO: 91 (LCDR3); (II) SEQ ID NO: 79 (HCDR1), SEQ ID NO: 77 (HCDR2), SEQ ID NO: 78 (HCDR3), SEQ ID NO: 89 (LCDR1), SEQ ID NO: 90 (LCDR2), and SEQ ID NO: 91 (LCDR3); (III) SEQ ID NO: 80 (HCDR1), SEQ ID NO: 81 (HCDR2), SEQ ID NO: 78 (HCDR3), SEQ ID NO: 92 (LCDR1), SEQ ID NO: 93 (LCDR2), and SEQ ID NO: 94 (LCDR3); or (IV) SEQ ID NO: 82 (HCDR1), SEQ ID NO: 83 (HCDR2), SEQ ID NO: 84 (HCDR3), SEQ ID NO: 95 (LCDR1), SEQ ID NO: 93 (LCDR2), and SEQ ID NO: 91 (LCDR3);
(dd) (I) SEQ ID NO: 310 (HCDR1), SEQ ID NO: 311 (HCDR2), SEQ ID NO: 348 (HCDR3), SEQ ID NO: 320 (LCDR1), SEQ ID NO: 354 (LCDR2), and SEQ ID NO: 361 (LCDR3); (II) SEQ ID NO: 229 (HCDR1), SEQ ID NO: 311 (HCDR2), SEQ ID NO: 348 (HCDR3), SEQ ID NO: 320 (LCDR1), SEQ ID NO: 354 (LCDR2), and SEQ ID NO: 361 (LCDR3); (III) SEQ ID NO: 80 (HCDR1), SEQ ID NO: 313 (HCDR2), SEQ ID NO: 348 (HCDR3), SEQ ID NO: 323 (LCDR1), SEQ ID NO: 324 (LCDR2), and SEQ ID NO: 362 (LCDR3); or (IV) SEQ ID NO: 82 (HCDR1), SEQ ID NO: 314 (HCDR2), SEQ ID NO: 349 (HCDR3), SEQ ID NO: 326 (LCDR1), SEQ ID NO: 324 (LCDR2), and SEQ ID NO: 361 (LCDR3);
(ee) (I) SEQ ID NO: 270 (HCDR1), SEQ ID NO: 389 (HCDR2), SEQ ID NO: 331 (HCDR3), SEQ ID NO: 337 (LCDR1), SEQ ID NO: 338 (LCDR2), and SEQ ID NO: 339 (LCDR3); (II) SEQ ID NO: 273 (HCDR1), SEQ ID NO: 389 (HCDR2), SEQ ID NO: 331 (HCDR3), SEQ ID NO: 337 (LCDR1), SEQ ID NO: 338 (LCDR2), and SEQ ID NO: 339 (LCDR3); (III) SEQ ID NO: 32 (HCDR1), SEQ ID NO: 390 (HCDR2), SEQ ID NO: 331 (HCDR3), SEQ ID NO: 340 (LCDR1), SEQ ID NO: 341 (LCDR2), and SEQ ID NO: 342 (LCDR3); or (IV) SEQ ID NO: 275 (HCDR1), SEQ ID NO: 391 (HCDR2), SEQ ID NO: 332 (HCDR3), SEQ ID NO: 343 (LCDR1), SEQ ID NO: 341 (LCDR2), and SEQ ID NO: 339 (LCDR3);
(ff) (I) SEQ ID NO: 407 (HCDR1), SEQ ID NO: 408 (HCDR2), SEQ ID NO: 331 (HCDR3), SEQ ID NO: 337 (LCDR1), SEQ ID NO: 338 (LCDR2), and SEQ ID NO: 339 (LCDR3); (II) SEQ ID NO: 409 (HCDR1), SEQ ID NO: 408 (HCDR2), SEQ ID NO: 331 (HCDR3), SEQ ID NO: 337 (LCDR1), SEQ ID NO: 338 (LCDR2), and SEQ ID NO: 339 (LCDR3); (III) SEQ ID NO: 410 (HCDR1), SEQ ID NO: 411 (HCDR2), SEQ ID NO: 331 (HCDR3), SEQ ID NO: 340 (LCDR1), SEQ ID NO: 341 (LCDR2), and SEQ ID NO: 342 (LCDR3); or (IV) SEQ ID NO: 412 (HCDR1), SEQ ID NO: 413 (HCDR2), SEQ ID NO: 332 (HCDR3), SEQ ID NO: 343 (LCDR1), SEQ ID NO: 341 (LCDR2), and SEQ ID NO: 339 (LCDR3);
(qq) (I) SEQ ID NO: 310 (HCDR1), SEQ ID NO: 311 (HCDR2), SEQ ID NO: 312 (HCDR3), SEQ ID NO: 320 (LCDR1), SEQ ID NO: 321 (LCDR2), and SEQ ID NO: 322 (LCDR3); (II) SEQ ID NO: 229 (HCDR1), SEQ ID NO: 311 (HCDR2), SEQ ID NO: 312 (HCDR3), SEQ ID NO: 320 (LCDR1), SEQ ID NO: 321 (LCDR2), and SEQ ID NO: 322 (LCDR3); (III) SEQ ID NO: 80 (HCDR1), SEQ ID NO: 313 (HCDR2), SEQ ID NO: 312 (HCDR3), SEQ ID NO: 323 (LCDR1), SEQ ID NO: 324 (LCDR2), and SEQ ID NO: 325 (LCDR3); or (IV) SEQ ID NO: 82 (HCDR1), SEQ ID NO: 314 (HCDR2), SEQ ID NO: 315 (HCDR3), SEQ ID NO: 326 (LCDR1), SEQ ID NO: 324 (LCDR2), and SEQ ID NO: 322 (LCDR3);
(hh) (I) SEQ ID NO: 270 (HCDR1), SEQ ID NO: 271 (HCDR2), SEQ ID NO: 331 (HCDR3), SEQ ID NO: 337 (LCDR1), SEQ ID NO: 338 (LCDR2), and SEQ ID NO: 339 (LCDR3); (II) SEQ ID NO: 273 (HCDR1), SEQ ID NO: 271 (HCDR2), SEQ ID NO: 331 (HCDR3), SEQ ID NO: 337 (LCDR1), SEQ ID NO: 338 (LCDR2), and SEQ ID NO: 339 (LCDR3); (III) SEQ ID NO: 32 (HCDR1), SEQ ID NO: 274 (HCDR2), SEQ ID NO: 331 (HCDR3), SEQ ID NO: 340 (LCDR1), SEQ ID NO: 341 (LCDR2), and SEQ ID NO: 342 (LCDR3); or (IV) SEQ ID NO: 275 (HCDR1), SEQ ID NO: 276 (HCDR2), SEQ ID NO: 332 (HCDR3), SEQ ID NO: 343 (LCDR1), SEQ ID NO: 341 (LCDR2), and SEQ ID NO: 339 (LCDR3); or
(ii) (I) SEQ ID NO: 310 (HCDR1), SEQ ID NO: 311 (HCDR2), SEQ ID NO: 348 (HCDR3), SEQ ID NO: 320 (LCDR1), SEQ ID NO: 354 (LCDR2), and SEQ ID NO: 355 (LCDR3); (II) SEQ ID NO: 229 (HCDR1), SEQ ID NO: 311 (HCDR2), SEQ ID NO: 348 (HCDR3), SEQ ID NO: 320 (LCDR1), SEQ ID NO: 354 (LCDR2), and SEQ ID NO: 355 (LCDR3); (III) SEQ ID NO: 80 (HCDR1), SEQ ID NO: 313 (HCDR2), SEQ ID NO: 348 (HCDR3), SEQ ID NO: 323 (LCDR1), SEQ ID NO: 324 (LCDR2), and SEQ ID NO: 356 (LCDR3); or (IV) SEQ ID NO: 82 (HCDR1), SEQ ID NO: 314 (HCDR2), SEQ ID NO: 349 (HCDR3), SEQ ID NO: 326 (LCDR1), SEQ ID NO: 324 (LCDR2), and SEQ ID NO: 355 (LCDR3).

2. (canceled)

3. A binding agent of claim 1, wherein the target antibody or antigen binding fragment comprises:
(a) a heavy chain variable region comprising an amino acid sequence of SEQ ID NO: 201, and a light chain variable region comprising an amino acid sequence of SEQ ID NO: 136;
(b) a heavy chain variable region comprising an amino acid sequence of SEQ ID NO: 122, and a light chain variable region comprising an amino acid sequence of SEQ ID NO: 136;
(c) a heavy chain variable region comprising an amino acid sequence of SEQ ID NO: 201, and a light chain variable region comprising an amino acid sequence of SEQ ID NO: 128;
(d) a heavy chain variable region comprising an amino acid sequence of SEQ ID NO: 122, and a light chain variable region comprising an amino acid sequence of SEQ ID NO: 128;
(e) a heavy chain variable region comprising an amino acid sequence of SEQ ID NO: 201, and a light chain variable region comprising an amino acid sequence of SEQ ID NO: 147;
(f) a heavy chain variable region comprising an amino acid sequence of SEQ ID NO: 122, and a light chain variable region comprising an amino acid sequence of SEQ ID NO: 147;
(g) a heavy chain variable region comprising an amino acid sequence of SEQ ID NO: 201, and a light chain variable region comprising an amino acid sequence of SEQ ID NO: 174;
(h) a heavy chain variable region comprising an amino acid sequence of SEQ ID NO: 122, and a light chain variable region comprising an amino acid sequence of SEQ ID NO: 174;
(i) a heavy chain variable region comprising an amino acid sequence of SEQ ID NO: 201, and a light chain variable region comprising an amino acid sequence of SEQ ID NO: 180;
(j) a heavy chain variable region comprising an amino acid sequence of SEQ ID NO: 122, and a light chain variable region comprising an amino acid sequence of SEQ ID NO: 180;
(k) a heavy chain variable region comprising an amino acid sequence of SEQ ID NO: 201, and a light chain variable region comprising an amino acid sequence of SEQ ID NO: 186;
(l) a heavy chain variable region comprising an amino acid sequence of SEQ ID NO: 122, and a light chain variable region comprising an amino acid sequence of SEQ ID NO: 186;
(m) a heavy chain variable region comprising an amino acid sequence of SEQ ID NO: 103, and a light chain variable region comprising an amino acid sequence of SEQ ID NO: 24;
(n) a heavy chain variable region comprising an amino acid sequence of SEQ ID NO: 115, and a light chain variable region comprising an amino acid sequence of SEQ ID NO: 24;
(o) a heavy chain variable region comprising an amino acid sequence of SEQ ID NO: 122, and a light chain variable region comprising an amino acid sequence of SEQ ID NO: 48;
(p) a heavy chain variable region comprising an amino acid sequence of SEQ ID NO: 37, and a light chain variable region comprising an amino acid sequence of SEQ ID NO: 128;
(q) a heavy chain variable region comprising an amino acid sequence of SEQ ID NO: 37, and a light chain variable region comprising an amino acid sequence of SEQ ID NO: 136;
(r) a heavy chain variable region comprising an amino acid sequence of SEQ ID NO: 37, and a light chain variable region comprising an amino acid sequence of SEQ ID NO: 147;
(s) a heavy chain variable region comprising an amino acid sequence of SEQ ID NO: 154, and a light chain variable region comprising an amino acid sequence of SEQ ID NO: 24;
(t) a heavy chain variable region comprising an amino acid sequence of SEQ ID NO: 161, and a light chain variable region comprising an amino acid sequence of SEQ ID NO: 24;
(u) a heavy chain variable region comprising an amino acid sequence of SEQ ID NO: 168, and a light chain variable region comprising an amino acid sequence of SEQ ID NO: 24;
(v) a heavy chain variable region comprising an amino acid sequence of SEQ ID NO: 37, and a light chain variable region comprising an amino acid sequence of SEQ ID NO: 174;
(w) a heavy chain variable region comprising an amino acid sequence of SEQ ID NO: 37, and a light chain variable region comprising an amino acid sequence of SEQ ID NO: 180;
(x) a heavy chain variable region comprising an amino acid sequence of SEQ ID NO: 37, and a light chain variable region comprising an amino acid sequence of SEQ ID NO: 186;
(y) a heavy chain variable region comprising an amino acid sequence of SEQ ID NO: 193, and a light chain variable region comprising an amino acid sequence of SEQ ID NO: 136;
(z) a heavy chain variable region comprising an amino acid sequence of SEQ ID NO: 193, and a light chain variable region comprising an amino acid sequence of SEQ ID NO: 174;
(aa) a heavy chain variable region comprising an amino acid sequence of SEQ ID NO: 13, and a light chain variable region comprising an amino acid sequence of SEQ ID NO: 24;
(bb) a heavy chain variable region comprising an amino acid sequence of SEQ ID NO: 37, and a light chain variable region comprising an amino acid sequence of SEQ ID NO: 48;
(cc) a heavy chain variable region comprising an amino acid sequence of SEQ ID NO: 374, and a light chain variable region comprising an amino acid sequence of SEQ ID NO: 244;
(dd) a heavy chain variable region comprising an amino acid sequence of SEQ ID NO: 385, and a light chain variable region comprising an amino acid sequence of SEQ ID NO: 244;
(ee) a heavy chain variable region comprising an amino acid sequence of SEQ ID NO: 233, and a light chain variable region comprising an amino acid sequence of SEQ ID NO: 244;
(ff) a heavy chain variable region comprising an amino acid sequence of SEQ ID NO: 278, and a light chain variable region comprising an amino acid sequence of SEQ ID NO: 287;
(gg) a heavy chain variable region comprising an amino acid sequence of SEQ ID NO: 300, and a light chain variable region comprising an amino acid sequence of SEQ ID NO: 306;
(hh) a heavy chain variable region comprising an amino acid sequence of SEQ ID NO: 61, and a light chain variable region comprising an amino acid sequence of SEQ ID NO: 72;
(ii) a heavy chain variable region comprising an amino acid sequence of SEQ ID NO: 85, and a light chain variable region comprising an amino acid sequence of SEQ ID NO: 96;
(jj) a heavy chain variable region comprising an amino acid sequence of SEQ ID NO: 350, and a light chain variable region comprising an amino acid sequence of SEQ ID NO: 363;
(kk) a heavy chain variable region comprising an amino acid sequence of SEQ ID NO: 392, and a light chain variable region comprising an amino acid sequence of SEQ ID NO: 344;
(ll) a heavy chain variable region comprising an amino acid sequence of SEQ ID NO: 414, and a light chain variable region comprising an amino acid sequence of SEQ ID NO: 344;
(mm) a heavy chain variable region comprising an amino acid sequence of SEQ ID NO: 316, and a light chain variable region comprising an amino acid sequence of SEQ ID NO: 327;
(nn) a heavy chain variable region comprising an amino acid sequence of SEQ ID NO: 333, and a light chain variable region comprising an amino acid sequence of SEQ ID NO: 344; or
(oo) a heavy chain variable region comprising an amino acid sequence of SEQ ID NO: 350, and a light chain variable region comprising an amino acid sequence of SEQ ID NO: 357.

4. (canceled)

5. A binding agent of claim 1, wherein the target antibody or antigen binding fragment comprises:
(a) a heavy chain comprising an amino acid sequence of SEQ ID NO: 203, and a light chain comprising an amino acid sequence of SEQ ID NO: 138;
(b) a heavy chain comprising an amino acid sequence of SEQ ID NO: 208, and a light chain comprising an amino acid sequence of SEQ ID NO: 138;
(c) a heavy chain comprising an amino acid sequence of SEQ ID NO: 203, and a light chain comprising an amino acid sequence of SEQ ID NO: 130;
(d) a heavy chain comprising an amino acid sequence of SEQ ID NO: 208, and a light chain comprising an amino acid sequence of SEQ ID NO: 130;
(e) a heavy chain comprising an amino acid sequence of SEQ ID NO: 203, and a light chain comprising an amino acid sequence of SEQ ID NO: 149;
(f) a heavy chain comprising an amino acid sequence of SEQ ID NO: 208, and a light chain comprising an amino acid sequence of SEQ ID NO: 149;
(g) a heavy chain comprising an amino acid sequence of SEQ ID NO: 203, and a light chain comprising an amino acid sequence of SEQ ID NO: 176;
(h) a heavy chain comprising an amino acid sequence of SEQ ID NO: 208, and a light chain comprising an amino acid sequence of SEQ ID NO: 176;
(i) a heavy chain comprising an amino acid sequence of SEQ ID NO: 203, and a light chain comprising an amino acid sequence of SEQ ID NO: 182;
(j) a heavy chain comprising an amino acid sequence of SEQ ID NO: 208, and a light chain comprising an amino acid sequence of SEQ ID NO: 182;
(k) a heavy chain comprising an amino acid sequence of SEQ ID NO: 203, and a light chain comprising an amino acid sequence of SEQ ID NO: 188;
(l) a heavy chain comprising an amino acid sequence of SEQ ID NO: 208, and a light chain comprising an amino acid sequence of SEQ ID NO: 188;
(m) a heavy chain comprising an amino acid sequence of SEQ ID NO: 105, and a light chain comprising an amino acid sequence of SEQ ID NO: 26;
(n) a heavy chain comprising an amino acid sequence of SEQ ID NO: 108, and a light chain comprising an amino acid sequence of SEQ ID NO: 26;
(o) a heavy chain comprising an amino acid sequence of SEQ ID NO: 117, and a light chain comprising an amino acid sequence of SEQ ID NO: 26;
(p) a heavy chain comprising an amino acid sequence of SEQ ID NO: 124, and a light chain comprising an amino acid sequence of SEQ ID NO: 50;
(q) a heavy chain comprising an amino acid sequence of SEQ ID NO: 39, and a light chain comprising an amino acid sequence of SEQ ID NO: 130;
(r) a heavy chain comprising an amino acid sequence of SEQ ID NO: 39, and a light chain comprising an amino acid sequence of SEQ ID NO: 138;
(s) a heavy chain comprising an amino acid sequence of SEQ ID NO: 141, and a light chain comprising an amino acid sequence of SEQ ID NO: 138;
(t) a heavy chain comprising an amino acid sequence of SEQ ID NO: 39, and a light chain comprising an amino acid sequence of SEQ ID NO: 149;
(u) a heavy chain comprising an amino acid sequence of SEQ ID NO: 156, and a light chain comprising an amino acid sequence of SEQ ID NO: 26;
(v) a heavy chain comprising an amino acid sequence of SEQ ID NO: 159, and a light chain comprising an amino acid sequence of SEQ ID NO: 26;
(w) a heavy chain comprising an amino acid sequence of SEQ ID NO: 163, and a light chain comprising an amino acid sequence of SEQ ID NO: 26;
(x) a heavy chain comprising an amino acid sequence of SEQ ID NO: 170, and a light chain comprising an amino acid sequence of SEQ ID NO: 26;
(y) a heavy chain comprising an amino acid sequence of SEQ ID NO: 39, and a light chain comprising an amino acid sequence of SEQ ID NO: 176;
(z) a heavy chain comprising an amino acid sequence of SEQ ID NO: 39, and a light chain comprising an amino acid sequence of SEQ ID NO: 182;
(aa) a heavy chain comprising an amino acid sequence of SEQ ID NO: 39, and a light chain comprising an amino acid sequence of SEQ ID NO: 188;
(bb) a heavy chain comprising an amino acid sequence of SEQ ID NO: 195, and a light chain comprising an amino acid sequence of SEQ ID NO: 138;
(cc) a heavy chain comprising an amino acid sequence of SEQ ID NO: 195, and a light chain comprising an amino acid sequence of SEQ ID NO: 176;
(dd) a heavy chain comprising an amino acid sequence of SEQ ID NO: 15, and a light chain comprising an amino acid sequence of SEQ ID NO: 26;
(ee) a heavy chain comprising an amino acid sequence of SEQ ID NO: 39, and a light chain comprising an amino acid sequence of SEQ ID NO: 50;
(ff) a heavy chain comprising an amino acid sequence of SEQ ID NO: 376, and a light chain comprising an amino acid sequence of SEQ ID NO: 246;
(gg) a heavy chain comprising an amino acid sequence of SEQ ID NO: 387, and a light chain comprising an amino acid sequence of SEQ ID NO: 246;
(hh) a heavy chain comprising an amino acid sequence of SEQ ID NO: 235, and a light chain comprising an amino acid sequence of SEQ ID NO: 246;
(ii) a heavy chain comprising an amino acid sequence of SEQ ID NO: 280, and a light chain comprising an amino acid sequence of SEQ ID NO: 289;
(jj) a heavy chain comprising an amino acid sequence of SEQ ID NO: 302, and a light chain comprising an amino acid sequence of SEQ ID NO: 308;
(kk) a heavy chain comprising an amino acid sequence of SEQ ID NO: 63, and a light chain comprising an amino acid sequence of SEQ ID NO: 74;
(ll) a heavy chain comprising an amino acid sequence of SEQ ID NO: 87, and a light chain comprising an amino acid sequence of SEQ ID NO: 98;
(mm) a heavy chain comprising an amino acid sequence of SEQ ID NO: 352, and a light chain comprising an amino acid sequence of SEQ ID NO: 365;
(nn) a heavy chain comprising an amino acid sequence of SEQ ID NO: 394, and a light chain comprising an amino acid sequence of SEQ ID NO: 346;
(oo) a heavy chain comprising an amino acid sequence of SEQ ID NO: 416, and a light chain comprising an amino acid sequence of SEQ ID NO: 346;
(pp) a heavy chain comprising an amino acid sequence of SEQ ID NO: 318, and a light chain comprising an amino acid sequence of SEQ ID NO: 329;
(qq) a heavy chain comprising an amino acid sequence of SEQ ID NO: 335, and a light chain comprising an amino acid sequence of SEQ ID NO: 346; or
(rr) a heavy chain comprising an amino acid sequence of SEQ ID NO: 352, and a light chain comprising an amino acid sequence of SEQ ID NO: 359.

6. (canceled)

7. The binding agent of claim 1, wherein the binding agent is an antibody or an antigen-binding fragment thereof.

8. The binding agent of claim 1, wherein the binding agent is a monoclonal human antibody.

9. The binding agent of claim 1, wherein the binding agent is an antigen binding fragment selected from the group consisting of a Fab, Fab′, F(ab′)₂, Fv, single domain antibody (dAb), and a single chain variable fragment (scFv).

10.-15. (canceled)

16. A pharmaceutical composition comprising an effective amount of the binding agent of claim 1.

17. A method for reversing the hypotensive effect of an anti-NPR1 target antibody or antigen-binding fragment thereof in a subject being treated with the anti-NPR1 target antibody or antigen-binding fragment thereof, comprising administering an effective amount of the binding agent of claim 1 to a subject in need thereof.

18. The method of claim 17, wherein the anti-NPR1 target antibody or antigen-binding fragment thereof competes with, or binds to the same epitope as, a reference anti-NPR1 target antibody or antigen-binding fragment thereof comprising a heavy chain comprising the amino acid sequence of SEQ ID NO: 208 and a light chain comprising the amino acid sequence of SEQ ID NO: 138.

19. The method of claim 17, wherein the method further comprises additionally administering one of the following to the subject: (i) fluid infusion; and/or (ii) one or more vasopressors; optionally wherein the fluid infusion is a crystalloid IV fluid infusion or a saline infusion; optionally wherein the one or more vasopressors is selected from phenylephrine, norepinephrine, epinephrine, and vasopressin.

20. The method of claim 17, wherein the method additionally comprises halting treatment with an additional therapeutic agent; optionally wherein the additional therapeutic agent is an anti-hypertensive agent, a beta blocker, and/or a diuretic.

21. The method of claim 17, wherein the subject has or is at risk of developing a cardiovascular disorder; optionally wherein the cardiovascular disorder is selected from: hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, and myocardial infarction (MI).

22. The method of claim 17, wherein the subject has or is at risk of developing heart failure, hypertrophic cardiomyopathy (HCM), hypertension, preeclampsia, asthma, glaucoma, a kidney disorder, and/or cytokine release syndrome; optionally wherein the heart failure is selected from a heart failure with reduced ejection fraction (HFrEF), heart failure with preserved ejection fraction (HFpEF), heart failure after acute myocardial infarct, or acute decompensated heart failure; optionally wherein the hypertension is selected from resistant hypertension, hypertensive heart disease, pulmonary hypertension, pulmonary arterial hypertension, and isolated systolic hypertension.

23. An anti-idiotype antibody that specifically binds to an anti-NPR1 antibody or antigen binding fragment thereof wherein the anti-NPR1 antibody or antigen binding fragment thereof is XX16 or comprises the heavy chain variable region CDRs and light chain variable region CDRs of XX16.

24.-28. (canceled)

29. A method of managing hypotension risk or low blood pressure in subject treated with or administered an anti-NPR1 antibody or antigen binding fragment thereof, comprising the step of administering to the subject in need thereof the anti-idiotype antibody of claim 23, wherein the anti-idiotype blocks the anti-NPR1 antibody or antigen binding fragment thereof from binding to NPR1, optionally wherein the anti-idiotype antibody or antigen binding fragment thereof reverses the hypotensive effects of the anti-NPR1 antibody or low blood pressure of the subject.

30. A method of reversing and/or inhibiting the generation of cGMP caused by an anti-NPR1 antibody or antigen binding fragment thereof in a subject in need of such treatment, comprising the step of administering to the subject in need thereof the anti-idiotype antibody of claim 23, wherein the anti-idiotype blocks the anti-NPR1 antibody or antigen binding fragment thereof from binding to NPR1.

31. The binding agent of claim 1, wherein the binding agent blocks the anti-NPR1 antibody or antigen binding fragment thereof from binding to NPR1.

32. A method of treating a disorder or disease associated with administration of an anti-NPR1 target antibody or antigen binding fragment in a subject in need of such treatment, comprising administering an effective amount of the binding agent of claim 1 to a subject in need thereof.

33. The method of claim 32, wherein the binding agent reverses and/or inhibits the agonism of the NPR1 pathway or the generation of cGMP caused by the target antibody or antigen binding fragment thereof in a subject in need of such treatment.

34. The method of claim 32, wherein the disease or disorder is hypotension.


SEQUENCE LISTING (473 sequences)

SEQ ID NO: 1 - protein, 1061 aa, Homo sapiens
MPGPRRPAGSRLRLLLLLLLPPLLLLLRGSHAGNLTVAVVLPLANTSYPWSWARVGPAVELALAQVKARPDLLPGWTVRTVLGSSENALGVCSDTAAPLAAVDLKWEHNPAVFLGPGCVYAAAPVGRFTAHWRVPLLTAGAPALGFGVKDEYALTTRAGPSYAKLGDFVAALHRRLGWERQALMLYAYRPGDEEHCFFLVEGLFMRVRDRLNITVDHLEFAEDDLSHYTRLLRTMPRKGRVIYICSSPDAFRTLMLLALEAGLCGEDYVFFHLDIFGQSLQGGQGPAPRRPWERGDGQDVSARQAFQAAKIITYKDPDNPEYLEFLKQLKHLAYEQFNFTMEDGLVNTIPASFHDGLLLYIQAVTETLAHGGTVTDGENITQRMWNRSFQGVTGYLKIDSSGDRETDFSLWDMDPENGAFRVVLNYNGTSQELVAVSGRKLNWPLGYPPPDIPKCGFDNEDPACNQDHLSTLEVLALVGSLSLLGILIVSFFIYRKMQLEKELASELWRVRWEDVEPSSLERHLRSAGSRLTLSGRGSNYGSLLTTEGQFQVFAKTAYYKGNLVAVKRVNRKRIELTRKVLFELKHMRDVQNEHLTRFVGACTDPPNICILTEYCPRGSLQDILENESITLDWMFRYSLTNDIVKGMLFLHNGAICSHGNLKSSNCVVDGRFVLKITDYGLESFRDLDPEQGHTVYAKKLWTAPELLRMASPPVRGSQAGDVYSFGIILQEIALRSGVFHVEGLDLSPKEIIERVTRGEQPPFRPSLALQSHLEELGLLMQRCWAEDPQERPPFQQIRLTLRKFNRENSSNILDNLLSRMEQYANNLEELVEERTQAYLEEKRKAEALLYQILPHSVAEQLKRGETVQAEAFDSVTIYFSDIVGFTALSAESTPMQVVTLLNDLYTCFDAVIDNFDVYKVETIGDAYMVVSGLPVRNGRLHACEVARMALALLDAVRSFRIRHRPQEQLRLRIGIHTGPVCAGVVGLKMPRYCLFGDTVNTASRMESNGEALKIHLSSETKAVLEEFGGFELELRGDVEMKGKGKVRTYWLLGERGSSTRG

SEQ ID NO: 2 - protein, 1057 aa, Mus musculus
MPGSRRVRPRLRALLLLPPLLLLRSGHASDLTVAVVLPLTNTSYPWSWARVGPAVELALGRVKARPDLLPGWTVRMVLGSSENAAGVCSDTAAPLAAVDLKWEHSPAVFLGPGCVYSAAPVGRFTAHWRVPLLTAGAPALGIGVKDEYALTTRTGPSHVKLGDFVTALHRRLGWEHQALVLYADRLGDDRPCFFIVEGLYMRVRERLNITVNHQEFVEGDPDHYTKLLRTVQRKGRVIYICSSPDAFRNLMLLALDAGLTGEDYVFFHLDVFGQSLQGAQGPVPRKPWERDDGQDRRARQAFQAAKIITYKEPDNPEYLEFLKQLKLLADKKFNFTMEDGLKNIIPASFHDGLLLYVQAVTETLAQGGTVTDGENITQRMWNRSFQGVTGYLKIDRNGDRDTDFSLWDMDPETGAFRVVLNFNGTSQELMAVSEHRLYWPLGYPPPDIPKCGFDNEDPACNQDHFSTLEVLALVGSLSLVSFLIVSFFIYRKMQLEKELVSELWRVRWEDLQPSSLERHLRSAGSRLTLSGRGSNYGSLLTTEGQFQVFAKTAYYKGNLVAVKRVNRKRIELTRKVLFELKHMRDVQNEHLTRFVGACTDPPNICILTEYCPRGSLQDILENESITLDWMFRYSLTNDIVKGMLFLHNGAIGSHGNLKSSNCVVDGRFVLKITDYGLESFRDPEPEQGHTLFAKKLWTAPELLRMASPPARGSQAGDVYSFGIILQEIALRSGVFYVEGLDLSPKEIIERVTRGEQPPFRPSMDLQSHLEELGQLMQRCWAEDPQERPPFQQIRLALRKFNKENSSNILDNLLSRMEQYANNLEELVEERTQAYLEEKRKAEALLYQILPHSVAEQLKRGETVQAEAFDSVTIYFSDIVGFTALSAESTPMQVVTLLNDLYTCFDAVIDNFDVYKVETIGDAYMVVSGLPVRNGQLHAREVARMALALLDAVRSFRIRHRPQEQLRLRIGIHTGPVCAGVVGLKMPRYCLFGDTVNTASRMESNGEALRIHLSSETKAVLEEFDGFELELRGDVEMKGKGKVRTYWLLGERGCSTRG

SEQ ID NO: 3 - protein, 1057 aa, Rattus norvegicus
MPGSRRVRPRLRALLLLPPLLLLRGGHASDLTVAVVLPLTNTSYPWSWARVGPAVELALARVKARPDLLPGWTVRMVLGSSENAAGVCSDTAAPLAAVDLKWEHSPAVFLGPGCVYSAAPVGRFTAHWRVPLLTAGAPALGIGVKDEYALTTRTGPSHVKLGDFVTALHRRLGWEHQALVLYADRLGDDRPCFFIVEGLYMRVRERLNITVNHQEFVEGDPDHYPKLLRAVRRKGRVIYICSSPDAFRNLMLLALNAGLTGEDYVFFHLDVFGQSLKSAQGLVPQKPWERGDGQDRSARQAFQAAKIITYKEPDNPEYLEFLKQLKLLADKKFNFTVEDGLKNIIPASFHDGLLLYVQAVTETLAQGGTVTDGENITQRMWNRSFQGVTGYLKIDRNGDRDTDFSLWDMDPETGAFRVVLNYNGTSQELMAVSEHKLYWPLGYPPPDVPKCGFDNEDPACNQDHFSTLEVLALVGSLSLISFLIVSFFIYRKMQLEKELVSELWRVRWEDLQPSSLERHLRSAGSRLTLSGRGSNYGSLLTTEGQFQVFAKTAYYKGNLVAVKRVNRKRIELTRKVLFELKHMRDVQNEHLTRFVGACTDPPNICILTEYCPRGSLQDILENESITLDWMFRYSLTNDIVKGMLFLHNGAICSHGNLKSSNCVVDGRFVLKITDYGLESFRDPEPEQGHTLFAKKLWTAPELLRMASPPARGSQAGDVYSFGIILQEIALRSGVFYVEGLDLSPKEIIERVTRGEQPPFRPSMDLQSHLEELGQLMQRCWAEDPQERPPFQQIRLALRKFNKENSSNILDNLLSRMEQYANNLEELVEERTQAYLEEKRKAEALLYQILPHSVAEQLKRGETVQAEAFDSVTIYFSDIVGFTALSAESTPMQVVTLLNDLYTCFDAVIDNFDVYKVETIGDAYMVVSGLPVRNGQLHAREVARMALALLDAVRSFRIRHRPQEQLRLRIGIHTGPVCAGVVGLKMPRYCLFGDTVNTASRMESNGEALKIHLSSETKAVLEEFDGFELELRGDVEMKGKGKVRTYWLLGERGCSTRG

SEQ ID NO: 4 - protein, 10 aa, Artificial Sequence
  source: /note=“Description of Artificial Sequence Synthetic peptide”
GFTFNTHYIH

SEQ ID NO: 5 - protein, 17 aa, Artificial Sequence
  source: /note=“Description of Artificial Sequence Synthetic peptide”
SISGSGSNTYYADSVKG

SEQ ID NO: 6 - protein, 12 aa, Artificial Sequence
  source: /note=“Description of Artificial Sequence Synthetic peptide”
ERGYVYYHMFDP

SEQ ID NO: 7 - protein, 5 aa, Artificial Sequence
  source: /note=“Description of Artificial Sequence Synthetic peptide”
THYIH

SEQ ID NO: 8 - protein, 7 aa, Artificial Sequence
  source: /note=“Description of Artificial Sequence Synthetic peptide”
GFTFNTH

SEQ ID NO: 9 - protein, 6 aa, Artificial Sequence
  source: /note=“Description of Artificial Sequence Synthetic peptide”
SGSGSN

SEQ ID NO: 10 - protein, 8 aa, Artificial Sequence
  source: /note=“Description of Artificial Sequence Synthetic peptide”
GFTFNTHY

SEQ ID NO: 11 - protein, 8 aa, Artificial Sequence
  source: /note=“Description of Artificial Sequence Synthetic peptide”
ISGSGSNT

SEQ ID NO: 12 - protein, 14 aa, Artificial Sequence
  source: /note=“Description of Artificial Sequence Synthetic peptide”
ARERGYVYYHMFDP

SEQ ID NO: 13 - protein, 121 aa, Artificial Sequence
  source: /note=“Description of Artificial Sequence Synthetic polypeptide”
QVQLLESGGGLVQPGGSLRLSCAASGFTFNTHYIHWVRQAPGKGLEWVSSISGSGSNTYYADSVKGRFTISRDNSKNTLYLQMNSLRAEDTAVYYCARERGYVYYHMFDPWGQGTLVTVSS

SEQ ID NO: 14 - DNA, 363 bp, Artificial Sequence
  source: /note=“Description of Artificial Sequence Synthetic polynucleotide”
caggtgcaattgctggaaagcggcggtggcctggtgcagccgggtggcagcctgcgtctgagctgcgcggcgtccggattcacctttaacactcattacatccattgggtgcgccaggcccccggcaaaggtctcgagtgggtttcctctatctctggttctggttctaacacctactatgcggatagcgtgaaaggccgctttaccatcagccgcgataattcgaaaaacaccctgtatctgcaaatgaacagcctgcgtgcggaagatacggccgtgtattattgcgcgcgtgaacgtggttacgtttactaccatatgttcgatccgtggggccaaggcaccctggtgactgttagctca

SEQ ID NO: 15 - protein, 451 aa, Artificial Sequence
  source: /note=“Description of Artificial Sequence Synthetic polypeptide”
QVQLLESGGGLVQPGGSLRLSCAASGFTFNTHYIHWVRQAPGKGLEWVSSISGSGSNTYYADSVKGRFTISRDNSKNTLYLQMNSLRAEDTAVYYCARERGYVYYHMFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1353 bp, Artificial Sequence
  source: /note=“Description of Artificial Sequence Synthetic polynucleotide”
caggtgcaattgctggaaagcggcggtggcctggtgcagccgggtggcagcctgcgtctgagctgcgcggcgtccggattcacctttaacactcattacatccattgggtgcgccaggcccccggcaaaggtctcgagtgggtttcctctatctctggttctggttctaacacctactatgcggatagcgtgaaaggccgctttaccatcagccgcgataattcgaaaaacaccctgtatctgcaaatgaacagcctgcgtgcggaagatacggccgtgtattattgcgcgcgtgaacgtggttacgtttactaccatatgttcgatccgtggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 17 - protein, 12 aa, Artificial Sequence
  source: /note=“Description of Artificial Sequence Synthetic peptide”
RASQSITRNYLA

SEQ ID NO: 18 - protein, 7 aa, Artificial Sequence
  source: /note=“Description of Artificial Sequence Synthetic peptide”
GASSRAT

SEQ ID NO: 19 - protein, 9 aa, Artificial Sequence
  source: /note=“Description of Artificial Sequence Synthetic peptide”
QQHSMYPRT

SEQ ID NO: 20 - protein, 8 aa, Artificial Sequence
  source: /note=“Description of Artificial Sequence Synthetic peptide”
SQSITRNY

SEQ ID NO: 21 - protein, 3 aa, Artificial Sequence
  source: /note=“Description of Artificial Sequence Synthetic peptide”
GAS

SEQ ID NO: 22 - protein, 6 aa, Artificial Sequence
  source: /note=“Description of Artificial Sequence Synthetic peptide”
HSMYPR

SEQ ID NO: 23 - protein, 7 aa, Artificial Sequence
  source: /note=“Description of Artificial Sequence Synthetic peptide”
QSITRNY

SEQ ID NO: 24 - protein, 108 aa, Artificial Sequence
  source: /note=“Description of Artificial Sequence Synthetic polypeptide”
DIVLTQSPATLSLSPGERATLSCRASQSITRNYLAWYQQKPGQAPRLLIYGASSRATGIPARFSGSGSGTDFTLTISSLEPEDFAVYYCQQHSMYPRTFGQGTKVEIK

SEQ ID NO: 25 - DNA, 324 bp, Artificial Sequence
  source: /note=“Description of Artificial Sequence Synthetic polynucleotide”
gatatcgtgctgacccagagcccggcgaccctgagcctgagcccgggtgaacgtgccaccctgagctgcagagcgagccagtctatcactcgtaactacctggcttggtaccagcagaaaccgggccaggccccgcgtctattaatctacggtgcttcttctcgtgcgaccggcattccggcgcgttttagcggcagcggatccggcaccgatttcaccctgaccattagcagcctggaaccggaagactttgcggtgtattattgccagcagcattctatgtacccgcgtacctttggccagggcacgaaagttgaaattaaa

SEQ ID NO: 26 - protein, 215 aa, Artificial Sequence
  source: /note=“Description of Artificial Sequence Synthetic polypeptide”
DIVLTQSPATLSLSPGERATLSCRASQSITRNYLAWYQQKPGQAPRLLIYGASSRATGIPARFSGSGSGTDFTLTISSLEPEDFAVYYCQQHSMYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 645 bp, Artificial Sequence
  source: /note=“Description of Artificial Sequence Synthetic polynucleotide”
gatatcgtgctgacccagagcccggcgaccctgagcctgagcccgggtgaacgtgccaccctgagctgcagagcgagccagtctatcactcgtaactacctggcttggtaccagcagaaaccgggccaggccccgcgtctattaatctacggtgcttcttctcgtgcgaccggcattccggcgcgttttagcggcagcggatccggcaccgatttcaccctgaccattagcagcctggaaccggaagactttgcggtgtattattgccagcagcattctatgtacccgcgtacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 28 - protein, 10 aa, Artificial Sequence
  source: /note=“Description of Artificial Sequence Synthetic peptide”
GFTFSSYWMN

SEQ ID NO: 29 - protein, 17 aa, Artificial Sequence
  source: /note=“Description of Artificial Sequence Synthetic peptide”
AISSDGSYTYYADSVKG

SEQ ID NO: 30 - protein, 16 aa, Artificial Sequence
  source: /note=“Description of Artificial Sequence Synthetic peptide”
DRYSMIYSYGAGAFDY

SEQ ID NO: 31 - protein, 5 aa, Artificial Sequence
  source: /note=“Description of Artificial Sequence Synthetic peptide”
SYWMN

SEQ ID NO: 32 - protein, 7 aa, Artificial Sequence
  source: /note=“Description of Artificial Sequence Synthetic peptide”
GFTFSSY

SEQ ID NO: 33 - protein, 6 aa, Artificial Sequence
  source: /note=“Description of Artificial Sequence Synthetic peptide”
SSDGSY

SEQ ID NO: 34 - protein, 8 aa, Artificial Sequence
  source: /note=“Description of Artificial Sequence Synthetic peptide”
GFTFSSYW

SEQ ID NO: 35 - protein, 8 aa, Artificial Sequence
  source: /note=“Description of Artificial Sequence Synthetic peptide”
ISSDGSYT

SEQ ID NO: 36 - protein, 18 aa, Artificial Sequence
  source: /note=“Description of Artificial Sequence Synthetic peptide”
ARDRYSMIYSYGAGAFDY

SEQ ID NO: 37 - protein, 125 aa, Artificial Sequence
  source: /note=“Description of Artificial Sequence Synthetic polypeptide”
QVQLLESGGGLVQPGGSLRLSCAASGFTFSSYWMNWVRQAPGKGLEWVSAISSDGSYTYYADSVKGRFTISRDNSKNTLYLQMNSLRAEDTAVYYCARDRYSMIYSYGAGAFDYWGQGTLVTVSS

SEQ ID NO: 38 - DNA, 375 bp, Artificial Sequence
  source: /note=“Description of Artificial Sequence Synthetic polynucleotide”
caggtgcaattgctggaaagcggcggtggcctggtgcagccgggtggcagcctgcgtctgagctgcgcggcgtccggattcaccttttcttcttactggatgaactgggtgcgccaggccccgggcaaaggtctcgagtgggtttccgctatctcttctgacggttcttacacctactatgcggatagcgtgaaaggccgctttaccatcagccgcgataattcgaaaaacaccctgtatctgcaaatgaacagcctgcgtgcggaagatacggccgtgtattattgcgcgcgtgaccgttactctatgatctactcttacggtgctggtgctttcgattactggggccaaggcaccctggtgactgttagctca

SEQ ID NO: 39 - protein, 455 aa, Artificial Sequence
  source: /note=“Description of Artificial Sequence Synthetic polypeptide”
QVQLLESGGGLVQPGGSLRLSCAASGFTFSSYWMNWVRQAPGKGLEWVSAISSDGSYTYYADSVKGRFTISRDNSKNTLYLQMNSLRAEDTAVYYCARDRYSMIYSYGAGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 1365 bp, Artificial Sequence
  source: /note=“Description of Artificial Sequence Synthetic polynucleotide”
caggtgcaattgctggaaagcggcggtggcctggtgcagccgggtggcagcctgcgtctgagctgcgcggcgtccggattcaccttttcttcttactggatgaactgggtgcgccaggccccgggcaaaggtctcgagtgggtttccgctatctcttctgacggttcttacacctactatgcggatagcgtgaaaggccgctttaccatcagccgcgataattcgaaaaacaccctgtatctgcaaatgaacagcctgcgtgcggaagatacggccgtgtattattgcgcgcgtgaccgttactctatgatctactcttacggtgctggtgctttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 41 - protein, 11 aa, Artificial Sequence
  source: /note=“Description of Artificial Sequence Synthetic peptide”
RASQGISSYLA

SEQ ID NO: 42 - protein, 7 aa, Artificial Sequence
  source: /note=“Description of Artificial Sequence Synthetic peptide”
TASTLQS

SEQ ID NO: 43 - protein, 9 aa, Artificial Sequence
  source: /note=“Description of Artificial Sequence Synthetic peptide”
MQSYEKPRT

SEQ ID NO: 44 - protein, 7 aa, Artificial Sequence
  source: /note=“Description of Artificial Sequence Synthetic peptide”
SQGISSY

SEQ ID NO: 45 - protein, 3 aa, Artificial Sequence
  source: /note=“Description of Artificial Sequence Synthetic peptide”
TAS

SEQ ID NO: 46 - protein, 6 aa, Artificial Sequence
  source: /note=“Description of Artificial Sequence Synthetic peptide”
SYEKPR

SEQ ID NO: 47 - protein, 6 aa, Artificial Sequence
  source: /note=“Description of Artificial Sequence Synthetic peptide”
QGISSY

SEQ ID NO: 48 - protein, 107 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCMQSYEKPRTFGQGTKVEIK

SEQ ID NO: 49 - DNA, 321 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgcatgcagtcttacgaaaaaccgcgtacctttggccagggcacgaaagttgaaattaaa

SEQ ID NO: 50 - protein, 214 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCMQSYEKPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - DNA, 642 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgcatgcagtcttacgaaaaaccgcgtacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 52 - protein, 10 aa, Artificial Sequence
  source: /note=“Description of Artificial Sequence Synthetic peptide”
GYSFSNYWIG

SEQ ID NO: 53 - protein, 17 aa, Artificial Sequence
  source: /note=“Description of Artificial Sequence Synthetic peptide”
IIYPDVSYTRYSPSFQG

SEQ ID NO: 54 - protein, 10 aa, Artificial Sequence
  source: /note=“Description of Artificial Sequence Synthetic peptide”
YWSEAYTFDY

SEQ ID NO: 55 - protein, 5 aa, Artificial Sequence
  source: /note=“Description of Artificial Sequence Synthetic peptide”
NYWIG

SEQ ID NO: 56 - protein, 7 aa, Artificial Sequence
  source: /note=“Description of Artificial Sequence Synthetic peptide”
GYSFSNY

SEQ ID NO: 57 - protein, 6 aa, Artificial Sequence
  source: /note=“Description of Artificial Sequence Synthetic peptide”
YPDVSY

SEQ ID NO: 58 - protein, 8 aa, Artificial Sequence
  source: /note=“Description of Artificial Sequence Synthetic peptide”
GYSFSNYW

SEQ ID NO: 59 - protein, 8 aa, Artificial Sequence
  source: /note=“Description of Artificial Sequence Synthetic peptide”
IYPDVSYT

SEQ ID NO: 60 - protein, 12 aa, Artificial Sequence
  source: /note=“Description of Artificial Sequence Synthetic peptide”
ARYWSEAYTFDY

SEQ ID NO: 61 - protein, 119 aa, Artificial Sequence
  source: /note=“Description of Artificial Sequence Synthetic polypeptide”
QVQLVQSGAEVKKPGESLKISCKGSGYSFSNYWIGWVRQMPGKGLEWMGIIYPDVSYTRYSPSFQGQVTISADKSISTAYLQWSSLKASDTAMYYCARYWSEAYTFDYWGQGTLVTVSS

SEQ ID NO: 62 - DNA, 357 bp, Artificial Sequence
  source: /note=“Description of Artificial Sequence Synthetic polynucleotide”
caggtgcaattggtgcagagcggtgcggaagtgaaaaaaccgggcgaaagcctgaaaattagctgcaaaggctccggatatagcttctctaactactggatcggttgggtgcgccagatgccgggcaaaggtctcgagtggatgggcatcatctacccggacgttagctacacccgttatagcccgagctttcagggccaggtgaccattagcgcggataaaagcatcagcaccgcgtatctgcaatggagcagcctgaaagcgagcgataccgcgatgtattattgcgcgcgttactggtctgaagcttacactttcgattactggggccaaggcaccctggtgactgttagctca

SEQ ID NO: 63 - protein, 449 aa, Artificial Sequence
  source: /note=“Description of Artificial Sequence Synthetic polypeptide”
QVQLVQSGAEVKKPGESLKISCKGSGYSFSNYWIGWVRQMPGKGLEWMGIIYPDVSYTRYSPSFQGQVTISADKSISTAYLQWSSLKASDTAMYYCARYWSEAYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - DNA, 1347 bp, Artificial Sequence
  source: /note=“Description of Artificial Sequence Synthetic polynucleotide”
caggtgcaattggtgcagagcggtgcggaagtgaaaaaaccgggcgaaagcctgaaaattagctgcaaaggctccggatatagcttctctaactactggatcggttgggtgcgccagatgccgggcaaaggtctcgagtggatgggcatcatctacccggacgttagctacacccgttatagcccgagctttcagggccaggtgaccattagcgcggataaaagcatcagcaccgcgtatctgcaatggagcagcctgaaagcgagcgataccgcgatgtattattgcgcgcgttactggtctgaagcttacactttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 65 - protein, 11 aa, Artificial Sequence
  source: /note=“Description of Artificial Sequence Synthetic peptide”
SGDNIRKKYVF

SEQ ID NO: 66 - protein, 7 aa, Artificial Sequence
  source: /note=“Description of Artificial Sequence Synthetic peptide”
GDNDRPS

SEQ ID NO: 67 - protein, 11 aa, Artificial Sequence
  source: /note=“Description of Artificial Sequence Synthetic peptide”
GTYTLLFTSKV

SEQ ID NO: 68 - protein, 7 aa, Artificial Sequence
  source: /note=“Description of Artificial Sequence Synthetic peptide”
DNIRKKY

SEQ ID NO: 69 - protein, 3 aa, Artificial Sequence
  source: /note=“Description of Artificial Sequence Synthetic peptide”
GDN

SEQ ID NO: 70 - protein, 8 aa, Artificial Sequence
  source: /note=“Description of Artificial Sequence Synthetic peptide”
YTLLFTSK

SEQ ID NO: 71 - protein, 6 aa, Artificial Sequence
  source: /note=“Description of Artificial Sequence Synthetic peptide”
NIRKKY

SEQ ID NO: 72 - protein, 108 aa, Artificial Sequence
  source: /note=“Description of Artificial Sequence Synthetic polypeptide”
DIELTQPPSVSVSPGQTASITCSGDNIRKKYVFWYQQKPGQAPVLVIYGDNDRPSGIPERFSGSNSGNTATLTISGTQAEDEADYYCGTYTLLFTSKVFGGGTKLTVL

SEQ ID NO: 73 - DNA, 324 bp, Artificial Sequence
  source: /note=“Description of Artificial Sequence Synthetic polynucleotide”
gatatcgaactgacccagccgccgagcgtgagcgtgagtccgggccagaccgcgagcattacctgtagcggcgataacatccgtaaaaaatacgttttctggtaccagcagaaaccgggccaggcgccggtgctggtgatctacggtgacaacgaccgtccgagcggcatcccggaacgttttagcggatccaacagcggcaacaccgcgaccctgaccattagcggcacccaggcggaagacgaagcggattattactgcggtacttacactctgctgttcacttctaaagtgtttggcggcggcacgaagttaaccgtccta

SEQ ID NO: 74 - protein, 214 aa, Artificial Sequence
  source: /note=“Description of Artificial Sequence Synthetic polypeptide”
DIELTQPPSVSVSPGQTASITCSGDNIRKKYVFWYQQKPGQAPVLVIYGDNDRPSGIPERFSGSNSGNTATLTISGTQAEDEADYYCGTYTLLFTSKVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - DNA, 642 bp, Artificial Sequence
  source: /note=“Description of Artificial Sequence Synthetic polynucleotide”
gatatcgaactgacccagccgccgagcgtgagcgtgagtccgggccagaccgcgagcattacctgtagcggcgataacatccgtaaaaaatacgttttctggtaccagcagaaaccgggccaggcgccggtgctggtgatctacggtgacaacgaccgtccgagcggcatcccggaacgttttagcggatccaacagcggcaacaccgcgaccctgaccattagcggcacccaggcggaagacgaagcggattattactgcggtacttacactctgctgttcacttctaaagtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 76 - protein, 10 aa, Artificial Sequence
  source: /note=“Description of Artificial Sequence Synthetic peptide”
GYSFTSYWIA

SEQ ID NO: 77 - protein, 17 aa, Artificial Sequence
  source: /note=“Description of Artificial Sequence Synthetic peptide”
RIDPDNSYTRYSPSFQG

SEQ ID NO: 78 - protein, 17 aa, Artificial Sequence
  source: /note=“Description of Artificial Sequence Synthetic peptide”
WLSPGYALGEQPAGMDH

SEQ ID NO: 79 - protein, 5 aa, Artificial Sequence
  source: /note=“Description of Artificial Sequence Synthetic peptide”
SYWIA

SEQ ID NO: 80 - protein, 7 aa, Artificial Sequence
  source: /note=“Description of Artificial Sequence Synthetic peptide”
GYSFTSY

SEQ ID NO: 81 - protein, 6 aa, Artificial Sequence
  source: /note=“Description of Artificial Sequence Synthetic peptide”
DPDNSY

SEQ ID NO: 82 - protein, 8 aa, Artificial Sequence
  source: /note=“Description of Artificial Sequence Synthetic peptide”
GYSFTSYW

SEQ ID NO: 83 - protein, 8 aa, Artificial Sequence
  source: /note=“Description of Artificial Sequence Synthetic peptide”
IDPDNSYT

SEQ ID NO: 84 - protein, 19 aa, Artificial Sequence
  source: /note=“Description of Artificial Sequence Synthetic peptide”
ARWLSPGYALGEQPAGMDH

SEQ ID NO: 85 - protein, 126 aa, Artificial Sequence
  source: /note=“Description of Artificial Sequence Synthetic polypeptide”
QVQLVQSGAEVKKPGESLKISCKGSGYSFTSYWIAWVRQMPGKGLEWMGRIDPDNSYTRYSPSFQGQVTISADKSISTAYLQWSSLKASDTAMYYCARWLSPGYALGEQPAGMDHWGQGTLVTVSS

SEQ ID NO: 86 - DNA, 378 bp, Artificial Sequence
  source: /note=“Description of Artificial Sequence Synthetic polynucleotide”
caggtgcaattggtgcagagcggtgcggaagtgaaaaaaccgggcgaaagcctgaaaattagctgcaaaggctccggatatagcttcacttcttactggatcgcttgggtgcgccagatgccgggcaaaggtctcgagtggatgggccgtatcgacccggacaacagctacacccgttatagcccgagctttcagggccaggtgaccattagcgcggataaaagcatcagcaccgcgtatctgcaatggagcagcctgaaagcgagcgataccgcgatgtattattgcgcgcgttggctgtctccgggttacgctctgggtgaacagccggctggtatggatcattggggccaaggcaccctggtgactgttagctca

SEQ ID NO: 87 - protein, 456 aa, Artificial Sequence
  source: /note=“Description of Artificial Sequence Synthetic polypeptide”
QVQLVQSGAEVKKPGESLKISCKGSGYSFTSYWIAWVRQMPGKGLEWMGRIDPDNSYTRYSPSFQGQVTISADKSISTAYLQWSSLKASDTAMYYCARWLSPGYALGEQPAGMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 1368 bp, Artificial Sequence
  source: /note=“Description of Artificial Sequence Synthetic polynucleotide”
caggtgcaattggtgcagagcggtgcggaagtgaaaaaaccgggcgaaagcctgaaaattagctgcaaaggctccggatatagcttcacttcttactggatcgcttgggtgcgccagatgccgggcaaaggtctcgagtggatgggccgtatcgacccggacaacagctacacccgttatagcccgagctttcagggccaggtgaccattagcgcggataaaagcatcagcaccgcgtatctgcaatggagcagcctgaaagcgagcgataccgcgatgtattattgcgcgcgttggctgtctccgggttacgctctgggtgaacagccggctggtatggatcattggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 89 - protein, 14 aa, Artificial Sequence
  source: /note=“Description of Artificial Sequence Synthetic peptide”
TGSSSNIGAGYAVH

SEQ ID NO: 90 - protein, 7 aa, Artificial Sequence
  source: /note=“Description of Artificial Sequence Synthetic peptide”
SNNKRPS

SEQ ID NO: 91 - protein, 11 aa, Artificial Sequence
  source: /note=“Description of Artificial Sequence Synthetic peptide”
QSYDLQKSSRV

SEQ ID NO: 92 - protein, 10 aa, Artificial Sequence
  source: /note=“Description of Artificial Sequence Synthetic peptide”
SSSNIGAGYA

SEQ ID NO: 93 - protein, 3 aa, Artificial Sequence
  source: /note=“Description of Artificial Sequence Synthetic peptide”
SNN

SEQ ID NO: 94 - protein, 8 aa, Artificial Sequence
  source: /note=“Description of Artificial Sequence Synthetic peptide”
YDLQKSSR

SEQ ID NO: 95 - protein, 9 aa, Artificial Sequence
  source: /note=“Description of Artificial Sequence Synthetic peptide”
SSNIGAGYA

SEQ ID NO: 96 - protein, 111 aa, Artificial Sequence
  source: /note=“Description of Artificial Sequence Synthetic polypeptide”
DIVLTQPPSVSGAPGQRVTISCTGSSSNIGAGYAVHWYQQLPGTAPKLLIYSNNKRPSGVPDRFSGSKSGTSASLAITGLQAEDEADYYCQSYDLQKSSRVFGGGTKLTVL

SEQ ID NO: 97 - DNA, 333 bp, Artificial Sequence
  source: /note=“Description of Artificial Sequence Synthetic polynucleotide”
gatatcgtgctgacccagccgccgagcgtgagcggtgcaccgggccagcgcgtgaccattagctgtaccggcagcagcagcaacattggtgctggttacgctgtgcattggtaccagcagctgccgggcacggcgccgaaactgctgatctactctaacaacaaacgcccgagcggcgtgccggatcgctttagcggatccaaaagcggcaccagcgccagcctggcgattaccggcctgcaagcagaagacgaagcggattattactgccagtcttacgacctgcagaaatcttctcgtgtgtttggcggcggcacgaagttaaccgtccta

SEQ ID NO: 98 - protein, 217 aa, Artificial Sequence
  source: /note=“Description of Artificial Sequence Synthetic polypeptide”
DIVLTQPPSVSGAPGQRVTISCTGSSSNIGAGYAVHWYQQLPGTAPKLLIYSNNKRPSGVPDRFSGSKSGTSASLAITGLQAEDEADYYCQSYDLQKSSRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 99 - DNA, 651 bp, Artificial Sequence
  source: /note=“Description of Artificial Sequence Synthetic polynucleotide”
gatatcgtgctgacccagccgccgagcgtgagcggtgcaccgggccagcgcgtgaccattagctgtaccggcagcagcagcaacattggtgctggttacgctgtgcattggtaccagcagctgccgggcacggcgccgaaactgctgatctactctaacaacaaacgcccgagcggcgtgccggatcgctttagcggatccaaaagcggcaccagcgccagcctggcgattaccggcctgcaagcagaagacgaagcggattattactgccagtcttacgacctgcagaaatcttctcgtgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 100 - protein, 17 aa, Artificial Sequence
  source: /note=“Description of Artificial Sequence Synthetic peptide”
SISSSGQSTYYADSVKG

SEQ ID NO: 101 - protein, 6 aa, Artificial Sequence
  source: /note=“Description of Artificial Sequence Synthetic peptide”
SSSGQS

SEQ ID NO: 102 - protein, 8 aa, Artificial Sequence
  source: /note=“Description of Artificial Sequence Synthetic peptide”
ISSSGQST

SEQ ID NO: 103 - protein, 121 aa, Artificial Sequence
  source: /note=“Description of Artificial Sequence Synthetic polypeptide”
EVQLLESGGGLVQPGGSLRLSCAASGFTFNTHYIHWVRQAPGKGLEWVSSISSSGQSTYYADSVKGRFTISRDNSKNTLYLQMNSLRAEDTAVYYCARERGYVYYHMFDPWGQGTLVTVSS

SEQ ID NO: 104 - DNA, 363 bp, Artificial Sequence
  source: /note=“Description of Artificial Sequence Synthetic polynucleotide”
gaggtgcaattgctggaaagcggcggtggcctggtgcagccgggtggcagcctgcgtctgagctgcgcggcgtccggattcacctttaacactcattacatccattgggtgcgccaggcccccggcaaaggtctcgagtgggtttcctctatctcttcttctggccagtctacttactatgcggatagcgtgaaaggccgctttaccatcagccgcgataattcgaaaaacaccctgtatctgcaaatgaacagcctgcgtgcggaagatacggccgtgtattattgcgcgcgtgaacgtggttacgtttactaccatatgttcgatccgtggggccaaggcaccctggtgactgttagctca

SEQ ID NO: 105 - protein, 451 aa, Artificial Sequence
  source: /note=“Description of Artificial Sequence Synthetic polypeptide”
EVQLLESGGGLVQPGGSLRLSCAASGFTFNTHYIHWVRQAPGKGLEWVSSISSSGQSTYYADSVKGRFTISRDNSKNTLYLQMNSLRAEDTAVYYCARERGYVYYHMFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - DNA, 1353 bp, Artificial Sequence
  source: /note=“Description of Artificial Sequence Synthetic polynucleotide”
gaggtgcaattgctggaaagcggcggtggcctggtgcagccgggtggcagcctgcgtctgagctgcgcggcgtccggattcacctttaacactcattacatccattgggtgcgccaggcccccggcaaaggtctcgagtgggtttcctctatctcttcttctggccagtctacttactatgcggatagcgtgaaaggccgctttaccatcagccgcgataattcgaaaaacaccctgtatctgcaaatgaacagcctgcgtgcggaagatacggccgtgtattattgcgcgcgtgaacgtggttacgtttactaccatatgttcgatccgtggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 107 - DNA, 363 bp, Artificial Sequence
  source: /note=“Description of Artificial Sequence Synthetic polynucleotide”
gaagtgcagctgcttgagtccgggggtggactggtgcagcccggaggatccctgcgcctgagctgcgctgcatccggcttcaccttcaacacgcactacatccattgggtcagacaggccccaggaaaaggcctggaatgggtgtcctccatctcctcgtcggggcagtcaacctactacgcggactccgtcaagggccggtttaccattagccgggacaacagcaagaataccctgtacctccaaatgaactcgctgagggccgaagataccgccgtgtattactgtgcccgcgagagaggctacgtgtactaccacatgttcgacccgtggggacagggtactctcgtgactgtgtcttct

SEQ ID NO: 108 - protein, 451 aa, Artificial Sequence
  source: /note=“Description of Artificial Sequence Synthetic polypeptide”
EVQLLESGGGLVQPGGSLRLSCAASGFTFNTHYIHWVRQAPGKGLEWVSSISSSGQSTYYADSVKGRFTISRDNSKNTLYLQMNSLRAEDTAVYYCARERGYVYYHMFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - DNA, 1353 bp, Artificial Sequence
  source: /note=“Description of Artificial Sequence Synthetic polynucleotide”
gaagtgcagctgcttgagtccgggggtggactggtgcagcccggaggatccctgcgcctgagctgcgctgcatccggcttcaccttcaacacgcactacatccattgggtcagacaggccccaggaaaaggcctggaatgggtgtcctccatctcctcgtcggggcagtcaacctactacgcggactccgtcaagggccggtttaccattagccgggacaacagcaagaataccctgtacctccaaatgaactcgctgagggccgaagataccgccgtgtattactgtgcccgcgagagaggctacgtgtactaccacatgttcgacccgtggggacagggtactctcgtgactgtgtcttctgcgagcactaagggcccgtcagtgttcccgctggctccatcgtcgaagtccacctccggaggaaccgcagcactcggttgcctggtcaaggactacttccctgagccagtgaccgtgtcgtggaacagcggagccctgacttccggcgtgcacacttttcccgcggtgctgcagtcctccggtctgtactccctttcgtccgtggtcaccgtgccgtcgtctagcctgggcacccagacctacatctgcaacgtgaaccacaagccgtccaacaccaaagtggataagcgggtggagccgaagtcctgcgataagacacacacgtgcccgccatgtccagcgcctgaattgcttggcggaccttccgtgttcctgttcccgcctaagcccaaggacaccttgatgattagccggactcccgaagtcacctgtgtggtggtggcagtgtcccacgaggaccccgaggtcaagtttaattggtacgtggacggcgtcgaagtgcacaacgccaagactaagccccgggaggaacagtacaacagcacctaccgggtcgtgtccgtgctgaccgtgctgcaccaggactggctgaatgggaaagagtacaagtgcaaagtgtccaacaaggccttggccgctcctatcgaaaaaactatcagcaaggctaagggacagccgagggaaccccaagtctacaccctgcccccttcacgcgaagagatgaccaagaatcaagtgtcgctgacctgcctcgtcaagggattctacccctccgacattgcggtggagtgggagtccaacggccagcccgagaacaactacaagactactccgcccgtgctggactccgacggcagcttcttcctgtattccaagctgaccgtggacaagtcccggtggcagcaaggaaacgtgttctcctgctcggtcatgcacgaagccctgcacaaccactatacgcagaagtccctgtccttgagcccggggaaa

SEQ ID NO: 110 - DNA, 324 bp, Artificial Sequence
  source: /note=“Description of Artificial Sequence Synthetic polynucleotide”
gacatcgtgctgactcagtcccctgcgactctgagcctgtcaccgggagaacgggccaccctctcttgccgcgcctcccaatccattactcggaactacctggcctggtatcagcagaagccaggacaggcccctaggcttctgatctacggggccagctcaagagcaactggcatcccggctcgcttctccggttcgggaagcggcaccgacttcaccctgacaatttcgtccctcgaacccgaggatttcgccgtgtactactgccaacagcactccatgtacccccggacctttgggcagggaaccaaagtcgagatcaag

SEQ ID NO: 111 - DNA, 645 bp, Artificial Sequence
  source: /note=“Description of Artificial Sequence Synthetic polynucleotide”
gacatcgtgctgactcagtcccctgcgactctgagcctgtcaccgggagaacgggccaccctctcttgccgcgcctcccaatccattactcggaactacctggcctggtatcagcagaagccaggacaggcccctaggcttctgatctacggggccagctcaagagcaactggcatcccggctcgcttctccggttcgggaagcggcaccgacttcaccctgacaatttcgtccctcgaacccgaggatttcgccgtgtactactgccaacagcactccatgtacccccggacctttgggcagggaaccaaagtcgagatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 112 - protein, 10 aa, Artificial Sequence
  source: /note=“Description of Artificial Sequence Synthetic peptide”
GFTFSTHYIH

SEQ ID NO: 113 - protein, 7 aa, Artificial Sequence
  source: /note=“Description of Artificial Sequence Synthetic peptide”
GFTFSTH

SEQ ID NO: 114 - protein, 8 aa, Artificial Sequence
  source: /note=“Description of Artificial Sequence Synthetic peptide”
GFTFSTHY

SEQ ID NO: 115 - protein, 121 aa, Artificial Sequence
  source: /note=“Description of Artificial Sequence Synthetic polypeptide”
EVQLLESGGGLVQPGGSLRLSCAASGFTFSTHYIHWVRQAPGKGLEWVSSISSSGQSTYYADSVKGRFTISRDNSKNTLYLQMNSLRAEDTAVYYCARERGYVYYHMFDPWGQGTLVTVSS

SEQ ID NO: 116 - DNA, 363 bp, Artificial Sequence
  source: /note=“Description of Artificial Sequence Synthetic polynucleotide”
gaagtgcagctgcttgagtccgggggtggactggtgcagcccggaggatccctgcgcctgagctgcgctgcatccggcttcaccttcagcacgcactacatccattgggtcagacaggccccaggaaaaggcctggaatgggtgtcctccatctcctcgtcggggcagtcaacctactacgcggactccgtcaagggccggtttaccattagccgggacaacagcaagaataccctgtacctccaaatgaactcgctgagggccgaagataccgccgtgtattactgtgcccgcgagagaggctacgtgtactaccacatgttcgacccgtggggacagggtactctcgtgactgtgtcttct

SEQ ID NO: 117 - protein, 451 aa, Artificial Sequence
  source: /note=“Description of Artificial Sequence Synthetic polypeptide”
EVQLLESGGGLVQPGGSLRLSCAASGFTFSTHYIHWVRQAPGKGLEWVSSISSSGQSTYYADSVKGRFTISRDNSKNTLYLQMNSLRAEDTAVYYCARERGYVYYHMFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - DNA, 1353 bp, Artificial Sequence
  source: /note=“Description of Artificial Sequence Synthetic polynucleotide”
gaagtgcagctgcttgagtccgggggtggactggtgcagcccggaggatccctgcgcctgagctgcgctgcatccggcttcaccttcagcacgcactacatccattgggtcagacaggccccaggaaaaggcctggaatgggtgtcctccatctcctcgtcggggcagtcaacctactacgcggactccgtcaagggccggtttaccattagccgggacaacagcaagaataccctgtacctccaaatgaactcgctgagggccgaagataccgccgtgtattactgtgcccgcgagagaggctacgtgtactaccacatgttcgacccgtggggacagggtactctcgtgactgtgtcttctgcgagcactaagggcccgtcagtgttcccgctggctccatcgtcgaagtccacctccggaggaaccgcagcactcggttgcctggtcaaggactacttccctgagccagtgaccgtgtcgtggaacagcggagccctgacttccggcgtgcacacttttcccgcggtgctgcagtcctccggtctgtactccctttcgtccgtggtcaccgtgccgtcgtctagcctgggcacccagacctacatctgcaacgtgaaccacaagccgtccaacaccaaagtggataagcgggtggagccgaagtcctgcgataagacacacacgtgcccgccatgtccagcgcctgaattgcttggcggaccttccgtgttcctgttcccgcctaagcccaaggacaccttgatgattagccggactcccgaagtcacctgtgtggtggtggcagtgtcccacgaggaccccgaggtcaagtttaattggtacgtggacggcgtcgaagtgcacaacgccaagactaagccccgggaggaacagtacaacagcacctaccgggtcgtgtccgtgctgaccgtgctgcaccaggactggctgaatgggaaagagtacaagtgcaaagtgtccaacaaggccttggccgctcctatcgaaaaaactatcagcaaggctaagggacagccgagggaaccccaagtctacaccctgcccccttcacgcgaagagatgaccaagaatcaagtgtcgctgacctgcctcgtcaagggattctacccctccgacattgcggtggagtgggagtccaacggccagcccgagaacaactacaagactactccgcccgtgctggactccgacggcagcttcttcctgtattccaagctgaccgtggacaagtcccggtggcagcaaggaaacgtgttctcctgctcggtcatgcacgaagccctgcacaaccactatacgcagaagtccctgtccttgagcccggggaaa

SEQ ID NO: 119 - protein, 17 aa, Artificial Sequence
  source: /note=“Description of Artificial Sequence Synthetic peptide”
VIESKGNYIFYADSVKG

SEQ ID NO: 120 - protein, 6 aa, Artificial Sequence
  source: /note=“Description of Artificial Sequence Synthetic peptide”
ESKGNY

SEQ ID NO: 121 - protein, 8 aa, Artificial Sequence
  source: /note=“Description of Artificial Sequence Synthetic peptide”
IESKGNYI

SEQ ID NO: 122 - protein, 125 aa, Artificial Sequence
  source: /note=“Description of Artificial Sequence Synthetic polypeptide”
EVQLLESGGGLVQPGGSLRLSCAASGFTFSSYWMNWVRQAPGKGLEWVSVIESKGNYIFYADSVKGRFTISRDNSKNTLYLQMNSLRAEDTAVYYCARDRYSMIYSYGAGAFDYWGQGTLVTVSS

SEQ ID NO: 123 - DNA, 375 bp, Artificial Sequence
  source: /note=“Description of Artificial Sequence Synthetic polynucleotide”
gaggtgcaattgctggaaagcggcggtggcctggtgcagccgggtggcagcctgcgtctgagctgcgcggcgtccggattcaccttttcttcttactggatgaactgggtgcgccaggccccgggcaaaggtctcgagtgggtttccgttatcgaatctaaaggcaactacatcttctatgcggatagcgtgaaaggccgctttaccatcagccgcgataattcgaaaaacaccctgtatctgcaaatgaacagcctgcgtgcggaagatacggccgtgtattattgcgcgcgtgaccgttactctatgatctactcttacggtgctggtgctttcgattactggggccaaggcaccctggtgactgttagctca

SEQ ID NO: 124 - protein, 455 aa, Artificial Sequence
  source: /note=“Description of Artificial Sequence Synthetic polypeptide”
EVQLLESGGGLVQPGGSLRLSCAASGFTFSSYWMNWVRQAPGKGLEWVSVIESKGNYIFYADSVKGRFTISRDNSKNTLYLQMNSLRAEDTAVYYCARDRYSMIYSYGAGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - DNA, 1365 bp, Artificial Sequence
  source: /note=“Description of Artificial Sequence Synthetic polynucleotide”
gaggtgcaattgctggaaagcggcggtggcctggtgcagccgggtggcagcctgcgtctgagctgcgcggcgtccggattcaccttttcttcttactggatgaactgggtgcgccaggccccgggcaaaggtctcgagtgggtttccgttatcgaatctaaaggcaactacatcttctatgcggatagcgtgaaaggccgctttaccatcagccgcgataattcgaaaaacaccctgtatctgcaaatgaacagcctgcgtgcggaagatacggccgtgtattattgcgcgcgtgaccgttactctatgatctactcttacggtgctggtgctttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 126 - protein, 9 aa, Artificial Sequence
  source: /note=“Description of Artificial Sequence Synthetic peptide”
QQEWVKPRT

SEQ ID NO: 127 - protein, 6 aa, Artificial Sequence
  source: /note=“Description of Artificial Sequence Synthetic peptide”
EWVKPR

SEQ ID NO: 128 - protein, 107 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQEWVKPRTFGQGTKVEIK

SEQ ID NO: 129 - DNA, 321 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcaggaatgggttaaaccgcgtacctttggccagggcacgaaagttgaaattaaa

SEQ ID NO: 130 - protein, 214 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQEWVKPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - DNA, 642 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcaggaatgggttaaaccgcgtacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 132 - DNA, 375 bp, Artificial Sequence
  source: /note=“Description of Artificial Sequence Synthetic polynucleotide”
caggtgcaattgctggaaagcggcggtggcctggtgcagccgggtggcagcctgcgtctgagctgcgcggcgtccggattcaccttttcttcttactggatgaactgggtgcgccaggccccaggcaaaggtctcgagtgggtttccgctatctcttctgacggttcttacacctactatgcggatagcgtgaaaggccgctttaccatcagccgcgataattcgaaaaacaccctgtatctgcaaatgaacagcctgcgtgcggaagatacggccgtgtattattgcgcgcgtgaccgttactctatgatctactcttacggtgctggtgctttcgattactggggccaaggcaccctggtgactgttagctca

SEQ ID NO: 133 - DNA, 1365 bp, Artificial Sequence
  source: /note=“Description of Artificial Sequence Synthetic polynucleotide”
caggtgcaattgctggaaagcggcggtggcctggtgcagccgggtggcagcctgcgtctgagctgcgcggcgtccggattcaccttttcttcttactggatgaactgggtgcgccaggccccaggcaaaggtctcgagtgggtttccgctatctcttctgacggttcttacacctactatgcggatagcgtgaaaggccgctttaccatcagccgcgataattcgaaaaacaccctgtatctgcaaatgaacagcctgcgtgcggaagatacggccgtgtattattgcgcgcgtgaccgttactctatgatctactcttacggtgctggtgctttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 134 - protein, 9 aa, Artificial Sequence
  source: /note=“Description of Artificial Sequence Synthetic peptide”
QQTWRKPRT

SEQ ID NO: 135 - protein, 6 aa, Artificial Sequence
  source: /note=“Description of Artificial Sequence Synthetic peptide”
TWRKPR

SEQ ID NO: 136 - protein, 107 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQTWRKPRTFGQGTKVEIK

SEQ ID NO: 137 - DNA, 321 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcagacttggcgtaaaccgcgtacctttggccagggcacgaaagttgaaattaaa

SEQ ID NO: 138 - protein, 214 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQTWRKPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - DNA, 642 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcagacttggcgtaaaccgcgtacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 140 - DNA, 375 bp, Artificial Sequence
  source: /note=“Description of Artificial Sequence Synthetic polynucleotide”
caagtgcagctgcttgagagcggtggcggactggtgcagccagggggatccttgcgcctgtcatgcgctgcgtcggggttcaccttctcgtcctactggatgaactgggtcagacaggctccggggaagggactcgaatgggtgtccgccatttcctccgacggctcctacacttactacgccgatagcgtcaagggccggttcaccatctcccgggacaattcgaagaacaccctgtacctccaaatgaactcactgcgcgccgaggacactgcggtgtattactgtgcccgggataggtacagcatgatctactcctacggtgccggagcctttgactactggggacagggaacccttgtgaccgtgtctagc

SEQ ID NO: 141 - protein, 455 aa, Artificial Sequence
  source: /note=“Description of Artificial Sequence Synthetic polypeptide”
QVQLLESGGGLVQPGGSLRLSCAASGFTFSSYWMNWVRQAPGKGLEWVSAISSDGSYTYYADSVKGRFTISRDNSKNTLYLQMNSLRAEDTAVYYCARDRYSMIYSYGAGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - DNA, 1365 bp, Artificial Sequence
  source: /note=“Description of Artificial Sequence Synthetic polynucleotide”
caagtgcagctgcttgagagcggtggcggactggtgcagccagggggatccttgcgcctgtcatgcgctgcgtcggggttcaccttctcgtcctactggatgaactgggtcagacaggctccggggaagggactcgaatgggtgtccgccatttcctccgacggctcctacacttactacgccgatagcgtcaagggccggttcaccatctcccgggacaattcgaagaacaccctgtacctccaaatgaactcactgcgcgccgaggacactgcggtgtattactgtgcccgggataggtacagcatgatctactcctacggtgccggagcctttgactactggggacagggaacccttgtgaccgtgtctagcgcgtccactaagggcccgtcagtgttcccgctggctccatcgtcgaagtccacctccggaggaaccgcagcactcggttgcctggtcaaggactacttccctgagccagtgaccgtgtcgtggaacagcggagccctgacttccggcgtgcacacttttcccgcggtgctgcagtcctccggtctgtactccctttcgtccgtggtcaccgtgccgtcgtctagcctgggcacccagacctacatctgcaacgtgaaccacaagccgtccaacaccaaagtggataagcgggtggagccgaagtcctgcgataagacacacacgtgcccgccatgtccagcgcctgaattgcttggcggaccttccgtgttcctgttcccgcctaagcccaaggacaccttgatgattagccggactcccgaagtcacctgtgtggtggtggcagtgtcccacgaggaccccgaggtcaagtttaattggtacgtggacggcgtcgaagtgcacaacgccaagactaagccccgggaggaacagtacaacagcacctaccgggtcgtgtccgtgctgaccgtgctgcaccaggactggctgaatgggaaagagtacaagtgcaaagtgtccaacaaggccttggccgctcctatcgaaaaaactatcagcaaggctaagggacagccgagggaaccccaagtctacaccctgcccccttcacgcgaagagatgaccaagaatcaagtgtcgctgacctgcctcgtcaagggattctacccctccgacattgcggtggagtgggagtccaacggccagcccgagaacaactacaagactactccgcccgtgctggactccgacggcagcttcttcctgtattccaagctgaccgtggacaagtcccggtggcagcaaggaaacgtgttctcctgctcggtcatgcacgaagccctgcacaaccactatacgcagaagtccctgtccttgagcccggggaaa

SEQ ID NO: 143 - DNA, 321 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaaacctggcggaagcccaggacatttggccagggcactaaggtcgagattaag

SEQ ID NO: 144 - DNA, 642 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaaacctggcggaagcccaggacatttggccagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 145 - protein, 9 aa, Artificial Sequence
  source: /note=“Description of Artificial Sequence Synthetic peptide”
QQIWTVPRT

SEQ ID NO: 146 - protein, 6 aa, Artificial Sequence
  source: /note=“Description of Artificial Sequence Synthetic peptide”
IWTVPR

SEQ ID NO: 147 - protein, 107 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQIWTVPRTFGQGTKVEIK

SEQ ID NO: 148 - DNA, 321 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcagatctggactgttccgcgtacctttggccagggcacgaaagttgaaattaaa

SEQ ID NO: 149 - protein, 214 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQIWTVPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - DNA, 642 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcagatctggactgttccgcgtacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 151 - protein, 17 aa, Artificial Sequence
  source: /note=“Description of Artificial Sequence Synthetic peptide”
SIGGQGGMTLYADSVKG

SEQ ID NO: 152 - protein, 6 aa, Artificial Sequence
  source: /note=“Description of Artificial Sequence Synthetic peptide”
GGQGGM

SEQ ID NO: 153 - protein, 8 aa, Artificial Sequence
  source: /note=“Description of Artificial Sequence Synthetic peptide”
IGGQGGMT

SEQ ID NO: 154 - protein, 121 aa, Artificial Sequence
  source: /note=“Description of Artificial Sequence Synthetic polypeptide”
EVQLLESGGGLVQPGGSLRLSCAASGFTFNTHYIHWVRQAPGKGLEWVSSIGGQGGMTLYADSVKGRFTISRDNSKNTLYLQMNSLRAEDTAVYYCARERGYVYYHMFDPWGQGTLVTVSS

SEQ ID NO: 155 - DNA, 363 bp, Artificial Sequence
  source: /note=“Description of Artificial Sequence Synthetic polynucleotide”
gaggtgcaattgctggaaagcggcggtggcctggtgcagccgggtggcagcctgcgtctgagctgcgcggcgtccggattcacctttaacactcattacatccattgggtgcgccaggcccccggcaaaggtctcgagtgggtttcctctatcggtggtcagggcggtatgactctgtatgcggatagcgtgaaaggccgctttaccatcagccgcgataattcgaaaaacaccctgtatctgcaaatgaacagcctgcgtgcggaagatacggccgtgtattattgcgcgcgtgaacgtggttacgtttactaccatatgttcgatccgtggggccaaggcaccctggtgactgttagctca

SEQ ID NO: 156 - protein, 451 aa, Artificial Sequence
  source: /note=“Description of Artificial Sequence Synthetic polypeptide”
EVQLLESGGGLVQPGGSLRLSCAASGFTFNTHYIHWVRQAPGKGLEWVSSIGGQGGMTLYADSVKGRFTISRDNSKNTLYLQMNSLRAEDTAVYYCARERGYVYYHMFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - DNA, 1353 bp, Artificial Sequence
  source: /note=“Description of Artificial Sequence Synthetic polynucleotide”
gaggtgcaattgctggaaagcggcggtggcctggtgcagccgggtggcagcctgcgtctgagctgcgcggcgtccggattcacctttaacactcattacatccattgggtgcgccaggcccccggcaaaggtctcgagtgggtttcctctatcggtggtcagggcggtatgactctgtatgcggatagcgtgaaaggccgctttaccatcagccgcgataattcgaaaaacaccctgtatctgcaaatgaacagcctgcgtgcggaagatacggccgtgtattattgcgcgcgtgaacgtggttacgtttactaccatatgttcgatccgtggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 158 - DNA, 363 bp, Artificial Sequence
  source: /note=“Description of Artificial Sequence Synthetic polynucleotide”
gaagtgcagctcctggagtcgggtggcggactggtgcagcctggcggatcactgcggctgtcatgtgccgcgagcgggtttactttcaacacccactacatccactgggtccgccaagctcccggaaagggactcgaatgggtgtcctccattggtggacagggcggcatgaccctttacgcggatagcgtgaaggggaggttcaccatctcccgcgacaacagcaagaacaccctgtacctccaaatgaactcgcttcgggccgaggacactgccgtgtactattgcgcaagagagcggggctacgtgtactaccacatgttcgacccatggggacagggaacgctggtcaccgtgtcctcc

SEQ ID NO: 159 - protein, 451 aa, Artificial Sequence
  source: /note=“Description of Artificial Sequence Synthetic polypeptide”
EVQLLESGGGLVQPGGSLRLSCAASGFTFNTHYIHWVRQAPGKGLEWVSSIGGQGGMTLYADSVKGRFTISRDNSKNTLYLQMNSLRAEDTAVYYCARERGYVYYHMFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - DNA, 1353 bp, Artificial Sequence
  source: /note=“Description of Artificial Sequence Synthetic polynucleotide”
gaagtgcagctcctggagtcgggtggcggactggtgcagcctggcggatcactgcggctgtcatgtgccgcgagcgggtttactttcaacacccactacatccactgggtccgccaagctcccggaaagggactcgaatgggtgtcctccattggtggacagggcggcatgaccctttacgcggatagcgtgaaggggaggttcaccatctcccgcgacaacagcaagaacaccctgtacctccaaatgaactcgcttcgggccgaggacactgccgtgtactattgcgcaagagagcggggctacgtgtactaccacatgttcgacccatggggacagggaacgctggtcaccgtgtcctccgcctccactaagggcccgtcagtgttcccgctggctccatcgtcgaagtccacctccggaggaaccgcagcactcggttgcctggtcaaggactacttccctgagccagtgaccgtgtcgtggaacagcggagccctgacttccggcgtgcacacttttcccgcggtgctgcagtcctccggtctgtactccctttcgtccgtggtcaccgtgccgtcgtctagcctgggcacccagacctacatctgcaacgtgaaccacaagccgtccaacaccaaagtggataagcgggtggagccgaagtcctgcgataagacacacacgtgcccgccatgtccagcgcctgaattgcttggcggaccttccgtgttcctgttcccgcctaagcccaaggacaccttgatgattagccggactcccgaagtcacctgtgtggtggtggcagtgtcccacgaggaccccgaggtcaagtttaattggtacgtggacggcgtcgaagtgcacaacgccaagactaagccccgggaggaacagtacaacagcacctaccgggtcgtgtccgtgctgaccgtgctgcaccaggactggctgaatgggaaagagtacaagtgcaaagtgtccaacaaggccttggccgctcctatcgaaaaaactatcagcaaggctaagggacagccgagggaaccccaagtctacaccctgcccccttcacgcgaagagatgaccaagaatcaagtgtcgctgacctgcctcgtcaagggattctacccctccgacattgcggtggagtgggagtccaacggccagcccgagaacaactacaagactactccgcccgtgctggactccgacggcagcttcttcctgtattccaagctgaccgtggacaagtcccggtggcagcaaggaaacgtgttctcctgctcggtcatgcacgaagccctgcacaaccactatacgcagaagtccctgtccttgagcccggggaaa

SEQ ID NO: 161 - protein, 121 aa, Artificial Sequence
  source: /note=“Description of Artificial Sequence Synthetic polypeptide”
EVQLLESGGGLVQPGGSLRLSCAASGFTFSTHYIHWVRQAPGKGLEWVSSIGGQGGMTLYADSVKGRFTISRDNSKNTLYLQMNSLRAEDTAVYYCARERGYVYYHMFDPWGQGTLVTVSS

SEQ ID NO: 162 - DNA, 363 bp, Artificial Sequence
  source: /note=“Description of Artificial Sequence Synthetic polynucleotide”
gaagtgcagctcctggagtcgggtggcggactggtgcagcctggcggatcactgcggctgtcatgtgccgcgagcgggtttactttctccacccactacatccactgggtccgccaagctcccggaaagggactcgaatgggtgtcctccattggtggacagggcggcatgaccctttacgcggatagcgtgaaggggaggttcaccatctcccgcgacaacagcaagaacaccctgtacctccaaatgaactcgcttcgggccgaggacactgccgtgtactattgcgcaagagagcggggctacgtgtactaccacatgttcgacccatggggacagggaacgctggtcaccgtgtcctcc

SEQ ID NO: 163 - protein, 451 aa, Artificial Sequence
  source: /note=“Description of Artificial Sequence Synthetic polypeptide”
EVQLLESGGGLVQPGGSLRLSCAASGFTFSTHYIHWVRQAPGKGLEWVSSIGGQGGMTLYADSVKGRFTISRDNSKNTLYLQMNSLRAEDTAVYYCARERGYVYYHMFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - DNA, 1353 bp, Artificial Sequence
  source: /note=“Description of Artificial Sequence Synthetic polynucleotide”
gaagtgcagctcctggagtcgggtggcggactggtgcagcctggcggatcactgcggctgtcatgtgccgcgagcgggtttactttctccacccactacatccactgggtccgccaagctcccggaaagggactcgaatgggtgtcctccattggtggacagggcggcatgaccctttacgcggatagcgtgaaggggaggttcaccatctcccgcgacaacagcaagaacaccctgtacctccaaatgaactcgcttcgggccgaggacactgccgtgtactattgcgcaagagagcggggctacgtgtactaccacatgttcgacccatggggacagggaacgctggtcaccgtgtcctccgcctccactaagggcccgtcagtgttcccgctggctccatcgtcgaagtccacctccggaggaaccgcagcactcggttgcctggtcaaggactacttccctgagccagtgaccgtgtcgtggaacagcggagccctgacttccggcgtgcacacttttcccgcggtgctgcagtcctccggtctgtactccctttcgtccgtggtcaccgtgccgtcgtctagcctgggcacccagacctacatctgcaacgtgaaccacaagccgtccaacaccaaagtggataagcgggtggagccgaagtcctgcgataagacacacacgtgcccgccatgtccagcgcctgaattgcttggcggaccttccgtgttcctgttcccgcctaagcccaaggacaccttgatgattagccggactcccgaagtcacctgtgtggtggtggcagtgtcccacgaggaccccgaggtcaagtttaattggtacgtggacggcgtcgaagtgcacaacgccaagactaagccccgggaggaacagtacaacagcacctaccgggtcgtgtccgtgctgaccgtgctgcaccaggactggctgaatgggaaagagtacaagtgcaaagtgtccaacaaggccttggccgctcctatcgaaaaaactatcagcaaggctaagggacagccgagggaaccccaagtctacaccctgcccccttcacgcgaagagatgaccaagaatcaagtgtcgctgacctgcctcgtcaagggattctacccctccgacattgcggtggagtgggagtccaacggccagcccgagaacaactacaagactactccgcccgtgctggactccgacggcagcttcttcctgtattccaagctgaccgtggacaagtcccggtggcagcaaggaaacgtgttctcctgctcggtcatgcacgaagccctgcacaaccactatacgcagaagtccctgtccttgagcccggggaaa

SEQ ID NO: 165 - protein, 10 aa, Artificial Sequence
  source: /note=“Description of Artificial Sequence Synthetic peptide”
GFTFQTHYIH

SEQ ID NO: 166 - protein, 7 aa, Artificial Sequence
  source: /note=“Description of Artificial Sequence Synthetic peptide”
GFTFQTH

SEQ ID NO: 167 - protein, 8 aa, Artificial Sequence
  source: /note=“Description of Artificial Sequence Synthetic peptide”
GFTFQTHY

SEQ ID NO: 168 - protein, 121 aa, Artificial Sequence
  source: /note=“Description of Artificial Sequence Synthetic polypeptide”
EVQLLESGGGLVQPGGSLRLSCAASGFTFQTHYIHWVRQAPGKGLEWVSSIGGQGGMTLYADSVKGRFTISRDNSKNTLYLQMNSLRAEDTAVYYCARERGYVYYHMFDPWGQGTLVTVSS

SEQ ID NO: 169 - DNA, 363 bp, Artificial Sequence
  source: /note=“Description of Artificial Sequence Synthetic polynucleotide”
gaagtgcagctcctggagtcgggtggcggactggtgcagcctggcggatcactgcggctgtcatgtgccgcgagcgggtttactttccagacccactacatccactgggtccgccaagctcccggaaagggactcgaatgggtgtcctccattggtggacagggcggcatgaccctttacgcggatagcgtgaaggggaggttcaccatctcccgcgacaacagcaagaacaccctgtacctccaaatgaactcgcttcgggccgaggacactgccgtgtactattgcgcaagagagcggggctacgtgtactaccacatgttcgacccatggggacagggaacgctggtcaccgtgtcctcc

SEQ ID NO: 170 - protein, 451 aa, Artificial Sequence
  source: /note=“Description of Artificial Sequence Synthetic polypeptide”
EVQLLESGGGLVQPGGSLRLSCAASGFTFQTHYIHWVRQAPGKGLEWVSSIGGQGGMTLYADSVKGRFTISRDNSKNTLYLQMNSLRAEDTAVYYCARERGYVYYHMFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - DNA, 1353 bp, Artificial Sequence
  source: /note=“Description of Artificial Sequence Synthetic polynucleotide”
gaagtgcagctcctggagtcgggtggcggactggtgcagcctggcggatcactgcggctgtcatgtgccgcgagcgggtttactttccagacccactacatccactgggtccgccaagctcccggaaagggactcgaatgggtgtcctccattggtggacagggcggcatgaccctttacgcggatagcgtgaaggggaggttcaccatctcccgcgacaacagcaagaacaccctgtacctccaaatgaactcgcttcgggccgaggacactgccgtgtactattgcgcaagagagcggggctacgtgtactaccacatgttcgacccatggggacagggaacgctggtcaccgtgtcctccgcctccactaagggcccgtcagtgttcccgctggctccatcgtcgaagtccacctccggaggaaccgcagcactcggttgcctggtcaaggactacttccctgagccagtgaccgtgtcgtggaacagcggagccctgacttccggcgtgcacacttttcccgcggtgctgcagtcctccggtctgtactccctttcgtccgtggtcaccgtgccgtcgtctagcctgggcacccagacctacatctgcaacgtgaaccacaagccgtccaacaccaaagtggataagcgggtggagccgaagtcctgcgataagacacacacgtgcccgccatgtccagcgcctgaattgcttggcggaccttccgtgttcctgttcccgcctaagcccaaggacaccttgatgattagccggactcccgaagtcacctgtgtggtggtggcagtgtcccacgaggaccccgaggtcaagtttaattggtacgtggacggcgtcgaagtgcacaacgccaagactaagccccgggaggaacagtacaacagcacctaccgggtcgtgtccgtgctgaccgtgctgcaccaggactggctgaatgggaaagagtacaagtgcaaagtgtccaacaaggccttggccgctcctatcgaaaaaactatcagcaaggctaagggacagccgagggaaccccaagtctacaccctgcccccttcacgcgaagagatgaccaagaatcaagtgtcgctgacctgcctcgtcaagggattctacccctccgacattgcggtggagtgggagtccaacggccagcccgagaacaactacaagactactccgcccgtgctggactccgacggcagcttcttcctgtattccaagctgaccgtggacaagtcccggtggcagcaaggaaacgtgttctcctgctcggtcatgcacgaagccctgcacaaccactatacgcagaagtccctgtccttgagcccggggaaa

SEQ ID NO: 172 - protein, 9 aa, Artificial Sequence
  source: /note=“Description of Artificial Sequence Synthetic peptide”
QQEWAKPRT

SEQ ID NO: 173 - protein, 6 aa, Artificial Sequence
  source: /note=“Description of Artificial Sequence Synthetic peptide”
EWAKPR

SEQ ID NO: 174 - protein, 107 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQEWAKPRTFGQGTKVEIK

SEQ ID NO: 175 - DNA, 321 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcaggaatgggctaaaccgcgtacctttggccagggcacgaaagttgaaattaaa

SEQ ID NO: 176 - protein, 214 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQEWAKPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - DNA, 642 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcaggaatgggctaaaccgcgtacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 178 - protein, 9 aa, Artificial Sequence
  source: /note=“Description of Artificial Sequence Synthetic peptide”
QQSWTRPRT

SEQ ID NO: 179 - protein, 6 aa, Artificial Sequence
  source: /note=“Description of Artificial Sequence Synthetic peptide”
SWTRPR

SEQ ID NO: 180 - protein, 107 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQSWTRPRTFGQGTKVEIK

SEQ ID NO: 181 - DNA, 321 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcagtcttggactcgtccgcgtacctttggccagggcacgaaagttgaaattaaa

SEQ ID NO: 182 - protein, 214 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQSWTRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - DNA, 642 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcagtcttggactcgtccgcgtacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 184 - protein, 9 aa, Artificial Sequence
  source: /note=“Description of Artificial Sequence Synthetic peptide”
QQIWMAPRT

SEQ ID NO: 185 - protein, 6 aa, Artificial Sequence
  source: /note=“Description of Artificial Sequence Synthetic peptide”
IWMAPR

SEQ ID NO: 186 - protein, 107 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQIWMAPRTFGQGTKVEIK

SEQ ID NO: 187 - DNA, 321 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcagatctggatggctccgcgtacctttggccagggcacgaaagttgaaattaaa

SEQ ID NO: 188 - protein, 214 aa, Artificial Sequence
  source: /note=“Description of Artificial Sequence Synthetic polypeptide”
DIQMTQSPSSLSASVGDRVTITCRASQGISSYLAWYQQKPGKAPKLLIYTASTLQSGVPSRFSGSGSGTDFTLTISSLQPEDFATYYCQQIWMAPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - DNA, 642 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagggtatttcttcttacctggcttggtaccagcagaaaccgggcaaagcgccgaaactattaatctacactgcttctactctgcaaagcggcgtgccgagccgctttagcggcagcggatccggcaccgatttcaccctgaccattagctctctgcaaccggaagactttgcgacctattattgccagcagatctggatggctccgcgtacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 190 - protein, 17 aa, Artificial Sequence
  source: /note=“Description of Artificial Sequence Synthetic peptide”
AISSKGSYTYYADSVKG

SEQ ID NO: 191 - protein, 6 aa, Artificial Sequence
  source: /note=“Description of Artificial Sequence Synthetic peptide”
SSKGSY

SEQ ID NO: 192 - protein, 8 aa, Artificial Sequence
  source: /note=“Description of Artificial Sequence Synthetic peptide”
ISSKGSYT

SEQ ID NO: 193 - protein, 125 aa, Artificial Sequence
  source: /note=“Description of Artificial Sequence Synthetic polypeptide”
QVQLLESGGGLVQPGGSLRLSCAASGFTFSSYWMNWVRQAPGKGLEWVSAISSKGSYTYYADSVKGRFTISRDNSKNTLYLQMNSLRAEDTAVYYCARDRYSMIYSYGAGAFDYWGQGTLVTVSS

SEQ ID NO: 194 - DNA, 375 bp, Artificial Sequence
  source: /note=“Description of Artificial Sequence Synthetic polynucleotide”
caagttcagctccttgagtctggggggggcctggtgcaacctgggggctctctgcggctttcatgtgcggcctcagggttcactttcagctcatactggatgaattgggtacgccaagctccaggcaaaggactcgaatgggtaagcgctatatccagcaaagggagctatacctattacgcggattccgttaagggcaggttcactatatcccgcgacaactccaaaaatactttgtatctgcaaatgaattccctccgagccgaagataccgcagtatattactgtgcgagggacaggtactccatgatttacagctacggtgccggtgctttcgattattggggacaggggacacttgtgaccgtcagttct

SEQ ID NO: 195 - protein, 455 aa, Artificial Sequence
  source: /note=“Description of Artificial Sequence Synthetic polypeptide”
QVQLLESGGGLVQPGGSLRLSCAASGFTFSSYWMNWVRQAPGKGLEWVSAISSKGSYTYYADSVKGRFTISRDNSKNTLYLQMNSLRAEDTAVYYCARDRYSMIYSYGAGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - DNA, 1365 bp, Artificial Sequence
  source: /note=“Description of Artificial Sequence Synthetic polynucleotide”
caagttcagctccttgagtctggggggggcctggtgcaacctgggggctctctgcggctttcatgtgcggcctcagggttcactttcagctcatactggatgaattgggtacgccaagctccaggcaaaggactcgaatgggtaagcgctatatccagcaaagggagctatacctattacgcggattccgttaagggcaggttcactatatcccgcgacaactccaaaaatactttgtatctgcaaatgaattccctccgagccgaagataccgcagtatattactgtgcgagggacaggtactccatgatttacagctacggtgccggtgctttcgattattggggacaggggacacttgtgaccgtcagttctgcaagtaccaaagggccgtctgttttcccattggctccctcatccaagagcacgagtggaggcaccgccgcgctgggatgccttgtgaaagactatttcccggagcccgtgaccgttagctggaacagcggcgctcttaccagtggcgttcacacattcccagctgttttgcagtcatccgggctctactctctctcatccgtggtcaccgtgccgtctagttctttgggcacccagacctacatctgtaacgtaaatcacaaacctagtaatactaaggtggacaagcgagttgaaccgaagagctgtgataagacacatacttgtccaccatgtccggcacccgaggcagcgggggggcccagtgtttttctcttcccacccaagcccaaagacacattgatgatctcacgaaccccagaggtaacttgtgtcgtggtagatgtaagccatgaggaccccgaagttaagttcaattggtatgttgacggtgtagaggtgcacaatgccaaaactaaaccccgggaggagcaatacaactcaacttacagagtcgtatccgtgctgaccgttttgcaccaggattggttgaatggtaaggaatacaaatgtaaagtgagcaataaagctctcccagcgcccatcgagaagaccattagcaaagccaagggtcaacccagggaaccccaggtatatacgctgccaccctcaagggaagagatgacaaagaatcaagtgtcactgacgtgtcttgtcaagggtttctatcctagcgacattgcggtggaatgggagtcaaatgggcaacccgagaacaactacaagactactcctcccgtcctggacagcgacggctccttcttcctgtatagtaaactgaccgtcgataaaagtaggtggcagcaggggaatgtctttagttgctctgtcatgcatgaggcgctccataaccactacacccaaaaatctttgagcttgagccctgggaaa

SEQ ID NO: 197 - DNA, 321 bp, Artificial Sequence
  source: /note=“Description of Artificial Sequence Synthetic polynucleotide”
gacattcaaatgacacaaagtccgtccagtcttagtgcttctgtgggcgatagggtcaccatcacttgtcgggcgtctcaggggatcagctcttacttggcatggtatcaacaaaagccaggaaaagcacctaaattgcttatttatacagcgtccaccctccagtcaggagtgcctagtaggttctcaggctctgggtccggtactgacttcacgctgactatatcaagcttgcaacccgaagattttgcaacatactactgccaacagacatggaggaagccaagaactttcggtcagggaacgaaagttgagataaag

SEQ ID NO: 198 - DNA, 642 bp, Artificial Sequence
  source: /note=“Description of Artificial Sequence Synthetic polynucleotide”
gacattcaaatgacacaaagtccgtccagtcttagtgcttctgtgggcgatagggtcaccatcacttgtcgggcgtctcaggggatcagctcttacttggcatggtatcaacaaaagccaggaaaagcacctaaattgcttatttatacagcgtccaccctccagtcaggagtgcctagtaggttctcaggctctgggtccggtactgacttcacgctgactatatcaagcttgcaacccgaagattttgcaacatactactgccaacagacatggaggaagccaagaactttcggtcagggaacgaaagttgagataaagcgcactgtcgcagcaccttccgtgttcattttcccgccttccgacgagcagcttaaatcagggaccgcgagtgttgtttgcttgcttaataacttttacccacgggaagccaaagttcagtggaaggtggacaatgcactccaaagcgggaatagtcaggagtcagttactgagcaagatagtaaagactctacttactctttgagttcaaccttgaccctctcaaaagcggactacgagaagcataaagtgtacgcctgcgaggtgacgcatcaaggtttgtcttccccggttacgaagtcctttaataggggggaatgt

SEQ ID NO: 199 - DNA, 321 bp, Artificial Sequence
  source: /note=“Description of Artificial Sequence Synthetic polynucleotide”
gatatacagatgacgcaaagtccctctagtctttctgcaagtgtcggggacagagttaccattacctgcagagcgtcacaaggcatctctagttatctcgcgtggtaccaacagaagccaggtaaagcacctaaactgttgatttacacggcatcaacattgcagtcaggtgtcccctcccgatttagtggcagtggtagcggtacagattttactcttaccatttcatctcttcagccagaagattttgctacgtactactgtcaacaagaatgggctaaaccacgaacctttggacagggtacgaaggtcgaaataaaa

SEQ ID NO: 200 - DNA, 642 bp, Artificial Sequence
  source: /note=“Description of Artificial Sequence Synthetic polynucleotide”
gatatacagatgacgcaaagtccctctagtctttctgcaagtgtcggggacagagttaccattacctgcagagcgtcacaaggcatctctagttatctcgcgtggtaccaacagaagccaggtaaagcacctaaactgttgatttacacggcatcaacattgcagtcaggtgtcccctcccgatttagtggcagtggtagcggtacagattttactcttaccatttcatctcttcagccagaagattttgctacgtactactgtcaacaagaatgggctaaaccacgaacctttggacagggtacgaaggtcgaaataaaacggaccgttgccgccccctccgtcttcatcttccccccgtctgacgagcagctcaaatccggcacagcttctgtagtctgcttgctgaataacttctacccaagagaagccaaagttcagtggaaggtcgataatgcattgcaatctggtaatagtcaggaatctgtgactgagcaggatagcaaagactcaacttacagcctctcttcaaccttgacgttgtccaaagcggattatgagaaacacaaggtgtacgcttgcgaggtgacgcatcaagggcttagttccccggtaaccaaatctttcaaccgaggtgaatgc

SEQ ID NO: 201 - protein, 125 aa, Artificial Sequence
  source: /note=“Description of Artificial Sequence Synthetic polypeptide”
QVQLLESGGGLVQPGGSLRLSCAASGFTFSSYWMNWVRQAPGKGLEWVSVIESKGNYIFYADSVKGRFTISRDNSKNTLYLQMNSLRAEDTAVYYCARDRYSMIYSYGAGAFDYWGQGTLVTVSS

SEQ ID NO: 202 - DNA, 375 bp, Artificial Sequence
  source: /note=“Description of Artificial Sequence Synthetic polynucleotide”
caagttcaattgctggaaagcggaggtggacttgtccaacctggagggtcactccgactgtcttgcgctgcatcaggattcacctttagtagctattggatgaactgggtccggcaggctcctgggaaagggcttgagtgggtaagtgtcattgaatcaaagggcaactacatcttttatgctgattctgtaaagggtaggttcaccatctccagggacaattcaaaaaatactttgtatctgcagatgaactctctcagggcagaagacacggccgtttattactgcgcccgcgatcgatacagcatgatatactcctacggcgcaggagcttttgactactggggtcaaggcacacttgttactgtcagtagc

SEQ ID NO: 203 - protein, 455 aa, Artificial Sequence
  source: /note=“Description of Artificial Sequence Synthetic polypeptide”
QVQLLESGGGLVQPGGSLRLSCAASGFTFSSYWMNWVRQAPGKGLEWVSVIESKGNYIFYADSVKGRFTISRDNSKNTLYLQMNSLRAEDTAVYYCARDRYSMIYSYGAGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - DNA, 1365 bp, Artificial Sequence
  source: /note=“Description of Artificial Sequence Synthetic polynucleotide”
caagttcaattgctggaaagcggaggtggacttgtccaacctggagggtcactccgactgtcttgcgctgcatcaggattcacctttagtagctattggatgaactgggtccggcaggctcctgggaaagggcttgagtgggtaagtgtcattgaatcaaagggcaactacatcttttatgctgattctgtaaagggtaggttcaccatctccagggacaattcaaaaaatactttgtatctgcagatgaactctctcagggcagaagacacggccgtttattactgcgcccgcgatcgatacagcatgatatactcctacggcgcaggagcttttgactactggggtcaaggcacacttgttactgtcagtagcgcctcaacgaaaggaccgtccgtgtttcctcttgctcctagctccaaatccacctcaggtggaacggccgccctggggtgcctggtaaaggactatttcccagagccagttactgtgtcttggaattctggtgcattgacaagtggcgtacacacttttcccgcggtcctccaatctagtggtctgtactcactgtcctccgttgtgactgtcccaagtagctcacttggcacacagacttacatctgtaatgttaatcataagccgtcaaacacgaaggtggataagagggtagaacctaagtcatgtgacaaaacgcatacttgccccccctgccctgcgccggaagccgctggcggaccctccgtattcttgttccctccaaagccaaaggacactctgatgattagccggacaccggaggtcacttgtgttgtagttgacgtcagccatgaggatcctgaggtgaaatttaattggtacgtggacggggttgaagtccacaatgctaaaactaaacctagggaagagcaatataatagtacatacagggttgtcagtgtgctgaccgttctccatcaggactggctgaacggcaaggaatacaagtgcaaggtcagcaacaaggccttgccggcccccatcgagaagacgatctccaaagccaaggggcaaccccgagaaccgcaggtatacacgctcccccctagtagagaagagatgacaaagaatcaagtttccttgacgtgccttgtgaaaggcttctaccctagtgacatcgcagtcgaatgggagagcaacgggcagccggagaataactataaaacaaccccccccgtgcttgactcagacgggtcattttttctgtatagcaaattgactgttgataaatcacggtggcaacaaggaaacgtgtttagttgcagcgtaatgcacgaagctctccacaatcactatactcaaaagtcactgtcactctcccctggcaag

SEQ ID NO: 205 - DNA, 321 bp, Artificial Sequence
  source: /note=“Description of Artificial Sequence Synthetic polynucleotide”
gacatacaaatgacgcaatctccgagtagcttgtcagcgtccgtaggcgaccgagtaacgattacgtgtagagcgagccagggaatttcatcttatttggcttggtatcagcaaaagccgggaaaagcacccaaactcctcatttatactgccagcacgttgcaaagcggcgttccgagtcggttctctggatcagggtccgggacggacttcaccttgacgatttcatctttgcaacctgaagattttgcaacatactactgtcaacaggagtgggtgaagccaaggaccttcggacaaggcacgaaggtcgaaatcaag

SEQ ID NO: 206 - DNA, 642 bp, Artificial Sequence
  source: /note=“Description of Artificial Sequence Synthetic polynucleotide”
gacatacaaatgacgcaatctccgagtagcttgtcagcgtccgtaggcgaccgagtaacgattacgtgtagagcgagccagggaatttcatcttatttggcttggtatcagcaaaagccgggaaaagcacccaaactcctcatttatactgccagcacgttgcaaagcggcgttccgagtcggttctctggatcagggtccgggacggacttcaccttgacgatttcatctttgcaacctgaagattttgcaacatactactgtcaacaggagtgggtgaagccaaggaccttcggacaaggcacgaaggtcgaaatcaagcgaaccgtggcagctccgtccgtgtttatttttccgccttccgacgaacaacttaaaagtggaacagcctctgtcgtctgtctccttaacaacttctaccccagggaagctaaagtacagtggaaggtagataacgctctgcaaagtggtaattctcaggagagcgtcacggaacaggactccaaagactccacctattctctgagctctacactgacgctcagcaaggcagactacgaaaagcacaaagtatatgcgtgtgaggtgacgcatcaaggccttagcagtccagttacaaaaagttttaacaggggagaatgc

SEQ ID NO: 207 - DNA, 375 bp, Artificial Sequence
  source: /note=“Description of Artificial Sequence Synthetic polynucleotide”
gaagtgcagctgctggaatccggcggaggtctggtccagcctggaggttccctgcgcctgtcatgcgcagcctccggattcaccttttcgtcgtactggatgaactgggtcagacaggctcctggaaagggcctggaatgggtgtctgtgattgaatccaaggggaactacatcttctacgcggacagcgtgaagggccggttcactatcagcagagacaacagcaagaacaccctgtacctccaaatgaactcgctgagggccgaagatactgccgtgtactactgtgcccgcgatcgctactcgatgatctacagctatggtgccggagcgttcgattactggggacagggaaccctcgtgaccgtcagctcc

SEQ ID NO: 208 - protein, 455 aa, Artificial Sequence
  source: /note=“Description of Artificial Sequence Synthetic polypeptide”
EVQLLESGGGLVQPGGSLRLSCAASGFTFSSYWMNWVRQAPGKGLEWVSVIESKGNYIFYADSVKGRFTISRDNSKNTLYLQMNSLRAEDTAVYYCARDRYSMIYSYGAGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - DNA, 1365 bp, Artificial Sequence
  source: /note=“Description of Artificial Sequence Synthetic polynucleotide”
gaagtgcagctgctggaatccggcggaggtctggtccagcctggaggttccctgcgcctgtcatgcgcagcctccggattcaccttttcgtcgtactggatgaactgggtcagacaggctcctggaaagggcctggaatgggtgtctgtgattgaatccaaggggaactacatcttctacgcggacagcgtgaagggccggttcactatcagcagagacaacagcaagaacaccctgtacctccaaatgaactcgctgagggccgaagatactgccgtgtactactgtgcccgcgatcgctactcgatgatctacagctatggtgccggagcgttcgattactggggacagggaaccctcgtgaccgtcagctccgcctcaaccaagggcccgtcagtgttcccgctggctccatcgtcgaagtccacctccggaggaaccgcagcactcggttgcctggtcaaggactacttccctgagccagtgaccgtgtcgtggaacagcggagccctgacttccggcgtgcacacttttcccgcggtgctgcagtcctccggtctgtactccctttcgtccgtggtcaccgtgccgtcgtctagcctgggcacccagacctacatctgcaacgtgaaccacaagccgtccaacaccaaagtggataagcgggtggagccgaagtcctgcgataagacacacacgtgcccgccatgtccagcgcctgaattgcttggcggaccttccgtgttcctgttcccgcctaagcccaaggacaccttgatgattagccggactcccgaagtcacctgtgtggtggtggcagtgtcccacgaggaccccgaggtcaagtttaattggtacgtggacggcgtcgaagtgcacaacgccaagactaagccccgggaggaacagtacaacagcacctaccgggtcgtgtccgtgctgaccgtgctgcaccaggactggctgaatgggaaagagtacaagtgcaaagtgtccaacaaggccttggccgctcctatcgaaaaaactatcagcaaggctaagggacagccgagggaaccccaagtctacaccctgcccccttcacgcgaagagatgaccaagaatcaagtgtcgctgacctgcctcgtcaagggattctacccctccgacattgcggtggagtgggagtccaacggccagcccgagaacaactacaagactactccgcccgtgctggactccgacggcagcttcttcctgtattccaagctgaccgtggacaagtcccggtggcagcaaggaaacgtgttctcctgctcggtcatgcacgaagccctgcacaaccactatacgcagaagtccctgtccttgagcccggggaaa

SEQ ID NO: 210 - DNA, 321 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaagaatgggtgaagcccaggacatttggccagggcactaaggtcgagattaag

SEQ ID NO: 211 - DNA, 642 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaagaatgggtgaagcccaggacatttggccagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 212 - DNA, 321 bp, Artificial Sequence
  source: /note=“Description of Artificial Sequence Synthetic polynucleotide”
gacatacagatgactcagagtccttcctccctcagtgcttcagtgggtgatcgcgtgacgatcacgtgcagagcctcacaagggatctccagttacctggcctggtatcaacaaaaaccaggcaaggcgcctaagctgttgatatatacggcatctacattgcagtctggggtaccaagtcgattcagtggttctggctcaggcactgactttacccttacaatatcaagtcttcagccggaggatttcgcaacttactattgccagcagatttggacggtgccgcgcactttcggtcagggaacaaaggtggaaataaaa

SEQ ID NO: 213 - DNA, 642 bp, Artificial Sequence
  source: /note=“Description of Artificial Sequence Synthetic polynucleotide”
gacatacagatgactcagagtccttcctccctcagtgcttcagtgggtgatcgcgtgacgatcacgtgcagagcctcacaagggatctccagttacctggcctggtatcaacaaaaaccaggcaaggcgcctaagctgttgatatatacggcatctacattgcagtctggggtaccaagtcgattcagtggttctggctcaggcactgactttacccttacaatatcaagtcttcagccggaggatttcgcaacttactattgccagcagatttggacggtgccgcgcactttcggtcagggaacaaaggtggaaataaaaagaacggtcgcagcaccgagtgttttcatcttccctccctccgacgagcagcttaaaagcggtacagccagcgtagtgtgtttgttgaataatttttatccacgcgaagcaaaagttcagtggaaggtagacaacgcattgcaaagcggaaattcccaagaaagtgttacggagcaagacagtaaggactctacatattccttgtcatcaacactcacccttagtaaagcagattacgagaaacacaaggtctatgcatgtgaggtaacgcatcagggcctctccagtcccgtcaccaagtccttcaacaggggtgagtgc

SEQ ID NO: 214 - DNA, 321 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaaatctggaccgtgcccaggacatttggccagggcactaaggtcgagattaag

SEQ ID NO: 215 - DNA, 642 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaaatctggaccgtgcccaggacatttggccagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 216 - DNA, 321 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaagaatgggccaagcccaggacatttggccagggcactaaggtcgagattaag

SEQ ID NO: 217 - DNA, 642 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaagaatgggccaagcccaggacatttggccagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 218 - DNA, 321 bp, Artificial Sequence
  source: /note=“Description of Artificial Sequence Synthetic polynucleotide”
gatattcagatgacgcaatctccgtcttccttgtcagctagtgtaggagaccgcgtcacaattacctgtagagccagccaggggatttcctcataccttgcatggtaccagcaaaagccaggcaaagcccccaaactgctgatctacaccgcgtctaccttgcaatctggtgtgccgtcacgcttttccggctctggctcaggtactgatttcacattgacgatctcaagtctccagccggaagacttcgcaacttactactgccaacaatcctggacgaggccgaggactttcgggcagggaacaaaggttgaaattaaa

SEQ ID NO: 219 - DNA, 642 bp, Artificial Sequence
  source: /note=“Description of Artificial Sequence Synthetic polynucleotide”
gatattcagatgacgcaatctccgtcttccttgtcagctagtgtaggagaccgcgtcacaattacctgtagagccagccaggggatttcctcataccttgcatggtaccagcaaaagccaggcaaagcccccaaactgctgatctacaccgcgtctaccttgcaatctggtgtgccgtcacgcttttccggctctggctcaggtactgatttcacattgacgatctcaagtctccagccggaagacttcgcaacttactactgccaacaatcctggacgaggccgaggactttcgggcagggaacaaaggttgaaattaaaagaacagtcgcagcaccaagtgtttttatttttccaccctcagacgagcagctcaagtctggcaccgcgagcgtagtatgtttgttgaataatttttaccctagggaagctaaggtacagtggaaagtggataatgctctccaaagtggcaactcccaggaatcagtgactgagcaagattcaaaggacagcacgtattctctttcttctacgcttactctctctaaggccgactacgaaaaacacaaagtttacgcttgcgaggttacccaccaggggctgtcctcaccagtaacgaaaagttttaaccggggcgagtgt

SEQ ID NO: 220 - DNA, 321 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaaagctggaccaggcccaggacatttggccagggcactaaggtcgagattaag

SEQ ID NO: 221 - DNA, 642 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaaagctggaccaggcccaggacatttggccagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 222 - DNA, 321 bp, Artificial Sequence
  source: /note=“Description of Artificial Sequence Synthetic polynucleotide”
gacattcaaatgactcagtctccctcatctttgtcagcatcagttggggacagggtgacaatcacatgccgagcctcacaggggatttctagctatcttgcatggtaccaacagaagcccggcaaagcccccaagcttttgatatatacggcatccactcttcagagcggagtacccagtaggtttagtggctccgggagtggtacggactttactctgacgatttcctcccttcaacctgaagactttgcaacgtattactgtcagcaaatatggatggctcccagaacgtttggtcaaggtactaaagttgaaataaag

SEQ ID NO: 223 - DNA, 642 bp, Artificial Sequence
  source: /note=“Description of Artificial Sequence Synthetic polynucleotide”
gacattcaaatgactcagtctccctcatctttgtcagcatcagttggggacagggtgacaatcacatgccgagcctcacaggggatttctagctatcttgcatggtaccaacagaagcccggcaaagcccccaagcttttgatatatacggcatccactcttcagagcggagtacccagtaggtttagtggctccgggagtggtacggactttactctgacgatttcctcccttcaacctgaagactttgcaacgtattactgtcagcaaatatggatggctcccagaacgtttggtcaaggtactaaagttgaaataaagcgaactgtagcagcacctagtgtatttatcttccccccttctgatgaacagttgaagtccgggacggcttccgtcgtatgtctcctgaacaacttttacccaagggaggcaaaggtgcaatggaaggtggataatgcactccagagtggcaatagccaagaatcagtaaccgaacaggattccaaggattctacctacagcctttcctctacgcttacattgagcaaggcggactatgaaaagcataaggtgtatgcgtgcgaagtaacacaccagggtctcagcagtccagttacgaagtctttcaatcggggagaatgt

SEQ ID NO: 224 - DNA, 321 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaaatctggatggcccccaggacatttggccagggcactaaggtcgagattaag

SEQ ID NO: 225 - DNA, 642 bp, Artificial Sequence
  source: /note=“Description of Artificial Sequence Synthetic polynucleotide”
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaaatctggatggcccccaggacatttggccagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 226 - protein, 10 aa, Artificial Sequence
  source: /note=“Description of Artificial Sequence Synthetic peptide”
GFTFSSYWIS

SEQ ID NO: 227 - protein, 17 aa, Artificial Sequence
  source: /note=“Description of Artificial Sequence Synthetic peptide”
NIKQSGSETYYVESVKG

SEQ ID NO: 228 - protein, 14 aa, Artificial Sequence
  source: /note=“Description of Artificial Sequence Synthetic peptide”
SLRRRSTEHAGFDV

SEQ ID NO: 229 - protein, 5 aa, Artificial Sequence
  source: /note=“Description of Artificial Sequence Synthetic peptide”
SYWIS

SEQ ID NO: 230 - protein, 6 aa, Artificial Sequence
  source: /note=“Description of Artificial Sequence Synthetic peptide”
KQSGSE

SEQ ID NO: 231 - protein, 8 aa, Artificial Sequence
  source: /note=“Description of Artificial Sequence Synthetic peptide”
IKQSGSET

SEQ ID NO: 232 - protein, 16 aa, Artificial Sequence
  source: /note=“Description of Artificial Sequence Synthetic peptide”
ARSLRRRSTEHAGFDV

SEQ ID NO: 233 - protein, 123 aa, Artificial Sequence
  source: /note=“Description of Artificial Sequence Synthetic polypeptide”
EVQLVESGGGLVQPGGSLRLSCAASGFTFSSYWISWVRQAPGKGLEWVANIKQSGSETYYVESVKGRFTISRDNAKNSLYLQMNSLRAEDTAVYYCARSLRRRSTEHAGFDVWGQGTLVTVSS

SEQ ID NO: 234 - DNA, 369 bp, Artificial Sequence
  source: /note=“Description of Artificial Sequence Synthetic polynucleotide”
gaagtgcagctggtggaaagcggcggtggcctggtgcagccaggtggtagcctgcgcctgagctgcgccgccagcggctttacctttagcagctattggattagctgggttcgccaggccccaggcaaaggcctggaatgggtggcgaacatcaaacagagcggcagcgagacctactatgtggagagcgtgaaaggccgctttaccattagccgcgataacgccaaaaacagcctgtatctgcaaatgaacagcctgcgggccgaagataccgccgtgtattattgcgcgcgtagcctgcgtcgtcgtagcactgagcacgcaggattcgacgtttggggccagggcaccctggttactgtctcgagc

SEQ ID NO: 235 - protein, 453 aa, Artificial Sequence
  source: /note=“Description of Artificial Sequence Synthetic polypeptide”
EVQLVESGGGLVQPGGSLRLSCAASGFTFSSYWISWVRQAPGKGLEWVANIKQSGSETYYVESVKGRFTISRDNAKNSLYLQMNSLRAEDTAVYYCARSLRRRSTEHAGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - DNA, 1359 bp, Artificial Sequence
  source: /note=“Description of Artificial Sequence Synthetic polynucleotide”
gaagtgcagctggtggaaagcggcggtggcctggtgcagccaggtggtagcctgcgcctgagctgcgccgccagcggctttacctttagcagctattggattagctgggttcgccaggccccaggcaaaggcctggaatgggtggcgaacatcaaacagagcggcagcgagacctactatgtggagagcgtgaaaggccgctttaccattagccgcgataacgccaaaaacagcctgtatctgcaaatgaacagcctgcgggccgaagataccgccgtgtattattgcgcgcgtagcctgcgtcgtcgtagcactgagcacgcaggattcgacgtttggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 237 - protein, 11 aa, Artificial Sequence
  source: /note=“Description of Artificial Sequence Synthetic peptide”
RASQGISNYLA

SEQ ID NO: 238 - protein, 7 aa, Artificial Sequence
  source: /note=“Description of Artificial Sequence Synthetic peptide”
AASTLQS

SEQ ID NO: 239 - protein, 9 aa, Artificial Sequence
  source: /note=“Description of Artificial Sequence Synthetic peptide”
QQADKFPYT

SEQ ID NO: 240 - protein, 7 aa, Artificial Sequence
  source: /note=“Description of Artificial Sequence Synthetic peptide”
SQGISNY

SEQ ID NO: 241 - protein, 3 aa, Artificial Sequence
  source: /note=“Description of Artificial Sequence Synthetic peptide”
AAS

SEQ ID NO: 242 - protein, 6 aa, Artificial Sequence
  source: /note=“Description of Artificial Sequence Synthetic peptide”
ADKFPY

SEQ ID NO: 243 - protein, 6 aa, Artificial Sequence
  source: /note=“Description of Artificial Sequence Synthetic peptide”
QGISNY

SEQ ID NO: 244 - protein, 107 aa, Artificial Sequence
  source: /note=“Description of Artificial Sequence Synthetic polypeptide”
DIQMTQSPSSLSASVGDRVTITCRASQGISNYLAWYQQKPGKVPKLLIYAASTLQSGVPSRFSGSGSGTDFTLTISSLQPEDVATYYCQQADKFPYTFGQGTKVEIK

SEQ ID NO: 245 - DNA, 321 bp, Artificial Sequence
  source: /note=“Description of Artificial Sequence Synthetic polynucleotide”
gatattcagatgacccagagcccgagcagcctgagcgcaagcgtgggcgatcgcgtgaccattacctgccgcgccagccagggcattagcaactatctggcctggtatcagcagaaaccgggcaaagtgccgaaactgctgatctatgccgccagcaccctgcaaagcggcgtgccaagtcgctttagcggcagcggtagcggcaccgatttcaccctgaccattagcagcctgcaaccggaagacgtggcgacctattattgccagcaggctgacaaattcccgtacaccttcggccagggtaccaaagtggaaatcaag

SEQ ID NO: 246 - protein, 214 aa, Artificial Sequence
  source: /note=“Description of Artificial Sequence Synthetic polypeptide”
DIQMTQSPSSLSASVGDRVTITCRASQGISNYLAWYQQKPGKVPKLLIYAASTLQSGVPSRFSGSGSGTDFTLTISSLQPEDVATYYCQQADKFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - DNA, 642 bp, Artificial Sequence
  source: /note=“Description of Artificial Sequence Synthetic polynucleotide”
gatattcagatgacccagagcccgagcagcctgagcgcaagcgtgggcgatcgcgtgaccattacctgccgcgccagccagggcattagcaactatctggcctggtatcagcagaaaccgggcaaagtgccgaaactgctgatctatgccgccagcaccctgcaaagcggcgtgccaagtcgctttagcggcagcggtagcggcaccgatttcaccctgaccattagcagcctgcaaccggaagacgtggcgacctattattgccagcaggctgacaaattcccgtacaccttcggccagggtaccaaagtggaaatcaagcggaccgtggccgctccctccgtgttcatcttcccacccagcgacgagcagctgaagtccggcacagccagcgtcgtgtgcctgctgaacaacttctacccccgcgaggccaaagtgcagtggaaggtggacaacgccctccagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgt

SEQ ID NO: 248 - protein, 10 aa, Artificial Sequence
  source: /note=“Description of Artificial Sequence Synthetic peptide”
GFTFSSYSMN

SEQ ID NO: 249 - protein, 17 aa, Artificial Sequence
  source: /note=“Description of Artificial Sequence Synthetic peptide”
SISSSSSYIYYADSVKG

SEQ ID NO: 250 - protein, 11 aa, Artificial Sequence
  source: /note=“Description of Artificial Sequence Synthetic peptide”
SGYRGVYGFDY

SEQ ID NO: 251 - protein, 5 aa, Artificial Sequence
  source: /note=“Description of Artificial Sequence Synthetic peptide”
SYSMN

SEQ ID NO: 252 - protein, 6 aa, Artificial Sequence
  source: /note=“Description of Artificial Sequence Synthetic peptide”
SSSSSY

SEQ ID NO: 253 - protein, 8 aa, Artificial Sequence
  source: /note=“Description of Artificial Sequence Synthetic peptide”
GFTFSSYS

SEQ ID NO: 254 - protein, 8 aa, Artificial Sequence
  source: /note=“Description of Artificial Sequence Synthetic peptide”
ISSSSSYI

SEQ ID NO: 255 - protein, 13 aa, Artificial Sequence
  source: /note=“Description of Artificial Sequence Synthetic peptide”
ARSGYRGVYGFDY

SEQ ID NO: 256 - protein, 120 aa, Artificial Sequence
  source: /note=“Description of Artificial Sequence Synthetic polypeptide”
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSGYRGVYGFDYWGQGTLVTVSS

SEQ ID NO: 257 - DNA, 360 bp, Artificial Sequence
  source: /note=“Description of Artificial Sequence Synthetic polynucleotide”
gaagtgcagctggtggaaagcggcggtggcctggtgaaaccaggcggtagcctgcgcctgagctgcgccgccagcggctttacctttagcagctatagcatgaactgggttcgccaggccccaggcaaaggcctggaatgggttagcagcatcagcagcagtagcagctatatctattacgccgatagcgtgaaaggccgctttaccattagccgcgataacgccaaaaacagcctgtatctgcaaatgaacagcctgcgggccgaagataccgccgtgtattattgcgcgcgaagcggatatcgtggagtttacggatttgattattggggccagggcaccctggttactgtctcgagc

SEQ ID NO: 258 - protein, 450 aa, Artificial Sequence
  source: /note=“Description of Artificial Sequence Synthetic polypeptide”
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSGYRGVYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - DNA, 1350 bp, Artificial Sequence
  source: /note=“Description of Artificial Sequence Synthetic polynucleotide”
gaagtgcagctggtggaaagcggcggtggcctggtgaaaccaggcggtagcctgcgcctgagctgcgccgccagcggctttacctttagcagctatagcatgaactgggttcgccaggccccaggcaaaggcctggaatgggttagcagcatcagcagcagtagcagctatatctattacgccgatagcgtgaaaggccgctttaccattagccgcgataacgccaaaaacagcctgtatctgcaaatgaacagcctgcgggccgaagataccgccgtgtattattgcgcgcgaagcggatatcgtggagtttacggatttgattattggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 260 - protein, 11 aa, Artificial Sequence
  source: /note=“Description of Artificial Sequence Synthetic peptide”
RASQGISSWLA

SEQ ID NO: 261 - protein, 7 aa, Artificial Sequence
  source: /note=“Description of Artificial Sequence Synthetic peptide”
AASSLQS

SEQ ID NO: 262 - protein, 9 aa, Artificial Sequence
  source: /note=“Description of Artificial Sequence Synthetic peptide”
QQYYHSPLT

SEQ ID NO: 263 - protein, 7 aa, Artificial Sequence
  source: /note=“Description of Artificial Sequence Synthetic peptide”
SQGISSW

SEQ ID NO: 264 - protein, 6 aa, Artificial Sequence
  source: /note=“Description of Artificial Sequence Synthetic peptide”
YYHSPL

SEQ ID NO: 265 - protein, 6 aa, Artificial Sequence
  source: /note=“Description of Artificial Sequence Synthetic peptide”
QGISSW

SEQ ID NO: 266 - protein, 107 aa, Artificial Sequence
  source: /note=“Description of Artificial Sequence Synthetic polypeptide”
DIQMTQSPSSVSASVGDRVTITCRASQGISSWLAWYQQKPGKAPKLLIYAASSLQSGVPSRFSGSGSGTDFTLTISSLQPEDFATYYCQQYYHSPLTFGQGTKVEIK

SEQ ID NO: 267 - DNA, 321 bp, Artificial Sequence
  source: /note=“Description of Artificial Sequence Synthetic polynucleotide”
gatattcagatgacccagagcccgagcagcgttagcgccagcgtgggcgatcgcgtgaccattacctgccgcgccagtcagggcattagcagctggctggcctggtatcagcagaaaccgggcaaagccccgaaactgctgatctatgccgccagcagcctgcaaagcggcgtgccaagtcgctttagcggcagcggtagcggcaccgatttcaccctgaccattagcagtctgcaaccggaagactttgccacctattattgccagcagtactaccattctccgctgaccttcggccagggtaccaaagtggaaatcaag

SEQ ID NO: 268 - protein, 214 aa, Artificial Sequence
  source: /note=“Description of Artificial Sequence Synthetic polypeptide”
DIQMTQSPSSVSASVGDRVTITCRASQGISSWLAWYQQKPGKAPKLLIYAASSLQSGVPSRFSGSGSGTDFTLTISSLQPEDFATYYCQQYYHSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - DNA, 642 bp, Artificial Sequence
  source: /note=“Description of Artificial Sequence Synthetic polynucleotide”
gatattcagatgacccagagcccgagcagcgttagcgccagcgtgggcgatcgcgtgaccattacctgccgcgccagtcagggcattagcagctggctggcctggtatcagcagaaaccgggcaaagccccgaaactgctgatctatgccgccagcagcctgcaaagcggcgtgccaagtcgctttagcggcagcggtagcggcaccgatttcaccctgaccattagcagtctgcaaccggaagactttgccacctattattgccagcagtactaccattctccgctgaccttcggccagggtaccaaagtggaaatcaagcggaccgtggccgctccctccgtgttcatcttcccacccagcgacgagcagctgaagtccggcacagccagcgtcgtgtgcctgctgaacaacttctacccccgcgaggccaaagtgcagtggaaggtggacaacgccctccagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgt

SEQ ID NO: 270 - protein, 10 aa, Artificial Sequence
  source: /note=“Description of Artificial Sequence Synthetic peptide”
GFTFSSYAIS

SEQ ID NO: 271 - protein, 17 aa, Artificial Sequence
  source: /note=“Description of Artificial Sequence Synthetic peptide”
AISGSGGSTYYAESVKG

SEQ ID NO: 272 - protein, 12 aa, Artificial Sequence
  source: /note=“Description of Artificial Sequence Synthetic peptide”
ESGYVYYLKFDY

SEQ ID NO: 273 - protein, 5 aa, Artificial Sequence
  source: /note=“Description of Artificial Sequence Synthetic peptide”
SYAIS

SEQ ID NO: 274 - protein, 6 aa, Artificial Sequence
  source: /note=“Description of Artificial Sequence Synthetic peptide”
SGSGGS

SEQ ID NO: 275 - protein, 8 aa, Artificial Sequence
  source: /note=“Description of Artificial Sequence Synthetic peptide”
GFTFSSYA

SEQ ID NO: 276 - protein, 8 aa, Artificial Sequence
  source: /note=“Description of Artificial Sequence Synthetic peptide”
ISGSGGST

SEQ ID NO: 277 - protein, 14 aa, Artificial Sequence
  source: /note=“Description of Artificial Sequence Synthetic peptide”
ARESGYVYYLKFDY

SEQ ID NO: 278 - protein, 121 aa, Artificial Sequence
  source: /note=“Description of Artificial Sequence Synthetic polypeptide”
EVQLLESGGGLVQPGGSLRLSCAASGFTFSSYAISWVRQAPGKGLEWVSAISGSGGSTYYAESVKGRFTISRDNSKNTLYLQMNSLRAEDTAVYYCARESGYVYYLKFDYWGQGTLVTVSS

SEQ ID NO: 279 - DNA, 363 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcggctttacctttagcagctatgccattagctgggtgcgccaagcaccaggcaaaggcctggaatgggtgagcgccattagcggcagcggtggcagcacctattatgccgagagcgtgaaaggtcgctttaccattagtcgcgataacagcaaaaacaccctgtatctgcaaatgaacagcctgcgggcagaagataccgcagtttattattgcgcgcgtgagagcggatacgtttactatctgaaattcgattattggggccagggcaccctggttactgtctcgagc

SEQ ID NO: 280 - protein, 451 aa, Artificial Sequence
  source: /note=“Description of Artificial Sequence Synthetic polypeptide”
EVQLLESGGGLVQPGGSLRLSCAASGFTFSSYAISWVRQAPGKGLEWVSAISGSGGSTYYAESVKGRFTISRDNSKNTLYLQMNSLRAEDTAVYYCARESGYVYYLKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - DNA, 1353 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcggctttacctttagcagctatgccattagctgggtgcgccaagcaccaggcaaaggcctggaatgggtgagcgccattagcggcagcggtggcagcacctattatgccgagagcgtgaaaggtcgctttaccattagtcgcgataacagcaaaaacaccctgtatctgcaaatgaacagcctgcgggcagaagataccgcagtttattattgcgcgcgtgagagcggatacgtttactatctgaaattcgattattggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 282 - protein, 11 aa, Artificial Sequence
  source: /note=“Description of Artificial Sequence Synthetic peptide”
RASQSISSYLN

SEQ ID NO: 283 - protein, 9 aa, Artificial Sequence
  source: /note=“Description of Artificial Sequence Synthetic peptide”
QQHVRVPIT

SEQ ID NO: 284 - protein, 7 aa, Artificial Sequence
  source: /note=“Description of Artificial Sequence Synthetic peptide”
SQSISSY

SEQ ID NO: 285 - protein, 6 aa, Artificial Sequence
  source: /note=“Description of Artificial Sequence Synthetic peptide”
HVRVPI

SEQ ID NO: 286 - protein, 6 aa, Artificial Sequence
  source: /note=“Description of Artificial Sequence Synthetic peptide”
QSISSY

SEQ ID NO: 287 - protein, 107 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQHVRVPITFGQGTKVEIK

SEQ ID NO: 288 - DNA, 321 bp, Artificial Sequence
  source: /note=“Description of Artificial Sequence Synthetic polynucleotide”
gatattcagatgacccagagcccgagcagcctgagcgccagcgtgggtgatcgcgtgaccattacctgtcgcgcaagccagagcattagcagctatctgaactggtatcagcagaaaccaggcaaagccccaaaactgctgatttatgccgcaagcagcctgcaaagcggtgtgccgagccgctttagcggcagcggtagcggcaccgattttaccctgaccattagtagcctgcaaccggaagactttgccacctattattgccagcagcatgttcgtgttccgatcaccttcggccagggtaccaaagtggaaatcaag

SEQ ID NO: 289 - protein, 214 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQHVRVPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 290 - DNA, 642 bp, Artificial Sequence
  source: /note=“Description of Artificial Sequence Synthetic polynucleotide”
gatattcagatgacccagagcccgagcagcctgagcgccagcgtgggtgatcgcgtgaccattacctgtcgcgcaagccagagcattagcagctatctgaactggtatcagcagaaaccaggcaaagccccaaaactgctgatttatgccgcaagcagcctgcaaagcggtgtgccgagccgctttagcggcagcggtagcggcaccgattttaccctgaccattagtagcctgcaaccggaagactttgccacctattattgccagcagcatgttcgtgttccgatcaccttcggccagggtaccaaagtggaaatcaagcggaccgtggccgctccctccgtgttcatcttcccacccagcgacgagcagctgaagtccggcacagccagcgtcgtgtgcctgctgaacaacttctacccccgcgaggccaaagtgcagtggaaggtggacaacgccctccagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgt

SEQ ID NO: 291 - protein, 10 aa, Artificial Sequence
  source: /note=“Description of Artificial Sequence Synthetic peptide”
GFTFSNYWIS

SEQ ID NO: 292 - protein, 19 aa, Artificial Sequence
  source: /note=“Description of Artificial Sequence Synthetic peptide”
RIKSKTYGGTTDYAEPVKG

SEQ ID NO: 293 - protein, 17 aa, Artificial Sequence
  source: /note=“Description of Artificial Sequence Synthetic peptide”
EKYSIRARGHGDYGFDV

SEQ ID NO: 294 - protein, 5 aa, Artificial Sequence
  source: /note=“Description of Artificial Sequence Synthetic peptide”
NYWIS

SEQ ID NO: 295 - protein, 7 aa, Artificial Sequence
  source: /note=“Description of Artificial Sequence Synthetic peptide”
GFTFSNY

SEQ ID NO: 296 - protein, 8 aa, Artificial Sequence
  source: /note=“Description of Artificial Sequence Synthetic peptide”
KSKTYGGT

SEQ ID NO: 297 - protein, 8 aa, Artificial Sequence
  source: /note=“Description of Artificial Sequence Synthetic peptide”
GFTFSNYW

SEQ ID NO: 298 - protein, 10 aa, Artificial Sequence
  source: /note=“Description of Artificial Sequence Synthetic peptide”
IKSKTYGGTT

SEQ ID NO: 299 - protein, 19 aa, Artificial Sequence
  source: /note=“Description of Artificial Sequence Synthetic peptide”
AREKYSIRARGHGDYGFDV

SEQ ID NO: 300 - protein, 128 aa, Artificial Sequence
  source: /note=“Description of Artificial Sequence Synthetic polypeptide”
EVQLVESGGGLVKPGGSLRLSCAASGFTFSNYWISWVRQAPGKGLEWVGRIKSKTYGGTTDYAEPVKGRFTISRDDSKNTLYLQMNSLKTEDTAVYYCAREKYSIRARGHGDYGFDVWGQGTLVTVSS

SEQ ID NO: 301 - DNA, 384 bp, Artificial Sequence
  source: /note=“Description of Artificial Sequence Synthetic polynucleotide”
gaagtgcagctggtggaaagcggcggtggcctggtgaaaccaggcggtagcctgcgcctgagctgcgccgccagcggctttacctttagcaactattggattagctgggttcgccaggccccaggcaaaggcctggaatgggttggccgcatcaaaagcaaaacctatggcggcaccaccgattatgccgagccagtgaaaggccgctttaccattagccgcgacgatagcaaaaacaccctgtacctgcaaatgaacagcctgaaaaccgaagataccgccgtgtattattgcgcgcgtgagaaatattccatccgtgcacgtggtcacggagactacggatttgatgtgtggggccagggcaccctggttactgtctcgagc

SEQ ID NO: 302 - protein, 458 aa, Artificial Sequence
  source: /note=“Description of Artificial Sequence Synthetic polypeptide”
EVQLVESGGGLVKPGGSLRLSCAASGFTFSNYWISWVRQAPGKGLEWVGRIKSKTYGGTTDYAEPVKGRFTISRDDSKNTLYLQMNSLKTEDTAVYYCAREKYSIRARGHGDYGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - DNA, 1374 bp, Artificial Sequence
  source: /note=“Description of Artificial Sequence Synthetic polynucleotide”
gaagtgcagctggtggaaagcggcggtggcctggtgaaaccaggcggtagcctgcgcctgagctgcgccgccagcggctttacctttagcaactattggattagctgggttcgccaggccccaggcaaaggcctggaatgggttggccgcatcaaaagcaaaacctatggcggcaccaccgattatgccgagccagtgaaaggccgctttaccattagccgcgacgatagcaaaaacaccctgtacctgcaaatgaacagcctgaaaaccgaagataccgccgtgtattattgcgcgcgtgagaaatattccatccgtgcacgtggtcacggagactacggatttgatgtgtggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 304 - protein, 9 aa, Artificial Sequence
  source: /note=“Description of Artificial Sequence Synthetic peptide”
QQGYHAPFT

SEQ ID NO: 305 - protein, 6 aa, Artificial Sequence
  source: /note=“Description of Artificial Sequence Synthetic peptide”
GYHAPF

SEQ ID NO: 306 - protein, 107 aa, Artificial Sequence
  source: /note=“Description of Artificial Sequence Synthetic polypeptide”
DIQMTQSPSSLSASVGDRVTITCRASQGISNYLAWYQQKPGKVPKLLIYAASTLQSGVPSRFSGSGSGTDFTLTISSLQPEDVATYYCQQGYHAPFTFGQGTKVEIK

SEQ ID NO: 307 - DNA, 321 bp, Artificial Sequence
  source: /note=“Description of Artificial Sequence Synthetic polynucleotide”
gatattcagatgacccagagcccgagcagcctgagcgcaagcgtgggcgatcgcgtgaccattacctgccgcgccagccagggcattagcaactatctggcctggtatcagcagaaaccgggcaaagtgccgaaactgctgatctatgccgccagcaccctgcaaagcggcgtgccaagtcgctttagcggcagcggtagcggcaccgatttcaccctgaccattagcagcctgcaaccggaagacgtggcgacctattattgccagcagggttaccatgctccgttcaccttcggccagggtaccaaagtggaaatcaag

SEQ ID NO: 308 - protein, 214 aa, Artificial Sequence
  source: /note=“Description of Artificial Sequence Synthetic polypeptide”
DIQMTQSPSSLSASVGDRVTITCRASQGISNYLAWYQQKPGKVPKLLIYAASTLQSGVPSRFSGSGSGTDFTLTISSLQPEDVATYYCQQGYHAPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 309 - DNA, 642 bp, Artificial Sequence
  source: /note=“Description of Artificial Sequence Synthetic polynucleotide”
gatattcagatgacccagagcccgagcagcctgagcgcaagcgtgggcgatcgcgtgaccattacctgccgcgccagccagggcattagcaactatctggcctggtatcagcagaaaccgggcaaagtgccgaaactgctgatctatgccgccagcaccctgcaaagcggcgtgccaagtcgctttagcggcagcggtagcggcaccgatttcaccctgaccattagcagcctgcaaccggaagacgtggcgacctattattgccagcagggttaccatgctccgttcaccttcggccagggtaccaaagtggaaatcaagcggaccgtggccgctccctccgtgttcatcttcccacccagcgacgagcagctgaagtccggcacagccagcgtcgtgtgcctgctgaacaacttctacccccgcgaggccaaagtgcagtggaaggtggacaacgccctccagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgt

SEQ ID NO: 310 - protein, 10 aa, Artificial Sequence
  source: /note=“Description of Artificial Sequence Synthetic peptide”
GYSFTSYWIS

SEQ ID NO: 311 - protein, 17 aa, Artificial Sequence
  source: /note=“Description of Artificial Sequence Synthetic peptide”
IIYPGTSYTRYSPSFQG

SEQ ID NO: 312 - protein, 11 aa, Artificial Sequence
  source: /note=“Description of Artificial Sequence Synthetic peptide”
GAVAGQLGFDH

SEQ ID NO: 313 - protein, 6 aa, Artificial Sequence
  source: /note=“Description of Artificial Sequence Synthetic peptide”
YPGTSY

SEQ ID NO: 314 - protein, 8 aa, Artificial Sequence
  source: /note=“Description of Artificial Sequence Synthetic peptide”
IYPGTSYT

SEQ ID NO: 315 - protein, 13 aa, Artificial Sequence
  source: /note=“Description of Artificial Sequence Synthetic peptide”
ARGAVAGQLGFDH

SEQ ID NO: 316 - protein, 120 aa, Artificial Sequence
  source: /note=“Description of Artificial Sequence Synthetic polypeptide”
EVQLVQSGAEVKKPGESLKISCKGSGYSFTSYWISWVRQMPGKGLEWMGIIYPGTSYTRYSPSFQGQVTISADKSISTAYLQWSSLKASDTAMYYCARGAVAGQLGFDHWGQGTLVTVSS

SEQ ID NO: 317 - DNA, 360 bp, Artificial Sequence
  source: /note=“Description of Artificial Sequence Synthetic polynucleotide”
gaagtgcagctggtgcagagcggtgccgaagtgaaaaaaccgggcgaaagcctgaaaatcagctgcaaaggcagcggctatagctttaccagctattggattagctgggttcgccagatgccgggcaaaggcctggaatggatgggcattatctatccgggcaccagctatacccgctatagcccgagctttcagggccaggttacaattagcgccgacaaaagcatcagcaccgcctatctgcaatggagcagcctgaaagccagcgataccgccatgtattattgcgcgcgtggtgcagttgcaggacaactgggatttgatcactggggccagggcaccctggttactgtctcgagc

SEQ ID NO: 318 - protein, 450 aa, Artificial Sequence
  source: /note=“Description of Artificial Sequence Synthetic polypeptide”
EVQLVQSGAEVKKPGESLKISCKGSGYSFTSYWISWVRQMPGKGLEWMGIIYPGTSYTRYSPSFQGQVTISADKSISTAYLQWSSLKASDTAMYYCARGAVAGQLGFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - DNA, 1350 bp, Artificial Sequence
  source: /note=“Description of Artificial Sequence Synthetic polynucleotide”
gaagtgcagctggtgcagagcggtgccgaagtgaaaaaaccgggcgaaagcctgaaaatcagctgcaaaggcagcggctatagctttaccagctattggattagctgggttcgccagatgccgggcaaaggcctggaatggatgggcattatctatccgggcaccagctatacccgctatagcccgagctttcagggccaggttacaattagcgccgacaaaagcatcagcaccgcctatctgcaatggagcagcctgaaagccagcgataccgccatgtattattgcgcgcgtggtgcagttgcaggacaactgggatttgatcactggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 320 - protein, 14 aa, Artificial Sequence
  source: /note=“Description of Artificial Sequence Synthetic peptide”
TGSSSNIGAGYDVH

SEQ ID NO: 321 - protein, 7 aa, Artificial Sequence
  source: /note=“Description of Artificial Sequence Synthetic peptide”
GNSNRPS

SEQ ID NO: 322 - protein, 11 aa, Artificial Sequence
  source: /note=“Description of Artificial Sequence Synthetic peptide”
QSYYTSSHGPV

SEQ ID NO: 323 - protein, 10 aa, Artificial Sequence
  source: /note=“Description of Artificial Sequence Synthetic peptide”
SSSNIGAGYD

SEQ ID NO: 324 - protein, 3 aa, Artificial Sequence
  source: /note=“Description of Artificial Sequence Synthetic peptide”
GNS

SEQ ID NO: 325 - protein, 8 aa, Artificial Sequence
  source: /note=“Description of Artificial Sequence Synthetic peptide”
YYTSSHGP

SEQ ID NO: 326 - protein, 9 aa, Artificial Sequence
  source: /note=“Description of Artificial Sequence Synthetic peptide”
SSNIGAGYD

SEQ ID NO: 327 - protein, 111 aa, Artificial Sequence
  source: /note=“Description of Artificial Sequence Synthetic polypeptide”
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYYTSSHGPVFGGGTKLTVL

SEQ ID NO: 328 - DNA, 333 bp, Artificial Sequence
  source: /note=“Description of Artificial Sequence Synthetic polynucleotide”
cagagcgtgctgacccagccaccaagcgtgagcggtgcaccaggtcagcgcgtgaccattagctgcaccggcagcagcagcaacattggcgcaggctatgatgtgcattggtatcagcagctgccaggcaccgcaccgaaactgctgatttatggcaacagcaatcgcccaagcggtgtgccggatcgctttagcggcagcaaaagcggcaccagcgccagcctggcgattaccggtctgcaagccgaagacgaagccgattattactgccagtcttactacacttcttctcatggtccggtgtttggcggcggtaccaagctgaccgtgctg

SEQ ID NO: 329 - protein, 217 aa, Artificial Sequence
  source: /note=“Description of Artificial Sequence Synthetic polypeptide”
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYYTSSHGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 330 - DNA, 651 bp, Artificial Sequence
  source: /note=“Description of Artificial Sequence Synthetic polynucleotide”
cagagcgtgctgacccagccaccaagcgtgagcggtgcaccaggtcagcgcgtgaccattagctgcaccggcagcagcagcaacattggcgcaggctatgatgtgcattggtatcagcagctgccaggcaccgcaccgaaactgctgatttatggcaacagcaatcgcccaagcggtgtgccggatcgctttagcggcagcaaaagcggcaccagcgccagcctggcgattaccggtctgcaagccgaagacgaagccgattattactgccagtcttactacacttcttctcatggtccggtgtttggcggcggtaccaagctgaccgtgctgggccagcccaaagccgcccctagcgtgaccctgttccccccaagcagcgaggaactccaggccaacaaggccaccctcgtgtgcctgatcagcgacttctaccctggcgccgtgaccgtggcctggaaggccgatagcagccctgtgaaggccggcgtggaaaccaccacccccagcaagcagagcaacaacaaatacgccgccagcagctacctgagcctgacccccgagcagtggaagtcccacagatcctacagctgccaggtcacacacgagggcagcaccgtggaaaagaccgtggcccccaccgagtgcagc

SEQ ID NO: 331 - protein, 13 aa, Artificial Sequence
  source: /note=“Description of Artificial Sequence Synthetic peptide”
PYLGDRRSYGFDH

SEQ ID NO: 332 - protein, 15 aa, Artificial Sequence
  source: /note=“Description of Artificial Sequence Synthetic peptide”
ARPYLGDRRSYGFDH

SEQ ID NO: 333 - protein, 122 aa, Artificial Sequence
  source: /note=“Description of Artificial Sequence Synthetic polypeptide”
EVQLLESGGGLVQPGGSLRLSCAASGFTFSSYAISWVRQAPGKGLEWVSAISGSGGSTYYAESVKGRFTISRDNSKNTLYLQMNSLRAEDTAVYYCARPYLGDRRSYGFDHWGQGTLVTVSS

SEQ ID NO: 334 - DNA, 366 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcggctttacctttagcagctatgccattagctgggtgcgccaagcaccaggcaaaggcctggaatgggtgagcgccattagcggcagcggtggcagcacctattatgccgagagcgtgaaaggtcgctttaccattagtcgcgataacagcaaaaacaccctgtatctgcaaatgaacagcctgcgggcagaagataccgcagtttattattgcgcgcgaccttatctgggtgaccgtcgtagctatggtttcgaccactggggccagggcaccctggttactgtctcgagc

SEQ ID NO: 335 - protein, 452 aa, Artificial Sequence
  source: /note=“Description of Artificial Sequence Synthetic polypeptide”
EVQLLESGGGLVQPGGSLRLSCAASGFTFSSYAISWVRQAPGKGLEWVSAISGSGGSTYYAESVKGRFTISRDNSKNTLYLQMNSLRAEDTAVYYCARPYLGDRRSYGFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - DNA, 1356 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcggctttacctttagcagctatgccattagctgggtgcgccaagcaccaggcaaaggcctggaatgggtgagcgccattagcggcagcggtggcagcacctattatgccgagagcgtgaaaggtcgctttaccattagtcgcgataacagcaaaaacaccctgtatctgcaaatgaacagcctgcgggcagaagataccgcagtttattattgcgcgcgaccttatctgggtgaccgtcgtagctatggtttcgaccactggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 337 - protein, 14 aa, Artificial Sequence
  source: /note=“Description of Artificial Sequence Synthetic peptide”
TGTSSDVGSYNLVS

SEQ ID NO: 338 - protein, 7 aa, Artificial Sequence
  source: /note=“Description of Artificial Sequence Synthetic peptide”
EGSKRPS

SEQ ID NO: 339 - protein, 11 aa, Artificial Sequence
  source: /note=“Description of Artificial Sequence Synthetic peptide”
SSYGFHIVVVV

SEQ ID NO: 340 - protein, 10 aa, Artificial Sequence
  source: /note=“Description of Artificial Sequence Synthetic peptide”
TSSDVGSYNL

SEQ ID NO: 341 - protein, 3 aa, Artificial Sequence
  source: /note=“Description of Artificial Sequence Synthetic peptide”
EGS

SEQ ID NO: 342 - protein, 8 aa, Artificial Sequence
  source: /note=“Description of Artificial Sequence Synthetic peptide”
YGFHIVVV

SEQ ID NO: 343 - protein, 9 aa, Artificial Sequence
  source: /note=“Description of Artificial Sequence Synthetic peptide”
SSDVGSYNL

SEQ ID NO: 344 - protein, 111 aa, Artificial Sequence
  source: /note=“Description of Artificial Sequence Synthetic polypeptide”
QSALTQPASVSGSPGQSITISCTGTSSDVGSYNLVSWYQQHPGKAPKLMIYEGSKRPSGVSNRFSGSKSGNTASLTISGLQAEDEADYYCSSYGFHIVVVVFGGGTKLTVL

SEQ ID NO: 345 - DNA, 333 bp, Artificial Sequence
  source: /note=“Description of Artificial Sequence Synthetic polynucleotide”
cagagcgccctgacccagccagccagcgttagcggtagcccaggccagagcattaccattagctgcaccggcaccagcagcgacgtgggcagctataacctggttagctggtatcagcagcatccgggcaaagccccgaaactgatgatctatgaaggcagcaaacgcccgagcggcgttagcaaccgctttagtggcagcaaaagcggcaacaccgccagcctgaccattagcggcctgcaagccgaagacgaagccgattattactgctcctcttacggtttccatatcgttgttgttgtgtttggcggcggtaccaagctgaccgtgctg

SEQ ID NO: 346 - protein, 217 aa, Artificial Sequence
  source: /note=“Description of Artificial Sequence Synthetic polypeptide”
QSALTQPASVSGSPGQSITISCTGTSSDVGSYNLVSWYQQHPGKAPKLMIYEGSKRPSGVSNRFSGSKSGNTASLTISGLQAEDEADYYCSSYGFHIVVVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 347 - DNA, 651 bp, Artificial Sequence
  source: /note=“Description of Artificial Sequence Synthetic polynucleotide”
cagagcgccctgacccagccagccagcgttagcggtagcccaggccagagcattaccattagctgcaccggcaccagcagcgacgtgggcagctataacctggttagctggtatcagcagcatccgggcaaagccccgaaactgatgatctatgaaggcagcaaacgcccgagcggcgttagcaaccgctttagtggcagcaaaagcggcaacaccgccagcctgaccattagcggcctgcaagccgaagacgaagccgattattactgctcctcttacggtttccatatcgttgttgttgtgtttggcggcggtaccaagctgaccgtgctgggccagcccaaagccgcccctagcgtgaccctgttccccccaagcagcgaggaactccaggccaacaaggccaccctcgtgtgcctgatcagcgacttctaccctggcgccgtgaccgtggcctggaaggccgatagcagccctgtgaaggccggcgtggaaaccaccacccccagcaagcagagcaacaacaaatacgccgccagcagctacctgagcctgacccccgagcagtggaagtcccacagatcctacagctgccaggtcacacacgagggcagcaccgtggaaaagaccgtggcccccaccgagtgcagc

SEQ ID NO: 348 - protein, 11 aa, Artificial Sequence
  source: /note=“Description of Artificial Sequence Synthetic peptide”
GSLPGLLGFDH

SEQ ID NO: 349 - protein, 13 aa, Artificial Sequence
  source: /note=“Description of Artificial Sequence Synthetic peptide”
ARGSLPGLLGFDH

SEQ ID NO: 350 - protein, 120 aa, Artificial Sequence
  source: /note=“Description of Artificial Sequence Synthetic polypeptide”
EVQLVQSGAEVKKPGESLKISCKGSGYSFTSYWISWVRQMPGKGLEWMGIIYPGTSYTRYSPSFQGQVTISADKSISTAYLQWSSLKASDTAMYYCARGSLPGLLGFDHWGQGTLVTVSS

SEQ ID NO: 351 - DNA, 360 bp, Artificial Sequence
  source: /note=“Description of Artificial Sequence Synthetic polynucleotide”
gaagtgcagctggtgcagagcggtgccgaagtgaaaaaaccgggcgaaagcctgaaaatcagctgcaaaggcagcggctatagctttaccagctattggattagctgggttcgccagatgccgggcaaaggcctggaatggatgggcattatctatccgggcaccagctatacccgctatagcccgagctttcagggccaggttacaattagcgccgacaaaagcatcagcaccgcctatctgcaatggagcagcctgaaagccagcgataccgccatgtattattgcgcgcgtggaagcctgcctggtctgctgggttttgatcactggggccagggcaccctggttactgtctcgagc

SEQ ID NO: 352 - protein, 450 aa, Artificial Sequence
  source: /note=“Description of Artificial Sequence Synthetic polypeptide”
EVQLVQSGAEVKKPGESLKISCKGSGYSFTSYWISWVRQMPGKGLEWMGIIYPGTSYTRYSPSFQGQVTISADKSISTAYLQWSSLKASDTAMYYCARGSLPGLLGFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - DNA, 1350 bp, Artificial Sequence
  source: /note=“Description of Artificial Sequence Synthetic polynucleotide”
gaagtgcagctggtgcagagcggtgccgaagtgaaaaaaccgggcgaaagcctgaaaatcagctgcaaaggcagcggctatagctttaccagctattggattagctgggttcgccagatgccgggcaaaggcctggaatggatgggcattatctatccgggcaccagctatacccgctatagcccgagctttcagggccaggttacaattagcgccgacaaaagcatcagcaccgcctatctgcaatggagcagcctgaaagccagcgataccgccatgtattattgcgcgcgtggaagcctgcctggtctgctgggttttgatcactggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 354 - protein, 7 aa, Artificial Sequence
  source: /note=“Description of Artificial Sequence Synthetic peptide”
GNSNRPN

SEQ ID NO: 355 - protein, 11 aa, Artificial Sequence
  source: /note=“Description of Artificial Sequence Synthetic peptide”
QSYDSPTSSSV

SEQ ID NO: 356 - protein, 8 aa, Artificial Sequence
  source: /note=“Description of Artificial Sequence Synthetic peptide”
YDSPTSSS

SEQ ID NO: 357 - protein, 111 aa, Artificial Sequence
  source: /note=“Description of Artificial Sequence Synthetic polypeptide”
QSVLTQPPSVSGAPGQRVTISCTGSSSNIGAGYDVHWYQQLPGTAPKLLIYGNSNRPNGVPDRFSGSKSGTSASLAITGLQAEDEADYYCQSYDSPTSSSVFGGGTKLTVL

SEQ ID NO: 358 - DNA, 333 bp, Artificial Sequence
  source: /note=“Description of Artificial Sequence Synthetic polynucleotide”
cagagcgtgctgacccagccaccaagcgtgagcggtgcaccaggtcagcgcgtgaccattagctgcaccggcagcagcagcaacattggcgcaggctatgatgtgcattggtatcagcagctgccaggcaccgcaccgaaactgctgatttatggcaacagcaatcgcccaaacggtgtgccggatcgctttagcggcagcaaaagcggcaccagcgccagcctggcgattaccggtctgcaagccgaagacgaagccgattattactgccagtcttacgactctccgacttcttcttctgtgtttggcggcggtaccaagctgaccgtgctg

SEQ ID NO: 359 - protein, 217 aa, Artificial Sequence
  source: /note=“Description of Artificial Sequence Synthetic polypeptide”
QSVLTQPPSVSGAPGQRVTISCTGSSSNIGAGYDVHWYQQLPGTAPKLLIYGNSNRPNGVPDRFSGSKSGTSASLAITGLQAEDEADYYCQSYDSPTSSSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 360 - DNA, 651 bp, Artificial Sequence
  source: /note=“Description of Artificial Sequence Synthetic polynucleotide”
cagagcgtgctgacccagccaccaagcgtgagcggtgcaccaggtcagcgcgtgaccattagctgcaccggcagcagcagcaacattggcgcaggctatgatgtgcattggtatcagcagctgccaggcaccgcaccgaaactgctgatttatggcaacagcaatcgcccaaacggtgtgccggatcgctttagcggcagcaaaagcggcaccagcgccagcctggcgattaccggtctgcaagccgaagacgaagccgattattactgccagtcttacgactctccgacttcttcttctgtgtttggcggcggtaccaagctgaccgtgctgggccagcccaaagccgcccctagcgtgaccctgttccccccaagcagcgaggaactccaggccaacaaggccaccctcgtgtgcctgatcagcgacttctaccctggcgccgtgaccgtggcctggaaggccgatagcagccctgtgaaggccggcgtggaaaccaccacccccagcaagcagagcaacaacaaatacgccgccagcagctacctgagcctgacccccgagcagtggaagtcccacagatcctacagctgccaggtcacacacgagggcagcaccgtggaaaagaccgtggcccccaccgagtgcagc

SEQ ID NO: 361 - protein, 11 aa, Artificial Sequence
  source: /note=“Description of Artificial Sequence Synthetic peptide”
QSYGAFPRFVV

SEQ ID NO: 362 - protein, 8 aa, Artificial Sequence
  source: /note=“Description of Artificial Sequence Synthetic peptide”
YGAFPRFV

SEQ ID NO: 363 - protein, 111 aa, Artificial Sequence
  source: /note=“Description of Artificial Sequence Synthetic polypeptide”
QSVLTQPPSVSGAPGQRVTISCTGSSSNIGAGYDVHWYQQLPGTAPKLLIYGNSNRPNGVPDRFSGSKSGTSASLAITGLQAEDEADYYCQSYGAFPRFVVFGGGTKLTVL

SEQ ID NO: 364 - DNA, 333 bp, Artificial Sequence
  source: /note=“Description of Artificial Sequence Synthetic polynucleotide”
cagagcgtgctgacccagccaccaagcgtgagcggtgcaccaggtcagcgcgtgaccattagctgcaccggcagcagcagcaacattggcgcaggctatgatgtgcattggtatcagcagctgccaggcaccgcaccgaaactgctgatttatggcaacagcaatcgcccaaacggtgtgccggatcgctttagcggcagcaaaagcggcaccagcgccagcctggcgattaccggtctgcaagccgaagacgaagccgattattactgccaatcctatggtgccttccctcgtttcgttgtttttggcggcggtaccaagctgaccgtgctg

SEQ ID NO: 365 - protein, 217 aa, Artificial Sequence
  source: /note=“Description of Artificial Sequence Synthetic polypeptide”
QSVLTQPPSVSGAPGQRVTISCTGSSSNIGAGYDVHWYQQLPGTAPKLLIYGNSNRPNGVPDRFSGSKSGTSASLAITGLQAEDEADYYCQSYGAFPRFVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 366 - DNA, 651 bp, Artificial Sequence
  source: /note=“Description of Artificial Sequence Synthetic polynucleotide”
cagagcgtgctgacccagccaccaagcgtgagcggtgcaccaggtcagcgcgtgaccattagctgcaccggcagcagcagcaacattggcgcaggctatgatgtgcattggtatcagcagctgccaggcaccgcaccgaaactgctgatttatggcaacagcaatcgcccaaacggtgtgccggatcgctttagcggcagcaaaagcggcaccagcgccagcctggcgattaccggtctgcaagccgaagacgaagccgattattactgccaatcctatggtgccttccctcgtttcgttgtttttggcggcggtaccaagctgaccgtgctgggccagcccaaagccgcccctagcgtgaccctgttccccccaagcagcgaggaactccaggccaacaaggccaccctcgtgtgcctgatcagcgacttctaccctggcgccgtgaccgtggcctggaaggccgatagcagccctgtgaaggccggcgtggaaaccaccacccccagcaagcagagcaacaacaaatacgccgccagcagctacctgagcctgacccccgagcagtggaagtcccacagatcctacagctgccaggtcacacacgagggcagcaccgtggaaaagaccgtggcccccaccgagtgcagc

SEQ ID NO: 367 - protein, 10 aa, Artificial Sequence
  source: /note=“Description of Artificial Sequence Synthetic peptide”
GFSFSKYYLN

SEQ ID NO: 368 - protein, 17 aa, Artificial Sequence
  source: /note=“Description of Artificial Sequence Synthetic peptide”
SIHQQAHEKKYVESVKG

SEQ ID NO: 369 - protein, 5 aa, Artificial Sequence
  source: /note=“Description of Artificial Sequence Synthetic peptide”
KYYLN

SEQ ID NO: 370 - protein, 7 aa, Artificial Sequence
  source: /note=“Description of Artificial Sequence Synthetic peptide”
GFSFSKY

SEQ ID NO: 371 - protein, 6 aa, Artificial Sequence
  source: /note=“Description of Artificial Sequence Synthetic peptide”
HQQAHE

SEQ ID NO: 372 - protein, 8 aa, Artificial Sequence
  source: /note=“Description of Artificial Sequence Synthetic peptide”
GFSFSKYY

SEQ ID NO: 373 - protein, 8 aa, Artificial Sequence
  source: /note=“Description of Artificial Sequence Synthetic peptide”
IHQQAHEK

SEQ ID NO: 374 - protein, 123 aa, Artificial Sequence
  source: /note=“Description of Artificial Sequence Synthetic polypeptide”
EVQLVESGGGLVQPGGSLRLSCAASGFSFSKYYLNWVRQAPGKGLEWVASIHQQAHEKKYVESVKGRFTISRDNAKNSLYLQMNSLRAEDTAVYYCARSLRRRSTEHAGFDVWGQGTLVTVSS

SEQ ID NO: 375 - DNA, 369 bp, Artificial Sequence
  source: /note=“Description of Artificial Sequence Synthetic polynucleotide”
gaagtgcagctggtggaaagcggcggtggcctggtgcagccaggtggtagcctgcgcctgagctgcgccgccagcggctttagcttcagcaaatattacttgaactgggttcgccaggccccaggcaaaggcctggaatgggtggccagcattcaccagcaagcacacgagaaaaaatacgtggagtccgtgaaaggccgctttaccattagccgcgataacgccaaaaacagcctgtatctgcaaatgaacagcctgcgggccgaagataccgccgtgtattattgcgcgcgtagcctgcgtcgtcgtagcactgagcacgcaggattcgacgtttggggccagggcaccctggttactgtctcgagc

SEQ ID NO: 376 - protein, 453 aa, Artificial Sequence
  source: /note=“Description of Artificial Sequence Synthetic polypeptide”
EVQLVESGGGLVQPGGSLRLSCAASGFSFSKYYLNWVRQAPGKGLEWVASIHQQAHEKKYVESVKGRFTISRDNAKNSLYLQMNSLRAEDTAVYYCARSLRRRSTEHAGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - DNA, 1359 bp, Artificial Sequence
  source: /note=“Description of Artificial Sequence Synthetic polynucleotide”
gaagtgcagctggtggaaagcggcggtggcctggtgcagccaggtggtagcctgcgcctgagctgcgccgccagcggctttagcttcagcaaatattacttgaactgggttcgccaggccccaggcaaaggcctggaatgggtggccagcattcaccagcaagcacacgagaaaaaatacgtggagtccgtgaaaggccgctttaccattagccgcgataacgccaaaaacagcctgtatctgcaaatgaacagcctgcgggccgaagataccgccgtgtattattgcgcgcgtagcctgcgtcgtcgtagcactgagcacgcaggattcgacgtttggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 378 - protein, 10 aa, Artificial Sequence
  source: /note=“Description of Artificial Sequence Synthetic peptide”
GFTFSRYYIN

SEQ ID NO: 379 - protein, 17 aa, Artificial Sequence
  source: /note=“Description of Artificial Sequence Synthetic peptide”
SIHQHGLETRYVESVKG

SEQ ID NO: 380 - protein, 5 aa, Artificial Sequence
  source: /note=“Description of Artificial Sequence Synthetic peptide”
RYYIN

SEQ ID NO: 381 - protein, 7 aa, Artificial Sequence
  source: /note=“Description of Artificial Sequence Synthetic peptide”
GFTFSRY

SEQ ID NO: 382 - protein, 6 aa, Artificial Sequence
  source: /note=“Description of Artificial Sequence Synthetic peptide”
HQHGLE

SEQ ID NO: 383 - protein, 8 aa, Artificial Sequence
  source: /note=“Description of Artificial Sequence Synthetic peptide”
GFTFSRYY

SEQ ID NO: 384 - protein, 8 aa, Artificial Sequence
  source: /note=“Description of Artificial Sequence Synthetic peptide”
IHQHGLET

SEQ ID NO: 385 - protein, 123 aa, Artificial Sequence
  source: /note=“Description of Artificial Sequence Synthetic polypeptide”
EVQLVESGGGLVQPGGSLRLSCAASGFTFSRYYINWVRQAPGKGLEWVASIHQHGLETRYVESVKGRFTISRDNAKNSLYLQMNSLRAEDTAVYYCARSLRRRSTEHAGFDVWGQGTLVTVSS

SEQ ID NO: 386 - DNA, 369 bp, Artificial Sequence
  source: /note=“Description of Artificial Sequence Synthetic polynucleotide”
gaagtgcagctggtggaaagcggcggtggcctggtgcagccaggtggtagcctgcgcctgagctgcgccgccagcgggtttactttttccagatattacattaattgggttcgccaggccccaggcaaaggcctggaatgggtggcgagcatccaccagcacggcctggagaccagatatgtggaatctgtcaaagggcgctttaccattagccgcgataacgccaaaaacagcctgtatctgcaaatgaacagcctgcgggccgaagataccgccgtgtattattgcgcgcgtagcctgcgtcgtcgtagcactgagcacgcaggattcgacgtttggggccagggcaccctggttactgtctcgagc

SEQ ID NO: 387 - protein, 453 aa, Artificial Sequence
  source: /note=“Description of Artificial Sequence Synthetic polypeptide”
EVQLVESGGGLVQPGGSLRLSCAASGFTFSRYYINWVRQAPGKGLEWVASIHQHGLETRYVESVKGRFTISRDNAKNSLYLQMNSLRAEDTAVYYCARSLRRRSTEHAGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 388 - DNA, 1359 bp, Artificial Sequence
  source: /note=“Description of Artificial Sequence Synthetic polynucleotide”
gaagtgcagctggtggaaagcggcggtggcctggtgcagccaggtggtagcctgcgcctgagctgcgccgccagcgggtttactttttccagatattacattaattgggttcgccaggccccaggcaaaggcctggaatgggtggcgagcatccaccagcacggcctggagaccagatatgtggaatctgtcaaagggcgctttaccattagccgcgataacgccaaaaacagcctgtatctgcaaatgaacagcctgcgggccgaagataccgccgtgtattattgcgcgcgtagcctgcgtcgtcgtagcactgagcacgcaggattcgacgtttggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 389 - protein, 17 aa, Artificial Sequence
  source: /note=“Description of Artificial Sequence Synthetic peptide”
SISSHGYYTRYAESVKG

SEQ ID NO: 390 - protein, 6 aa, Artificial Sequence
  source: /note=“Description of Artificial Sequence Synthetic peptide”
SSHGYY

SEQ ID NO: 391 - protein, 8 aa, Artificial Sequence
  source: /note=“Description of Artificial Sequence Synthetic peptide”
ISSHGYYT

SEQ ID NO: 392 - protein, 122 aa, Artificial Sequence
  source: /note=“Description of Artificial Sequence Synthetic polypeptide”
EVQLLESGGGLVQPGGSLRLSCAASGFTFSSYAISWVRQAPGKGLEWVSSISSHGYYTRYAESVKGRFTISRDNSKNTLYLQMNSLRAEDTAVYYCARPYLGDRRSYGFDHWGQGTLVTVSS

SEQ ID NO: 393 - DNA, 366 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcgggtttacattttccagctatgctatcagctgggtgcgccaagcaccaggcaaaggcctggaatgggtgagcagcattagctcacatggatattacacccggtatgccgagtccgtgaaaggtcgctttaccattagtcgcgataacagcaaaaacaccctgtatctgcaaatgaacagcctgcgggcagaagataccgcagtttattattgcgcgcgaccttatctgggtgaccgtcgtagctatggtttcgaccactggggccagggcaccctggttactgtctcgagc

SEQ ID NO: 394 - protein, 452 aa, Artificial Sequence
  source: /note=“Description of Artificial Sequence Synthetic polypeptide”
EVQLLESGGGLVQPGGSLRLSCAASGFTFSSYAISWVRQAPGKGLEWVSSISSHGYYTRYAESVKGRFTISRDNSKNTLYLQMNSLRAEDTAVYYCARPYLGDRRSYGFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 395 - DNA, 1356 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcgggtttacattttccagctatgctatcagctgggtgcgccaagcaccaggcaaaggcctggaatgggtgagcagcattagctcacatggatattacacccggtatgccgagtccgtgaaaggtcgctttaccattagtcgcgataacagcaaaaacaccctgtatctgcaaatgaacagcctgcgggcagaagataccgcagtttattattgcgcgcgaccttatctgggtgaccgtcgtagctatggtttcgaccactggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 396 - protein, 10 aa, Artificial Sequence
  source: /note=“Description of Artificial Sequence Synthetic peptide”
GFTFASYAIT

SEQ ID NO: 397 - protein, 17 aa, Artificial Sequence
  source: /note=“Description of Artificial Sequence Synthetic peptide”
TISGSGVYTYYAESVKG

SEQ ID NO: 398 - protein, 5 aa, Artificial Sequence
  source: /note=“Description of Artificial Sequence Synthetic peptide”
SYAIT

SEQ ID NO: 399 - protein, 7 aa, Artificial Sequence
  source: /note=“Description of Artificial Sequence Synthetic peptide”
GFTFASY

SEQ ID NO: 400 - protein, 6 aa, Artificial Sequence
  source: /note=“Description of Artificial Sequence Synthetic peptide”
SGSGVY

SEQ ID NO: 401 - protein, 8 aa, Artificial Sequence
  source: /note=“Description of Artificial Sequence Synthetic peptide”
GFTFASYA

SEQ ID NO: 402 - protein, 8 aa, Artificial Sequence
  source: /note=“Description of Artificial Sequence Synthetic peptide”
ISGSGVYT

SEQ ID NO: 403 - protein, 122 aa, Artificial Sequence
  source: /note=“Description of Artificial Sequence Synthetic polypeptide”
EVQLLESGGGLVQPGGSLRLSCAASGFTFASYAITWVRQAPGKGLEWVSTISGSGVYTYYAESVKGRFTISRDNSKNTLYLQMNSLRAEDTAVYYCARPYLGDRRSYGFDHWGQGTLVTVSS

SEQ ID NO: 404 - DNA, 366 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcgggttcacattcgcatcctatgcaattacttgggtgcgccaagcaccaggcaaaggcctggaatgggtgagcaccatttccgggtccggtgtgtacacctattacgccgagtccgtcaaaggccgctttaccattagtcgcgataacagcaaaaacaccctgtatctgcaaatgaacagcctgcgggcagaagataccgcagtttattattgcgcgcgaccttatctgggtgaccgtcgtagctatggtttcgaccactggggccagggcaccctggttactgtctcgagc

SEQ ID NO: 405 - protein, 452 aa, Artificial Sequence
  source: /note=“Description of Artificial Sequence Synthetic polypeptide”
EVQLLESGGGLVQPGGSLRLSCAASGFTFASYAITWVRQAPGKGLEWVSTISGSGVYTYYAESVKGRFTISRDNSKNTLYLQMNSLRAEDTAVYYCARPYLGDRRSYGFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 406 - DNA, 1356 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcgggttcacattcgcatcctatgcaattacttgggtgcgccaagcaccaggcaaaggcctggaatgggtgagcaccatttccgggtccggtgtgtacacctattacgccgagtccgtcaaaggccgctttaccattagtcgcgataacagcaaaaacaccctgtatctgcaaatgaacagcctgcgggcagaagataccgcagtttattattgcgcgcgaccttatctgggtgaccgtcgtagctatggtttcgaccactggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 407 - protein, 10 aa, Artificial Sequence
  source: /note=“Description of Artificial Sequence Synthetic peptide”
GFTFGTYAMT

SEQ ID NO: 408 - protein, 17 aa, Artificial Sequence
  source: /note=“Description of Artificial Sequence Synthetic peptide”
SISASGYYANYAGSVKG

SEQ ID NO: 409 - protein, 5 aa, Artificial Sequence
  source: /note=“Description of Artificial Sequence Synthetic peptide”
TYAMT

SEQ ID NO: 410 - protein, 7 aa, Artificial Sequence
  source: /note=“Description of Artificial Sequence Synthetic peptide”
GFTFGTY

SEQ ID NO: 411 - protein, 6 aa, Artificial Sequence
  source: /note=“Description of Artificial Sequence Synthetic peptide”
SASGYY

SEQ ID NO: 412 - protein, 8 aa, Artificial Sequence
  source: /note=“Description of Artificial Sequence Synthetic peptide”
GFTFGTYA

SEQ ID NO: 413 - protein, 5 aa, Artificial Sequence
  source: /note=“Description of Artificial Sequence Synthetic peptide”
ISASG

SEQ ID NO: 414 - protein, 122 aa, Artificial Sequence
  source: /note=“Description of Artificial Sequence Synthetic polypeptide”
EVQLLESGGGLVQPGGSLRLSCAASGFTFGTYAMTWVRQAPGKGLEWVSSISASGYYANYAGSVKGRFTISRDNSKNTLYLQMNSLRAEDTAVYYCARPYLGDRRSYGFDHWGQGTLVTVSS

SEQ ID NO: 415 - DNA, 366 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcgggtttacattcggcacctatgcaatgacttgggtgcgccaagcaccaggcaaaggcctggaatgggtgagtagcattagcgcatccggatattacgctaactacgcaggcagcgtcaaaggccgctttaccattagtcgcgataacagcaaaaacaccctgtatctgcaaatgaacagcctgcgggcagaagataccgcagtttattattgcgcgcgaccttatctgggtgaccgtcgtagctatggtttcgaccactggggccagggcaccctggttactgtctcgagc

SEQ ID NO: 416 - protein, 452 aa, Artificial Sequence
  source: /note=“Description of Artificial Sequence Synthetic polypeptide”
EVQLLESGGGLVQPGGSLRLSCAASGFTFGTYAMTWVRQAPGKGLEWVSSISASGYYANYAGSVKGRFTISRDNSKNTLYLQMNSLRAEDTAVYYCARPYLGDRRSYGFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 417 - DNA, 1356 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcgggtttacattcggcacctatgcaatgacttgggtgcgccaagcaccaggcaaaggcctggaatgggtgagtagcattagcgcatccggatattacgctaactacgcaggcagcgtcaaaggccgctttaccattagtcgcgataacagcaaaaacaccctgtatctgcaaatgaacagcctgcgggcagaagataccgcagtttattattgcgcgcgaccttatctgggtgaccgtcgtagctatggtttcgaccactggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 418 - protein, 10 aa, Artificial Sequence
  source: /note=“Description of Artificial Sequence Synthetic peptide”
GFTFSDYAIS

SEQ ID NO: 419 - protein, 17 aa, Artificial Sequence
  source: /note=“Description of Artificial Sequence Synthetic peptide”
SISGGGYHTQYAGSVKG

SEQ ID NO: 420 - protein, 5 aa, Artificial Sequence
  source: /note=“Description of Artificial Sequence Synthetic peptide”
DYAIS

SEQ ID NO: 421 - protein, 7 aa, Artificial Sequence
  source: /note=“Description of Artificial Sequence Synthetic peptide”
GFTFSDY

SEQ ID NO: 422 - protein, 6 aa, Artificial Sequence
  source: /note=“Description of Artificial Sequence Synthetic peptide”
SGGGYH

SEQ ID NO: 423 - protein, 8 aa, Artificial Sequence
  source: /note=“Description of Artificial Sequence Synthetic peptide”
GFTFSDYA

SEQ ID NO: 424 - protein, 8 aa, Artificial Sequence
  source: /note=“Description of Artificial Sequence Synthetic peptide”
ISGGGYHT

SEQ ID NO: 425 - protein, 122 aa, Artificial Sequence
  source: /note=“Description of Artificial Sequence Synthetic polypeptide”
EVQLLESGGGLVQPGGSLRLSCAASGFTFSDYAISWVRQAPGKGLEWVSSISGGGYHTQYAGSVKGRFTISRDNSKNTLYLQMNSLRAEDTAVYYCARPYLGDRRSYGFDHWGQGTLVTVSS

SEQ ID NO: 426 - DNA, 366 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcggctttaccttttccgactatgcaatcagctgggtgcgccaagcaccaggcaaaggcctggaatgggtgagcagcatttccgggggggggtatcatacacaatatgcaggatccgtgaaaggccgctttaccattagtcgcgataacagcaaaaacaccctgtatctgcaaatgaacagcctgcgggcagaagataccgcagtttattattgcgcgcgaccttatctgggtgaccgtcgtagctatggtttcgaccactggggccagggcaccctggttactgtctcgagc

SEQ ID NO: 427 - protein, 452 aa, Artificial Sequence
  source: /note=“Description of Artificial Sequence Synthetic polypeptide”
EVQLLESGGGLVQPGGSLRLSCAASGFTFSDYAISWVRQAPGKGLEWVSSISGGGYHTQYAGSVKGRFTISRDNSKNTLYLQMNSLRAEDTAVYYCARPYLGDRRSYGFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 428 - DNA, 1356 bp, Artificial Sequence
  source: /note=“Description of Artificial Sequence Synthetic polynucleotide”
gaagtgcagctgctggaaagcggtggcggtctggtgcagccaggtggtagcctgcgcctgagctgtgccgcaagcggctttaccttttccgactatgcaatcagctgggtgcgccaagcaccaggcaaaggcctggaatgggtgagcagcatttccgggggggggtatcatacacaatatgcaggatccgtgaaaggccgctttaccattagtcgcgataacagcaaaaacaccctgtatctgcaaatgaacagcctgcgggcagaagataccgcagtttattattgcgcgcgaccttatctgggtgaccgtcgtagctatggtttcgaccactggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggcgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 429 - protein, 17 aa, Artificial Sequence
  source: /note=“Description of Artificial Sequence Synthetic peptide”
  VARIANT (1)..(1): /replace=“V”
  VARIANT (3)..(3): /replace=“E”
  VARIANT (5)..(5): /replace=“K”
  VARIANT (7)..(7): /replace=“N”
  VARIANT (9)..(9): /replace=“T”
  VARIANT (10)..(10): /replace=“F”
  SITE (1)..(17): /note=“Variant residues given in the sequence have no preference with respect to those in the annotations for variant positions”
AISSDGSYIYYADSVKG

SEQ ID NO: 430 - protein, 9 aa, Artificial Sequence
  source: /note=“Description of Artificial Sequence Synthetic peptide”
  VARIANT (1)..(1): /replace=“Q”
  VARIANT (3)..(3): /replace=“E” or “T” or “I”
  VARIANT (4)..(4): /replace=“W”
  VARIANT (5)..(5): /replace=“V” or “R” or “A” or “T” or “M”
  VARIANT (6)..(6): /replace=“V” or “R” or “A”
  SITE (1)..(9): /note=“Variant residues given in the sequence have no preference with respect to those in the annotations for variant positions”
MQSYEKPRT

SEQ ID NO: 431 - protein, 6 aa, Artificial Sequence
  source: /note=“Description of Artificial Sequence Synthetic peptide”
  VARIANT (1)..(1): /replace=“E”
  VARIANT (3)..(3): /replace=“K”
  VARIANT (5)..(5): /replace=“N”
  SITE (1)..(6): /note=“Variant residues given in the sequence have no preference with respect to those in the annotations for variant positions”
SSDGSY

SEQ ID NO: 432 - protein, 6 aa, Artificial Sequence
  source: /note=“Description of Artificial Sequence Synthetic peptide”
  VARIANT (1)..(1): /replace=“E” or “T” or “I”
  VARIANT (2)..(2): /replace=“W”
  VARIANT (3)..(3): /replace=“V” or “R” or “A” or “T” or “M”
  VARIANT (4)..(4): /replace=“V” or “R” or “A”
  SITE (1)..(6): /note=“Variant residues given in the sequence have no preference with respect to those in the annotations for variant positions”
SYEKPR

SEQ ID NO: 433 - protein, 8 aa, Artificial Sequence
  source: /note=“Description of Artificial Sequence Synthetic peptide”
  VARIANT (2)..(2): /replace=“E”
  VARIANT (4)..(4): /replace=“K”
  VARIANT (6)..(6): /replace=“N”
  VARIANT (8)..(8): /replace=“T”
  SITE (1)..(8): /note=“Variant residues given in the sequence have no preference with respect to those in the annotations for variant positions”
ISSDGSYI

SEQ ID NO: 434 - protein, 9 aa, Artificial Sequence
  source: /note=“Description of Artificial Sequence Synthetic peptide”
  VARIANT (3)..(3): /replace=“E” or “T” or “I”
  VARIANT (5)..(5): /replace=“R” or “A” or “T” or “M”
  VARIANT (6)..(6): /replace=“V” or “R” or “A”
  SITE (1)..(9): /note=“Variant residues given in the sequence have no preference with respect to those in the annotations for variant positions”
QQSWVKPRT

SEQ ID NO: 435 - protein, 6 aa, Artificial Sequence
  source: /note=“Description of Artificial Sequence Synthetic peptide”
  VARIANT (1)..(1): /replace=“E” or “T” or “I”
  VARIANT (3)..(3): /replace=“R” or “A” or “T” or “M”
  VARIANT (4)..(4): /replace=“V” or “R” or “A”
  SITE (1)..(6): /note=“Variant residues given in the sequence have no preference with respect to those in the annotations for variant positions”
SWVKPR

SEQ ID NO: 436 - protein, 10 aa, Artificial Sequence
  source: /note=“Description of Artificial Sequence Synthetic peptide”
  VARIANT (5)..(5): /replace=“S” or “Q”
  SITE (1)..(10): /note=“Variant residues given in the sequence have no preference with respect to those in the annotations for variant positions”
GFTFNTHYIH

SEQ ID NO: 437 - protein, 17 aa, Artificial Sequence
  source: /note=“Description of Artificial Sequence Synthetic peptide”
  VARIANT (3)..(3): /replace=“G”
  VARIANT (4)..(4): /replace=“G”
  VARIANT (5)..(5): /replace=“Q”
  VARIANT (7)..(7): /replace=“Q” or “G”
  VARIANT (8)..(8): /replace=“N” or “M”
  VARIANT (10)..(10): /replace=“L”
  SITE (1)..(17): /note=“Variant residues given in the sequence have no preference with respect to those in the annotations for variant positions”
SISSSGSSTYYADSVKG

SEQ ID NO: 438 - protein, 7 aa, Artificial Sequence
  source: /note=“Description of Artificial Sequence Synthetic peptide”
  VARIANT (5)..(5): /replace=“S” or “Q”
  SITE (1)..(7): /note=“Variant residues given in the sequence have no preference with respect to those in the annotations for variant positions”
GFTFNTH

SEQ ID NO: 439 - protein, 6 aa, Artificial Sequence
  source: /note=“Description of Artificial Sequence Synthetic peptide”
  VARIANT (1)..(1): /replace=“G”
  VARIANT (2)..(2): /replace=“G”
  VARIANT (3)..(3): /replace=“Q”
  VARIANT (5)..(5): /replace=“Q” or “G”
  VARIANT (6)..(6): /replace=“N” or “M”
  SITE (1)..(6): /note=“Variant residues given in the sequence have no preference with respect to those in the annotations for variant positions”
SSSGSS

SEQ ID NO: 440 - protein, 8 aa, Artificial Sequence
  source: /note=“Description of Artificial Sequence Synthetic peptide”
  VARIANT (5)..(5): /replace=“S” or “Q”
  SITE (1)..(8): /note=“Variant residues given in the sequence have no preference with respect to those in the annotations for variant positions”
GFTFNTHY

SEQ ID NO: 441 - protein, 8 aa, Artificial Sequence
  source: /note=“Description of Artificial Sequence Synthetic peptide”
  VARIANT (2)..(2): /replace=“G”
  VARIANT (3)..(3): /replace=“G”
  VARIANT (4)..(4): /replace=“Q”
  VARIANT (6)..(6): /replace=“Q” or “G”
  VARIANT (7)..(7): /replace=“N” or “M”
  SITE (1)..(8): /note=“Variant residues given in the sequence have no preference with respect to those in the annotations for variant positions”
ISSSGSST

SEQ ID NO: 442 - protein, 17 aa, Artificial Sequence
  source: /note=“Description of Artificial Sequence Synthetic peptide”
  VARIANT (4)..(4): /replace=“G”
  VARIANT (7)..(7): /replace=“Q”
  VARIANT (8)..(8): /replace=“N”
  SITE (1)..(17): /note=“Variant residues given in the sequence have no preference with respect to those in the annotations for variant positions”
SISSSGSSTYYADSVKG

SEQ ID NO: 443 - protein, 6 aa, Artificial Sequence
  source: /note=“Description of Artificial Sequence Synthetic peptide”
  VARIANT (2)..(2): /replace=“G”
  VARIANT (5)..(5): /replace=“Q”
  VARIANT (6)..(6): /replace=“N”
  SITE (1)..(6): /note=“Variant residues given in the sequence have no preference with respect to those in the annotations for variant positions”
SSSGSS

SEQ ID NO: 444 - protein, 8 aa, Artificial Sequence
  source: /note=“Description of Artificial Sequence Synthetic peptide”
  VARIANT (3)..(3): /replace=“G”
  VARIANT (6)..(6): /replace=“Q”
  VARIANT (7)..(7): /replace=“N”
  SITE (1)..(8): /note=“Variant residues given in the sequence have no preference with respect to those in the annotations for variant positions”
ISSSGSST

SEQ ID NO: 445 - protein, 10 aa, Artificial Sequence
  source: /note=“Description of Artificial Sequence Synthetic peptide”
  VARIANT (3)..(3): /replace=“T”
  VARIANT (6)..(6): /replace=“K” or “R”
  VARIANT (8)..(8): /replace=“Y”
  VARIANT (9)..(9): /replace=“L”
  VARIANT (10)..(10): /replace=“N”
  SITE (1)..(10): /note=“Variant residues given in the sequence have no preference with respect to those in the annotations for variant positions”
GFSFSSYWIS

SEQ ID NO: 446 - protein, 17 aa, Artificial Sequence
  source: /note=“Description of Artificial Sequence Synthetic peptide”
  VARIANT (1)..(1): /replace=“N”
  VARIANT (3)..(3): /replace=“H”
  VARIANT (5)..(5): /replace=“Q” or “H”
  VARIANT (6)..(6): /replace=“A”
  VARIANT (7)..(7): /replace=“H” or “L”
  VARIANT (9)..(9): /replace=“K”
  VARIANT (10)..(10): /replace=“K” or “R”
  SITE (1)..(17): /note=“Variant residues given in the sequence have no preference with respect to those in the annotations for variant positions”
SIKQSGSETYYVESVKG

SEQ ID NO: 447 - protein, 5 aa, Artificial Sequence
  source: /note=“Description of Artificial Sequence Synthetic peptide”
  VARIANT (1)..(1): /replace=“K” or “R”
  VARIANT (3)..(3): /replace=“Y”
  VARIANT (4)..(4): /replace=“L”
  VARIANT (5)..(5): /replace=“N”
  SITE (1)..(5): /note=“Variant residues given in the sequence have no preference with respect to those in the annotations for variant positions”
SYWIS

SEQ ID NO: 448 - protein, 7 aa, Artificial Sequence
  source: /note=“Description of Artificial Sequence Synthetic peptide”
  VARIANT (3)..(3): /replace=“T”
  VARIANT (6)..(6): /replace=“K” or “R”
  SITE (1)..(7): /note=“Variant residues given in the sequence have no preference with respect to those in the annotations for variant positions”
GFSFSSY

SEQ ID NO: 449 - protein, 6 aa, Artificial Sequence
  source: /note=“Description of Artificial Sequence Synthetic peptide”
  VARIANT (1)..(1): /replace=“H”
  VARIANT (3)..(3): /replace=“Q” or “H”
  VARIANT (4)..(4): /replace=“A”
  VARIANT (5)..(5): /replace=“H” or “L”
  SITE (1)..(6): /note=“Variant residues given in the sequence have no preference with respect to those in the annotations for variant positions”
KQSGSE

SEQ ID NO: 450 - protein, 8 aa, Artificial Sequence
  source: /note=“Description of Artificial Sequence Synthetic peptide”
  VARIANT (3)..(3): /replace=“T”
  VARIANT (6)..(6): /replace=“K” or “R”
  VARIANT (8)..(8): /replace=“Y”
  SITE (1)..(8): /note=“Variant residues given in the sequence have no preference with respect to those in the annotations for variant positions”
GFSFSSYW

SEQ ID NO: 451 - protein, 8 aa, Artificial Sequence
  source: /note=“Description of Artificial Sequence Synthetic peptide”
  VARIANT (2)..(2): /replace=“H”
  VARIANT (4)..(4): /replace=“Q” or “H”
  VARIANT (5)..(5): /replace=“A”
  VARIANT (6)..(6): /replace=“H” or “L”
  VARIANT (8)..(8): /replace=“K”
  SITE (1)..(8): /note=“Variant residues given in the sequence have no preference with respect to those in the annotations for variant positions”
IKQSGSET

SEQ ID NO: 452 - protein, 10 aa, Artificial Sequence
  source: /note=“Description of Artificial Sequence Synthetic peptide”
  VARIANT (6)..(6): /replace=“R”
  VARIANT (8)..(8): /replace=“Y”
  VARIANT (10)..(10): /replace=“N”
  SITE (1)..(10): /note=“Variant residues given in the sequence have no preference with respect to those in the annotations for variant positions”
GFTFSSYWIS

SEQ ID NO: 453 - protein, 17 aa, Artificial Sequence
  source: /note=“Description of Artificial Sequence Synthetic peptide”
  VARIANT (5)..(5): /replace=“H”
  VARIANT (6)..(6): /replace=“A”
  VARIANT (7)..(7): /replace=“L”
  VARIANT (9)..(9): /replace=“K”
  VARIANT (10)..(10): /replace=“R”
  SITE (1)..(17): /note=“Variant residues given in the sequence have no preference with respect to those in the annotations for variant positions”
SIHQQGHETKYVESVKG

SEQ ID NO: 454 - protein, 5 aa, Artificial Sequence
  source: /note=“Description of Artificial Sequence Synthetic peptide”
  VARIANT (1)..(1): /replace=“R”
  VARIANT (3)..(3): /replace=“Y”
  VARIANT (5)..(5): /replace=“N”
  SITE (1)..(5): /note=“Variant residues given in the sequence have no preference with respect to those in the annotations for variant positions”
SYWIS

SEQ ID NO: 455 - protein, 7 aa, Artificial Sequence
  source: /note=“Description of Artificial Sequence Synthetic peptide”
  VARIANT (6)..(6): /replace=“R”
  SITE (1)..(7): /note=“Variant residues given in the sequence have no preference with respect to those in the annotations for variant positions”
GFTFSSY

SEQ ID NO: 456 - protein, 6 aa, Artificial Sequence
  source: /note=“Description of Artificial Sequence Synthetic peptide”
  VARIANT (3)..(3): /replace=“H”
  VARIANT (4)..(4): /replace=“A”
  VARIANT (5)..(5): /replace=“L”
  SITE (1)..(6): /note=“Variant residues given in the sequence have no preference with respect to those in the annotations for variant positions”
HQQGHE

SEQ ID NO: 457 - protein, 8 aa, Artificial Sequence
  source: /note=“Description of Artificial Sequence Synthetic peptide”
  VARIANT (6)..(6): /replace=“R”
  VARIANT (8)..(8): /replace=“Y”
  SITE (1)..(8): /note=“Variant residues given in the sequence have no preference with respect to those in the annotations for variant positions”
GFTFSSYW

SEQ ID NO: 458 - protein, 8 aa, Artificial Sequence
  source: /note=“Description of Artificial Sequence Synthetic peptide”
  VARIANT (4)..(4): /replace=“H”
  VARIANT (5)..(5): /replace=“A”
  VARIANT (6)..(6): /replace=“L”
  VARIANT (8)..(8): /replace=“K”
  SITE (1)..(8): /note=“Variant residues given in the sequence have no preference with respect to those in the annotations for variant positions”
IHQQGHET

SEQ ID NO: 459 - protein, 11 aa, Artificial Sequence
  source: /note=“Description of Artificial Sequence Synthetic peptide”
  VARIANT (2)..(2): /replace=“S”
  VARIANT (3)..(3): /replace=“L”
  VARIANT (4)..(4): /replace=“P”
  VARIANT (6)..(6): /replace=“L”
  SITE (1)..(11): /note=“Variant residues given in the sequence have no preference with respect to those in the annotations for variant positions”
GAVAGQLGFDH

SEQ ID NO: 460 - protein, 7 aa, Artificial Sequence
  source: /note=“Description of Artificial Sequence Synthetic peptide”
  VARIANT (7)..(7): /replace=“N”
  SITE (1)..(7): /note=“Variant residues given in the sequence have no preference with respect to those in the annotations for variant positions”
GNSNRPS

SEQ ID NO: 461 - protein, 11 aa, Artificial Sequence
  source: /note=“Description of Artificial Sequence Synthetic peptide”
  VARIANT (4)..(4): /replace=“D” or “G”
  VARIANT (5)..(5): /replace=“S” or “A”
  VARIANT (6)..(6): /replace=“P” or “F”
  VARIANT (7)..(7): /replace=“T” or “P”
  VARIANT (8)..(8): /replace=“S” or “R”
  VARIANT (9)..(9): /replace=“S” or “F”
  VARIANT (10)..(10): /replace=“S” or “V”
  SITE (1)..(11): /note=“Variant residues given in the sequence have no preference with respect to those in the annotations for variant positions”
QSYYTSSHGPV

SEQ ID NO: 462 - protein, 8 aa, Artificial Sequence
  source: /note=“Description of Artificial Sequence Synthetic peptide”
  VARIANT (2)..(2): /replace=“D” or “G”
  VARIANT (3)..(3): /replace=“S” or “A”
  VARIANT (4)..(4): /replace=“P” or “F”
  VARIANT (5)..(5): /replace=“T” or “P”
  VARIANT (6)..(6): /replace=“S” or “R”
  VARIANT (7)..(7): /replace=“S” or “F”
  VARIANT (8)..(8): /replace=“S” or “V”
  SITE (1)..(8): /note=“Variant residues given in the sequence have no preference with respect to those in the annotations for variant positions”
YYTSSHGP

SEQ ID NO: 463 - protein, 13 aa, Artificial Sequence
  source: /note=“Description of Artificial Sequence Synthetic peptide”
  VARIANT (4)..(4): /replace=“S”
  VARIANT (5)..(5): /replace=“L”
  VARIANT (6)..(6): /replace=“P”
  VARIANT (8)..(8): /replace=“L”
  SITE (1)..(13): /note=“Variant residues given in the sequence have no preference with respect to those in the annotations for variant positions”
ARGAVAGQLGFDH

SEQ ID NO: 464 - protein, 10 aa, Artificial Sequence
  source: /note=“Description of Artificial Sequence Synthetic peptide”
  VARIANT (5)..(5): /replace=“G”
  VARIANT (6)..(6): /replace=“T”
  VARIANT (9)..(9): /replace=“M”
  VARIANT (10)..(10): /replace=“T”
  SITE (1)..(10): /note=“Variant residues given in the sequence have no preference with respect to those in the annotations for variant positions”
GFTFSSYAIS

SEQ ID NO: 465 - protein, 17 aa, Artificial Sequence
  source: /note=“Description of Artificial Sequence Synthetic peptide”
  VARIANT (1)..(1): /replace=“S”
  VARIANT (4)..(4): /replace=“S” or “G”
  VARIANT (5)..(5): /replace=“H”
  VARIANT (7)..(7): /replace=“Y”
  VARIANT (8)..(8): /replace=“Y”
  VARIANT (9)..(9): /replace=“A”
  VARIANT (10)..(10): /replace=“R” or “N”
  VARIANT (13)..(13): /replace=“G”
  SITE (1)..(17): /note=“Variant residues given in the sequence have no preference with respect to those in the annotations for variant positions”
AISASGGSTYYAESVKG

SEQ ID NO: 466 - protein, 5 aa, Artificial Sequence
  source: /note=“Description of Artificial Sequence Synthetic peptide”
  VARIANT (1)..(1): /replace=“T”
  VARIANT (4)..(4): /replace=“M”
  VARIANT (5)..(5): /replace=“T”
  SITE (1)..(5): /note=“Variant residues given in the sequence have no preference with respect to those in the annotations for variant positions”
SYAIS

SEQ ID NO: 467 - protein, 7 aa, Artificial Sequence
  source: /note=“Description of Artificial Sequence Synthetic peptide”
  VARIANT (5)..(5): /replace=“G”
  VARIANT (6)..(6): /replace=“T”
  SITE (1)..(7): /note=“Variant residues given in the sequence have no preference with respect to those in the annotations for variant positions”
GFTFSSY

SEQ ID NO: 468 - protein, 6 aa, Artificial Sequence
  source: /note=“Description of Artificial Sequence Synthetic peptide”
  VARIANT (2)..(2): /replace=“S” or “G”
  VARIANT (3)..(3): /replace=“H”
  VARIANT (5)..(5): /replace=“Y”
  VARIANT (6)..(6): /replace=“Y”
  SITE (1)..(6): /note=“Variant residues given in the sequence have no preference with respect to those in the annotations for variant positions”
SASGGS

SEQ ID NO: 469 - protein, 8 aa, Artificial Sequence
  source: /note=“Description of Artificial Sequence Synthetic peptide”
  VARIANT (5)..(5): /replace=“G”
  VARIANT (6)..(6): /replace=“T”
  SITE (1)..(8): /note=“Variant residues given in the sequence have no preference with respect to those in the annotations for variant positions”
GFTFSSYA

SEQ ID NO: 470 - protein, 8 aa, Artificial Sequence
  source: /note=“Description of Artificial Sequence Synthetic peptide”
  VARIANT (3)..(3): /replace=“G”
  VARIANT (4)..(4): /replace=“H”
  VARIANT (6)..(6): /replace=“Y”
  VARIANT (7)..(7): /replace=“Y”
  SITE (1)..(8): /note=“Variant residues given in the sequence have no preference with respect to those in the annotations for variant positions”
ISSSGGST

SEQ ID NO: 471 - protein, 17 aa, Artificial Sequence
  source: /note=“Description of Artificial Sequence Synthetic peptide”
  VARIANT (4)..(4): /replace=“S”
  VARIANT (5)..(5): /replace=“H”
  VARIANT (9)..(9): /replace=“A”
  VARIANT (10)..(10): /replace=“N”
  VARIANT (13)..(13): /replace=“G”
  SITE (1)..(17): /note=“Variant residues given in the sequence have no preference with respect to those in the annotations for variant positions”
SISASGYYTRYAESVKG

SEQ ID NO: 472 - protein, 6 aa, Artificial Sequence
  source: /note=“Description of Artificial Sequence Synthetic peptide”
  VARIANT (2)..(2): /replace=“S”
  VARIANT (3)..(3): /replace=“H”
  SITE (1)..(6): /note=“Variant residues given in the sequence have no preference with respect to those in the annotations for variant positions”
SASGYY

SEQ ID NO: 473 - protein, 5 aa, Artificial Sequence
  source: /note=“Description of Artificial Sequence Synthetic peptide”
  VARIANT (3)..(3): /replace=“S” or “G”
  VARIANT (4)..(4): /replace=“H”
  SITE (1)..(5): /note=“Variant residues given in the sequence have no preference with respect to those in the annotations for variant positions”
ISASG